SEQUENCE LISTING (6 sequences)

SEQ ID NO: 1 - protein, 1085 aa, Plasmodium falciparum
MKLTKGCAYKYIIFTVLILANILYDNKKRCMIKKNLRISSCGIISRLLKSNSNYNSFNKNYNFTSAISELQFSNFWNLDILQKDIFSNIHNNKNKPQSYIIHKRLMSEKGDNNNNNHQNNNGNDNKKRLGSVVNNEENTCSDKRMKPFEEGHGITQVDKMNNNSDHLQQNGVMNLNSNNVENNNNNNSVVVKKNEPKIHYRKDYKPSGFIINNVTLNINIHDNETIVRSVLDMDISKHNVGEDLVFDGVGLKINEISINNKKLVEGEEYTYDNEFLTIFSKFVPKSKFAFSSEVIIHPETNYALTGLYKSKNIIVSQCEATGFRRITFFIDRPDMMAKYDVTVTADKEKYPVLLSNGDKVNEFEIPGGRHGARFNDPHLKPCYLFAVVAGDLKHLSATYITKYTKKKVELYVFSEEKYVSKLQWALECLKKSMAFDEDYFGLEYDLSRLNLVAVSDFNVGAMENKGLNIFNANSLLASKKNSIDFSYARILTVVGHEYFHNYTGNRVTLRDWFQLTLKEGLTVHRENLFSEEMTKTVTTRLSHVDLLRSVQFLEDSSPLSHPIRPESYVSMENFYTTTVYDKGSEVMRMYLTILGEEYYKKGFDIYIKKNDGNTATCEDFNYAMEQAYKMKKADNSANLNQYLLWFSQSGTPHVSFKYNYDAEKKQYSIHVNQYTKPDENQKEKKPLFIPISVGLINPENGKEMISQTTLELTKESDTFVFNNIAVKPIPSLFRGFSAPVYIEDNLTDEERILLLKYDSDAFVRYNSCTNIYMKQILMNYNEFLKAKNEKLESFNLTPVNAQFIDAIKYLLEDPHADAGFKSYIVSLPQDRYIINFVSNLDTDVLADTKEYIYKQIGDKLNDVYYKMFKSLEAKADDLTYFNDESHVDFDQMNMRTLRNTLLSLLSKAQYPNILNEIIEHSKSPYPSNWLTSLSVSAYFDKYFELYDKTYKLSKDDELLLQEWLKTVSRSDRKDIYEILKKLENEVLKDSKNPNDIRAVYLPFTNNLRRFHDISGKGYKLIAEVITKTDKFNPMVATQLCEPFKLWNKLDTKRQELMLNEMNTMLQEPNISNNLKEYLLRLTNKL

SEQ ID NO: 2 - protein, 1064 aa, Plasmodium berghei
MVLKKLLCFNLFLIIILTFENLSFDKKNTCMINNTIRSNSCCIVNRVLREKTHHYSSKISKSIPFIQNFSLEKYFTGESLQKNILNNINKLGGAHLFHISKSHLTAKSGNKNTEFIGEATELFKGFKRNFGINMTENKQTNIGRMLCENDNNNGGEDTSTEKAIFKKSKDSQIHYRTDYKPSGFTIDNVTLNINIFDNETIVRSSLNMCTNENYADEDLVFDGVGLSIKEISINNNKLTEGEDYTYDNEFLTIFAKNVPKENFVFLSEVVIHPETNYALTGLYKSKDIIVSQCEATGFRRITFFIDRPDMMAKYDVTLTADKKKYPVLLSNGDKLNEFDIPGGRHGARFNDPHLKPCYLFAVVAGDLKHLSDNYVTKYTKKPVELYVYSEAKYVSKLKWALECLKKAMKFDEDYFGLEYDLSRLNLVAVSDFNVGAMENKGLNIFNADSLLASKKTSIDFSFERILTVVGHEYFHNYTGNRVTLRDWFQLTLKEGLTVHRENLFSEETTKTATFRLTHIDLLRSVQFLEDSSPLSHPIRPESYISMENFYTNTVYDKGSEVMRMYQTILGDDYYKKGIDIYLKKHDGGTATCEDFNDAMNEAYQMKKGNTDENLDQYLLWFSQSGTPHVTAEYIYDENEKTFTINLSQITYPDDNQKEKYPLFIPVKVGFISPKDGKDVIPETVLELKKDKESFVFQNVSEKPIPSLFREFSAPVYIKDNLTDEERIALLKYDSDAFVRYNVCIDLYMKQIIKNYNELISQKTKENNVLELSLTPVNDEFINAIKHLLEDKHADPGFKSYIIALPRDRYIMNYIKEVDPIVLADTKDYIYKQIGSRLNPVLFSIFQNTESKANDMTHFKDESYIDFDQLNMRKLRNSILMMLSKAQYPHMLKYIKEQSNSPYPSNWLASLSASSYFSGDDYYDLYDKTYKLSKNDELLLQEWLKTVSRSDRSDIYSIIKKLEVEILKDSKNPNNIRAVYLPFTSNLRAFNDISGKGYKLMANVIMKVDKFNPMVATQLCDPFKLWNKLDLKRQALMHDEMNRMLNMENISPNLKEYLLRLTNKM

SEQ ID NO: 3 - protein, 1064 aa, Plasmodium yoelii yoelii
MVTKKLLSFNLFLIIILTFENLTFDKKNTCMINNTIRPNSCGIVNRVLREKPYHYSSKISKSIPFIQNFSLEKNFTGESLQKNILNNINKLGGAHLFHISKSHLAAKAGNKNTEFIGEATELFKGFKRNFGINMTENKQTNIGRMLCEDDNNNGGEVTSTEKTIFKNSKDPQIHYRTDYKPSGFTIDNVTLNINIFDNETIVRSSLNMCTNENYADEDLVFDGVGLSIKEISINNNKLNEGEDYTYDNEFLTIFAKNVPKENFVFLSEVVIHPETNYALTGLYKSKDIIVSQCEATGFRRITFFIDRPDMMAKYDVTLTADKTKYPVLLSNGDKLNEFDIPGGRHGARFNDPHLKPCYLFAVVAGDLKHLSDNYVTKYTKKPVELYVYSEAKYVSKLKWALECLKKAMKFDEDYFGLEYDLSRLNLVAVSDFNVGAMENKGLNIFNADSLLASKKTSIDFSFERILTVVGHEYFHNYTGNRVTLRDWFQLTLKEGLTVHRENLFSEETTKTATFRLTHIDLLRSVQFLEDSSPLSHPIRPESYISMENFYTNTVYDKGSEVMRMYQTILGDEYYKKGIDIYLKKHDGGTATCEDFNDAMNEAYQMKNGNTDENLDQYLLWFSQSGTPHVTAEYIYDENEKTFTINLSQITYPDDNQKEKYPLFIPVKVGFISPKDGKDVIPEVVLELKKDKESFVFQNVSEKPIPSLFREFSAPVYIKDNLTDEERIALLKYDSDAFVRYNVCIDLYMKQIIKNYNELVSQKTKENDLVELSLTPVNDEFINAIKHLLEDKHADPGFKAYIIALPRDRYIMNYIKEVDPIVLADTKDYIYKQIGSRLNPILFSIFQNTESKANDMTHFKDESYIDFDQLNMRKLRNSILVMLSKAQYPHMLKYIKEQSKSAYPSNWLASLSASAYFSGDDYYDLYDKTYKLSKNDELLLQEWLKTVSRSDRSDIYSIIKKLEVEILKDSKNPNNIRAVYLPFTANLRAFNDISGKGYKLMADVIMKVDKFNPMVATQLCDPFKLWNKLDLKRQALMHDEMNRMLSMENISPNLKEYLLRLTNKM

SEQ ID NO: 4 - protein, 895 aa, Plasmodium chaubaudi
DPQIHYRTDYKPSGFTIDTVTLNINIFDNETTVRSSLSMCTNDNYANEDLVFDGVGLSIKEININDNKLTEGEDYTYDNEFLTVFAKNVPKGNFVFVSEVIIHPETNYALTGLYKSKDIIVSQCEATGFRRITFFIDRPDMMAKYDVTLTADKTKYPVLLSNGDKLNEFDIPGGRHGARFNDPHLKPCYLFAVVAGDLKFLSDKYVTKFTKKPVELYVYSEEKYVSKLKWALECLKKAMKFDEDYFGLEYDLSRLNLVAVSDFNVGAMENKGLNIFNADSLLASKKTSIDFSFERILTVVGHEYFHNYTGNRVTLRDWFQLTLKEGLTVHRENLFSEQTTKTATFRLTHIDILRSVQFLEDSSPLSHPIRPESYISMENFYTNTVYDKGSEVMRMYQTILGDEYYKKGISIYLKKHDGGTATCEDFNEAMNEAYQMKNGNKEENLDQYLLWFSQSGTPHVTAEYSYDANAKTFTIKLSQVTYPDDNQKEKFPLFIPVKVGLISPKDGKDVIPEVVLEFKKDKDTFVFENIEEKPIPSLFREFSAPVYIKDNLTDEERIILLKYDSDAFVRYNVCIDLYMKQIIKNYNEFLSQKTKEANGLEHSLTPVSEDFINAIKHLLEDKHSDPGFKAYIIALPRDRYIMNYIKEVDPIILADTKDYIYKQMGNRLNPILFSIFQDTESKANDMTHFKDESYVDFDQLNMRKLRNSIMVMLSKAQYPHMLKYVKDQAQSPYPSNWLASLSASAYFTGDDYYNLYDKTYNLSKNDELLLQEWLKTVSRSDRSDIYNIIKKLETEILKDSKNPNNIRAVYLPFTSNLRAFNDISGKGYKLMADVIMKVDKFNPMVATQLCDPFKLWNKLDLKRQALMHDEMNRMLSMDNISPNLKEYLLRLTNKM

SEQ ID NO: 5 - protein, 1097 aa, Plasmodium vivax
MIRKKMLNFHFLFITVLVALANYTPVDYQNTCMISKSCRKNSCGITSRVLSGIHVNKTSARAKALISLSSLIYHLQLPKLVSLDLFRRDLFTGVKQKGKRSPVPSYIIQNRLMSENGDSGSTNMSVTANQEKKRPGTGDASEGNNQSGISAAAQDKRMKGGDQSEEVSNVSGSTNAAMTNGASSTTEGGDNNNNGSGNDGKNEPKIHYRKDYKPSGFVIDNVTLNINIFDNETSVRSTLDMKLSEHYGGEDLIFDGVSLEIKEISIDNNKLMEGEHYKYDNEFLTIYSKFIPKGKFTFGSEVIIHPETNYALTGLYKSKNIIVSQCEATGFRRITFFIDRPDMMAKYDVTITADKEKYPVLLSNGDKLNEFEIPGGRHGARFNDPYLKPCYLFAVVAGDLKHLSDNYVTKFSKKNVELYVFSEEKYVSKLKWALECLKKAMKFDEDYFGLEYDLSRLNLVAVSDFNVGAMENKGLNIFNANSLLASKKKSIDFSFERILTVVGHEYFHNYTGNRVTLRDWFQLTLKEGLTVHRENLFSEQTTKTATFRLDHVDILRSVQFLEDSSPLAHPIRPESYVSMENFYTTTVYDKGSEVMRMYQTILGDEYYKKGMDIYIKKNDGGTATCEDFNDAMNEAYKMKKGDKTANLDQYLLWFSQSGTPHVTAEYSYDAGKKEFVIEVTQVTNPDPNQKEKKALFIPIRVGFINPKNGQDVIPEVTLEFKKDKEKFIFNNVNEKPIPSLFRGFSAPVYIKDNLTDSERILLLKYDTDAFVRYNVCVDLYMKQILKNYQELLQAKSENKQESAEKPSLTPVSEDFINAIKYLMEDPHADAGFKSYIITLPRDRFILNYIKNVDTDVLADTKDFIYKQLGDKLNDLYFQMFKSLQAKADDMTHFEDESYVDFEQLNMRKLRNTLLTLLSRAKYPNMLDQIMEHSKSPYPSNWLASLAVSAYYDKYFDLYEKTYNQSKDDELLLQEWLKTVSRSDRKDIYDIIKKLETEVLKDSKNPNEIRAVYLPFTYNLRYFNDISGKGYKMMADIIMKVDKFNPMVATQLCDPFKLWNKLDQKRQDMMLNEMNRMLSMENISNNLKEYLLRLTNKL

SEQ ID NO: 6 - protein, 1096 aa, Plasmodium knowlesi
MIRERMLNLYFLFITVLAALAIYTPVDYQNTCMISRSCRKNSCGITSRVLSGIHVNKASTRARALISLSSLIYQLQLPKLVSLDLFRRDLFTGVNQKGRRSPVPSYIIQSRLMSENIDSGNNNMSATGNQEKKRPVTGDASDAKNPDGSTISAQDKRMKGIDQNEGTSSVSGSTNAAFTNGASSIMEGGENNNNGTGNEGKNEPTIHYRKDYRPSGFIIDNVTLNINIFDNETSVRSTLDKLSDHYRGEDLIFDGVSLEIKEISIDGNKLMEGEHYKYDKEFLTIYSKFIPKGKFTFGSEVIIHPETNYALTGLYKSKNIIVSQCEATGFRRITFFIDRPDMMAKYDVTVTADKEKYPVLLSNGDKLNEFEIPGGRHGARFNDPYLKPCYLFAVVAGDLKHLSDNYVTKFSKRNVELYVFSEEKYVSKLKWALECLKKAMKFDEDYFGLEYDLSRLNLVAVSDFNVGAMENKGLNIFNANSLLASKKKSIDFSFERILTVVGHEYFHNYTGNRVTLRDWFQLTLKEGLTVHRENLFSEQTTKTATFRLDHVDLLRSVQFLEDSSPLAHPIRPESYVSMENFYTTTVYDKGSEVMRMYQTILGDDYYKKGMDIYIKKNDGGTATCEDFNDAMNEAYKLKKGDKTANLDQYLLWFAQSGTPHVTAEYSYDAGKKEFVIDITQVTHPDPNQKEKKALFIPIRVGFINPHNGKEVIPEVTLEFKKDKEKFIFSNVNEKPIPSLFRGFSAPVYIKDNLTDSERIVLLKYDTDAFVRYNVCVDLYMKQIMKNYQELLQAKAENKQESTEKPKLTPVSEDFISAIKYLMEDPHADAGFKSYIITLPRDRFIINSIRNVDTDVLADTKDFIYKQLGDKLNDLYFQIFKSIQAKADDMTHFEDESYVDFEQLNMRKLRNTLLTLLSKAKYPNMLDHIMEHSKSPYPSNWLASLAVSAYYDKYFDLYEKTYNQSKDDELLLQEWLKTVSRSDRKDIYDIIKKLENEVLKDSKNPNEIRAVYLPFTNNLRYFNDISGKGYKMMADIIMKVDKFNPMVATQLCEPFKLWNKLDMKRQDMMLNEMNRMLSMENISNNLKEYLLRLTNKL

(19) United States
(12) Patent Application Publication
(10) Pub. No.: US 2011/0311511 A1
(43) Pub. Date: Dec. 22, 2011

(54) CRYSTAL STRUCTURE OF PfA-M1 AND THE PfA-M1 Co4 COMPLEX

(76) Inventors: James Charles Whisstock, Murrumbeena (AU); Ashley Maurice Buckle, Mt. Dandenong (AU); Sheena McGowan, Berwick (AU); Corrine Joy Porter, Caulfield North (AU); John P. Dalton, Ultimo Sydney (AU); Jonathon Lowther, Edinburgh (GB); Colin Martin Stack, Douglas Park (AU); Sheila Mary Donnelly, Ultimo Sydney (AU)

(21) Appl. No.: 12/867,730

(22) PCT Filed: Feb. 12, 2009
(86) PCT No.: PCT/AU2009/000161

§ 371 Date: Apr. 8, 2011

(30) Foreign Application Priority Data
Feb. 14, 2008 (AU) 2008900700

(51) Int. Cl. A61K 38/46; C12Q 1/37; G06G 7/60; C40B 30/02; G06F 17/30; C12N 9/50; A61P 33/00

(52) U.S. Cl. 424/94.67; 435/219; 435/23; 506/8; 707/792; 703/11

(57) ABSTRACT

The invention relates to the X-ray crystal structure of PfA-M1 aminopeptidase alone, and in complex with the phosphinate dipeptide analogue hPheP[CH₂]Phe. More specifically the present invention provides the structure coordinates of PfA-M1 and PfA-M1 in complex with Co4. The invention also includes the use of the X-ray crystal structures as drug target models for anti-malarial drug design and a method for identifying or designing novel anti-malarial drugs, for example using high-throughput chemical screening and medicinal chemistry methods. The invention further provides anti-malarial drugs identified or designed according to the aforementioned method and their use for obstructing protein metabolism and synthesis in a parasite by blocking the entrance of Hb-derived peptides and/or blocking the exit of released amino acids at the active site of PfA-M1 protease.

### FIELD OF THE INVENTION

The invention relates to the X-ray crystal structure of PfA-M1 aminopeptidase alone, and in complex with the phosphinate dipeptide analogue hPheP[CH₂]Phe. The present invention further relates to the use of the X-ray crystal structures as drug target models for anti-malarial drug design.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

There are 300-500 million cases of clinical malaria annually, and 1.4-2.6 million deaths. Malaria is caused by parasites of the genus Plasmodium, with Plasmodium falciparum the most lethal of the four species that infect humans. Clinical manifestations begin when parasites enter host erythrocytes and most anti-malaria drugs, such as chloroquine, exert their action by preventing the parasite development within these cells (Rosenthal, P. J. J. Exp. Biol. 206, 3735-3744 (2003)). Intra-erythrocytic parasites have limited capacity for de novo amino acid synthesis and rely on degradation of host haemoglobin to maintain protein metabolism and synthesis (Rosenthal, P. J. J. Exp. Biol. 206, 3735-3744 (2003); Liu, J., Istvan, E. S., Gluzman, I. Y., Gross, J. & Goldberg, D. E. Proc Natl Acad Sci USA 103, 8840-5 (2006)).

Haemoglobin (Hb) is initially degraded by endoproteases within a digestive vacuole (DV) to di- and tri-peptide fragments (Klemba, M., Gluzman, I. & Goldberg, D. E. J Biol Chem 279, 43000-7 (2004); Rosenthal, P. J. Curr Opin Hematol 9, 140-5 (2002)) that are then exported to the parasite cytoplasm (Curley, G. P. et al. J Eukaryot Microbiol 41, 119-23 (1994); Kolakovich, K. A., Gluzman, I. Y., Duffin, K. L. & Goldberg, D. E. Mol Biochem Parasitol 87, 123-35 (1997)) (FIG. 3).

Release of amino acids involves two metallo-exopeptidases; an alanyl aminopeptidase, PfA-M1, and a leucine aminopeptidase PfA-M17 (Curley, G. P. et al. J Eukaryot Microbiol 41, 119-23 (1994); Allary, M., Schrevel, J. & Florent, I. Parasitology 125, 1-10 (2002); Gavigan, C. S., Dalton, J. P. & Bell, A. Mol Biochem Parasitol 117, 37-48 (2001); Stack, C. M. et al. J Biol Chem 282, 2069-80 (2007)). Phosphinate dipeptide analogues that inhibit metallo-aminopeptidases prevent the growth of wild-type and the chloroquine-resistant parasites in culture and one compound, hPheP[CH₂]Phe (termed Compound 4, Co4), reduced a murine infection of P. c. chabaudi by 92% compared to controls (Grembecka, J., Mucha, A., Cierpicki, T. & Kafarski, P. J Med Chem 46, 2641-55 (2003); Skinner-Adams, T. S. et al. J. Med. Chem. 50, 6024-6031 (2007)).

There is a large number of different anti-malaria drugs available. They all have different modes of action and different side effects. No currently available malarial drug is 100% effective in preventing malaria and some are not effective in certain parts of the world. Accordingly, there is a great deal of scope for improving anti-malarial medication.

Irrespective of this, there is a paucity of new anti-malarial drugs entering the development pipeline. Modern drug development focuses on the development of drug targets, that is, genes or cellular chemicals that are associated with a specific disease. In the field of anti-malarial drugs there is a need for development of viable, validated drug target models. In particular there is a need for model structures and structural data that can facilitate the design of drugs that can inhibit malarial parasites.

It has now been found that malaria neutral aminopeptidase, PfA-M1, can be functionally characterised and validated as a drug target.

### SUMMARY OF THE INVENTION

The present invention provides functional characterisation of PfA-M1 in terms of its three-dimensional structure alone and in complex with Co4.

### Crystal Structure

The present invention therefore provides the structure coordinates of PfA-M1. The complete coordinates are listed in Table A.

The present invention further provides the structure coordinates of PfA-M1 in complex with Co4. The complete coordinates are listed in Table B.

The present invention further provides a crystal of PfA-M1 consisting of a primitive orthorhombic P2₁2₁2₁ space group with unit cell dimensions of a=75.7±2.1 Å, b=108.7±2.1 Å and c=118.0±2.1 Å.

The present invention further provides a crystal of PfA-M1 in complex with Co4 consisting of a primitive orthorhombic P2₁2₁2₁ space group with unit cell dimensions of a=75.9±2.0 Å, b=108.6±2.0 Å and c=118.3±2.0 Å.

The present invention also provides a machine-readable data storage medium which comprises a data storage material encoded with machine readable data defined by the structure coordinates of PfA-M1 according to Table A or a homologue of this structure.

The present invention also provides a machine-readable data storage medium which comprises a data storage material encoded with machine readable data defined by the structure coordinates of PfA-M1 in complex with Co4 according to Table B or a homologue of this structure

The present invention thus provides a structural model for the unique active site structure of PfA-M1 alone, and in complex with the anti-malarial Co4. The use of the PfA-M1 structural model and the use of the PfA-M1 Co4 complex structural model have been validated. The structural model, having been validated, can be used for the identification of novel class of anti-malarials using high-throughput chemical screening and medicinal chemistry methods.

While PfA-M1 functions in the terminal stages of haemoglobin digestion releasing amino acids essential for parasite protein anabolism, Co4 also inhibits the second important neutral aminopeptidase of malaria, PfA-M17 (Stack, C. M. et al. J Biol Chem 282, 2069-80 (2007); Gardiner, D. L., Trenholme, K. R., Skinner-Adams, T. S., Stack, C. M. & Dalton, J. P. J Biol. Chem. 281, 1741-5 (2006)). Thus the structural model of the PfA-M1 and Co4 complex of the present invention provides a useful tool for development of a two-target or combination therapy that would be more resilient to the emergence of drug resistant malaria parasites. The structure of PfA-M1 reveals two openings to the active site cavity. Analysis of the Co4-bound rPfA-M1 structure revealed that it is essentially identical to the inhibitor-free enzyme.

Accordingly, the present invention also provides a method for determining at least a portion of the three-dimensional structure of a species, such as a molecule or molecular complex which can bind with the active site, or the active site cavity. The molecule or molecular complex may for example stabilise, alter the conformation of, or interact with the active site or active site cavity. It is preferred that these molecules or molecular complexes correspond to at least part of the active binding site defined by structure coordinates of rPfA-M1 according to Table A or the Co4-bound PfA-M1 according to Table B.

The present invention further provides a method for screening molecules or molecular complexes for anti-malarial activity comprising the steps of:
(i) characterising the active site cavity from the structure coordinates of Table A or Table B;
(ii) identifying candidate molecules or molecular complexes that interact with at least part of the active site cavity; and
(iii) obtaining or synthesizing said candidate molecule or molecular complex.

The part of the active site cavity with which the candidate compound interacts is typically the C-terminal domain IV opening, the groove at the junction of domains I and IV or the active site. Our interpretation is that the larger C-terminal channel is the entrance whereby Hb-derived peptides access the buried active site leaving the smaller sized opening for exit of released amino acids. Accordingly the candidate molecule or molecular complex will block the entrance of Hb-derived peptides to the buried active site, and/or block the exit of released amino acids.

One of the advantages of using a structure based model as a drug target is that it has a high degree of specificity, that is, the model makes it possible to choose or design a molecule or molecular complex that blocks the PfA-M1 protease, but does not adversely affect other proteases that may be beneficial, or essential to a host.

The present invention further provides a method for screening molecules or molecular complexes for anti-malarial activity comprising the steps of:
(i) characterising the active site from the structure coordinates of Table A or Table B;
(ii) identifying candidate molecules or molecular complexes that interact with one or more of the following amino acids: Ala₃₂₀, Ala₄₆₁, Arg₄₈₉, Gln₃₁₇, Glu₃₁₉, Glu₄₆₃, Glu₆₁₆, Glu₄₉₇, Glu₄₆₀, His₄₉₆, His₅₀₀, Lys₆₁₈, Met₄₆₂, Met₁₀₃₄, Thr₄₉₂, Tyr₅₇₅, Tyr₅₈₀, Val₄₅₉ and Val₄₉₃,
(iii) obtaining or synthesizing said candidate molecule or molecular complex.

In a particularly preferred embodiment step (ii) consists of identifying candidate molecules or molecular complexes that interact with one or more of the following (inclusively numbered) residues that line the active site of the malaria protease: 303-305; 314-325; 458-463; 489-526 (incorporating ‘catalytic residues’ His-496; His-500 and Glu-519); 570-582; and 1022-1038.

The present invention further provides an active binding site or active binding site cavity in rPfA-M1 or the Co4-bound rPfA-M1 structure as well as methods for designing or selecting molecules or molecular complexes for use as anti-malarial drugs using information about the crystal structures disclosed herein. The present invention further provides anti-malarial drugs or drug candidates designed or selected according to said method.

In a preferred embodiment the methods, drugs or drug candidates of the present invention are suitable for modulating PfA-M1 or the Co4-bound PfA-M1 complex to inhibit at least part of their activity, more preferably all of their activity. In a particularly preferred embodiment, the inhibition will stop degradation of haemoglobin. In situ this means that the parasite from which the protease originated will be deprived of materials to maintain protein metabolism and synthesis.

### BRIEF DESCRIPTION OF THE DRAWINGS

Various embodiments/aspects of the invention will now be described with reference to the following drawings in which,

FIG. 1:

FIG. 1(A): Western blot of transgenic parasites expressing the product of the inserted transgene encoding PfA-M1. The blot was probed with a monoclonal anti-c-myc primary antibody followed by horseradish peroxidase anti-mouse immunoglobulin antibodies and visualised by enhance chemiluminescence.

FIG. 1(B): Indirect immunofluorescence of transgenic parasites stained with monoclonal anti-c-myc primary antibody followed by anti-mouse cy2. (i) bright field; (ii) anti-c-myc antibody; (iii) anti-c-myc/nuclear stain merged; (iv) merge of (i) and (ii). The data show that the PfA-M1 transgenic protein is localized to the parasite cytosol.

FIG. 1(C): Northern blot analysis of stage-specific parasite RNA reveals that the endogenous PfA-M1 is expressed by parasites at all developmental stages within the erythrocyte.

FIG. 1(D): The pH optima for activity of rPfA-M1 (circles) and native PfA-M1 in soluble extracts of parasites (squares) measured by fluorogenic peptides substrate H-Arg-NHMec.

FIG. 2:

FIG. 2(A): Cartoon of Co4-bound rPfA-M1 coloured by domain: I (blue), II (green), III (yellow), (IV) red and Co4 shown as sticks (inside catalytic domain II).

FIG. 2(B): Molecular surface diagram (coloured as A) showing small opening to active site (magenta), respectively.

FIG. 2(C): Molecular surface diagram of large cavity opening to active site formed by domain IV.

FIG. 2(D): Electrostatic potential surface of active site containing Co4. Domain IV is excluded for clarity. The colour of the surface represents the electrostatic potential at the protein surface, going from black (potential of +10 kT/e) to grey (potential of −10 kT/e), where T is temperature, e is the charge of an electron, and k is the Boltzmann constant.

FIG. 2(E): Binding of Co4 to active site of PfA-M1. Atom numbers of Co4 are indicated. Zinc ion is shown as solid black sphere. Water molecules are shown as small grey spheres. Hydrogen bonds between Co4 and PfA-M1 are shown as dashed lines. Residues of PfA-M1 active site are labelled.

FIG. 3: Flow diagram of how a digestive vacuole protease degrades haemoglobin.

FIG. 4: Chart showing alignment of Plasmodium spp. M1 neutral aminopeptidases. Sequence alignment was prepared using ClustalW and Espript. Identical residues are shaded. The Plasmodium spp. are listed on the left. Numbering as per PfA-M1. GAMEN substrate recognition motif is boxed and zinc binding motif underlined with catalytic residues indicated by an astrix (*). Truncated rPfA-M1 start amino acid is circled.

FIG. 5: Electron density of Co4 binding to the active site of rPfA-M1. The composite omit map was contoured at 1.0 sigma without consideration of the structure factors of Co4 or zinc.

### DETAILED DESCRIPTION OF THE INVENTION

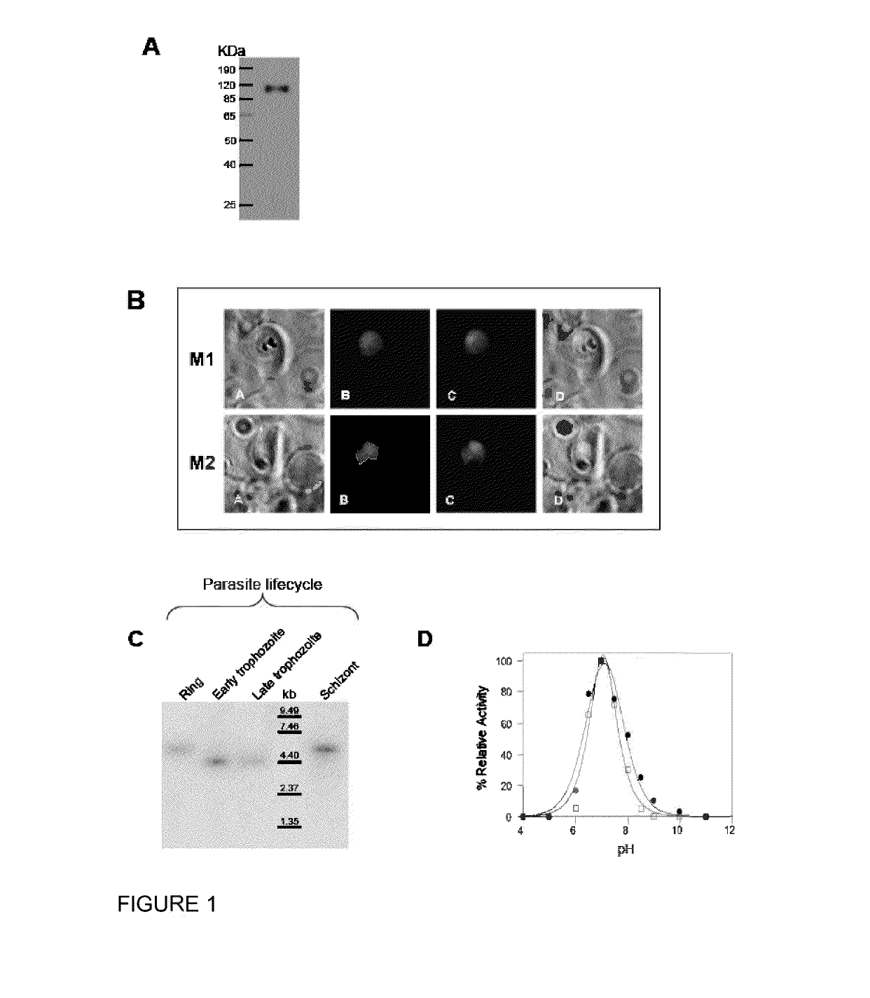

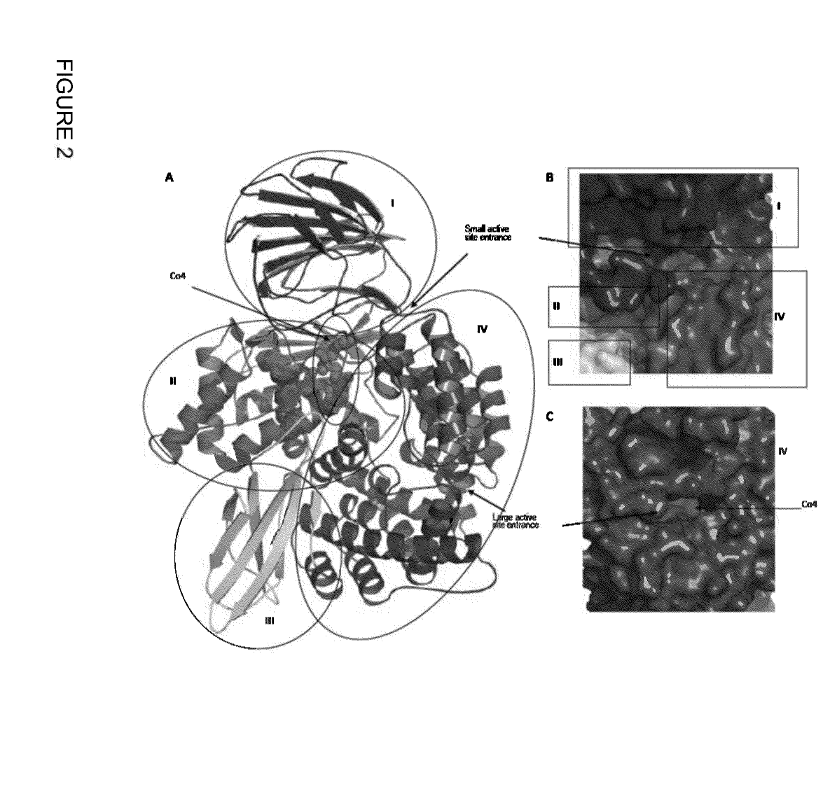

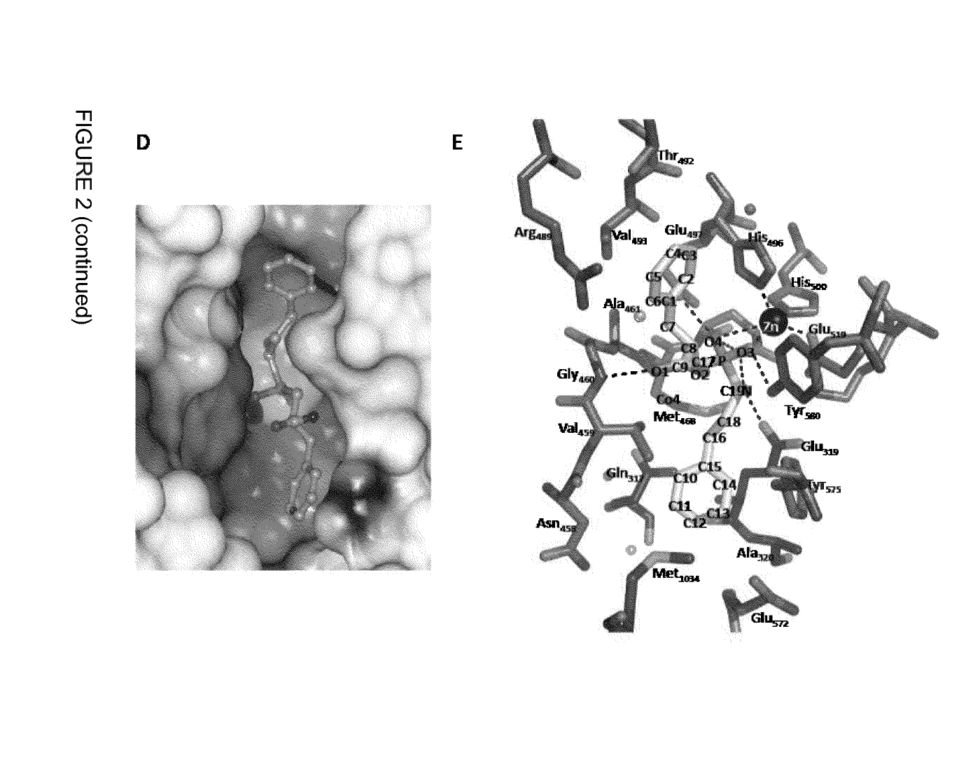

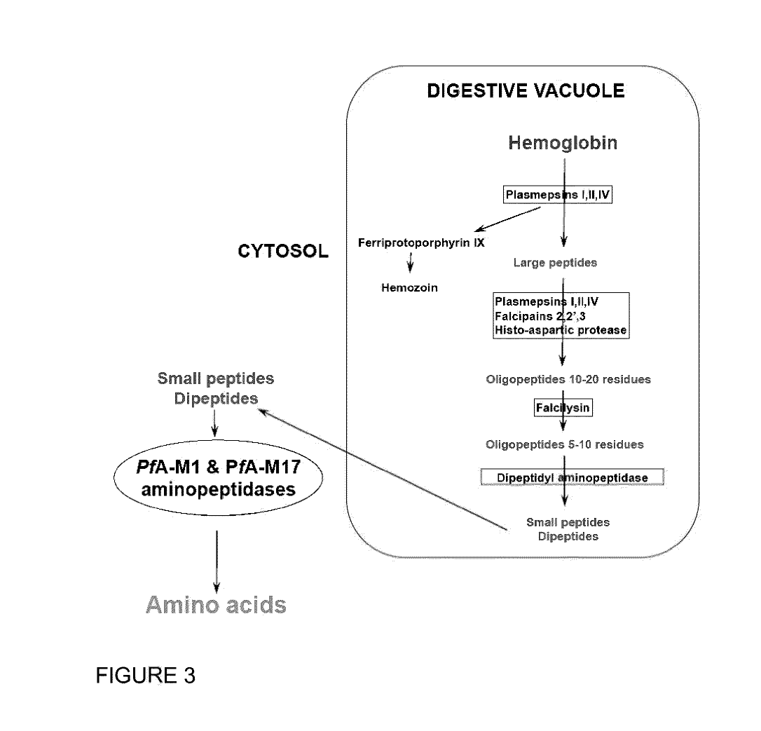

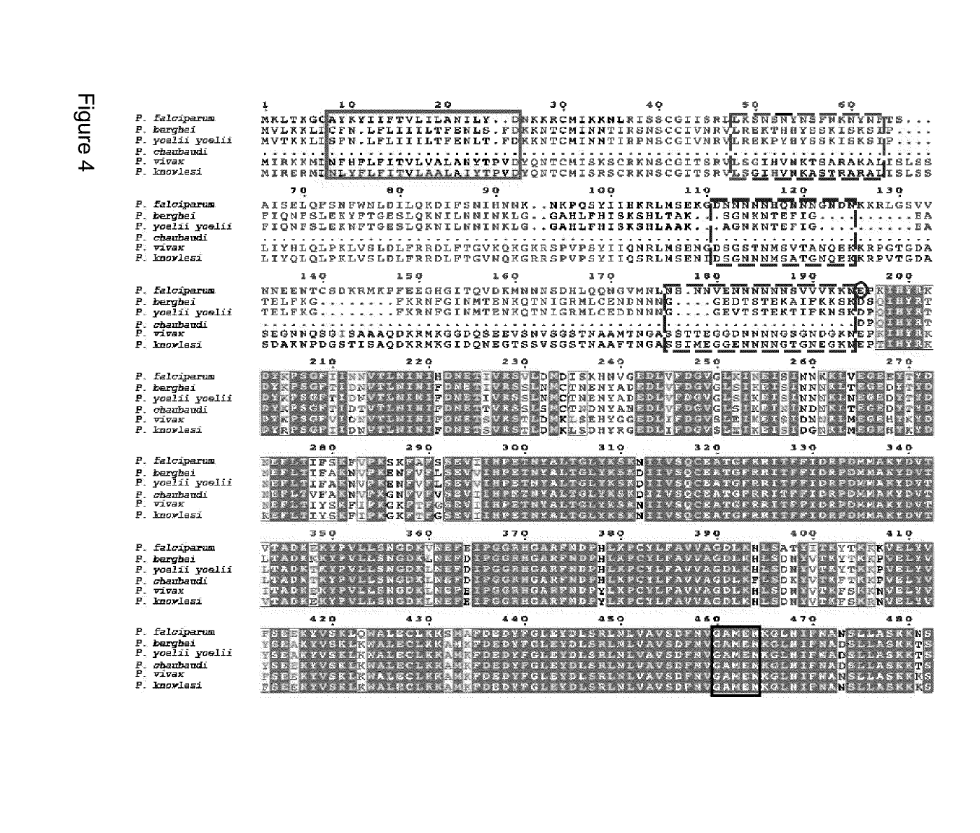

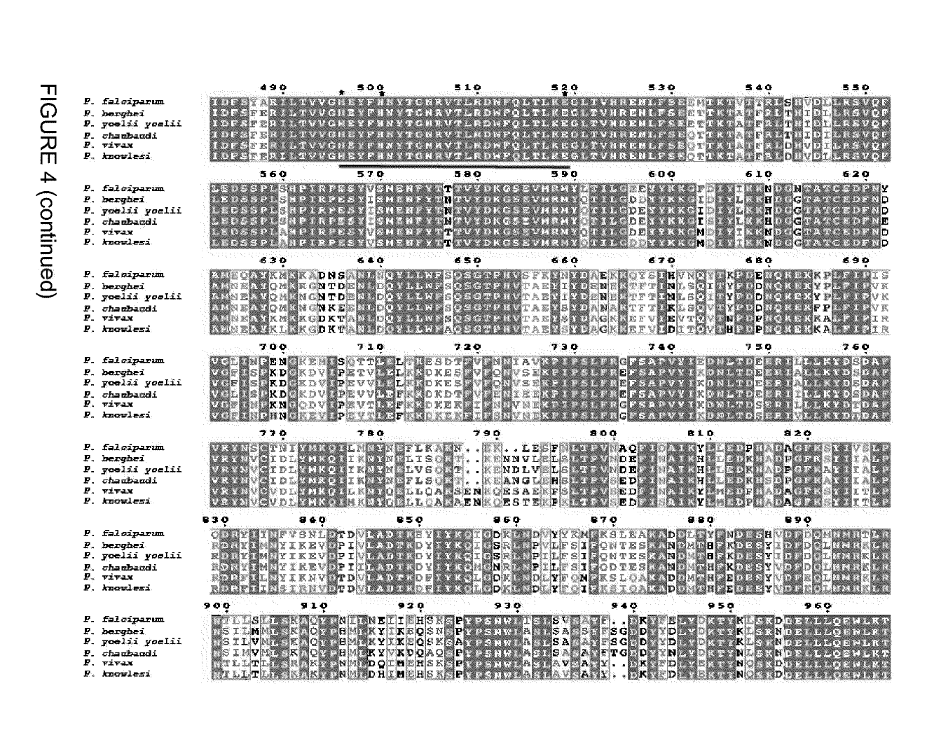

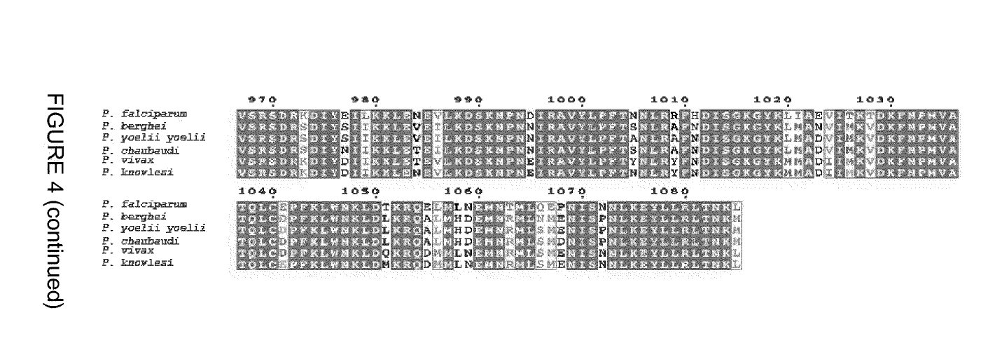

PfA-M1 is a 1085 residue metallo-exoprotease, highly conserved between different Plasmodium spp. (FIG. 4) and is expressed by all intra-erythrocytic developmental stages (FIG. 10, Florent, I. et al. Mol Biochem Parasitol 97, 149-60 (1998)). P. falciparum D10 parasites transfected with the plasmid pHTB-PfA-M1-cmycB expressed a product of ˜115 kDa (FIG. 1A) within the parasite cytosol (FIG. 1B). These transgenic parasites expressed a 2.8-fold higher level of alanyl aminopeptidase activity compared to D10 wild-type parasites showing that the transgene product was functionally active within the parasite.

TABLE 1
Comparison of the specificity constants for various N-terminal amino acids
for recombinant P. falciparum M1 aminopeptidase (rPfA-M1) at pH 7.5

| | | | K_{cat}/Kₘ | Abundance in |
|---|---|---|---|---|
| Substrate | k_{cat} (s⁻¹) | Kₘ (μM) | (M⁻¹s⁻¹) | human Hb (%) |
| H-Leu-NHmec | 1.52 | 329.9 | 4607 | 12.46 |
| H-Ala-NHmec | 2.04 | 888.9 | 2295 | 12.46 |
| H-Arg-NHmec | 1.07 | 717.4 | 1491 | 2.08 |
| H-Phe-NHmec | 0.18 | 194.8 | 924 | 5.19 |
| H-Gly-NHmec | 0.116 | 348.6 | 333 | 6.92 |
| H-Val-NHmec | 0.036 | 1068.1 | 34 | 10.73 |
| H-Ile-NHmec | 0.040 | 1706 | 23 | 0 |
| H-Pro-NHmec | 0.0032 | 734.4 | 4 | 4.84 |

Recombinant PfA-M1 (rPfA-M1) displayed a broad specificity, cleaving N-terminal hydrophobic, basic, and aromatic amino acids (Table 1).

The most efficiently cleaved residues were (at the P1 position) Leu, Ala, Arg and Phe that represent 32% of haemoglobin residues (Table 1). rPfA-M1 displayed optimal activity at pH 7.0 with <20% activity below pH 6.0, similar to alanyl aminopeptidases activity within soluble extracts of malaria parasites, and consistent with a function within the cytosol (FIG. 1D and Stack, C. M. et al. J Biol Chem 282, 2069-80 (2007)). Co4 was a potent inhibitor of the rPfA-M1 (Kᵢ=78.35 nmolar).

The X-ray crystal structures of the ligand-free and Co4-bound rPfA-M1 were determined to 2.1 Å and 2.0 Å, respectively (see Table 2 and the methods set out in the Examples).

TABLE 2
Data Collection and refinement statistics

| | |
|---|---|
| rPfA-M1 | rPfA-M1-Co4 |

Data collection

| Space Group | P2₁2₁2₁ | | P2₁2₁2₁ | |
|---|---|---|---|---|
| Cell dimensions (Å) | a = | 75.7, | a = | 75.9, |
| | b = | 108.7, | b = | 108.6, |
| | c = | 118.4, | c = | 118.3 |

| | | |
|---|---|---|
| Resolution (Å) | 34.99-2.1 (2.21-2.1) | 28.61-2.0 (2.11-2.0) |
| Total number of | 289352 | 432263 |
| observations | | |
| Number of unique | 56863 | 60523 |
| observations | | |
| Multiplicity | 5.1 (3.8) | 7.1 (5.5) |
| Data Completeness (%) | 98.7 (94.0) | 91.0 (77.5) |
| <I/σ_{I}> | 16.7 (2.8) | 22.0 (3.0) |
| Rₚᵢₘ (%)^{b} | 4.2 (21.3) | 3.0 (24.5) |

Structure refinement

| | | |
|---|---|---|
| Non hydrogen atoms | | |
| Protein | 7233 | 7332 |
| Solvent | 762 | 739 |
| Ligand | — | 26 |
| R_{free} (%) | 22.0 | 22.1 |
| R_{cryst} (%) | 17.0 | 17.5 |
| Rms deviations from | | |
| ideality | | |
| Bond lengths (Å) | 0.010 | 0.009 |
| Bond angles (°) | 1.13 | 1.33 |
| Ramachandran plot | | |
| Favoured (%) | 98.0 | 98.2 |
| Allowed (%) | 100 | 100 |
| B factors (Å²) | | |
| Mean main chain | 21.8 | 17.4 |
| Mean side chain | 22.6 | 19.8 |
| Mean ligand | — | 23.5 |
| Mean water molecule | 30.8 | 33.3 |
| r.m.s.d. bonded Bs | | |
| Main chain | 0.48 | 0.77 |
| Side chain | 1.1 | 2.3 |
| MolProbity Score (44) | 1.38 (99^{th} percentile^{c}) | 1.45 (98^{th} percentile^{c}) |

^{a}Values in parentheses refer to the highest resolution shell.
^{b}Agreement between intensities of repeated measurements of the same reflections and can be defined as: Σ(I_{h, i} − <Iₕ>)/Σ I_{h, i}, where I_{h, i} are individual values and <Iₕ> is the mean value of the intensity of reflection h.
^{c}N = 12522, 1.75 Å-2.25 Å (44)
^{d}N = 9033, 1.4 Å-1.9 Å (44)
rPfA-M1 adopts the bacterial aminopeptidase N fold (Addlagatta, A., Gay, L. & Matthews, B. W. Proc Natl Acad Sci USA 103, 13339-44 (2006); Ito, K. et al. J Biol Chem 281, 33664-76 (2006)), and comprises 26 α-helices and 7 β-sheets divided into four domains (FIG. 2A). The catalytic domain II (residues 392-649) adopts a thermolysin-like fold and contains the active site, incorporating the zinc-binding motif H₄₉₆EYFHX₁₇KE₅₁₉ and the well-conserved G₄₆₀AMEN motif involved in substrate recognition (Addlagatta, A., Gay, L. & Matthews, B. W. Proc Natl Acad Sci USA 103, 13339-44 (2006); Ito, K. et al. J Biol Chem 281, 33664-76 (2006)). The catalytic zinc ion is coordinated by Nε2 atoms of His₄₉₆ and His₅₀₀, the carboxyl Oε atom of Glu₅₁₉, and a water molecule in the ligand free form.

### Structural Characteristics

Inspection of the molecular surface of PfA-M1 reveals two openings to the active site cavity. The first opening comprises a shallow 8 Å long groove at the junction of domains I and IV (FIG. 2B). The second and larger opening is formed by the C-terminal domain IV, which comprises eight pairs of α-helices arranged in two layers to form a cone-shaped superhelical structure. This domain interacts with the catalytic domain II and contains a ˜28 Å long channel leading towards the active site (FIG. 2C). At the entrance is a helix (α₁₄) with a 90° bend that confines the pore size to approximately 15 Å diameter. This is notably larger than observed in bacterial homologs (Addlagatta, A., Gay, L. & Matthews, B. W. Proc Natl Acad Sci USA 103, 13339-44 (2006); Ito, K. et al. J Biol Chem 281, 33664-76 (2006)) indicating a more open conformation of the active site in the malarial protease. Our interpretation is that the larger C-terminal channel is the entrance whereby Hb-derived peptides access the buried active site leaving the smaller sized opening for exit of released amino acids.

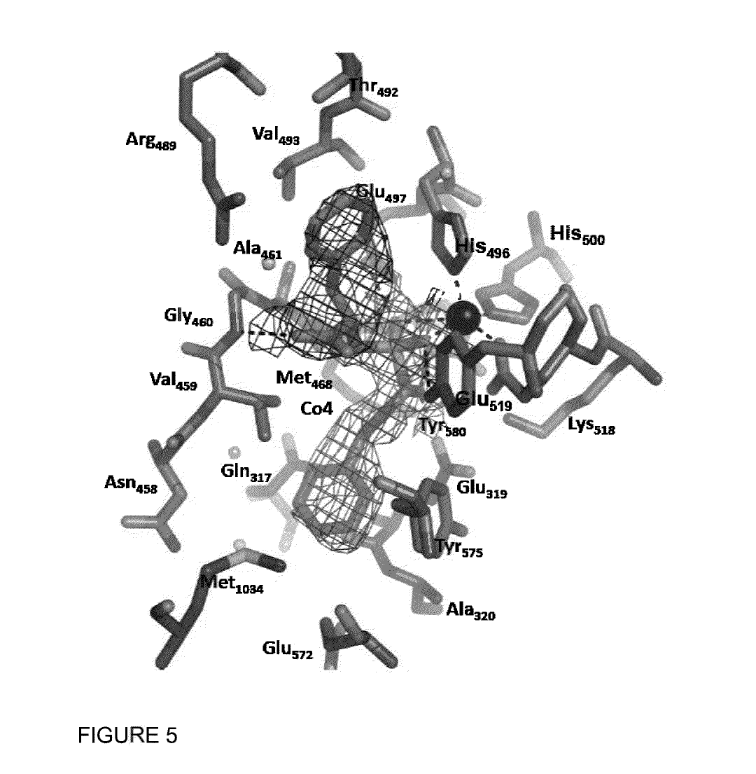

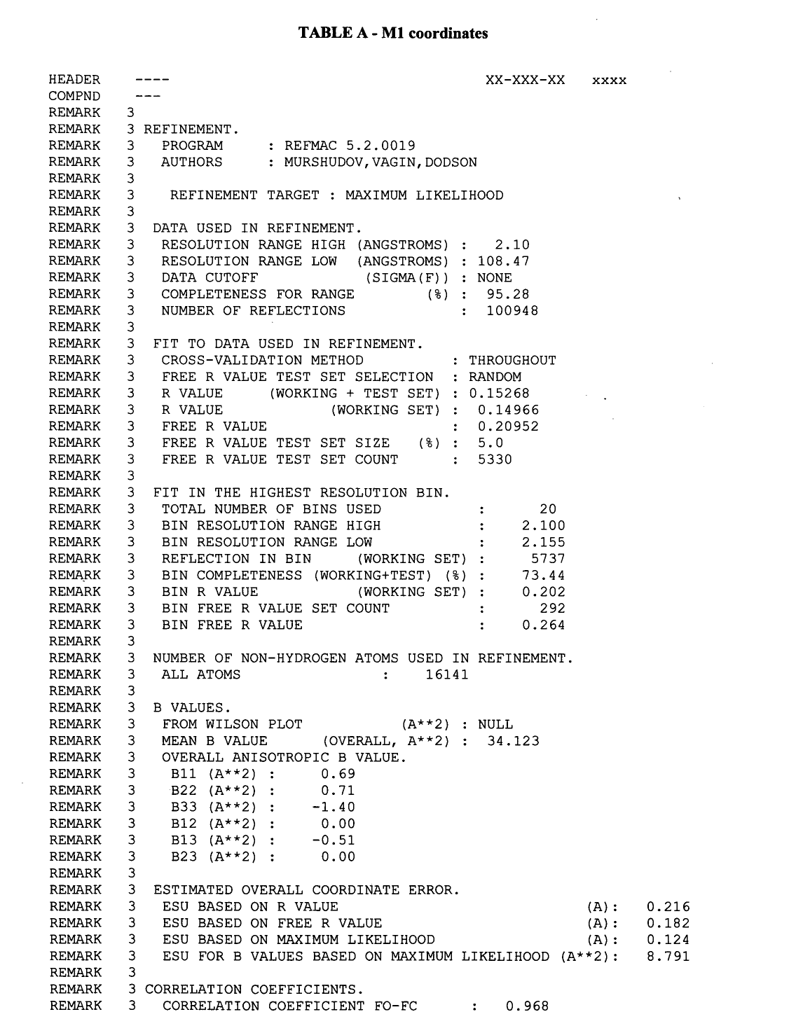

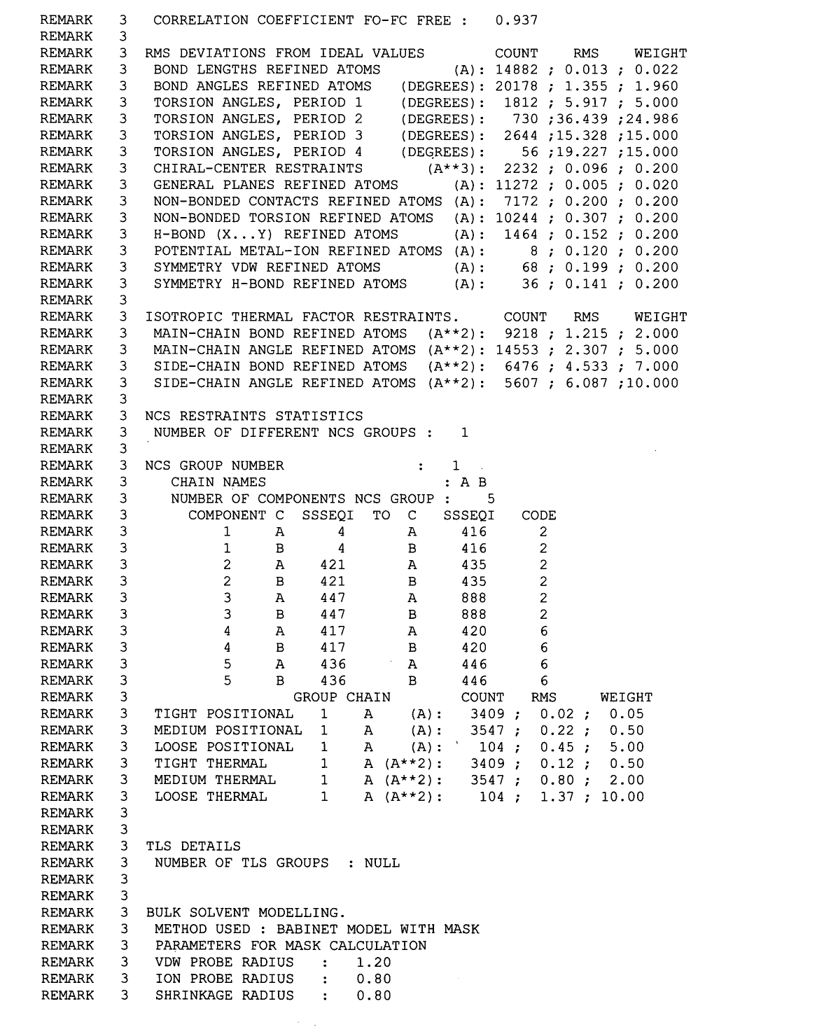

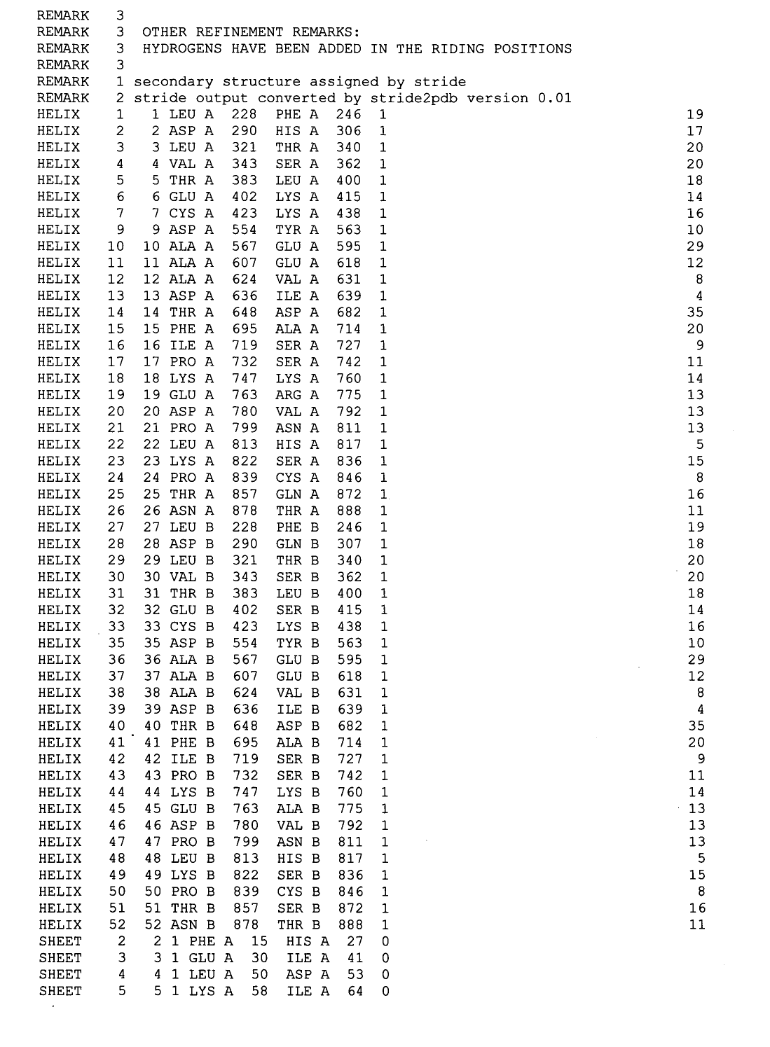

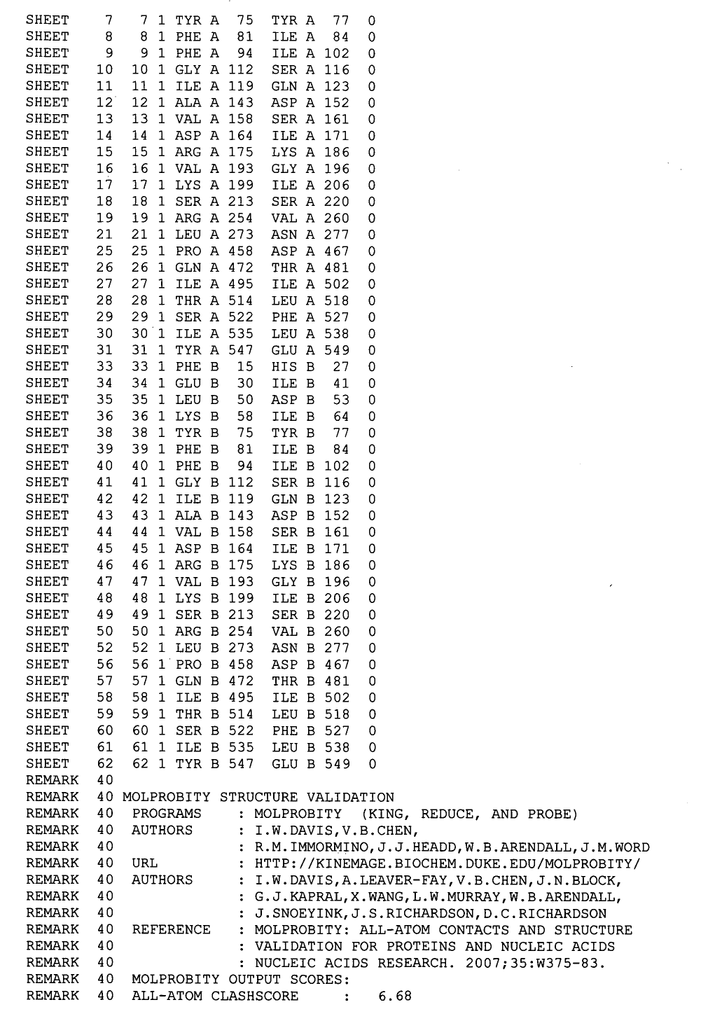

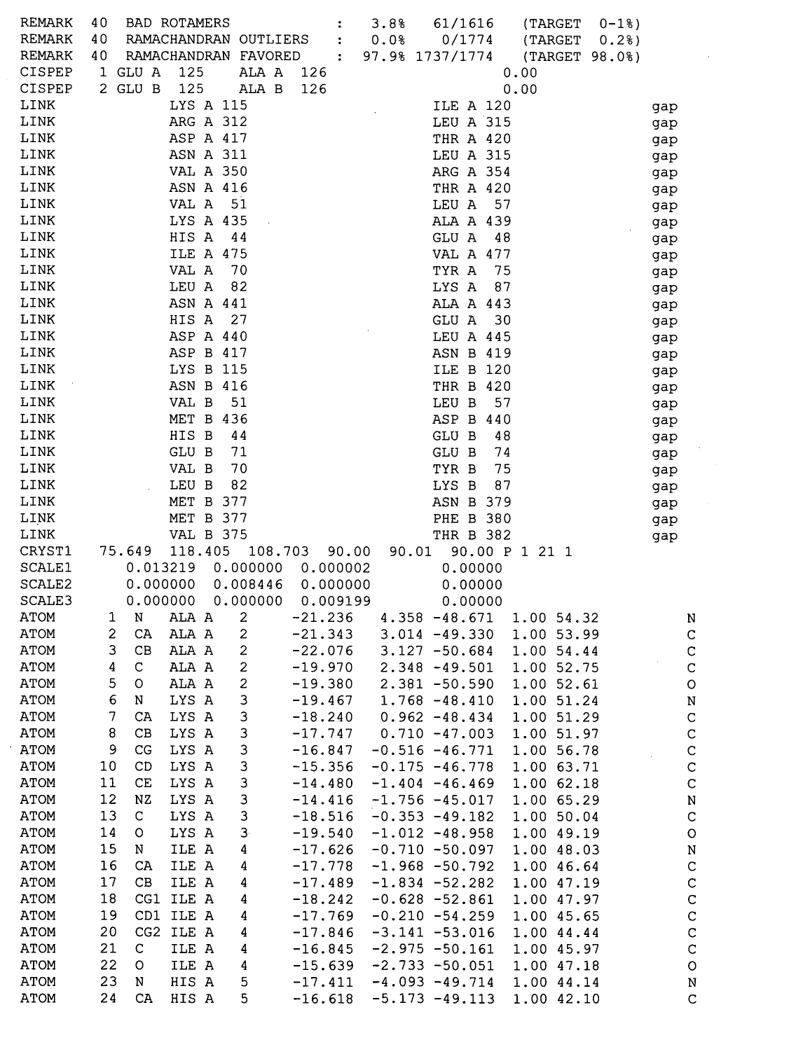

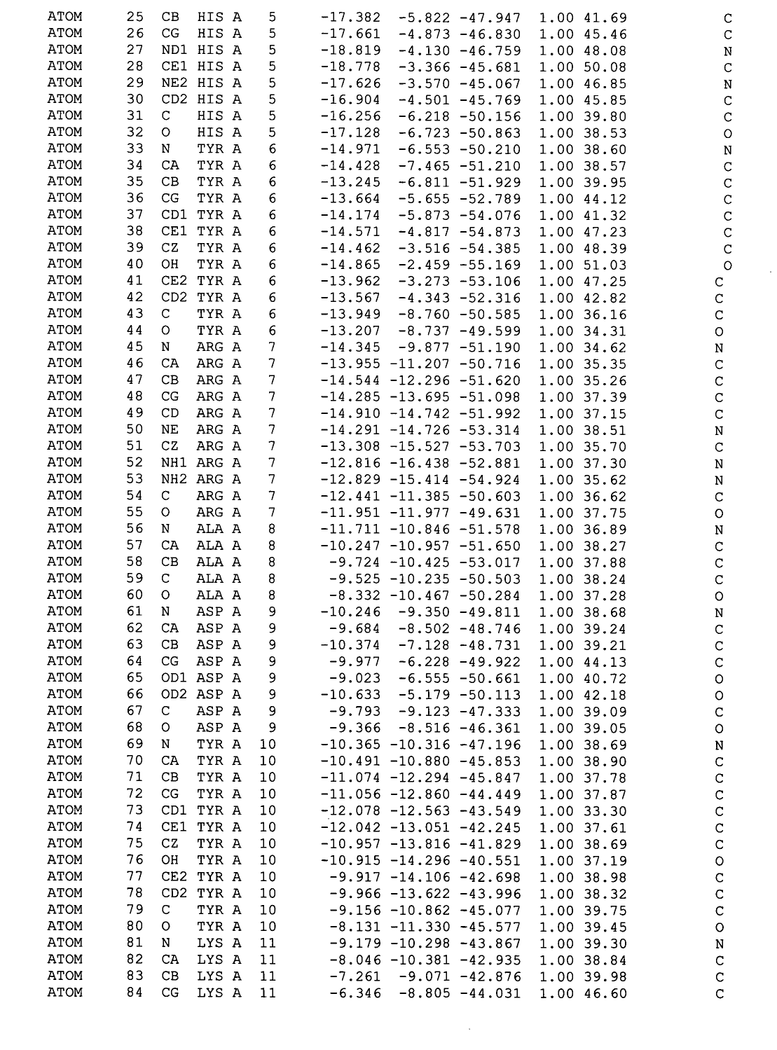

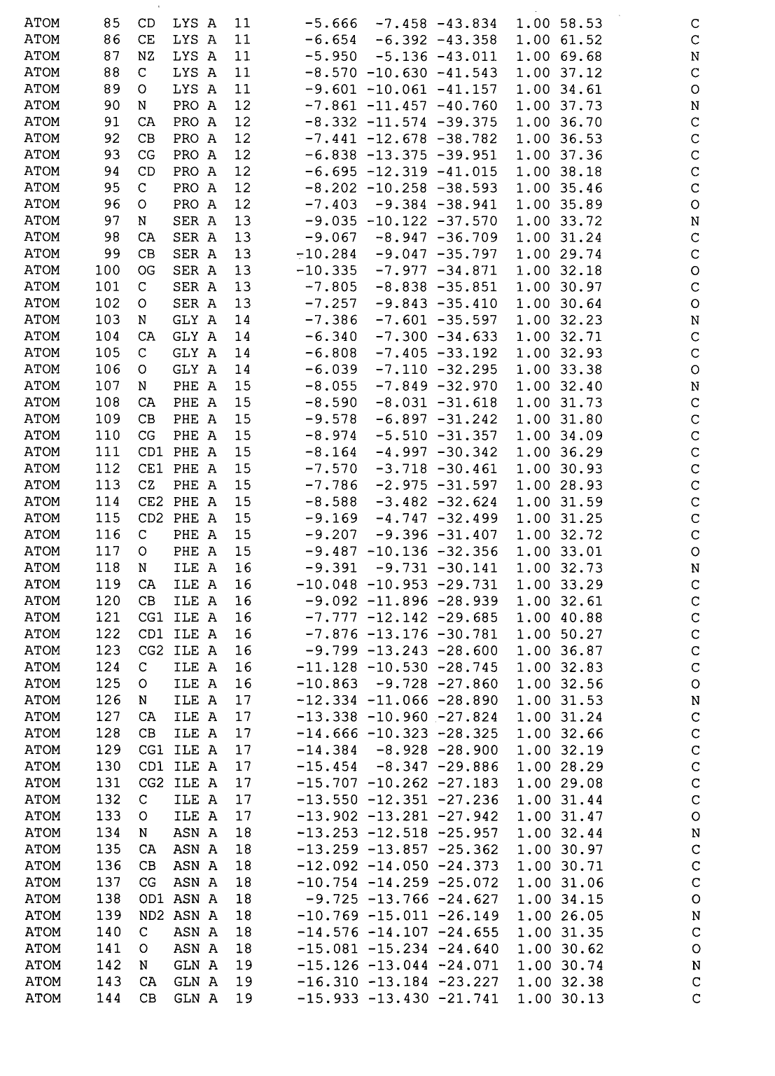

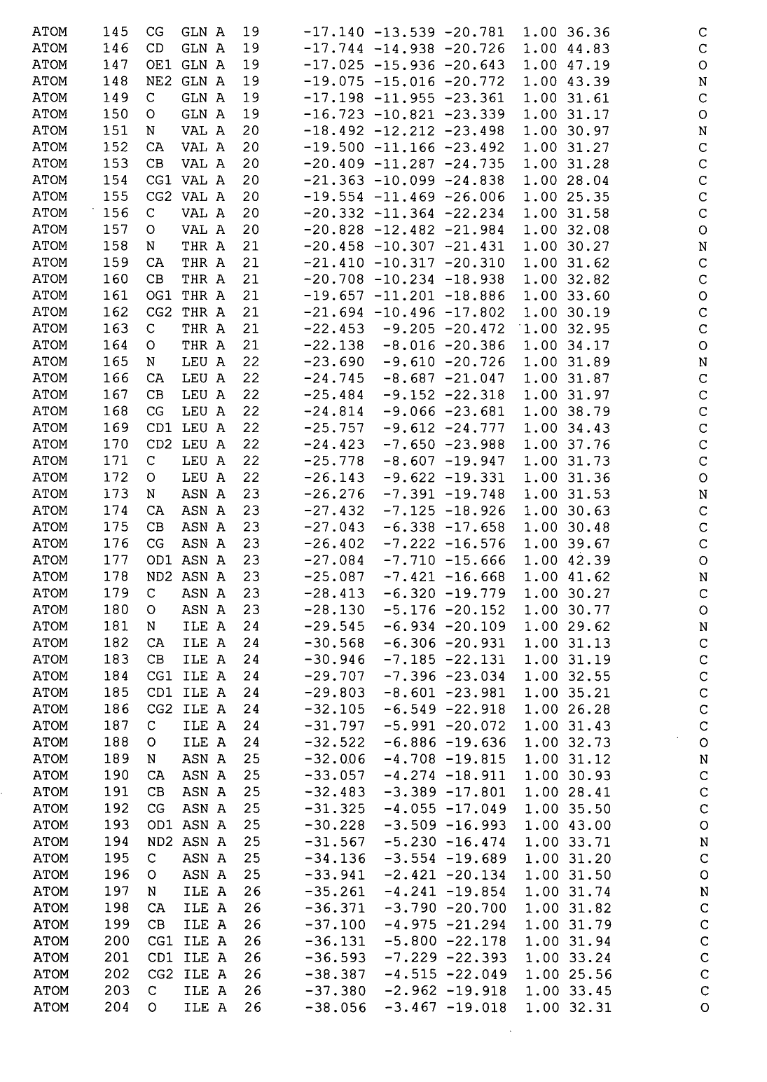

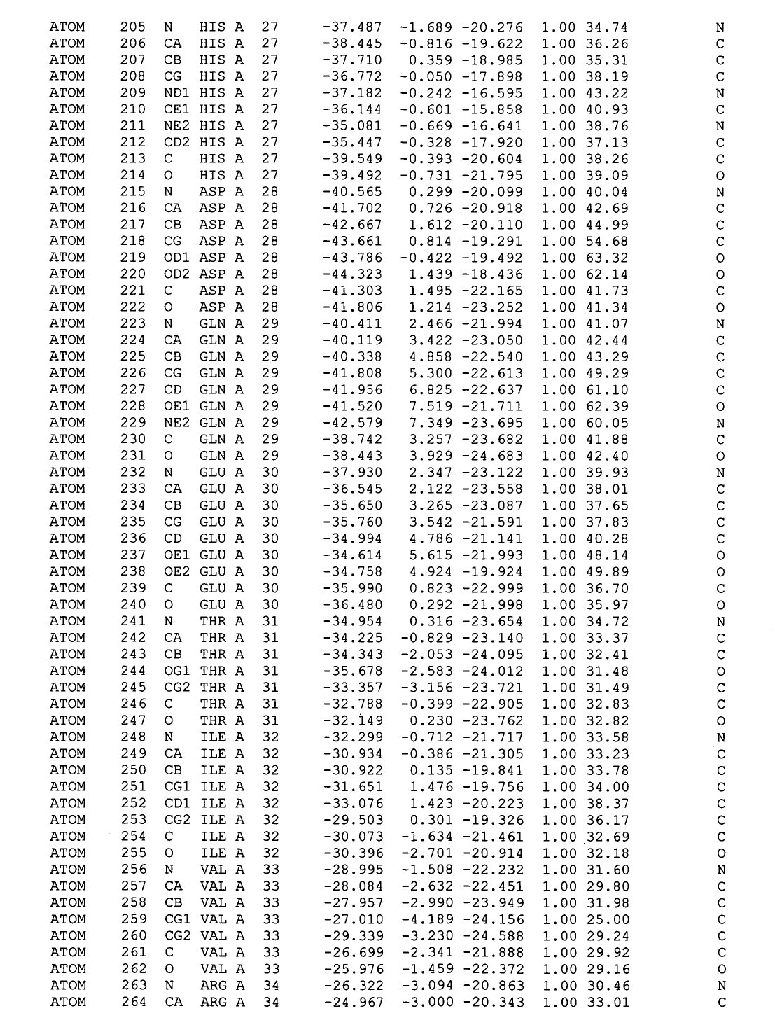

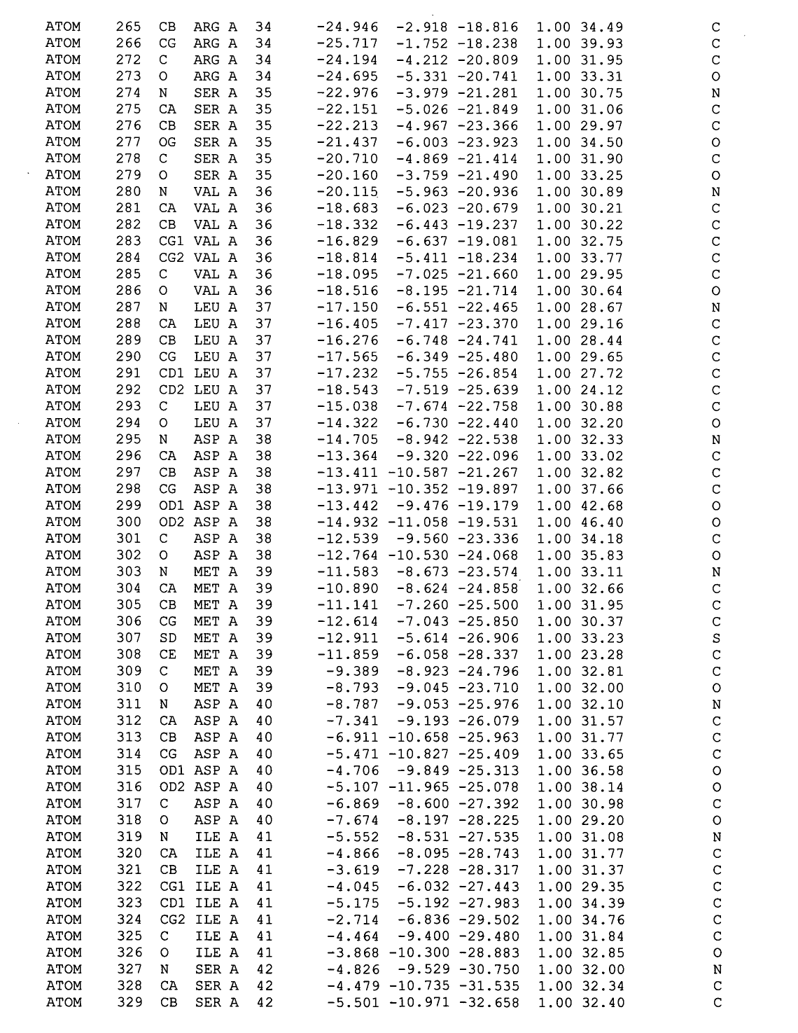

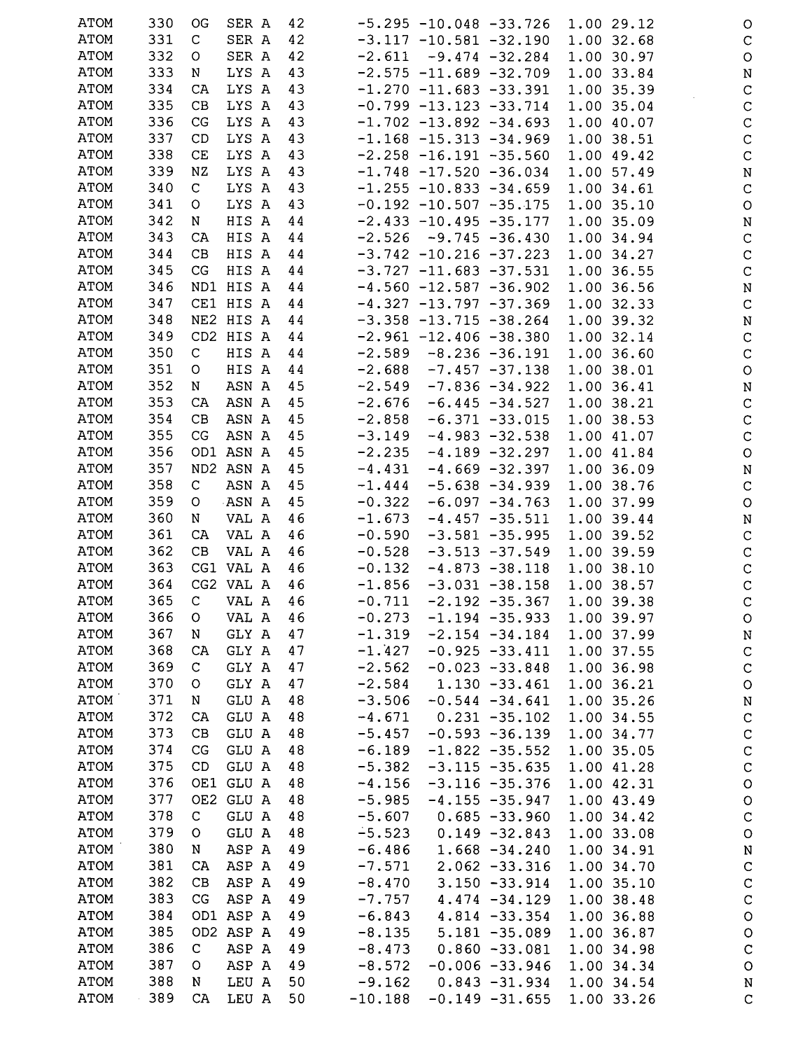

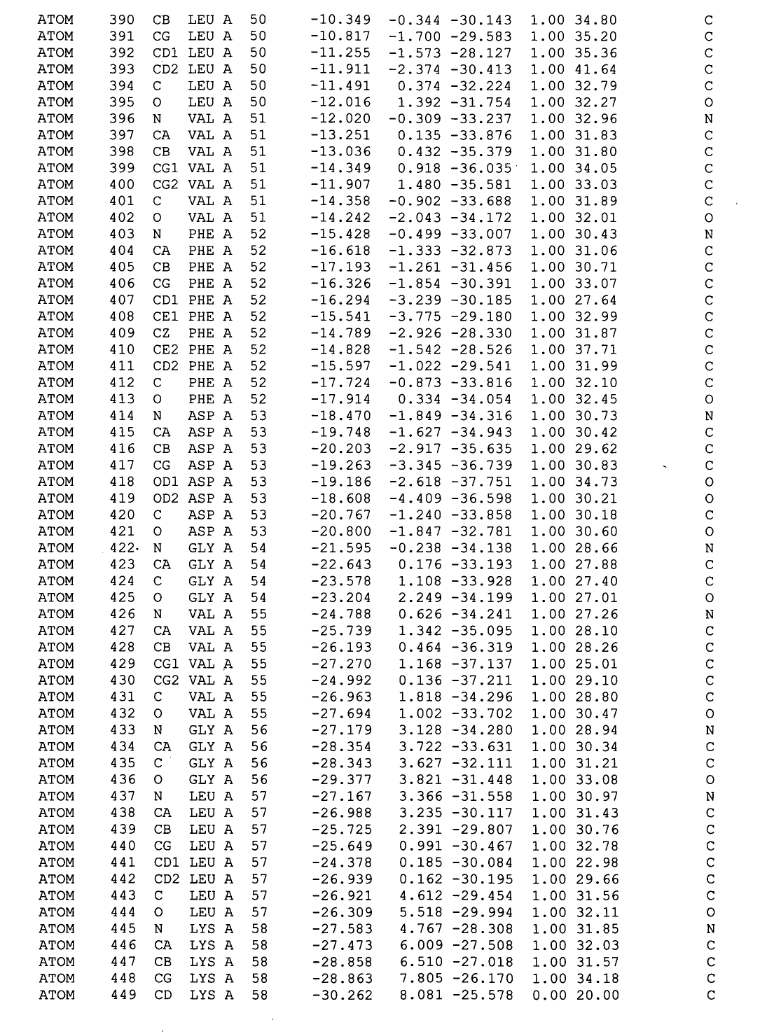

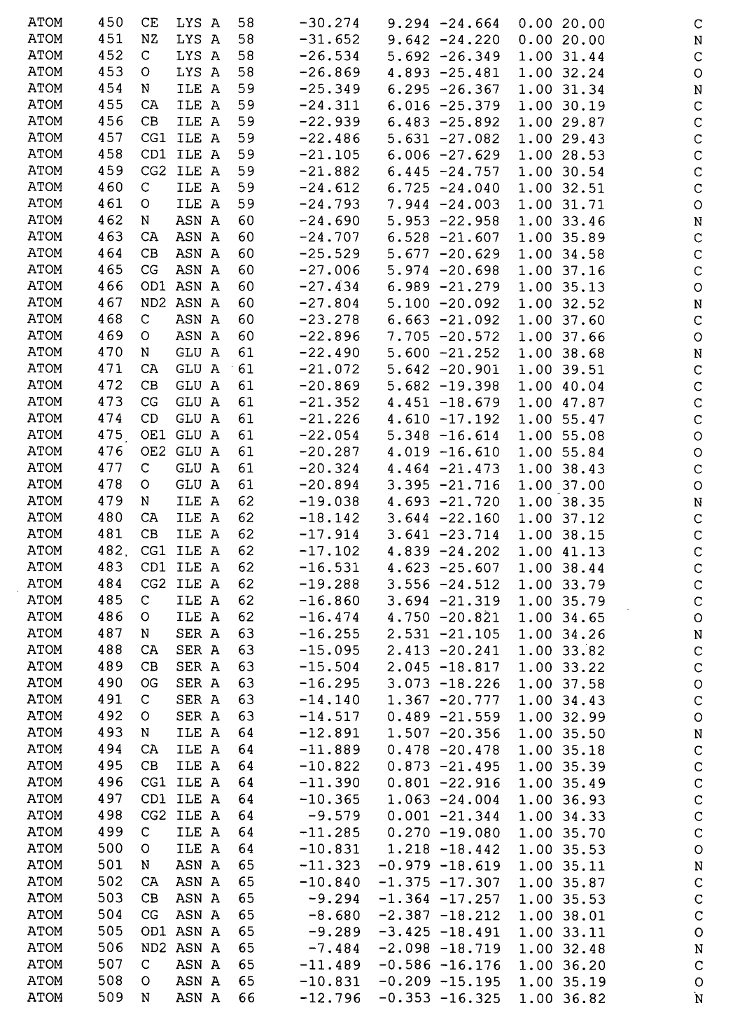

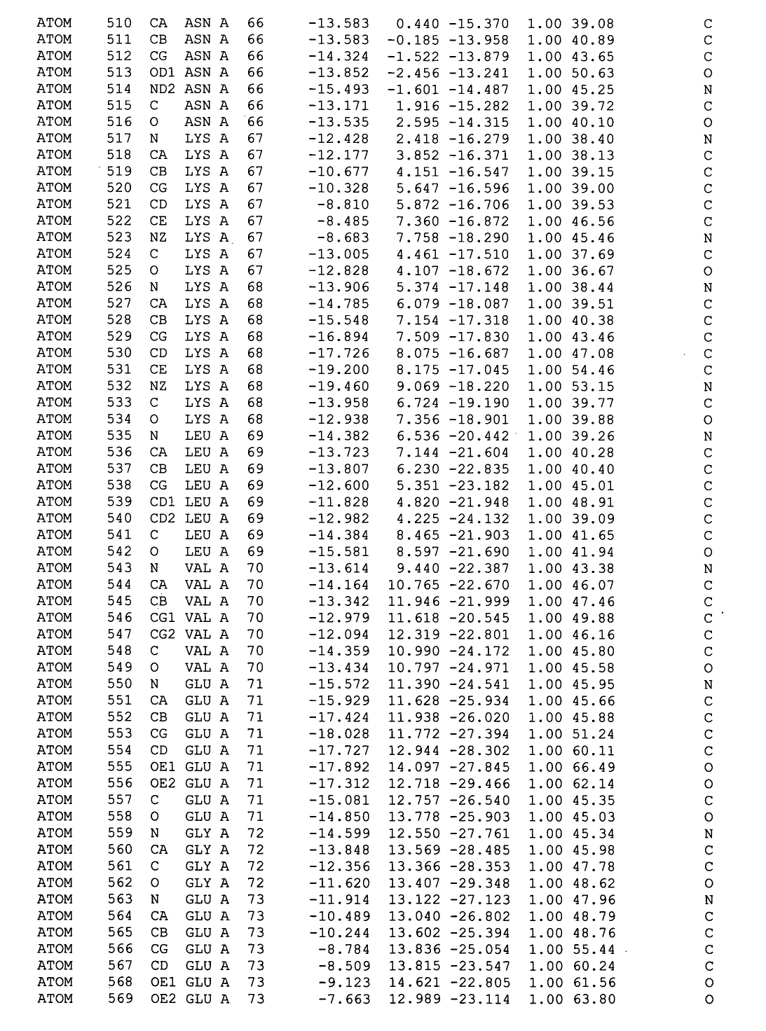

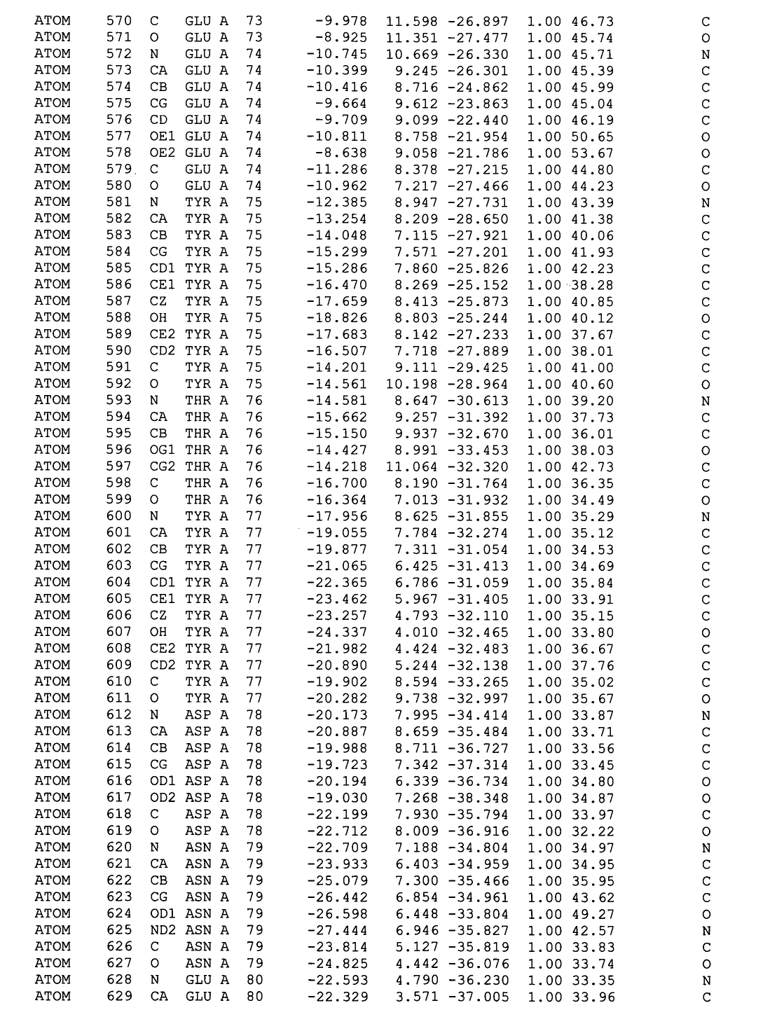

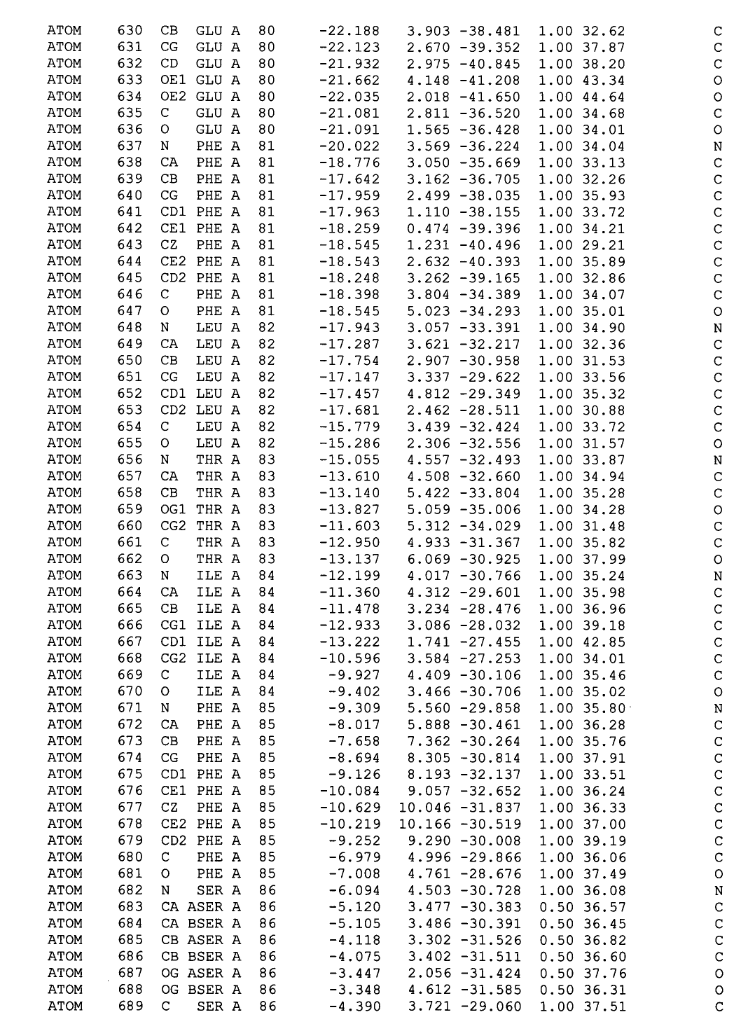

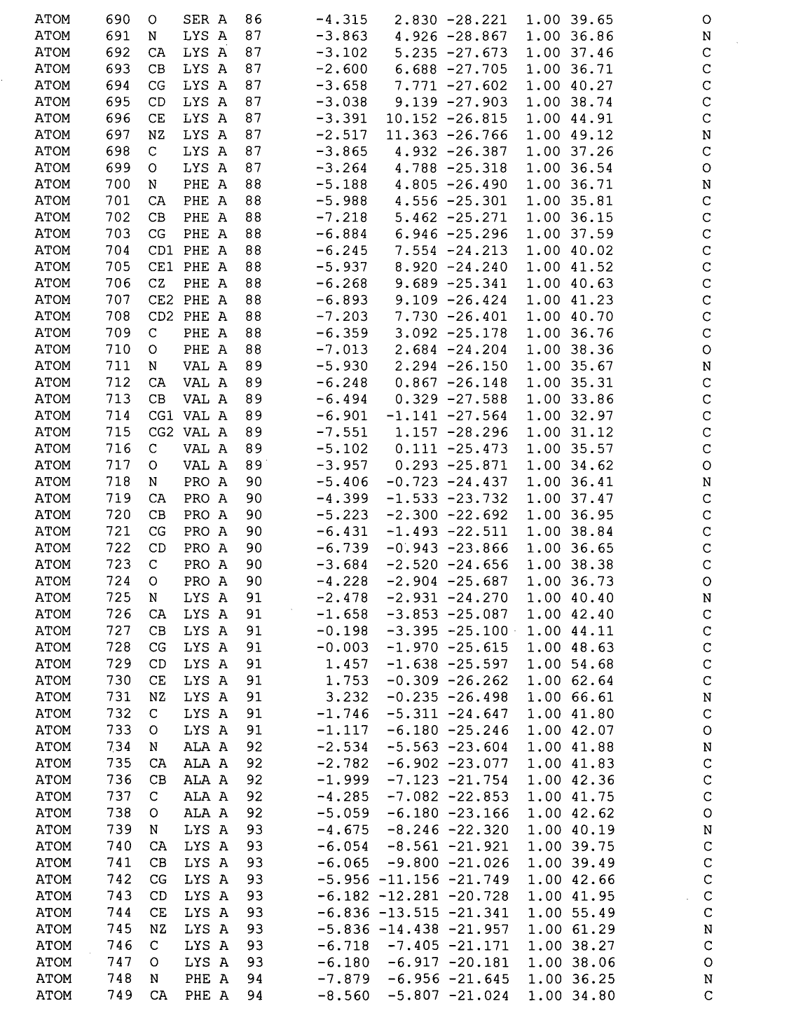

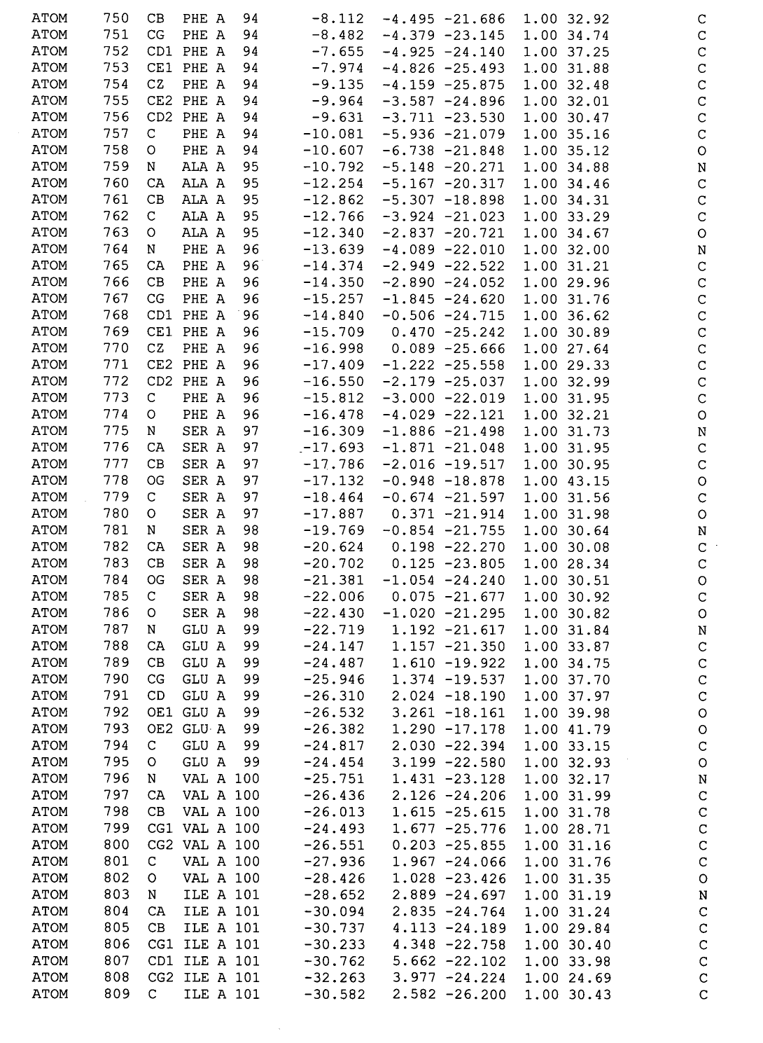

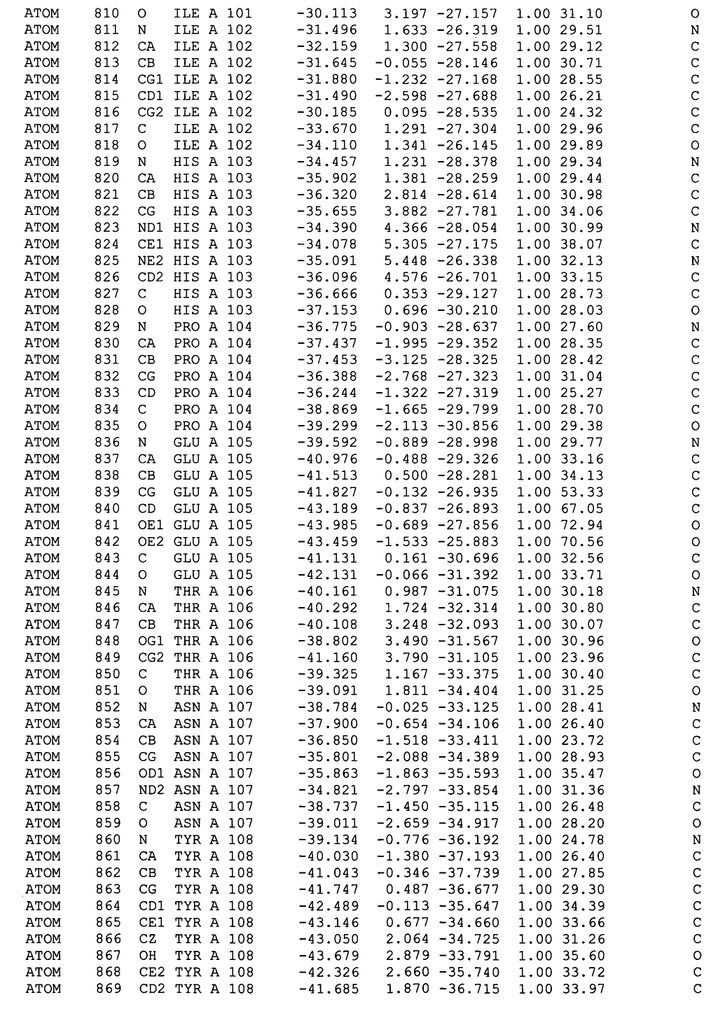

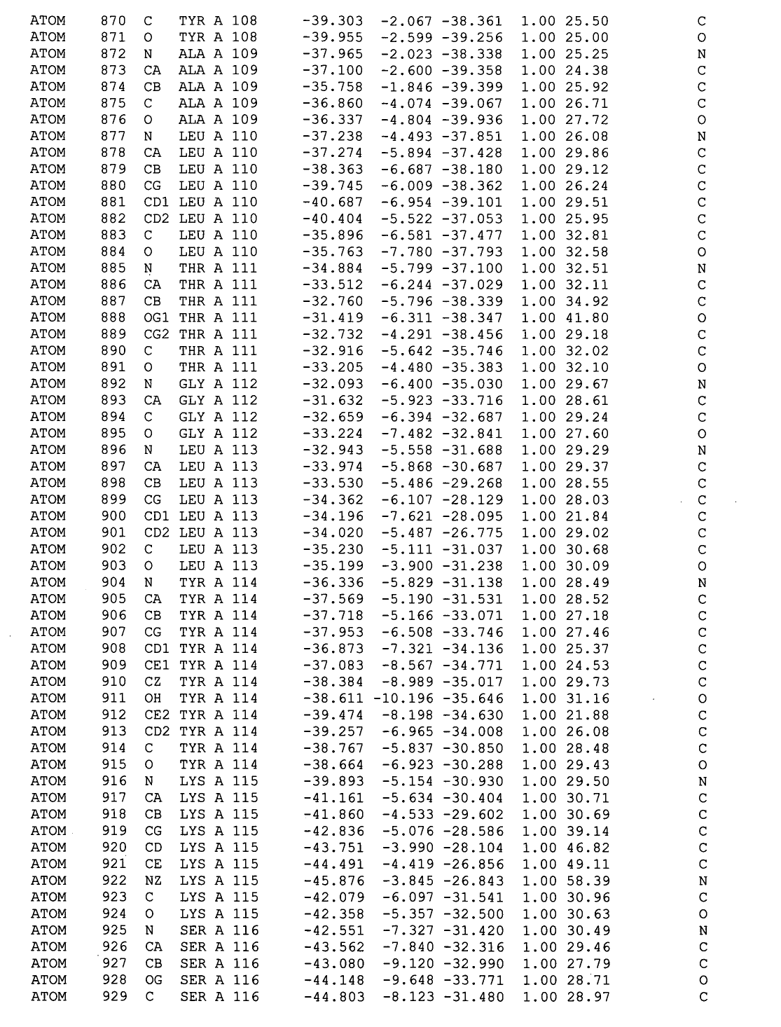

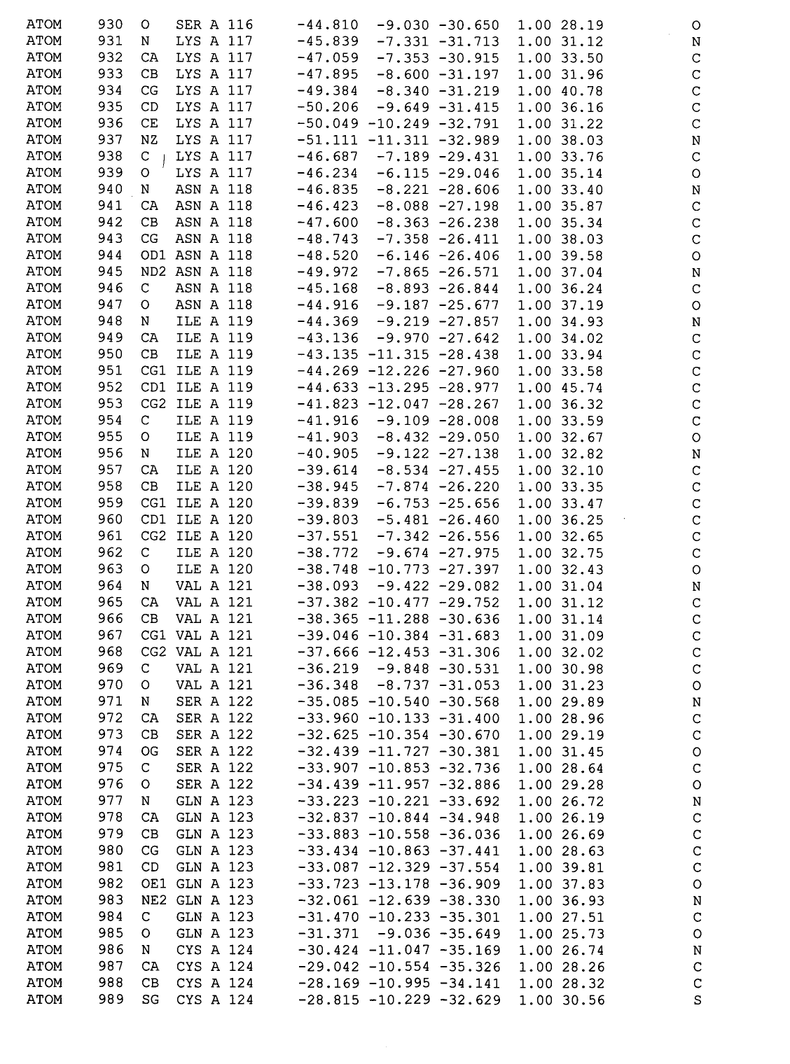

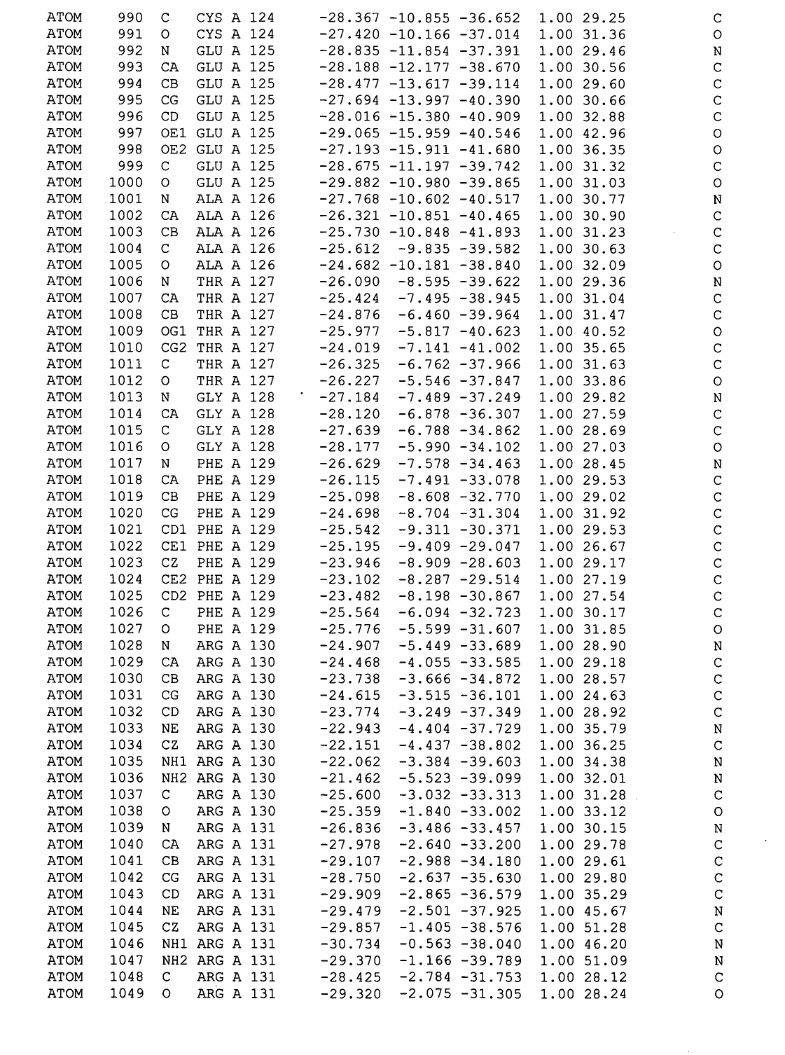

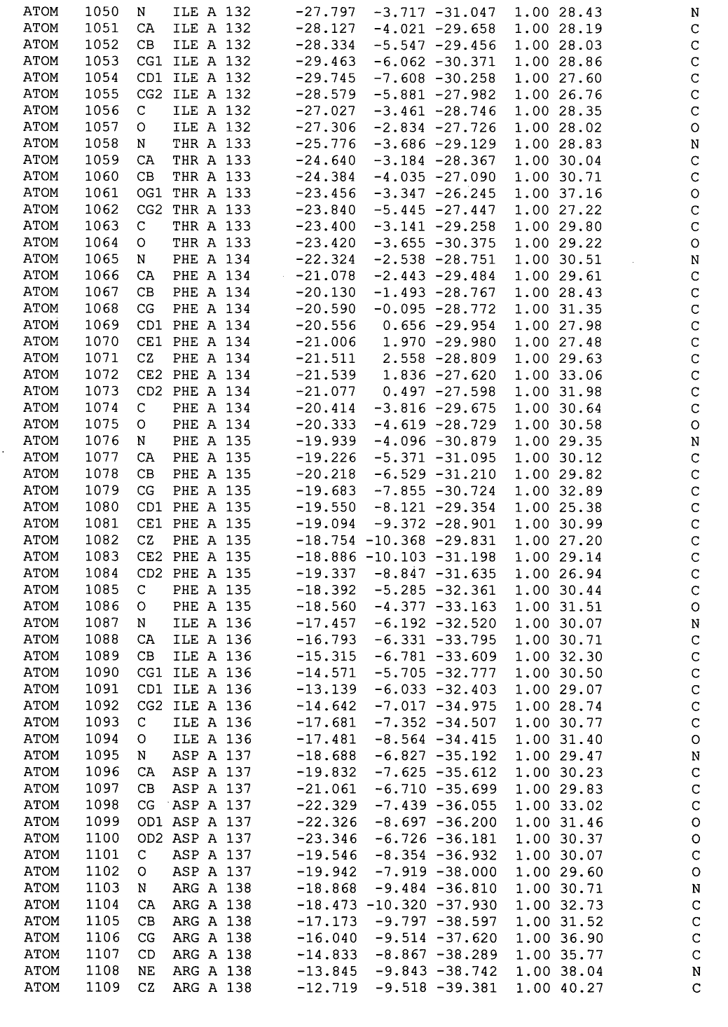

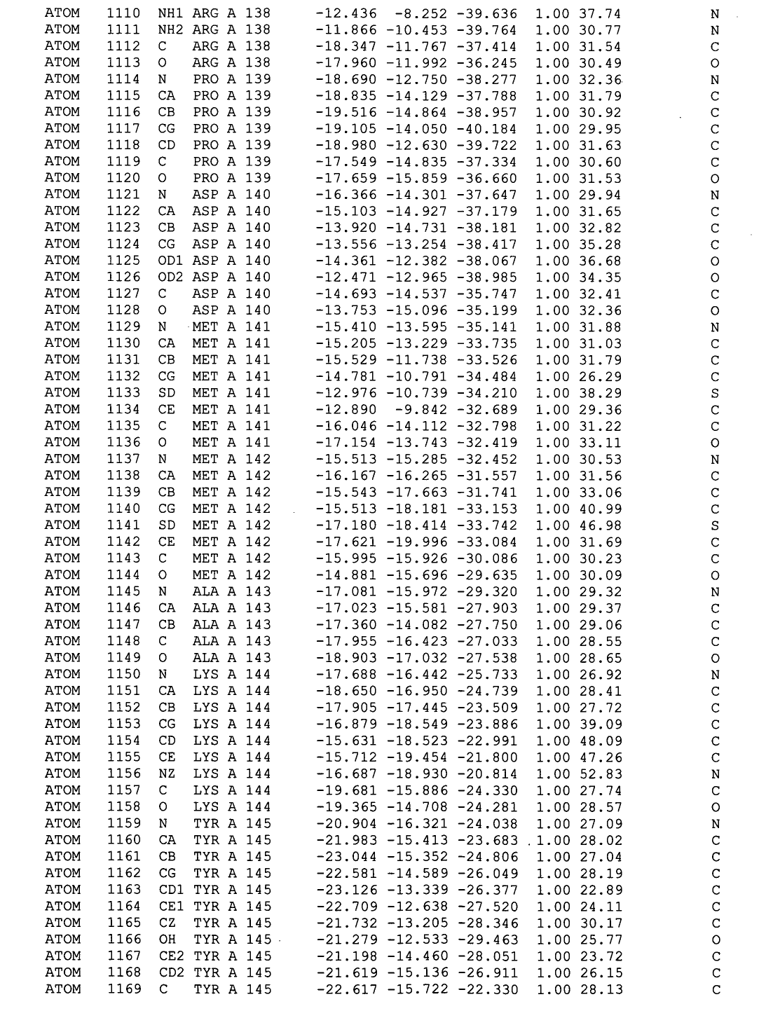

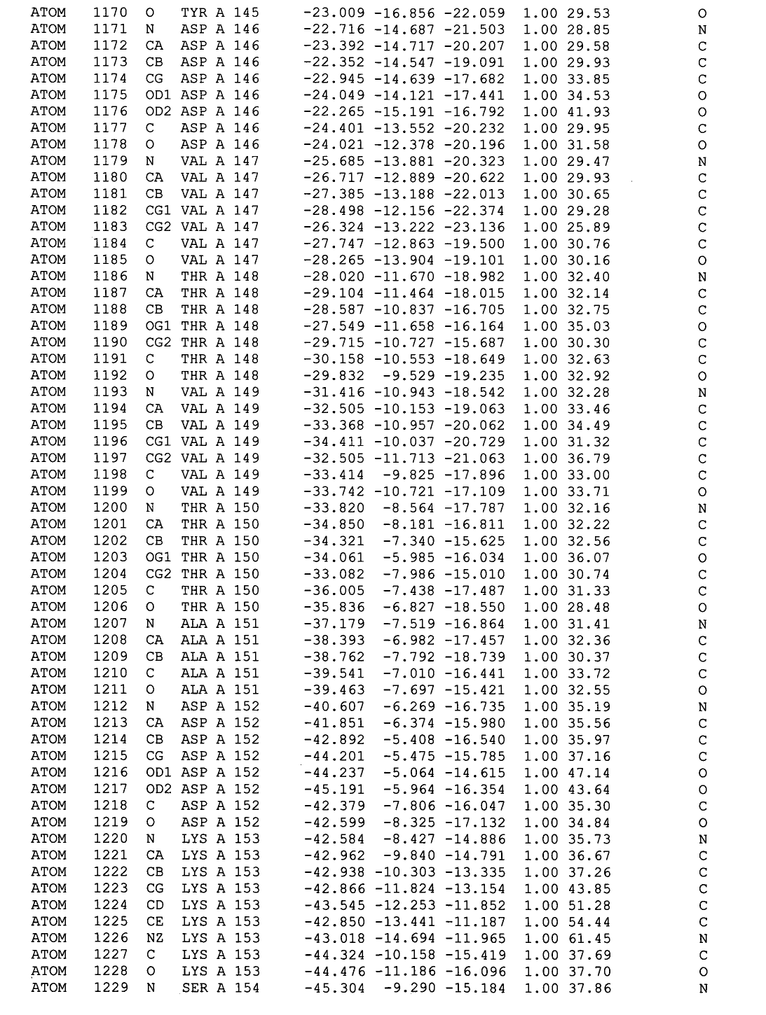

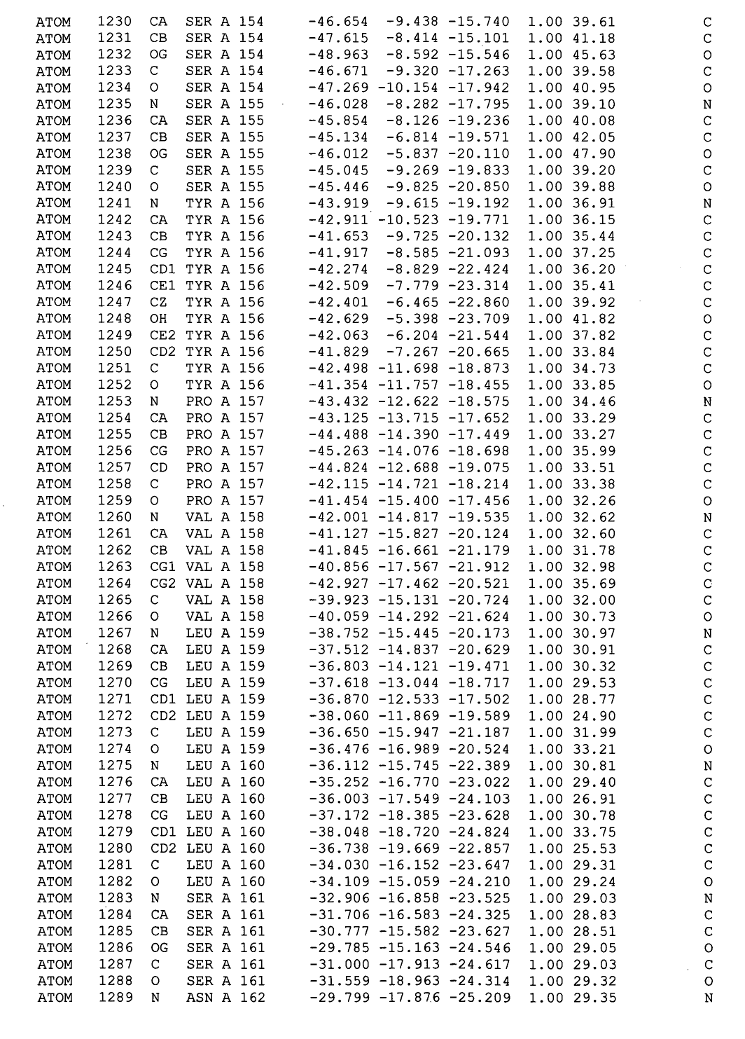

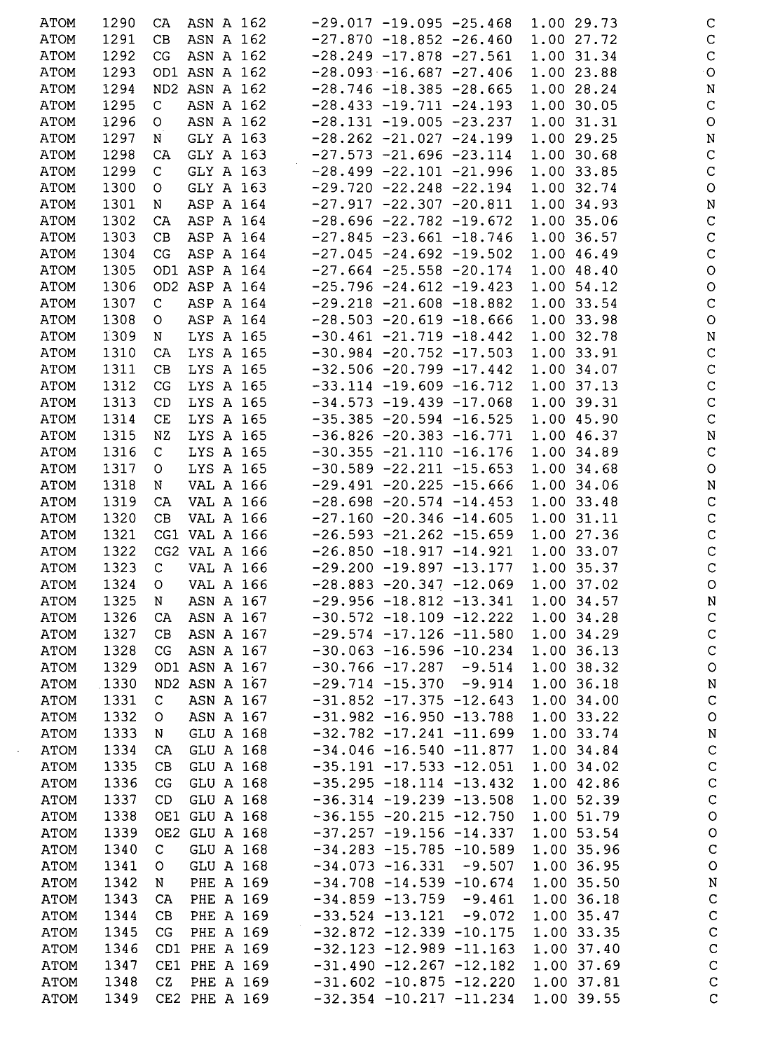

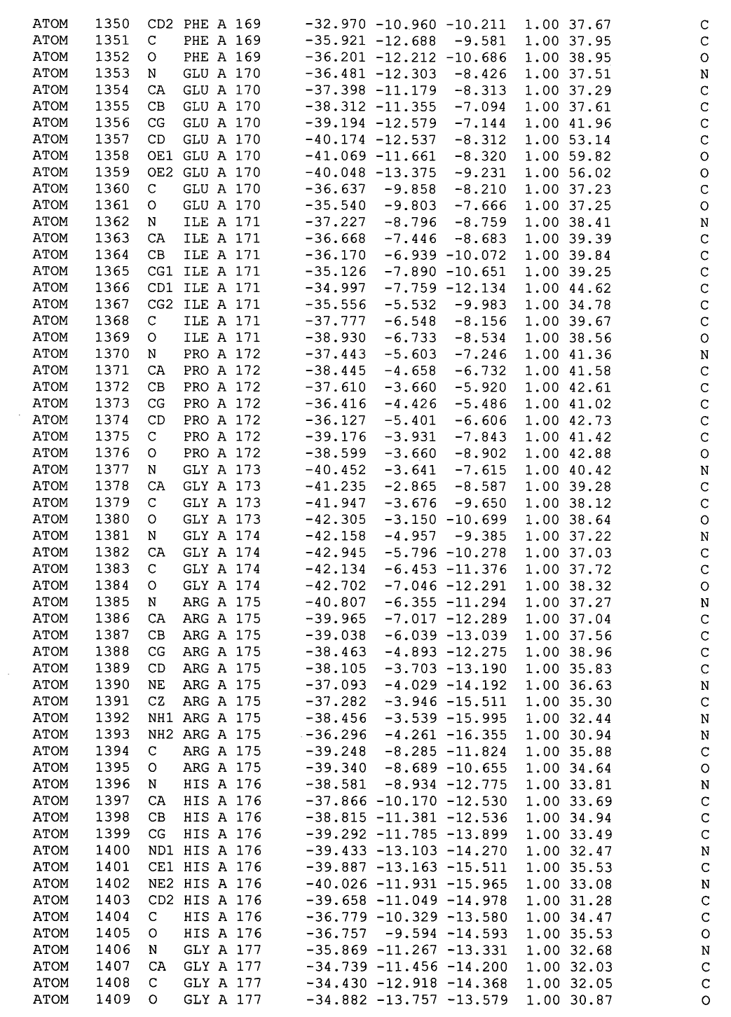

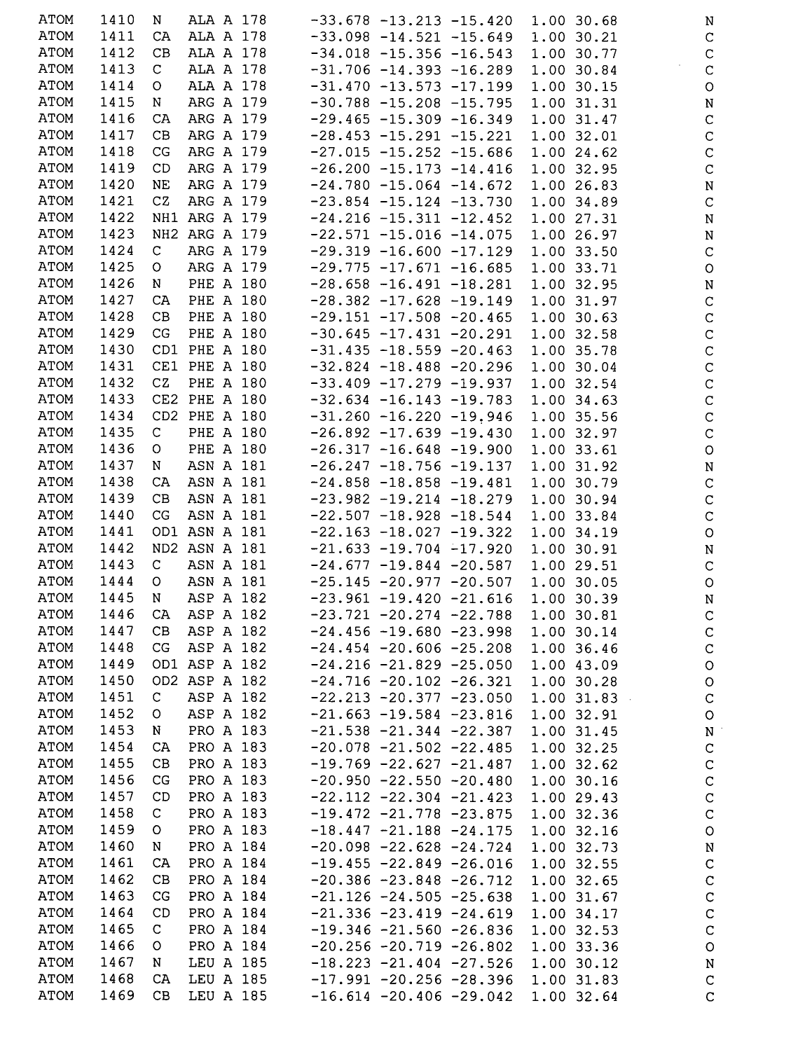

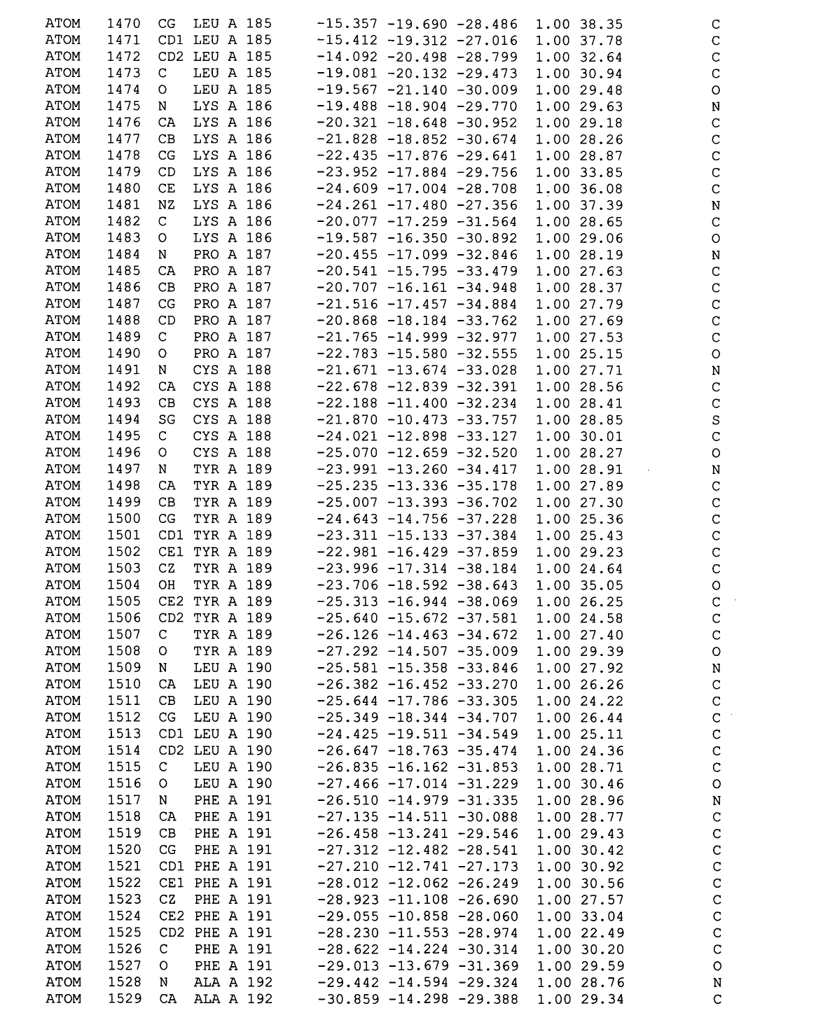

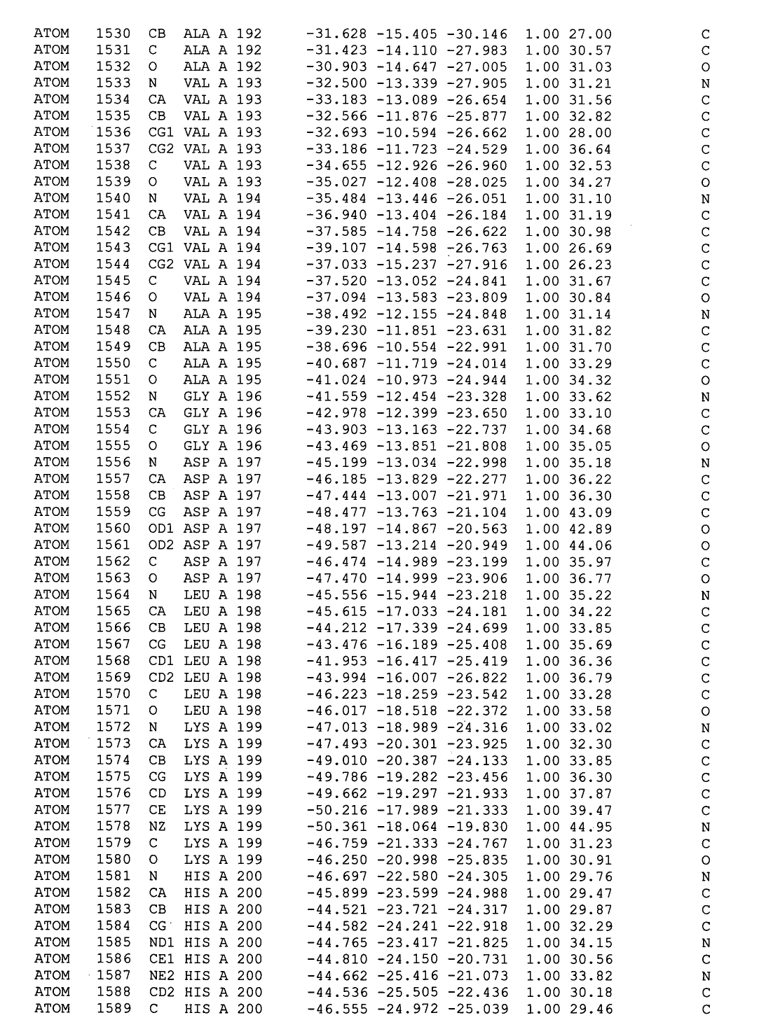

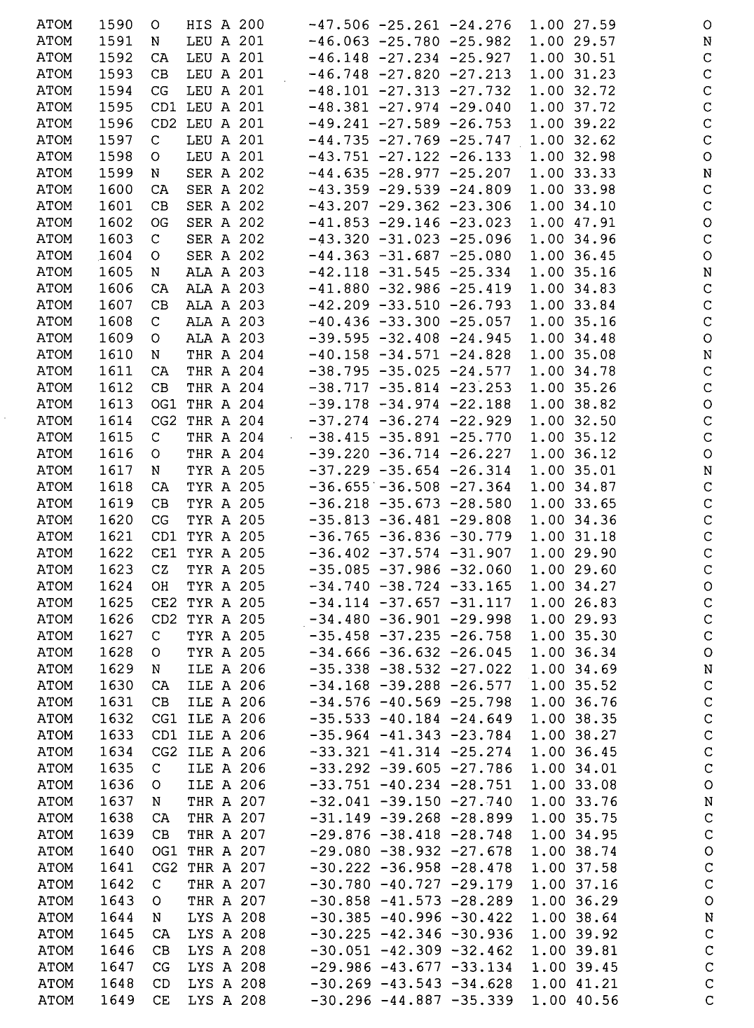

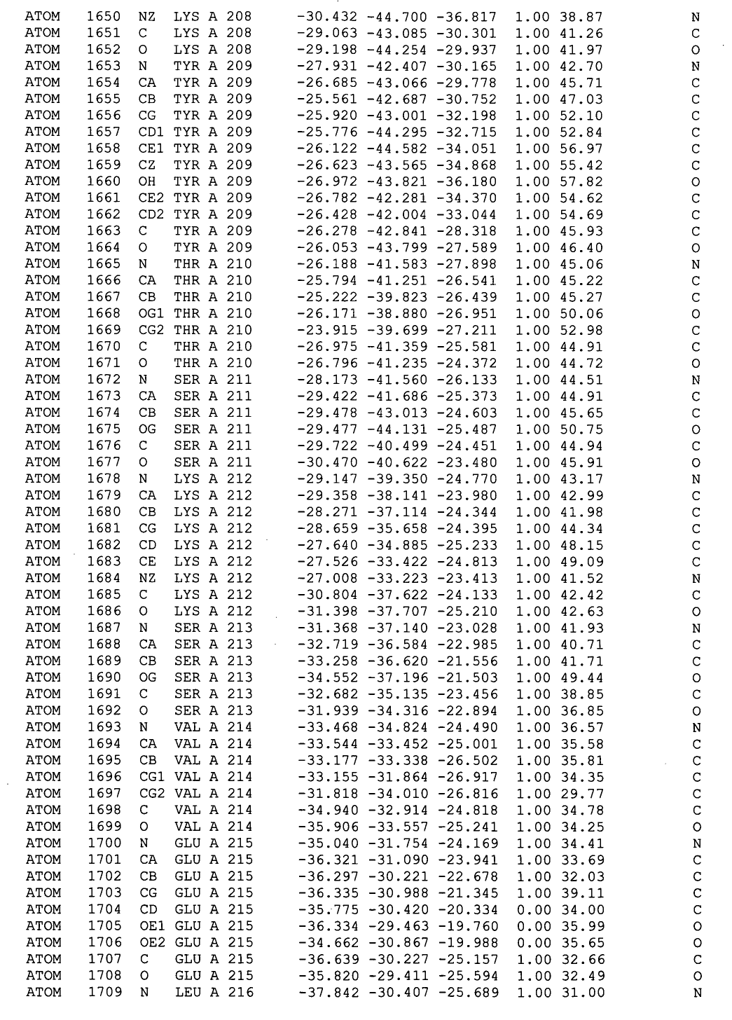

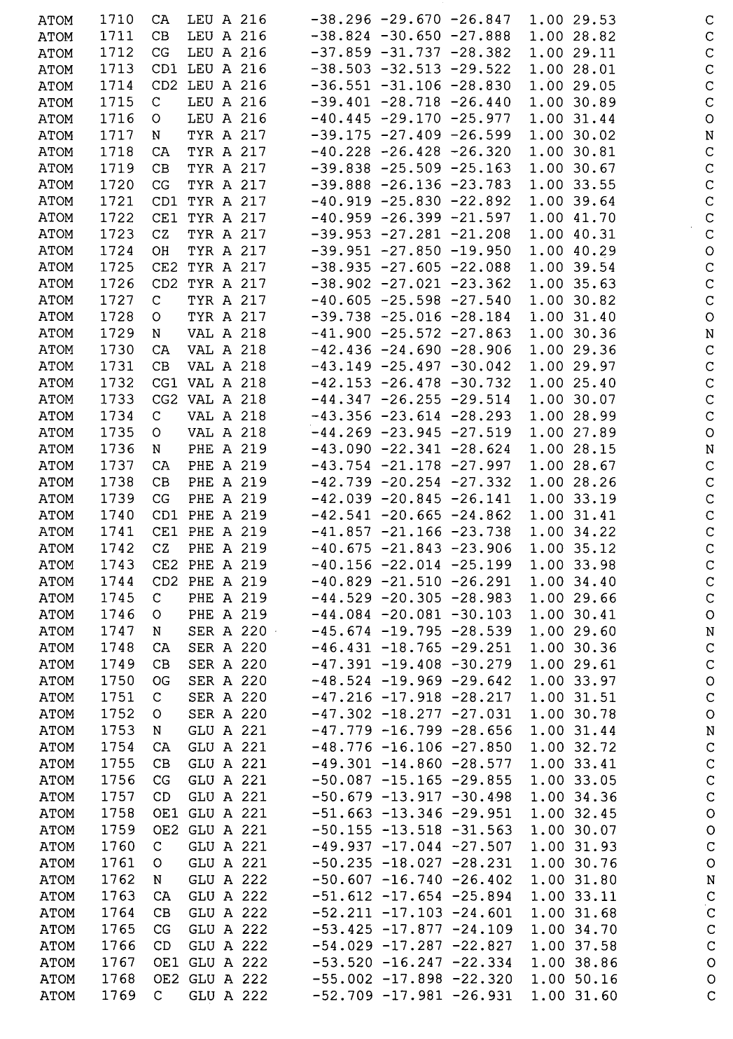

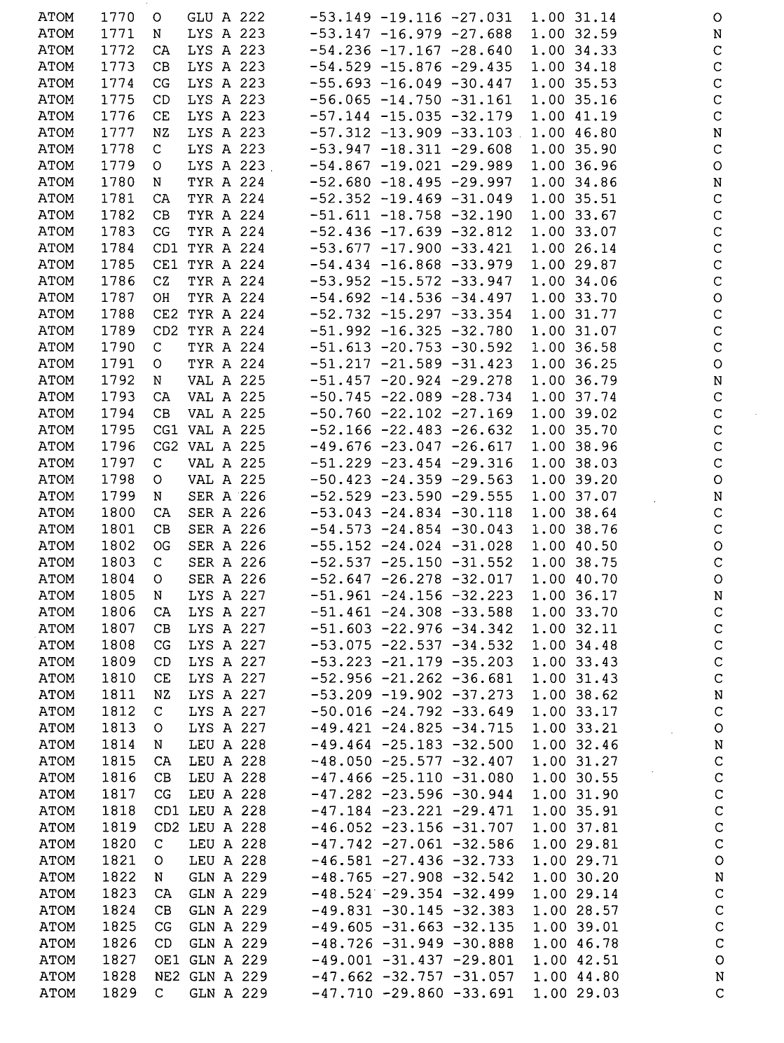

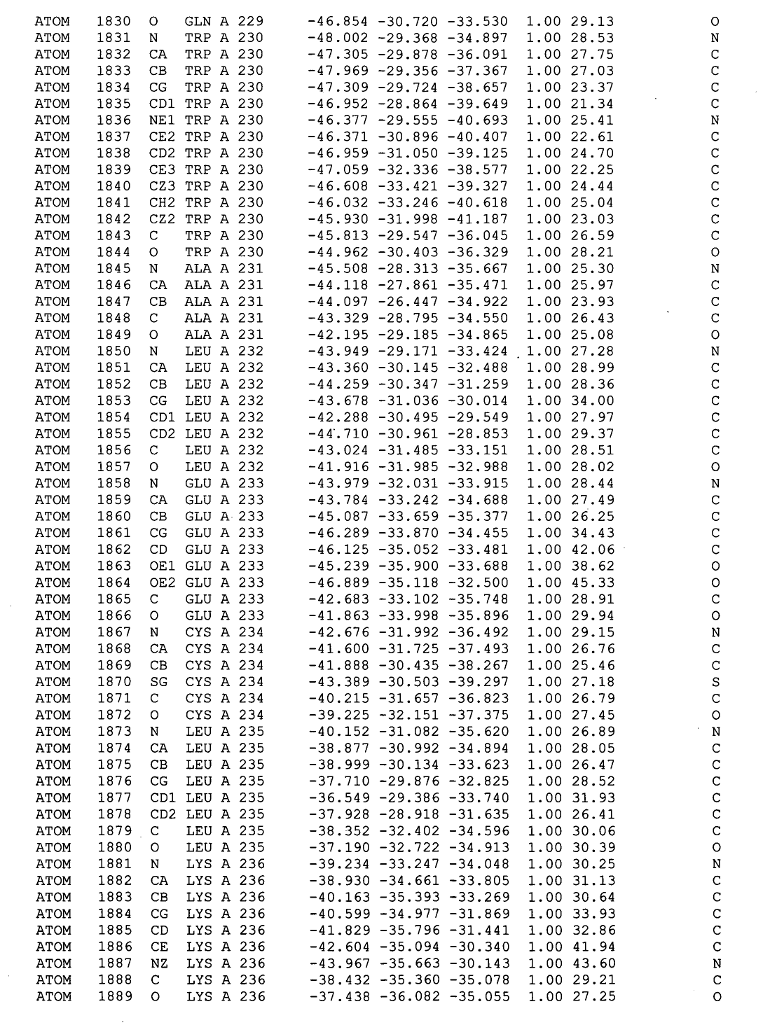

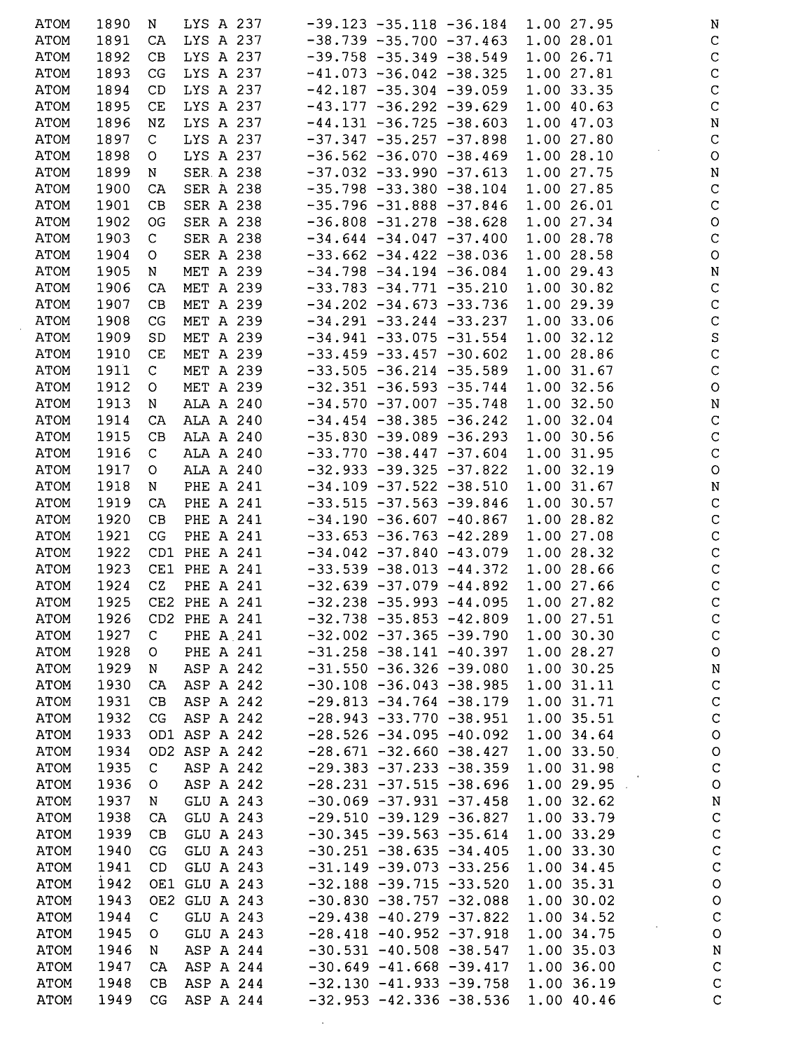

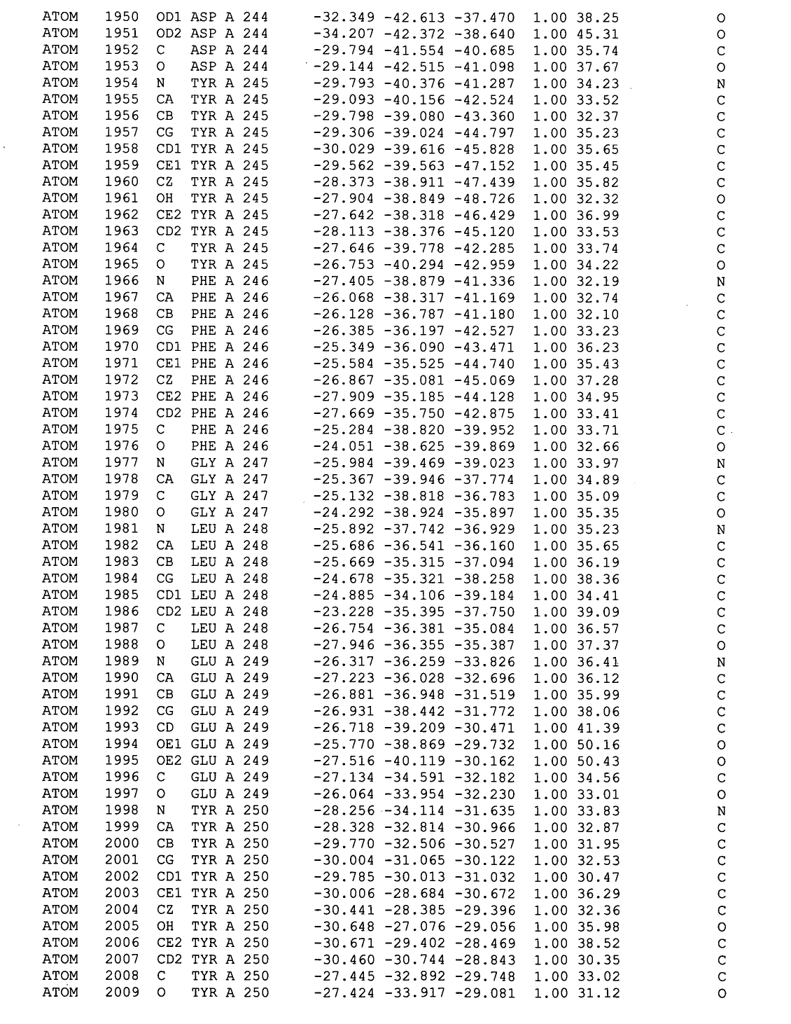

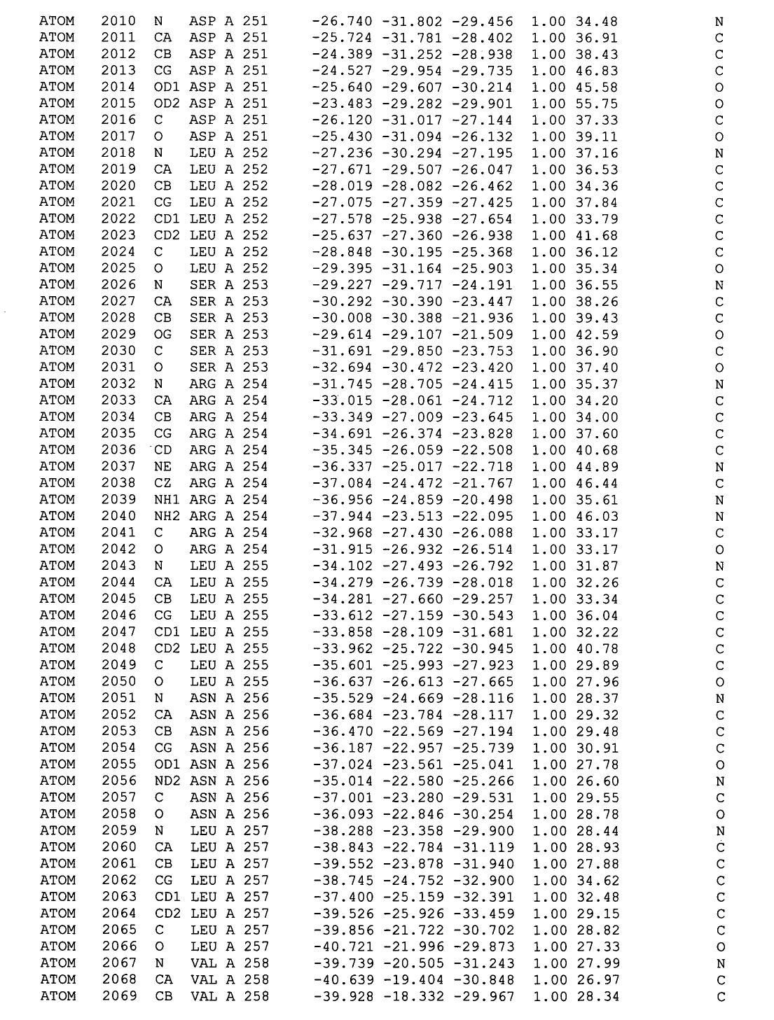

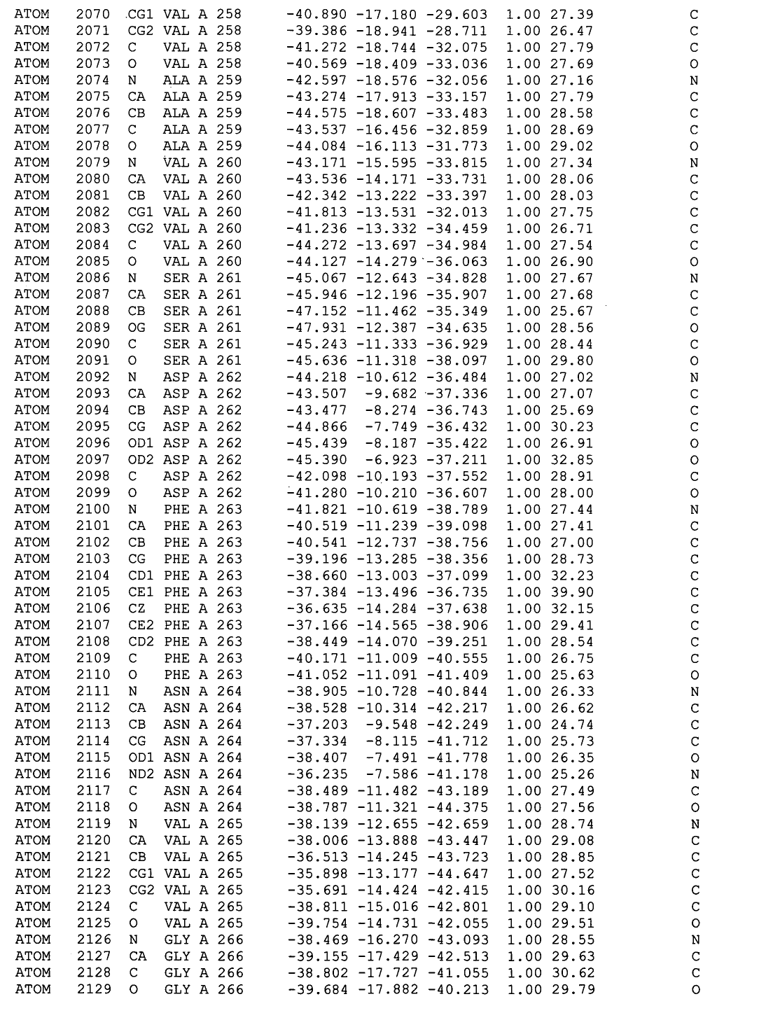

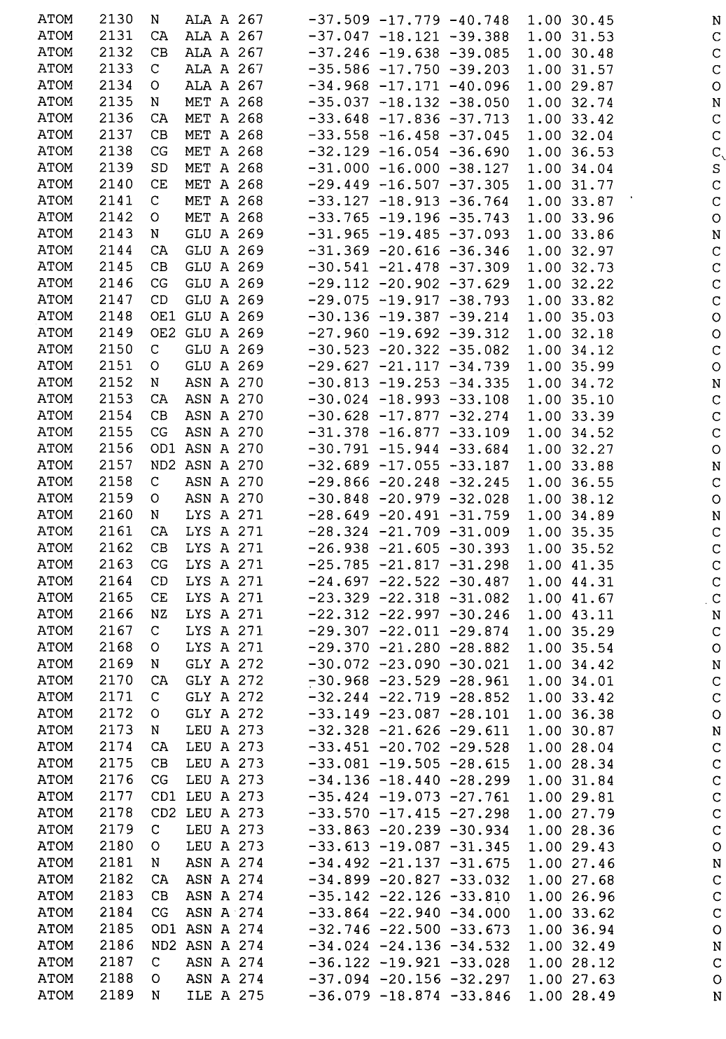

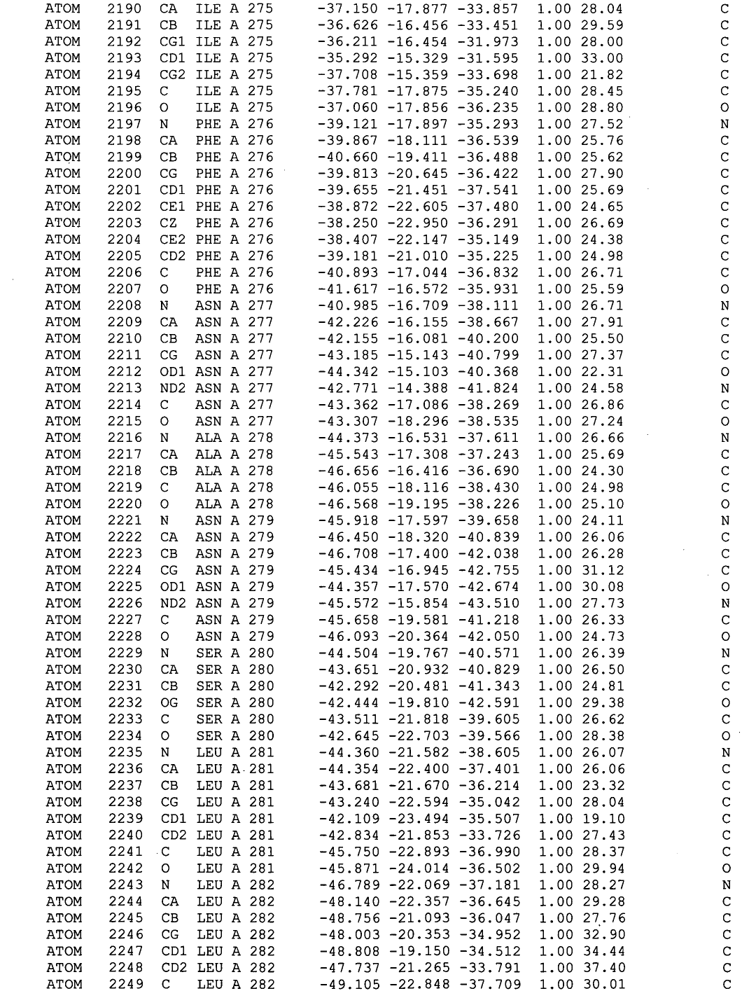

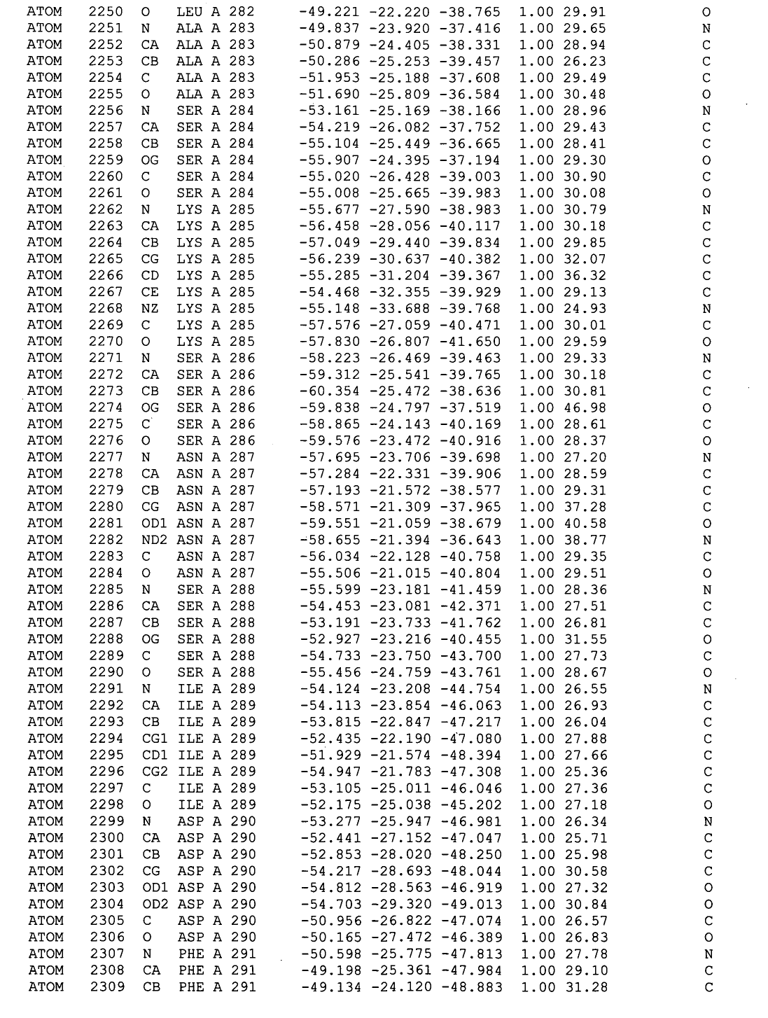

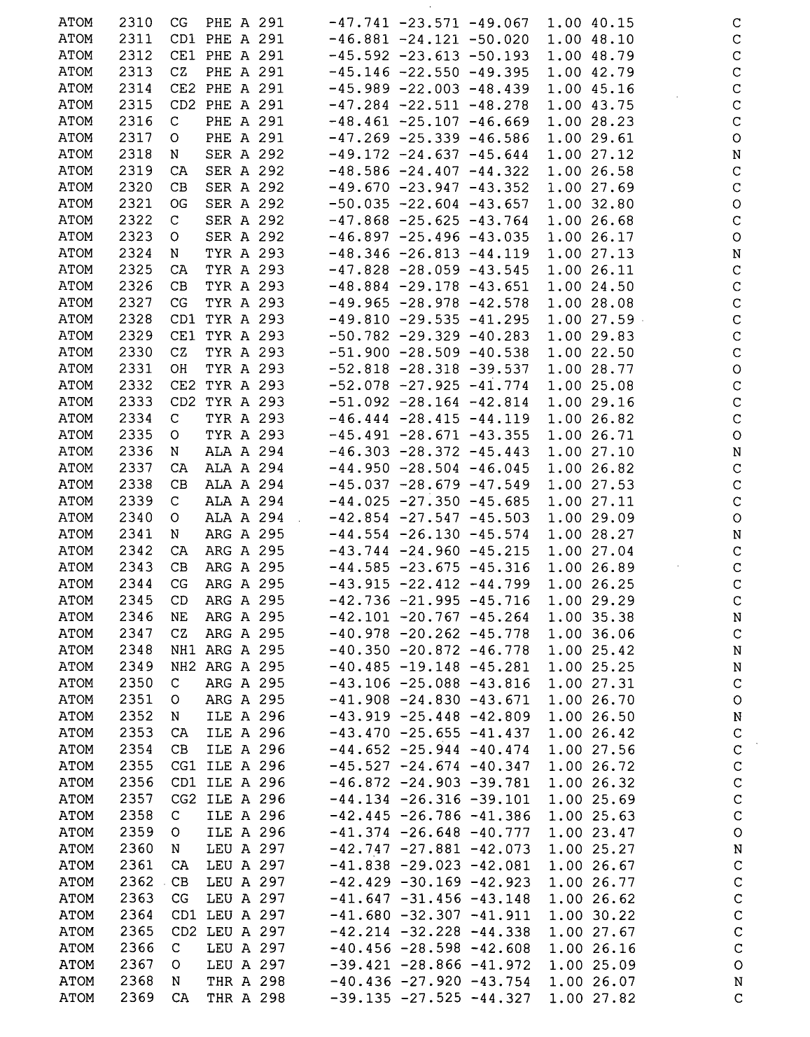

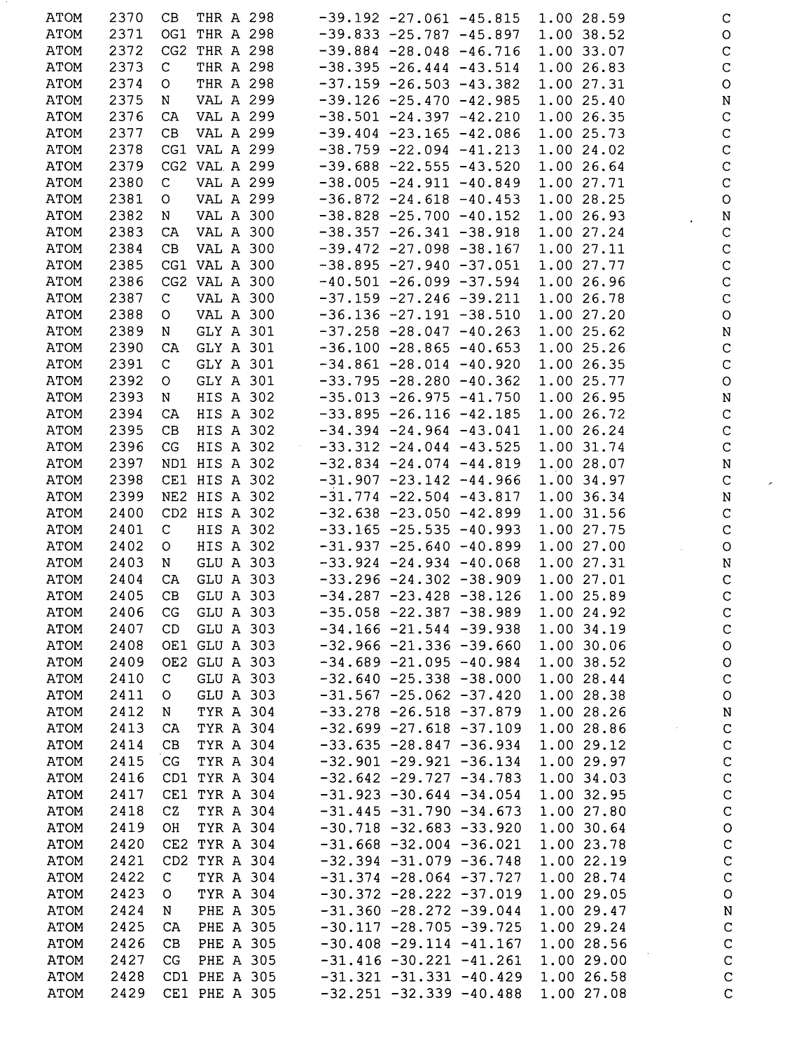

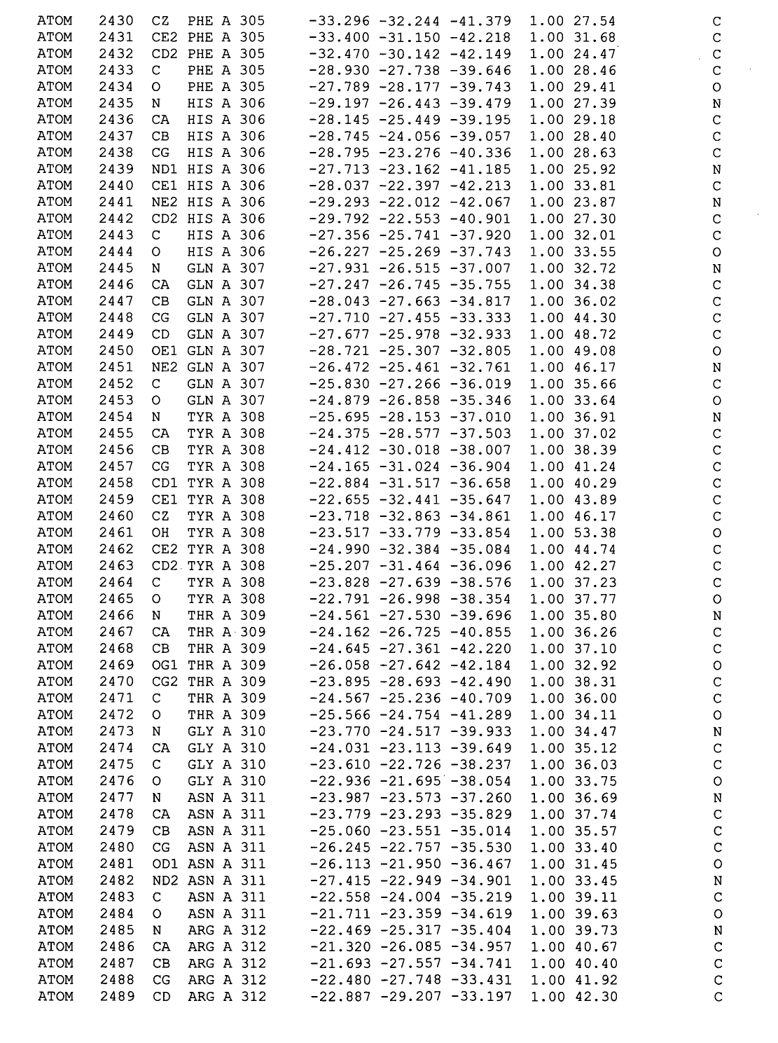

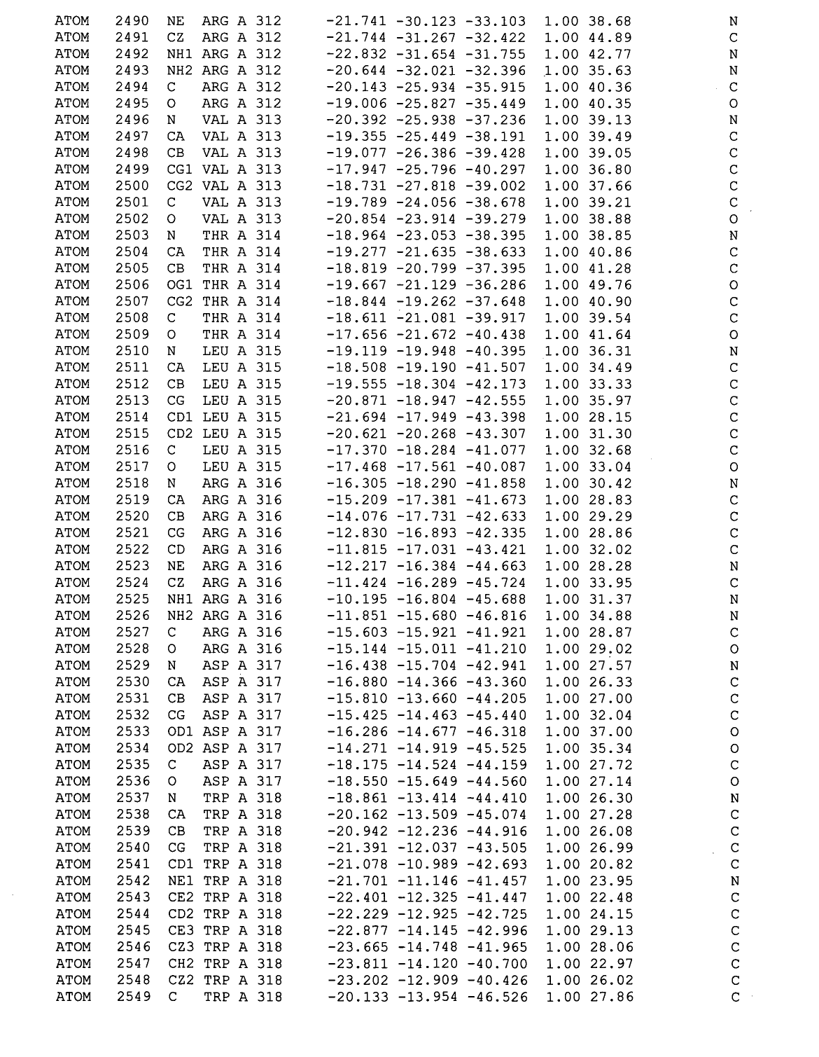

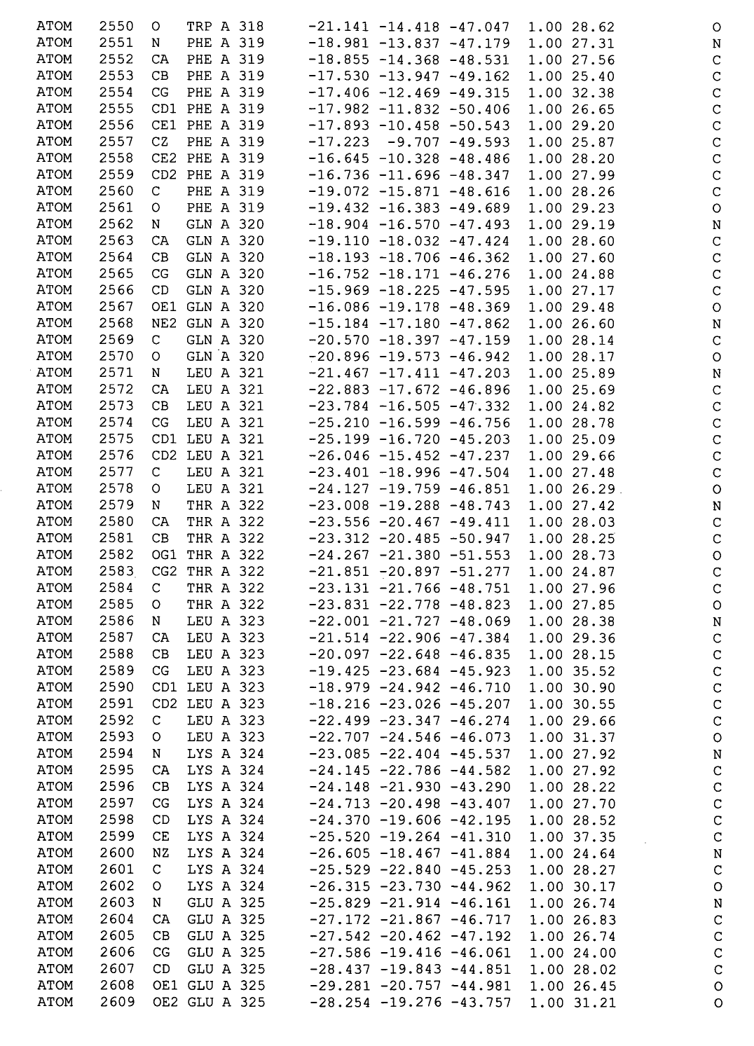

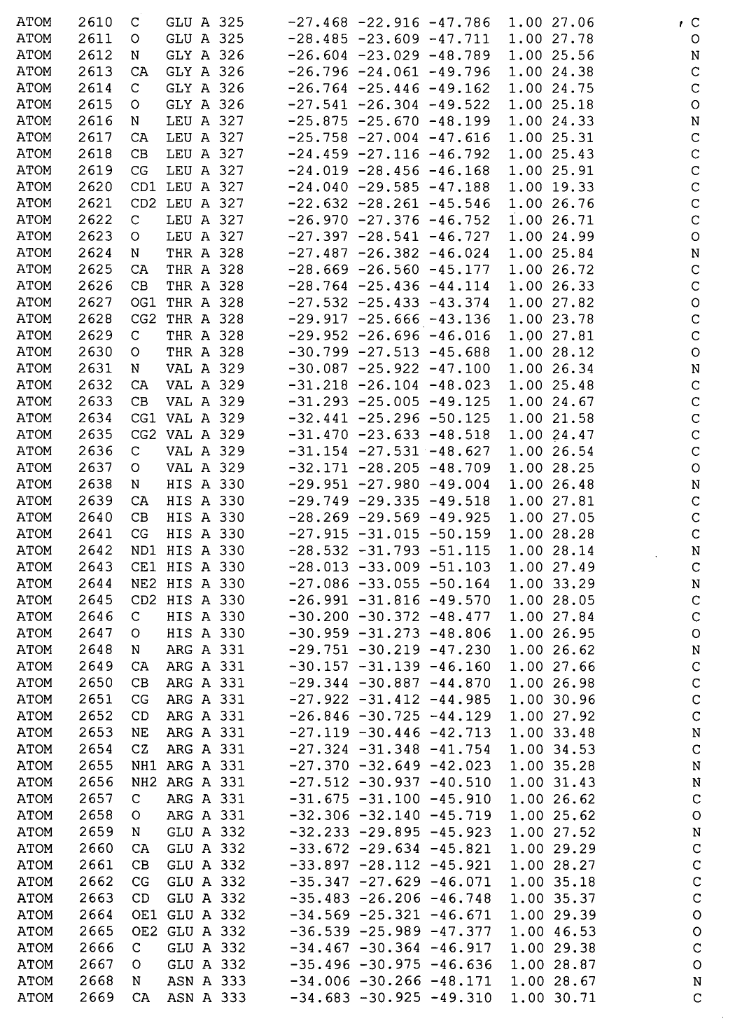

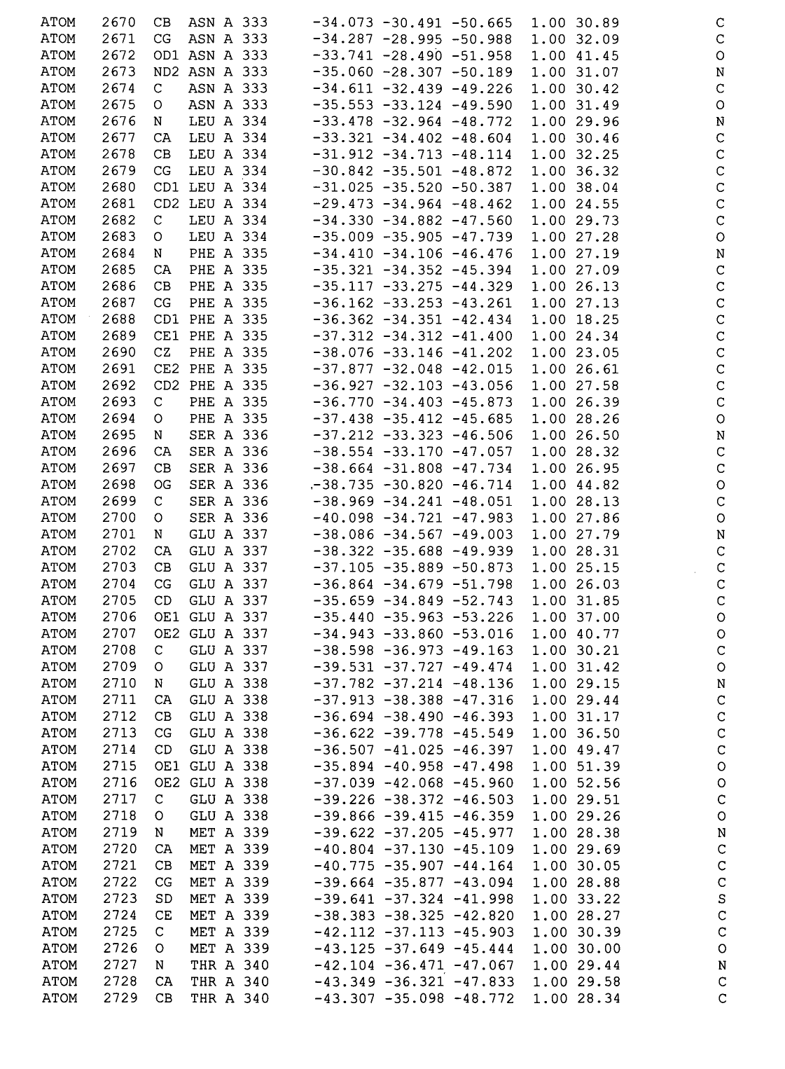

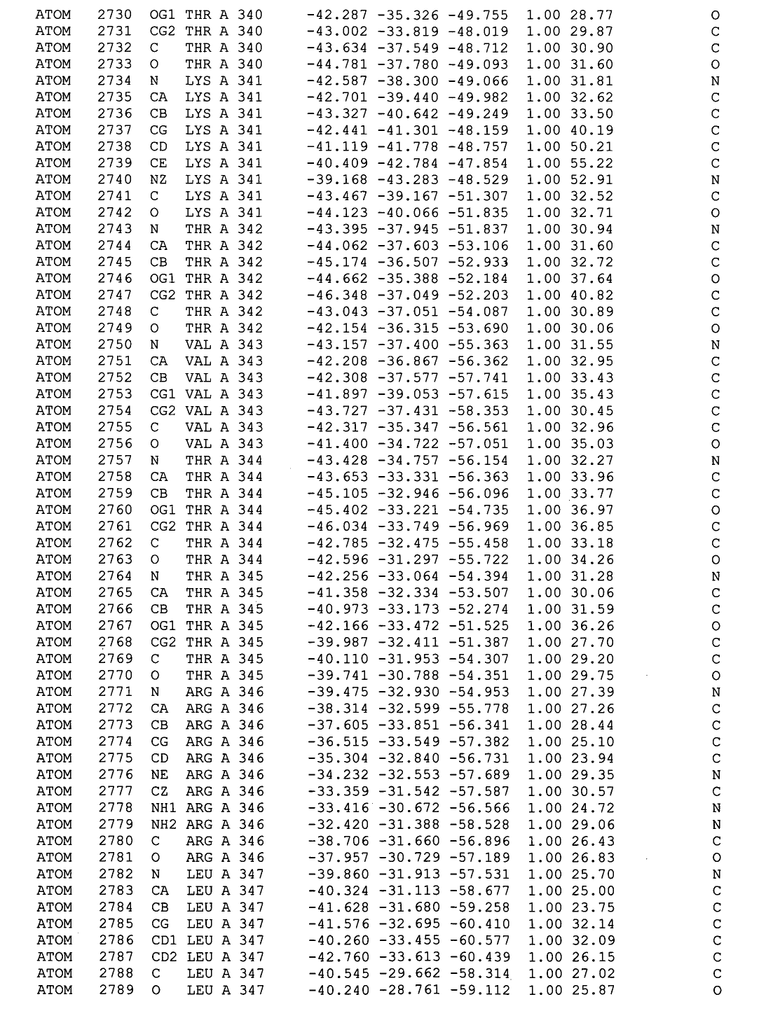

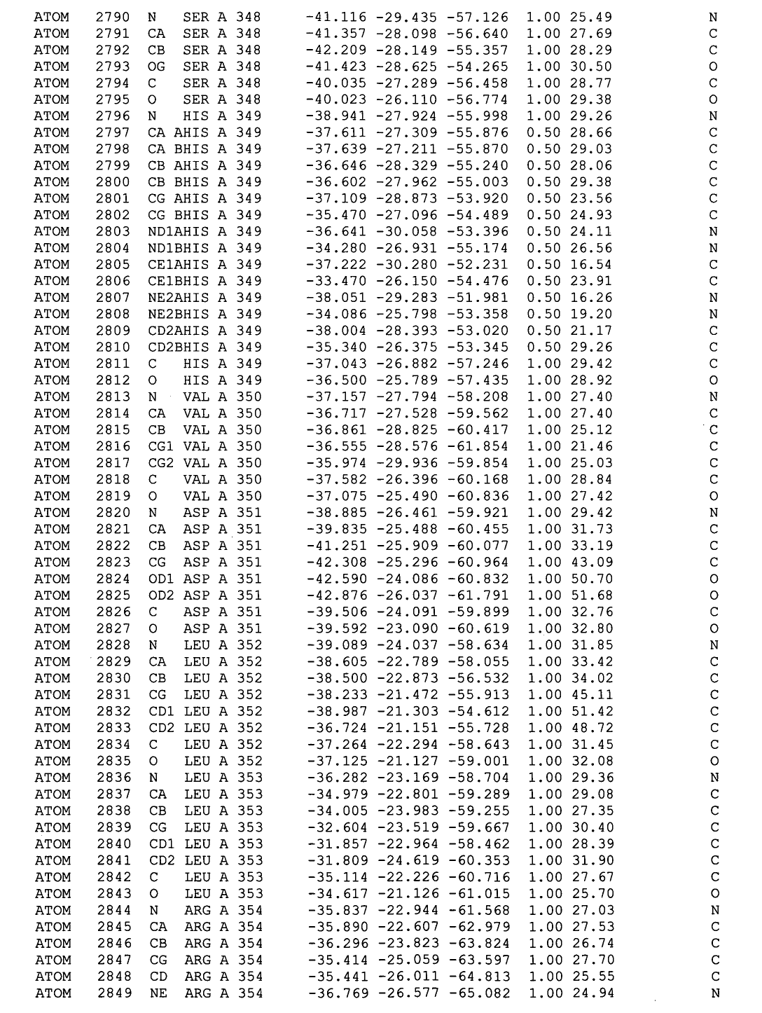

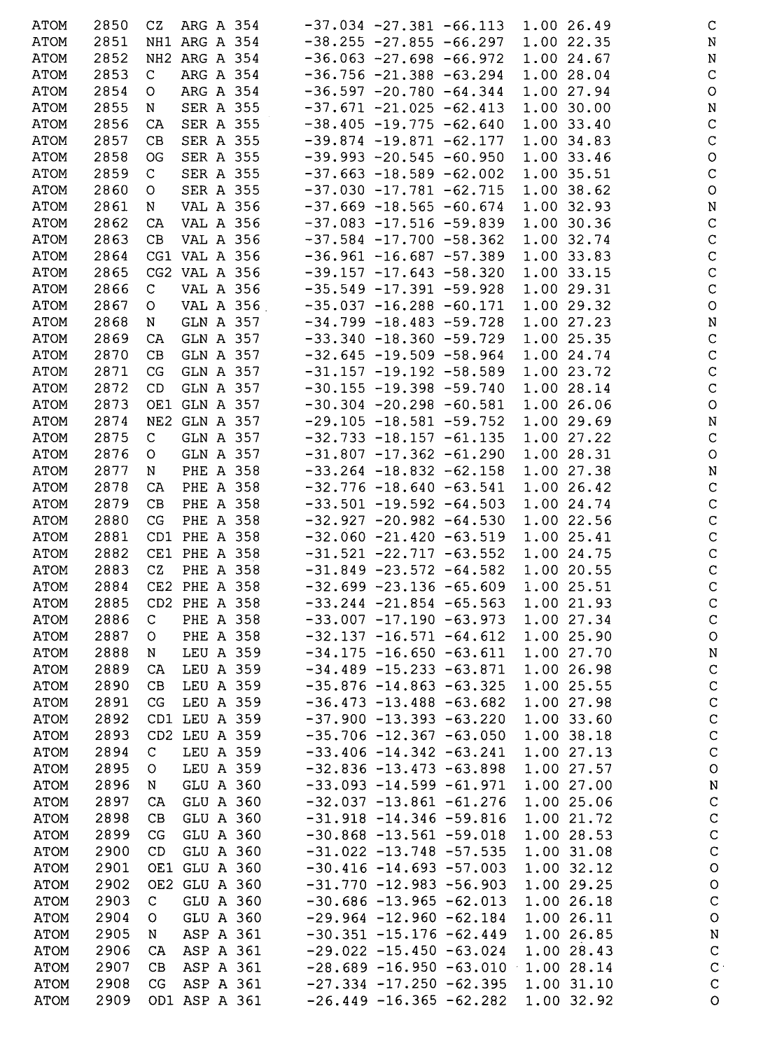

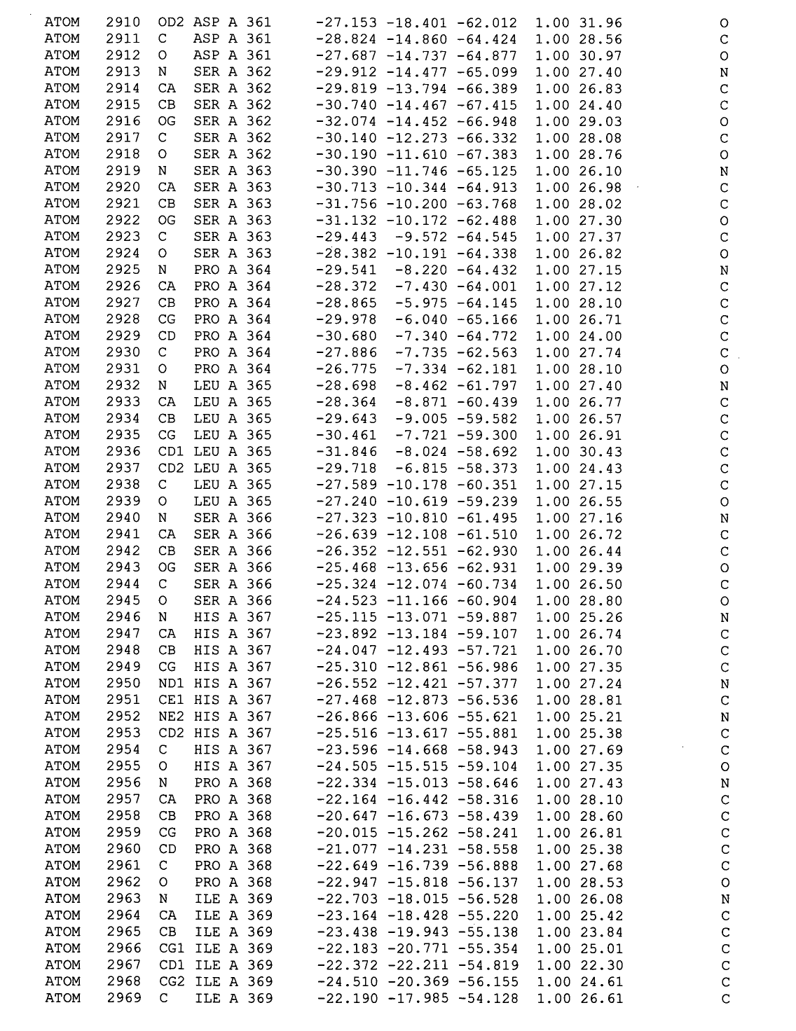

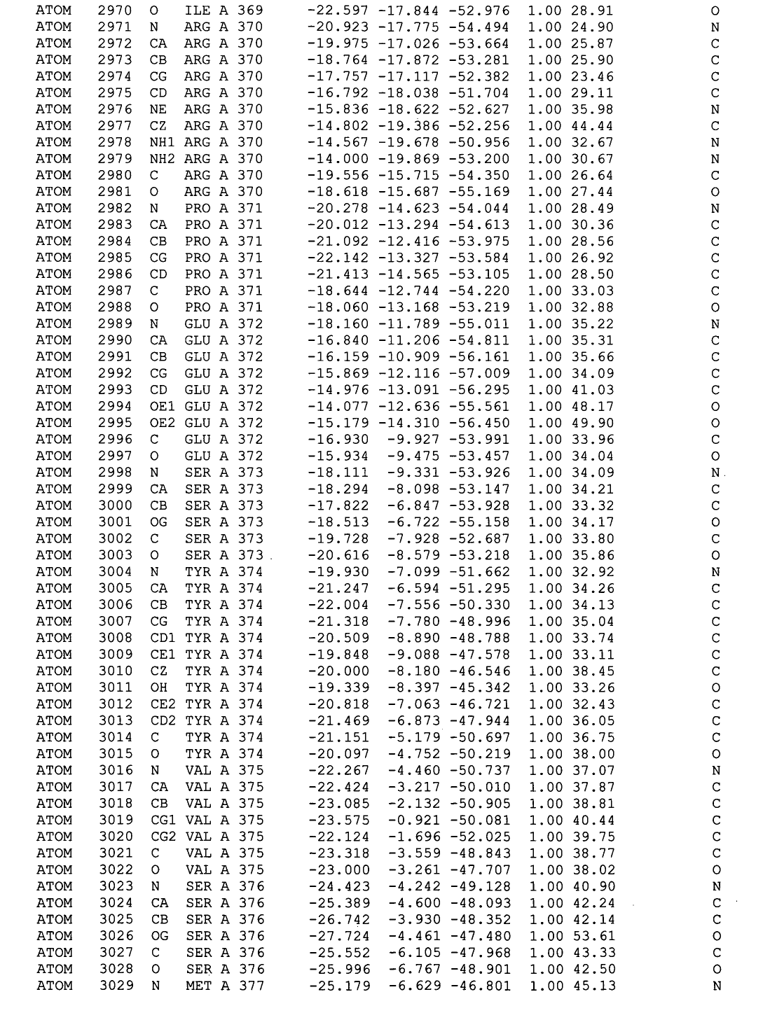

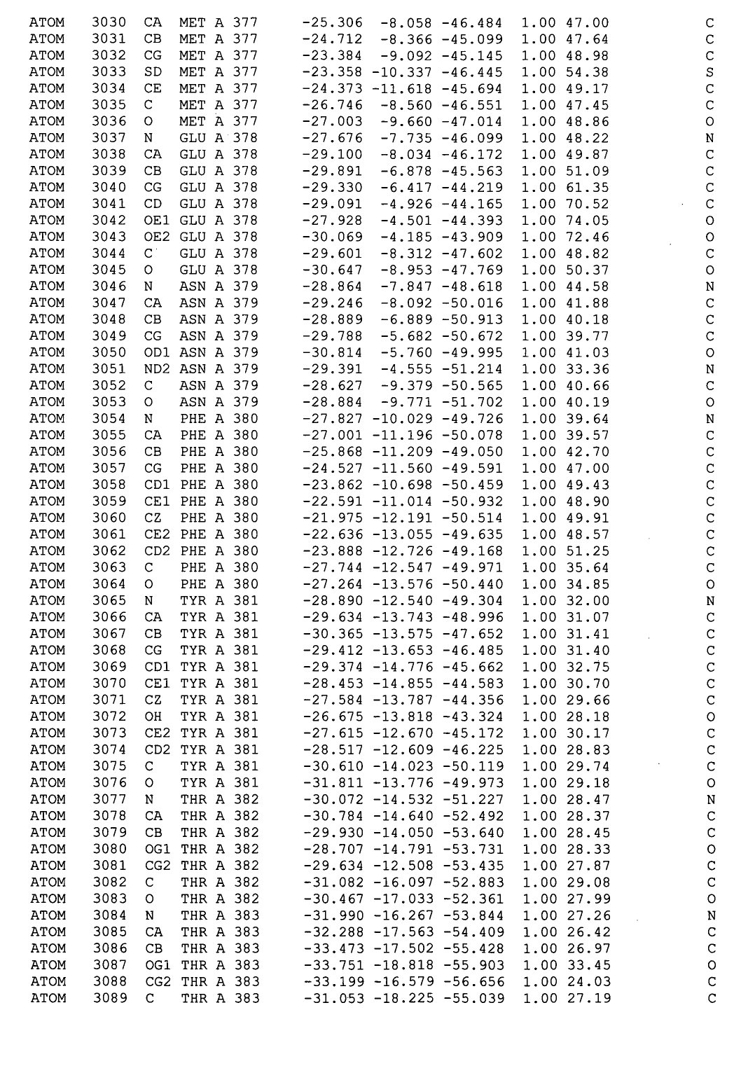

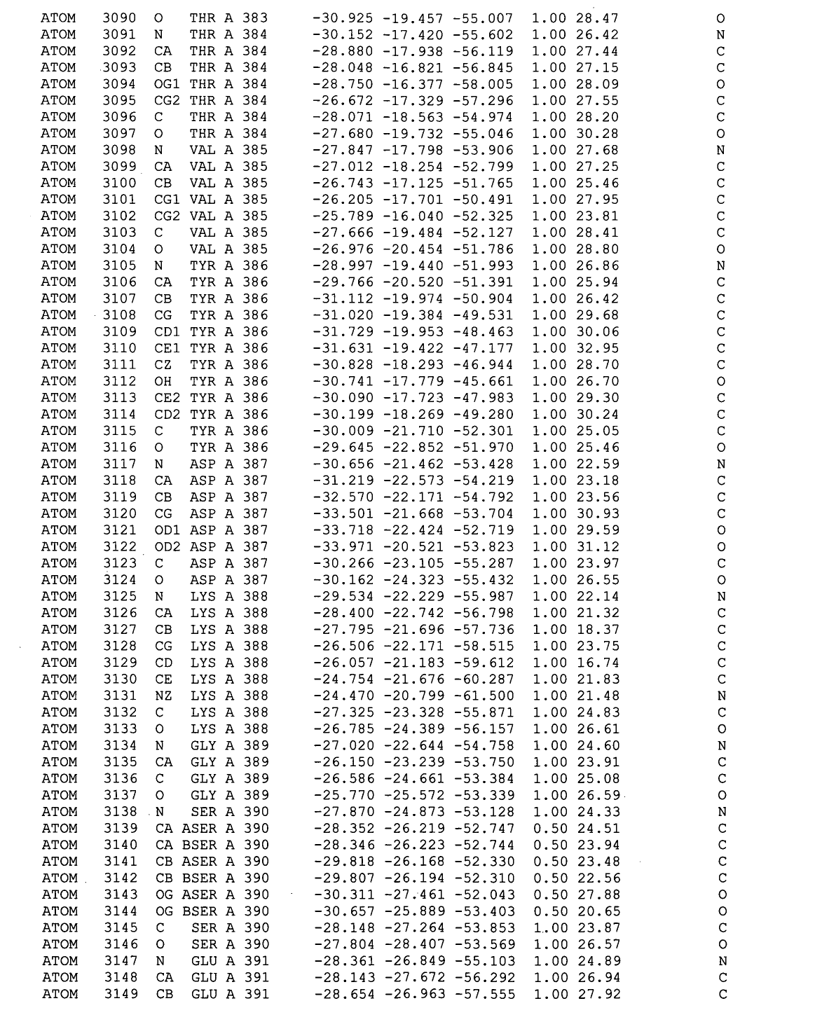

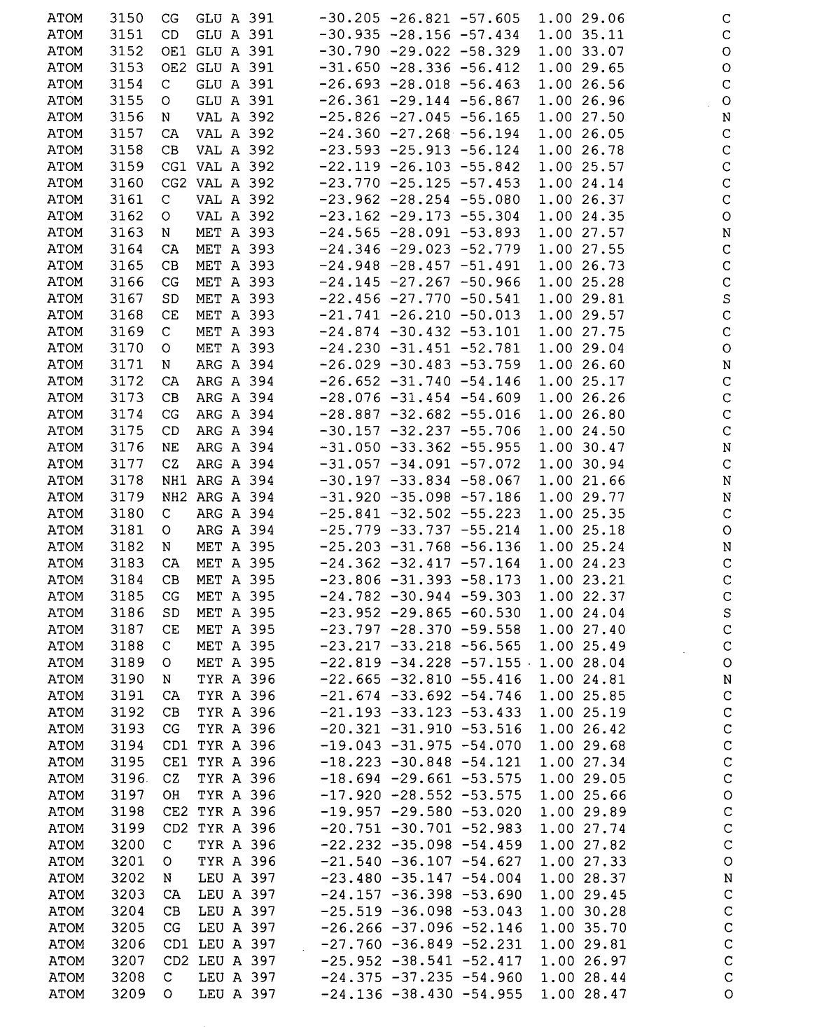

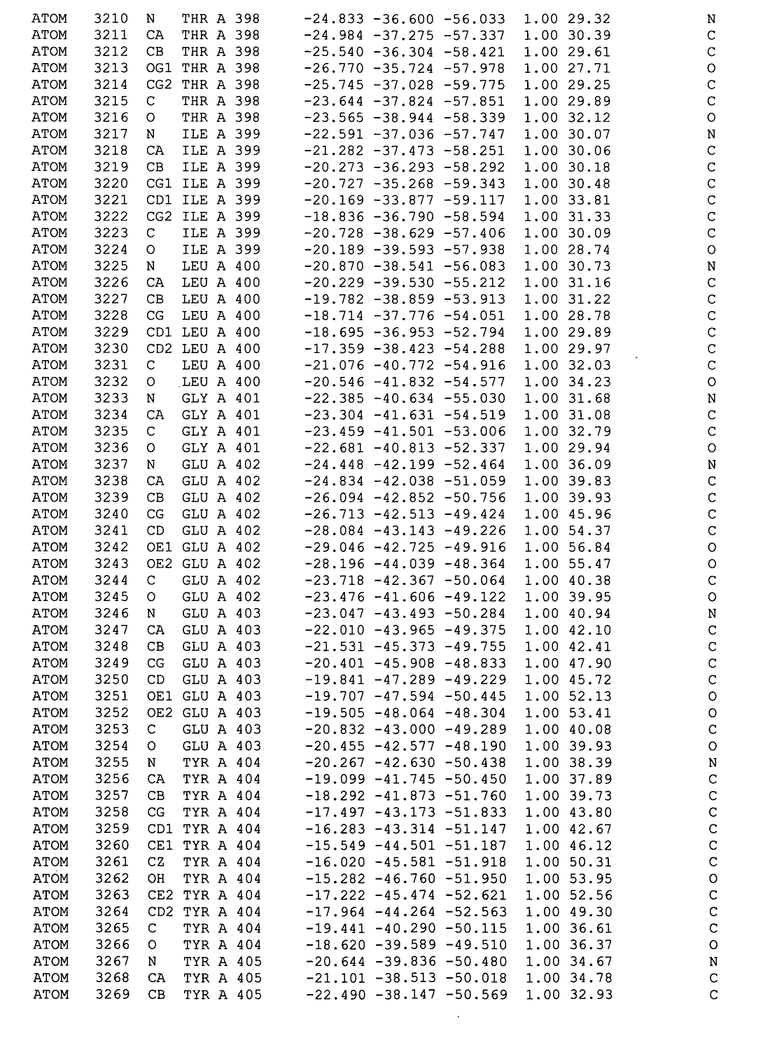

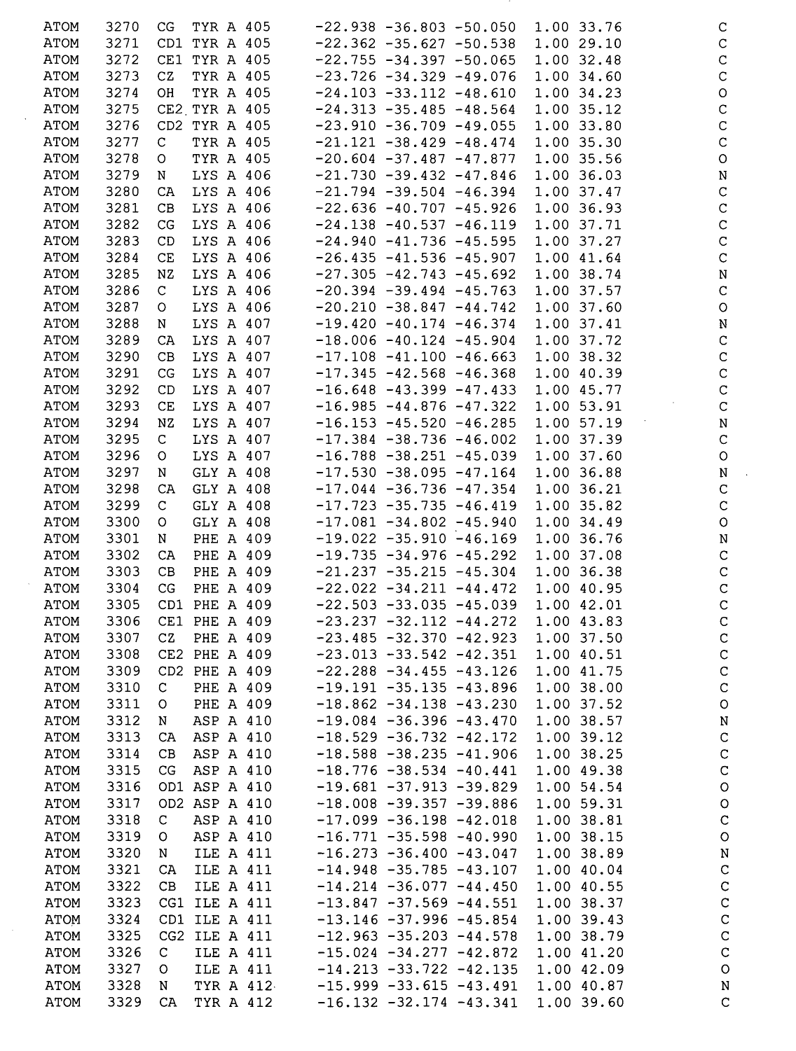

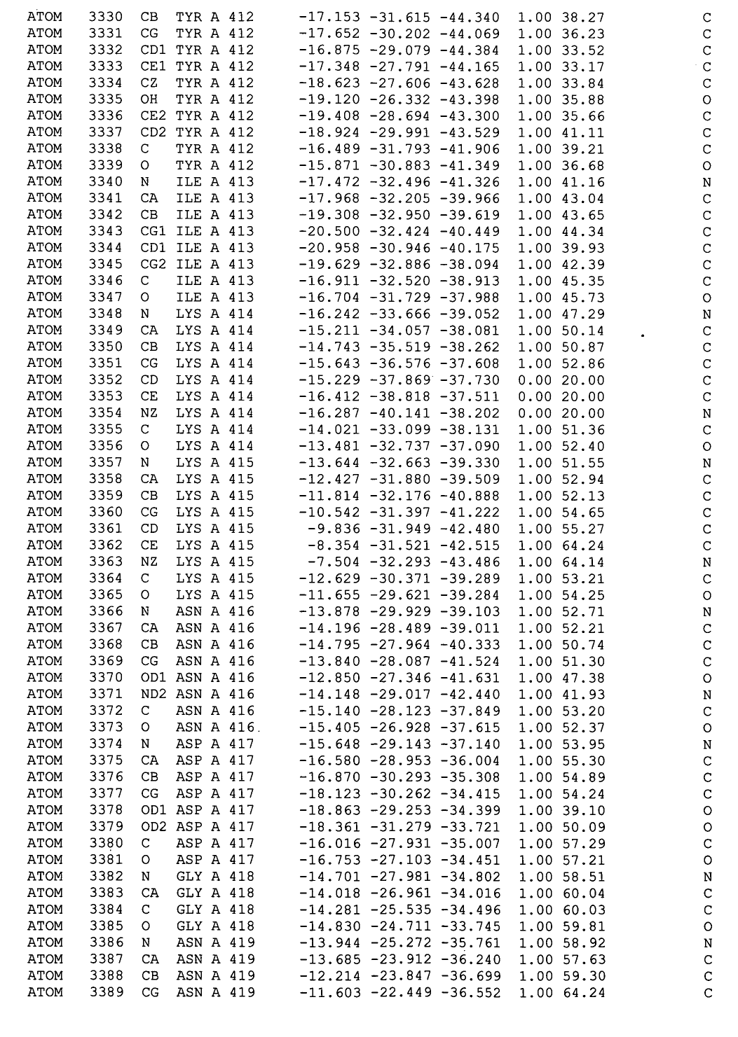

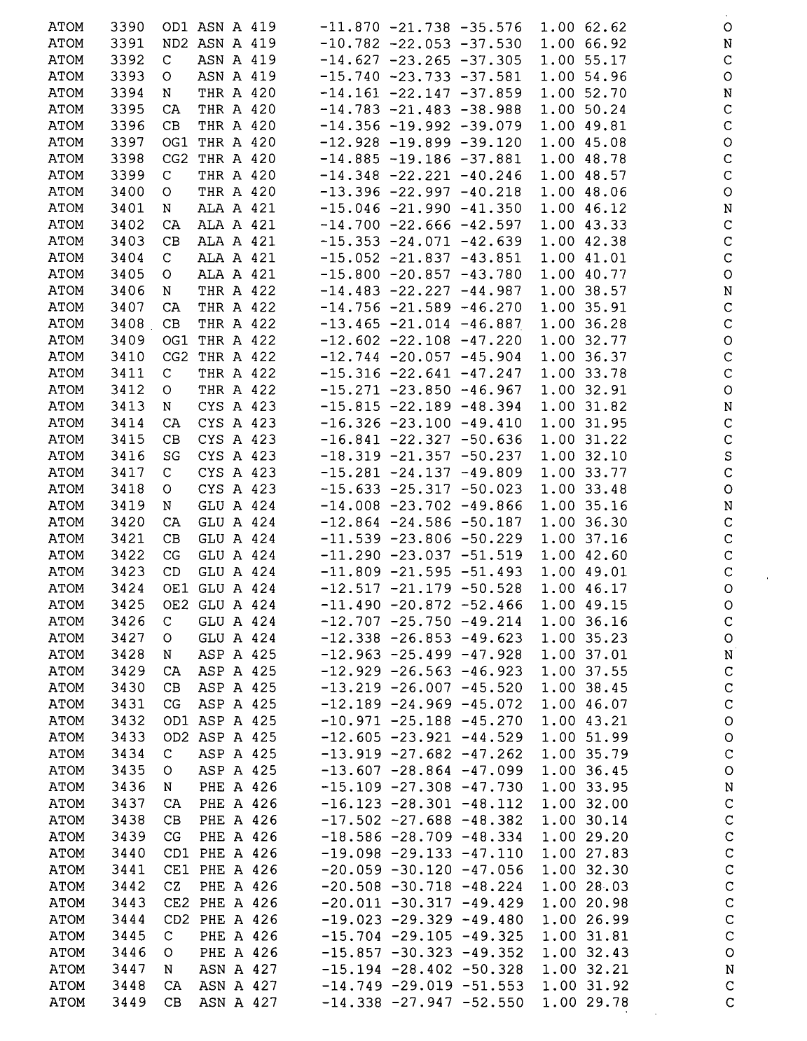

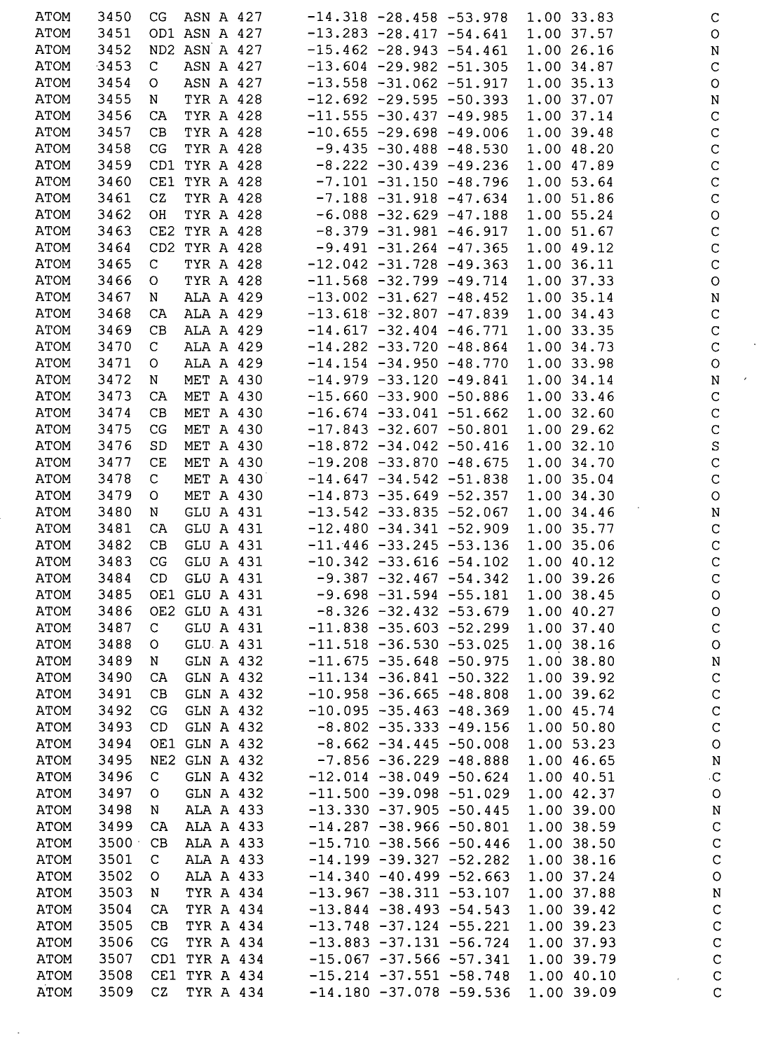

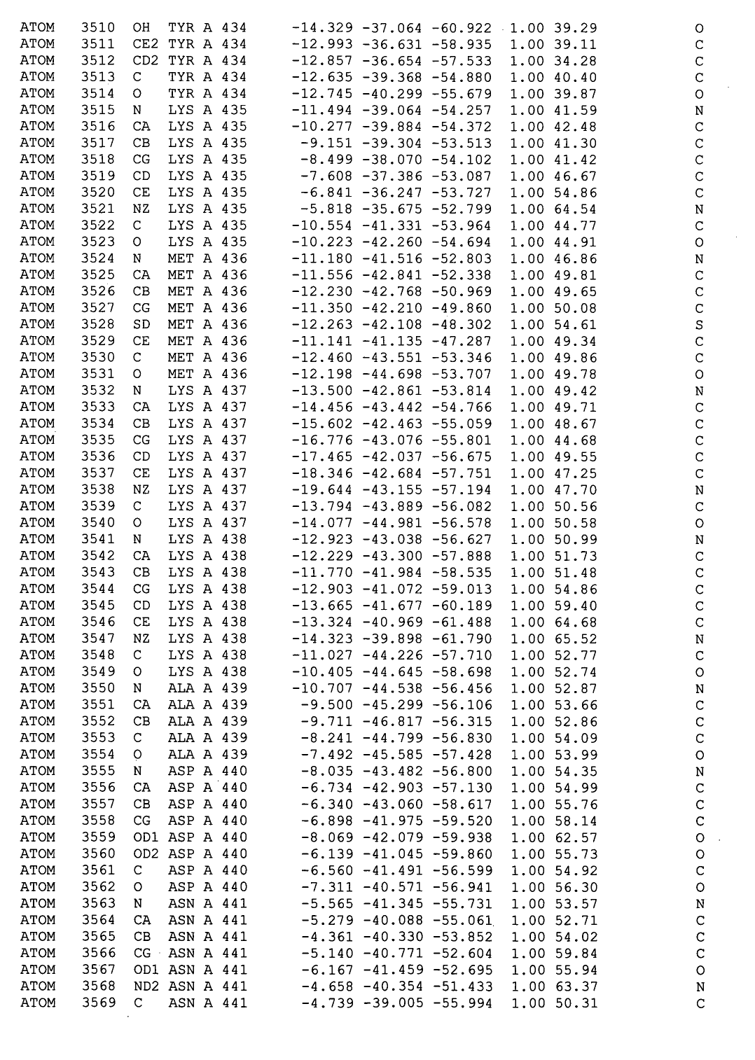

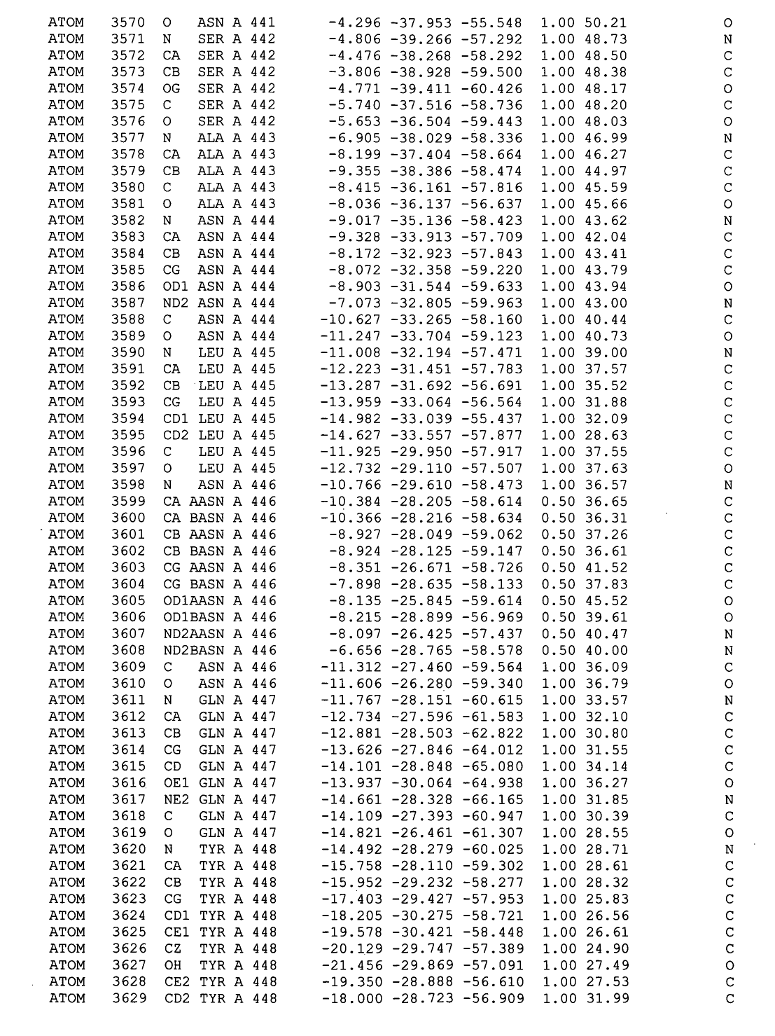

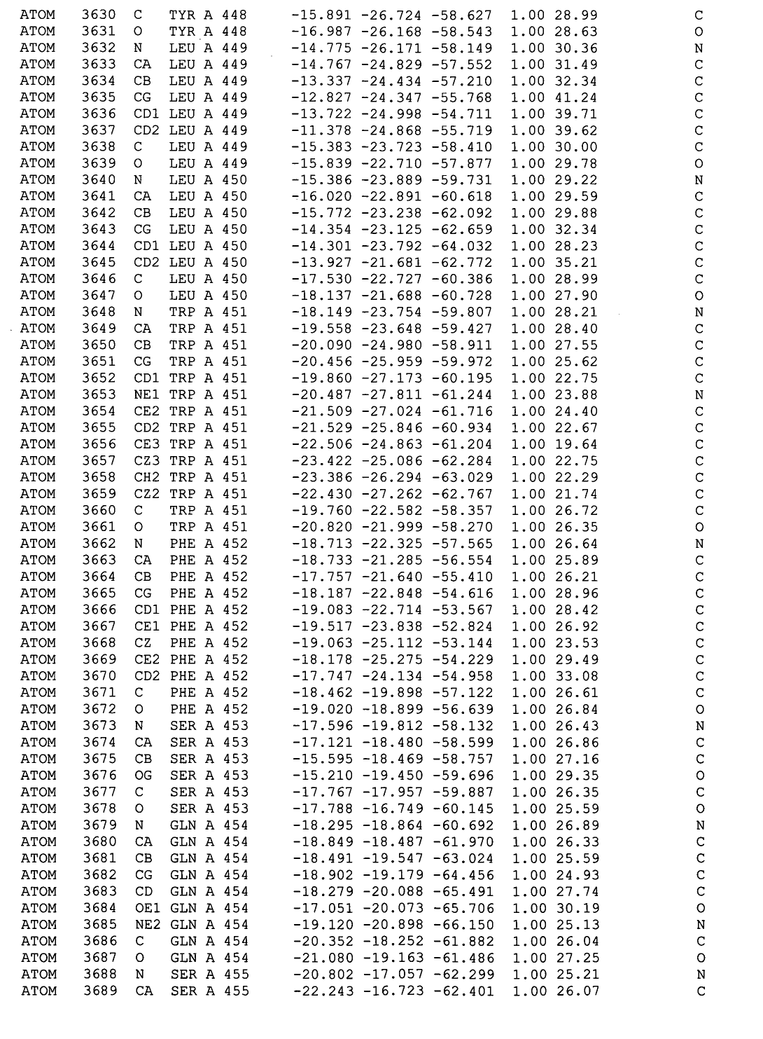

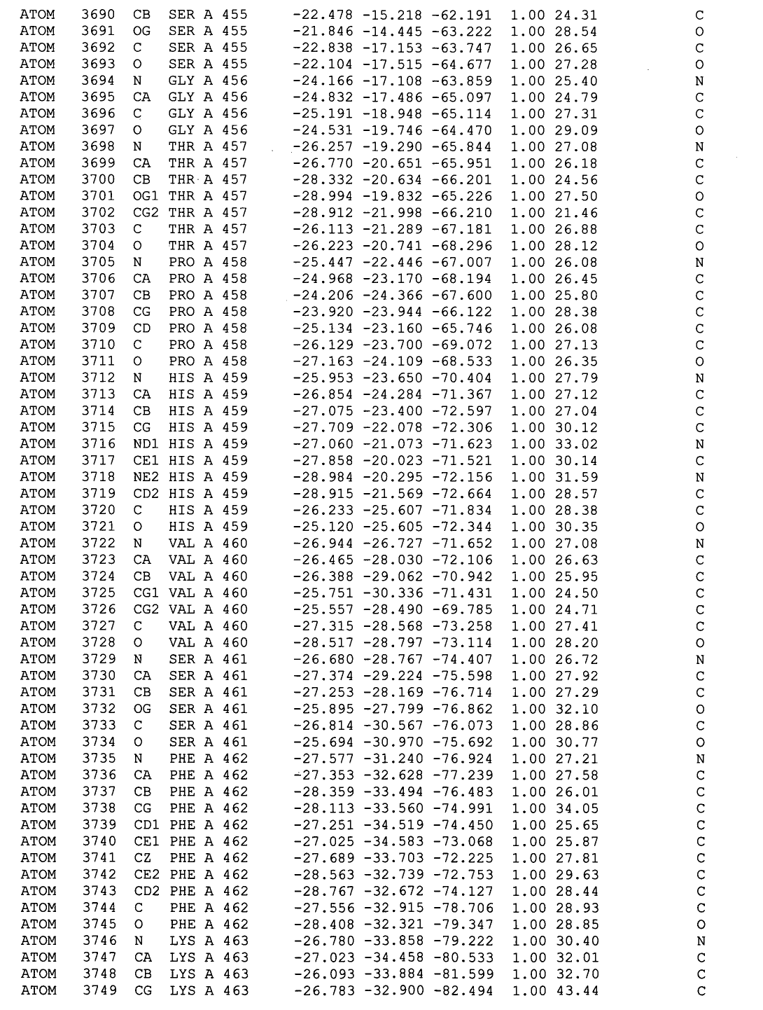

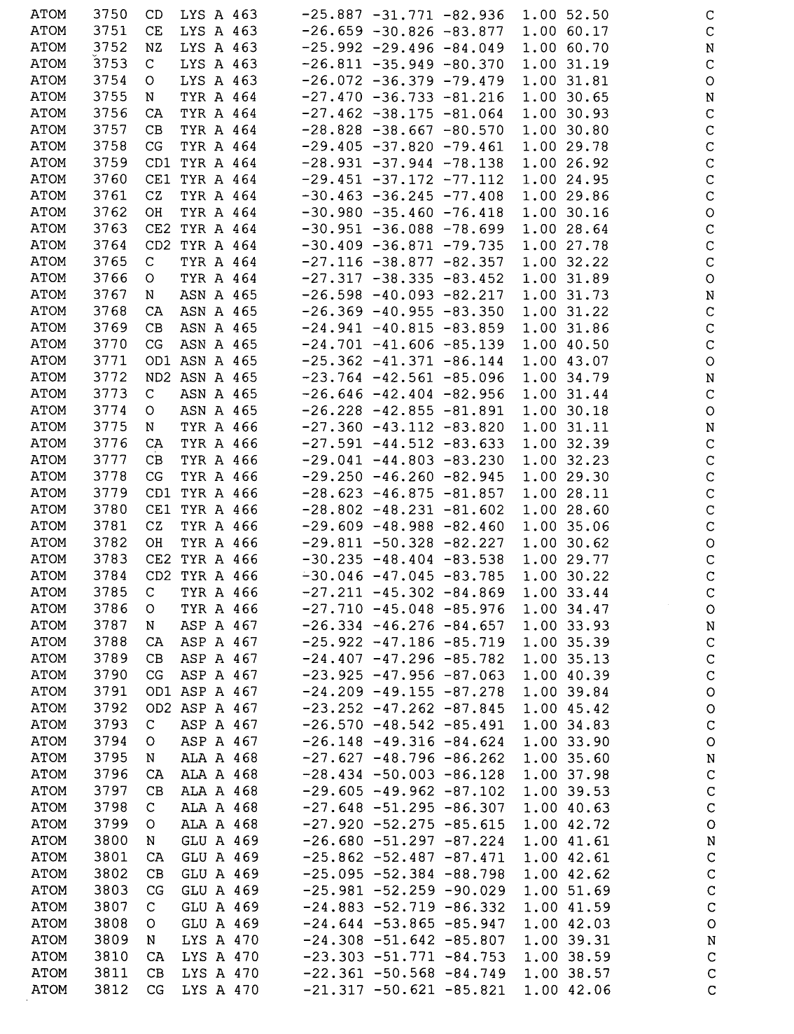

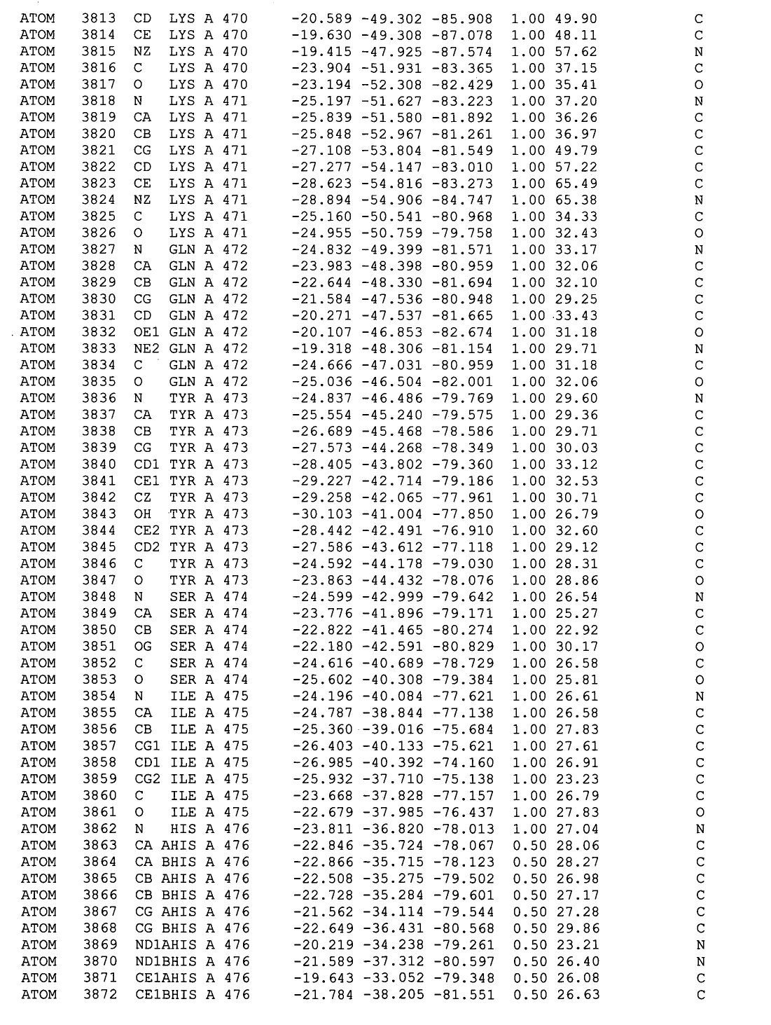

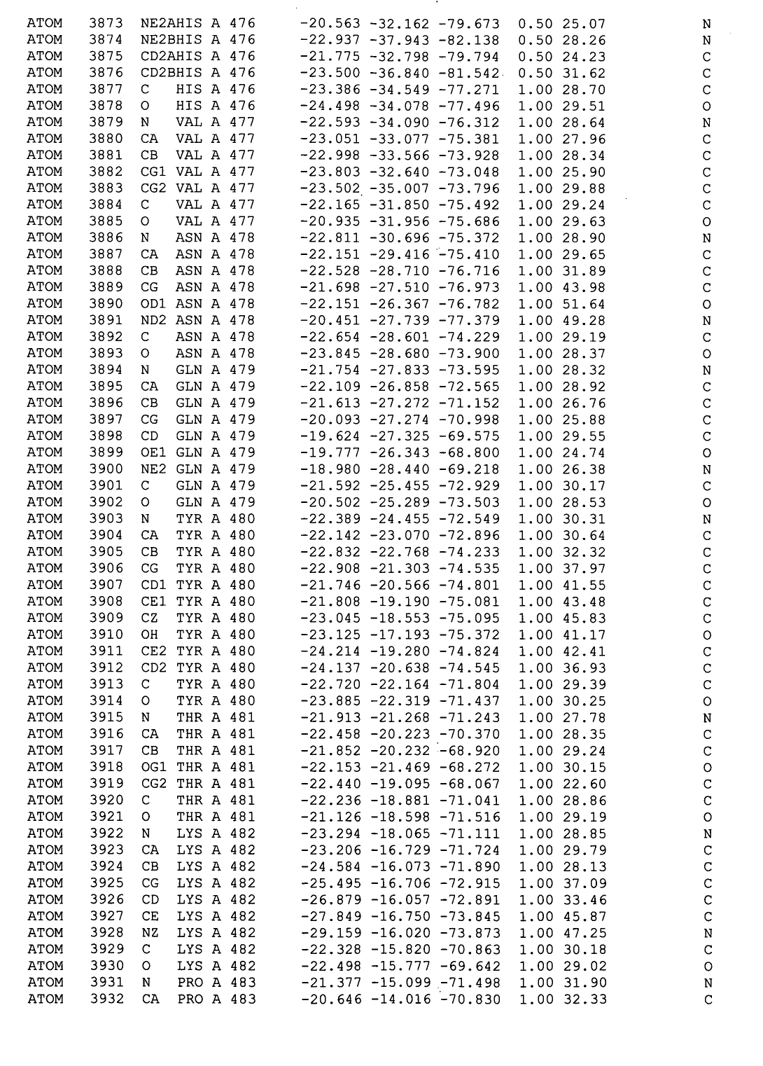

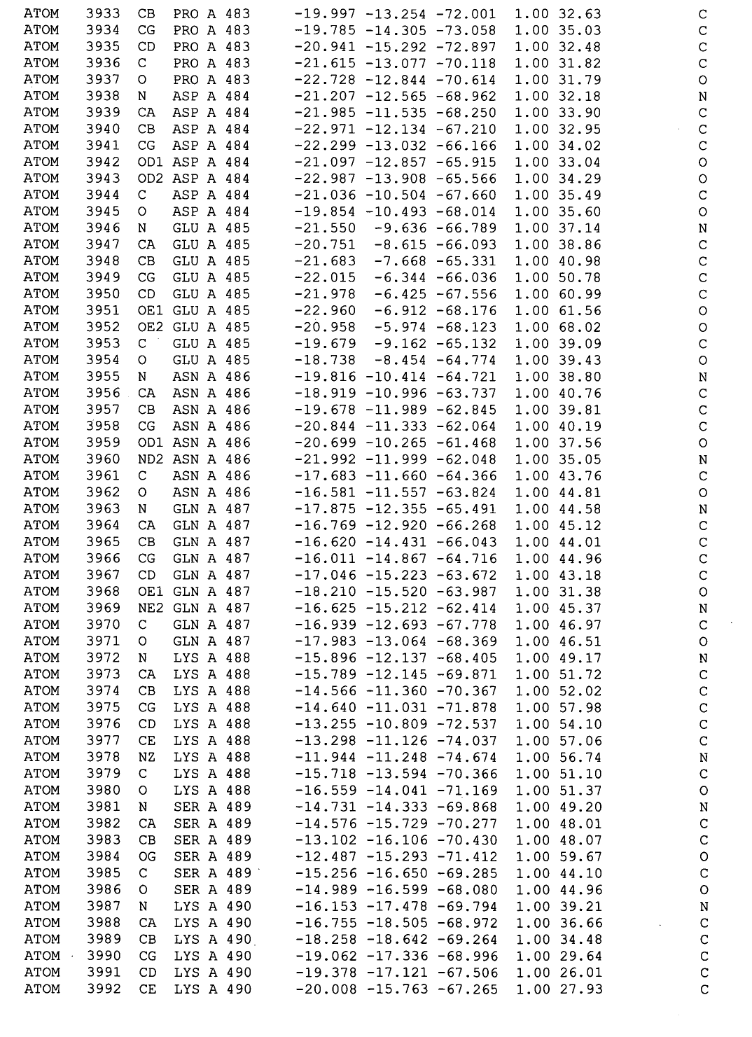

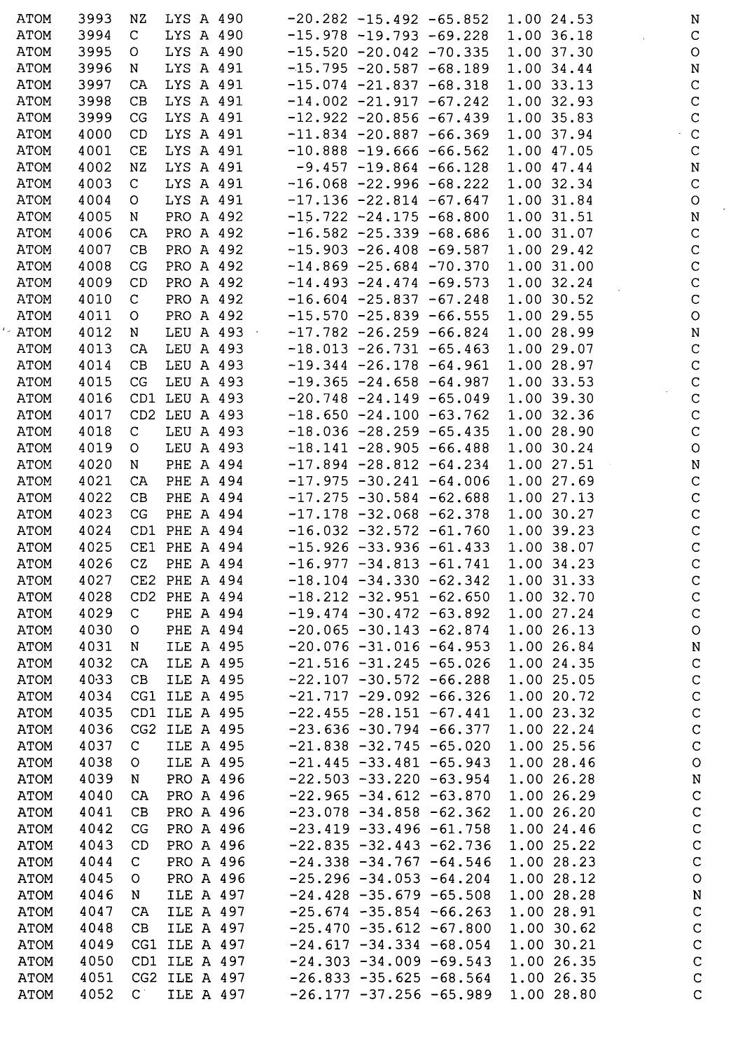

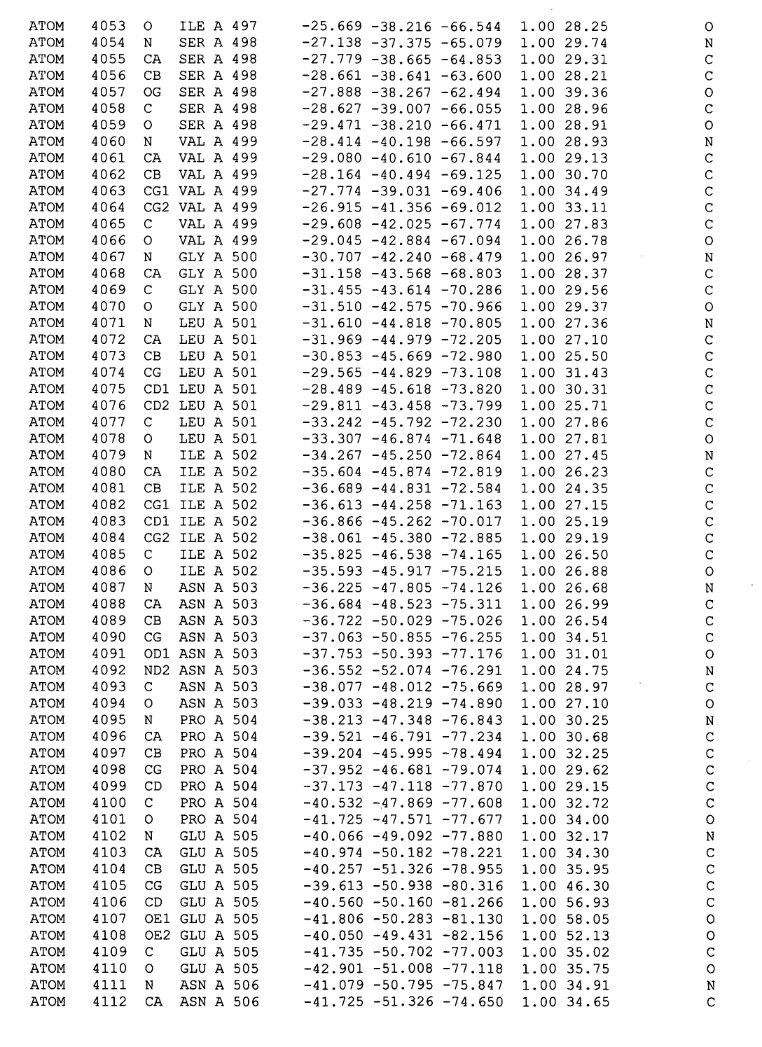

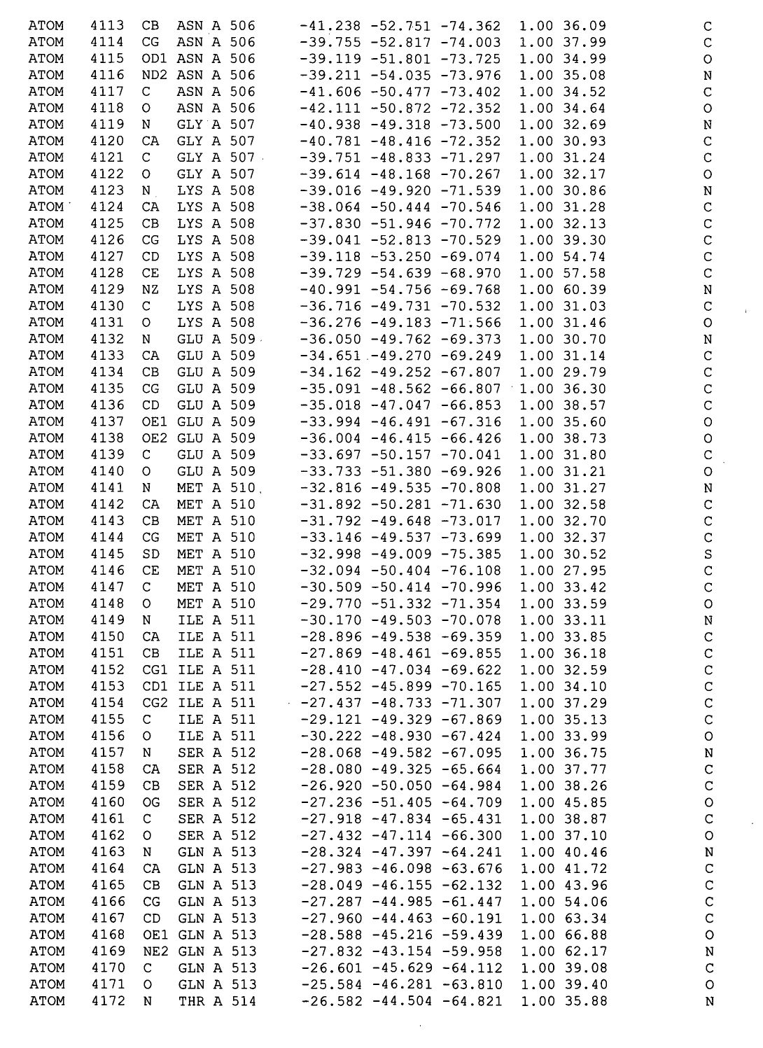

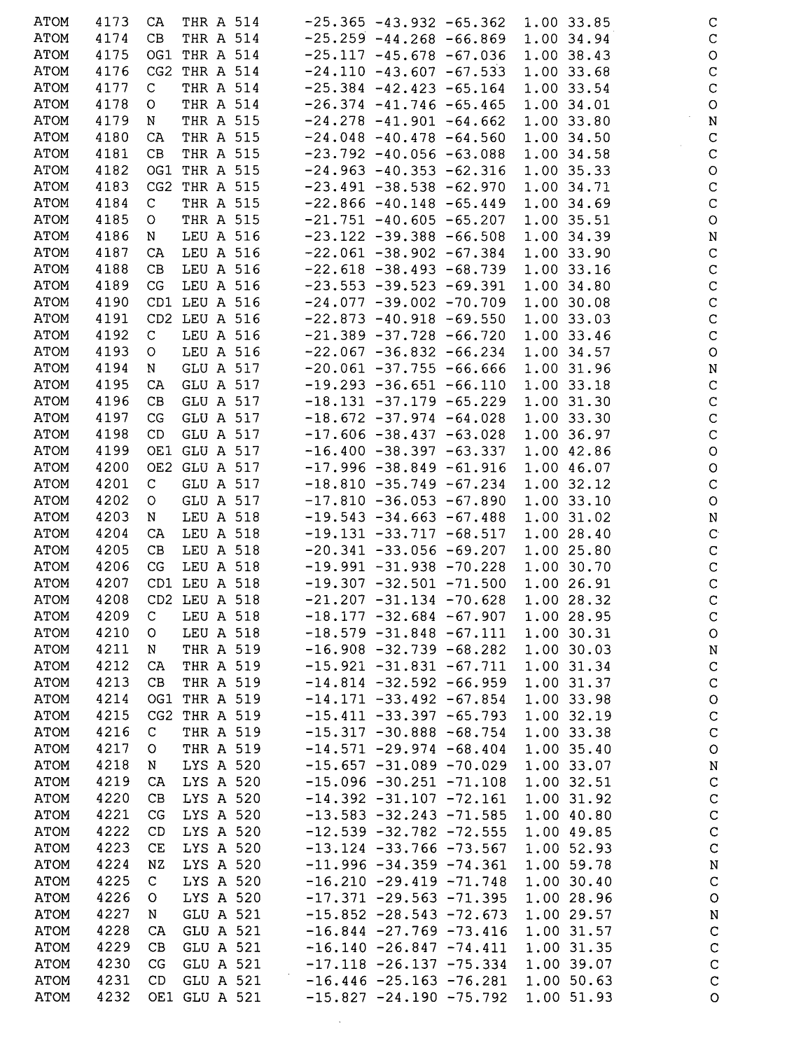

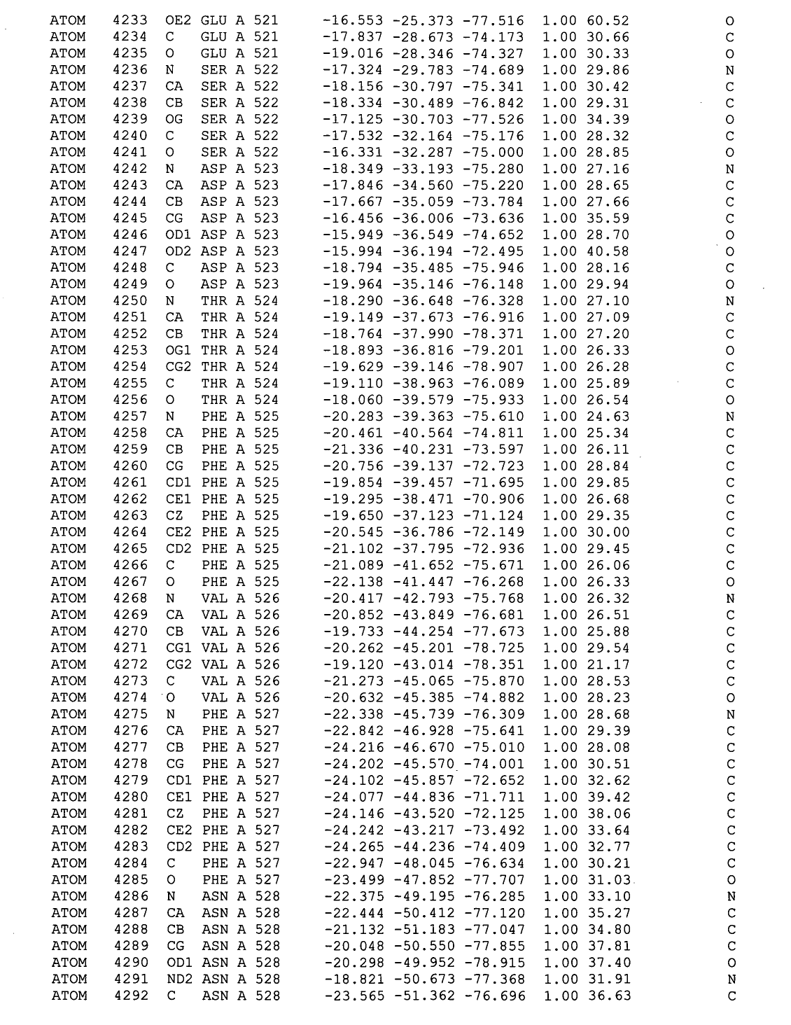

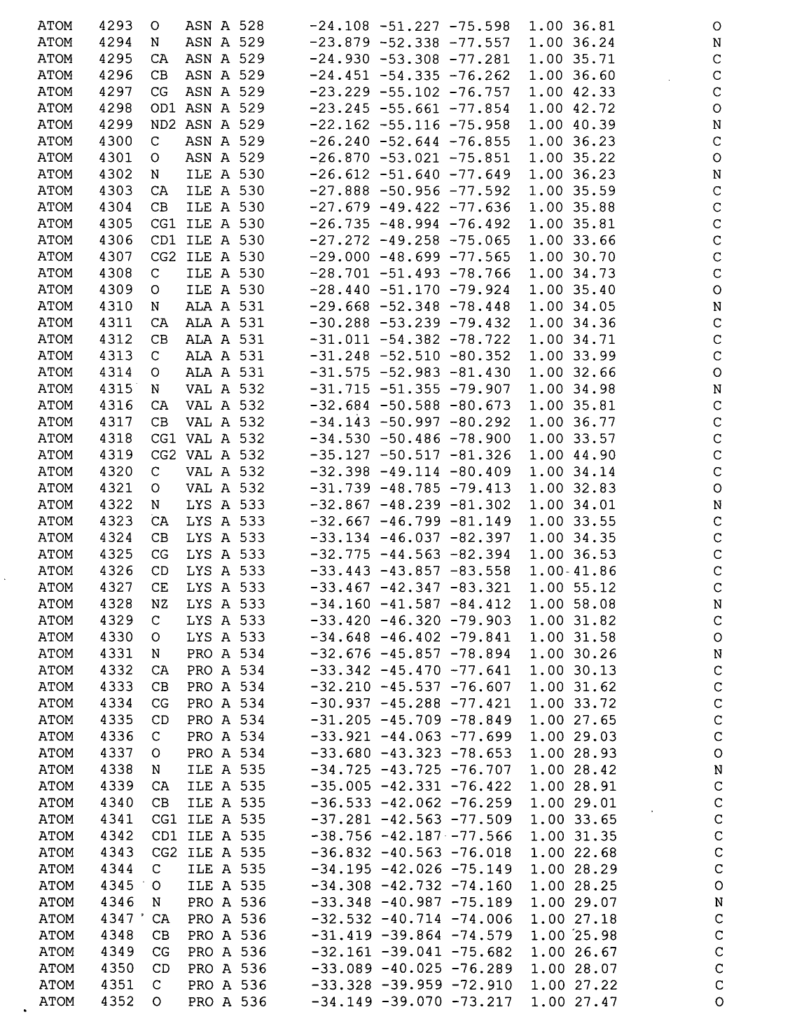

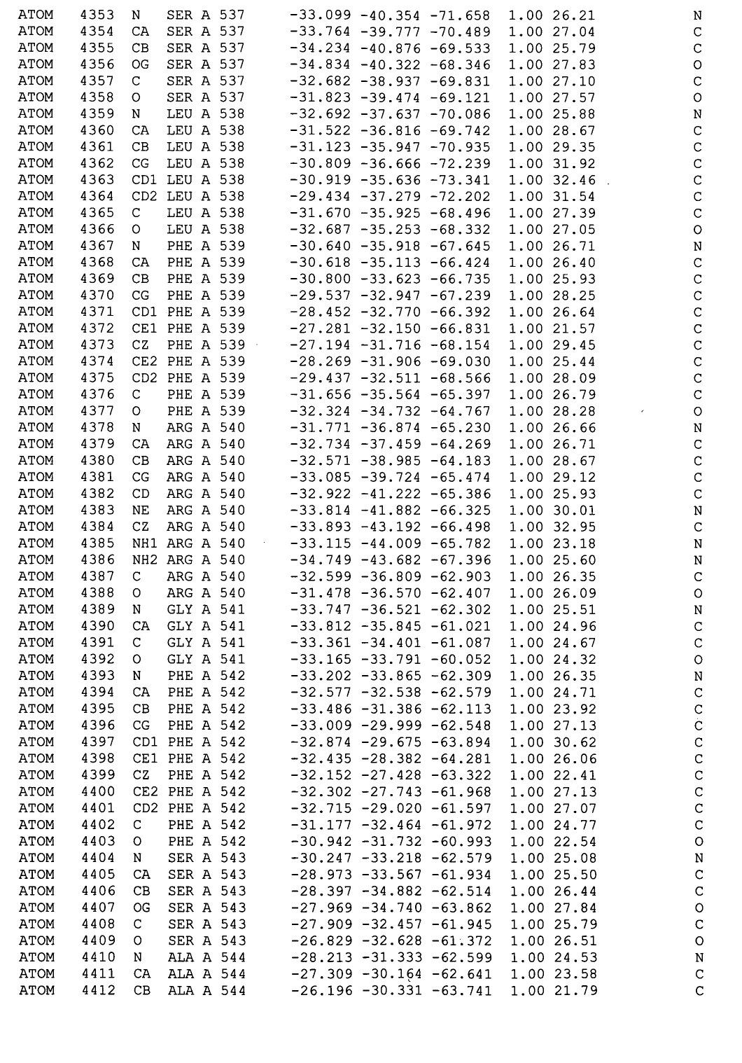

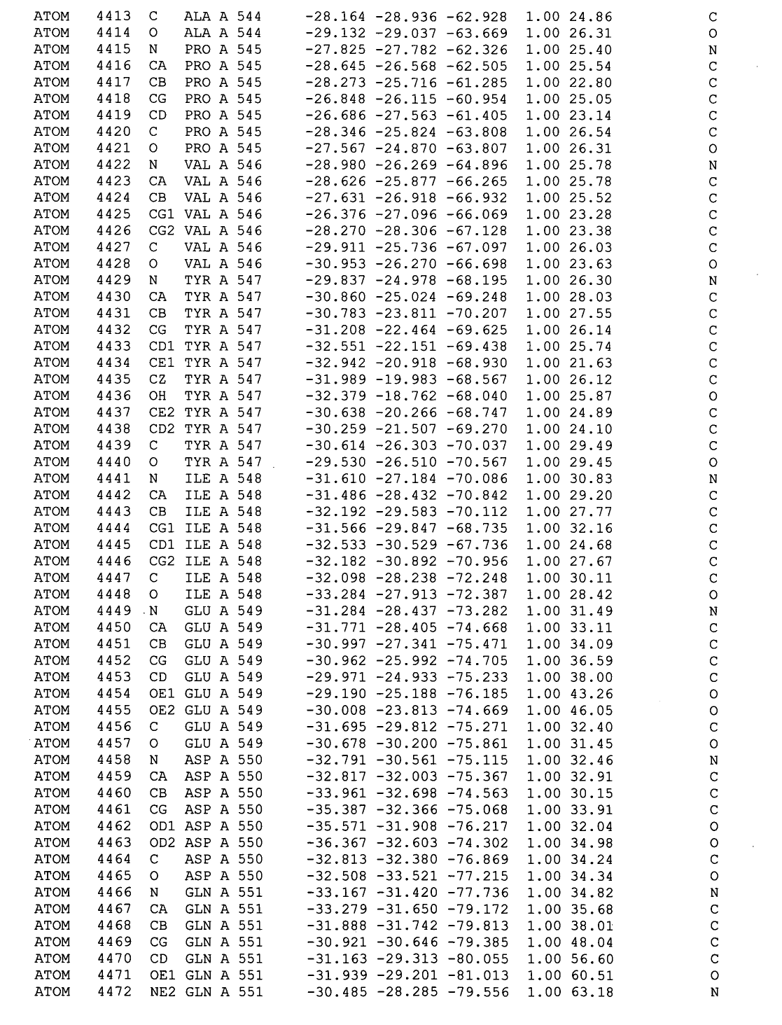

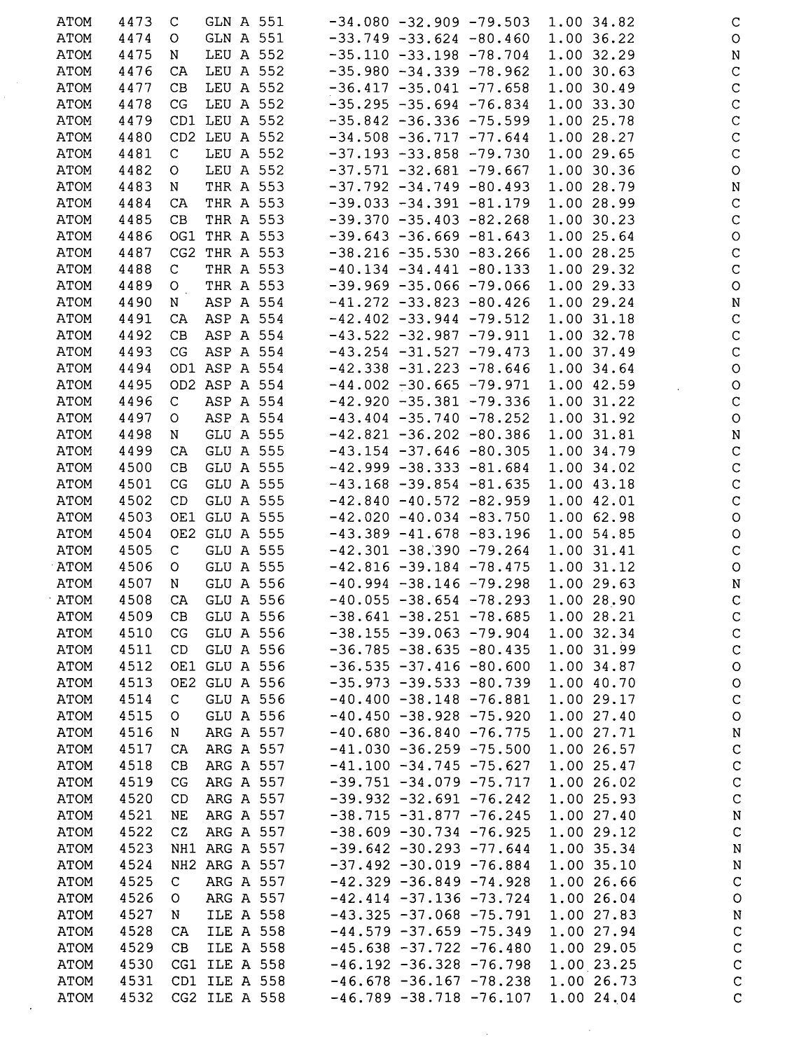

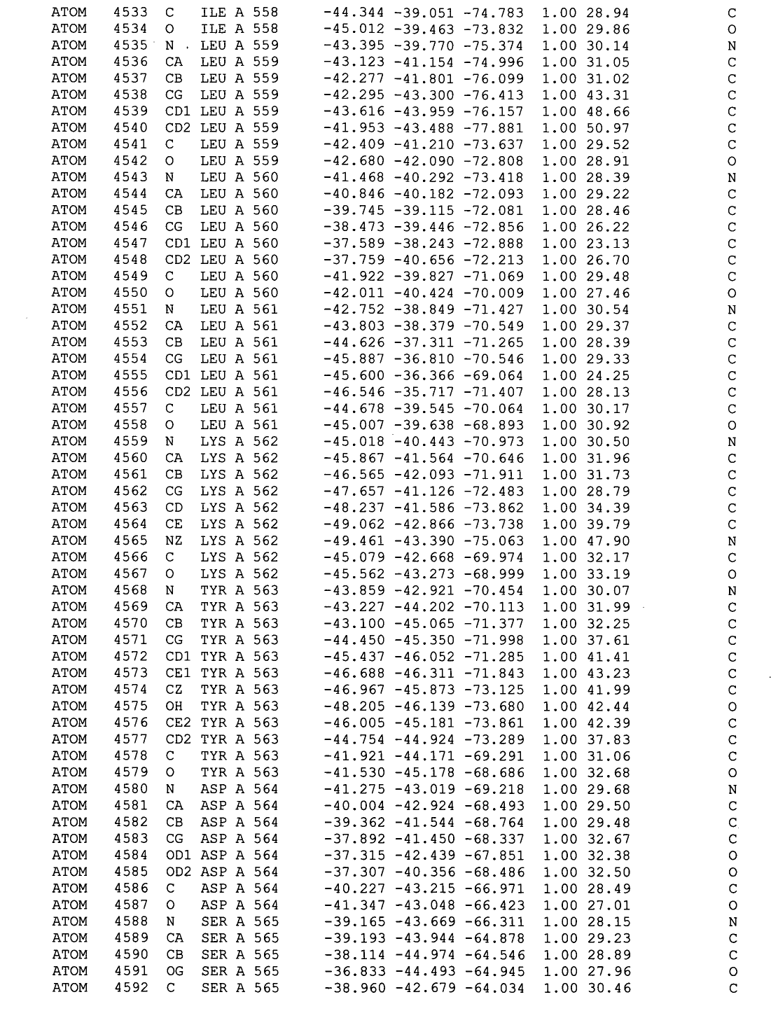

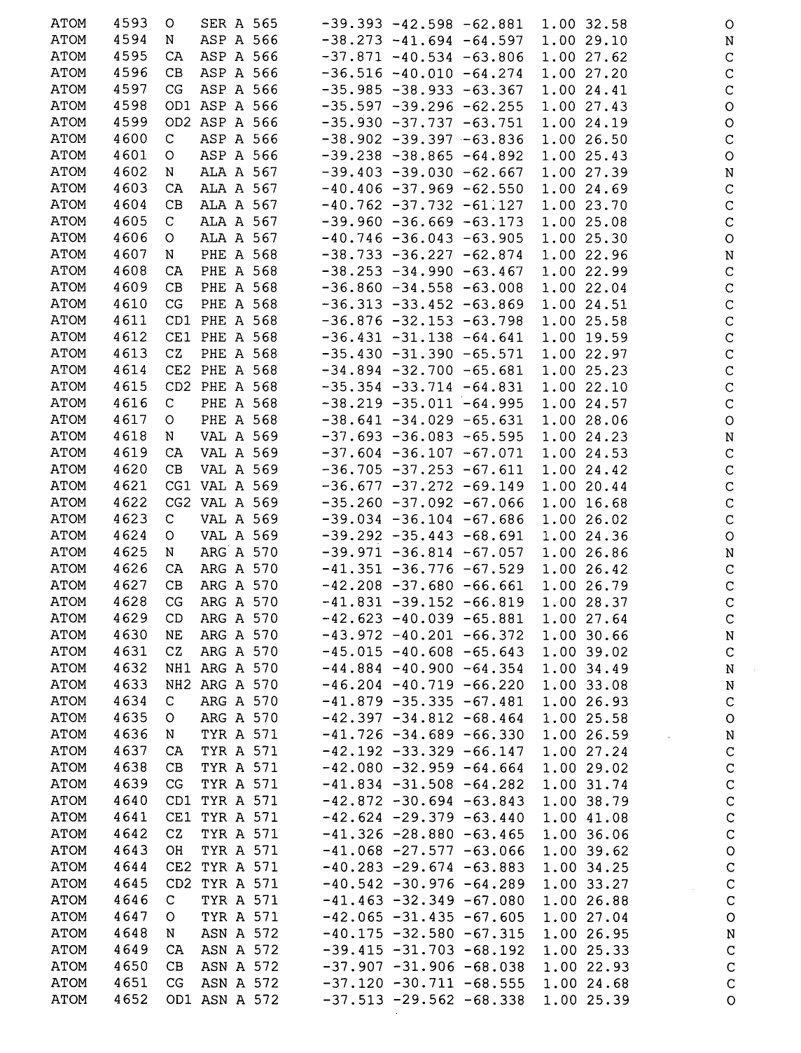

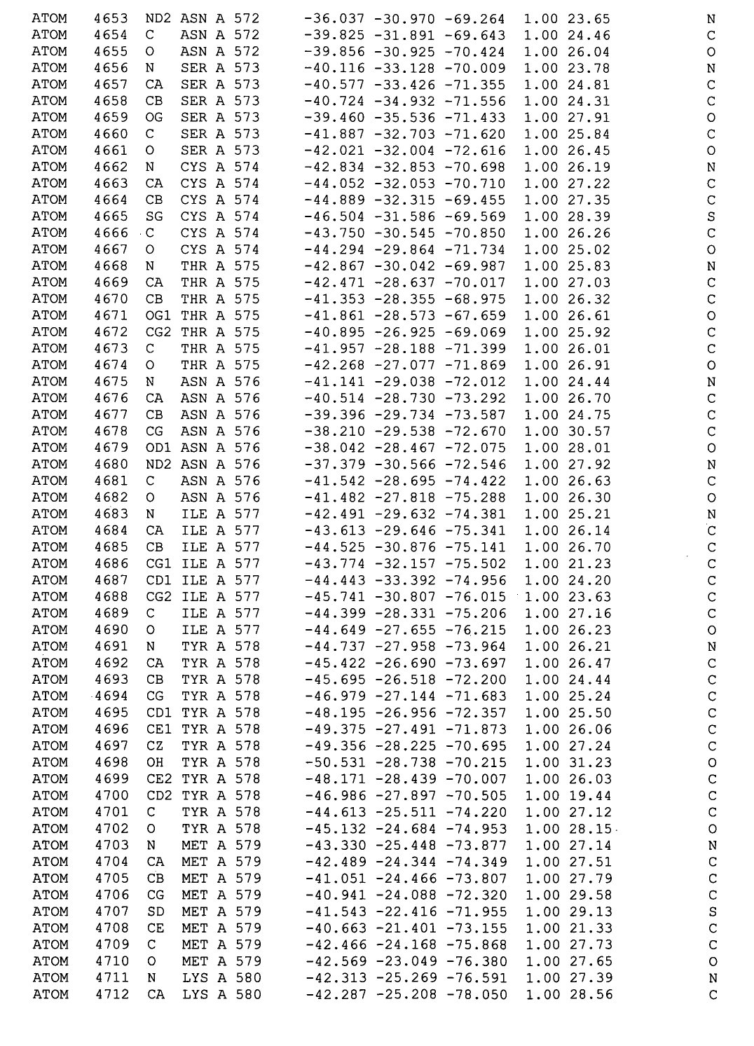

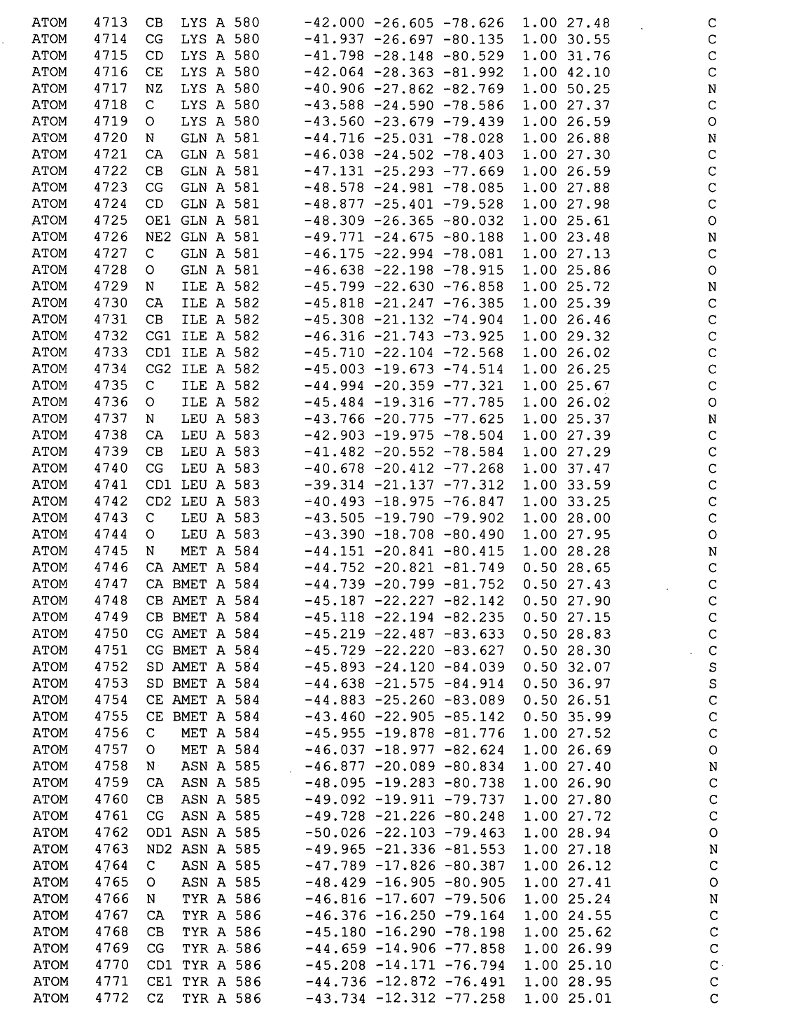

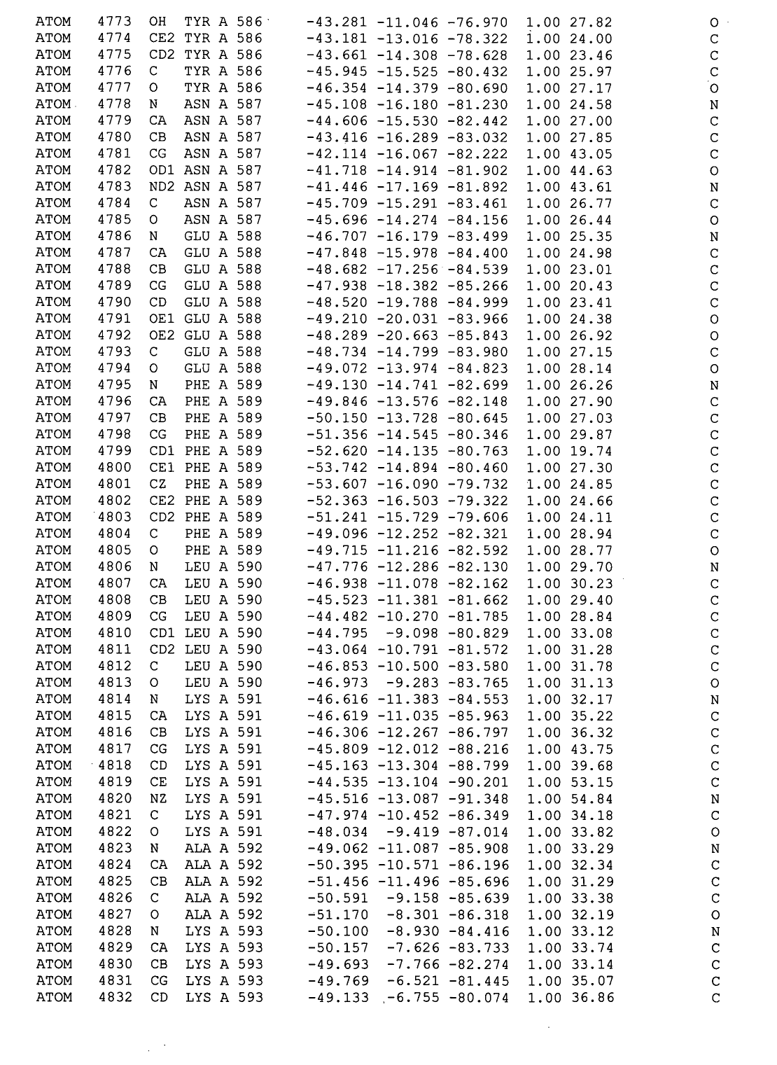

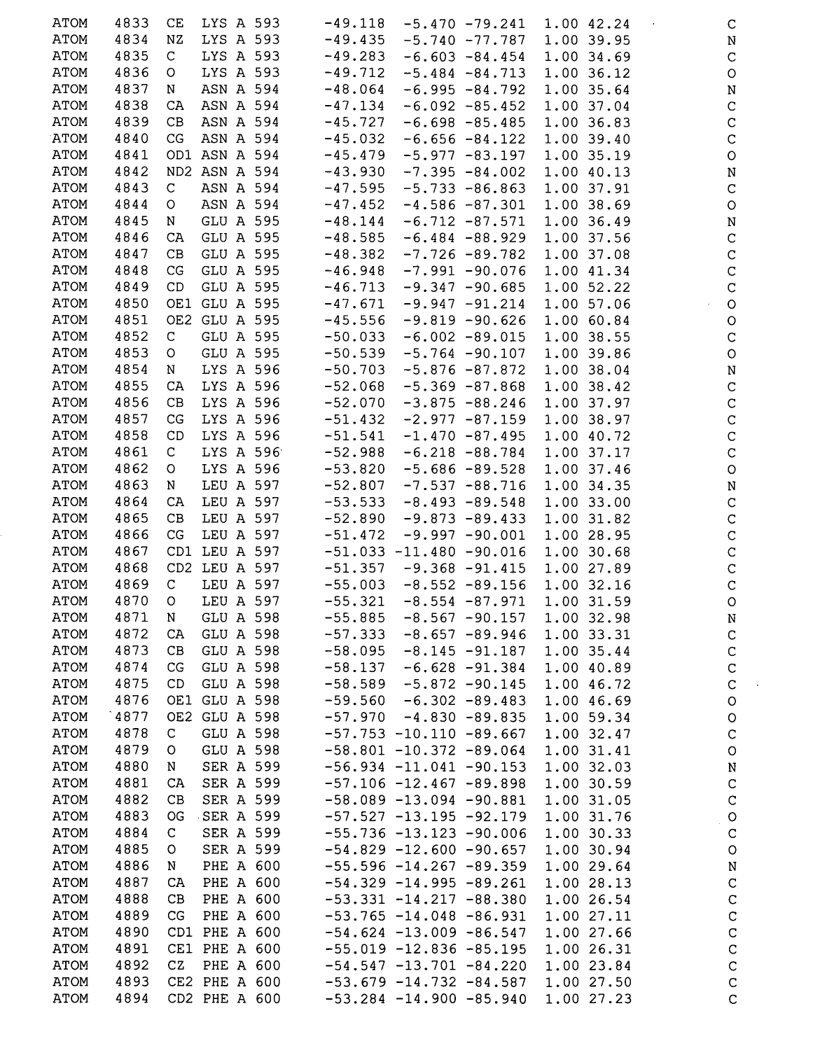

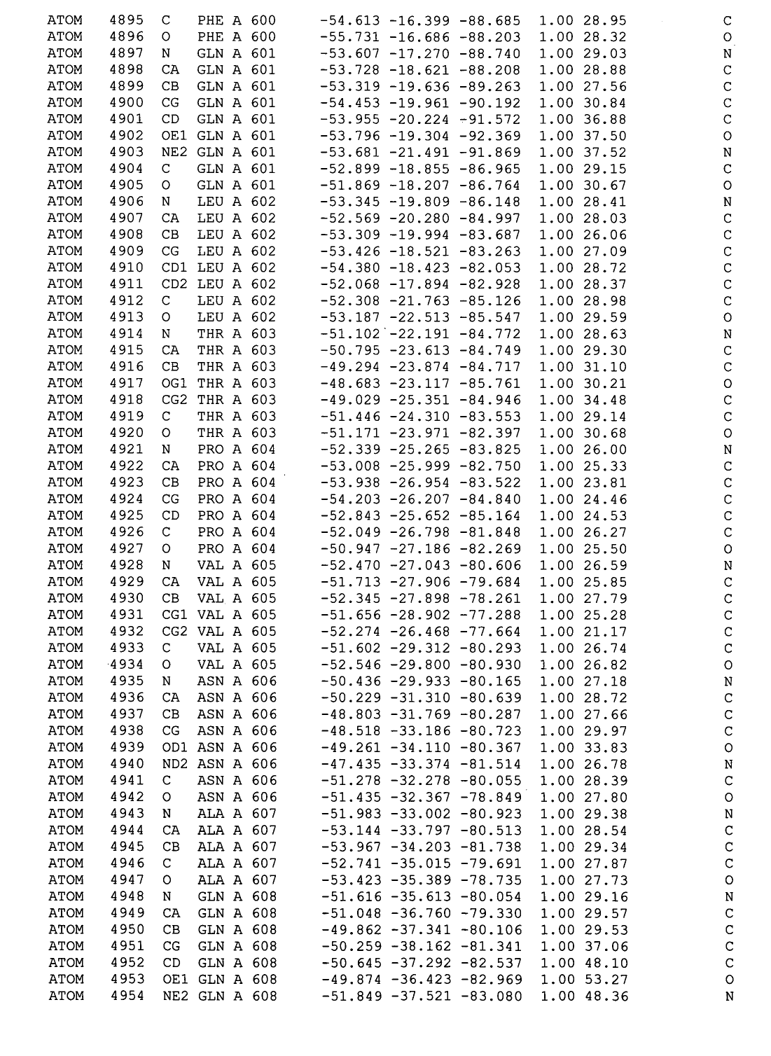

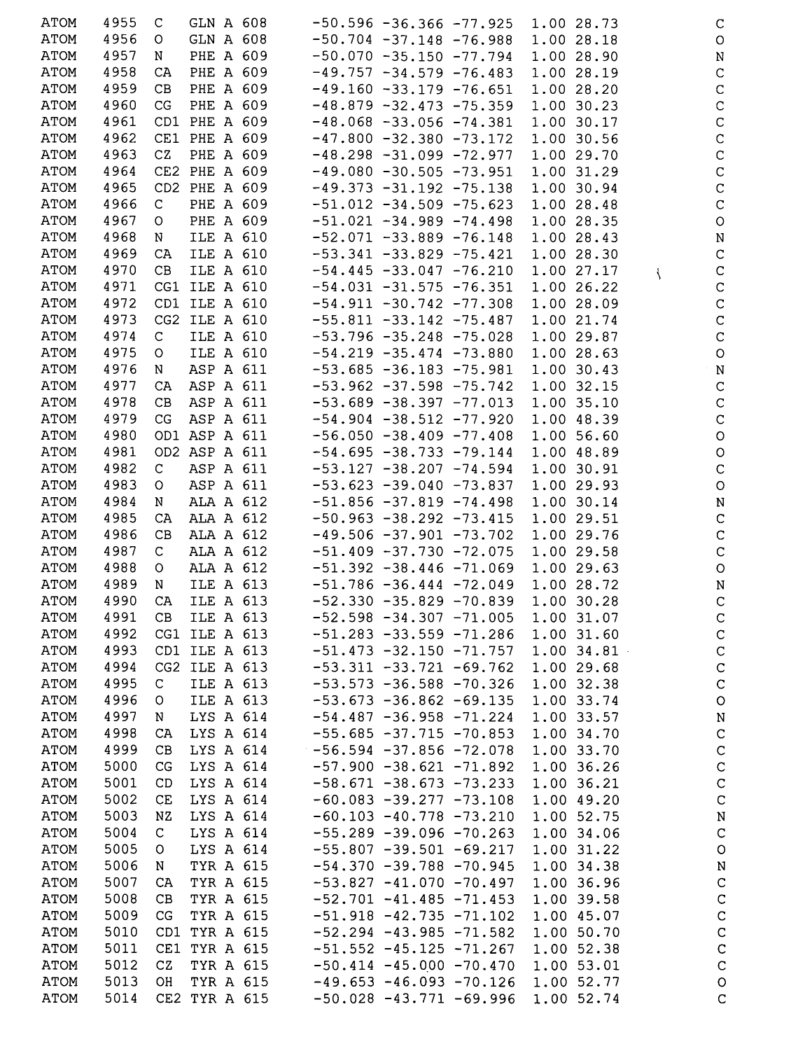

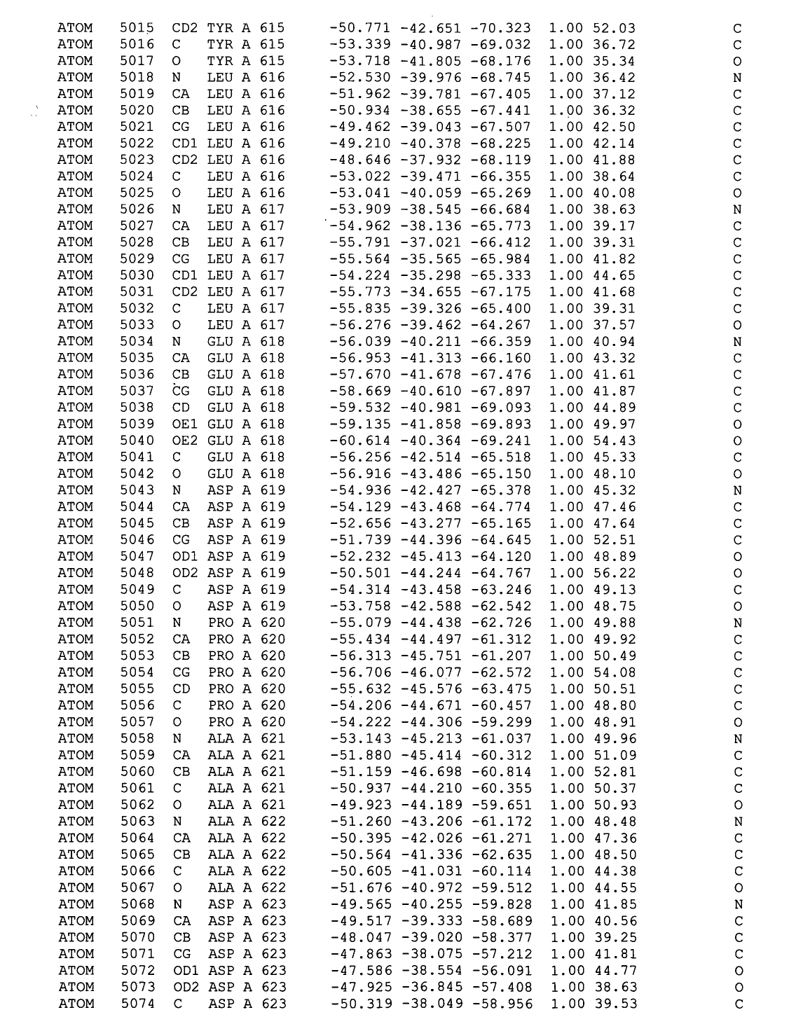

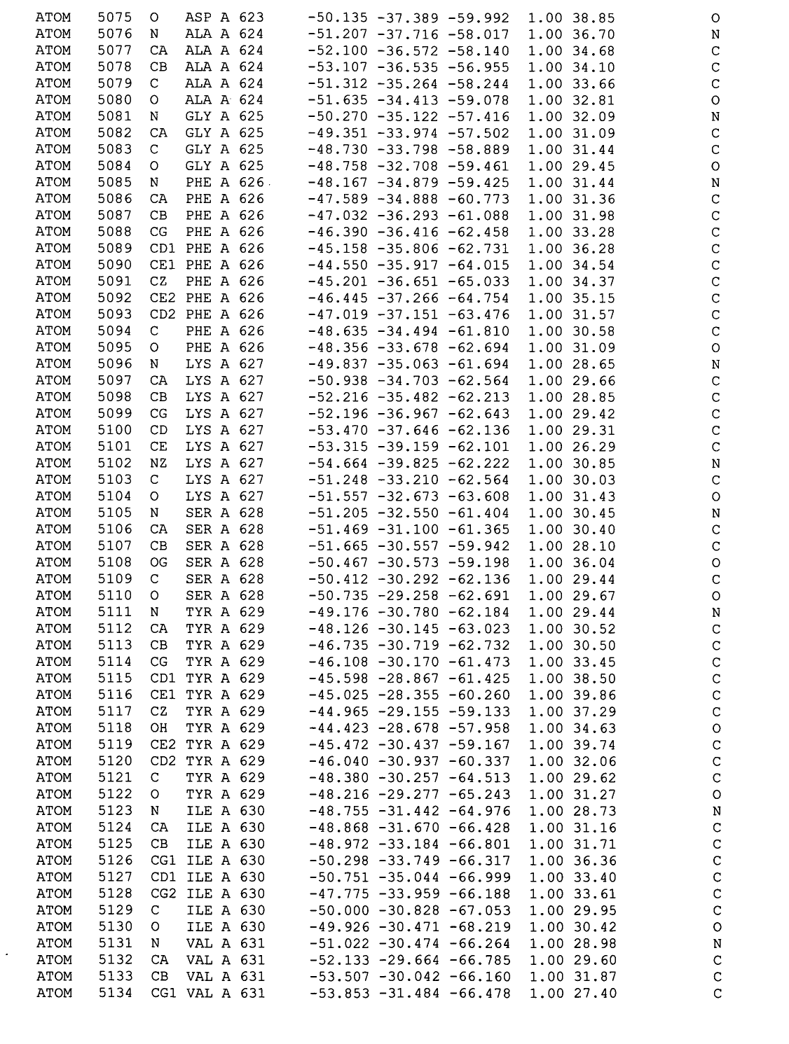

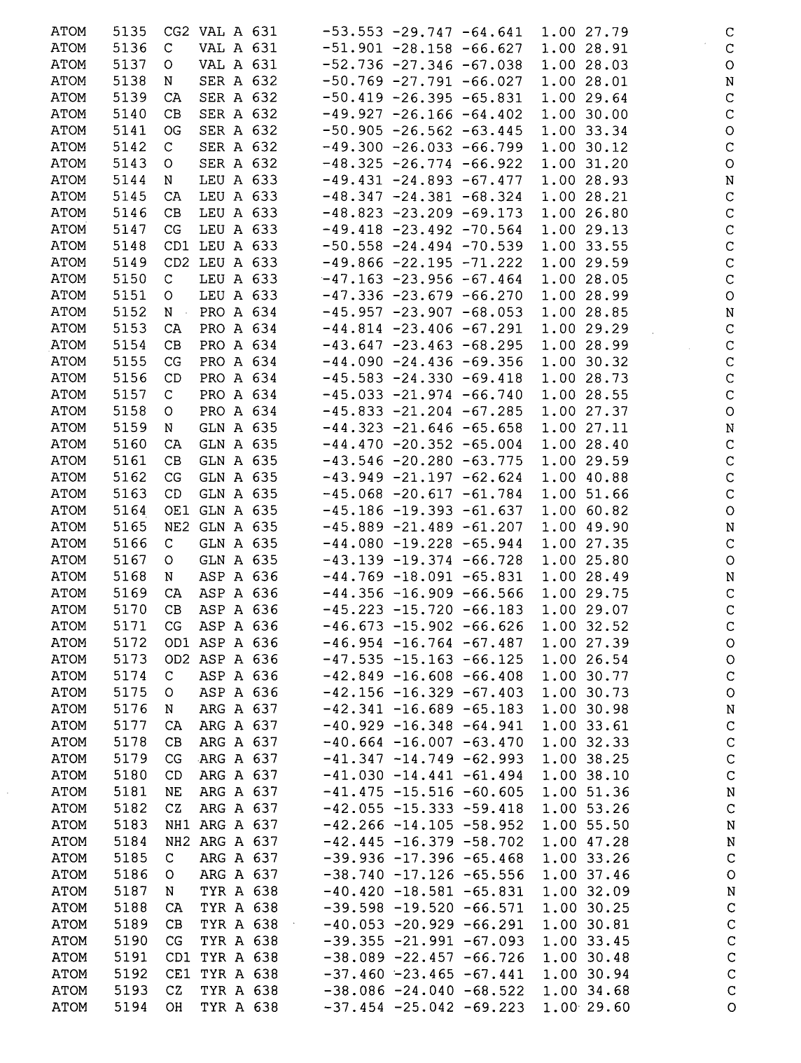

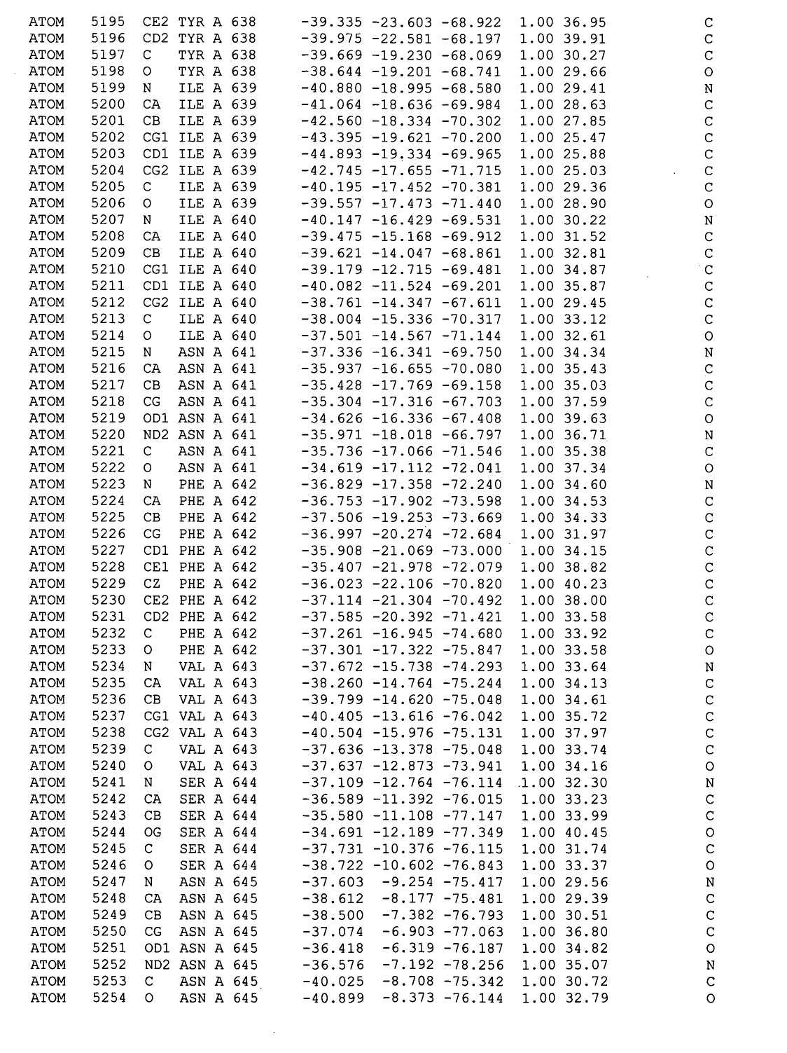

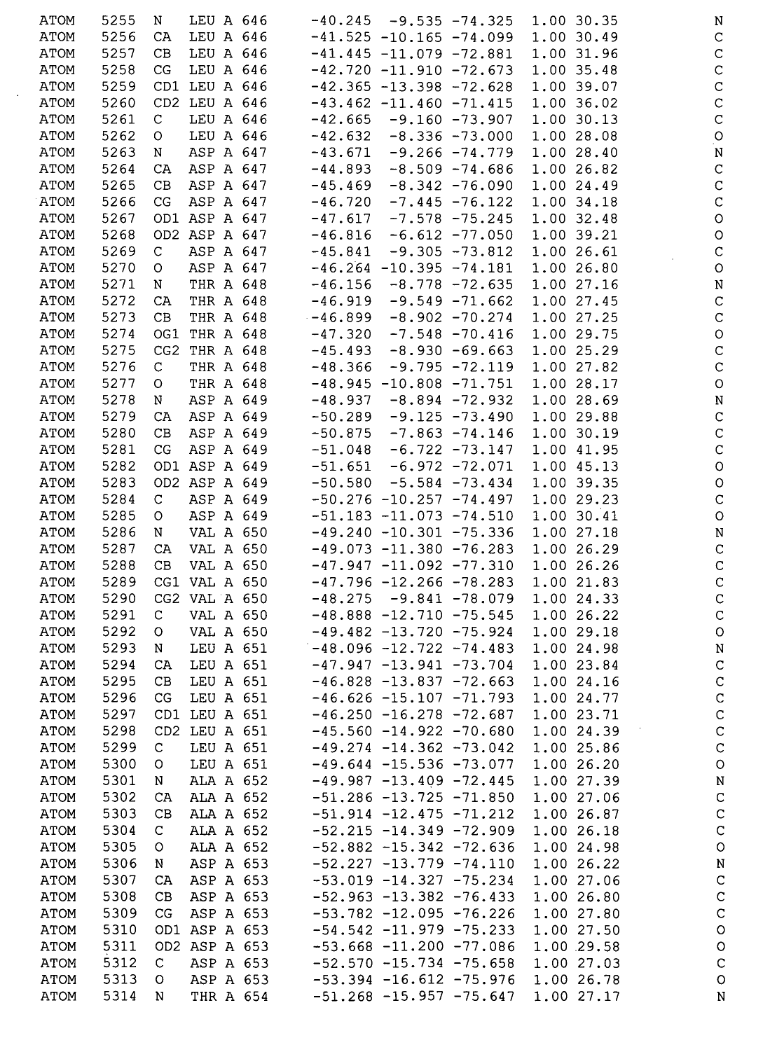

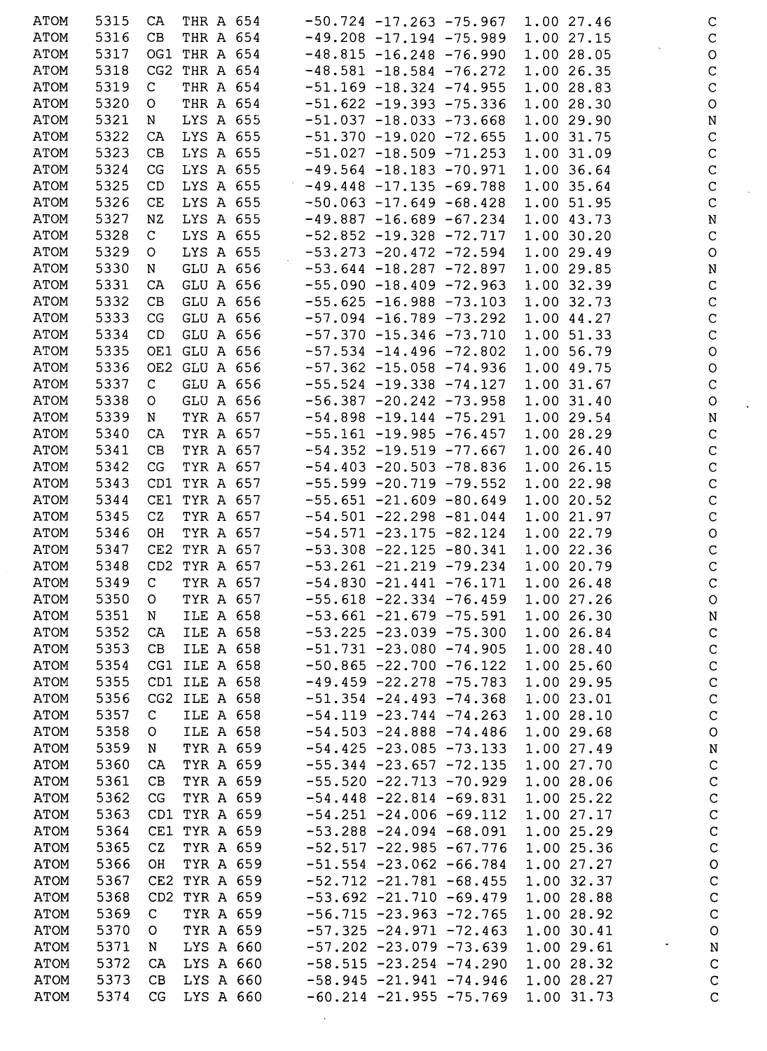

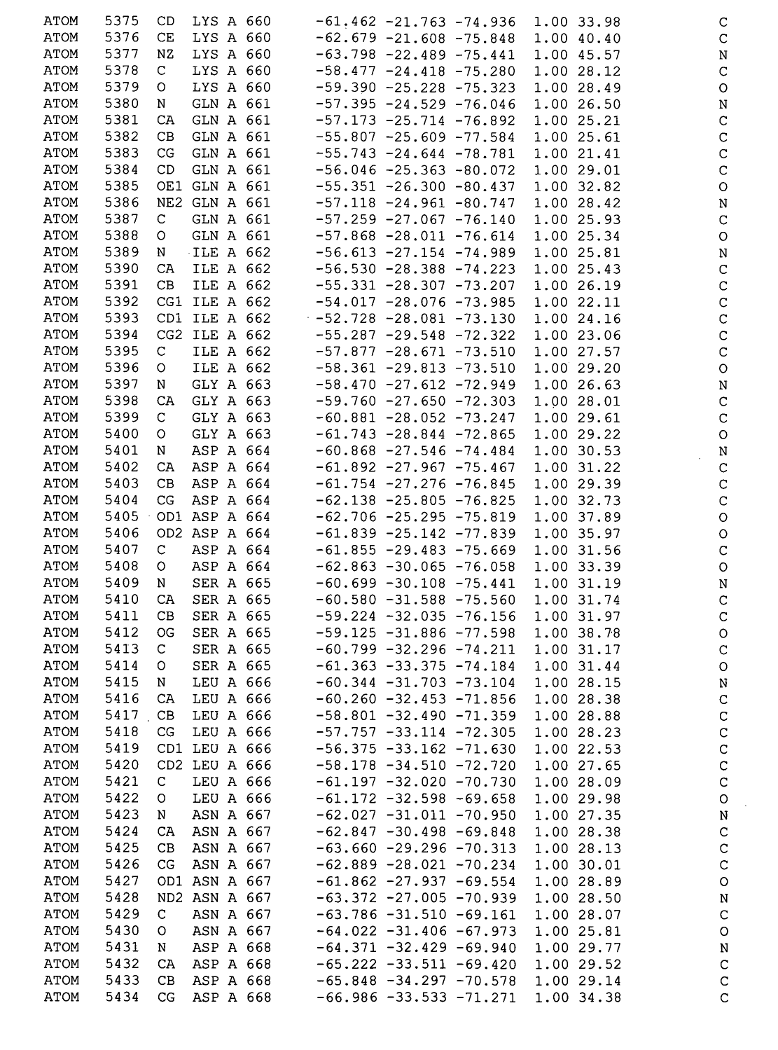

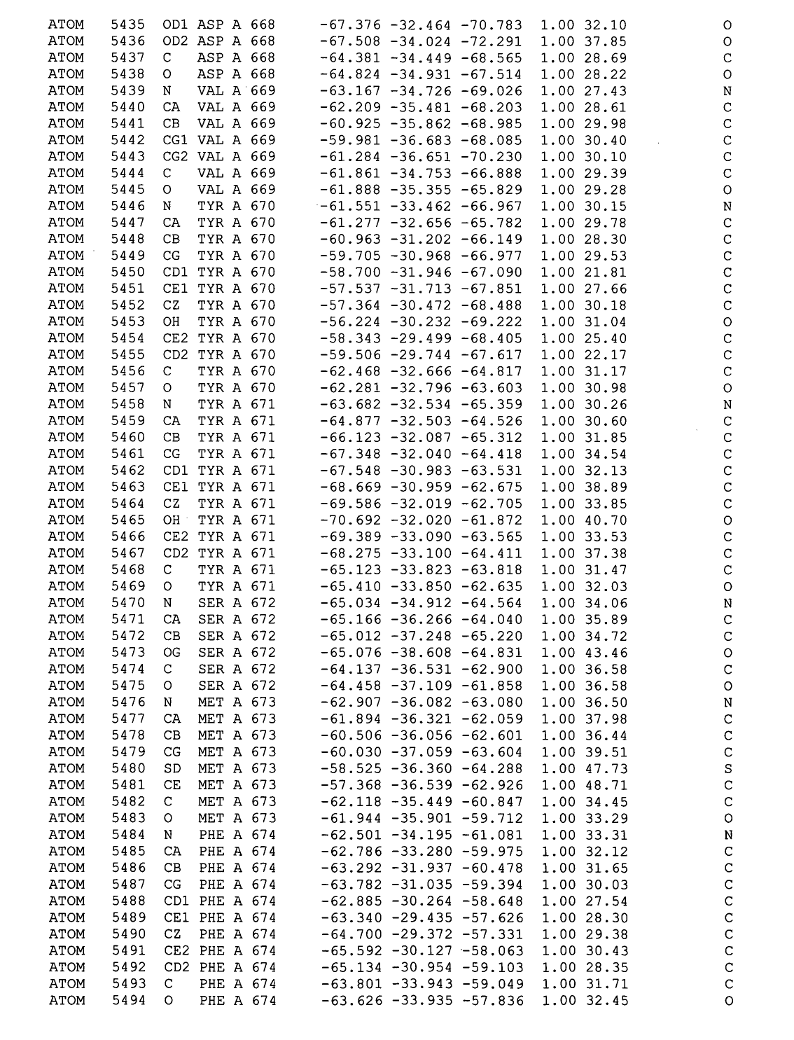

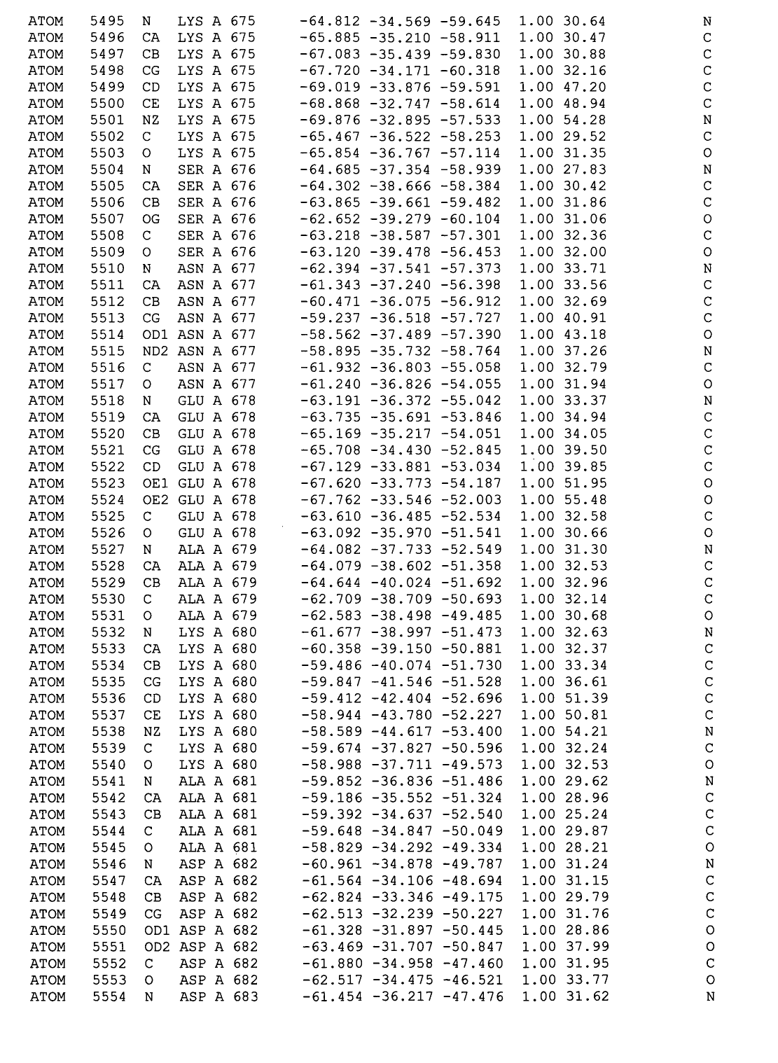

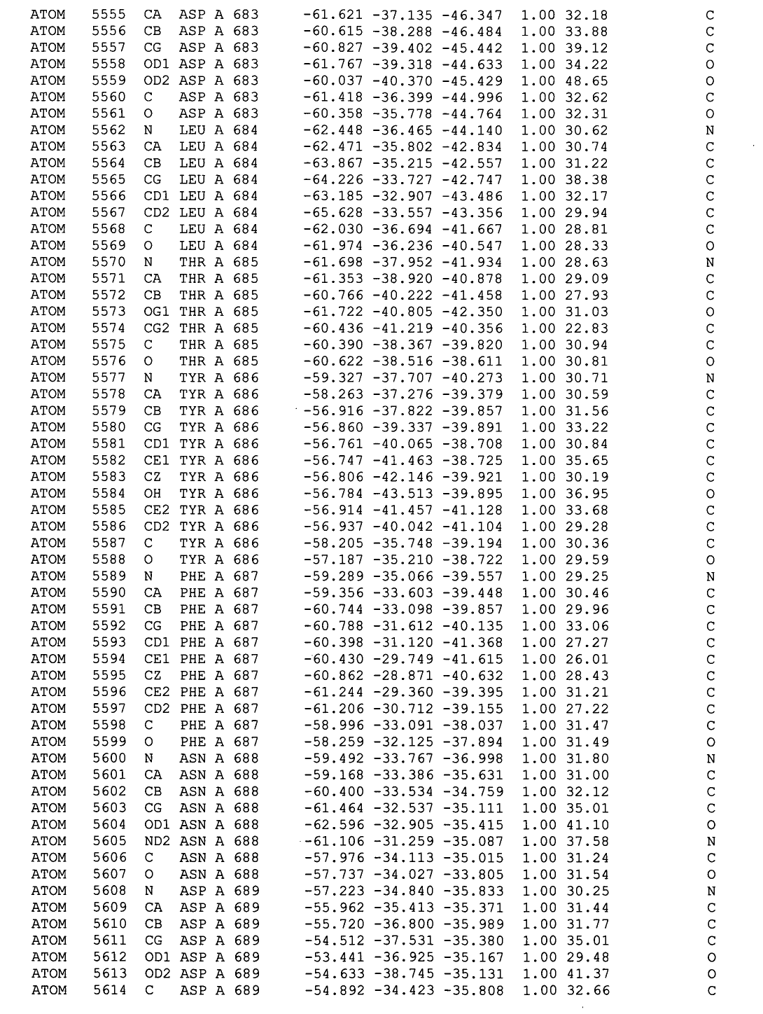

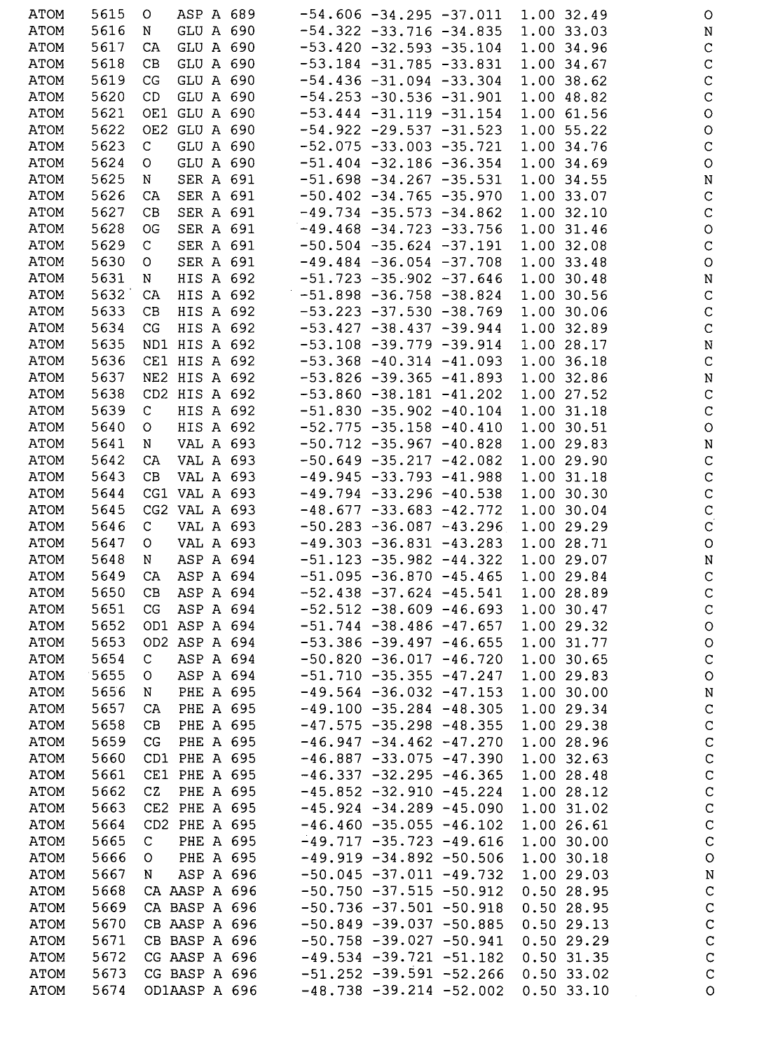

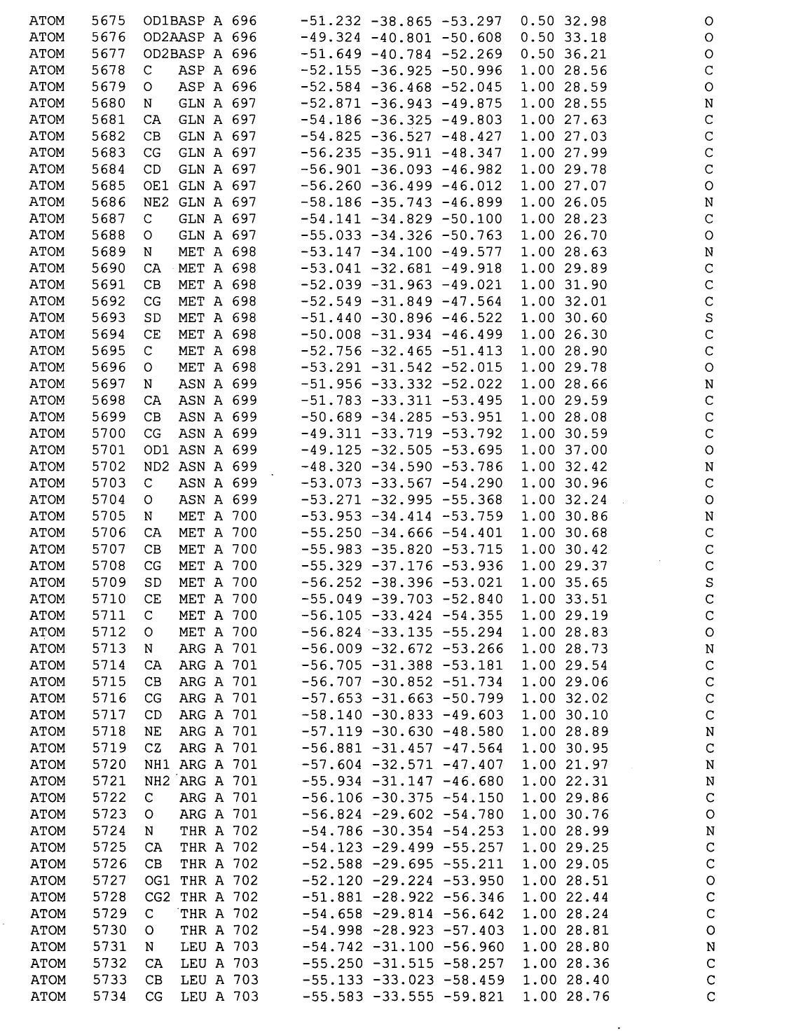

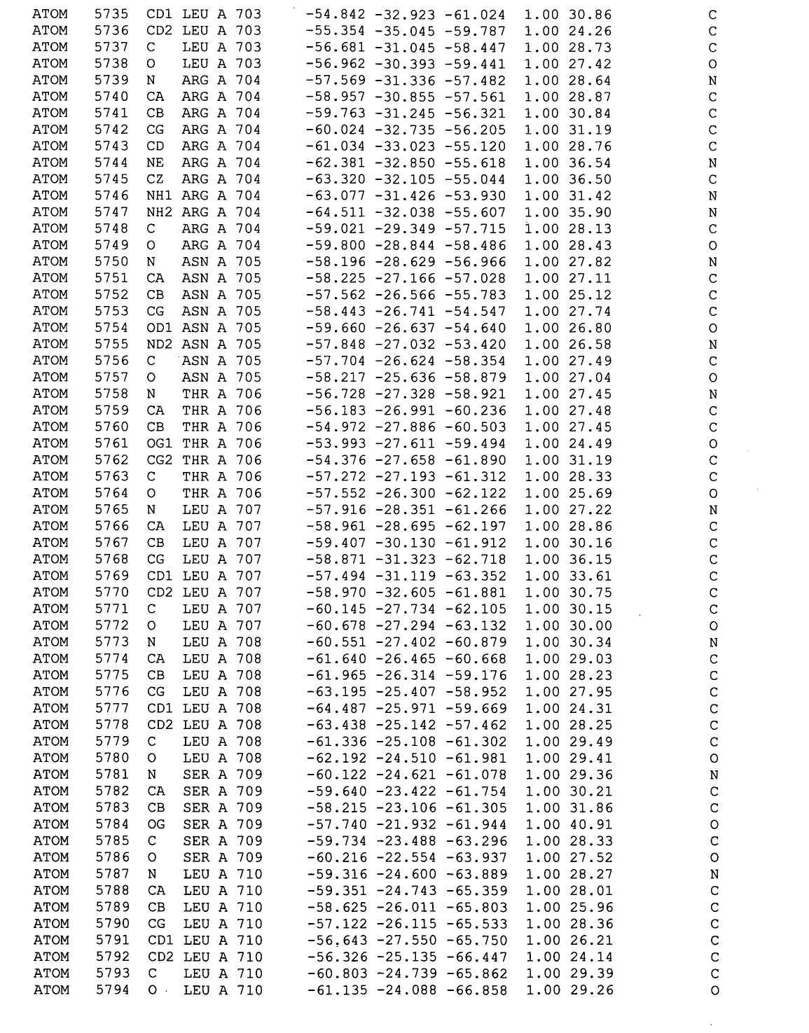

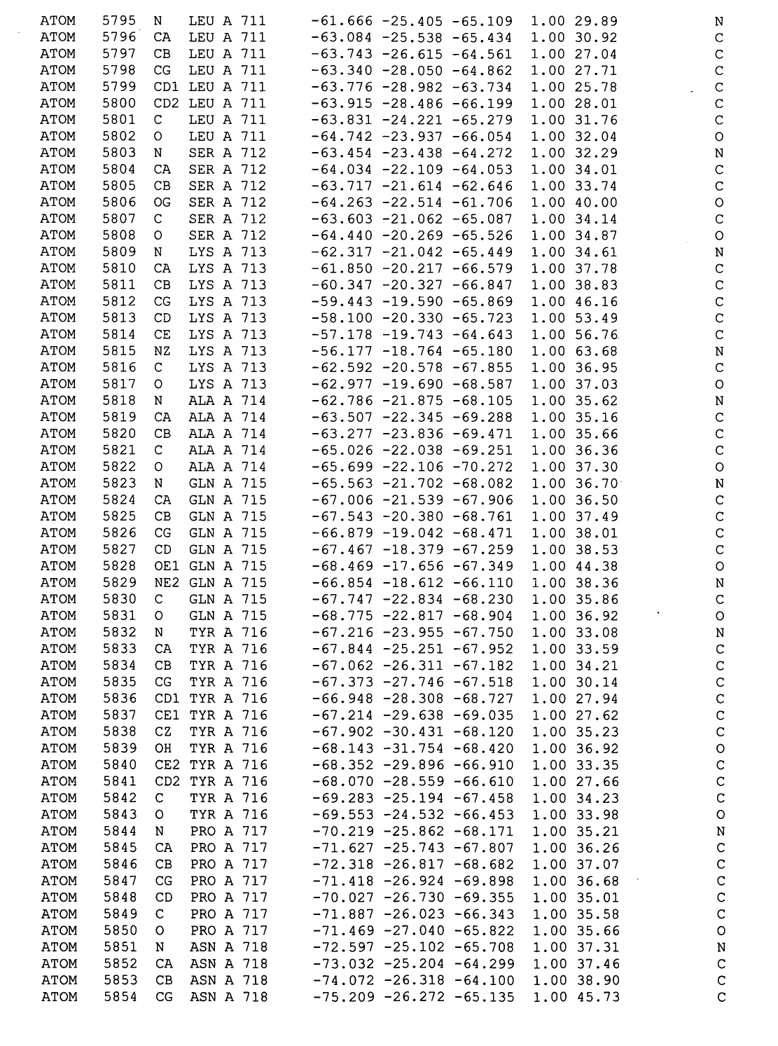

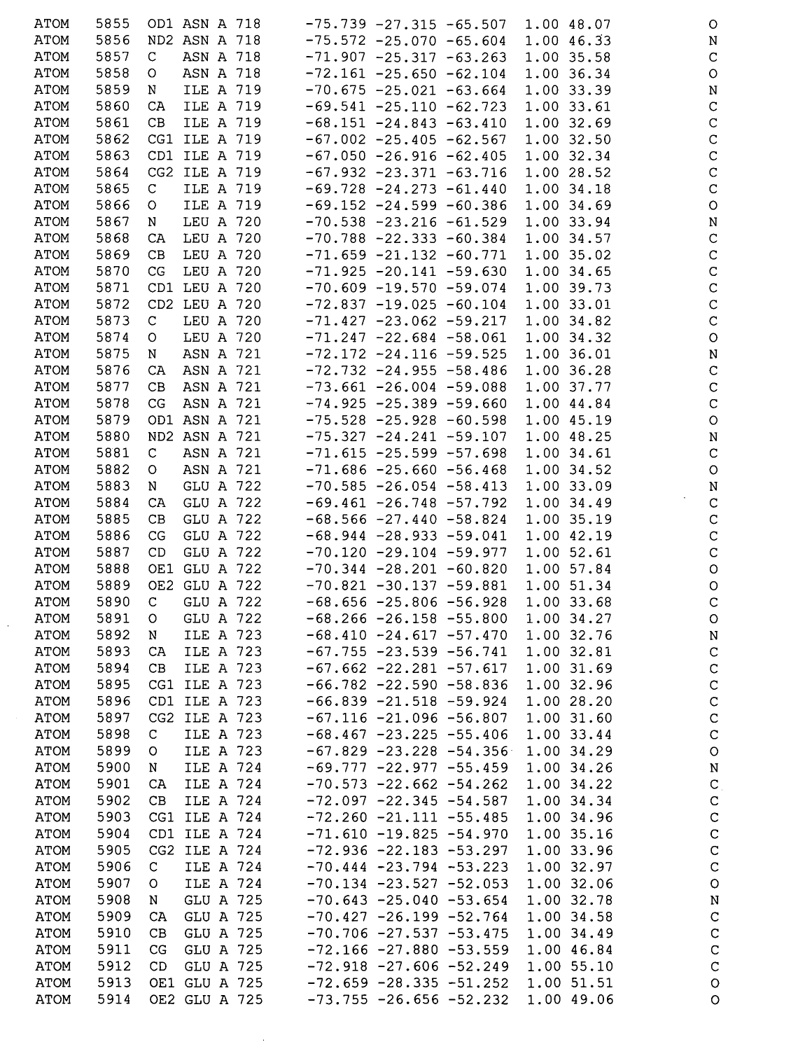

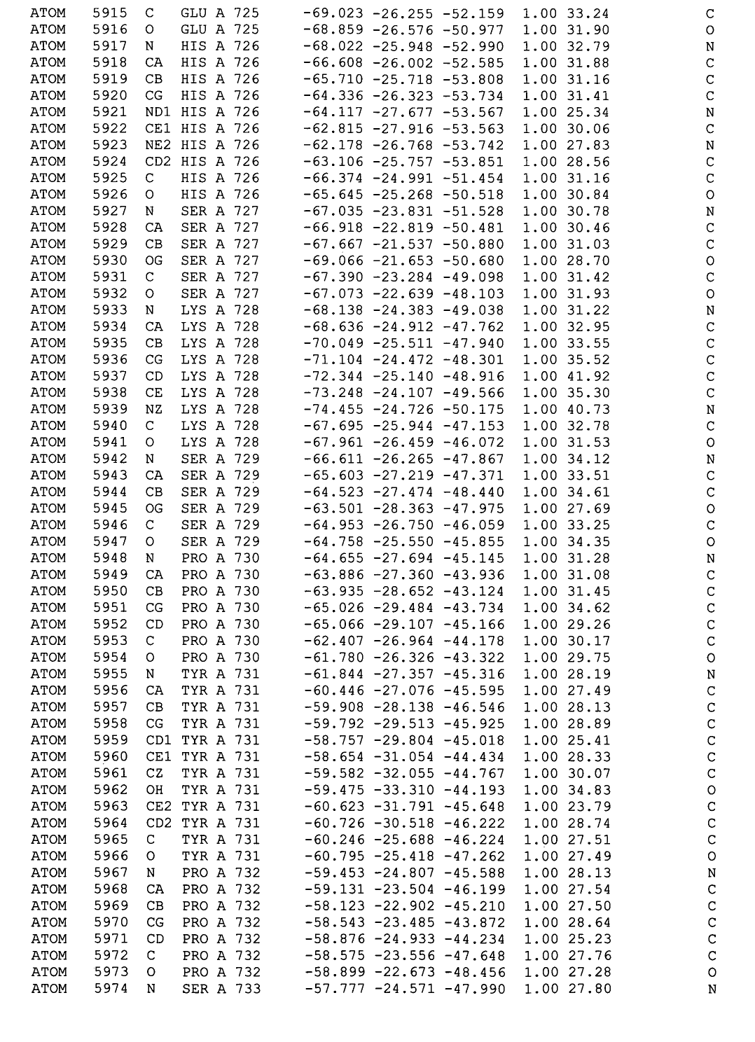

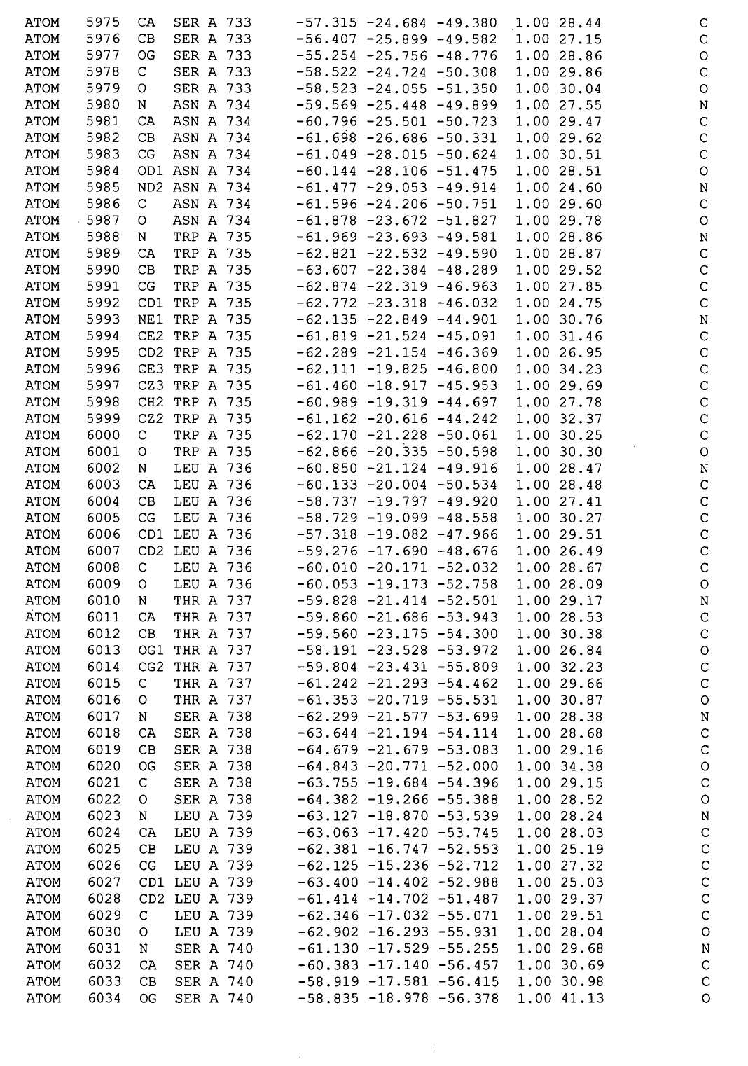

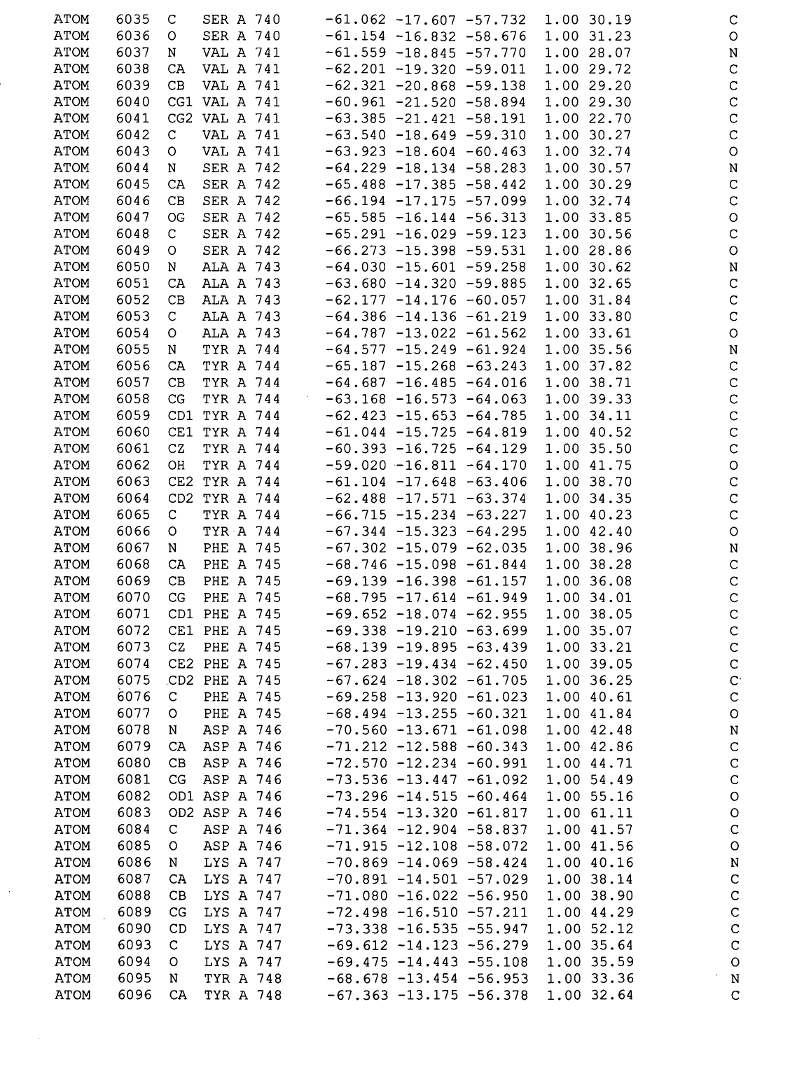

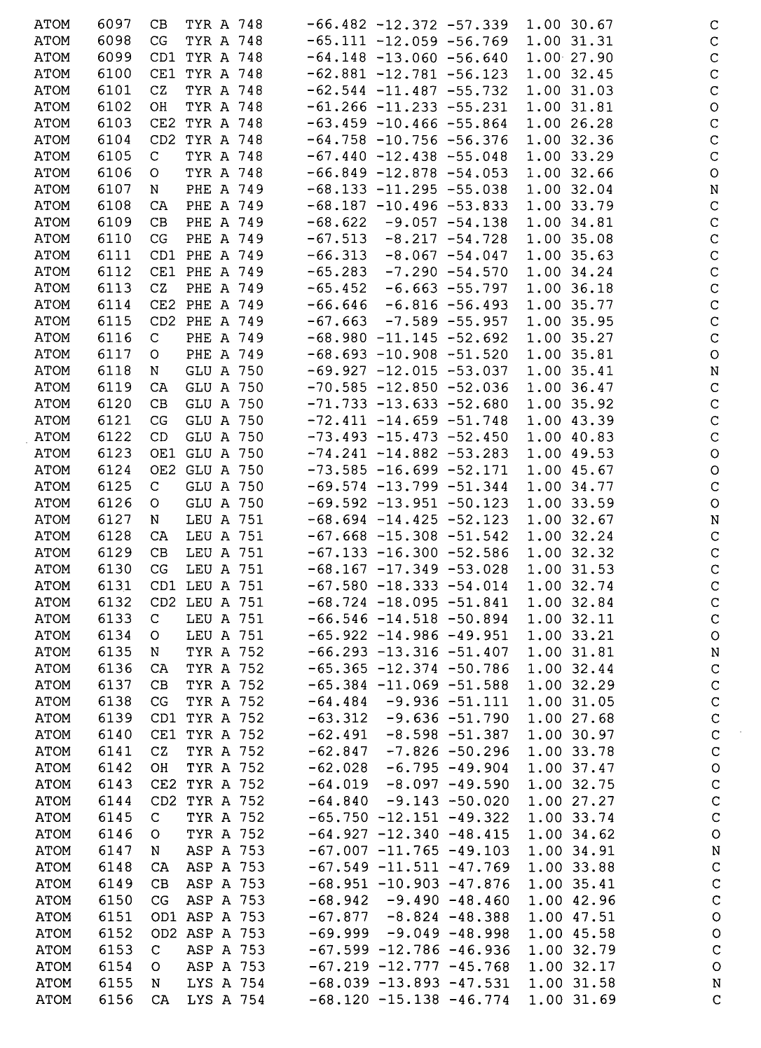

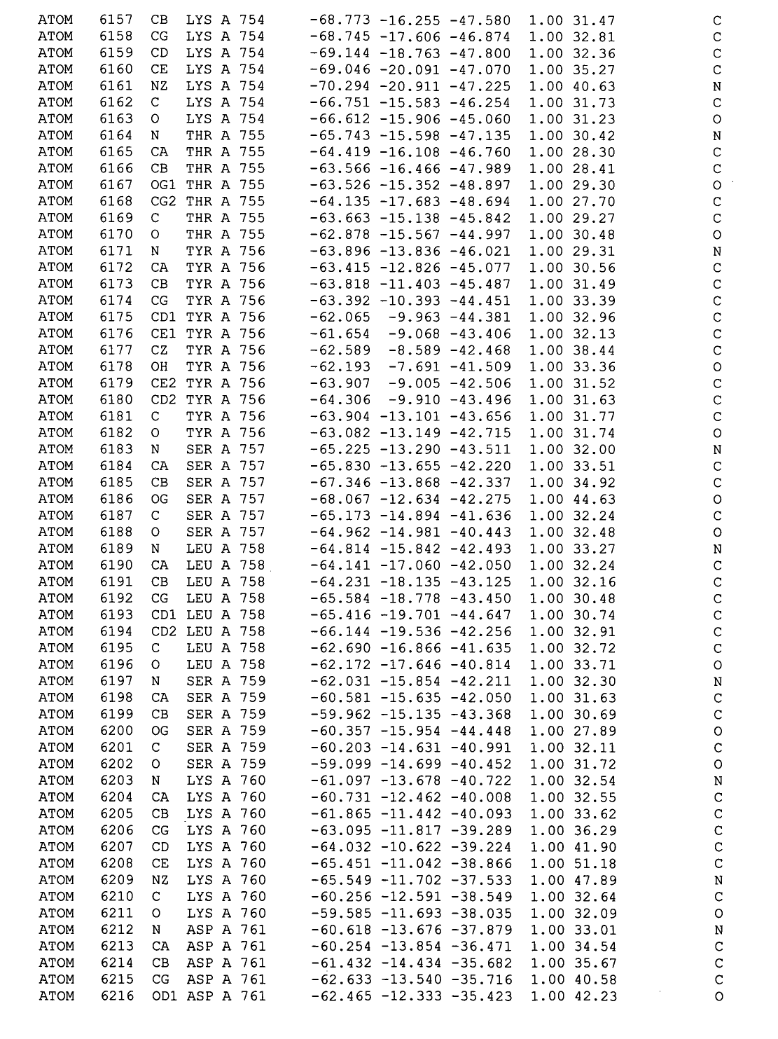

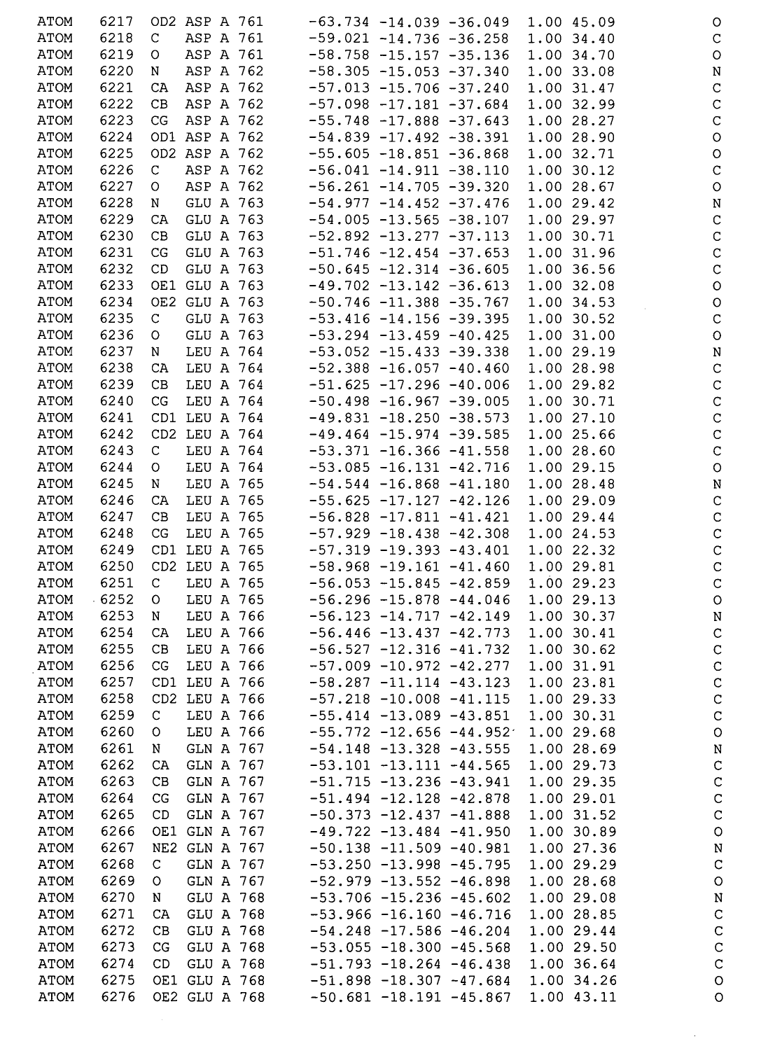

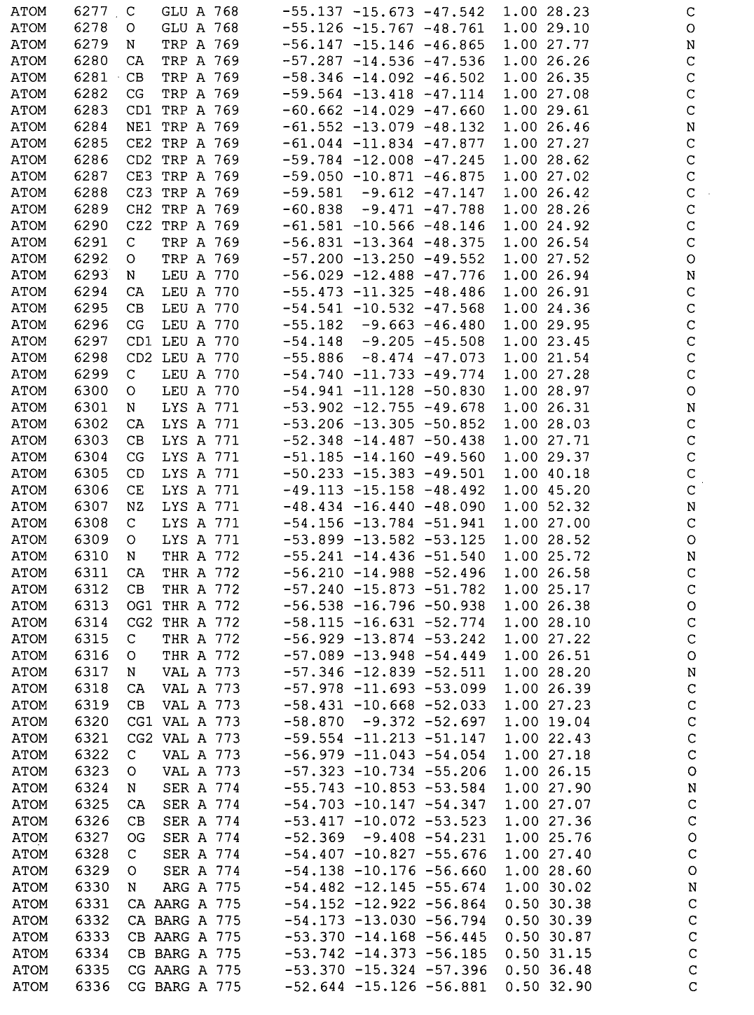

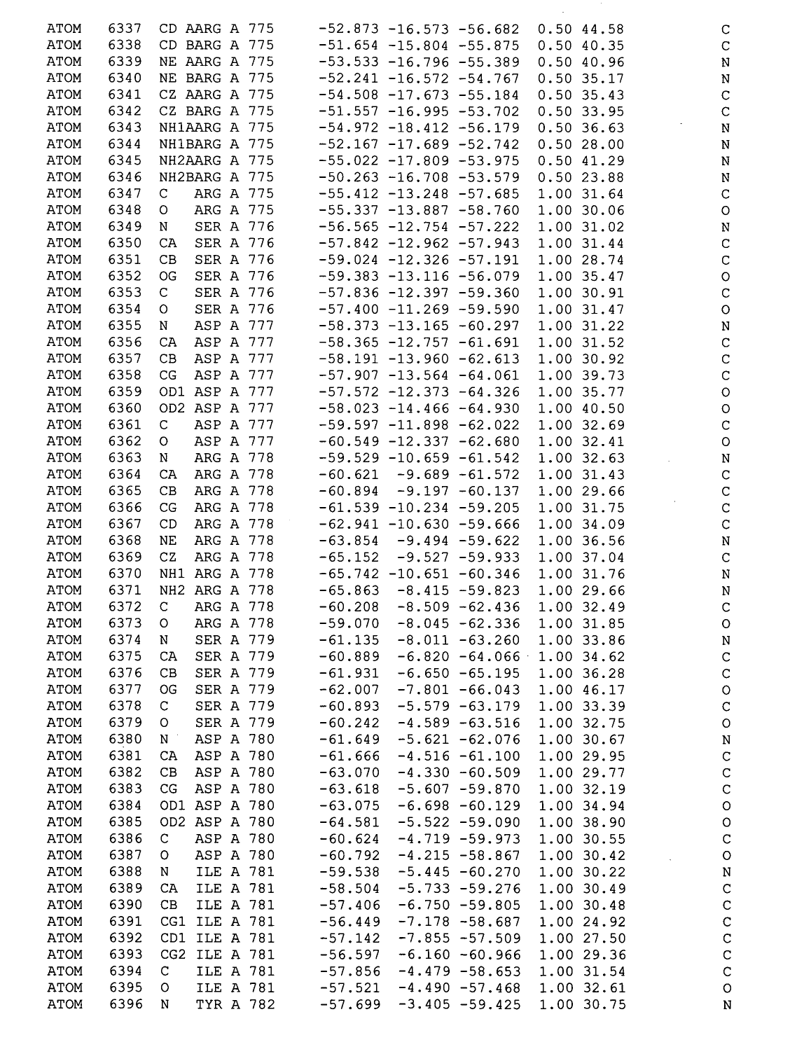

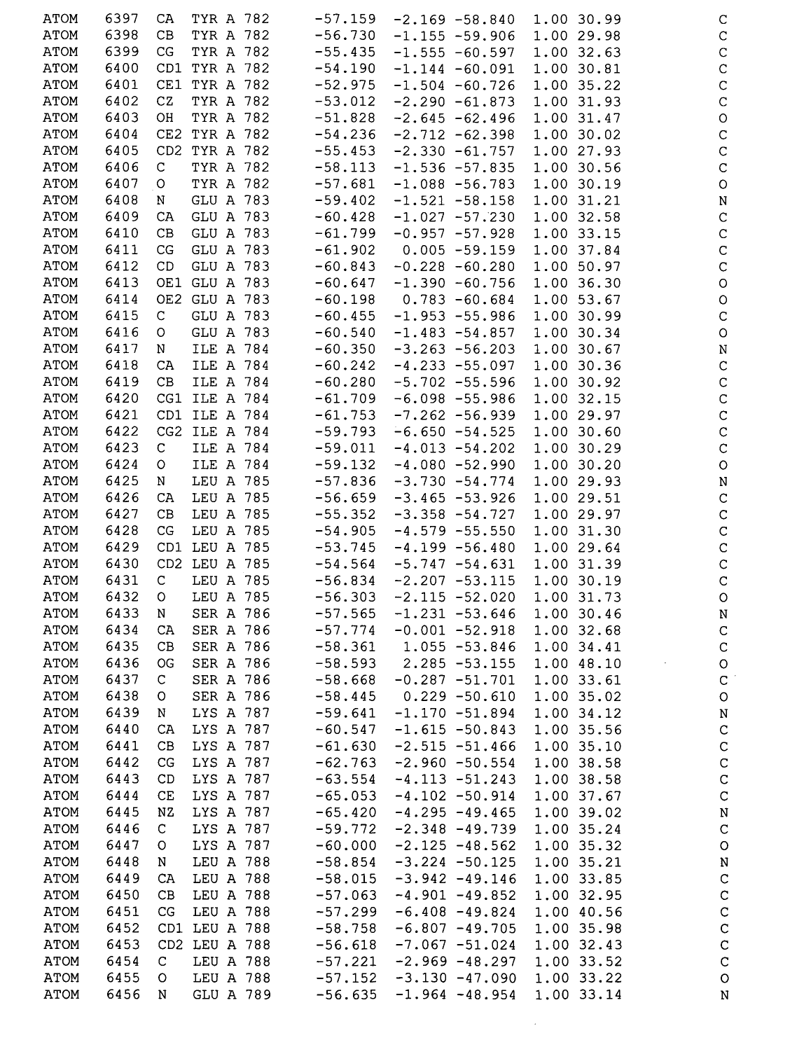

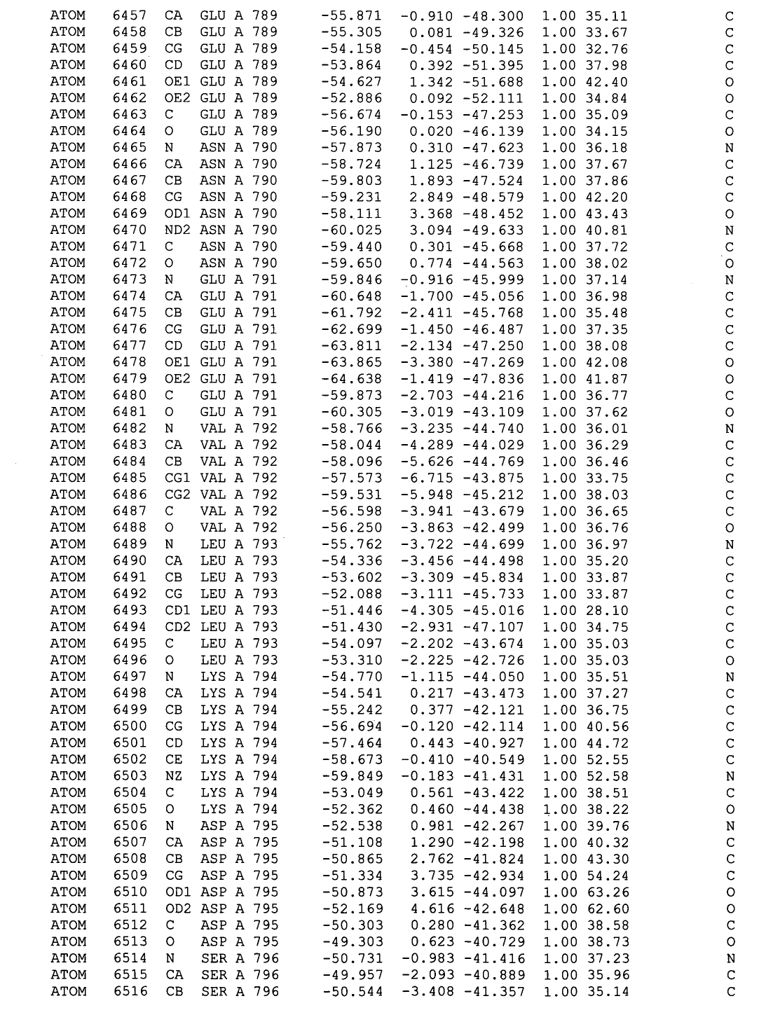

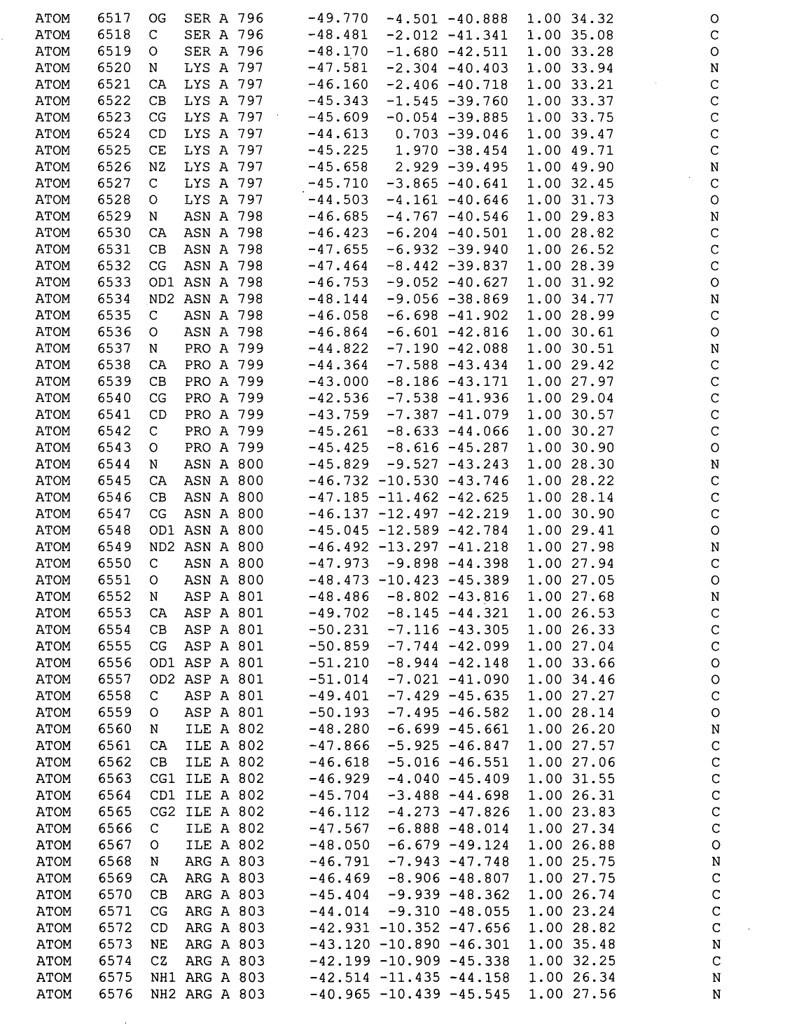

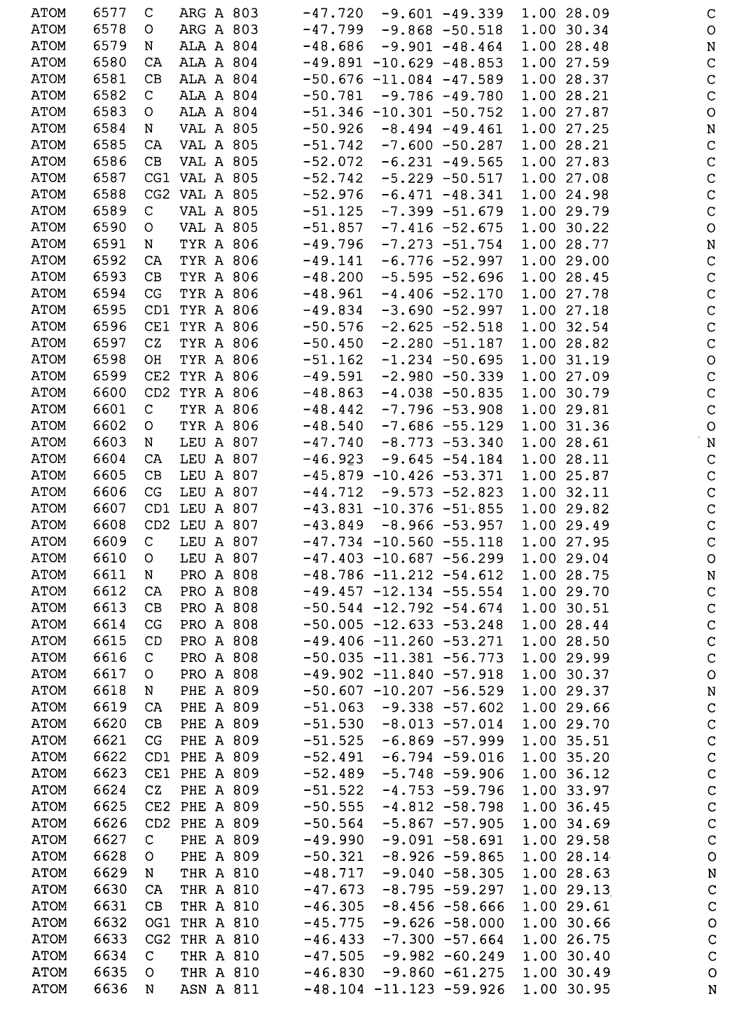

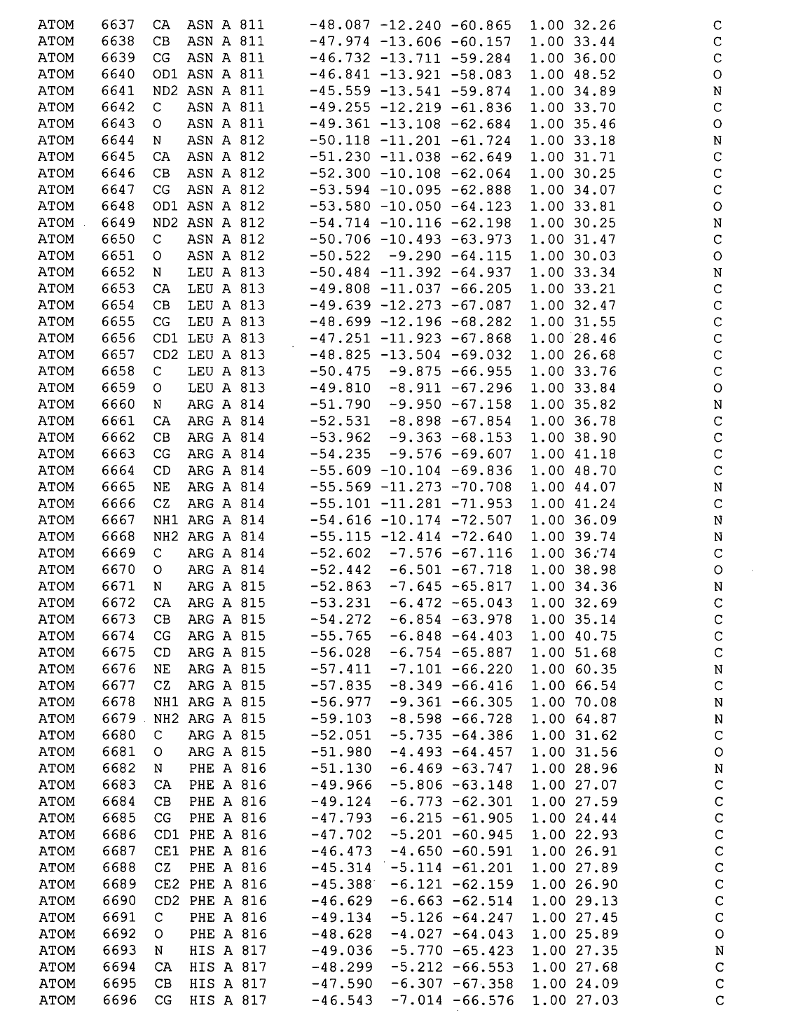

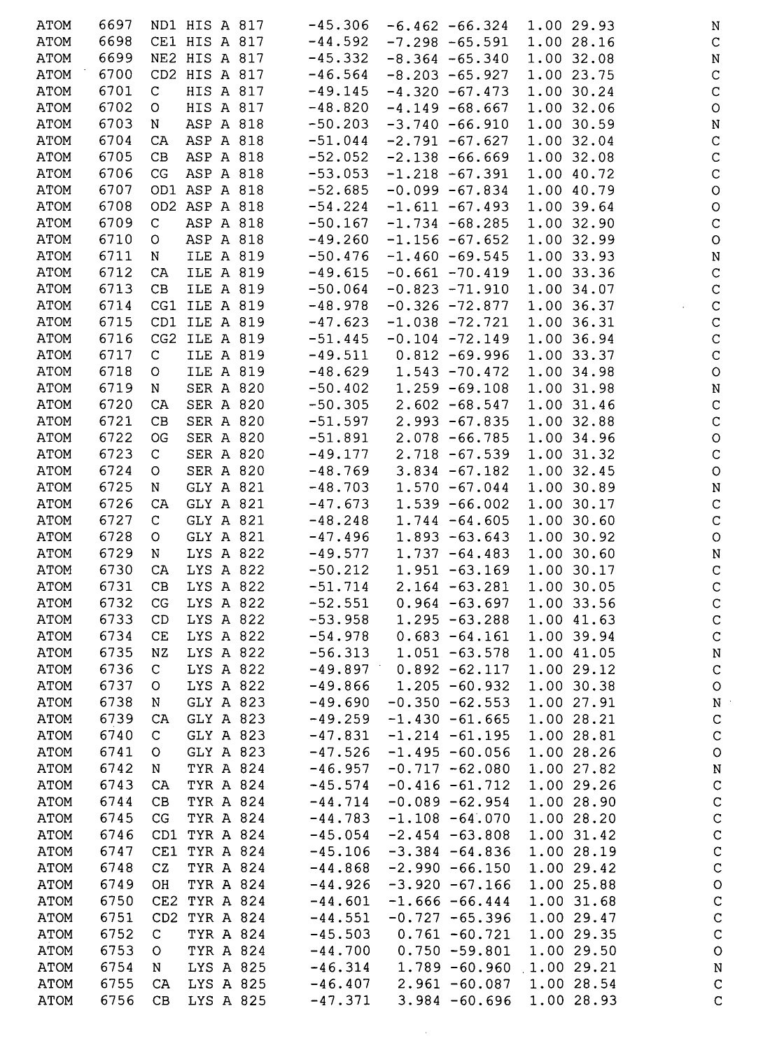

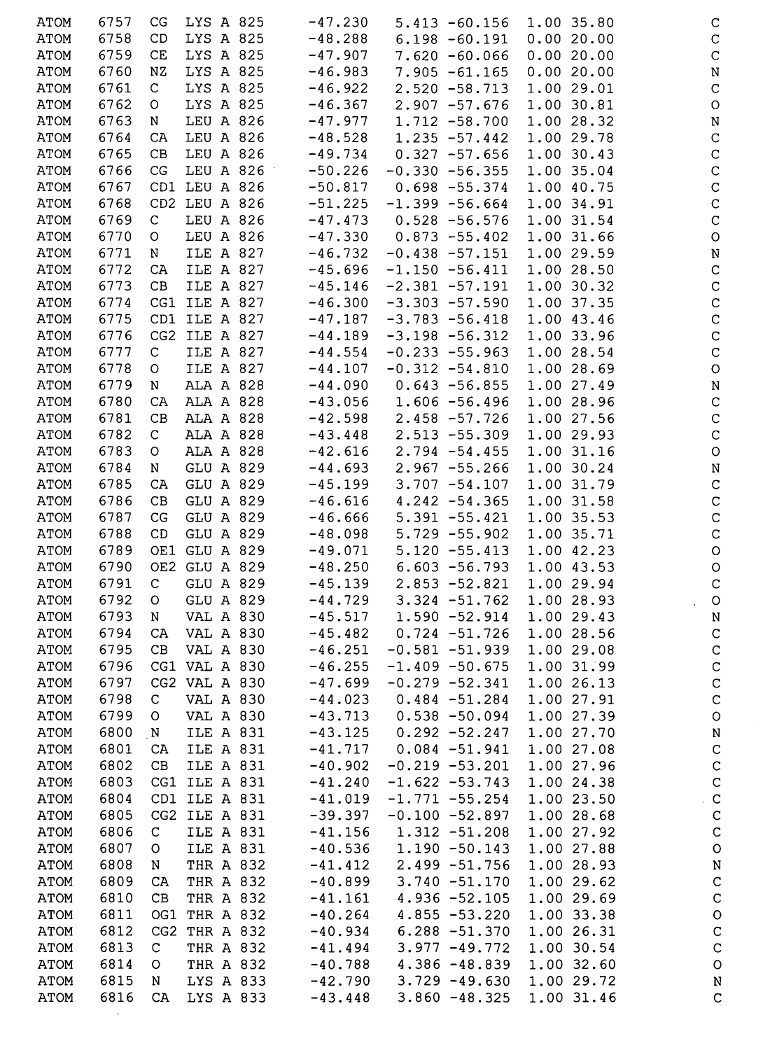

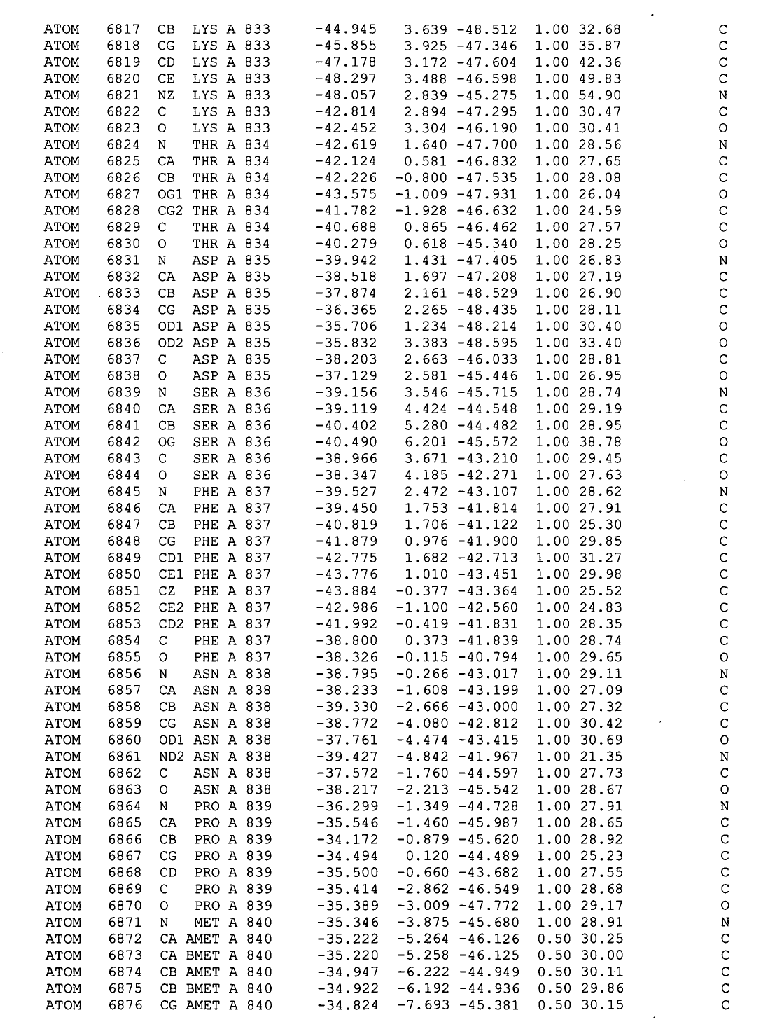

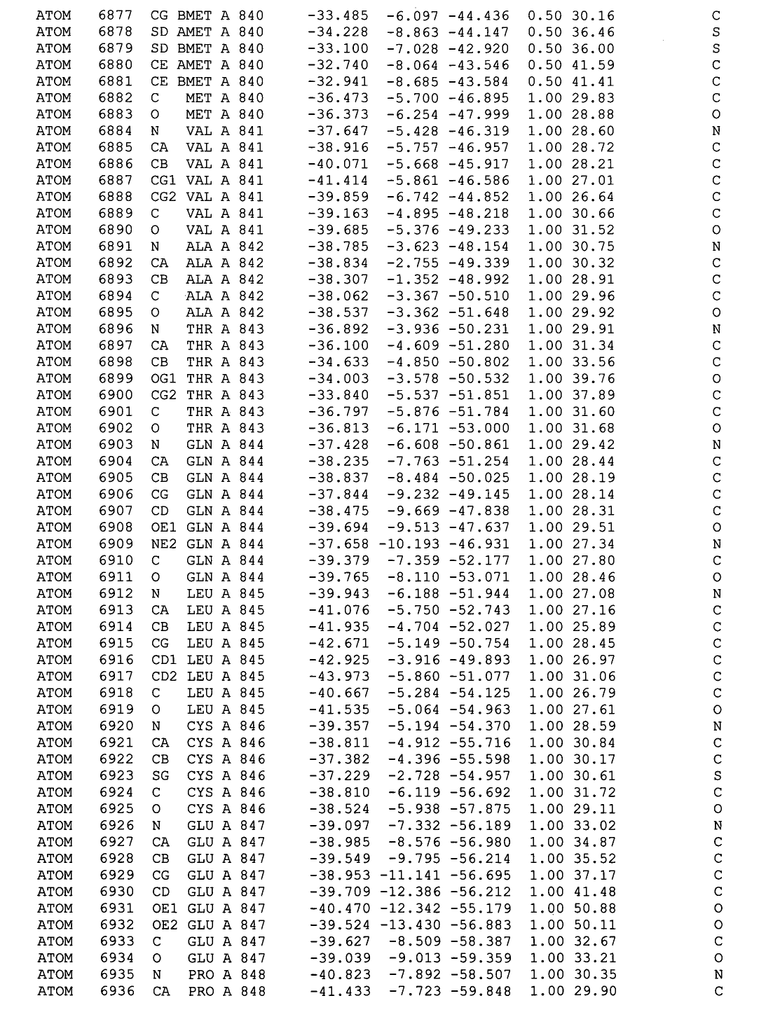

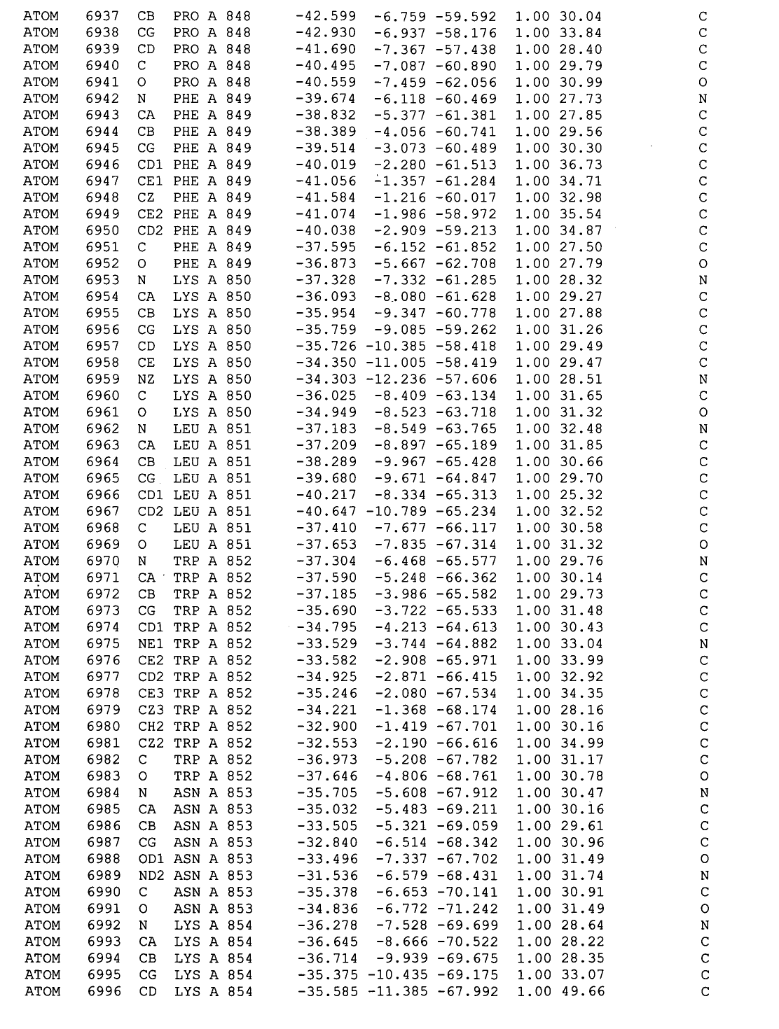

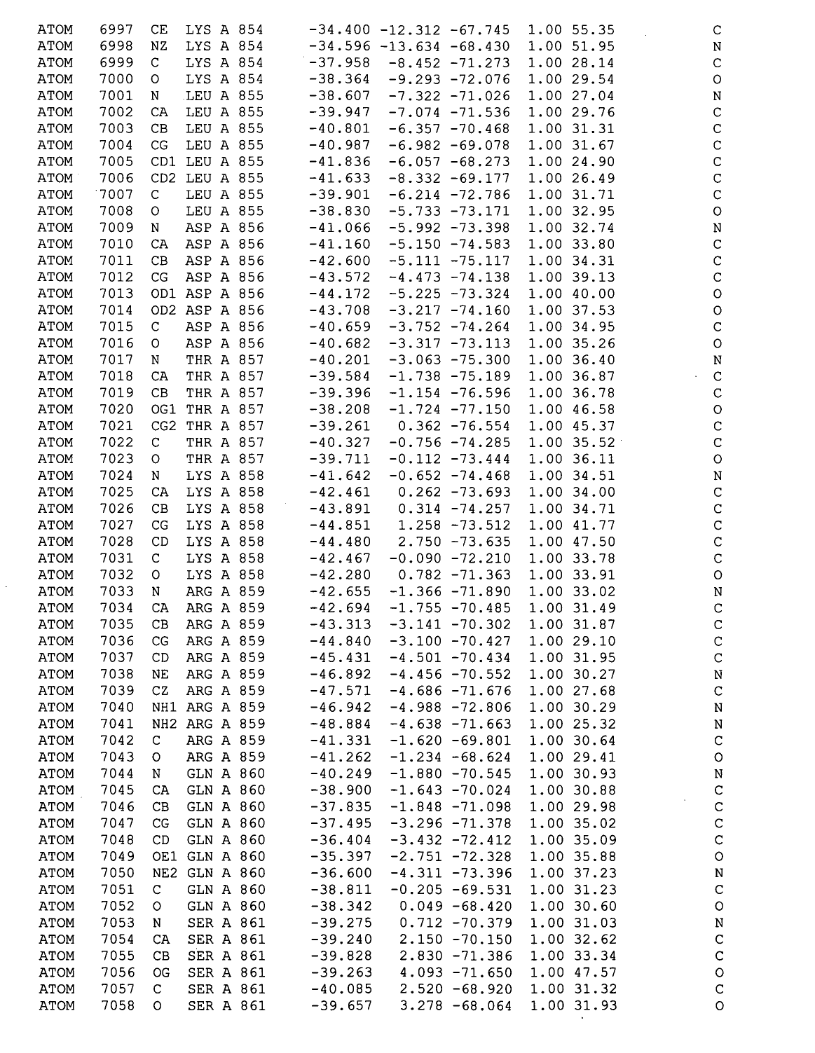

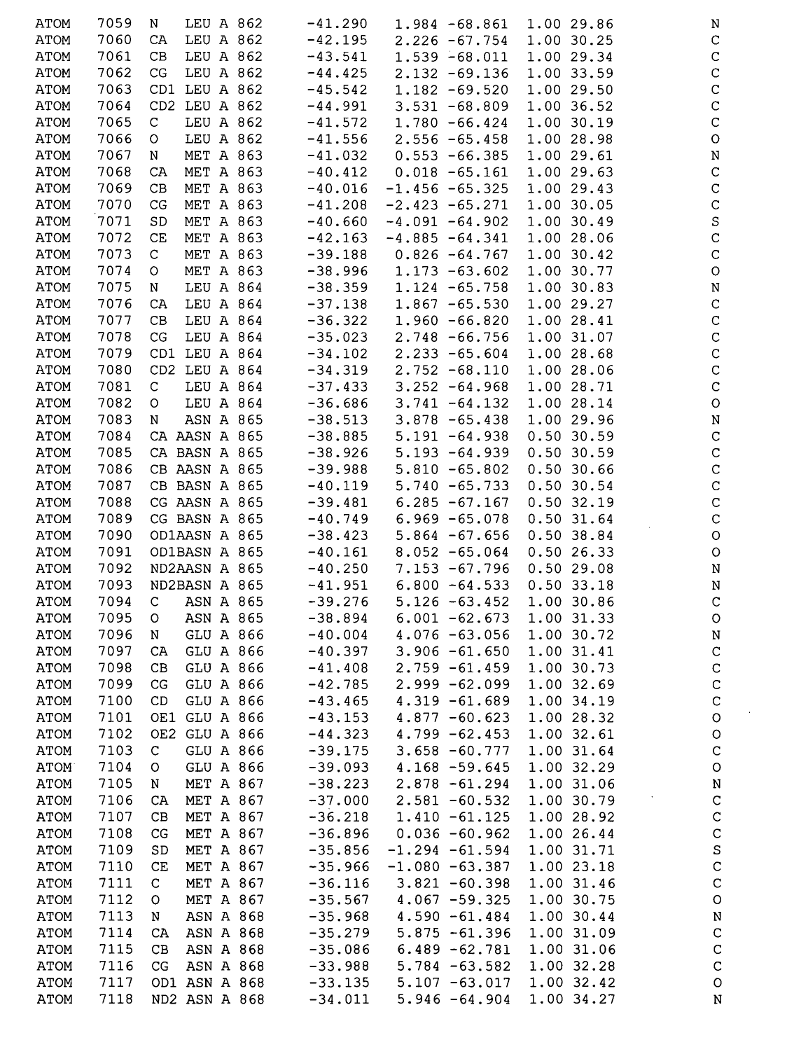

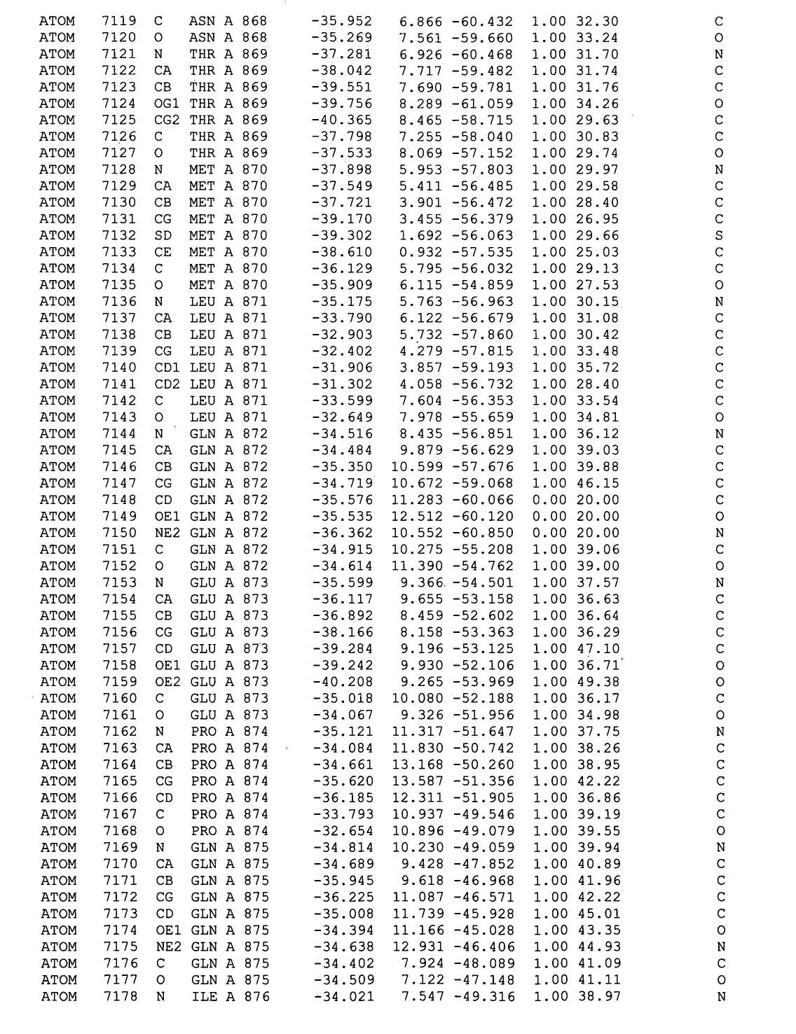

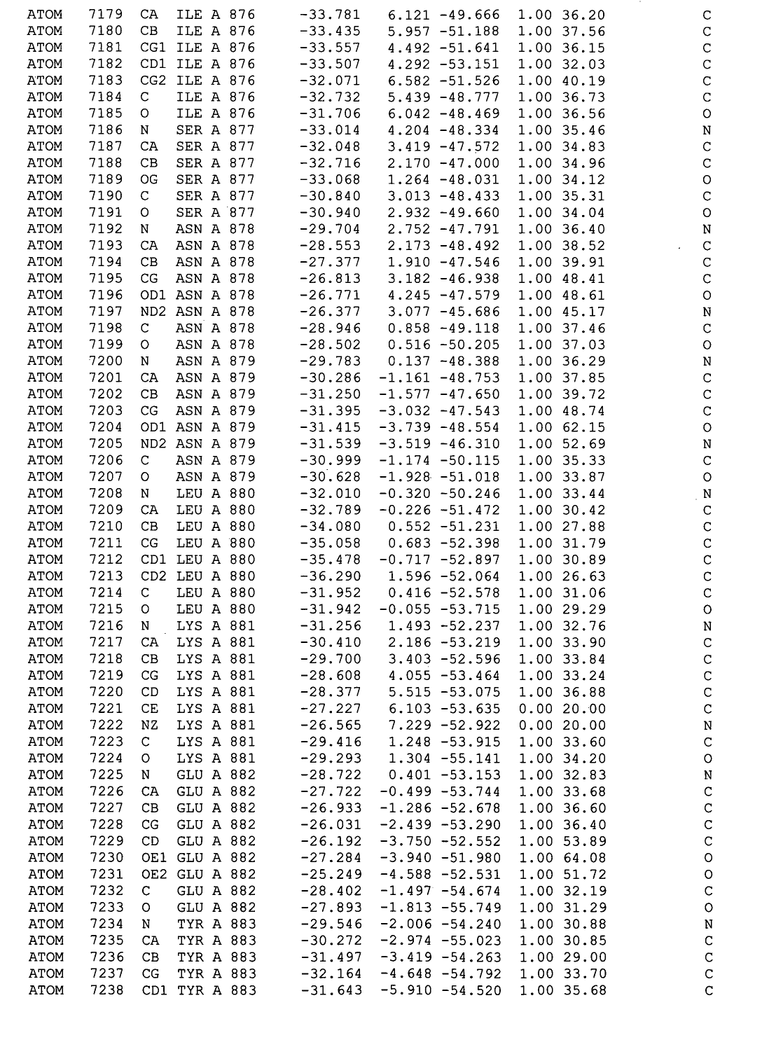

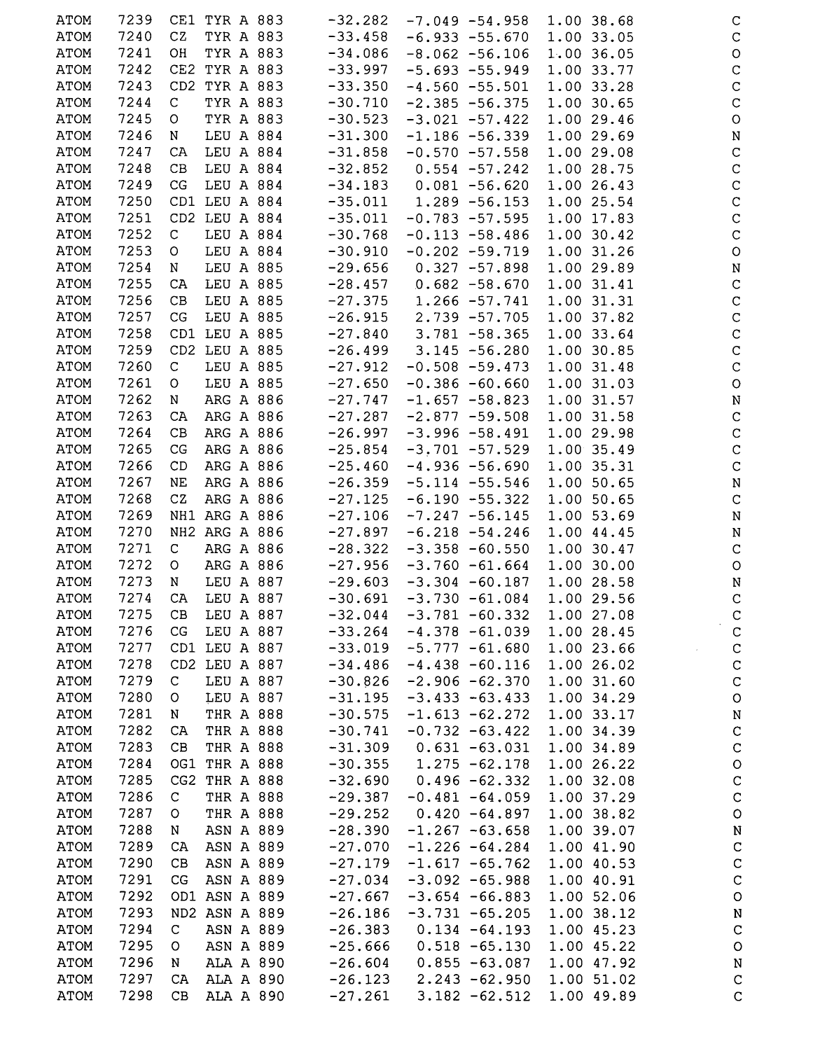

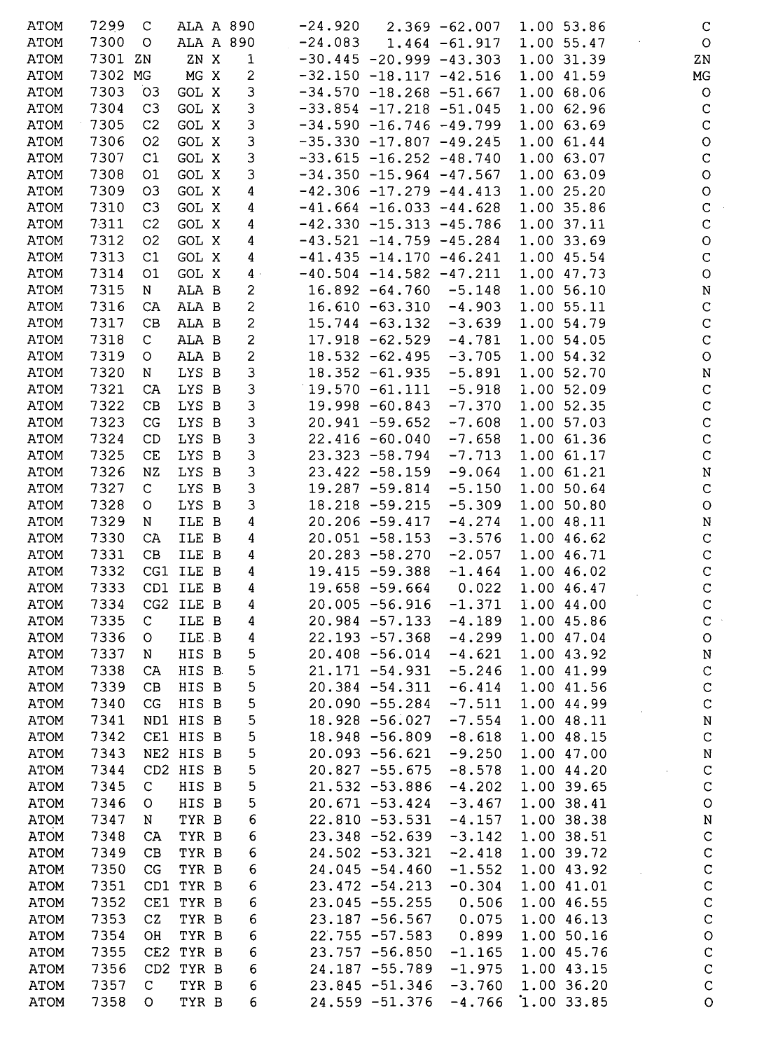

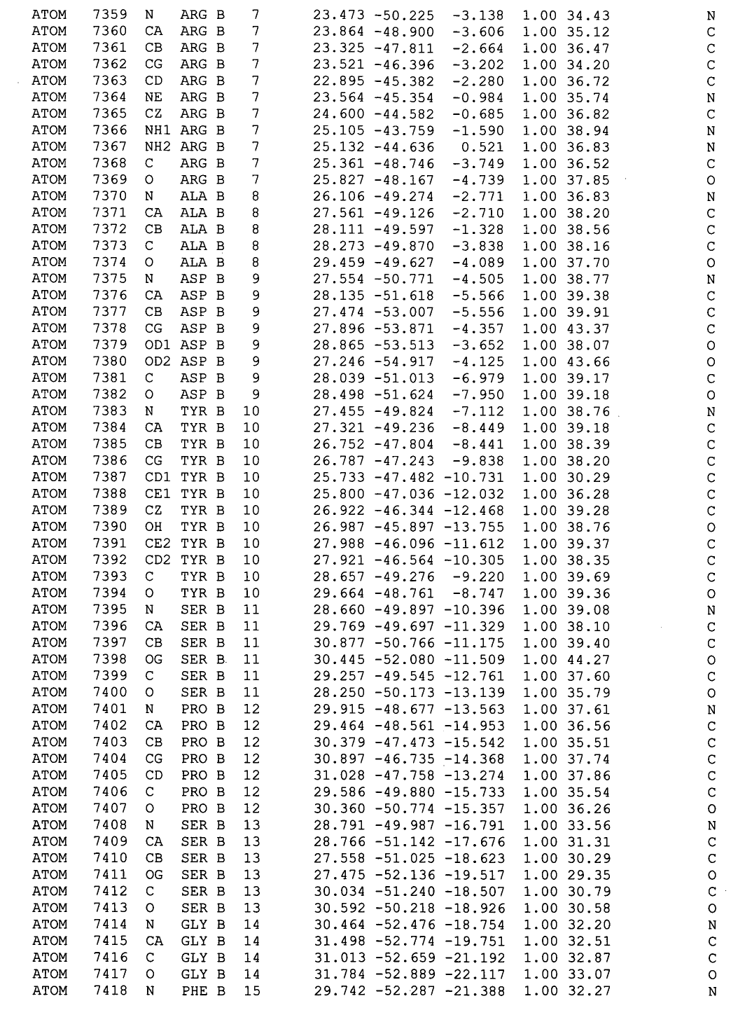

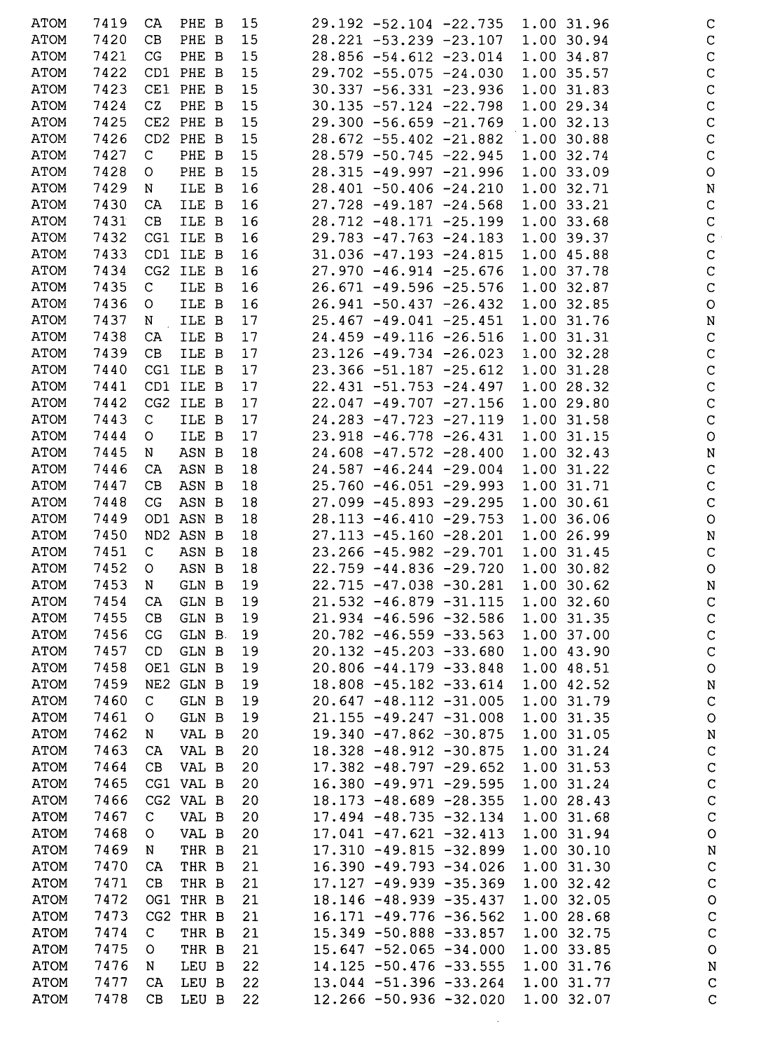

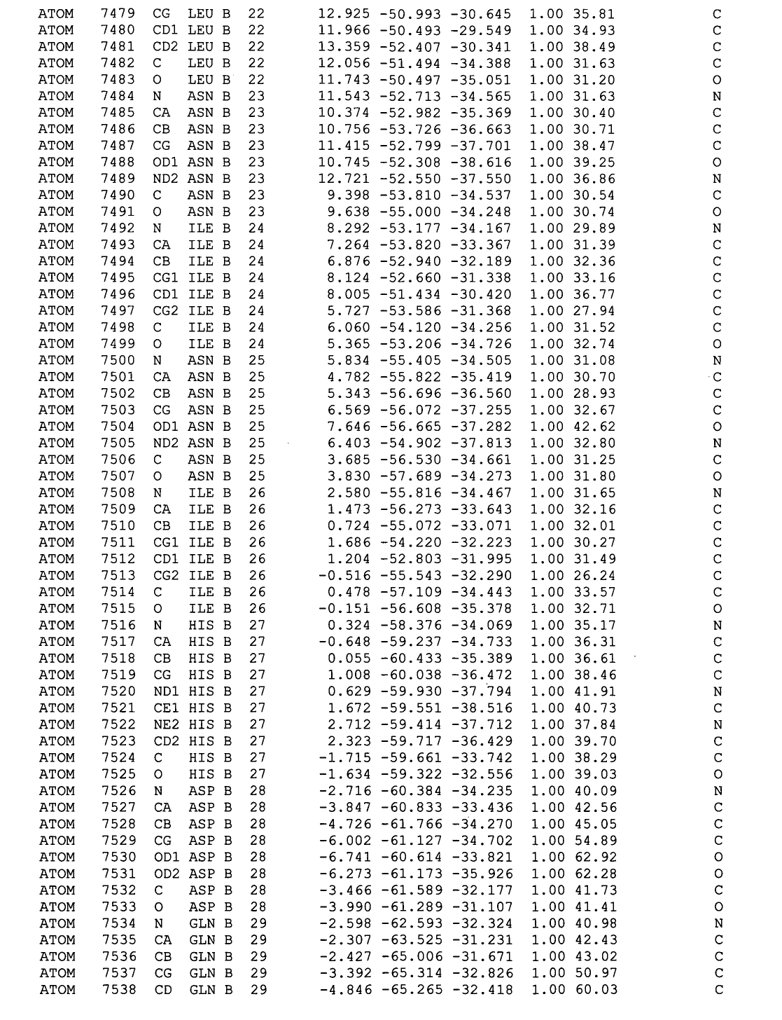

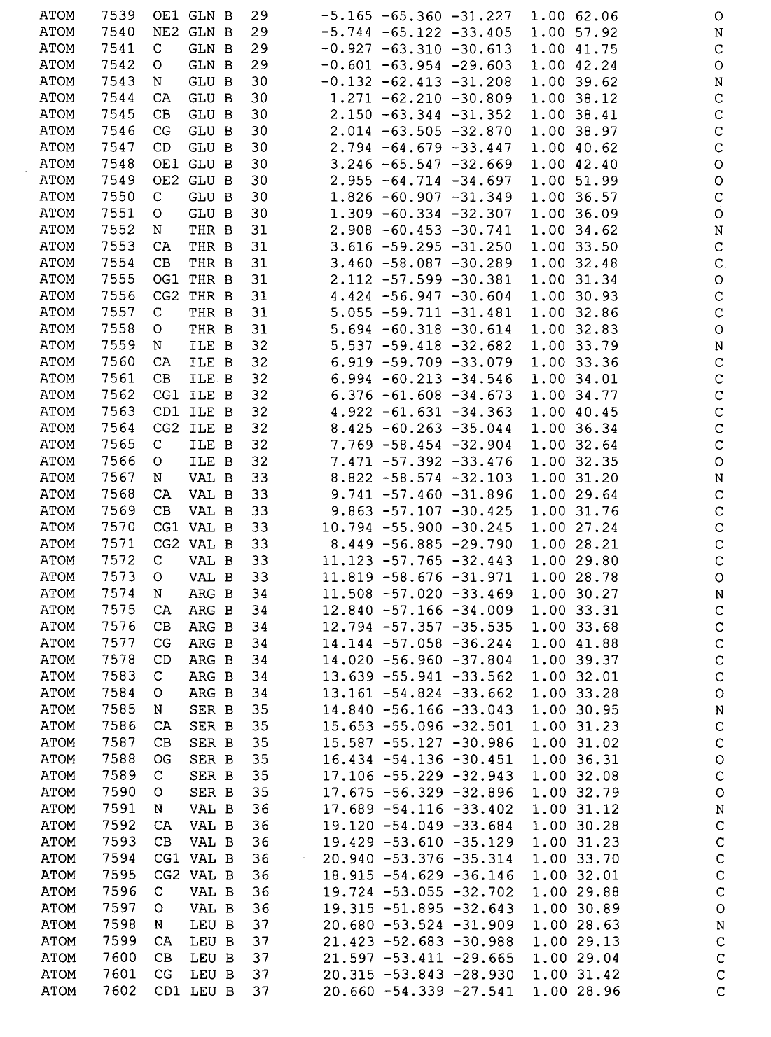

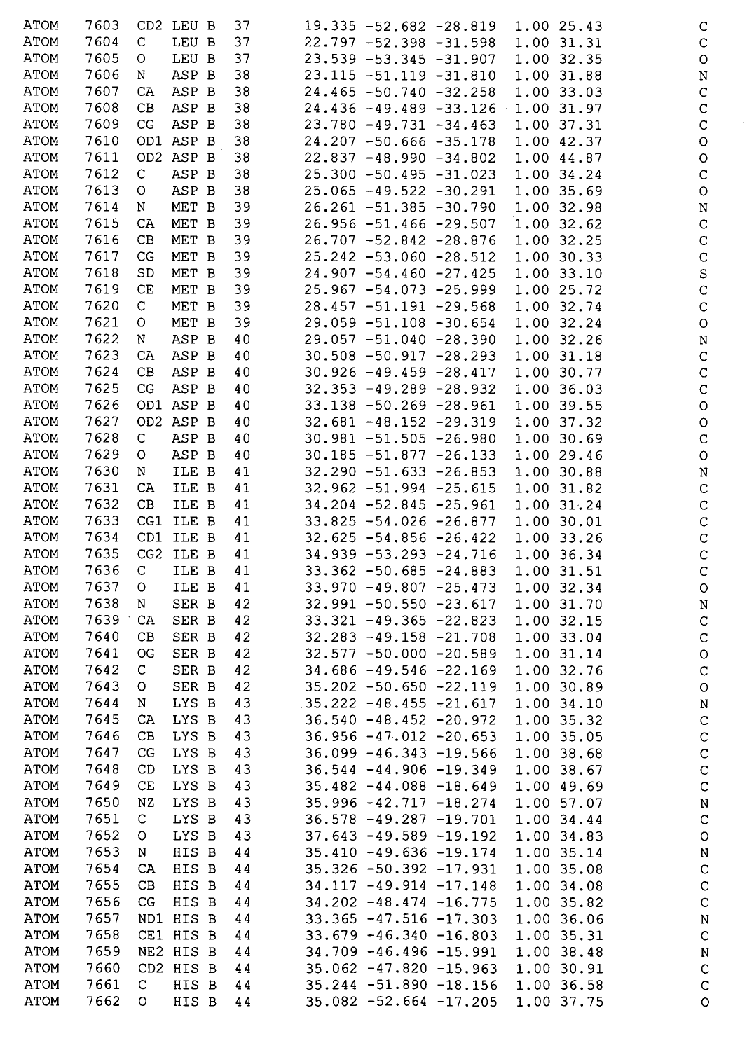

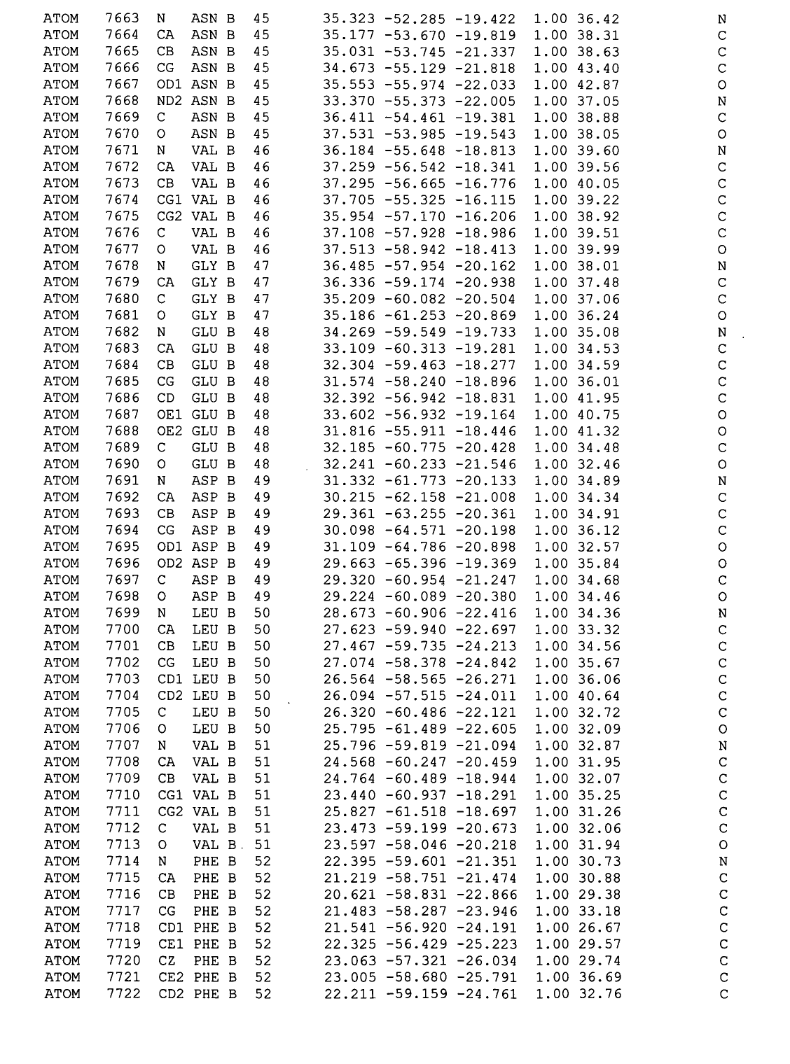

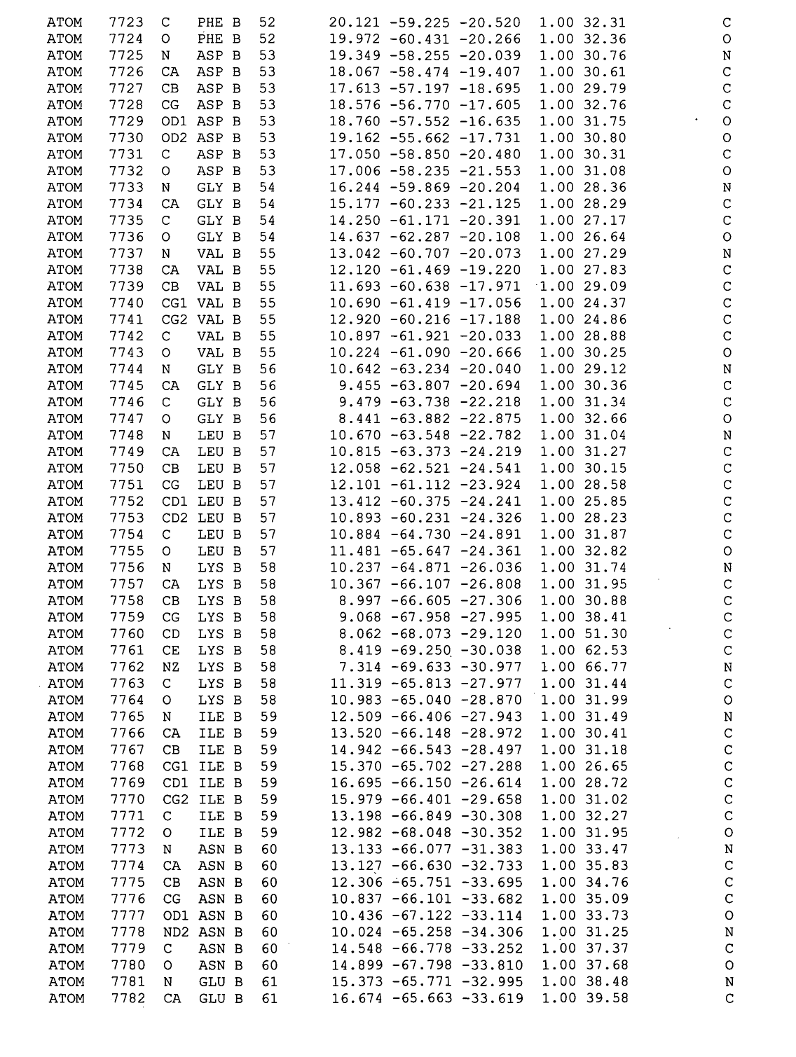

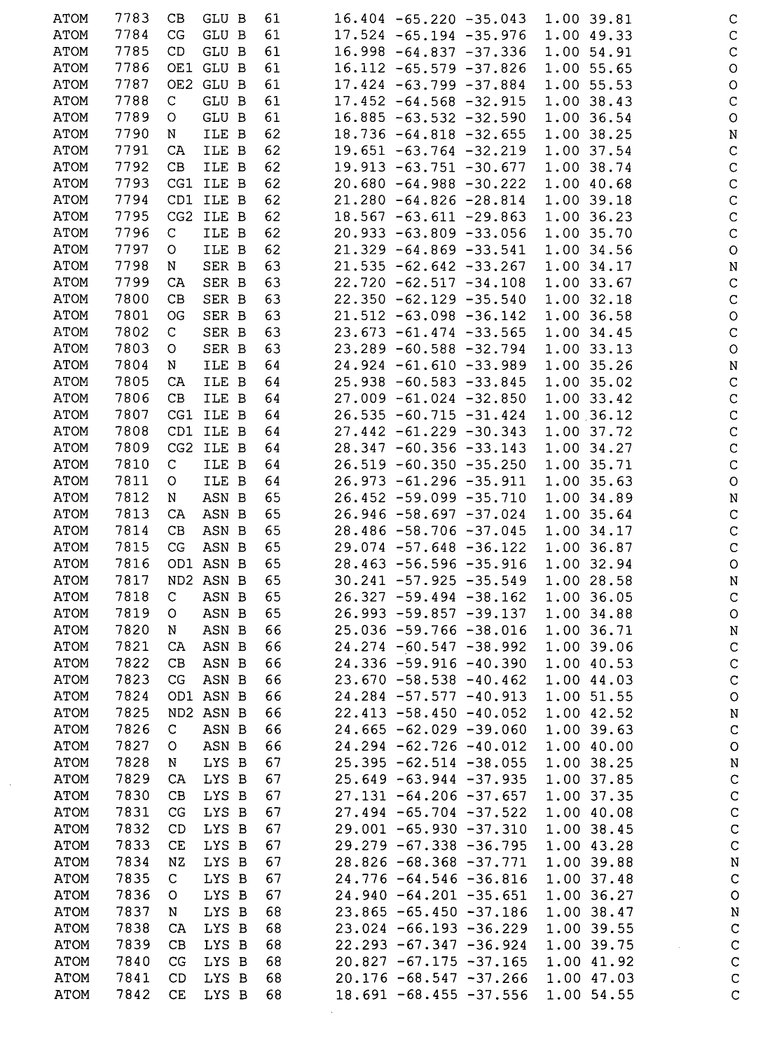

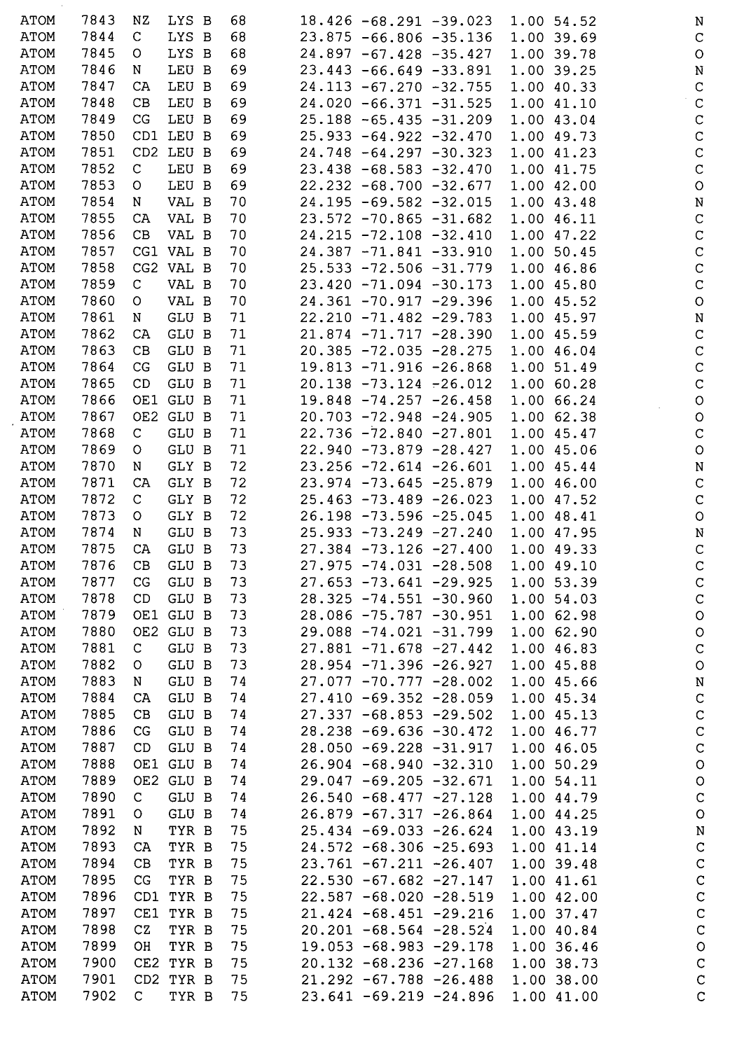

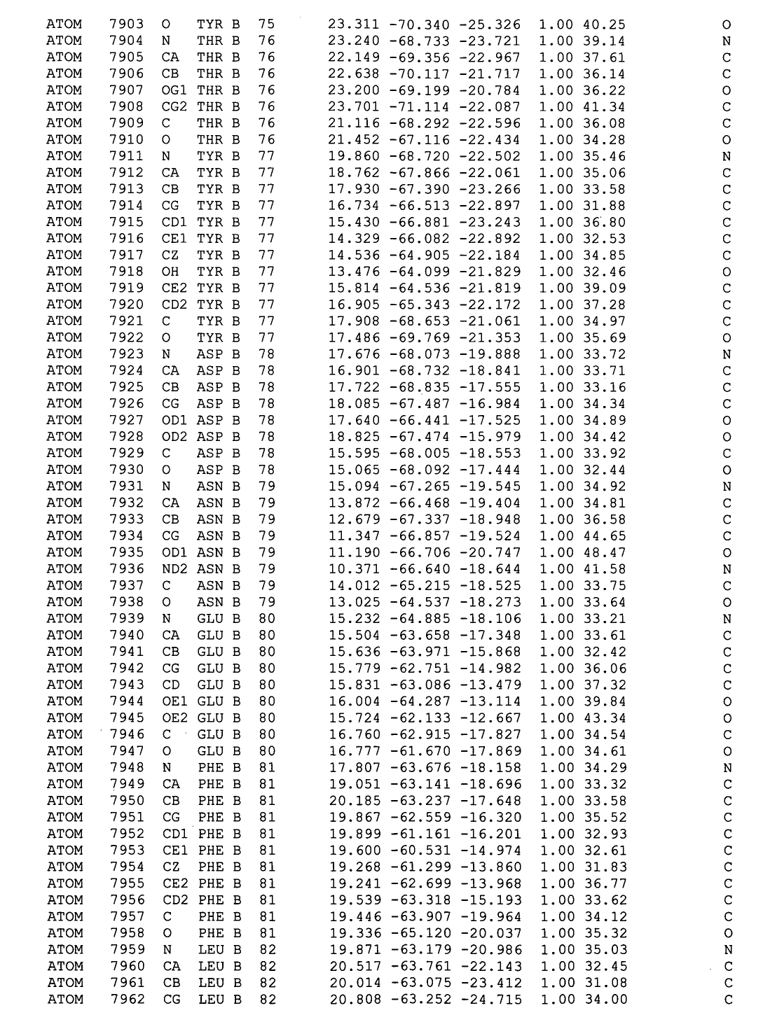

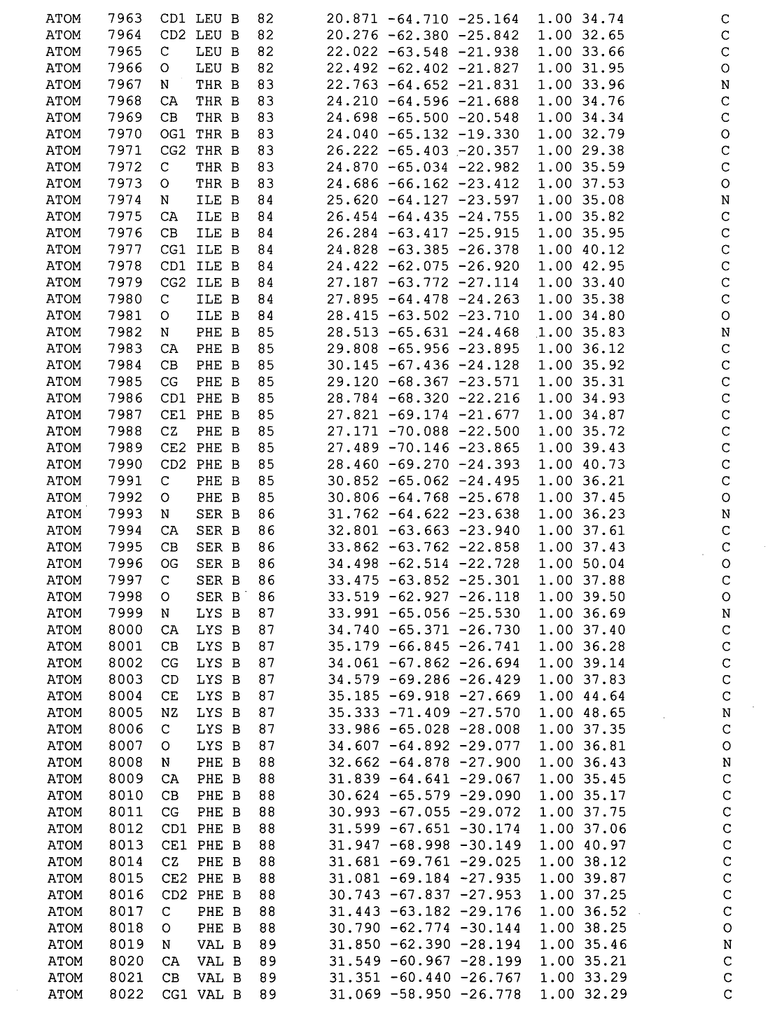

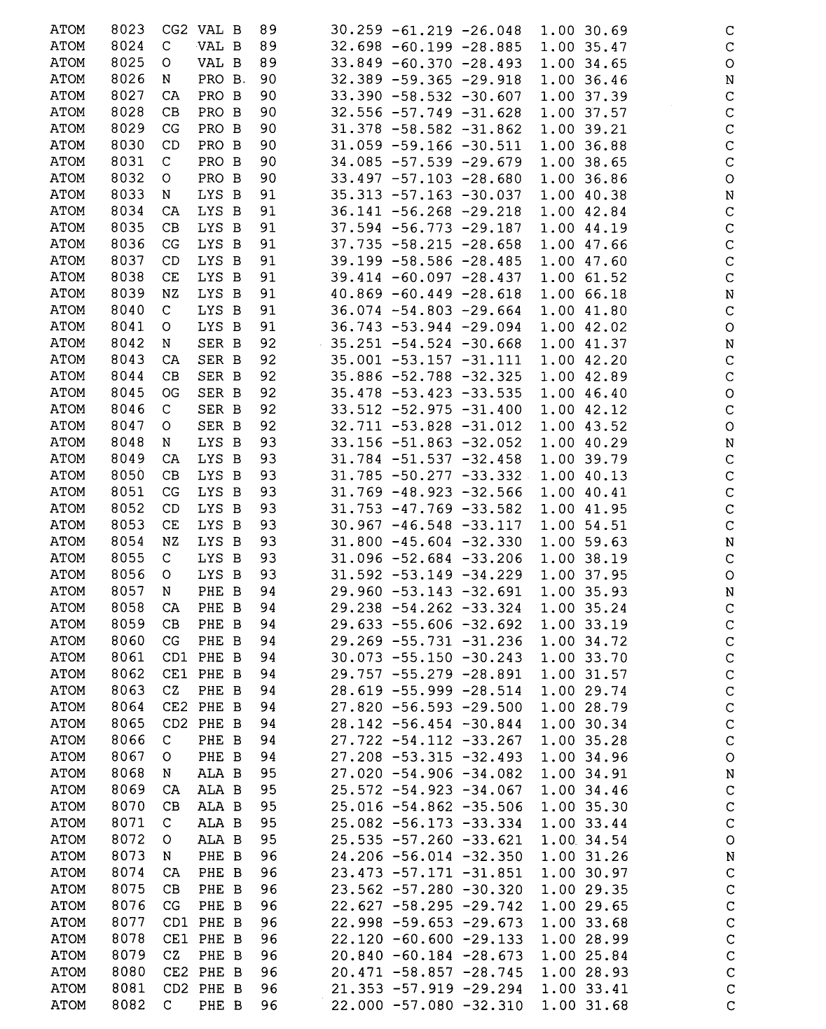

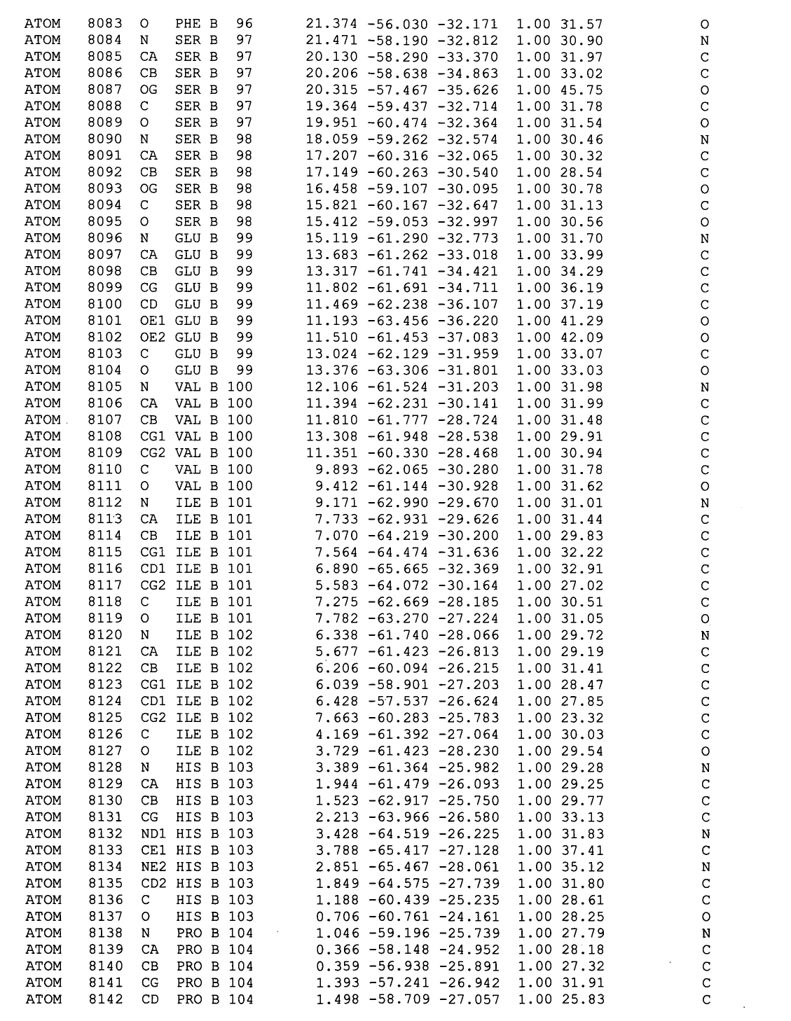

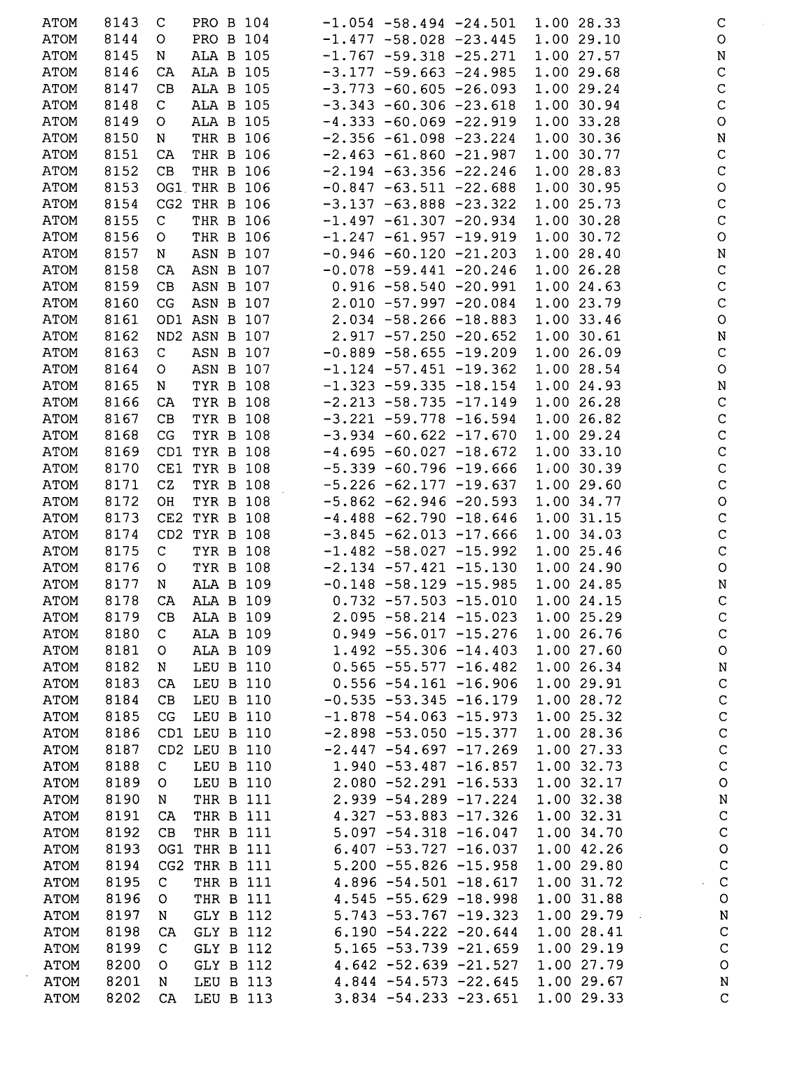

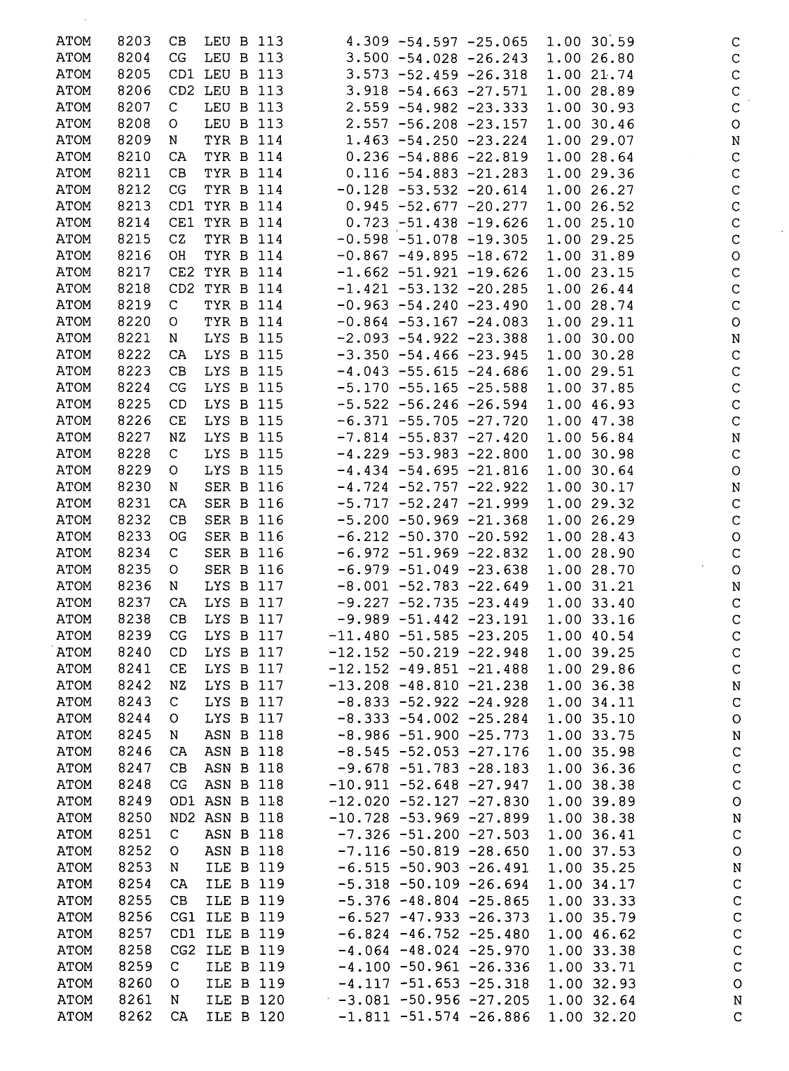

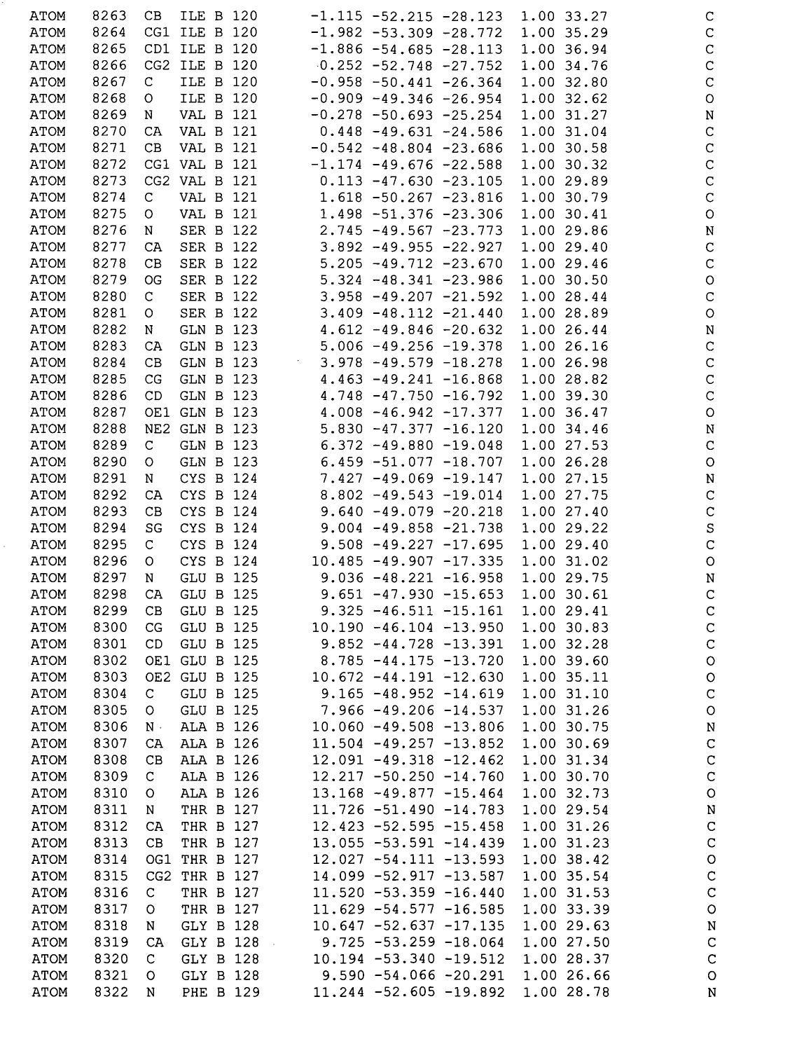

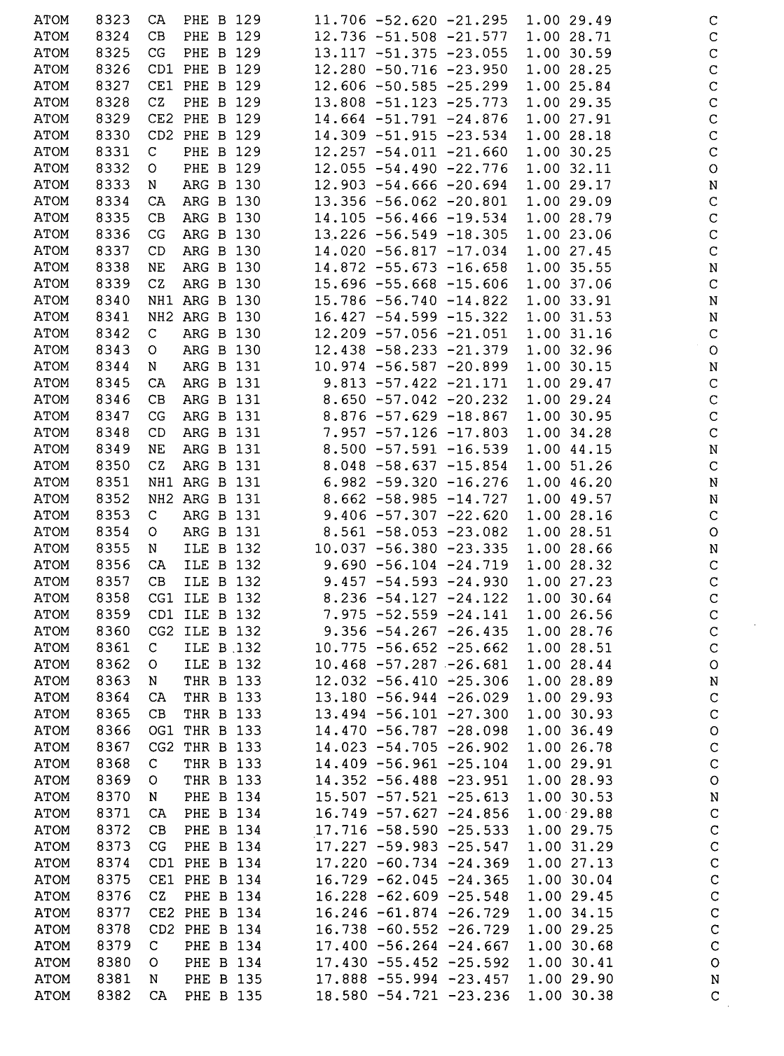

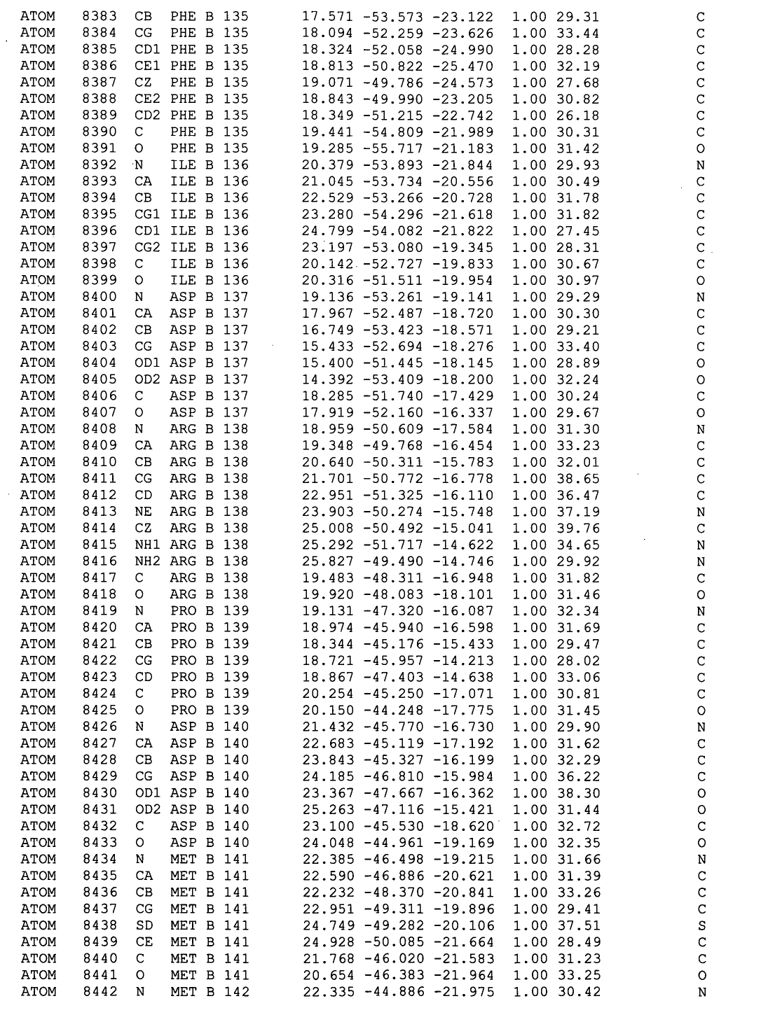

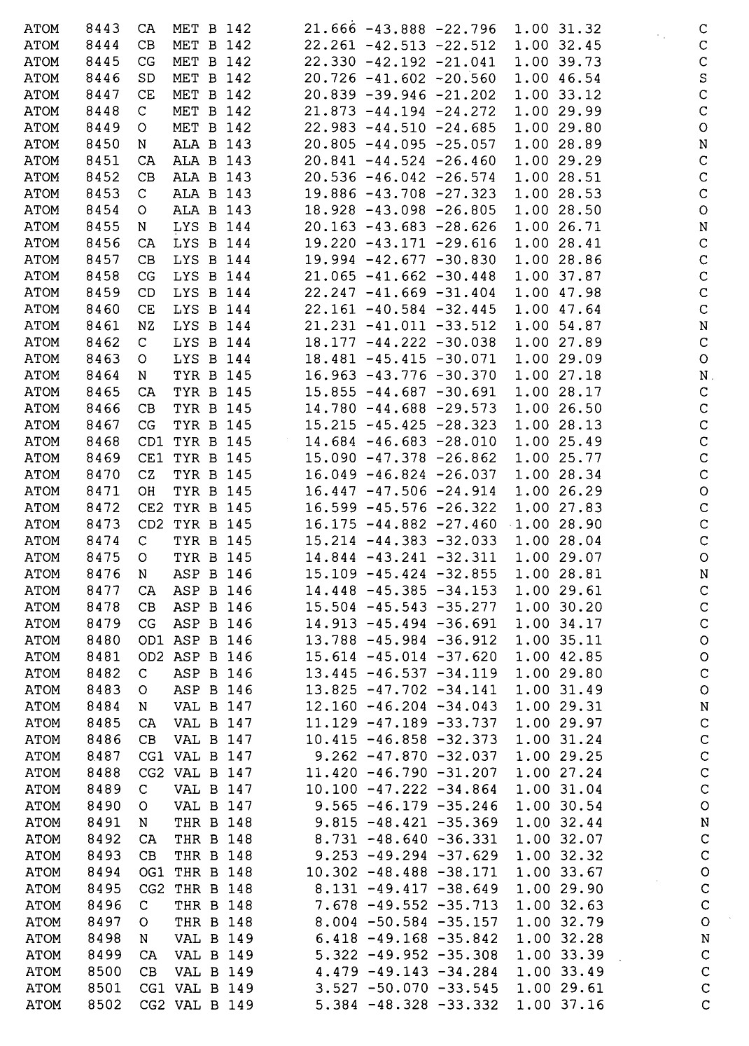

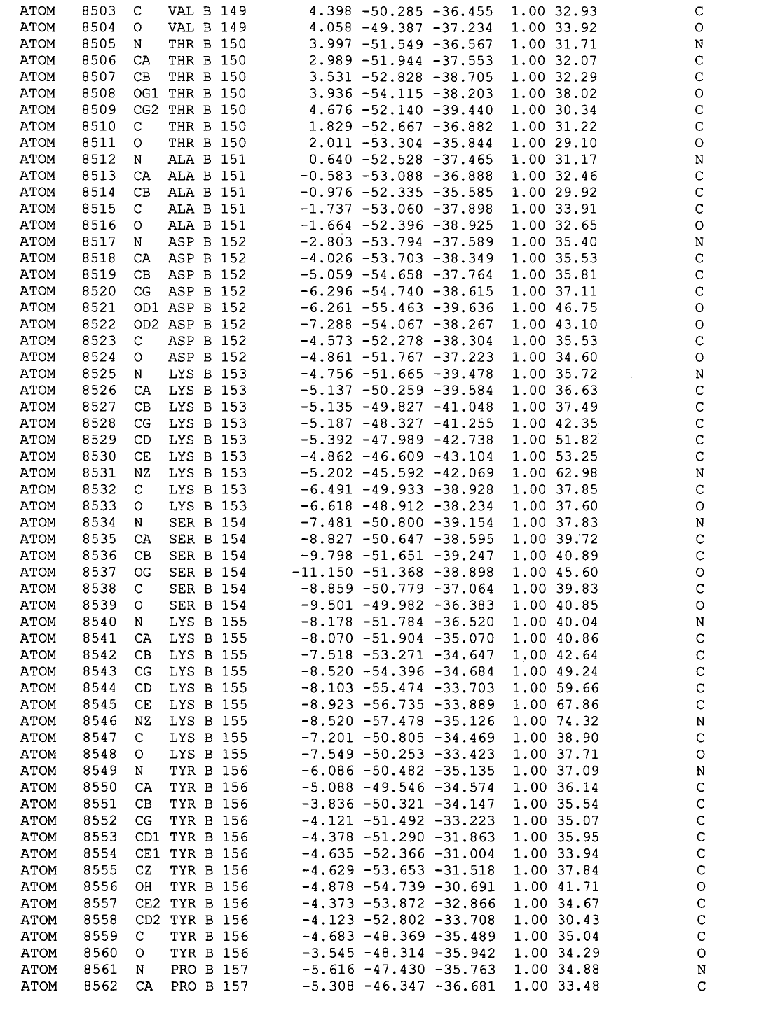

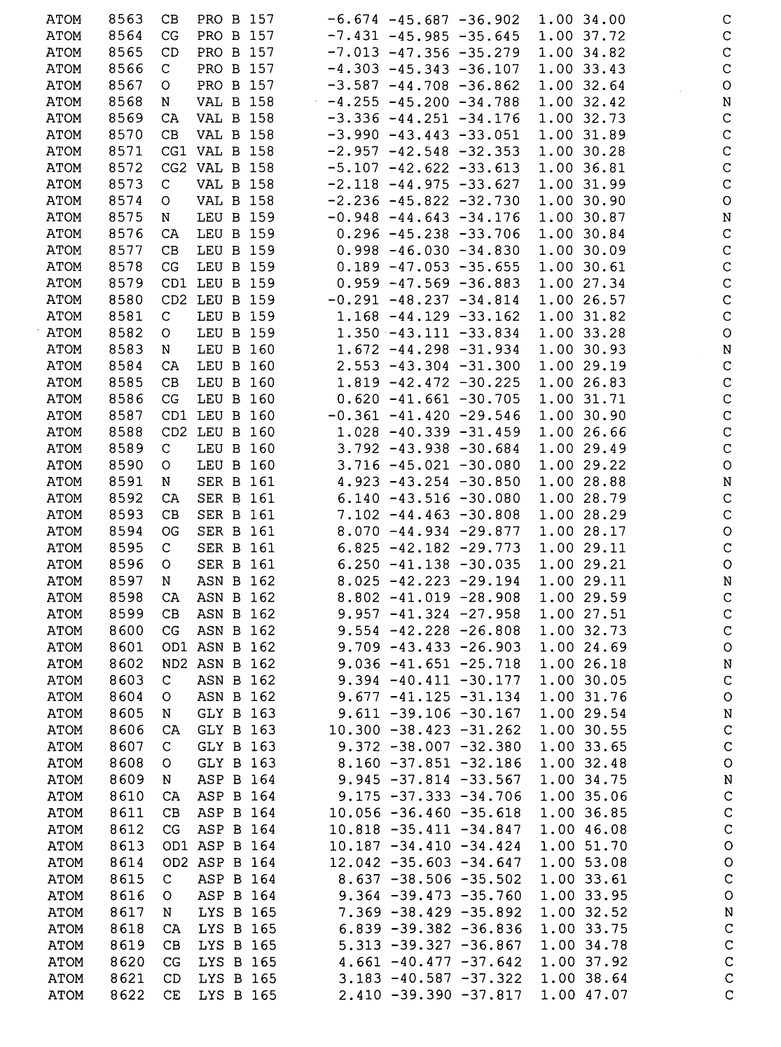

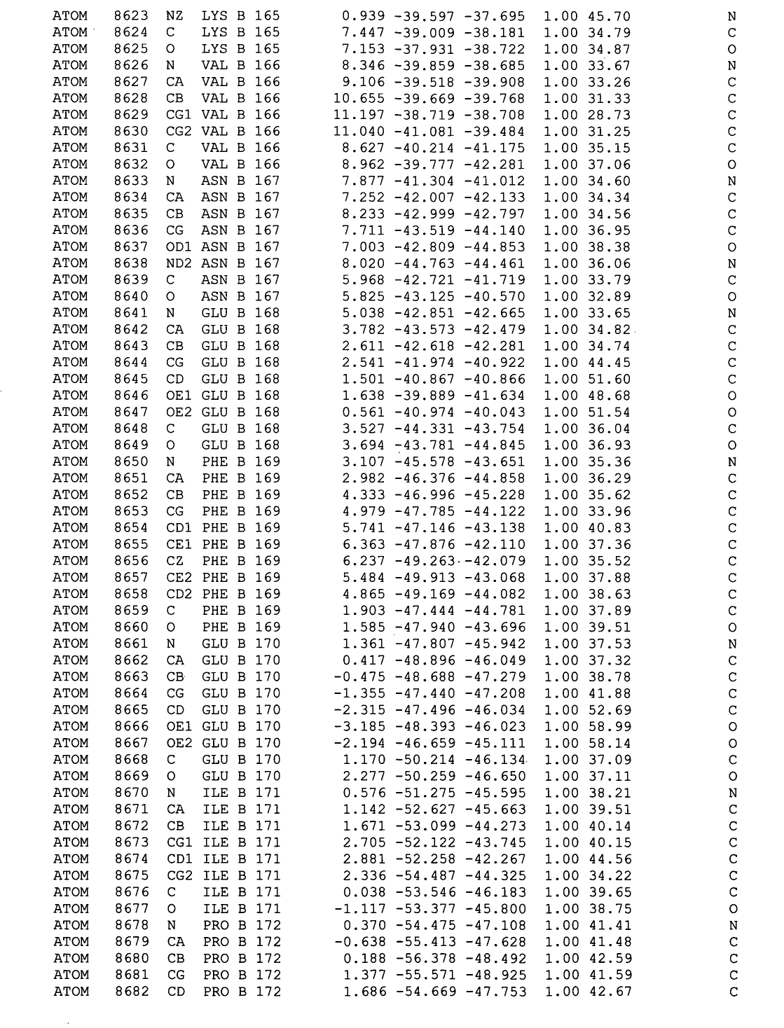

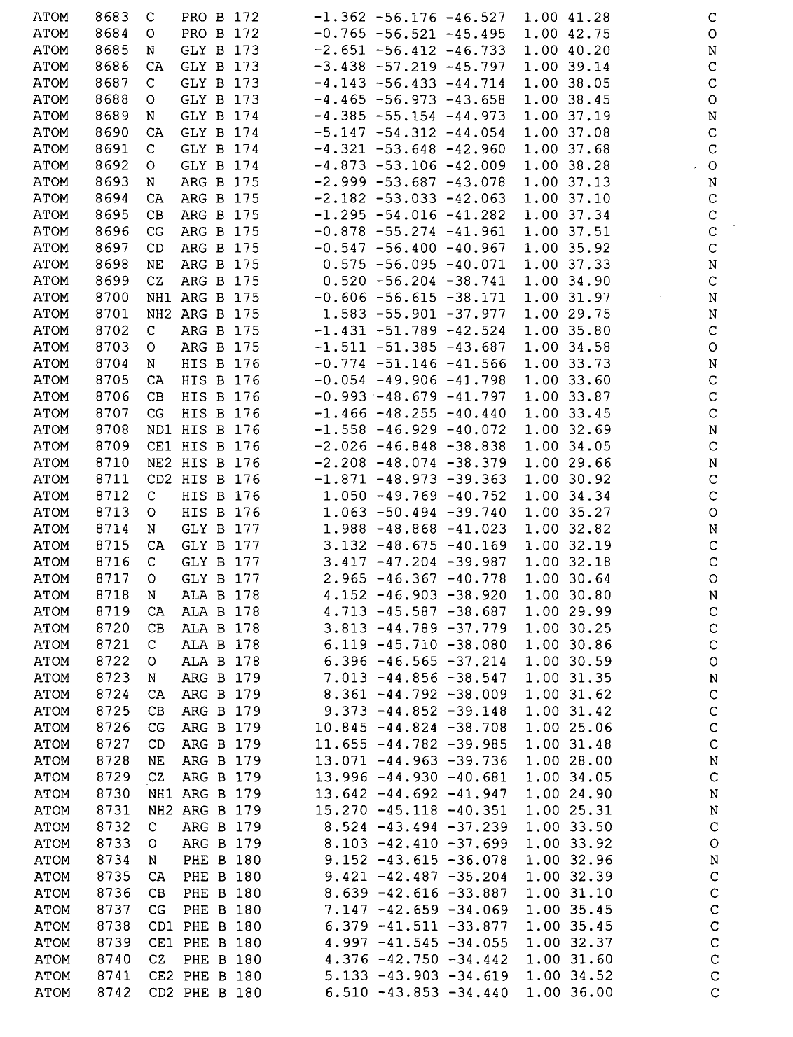

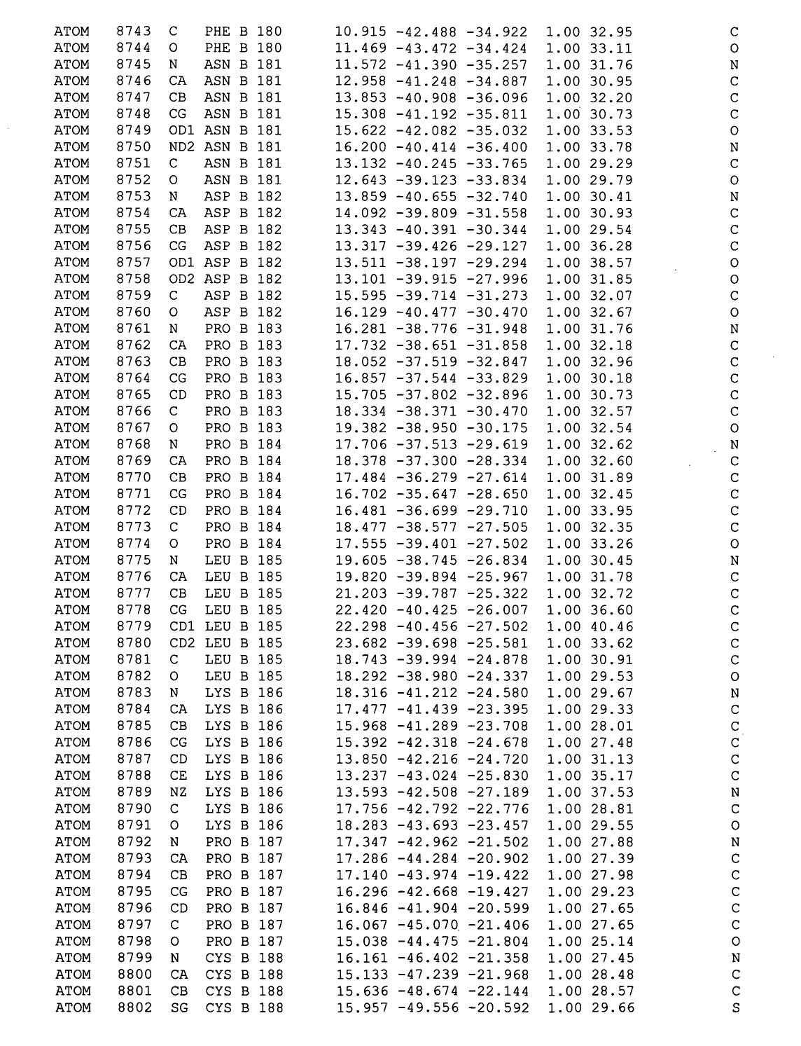

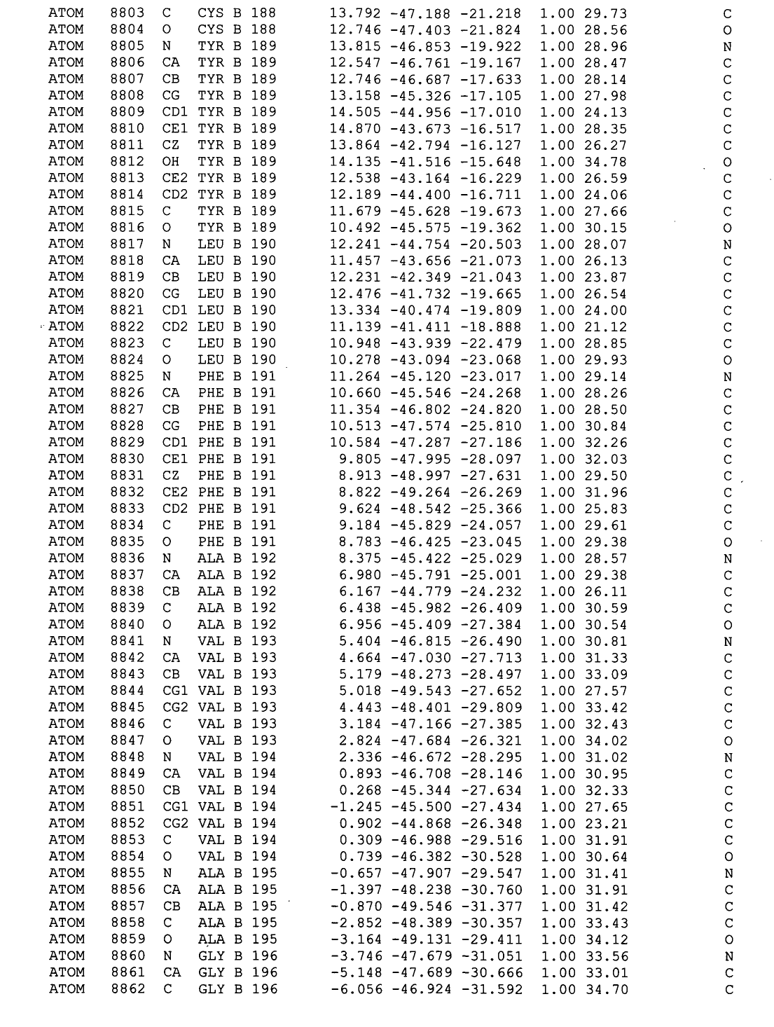

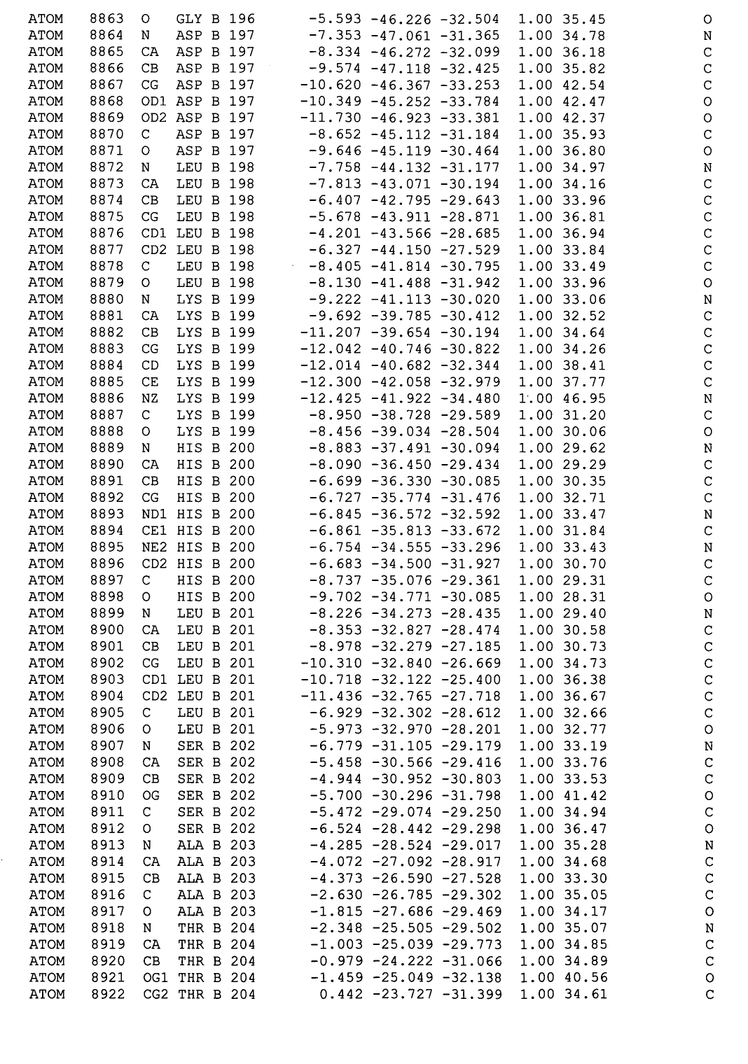

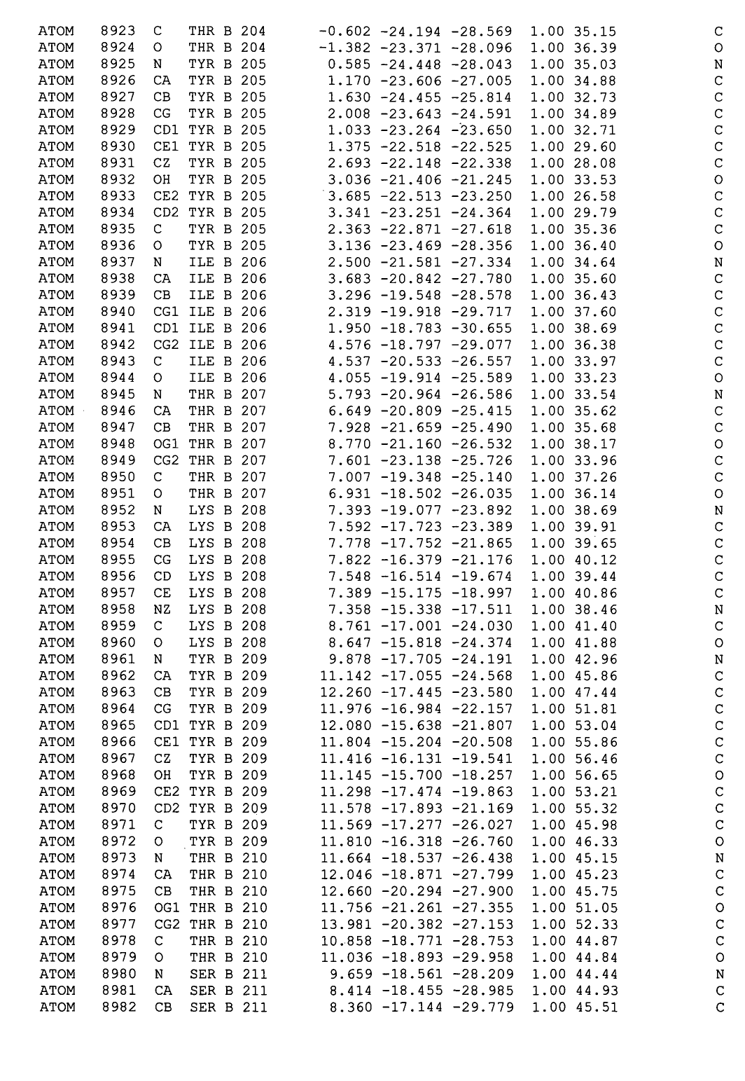

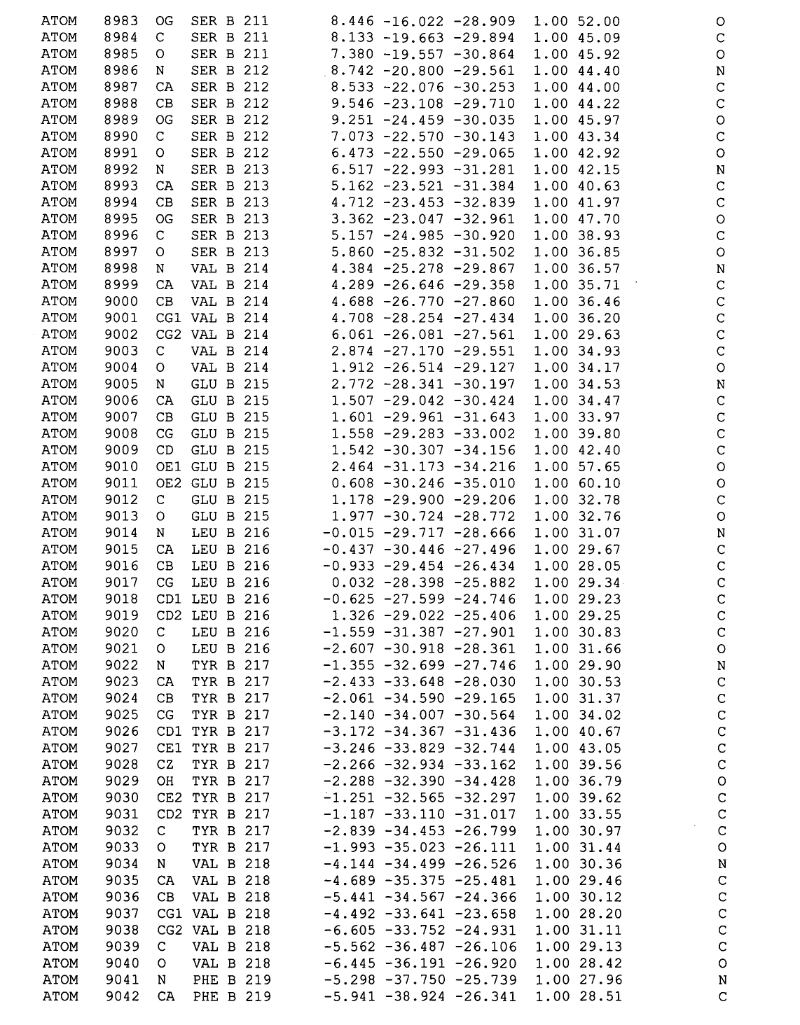

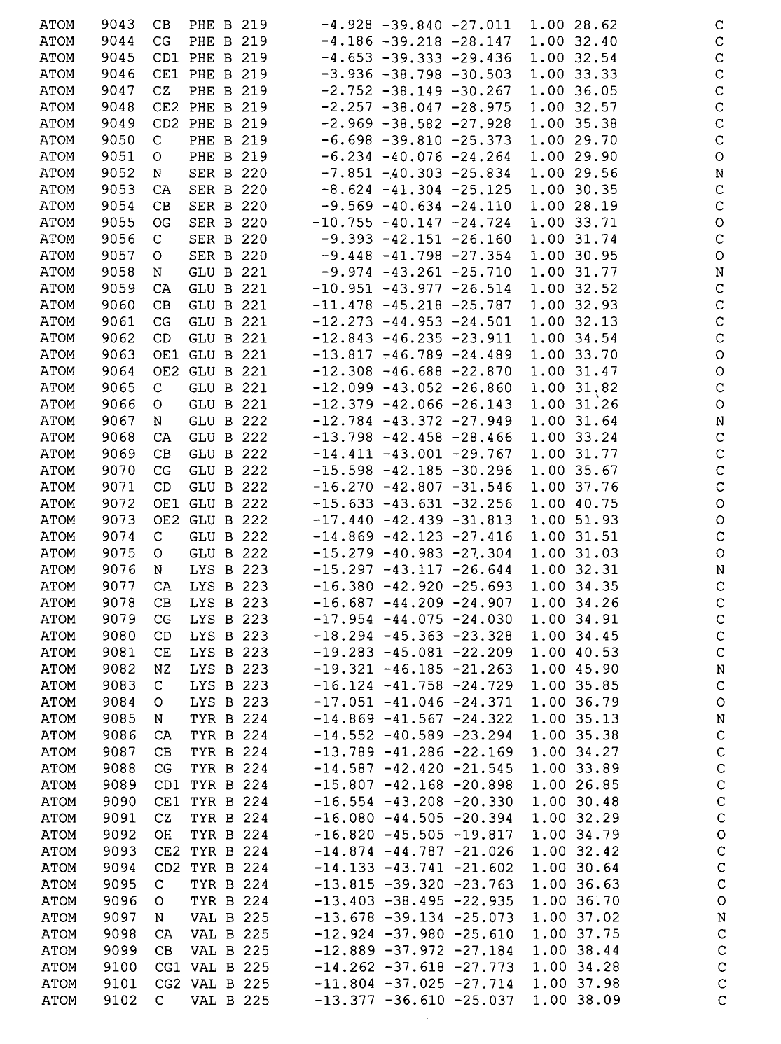

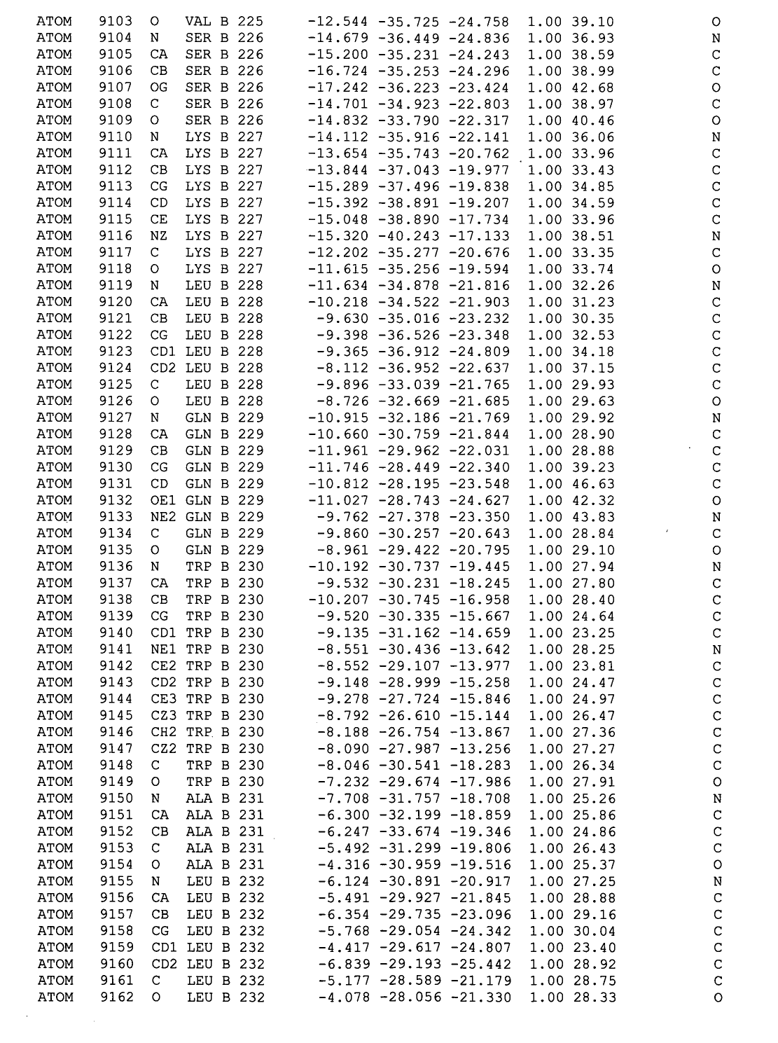

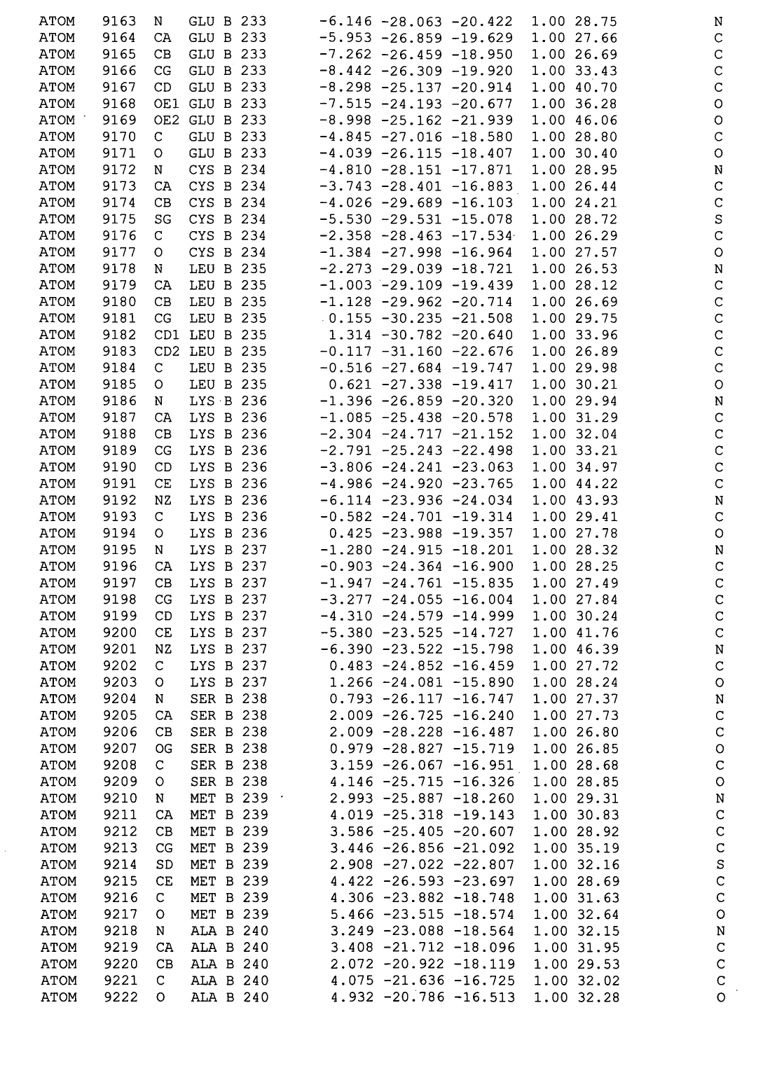

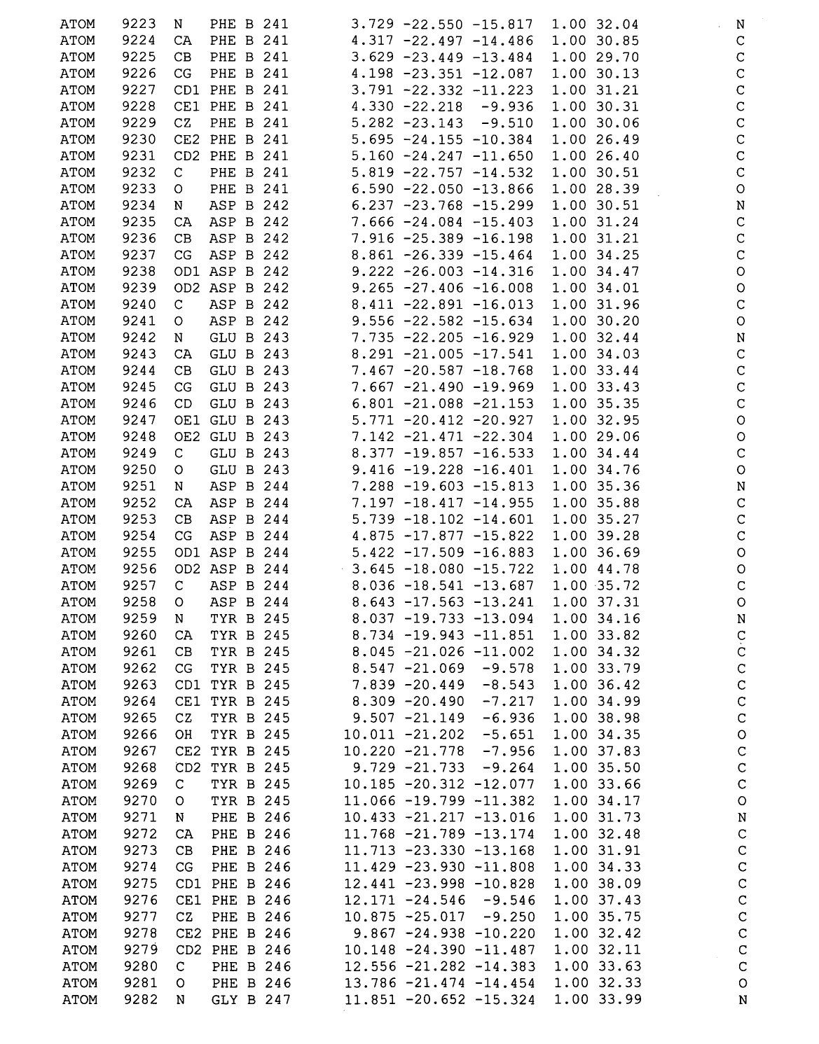

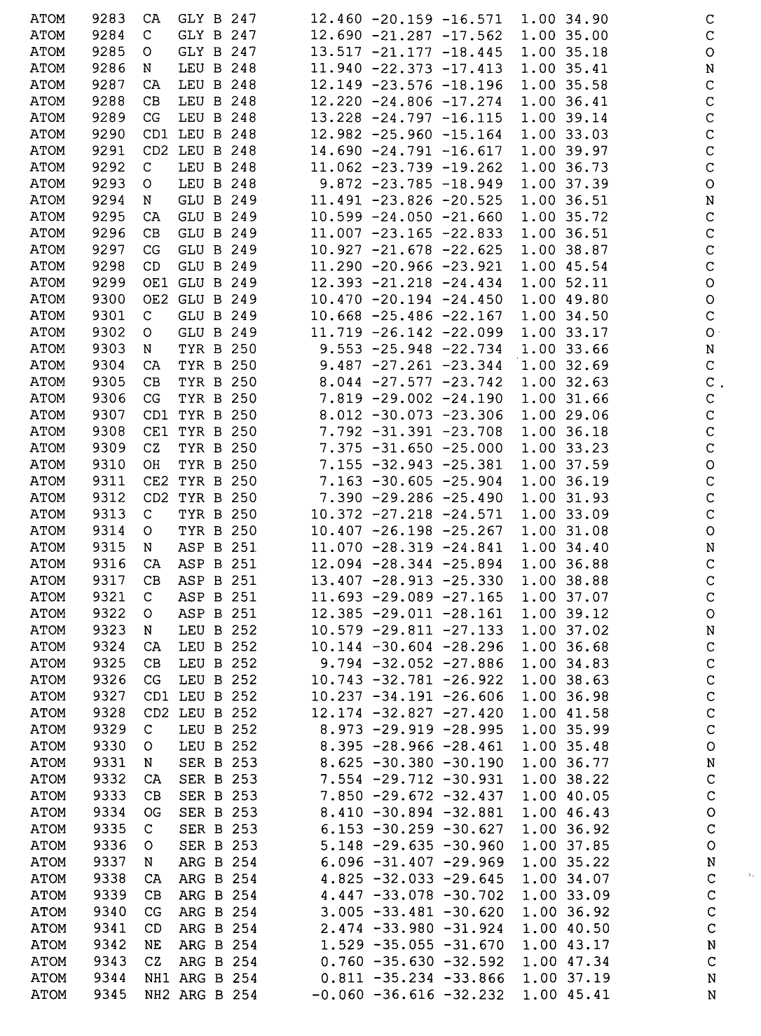

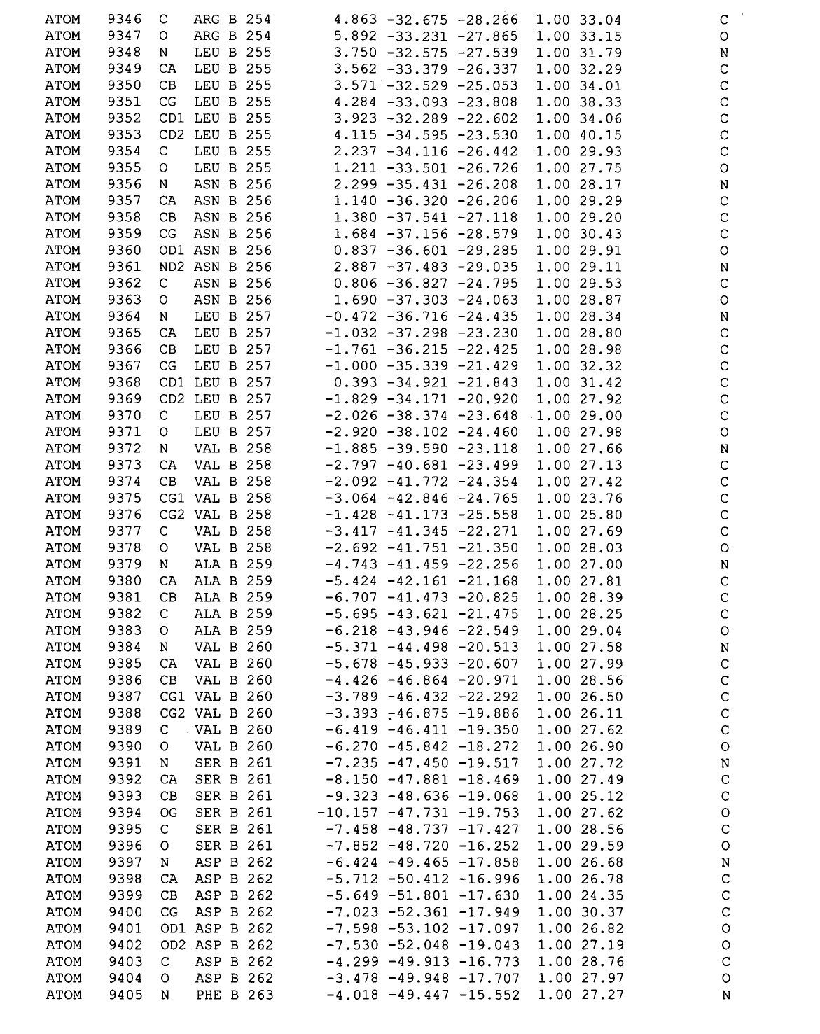

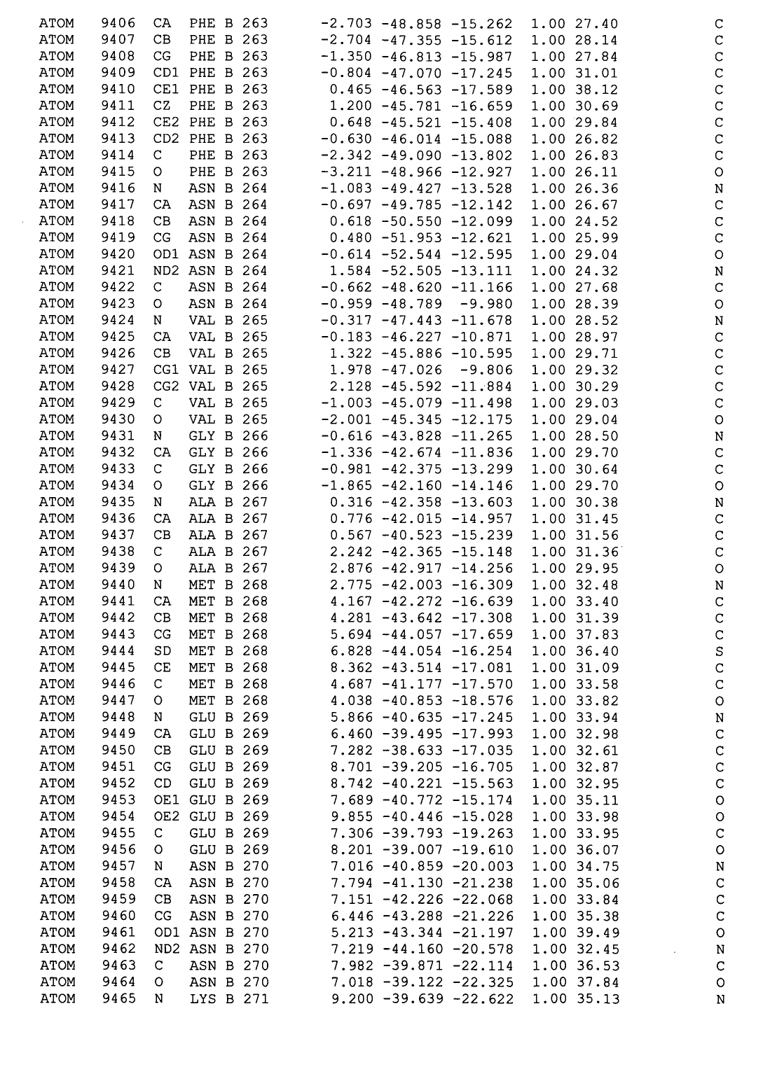

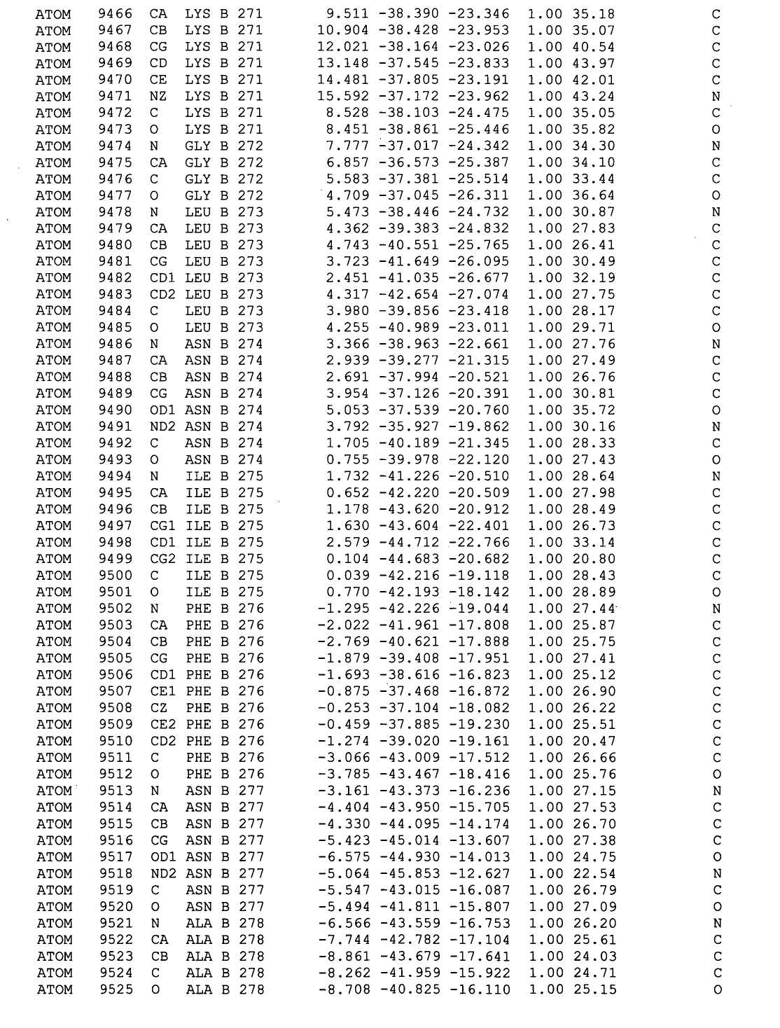

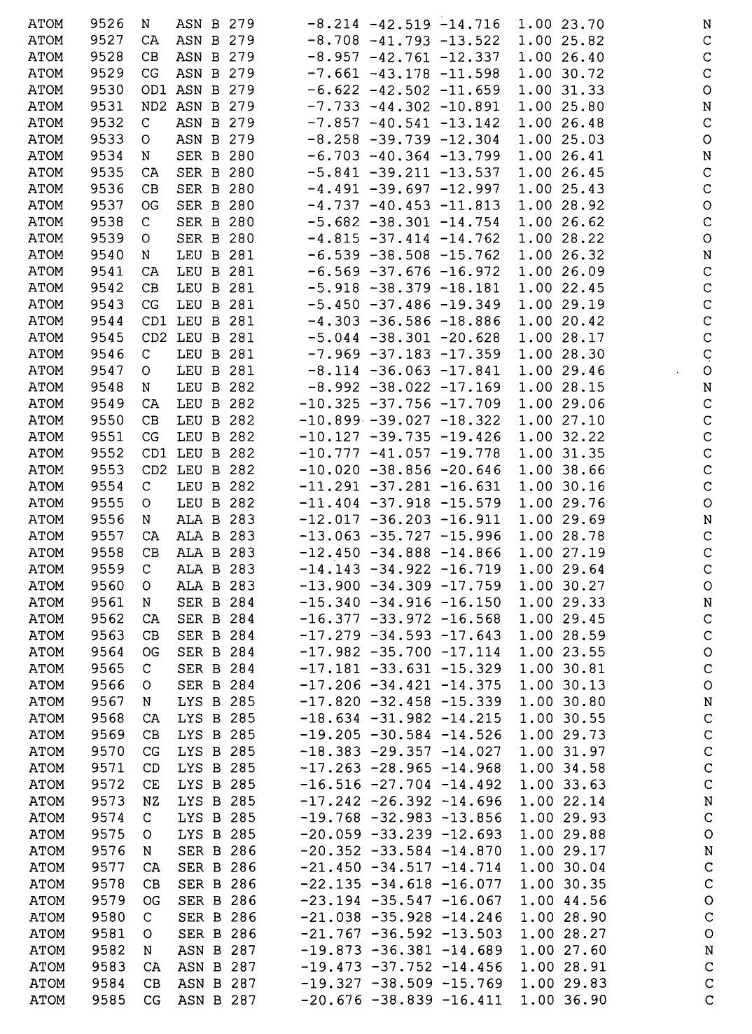

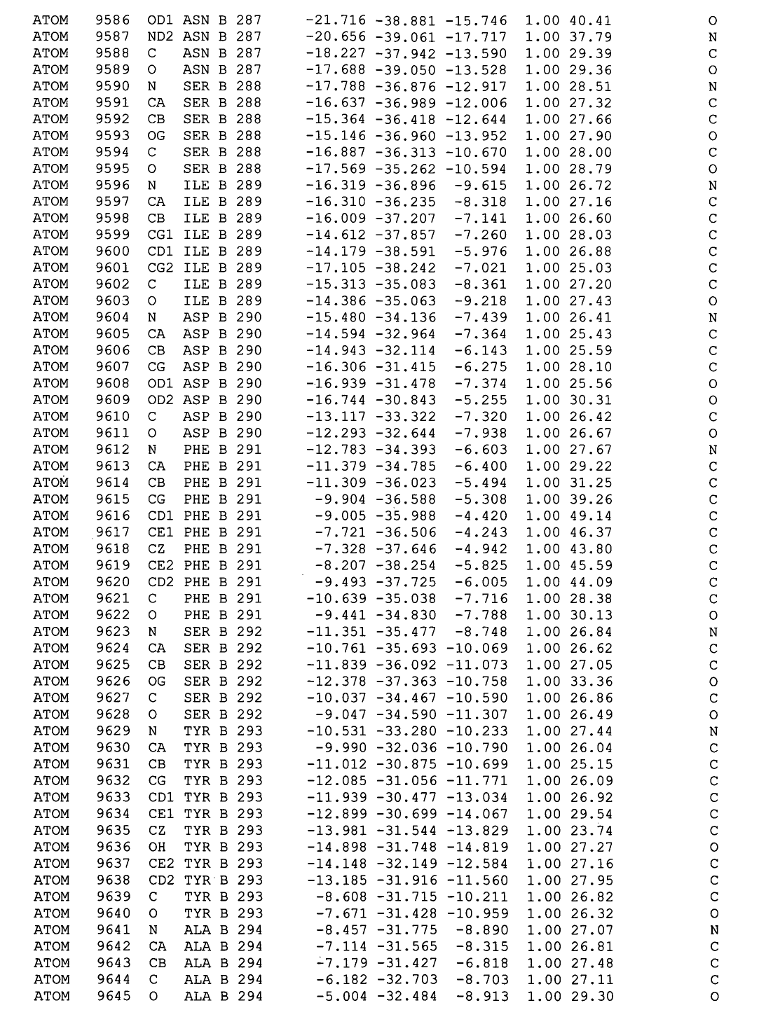

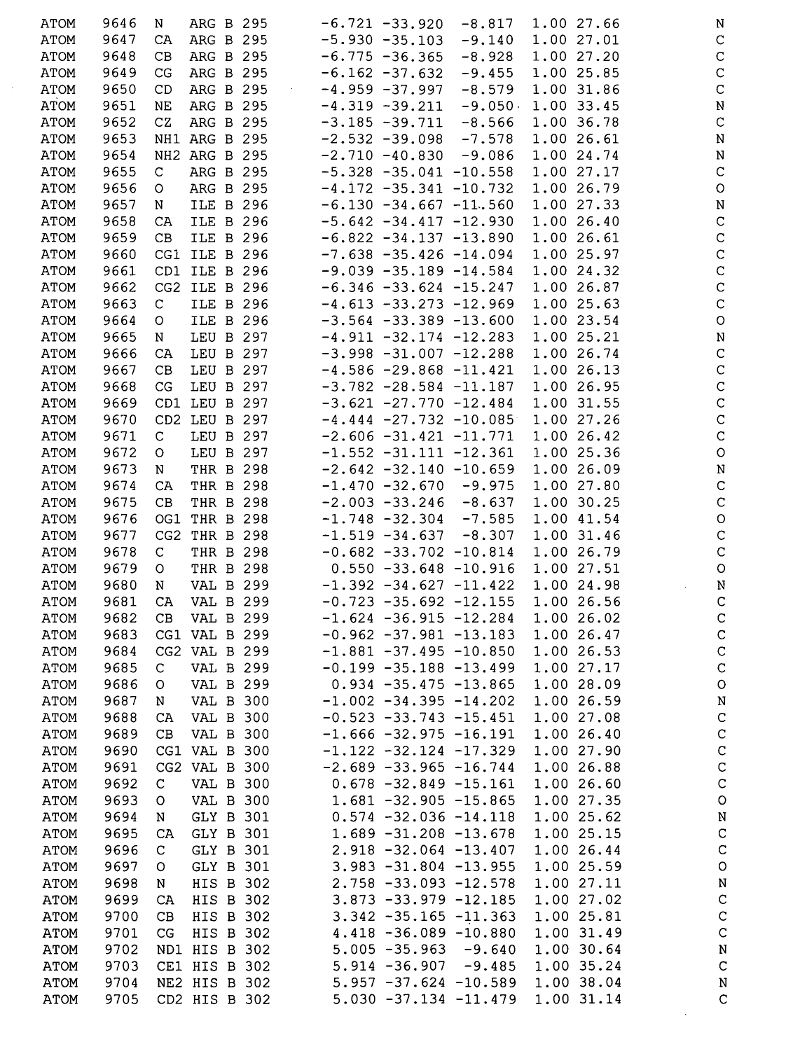

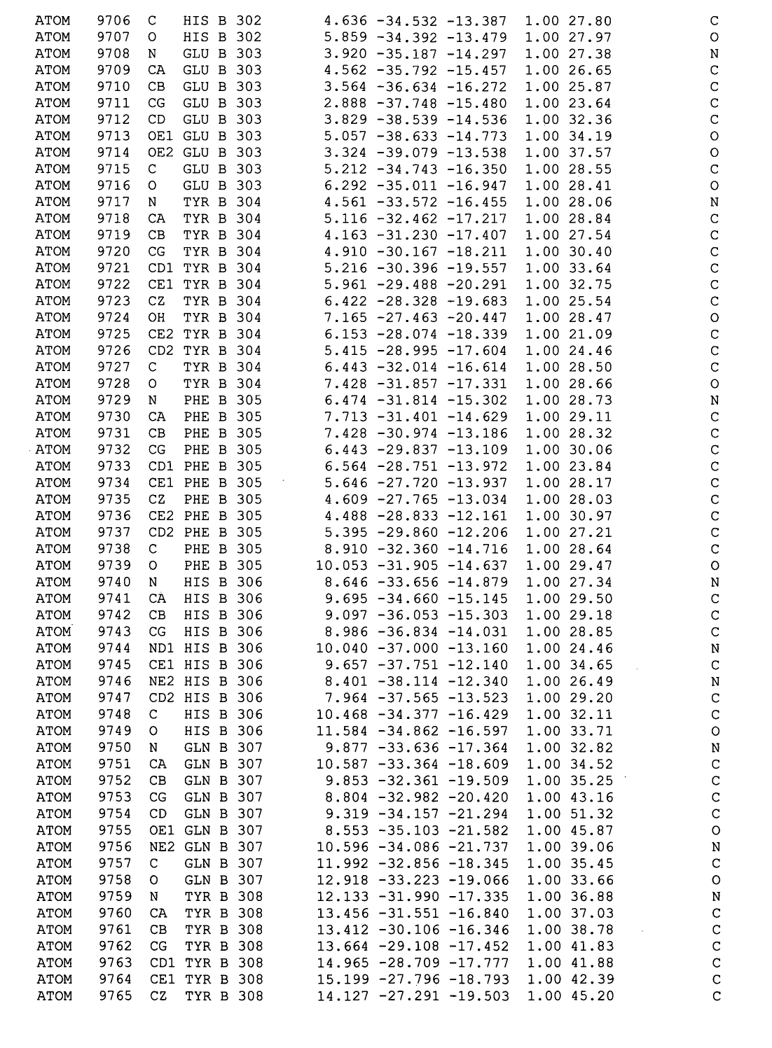

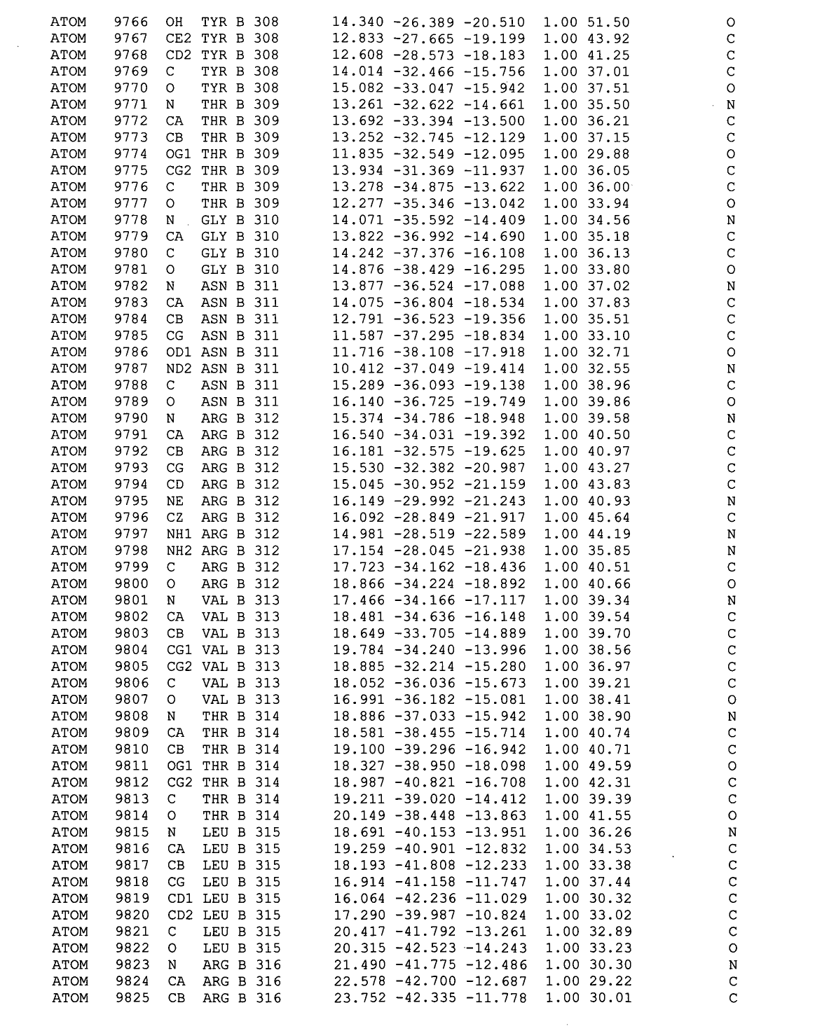

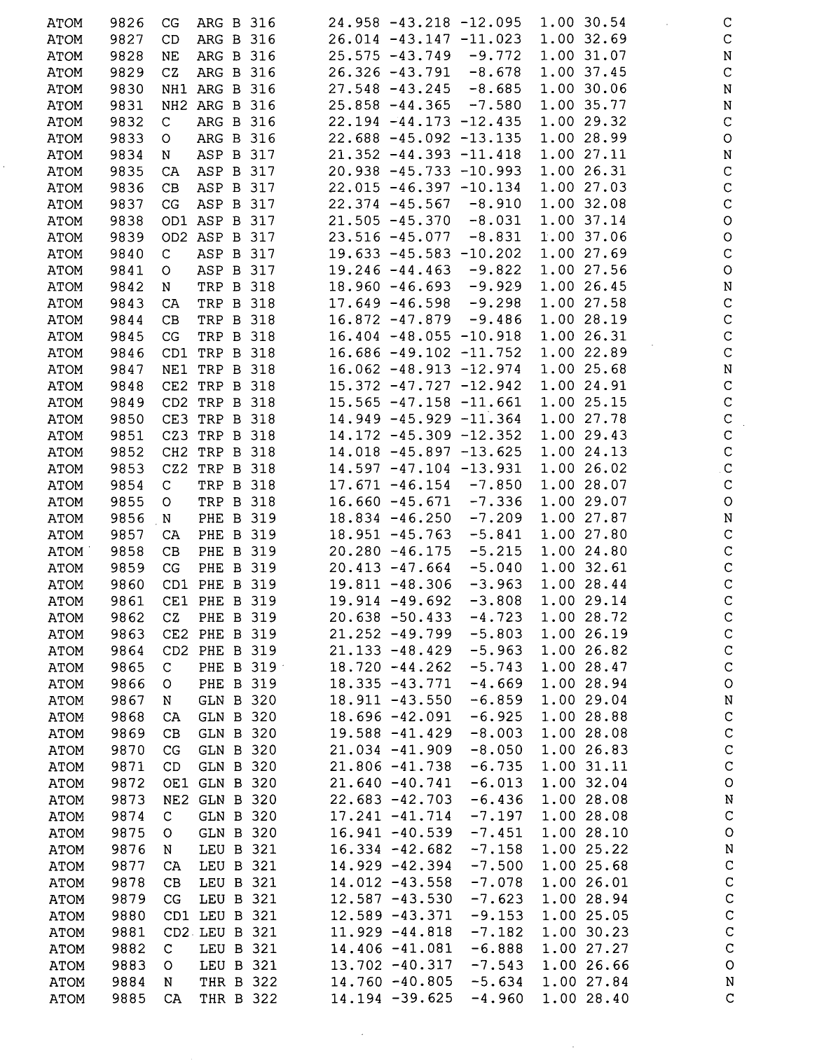

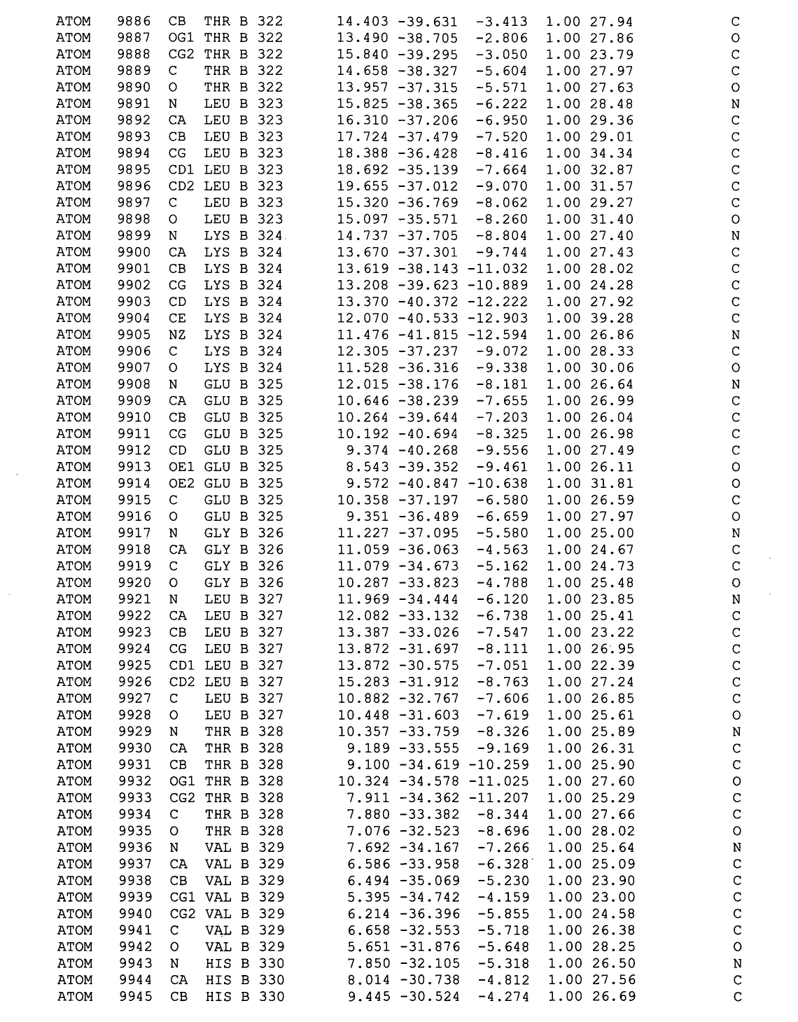

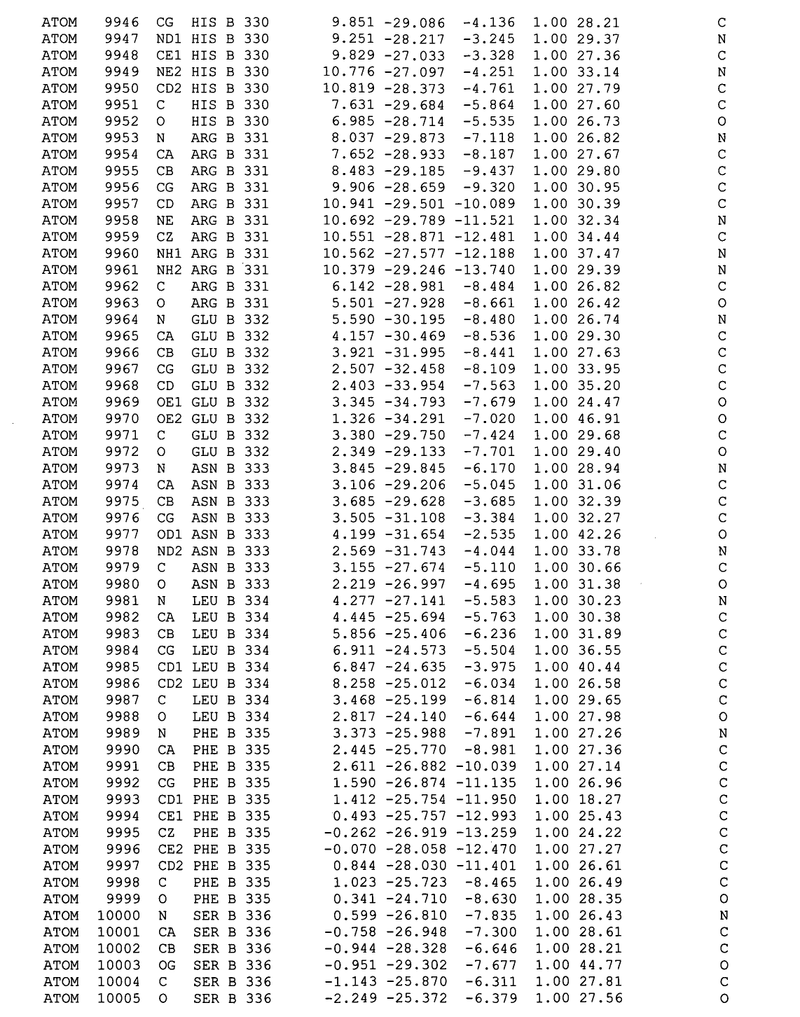

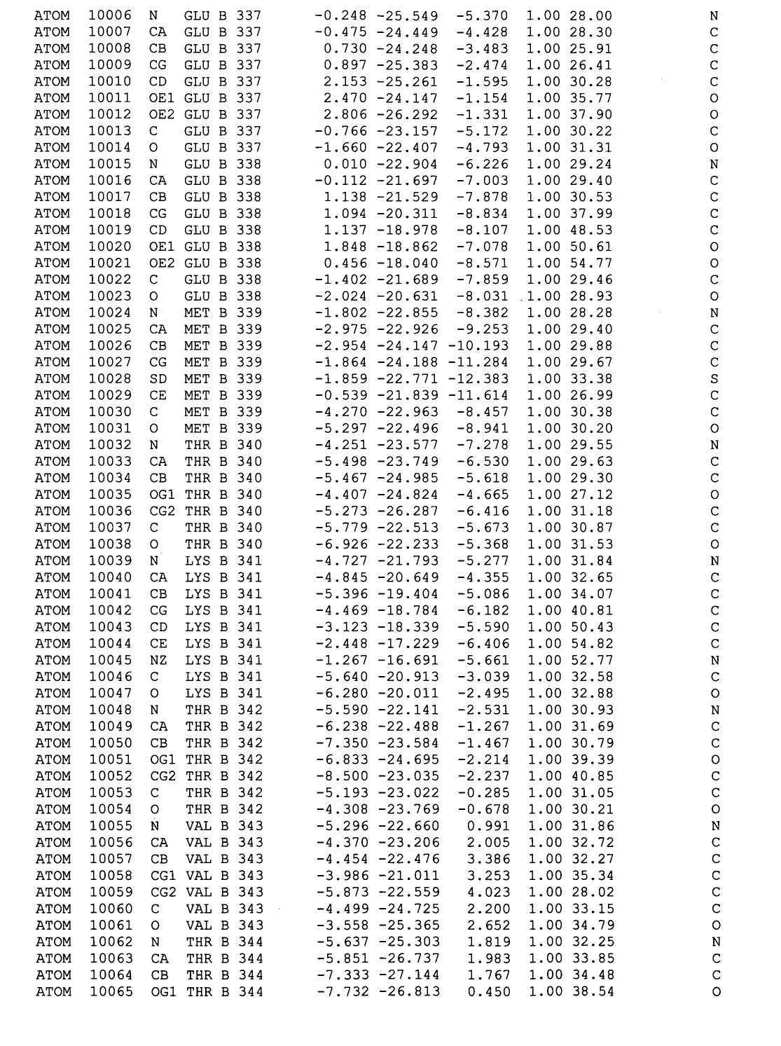

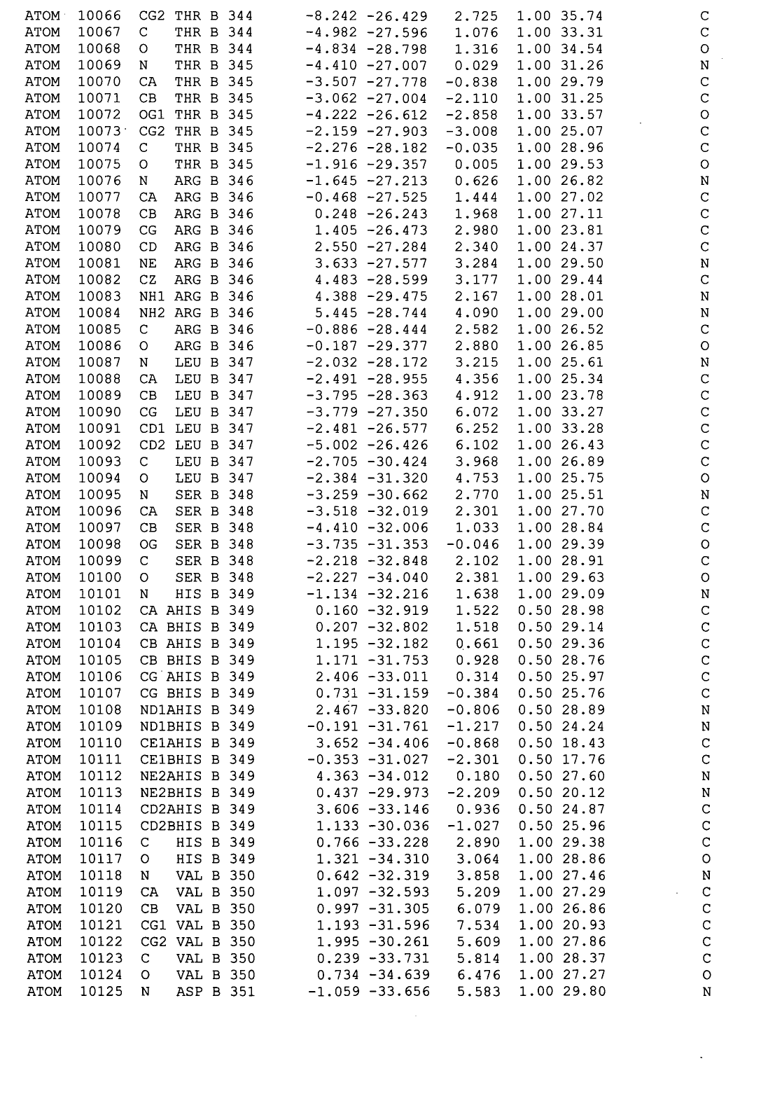

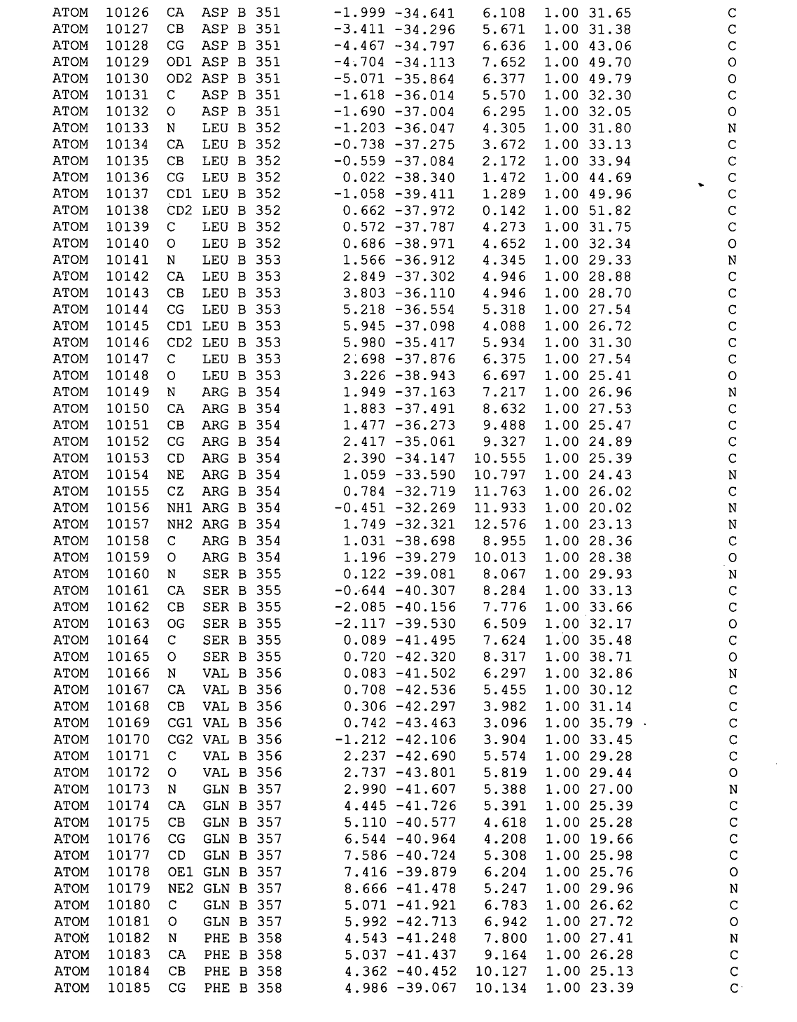

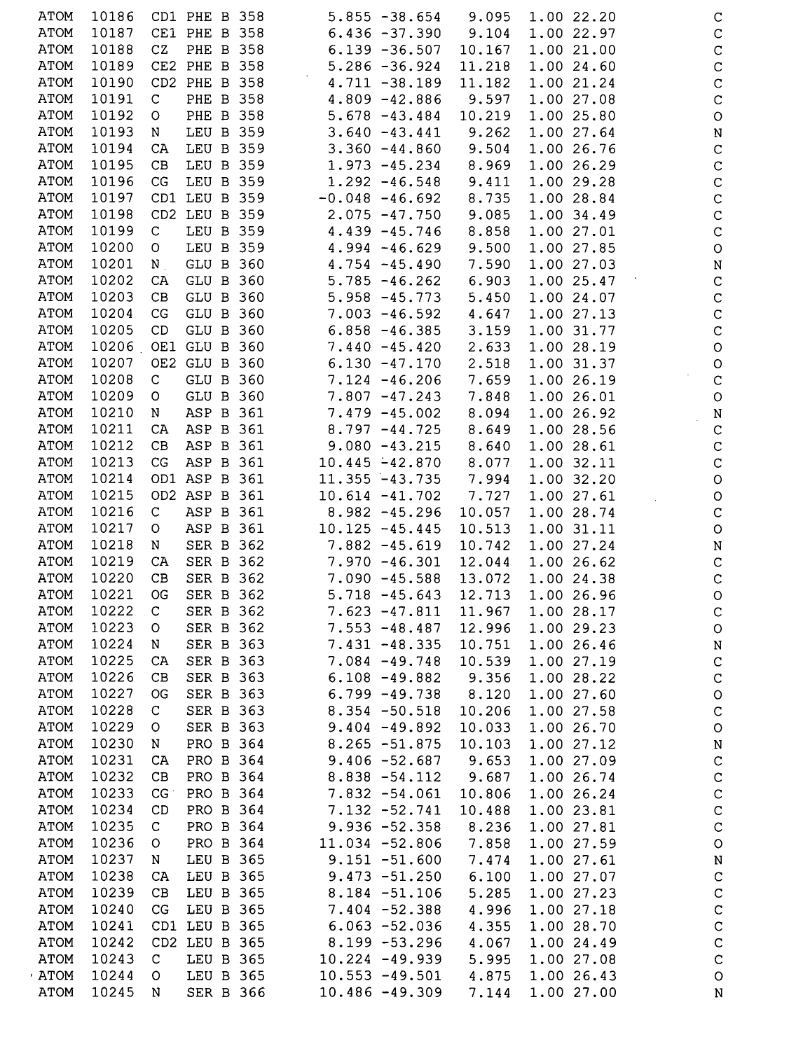

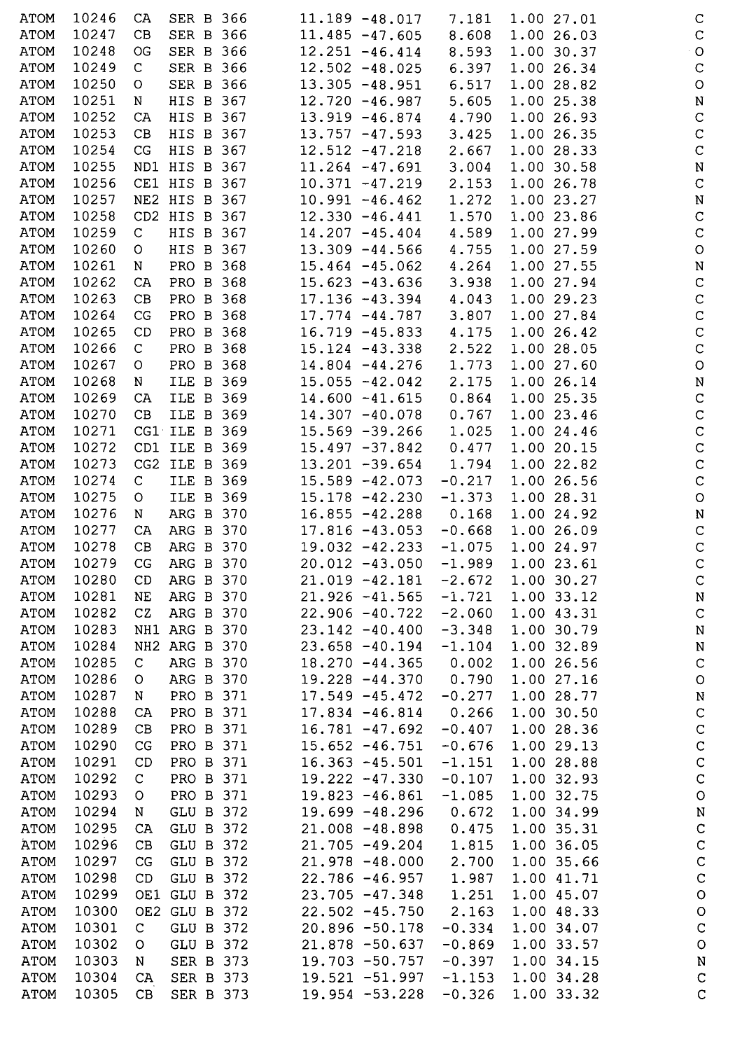

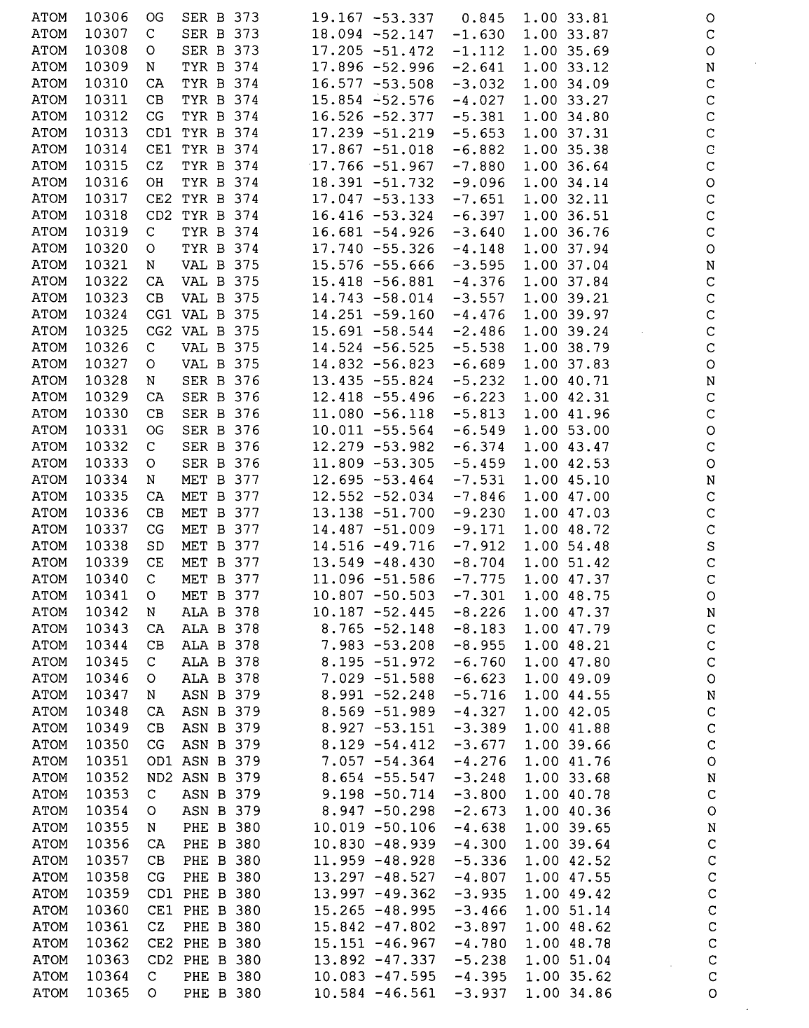

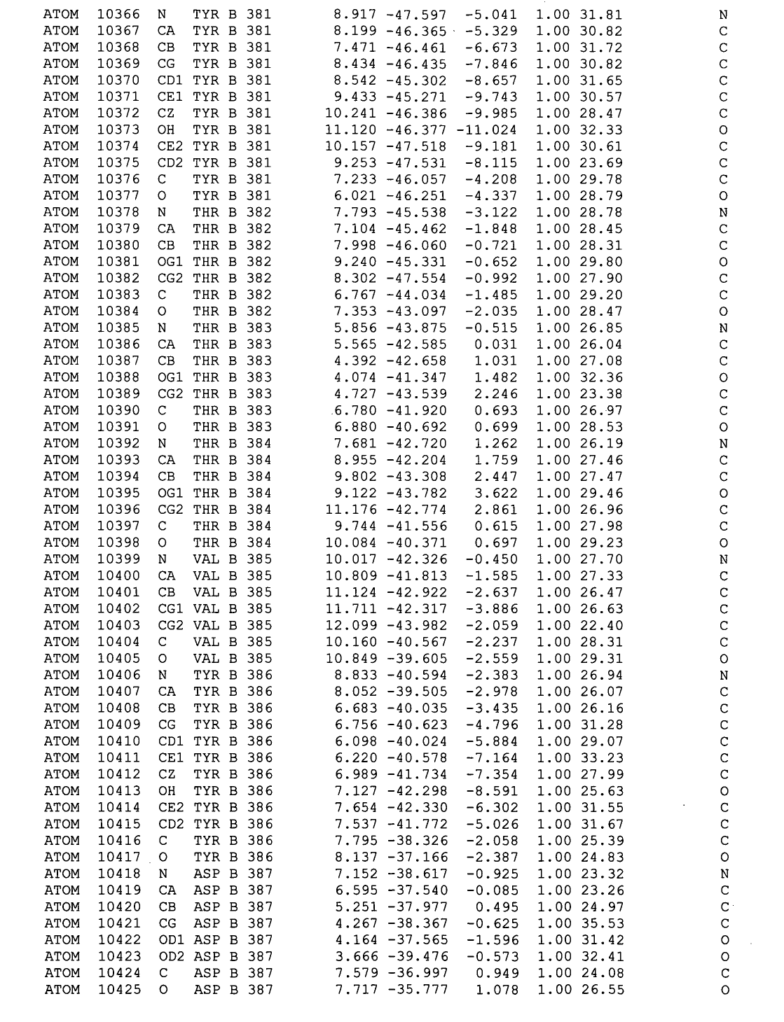

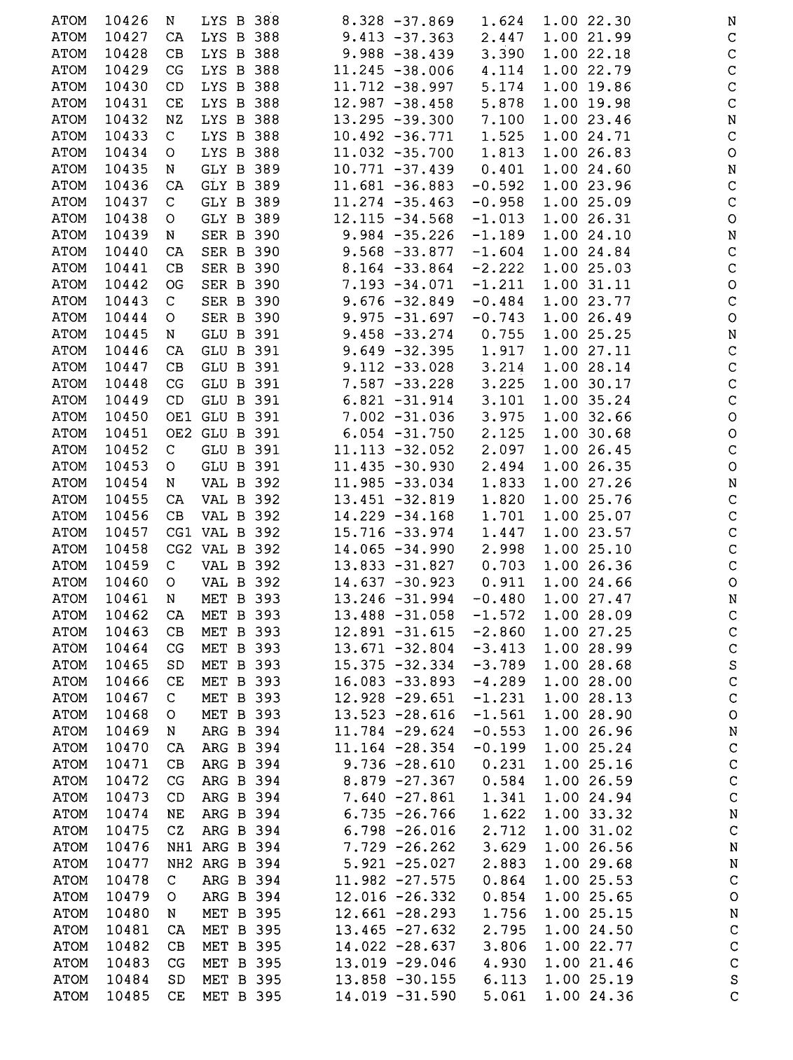

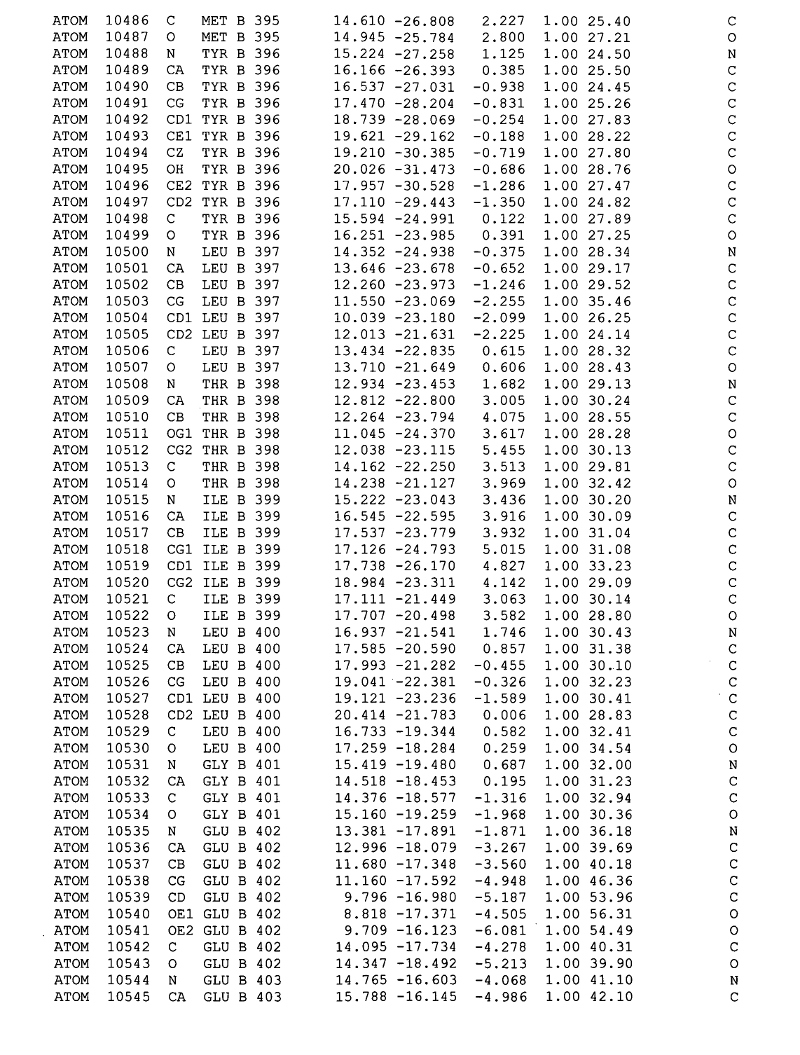

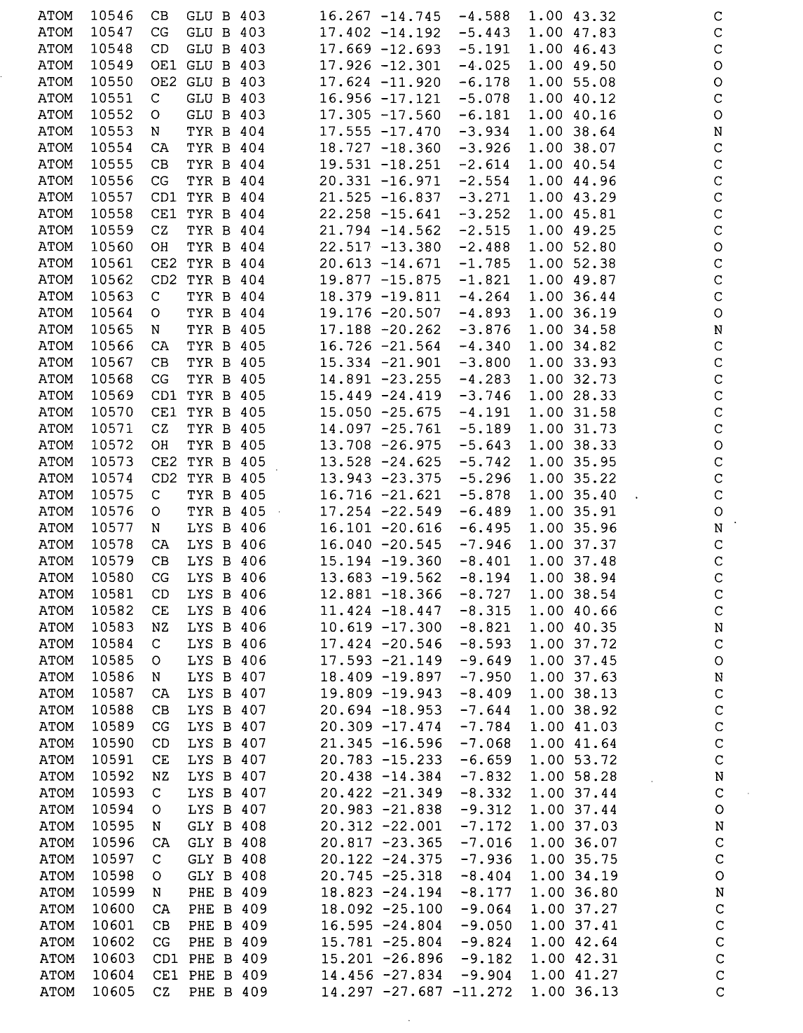

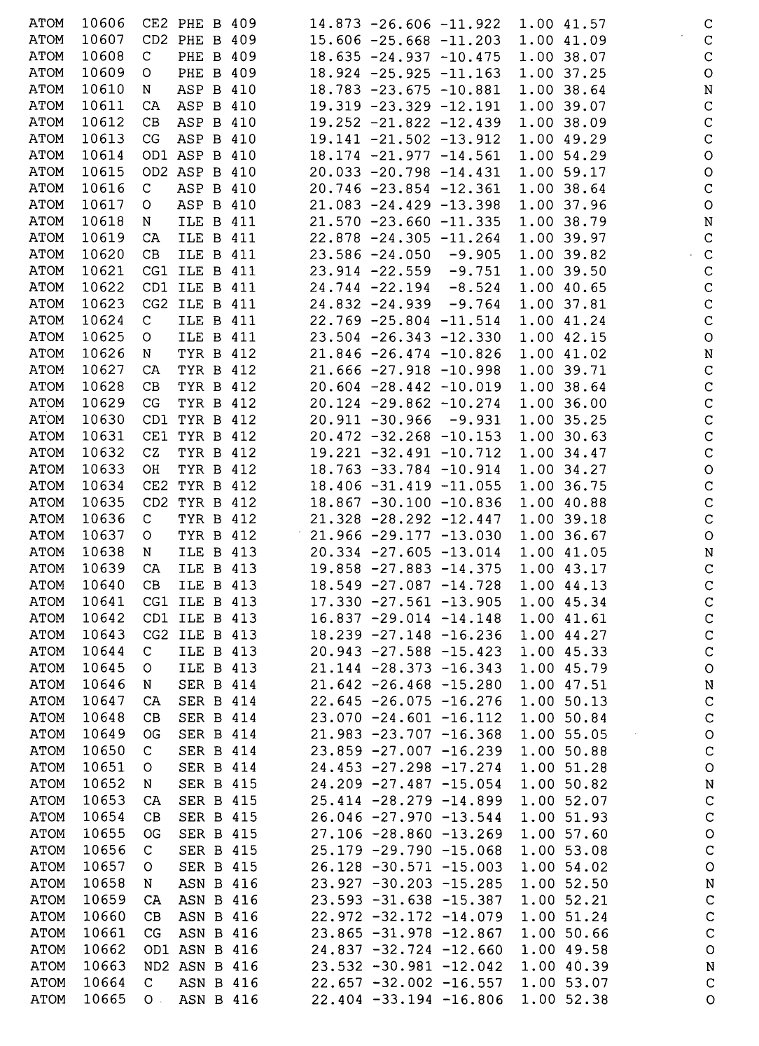

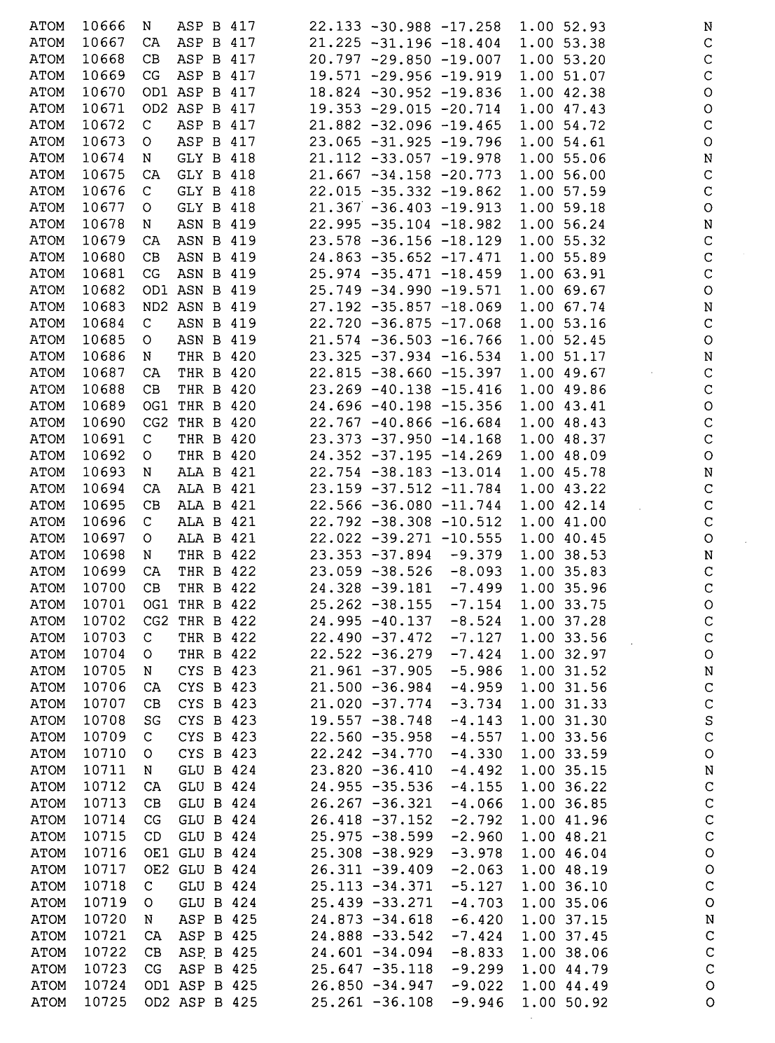

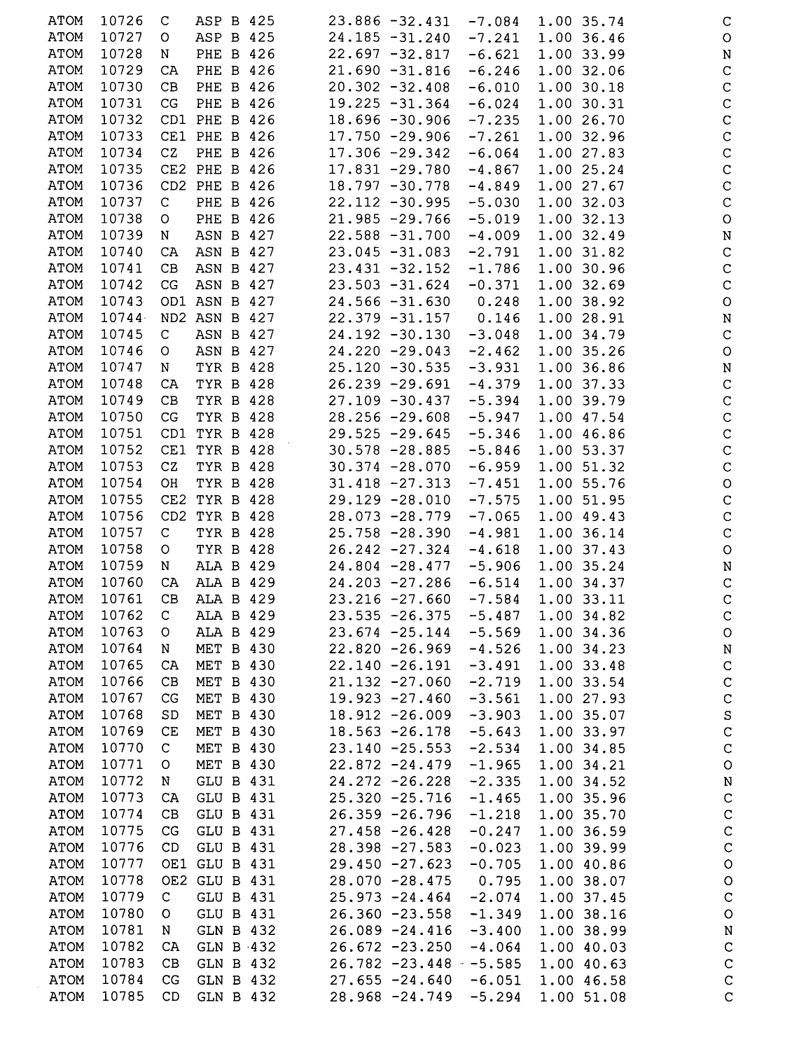

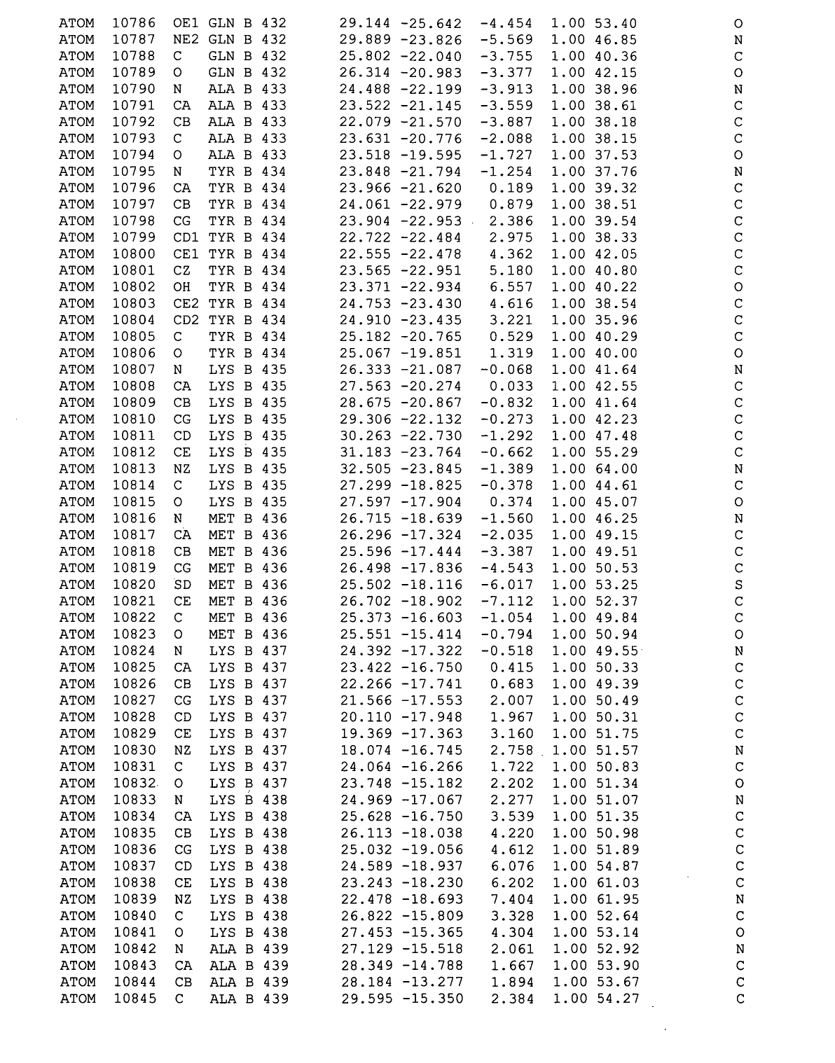

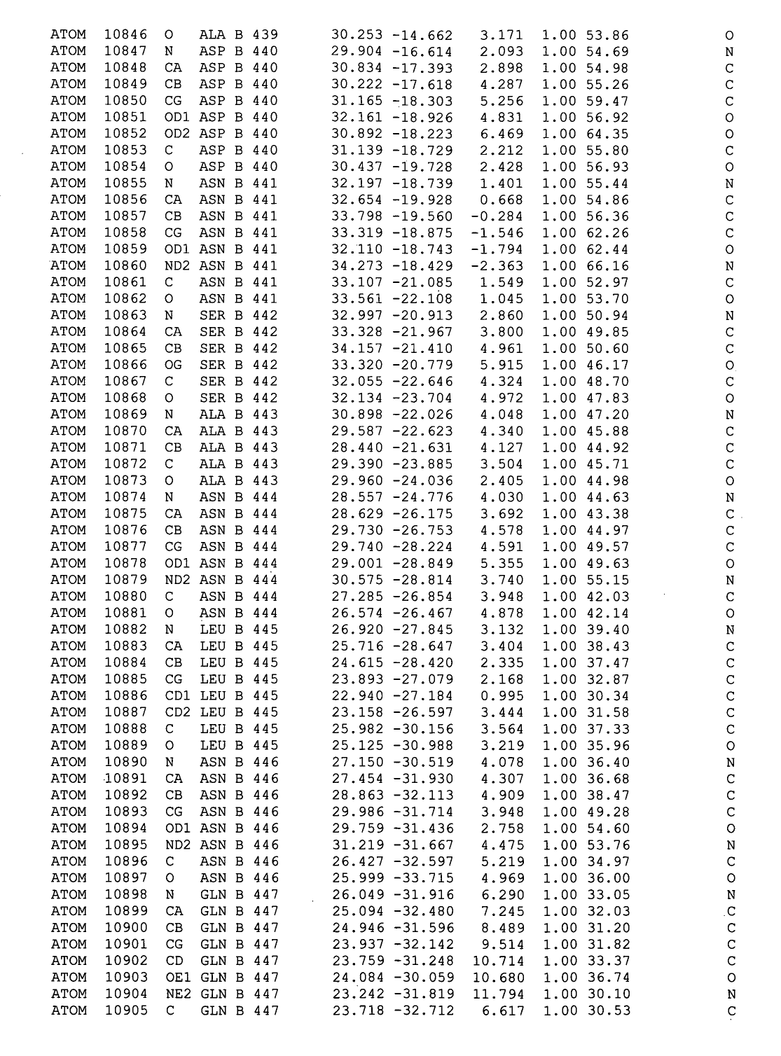

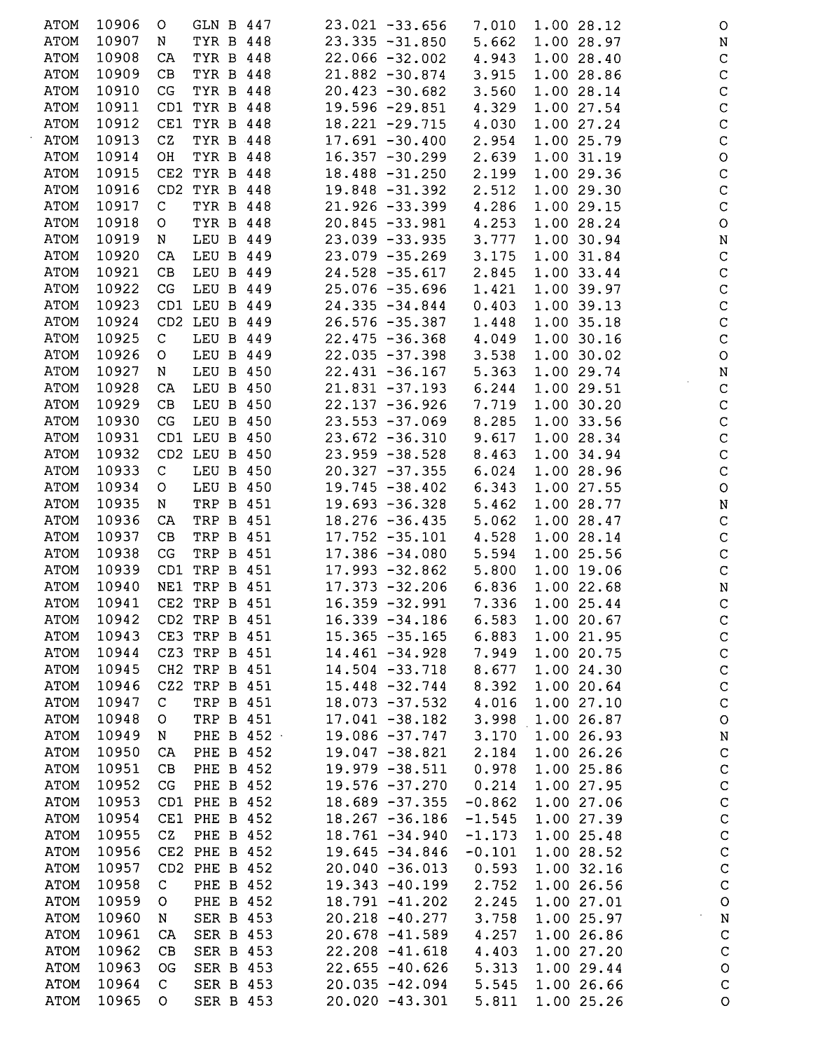

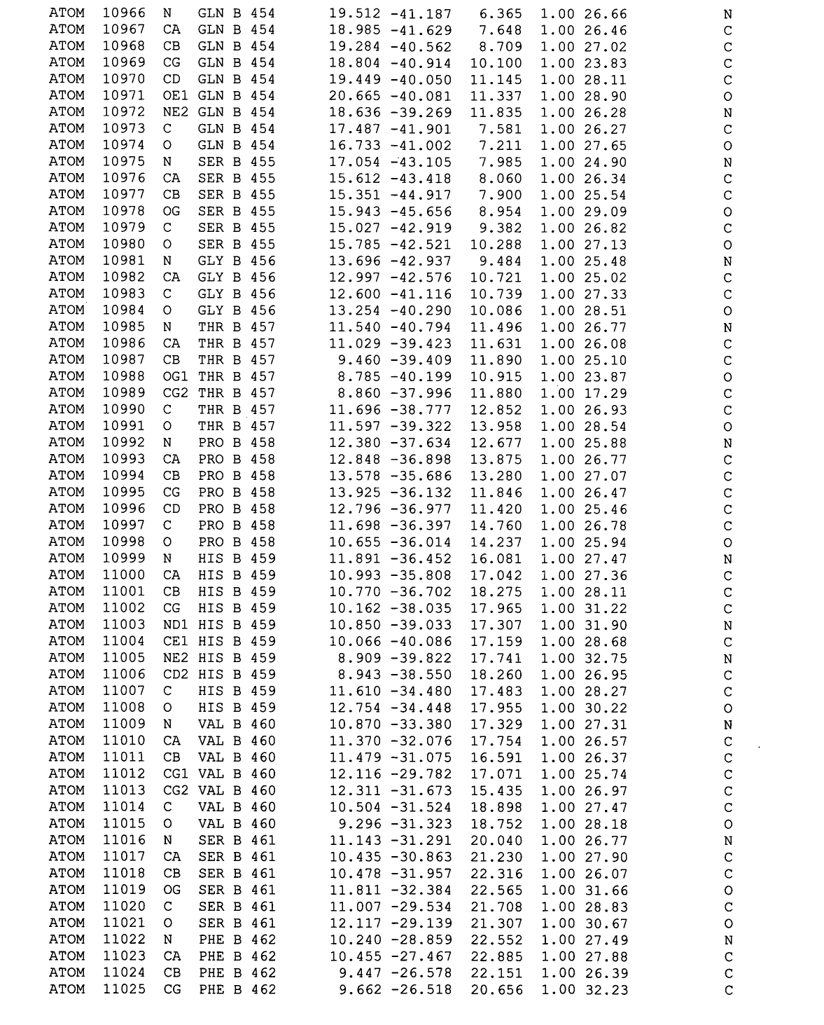

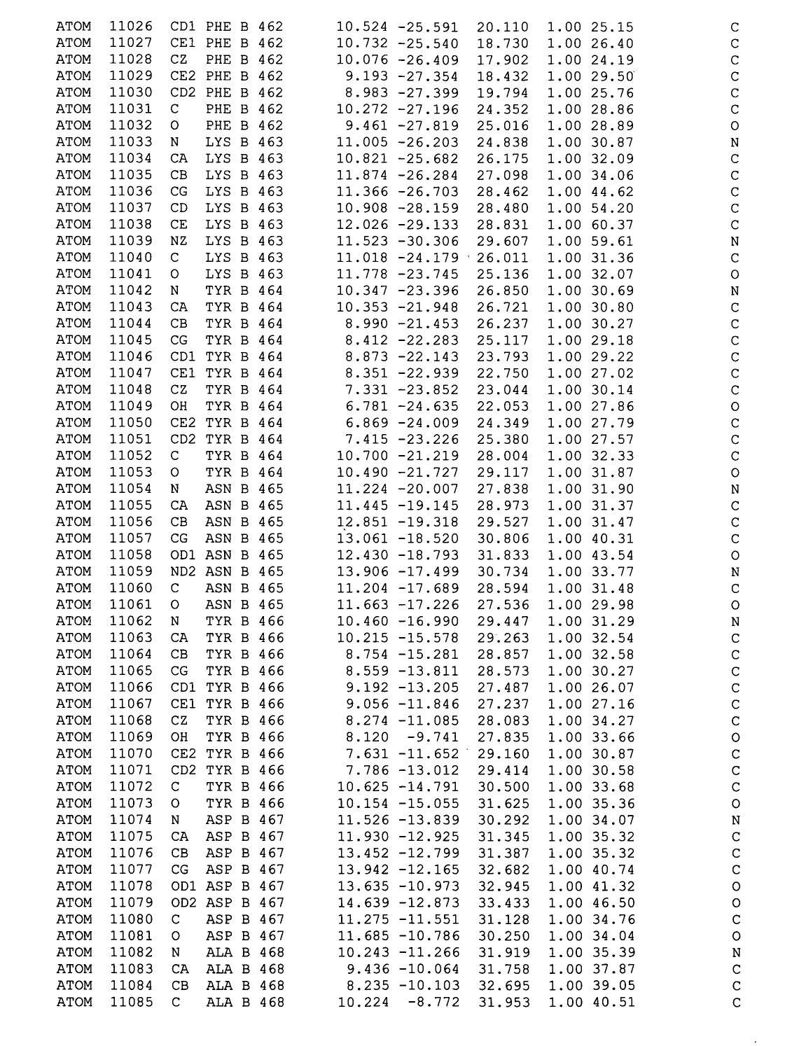

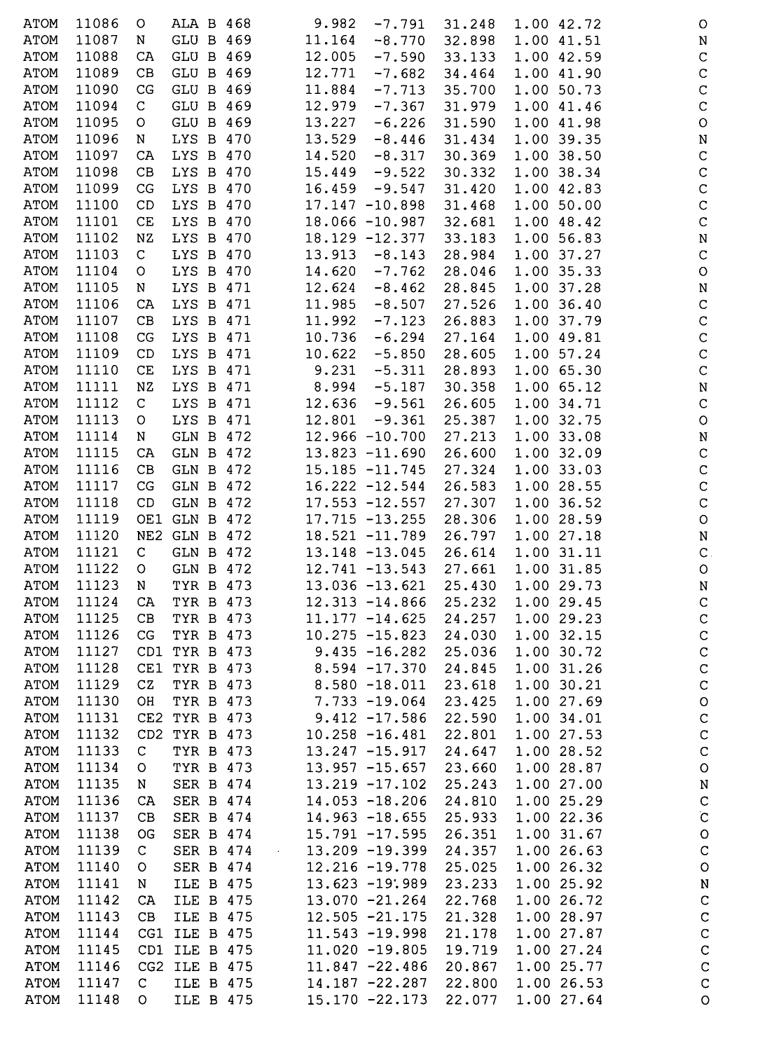

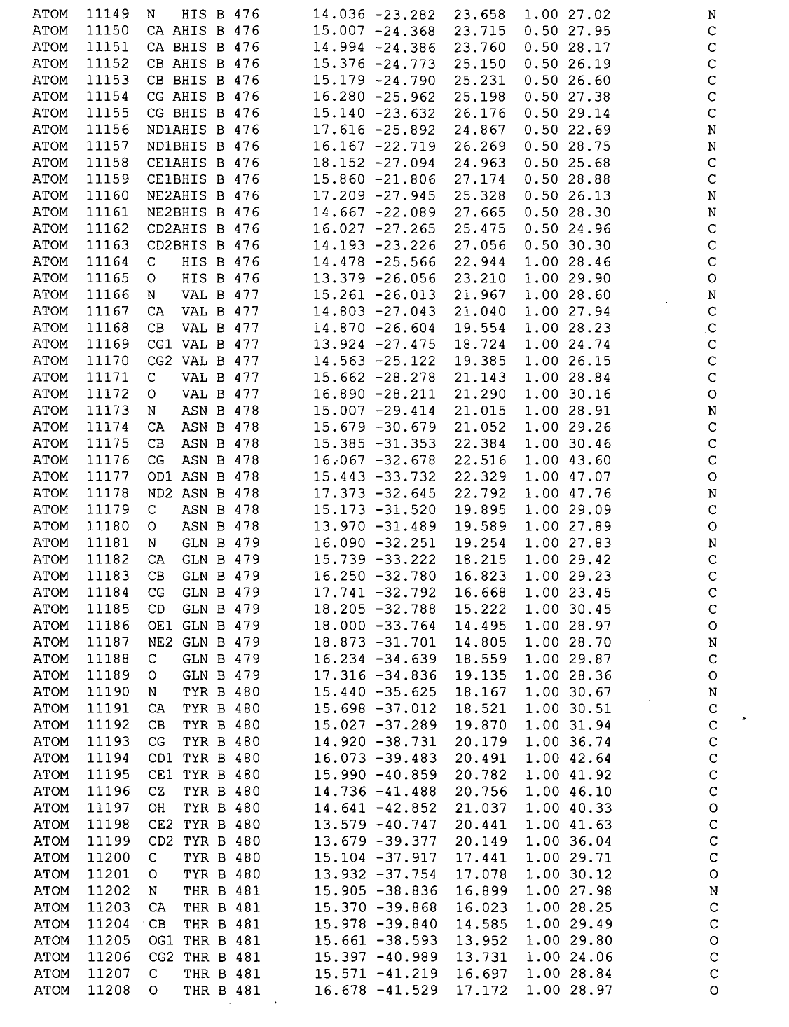

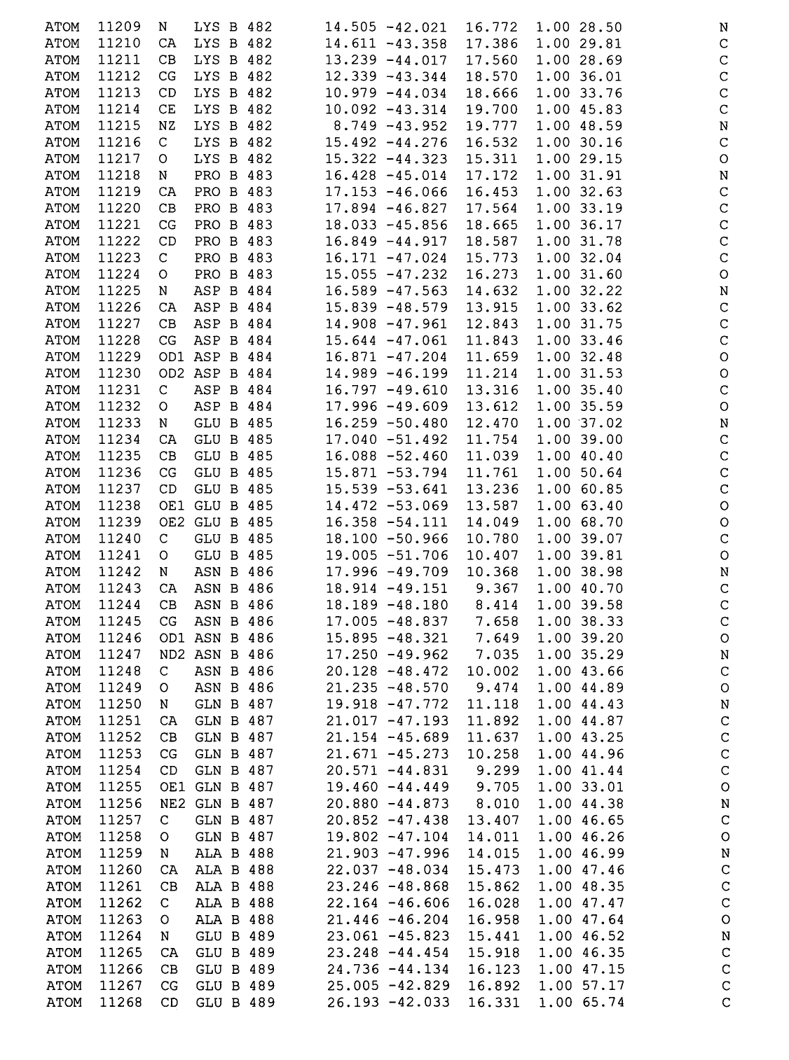

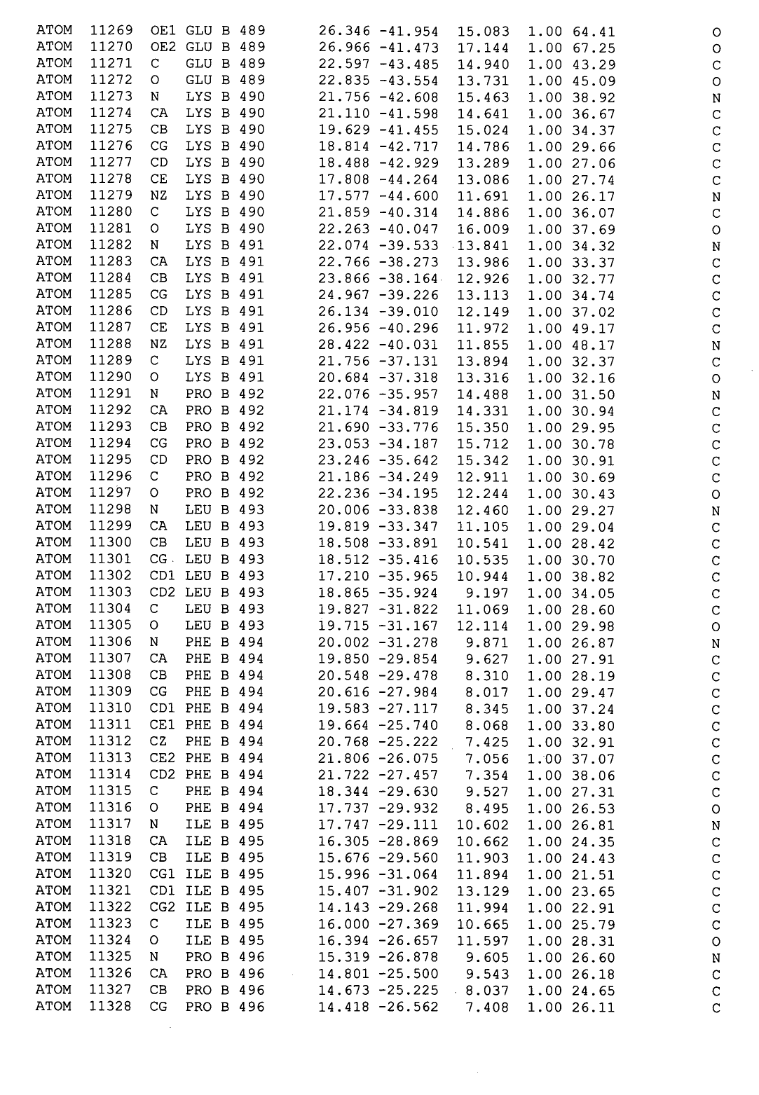

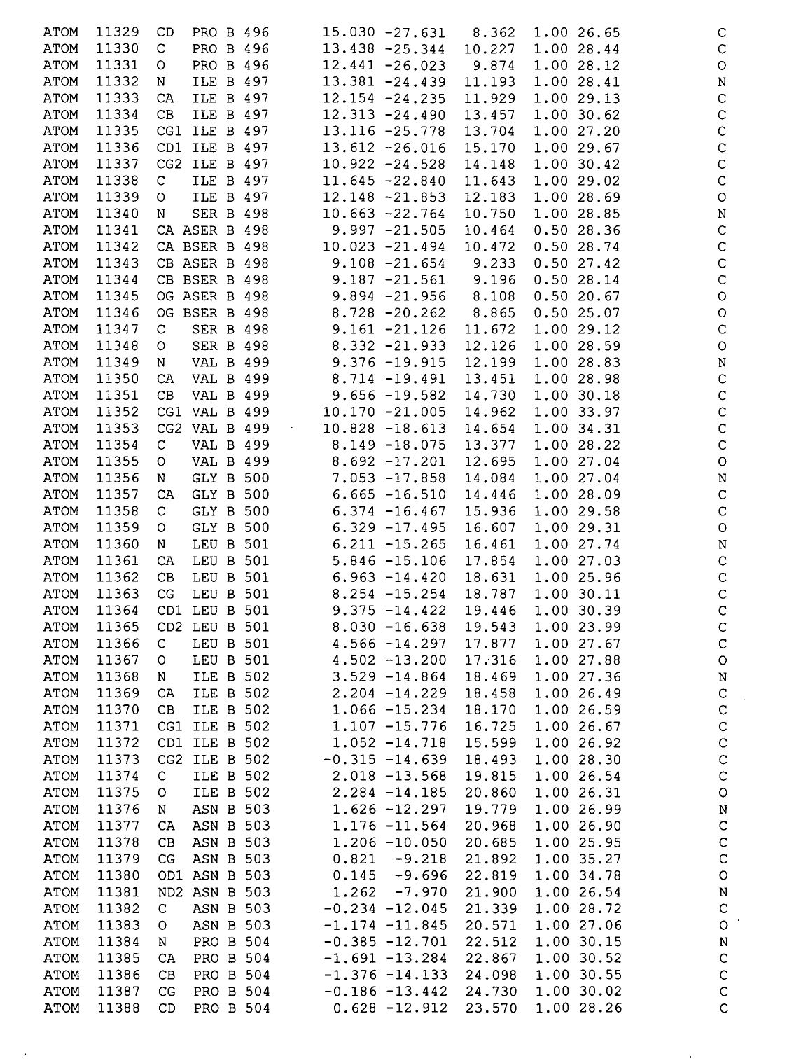

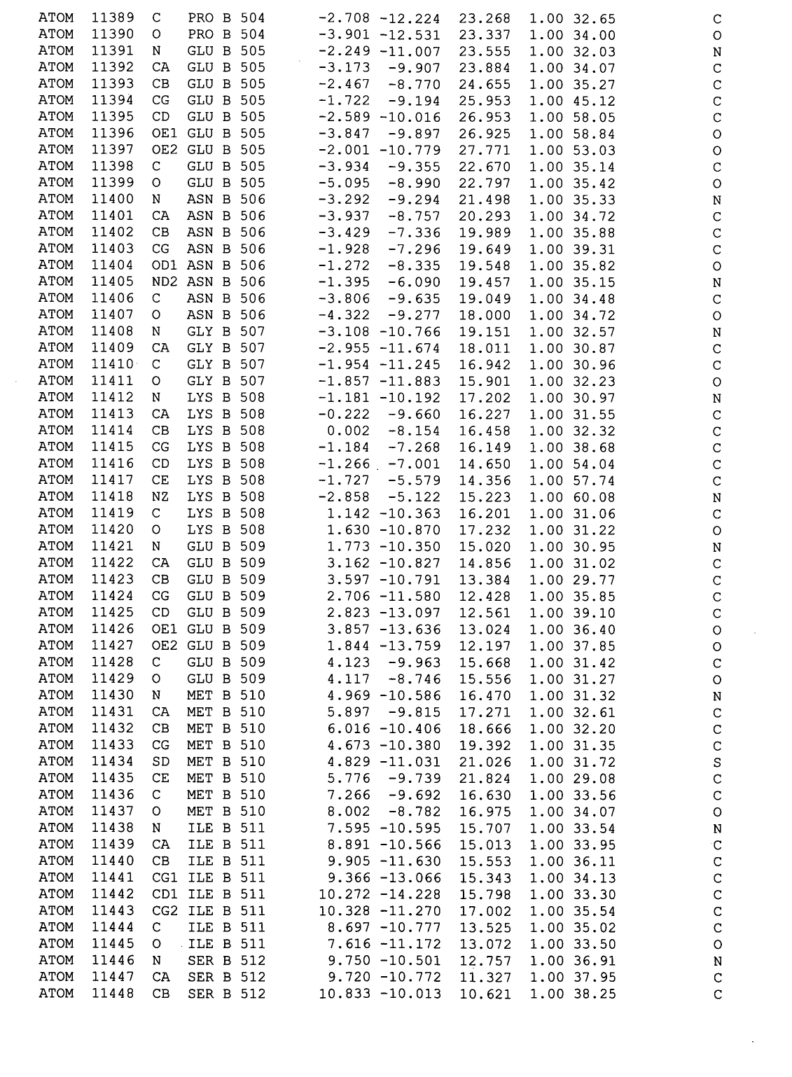

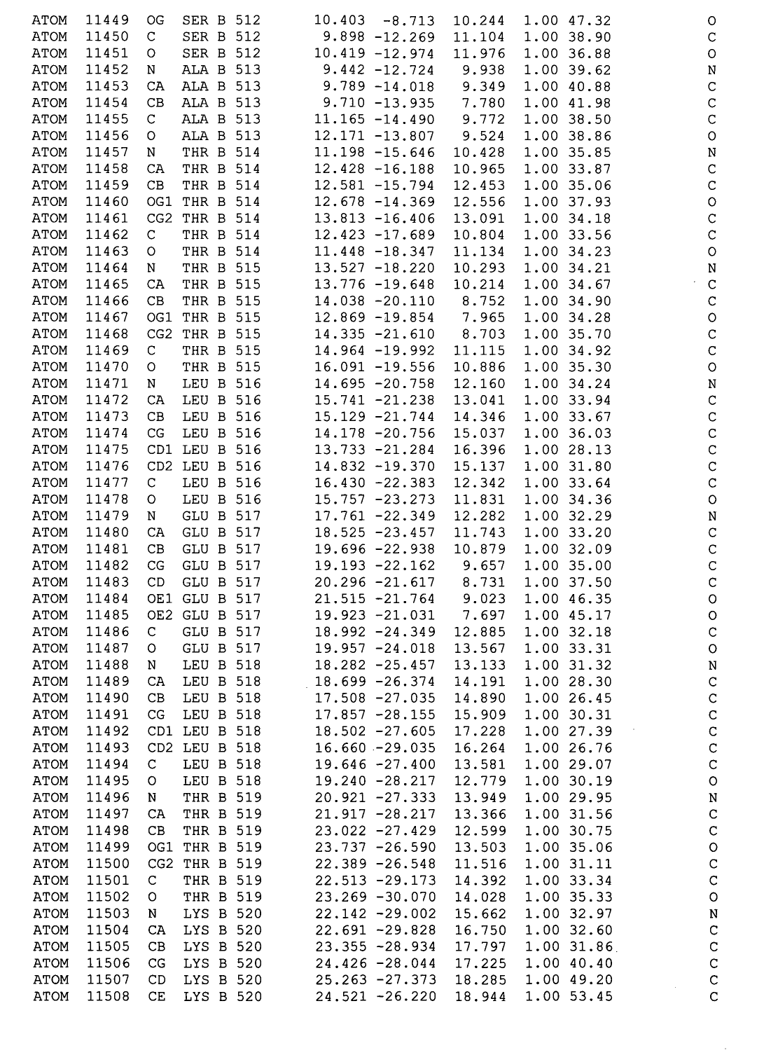

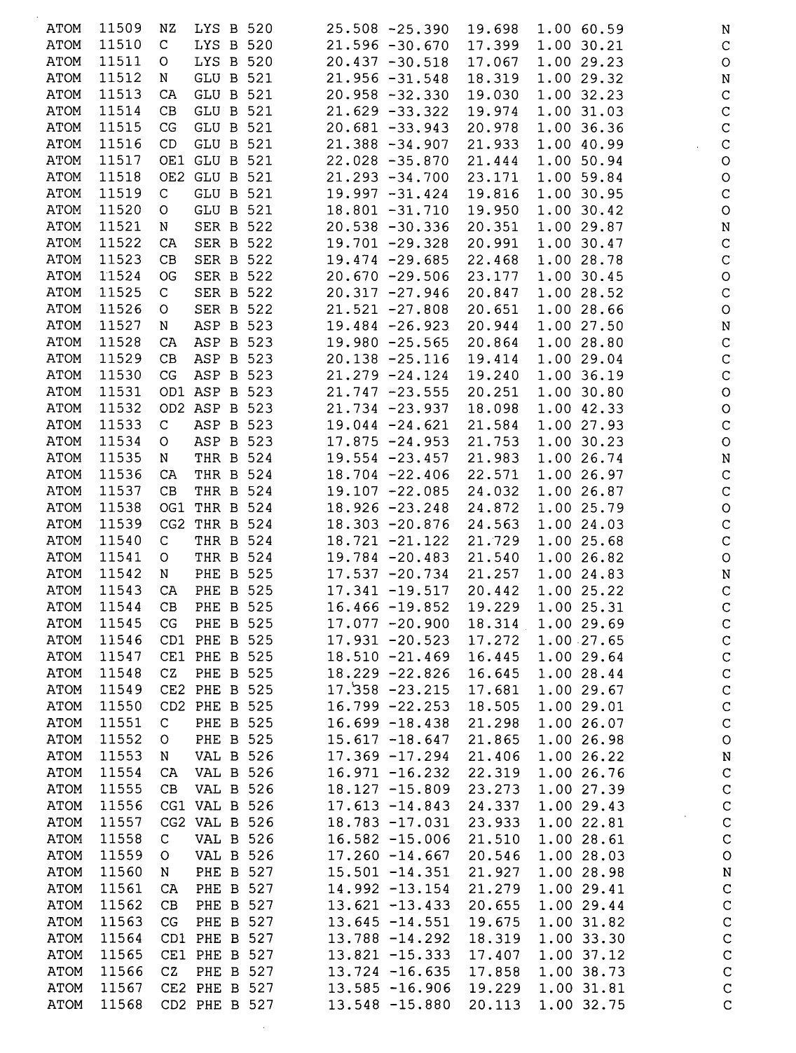

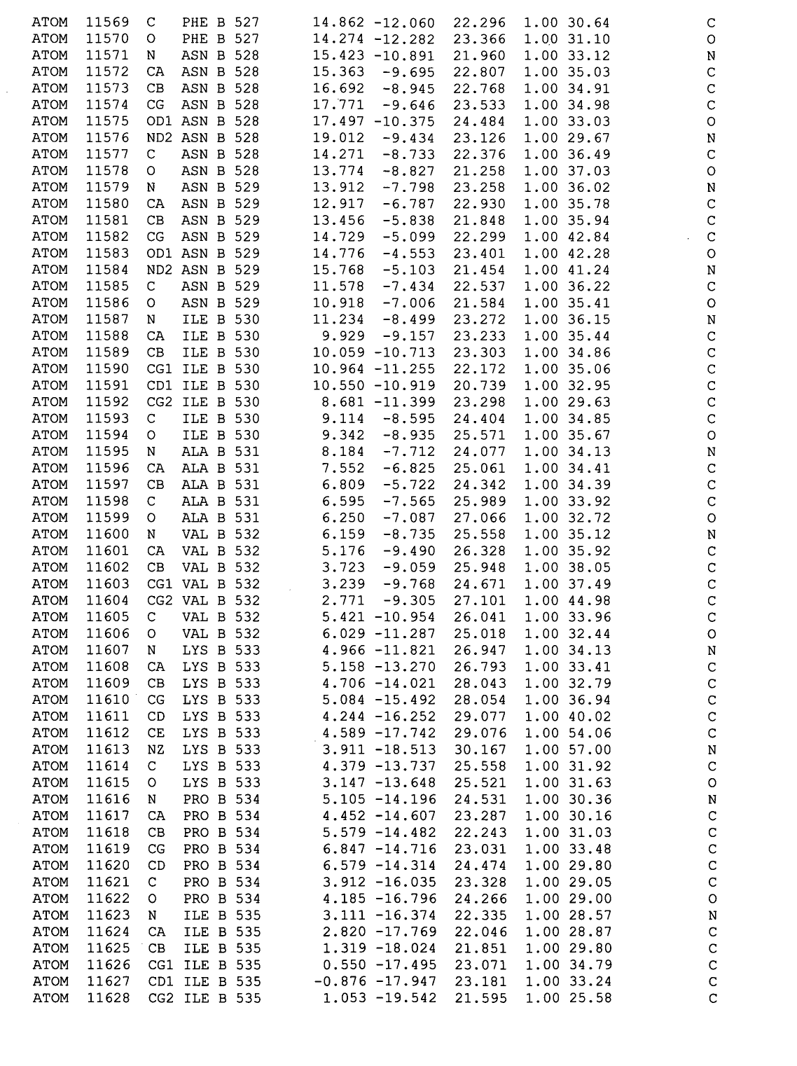

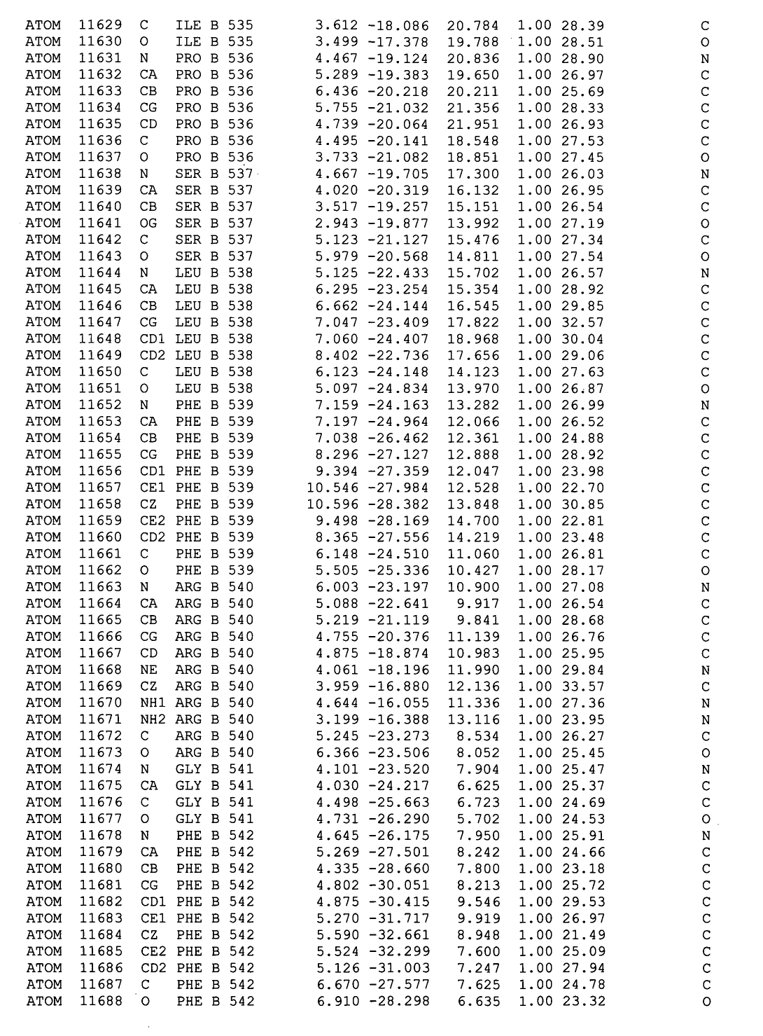

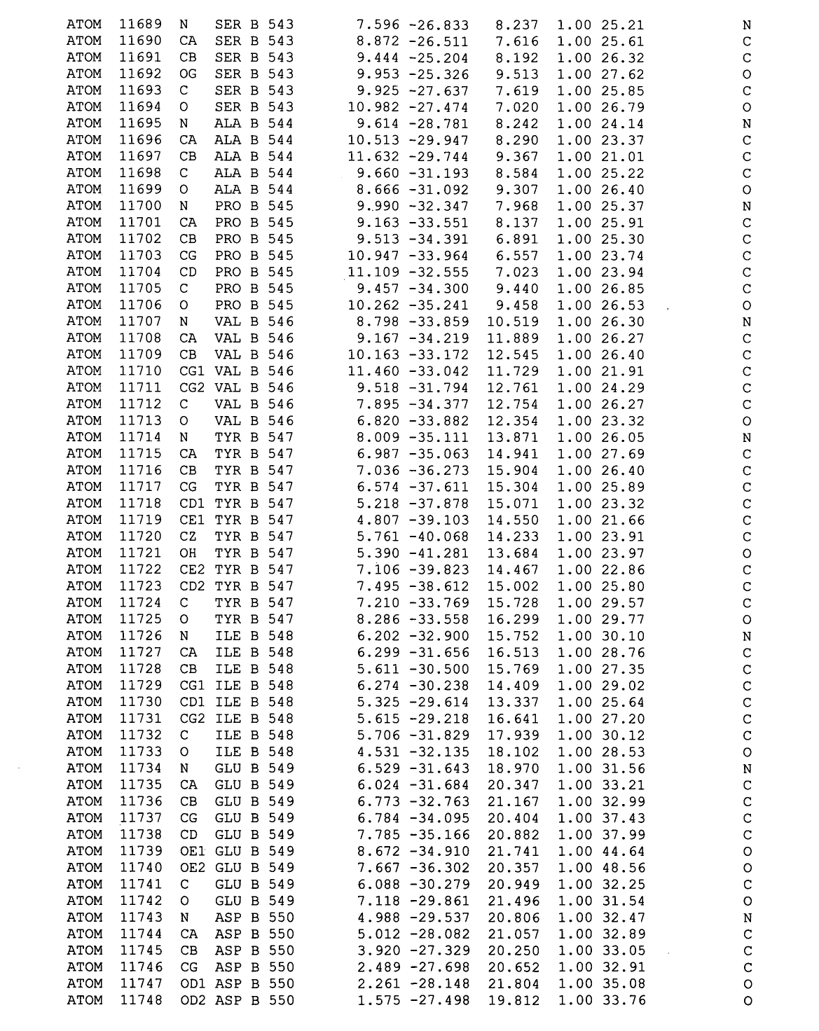

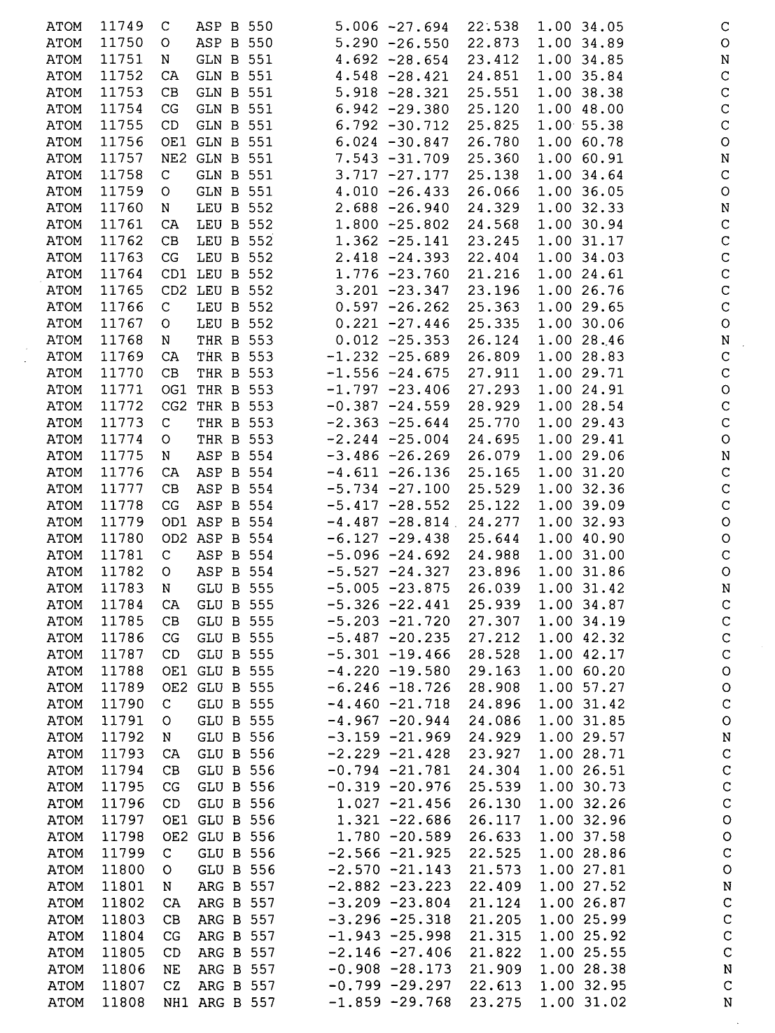

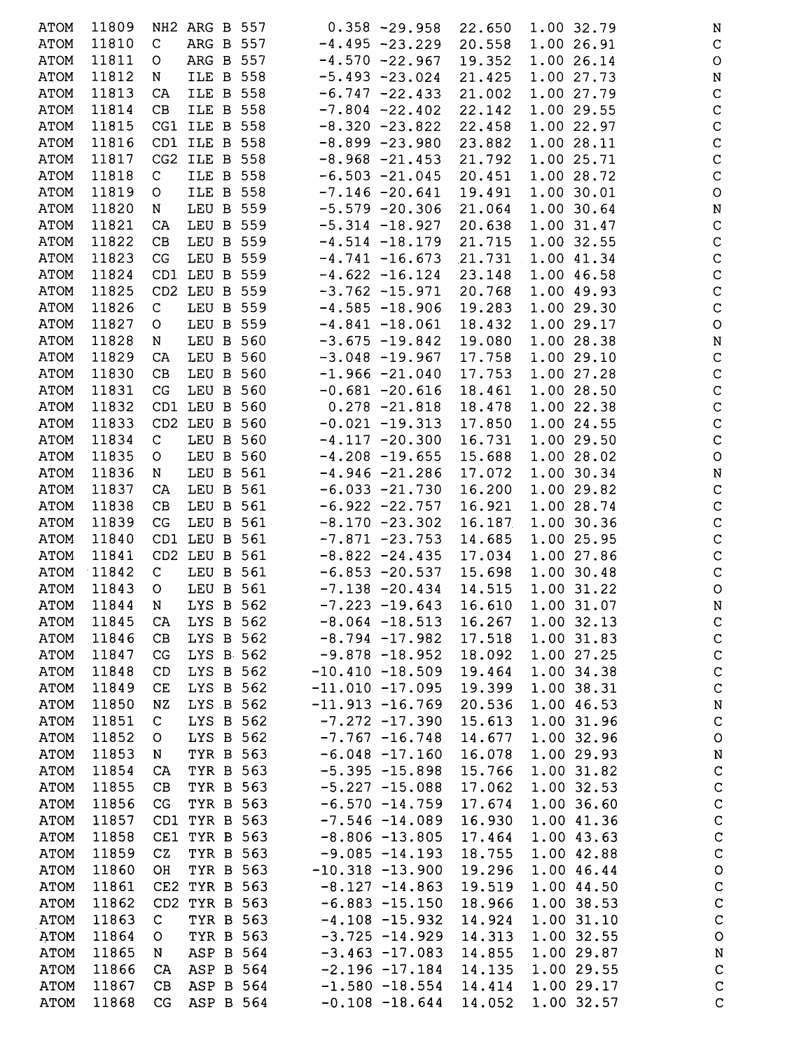

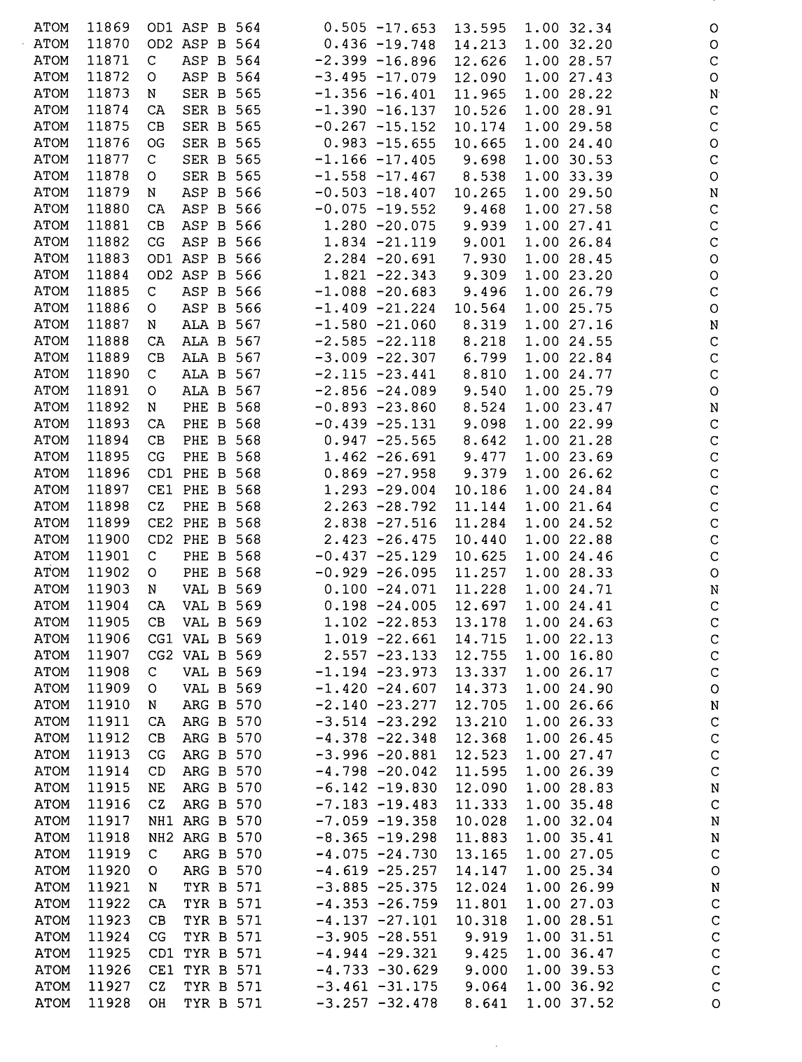

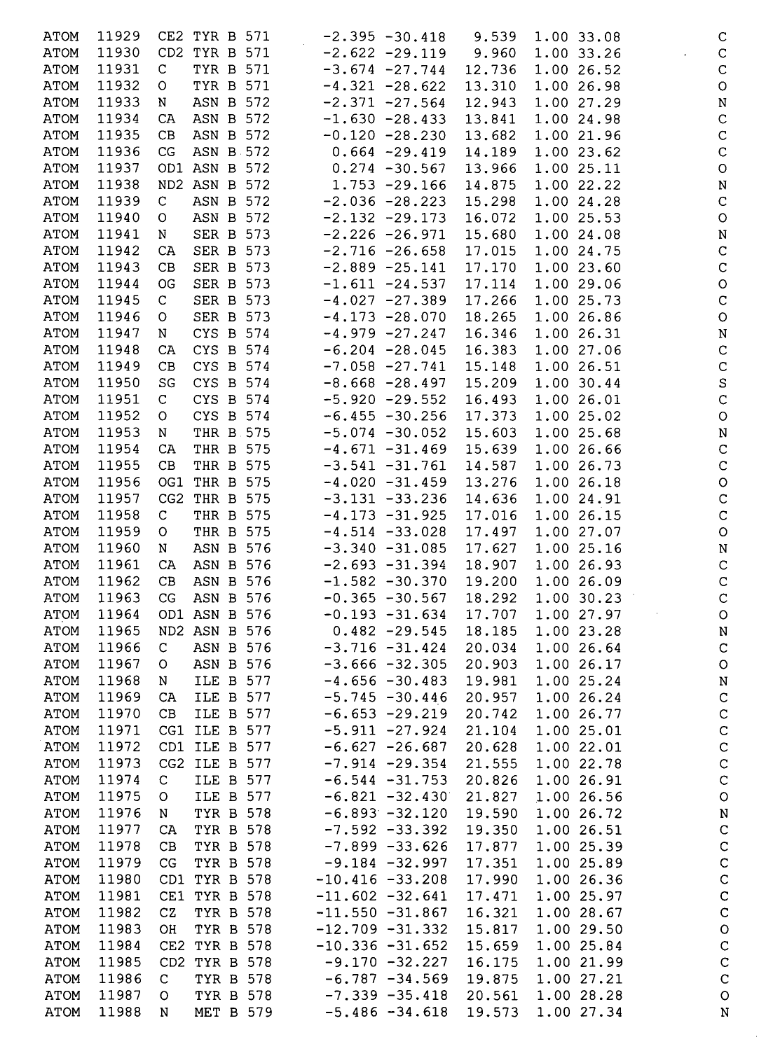

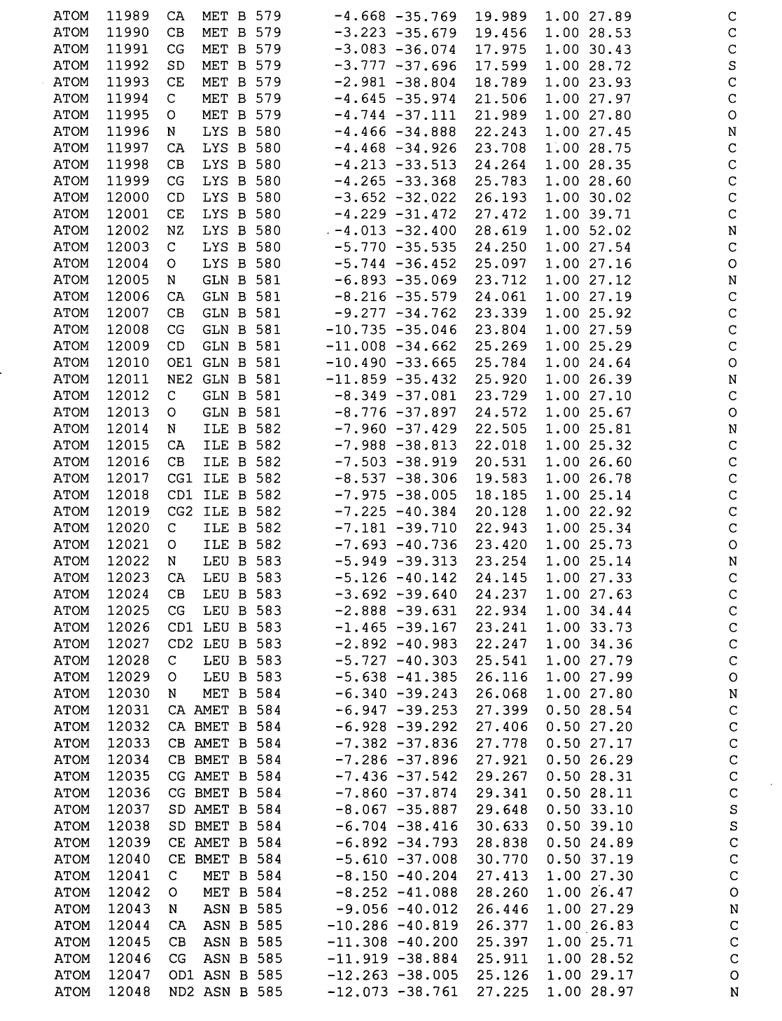

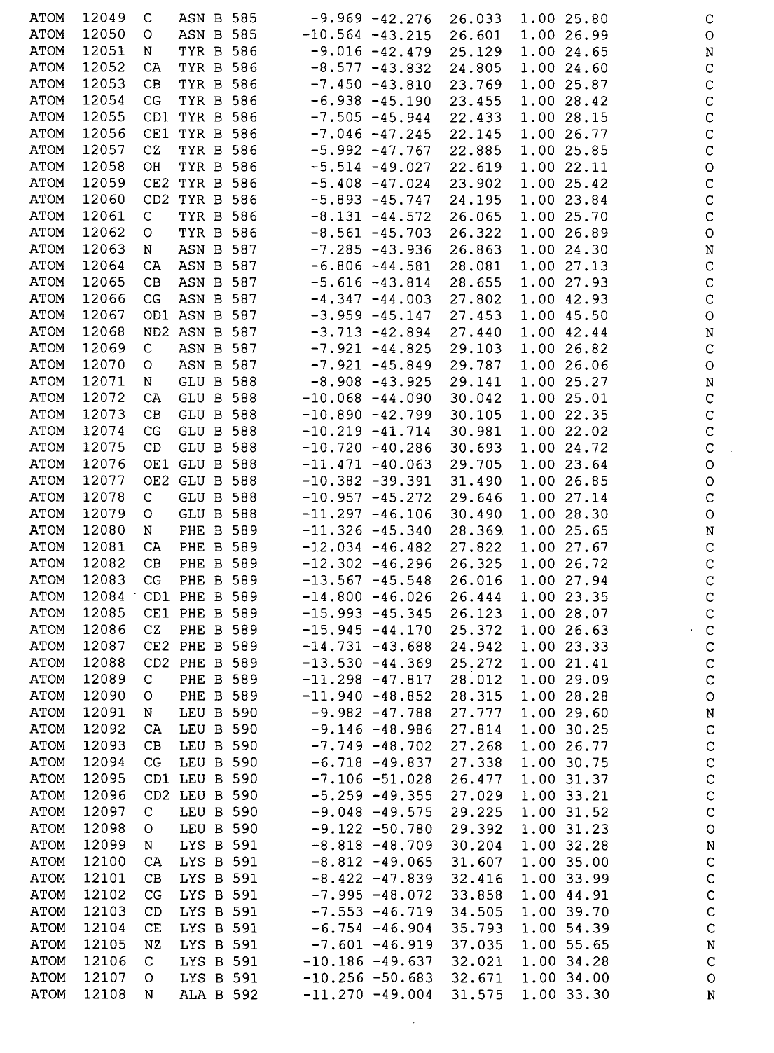

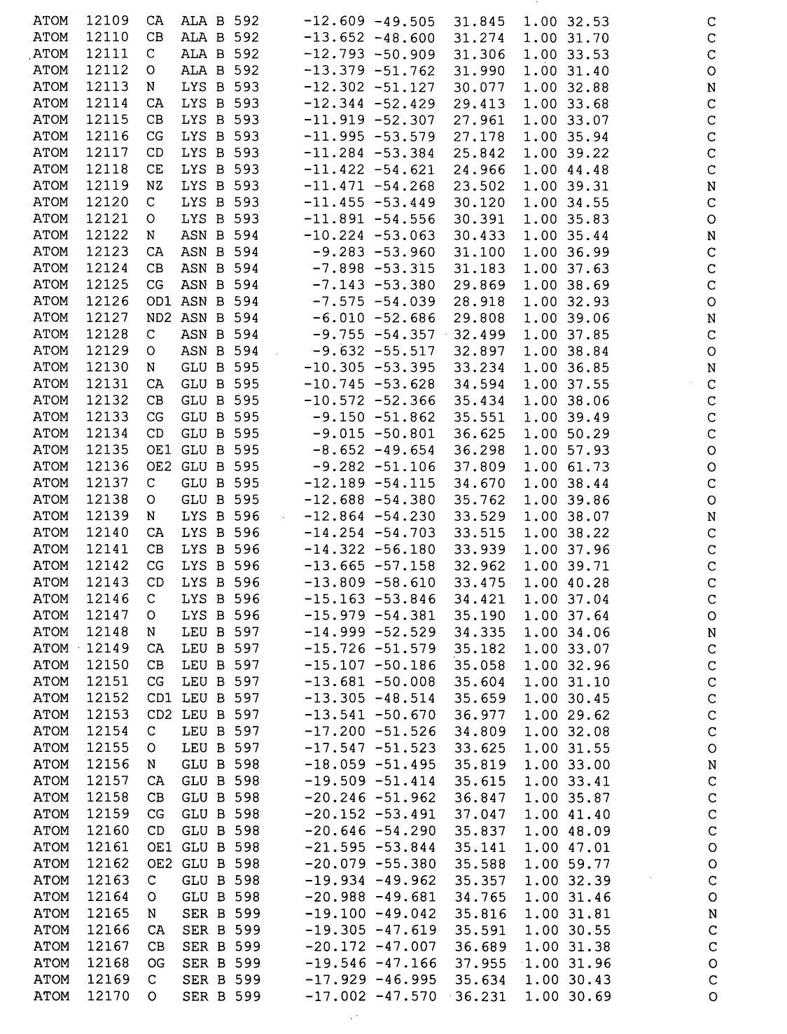

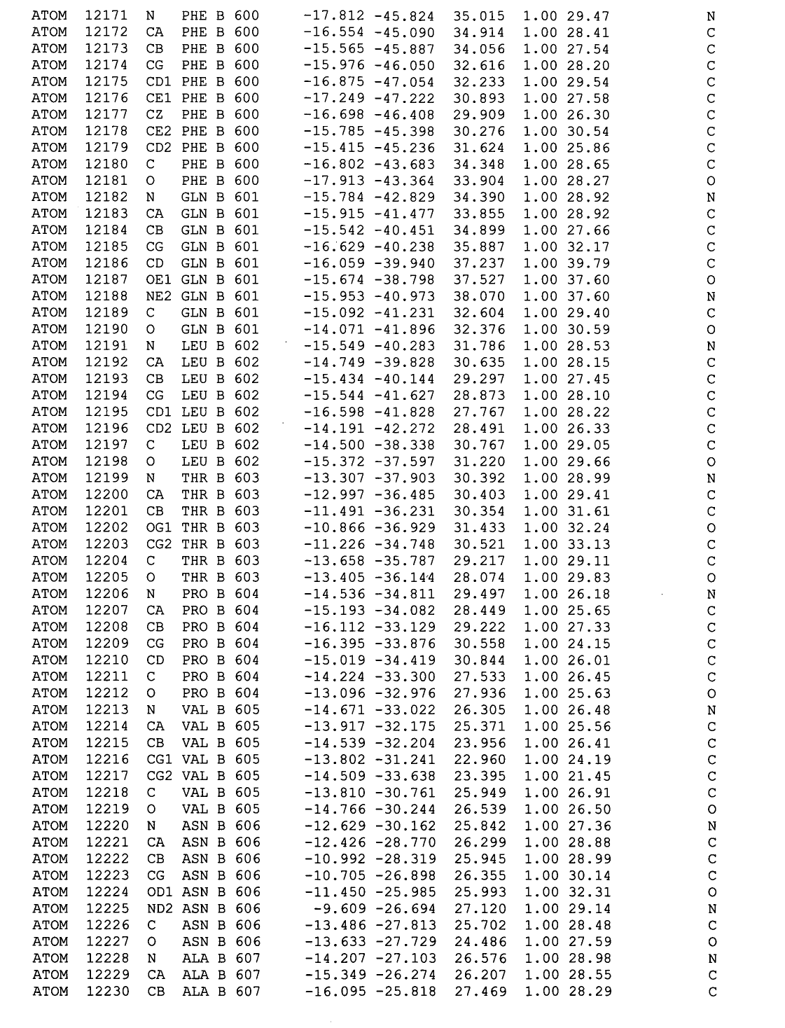

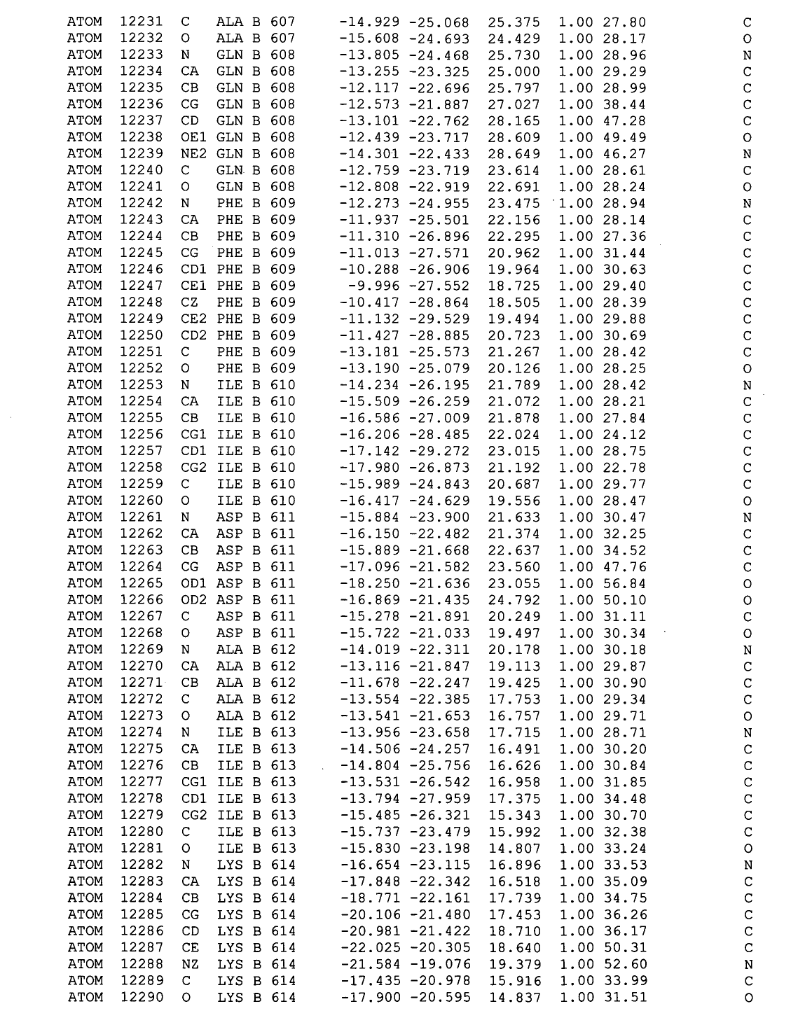

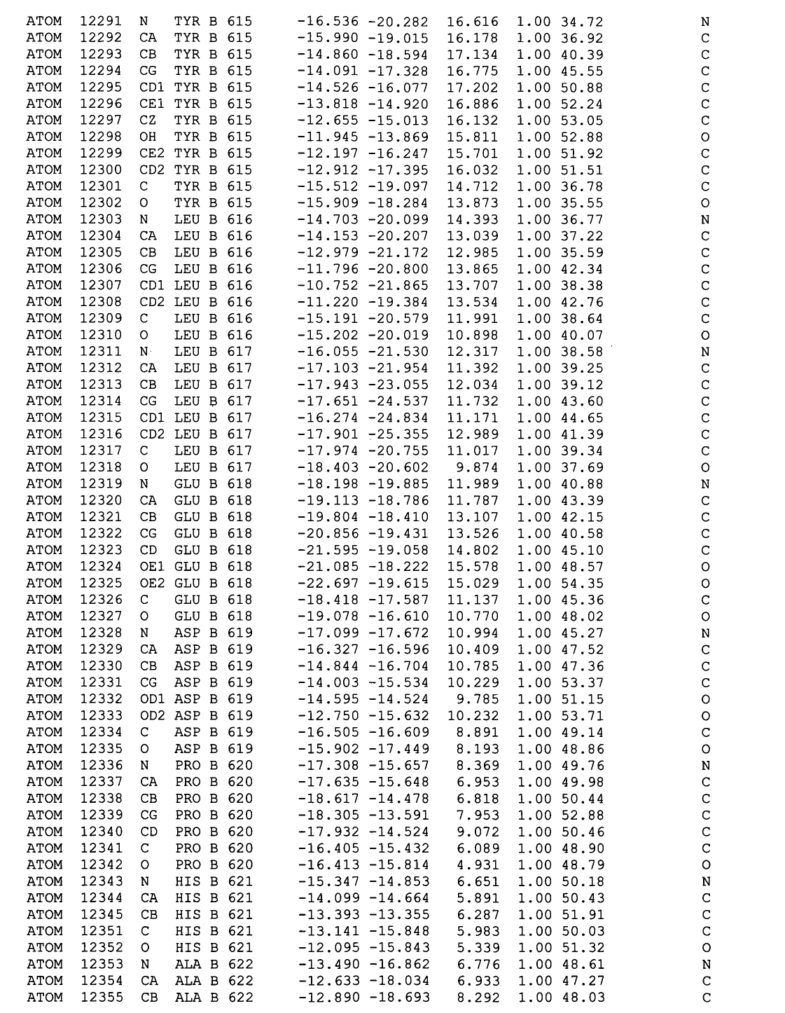

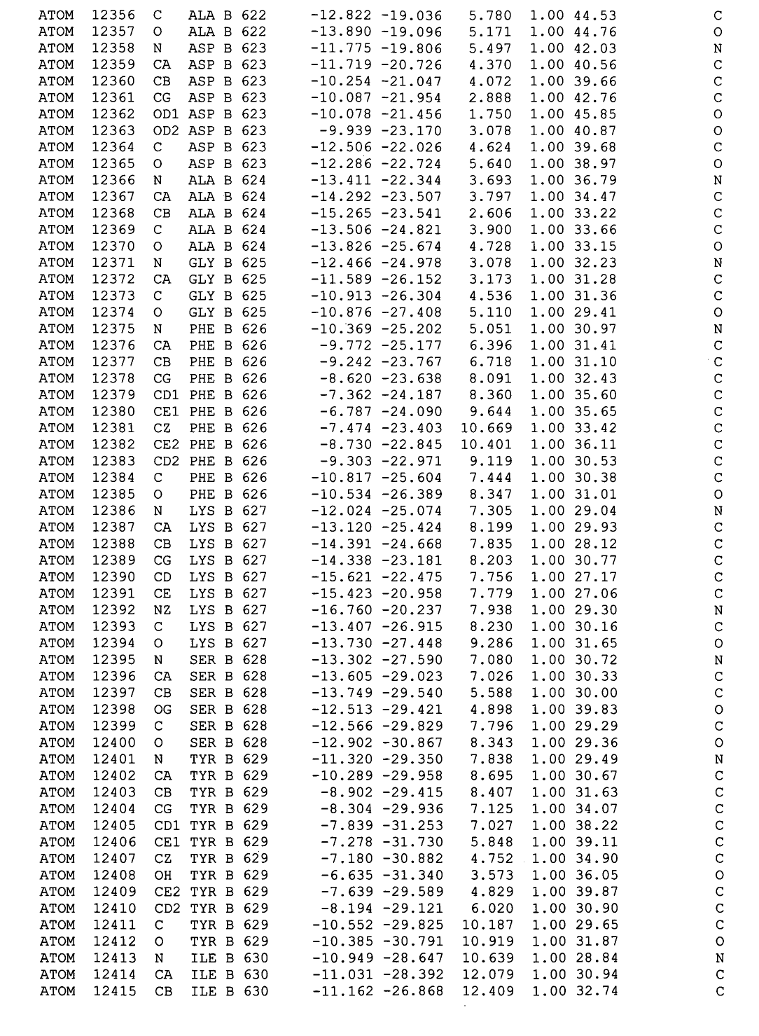

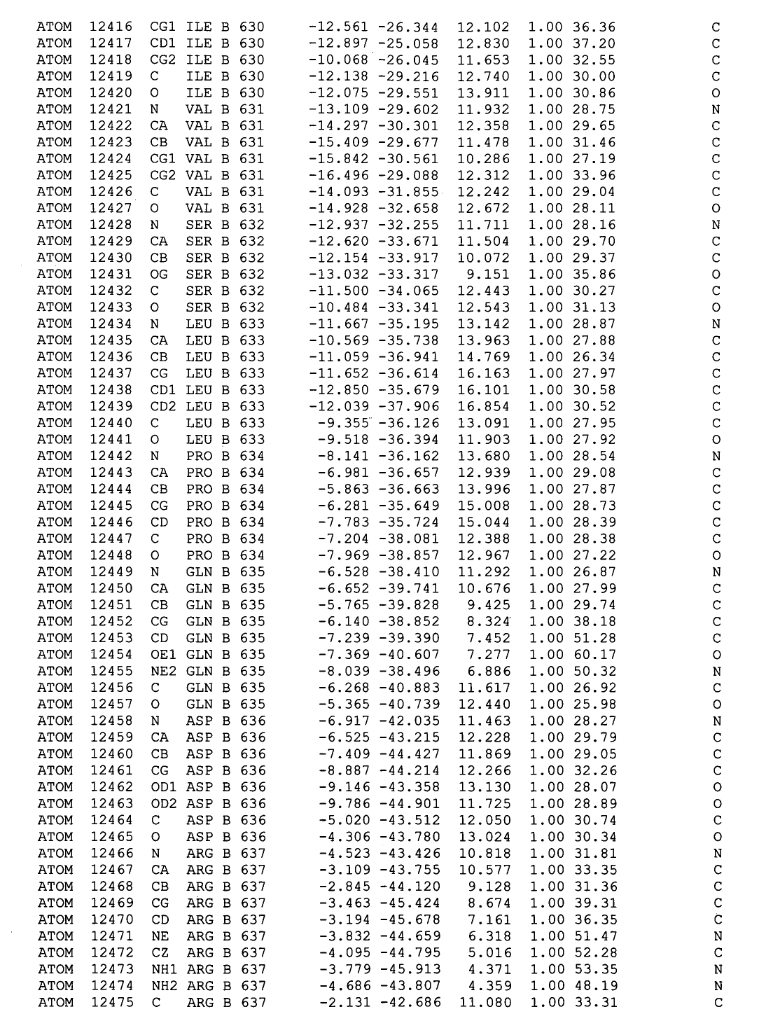

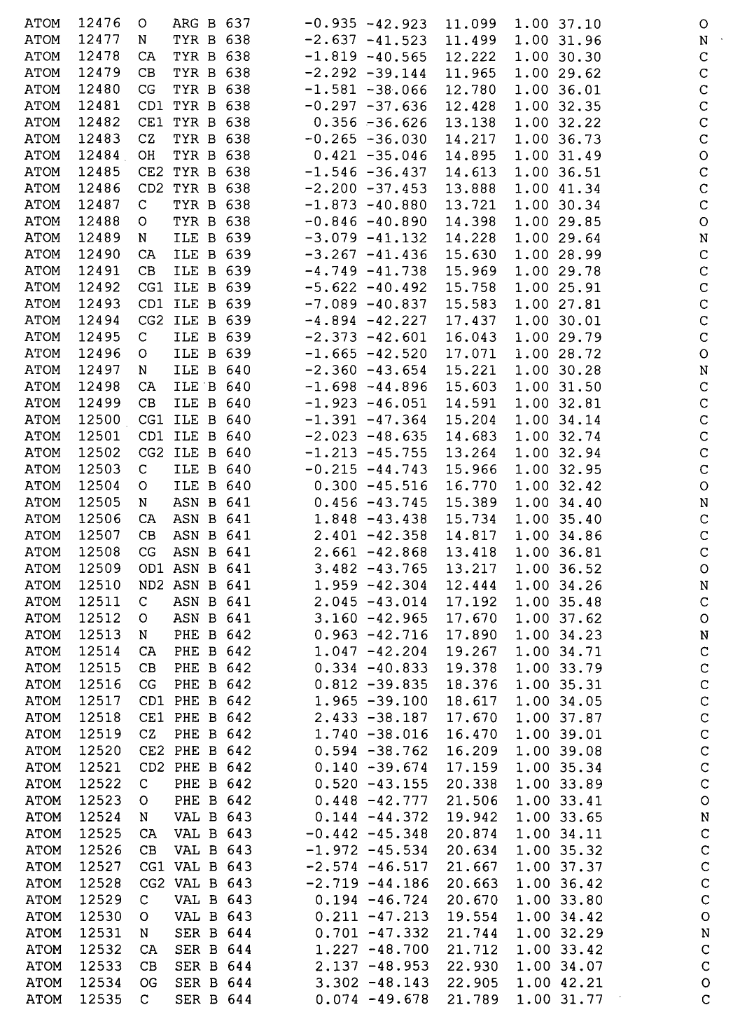

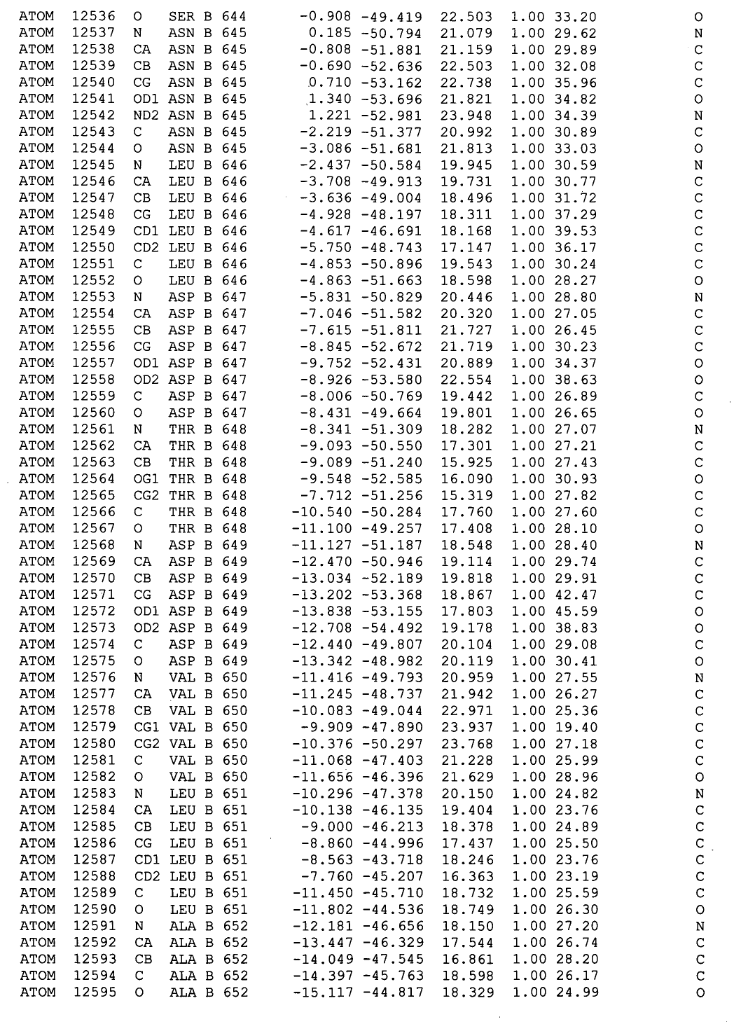

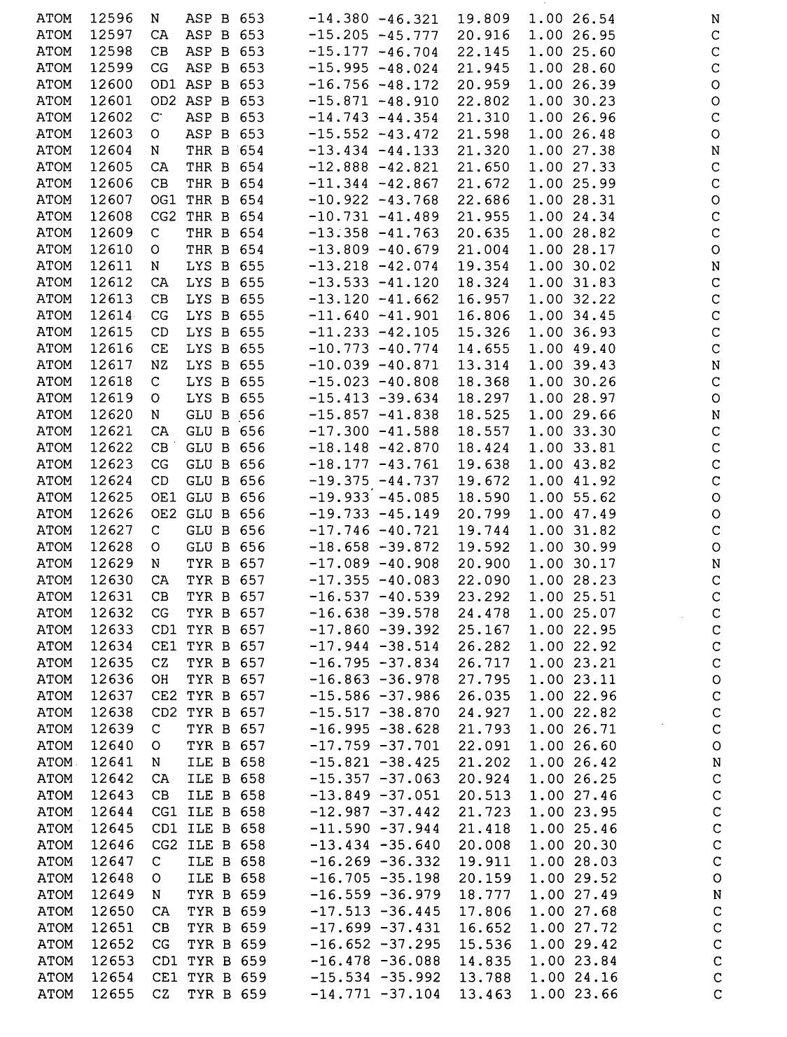

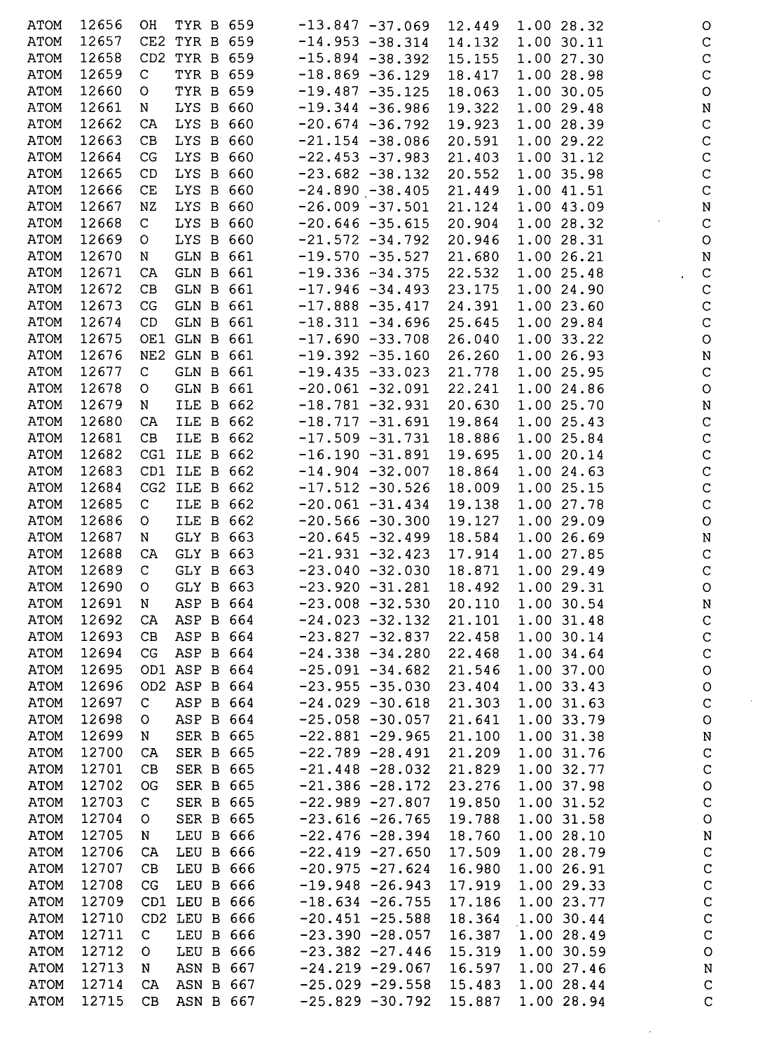

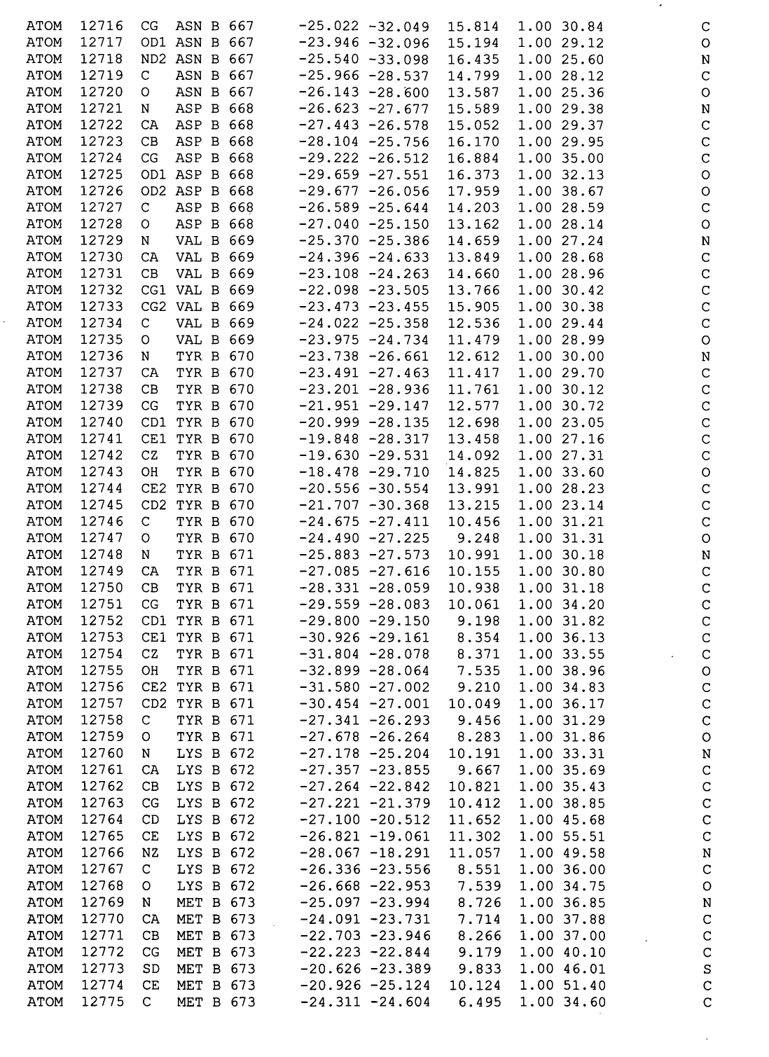

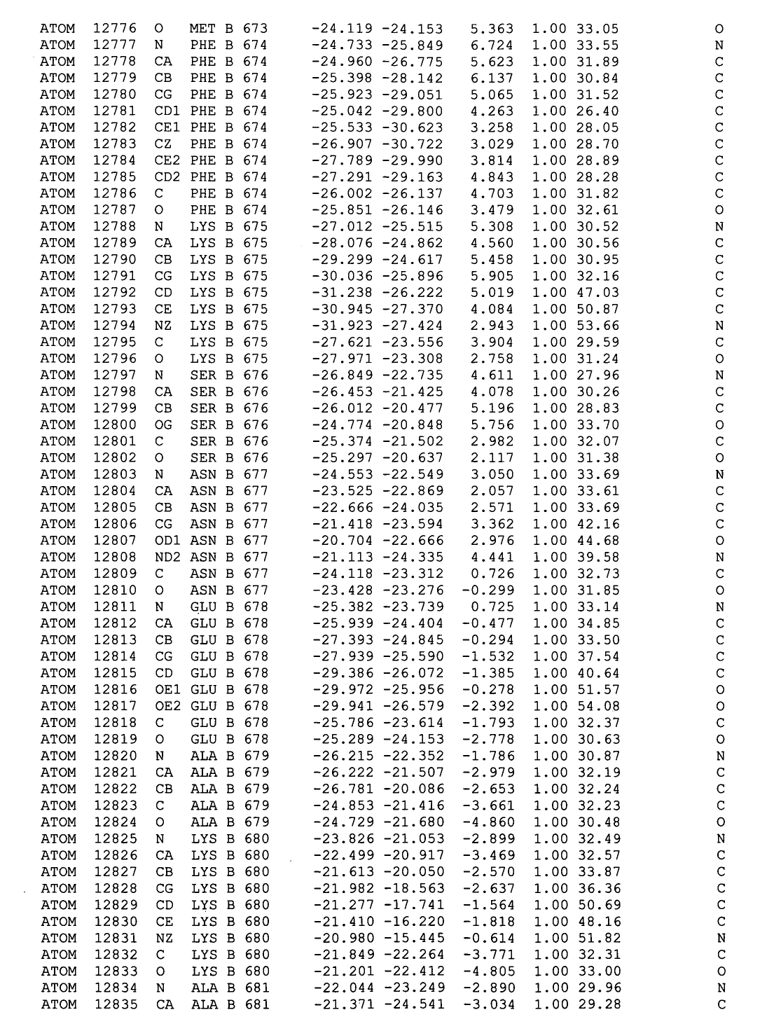

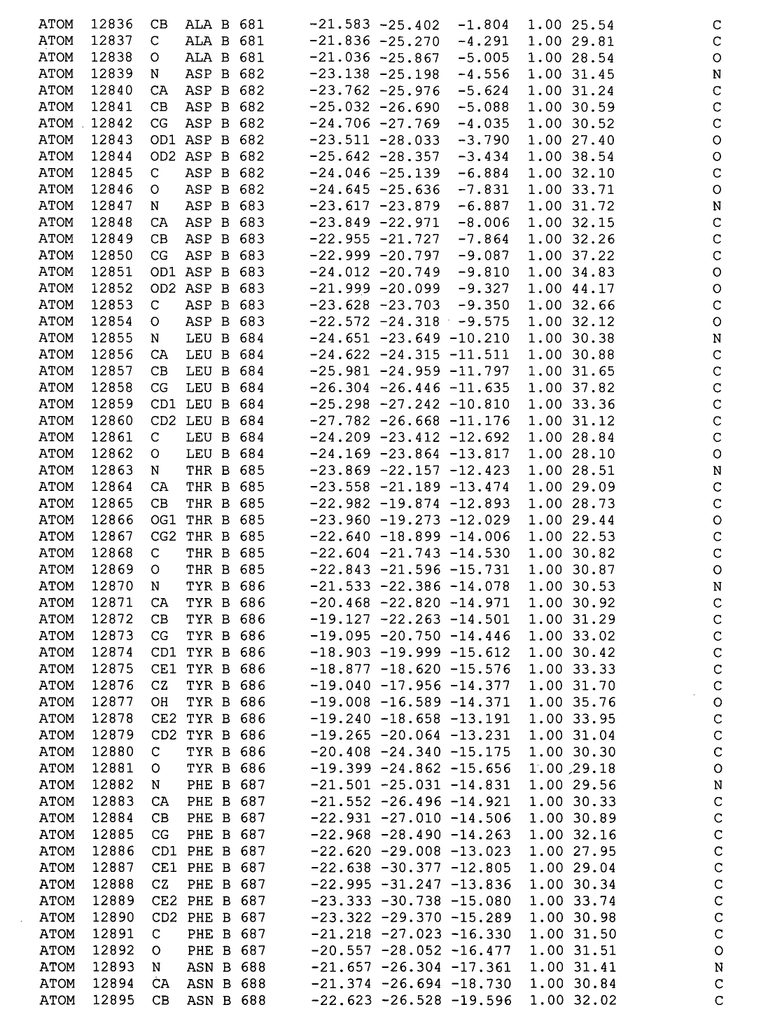

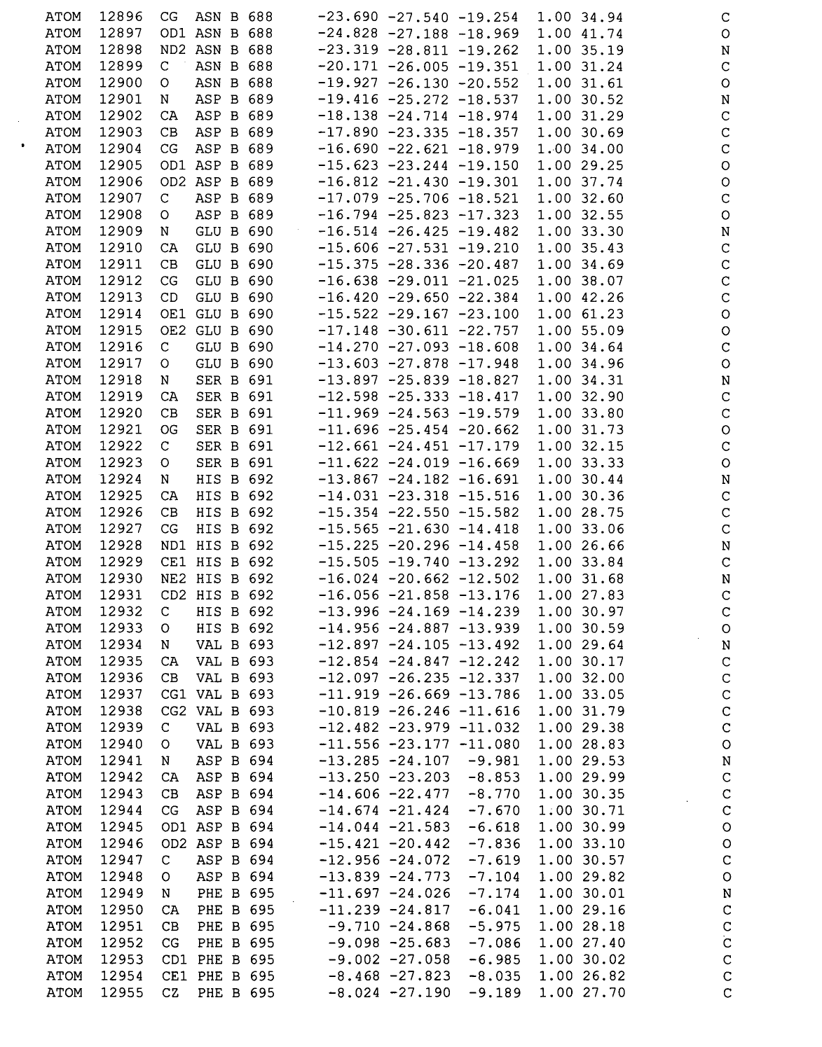

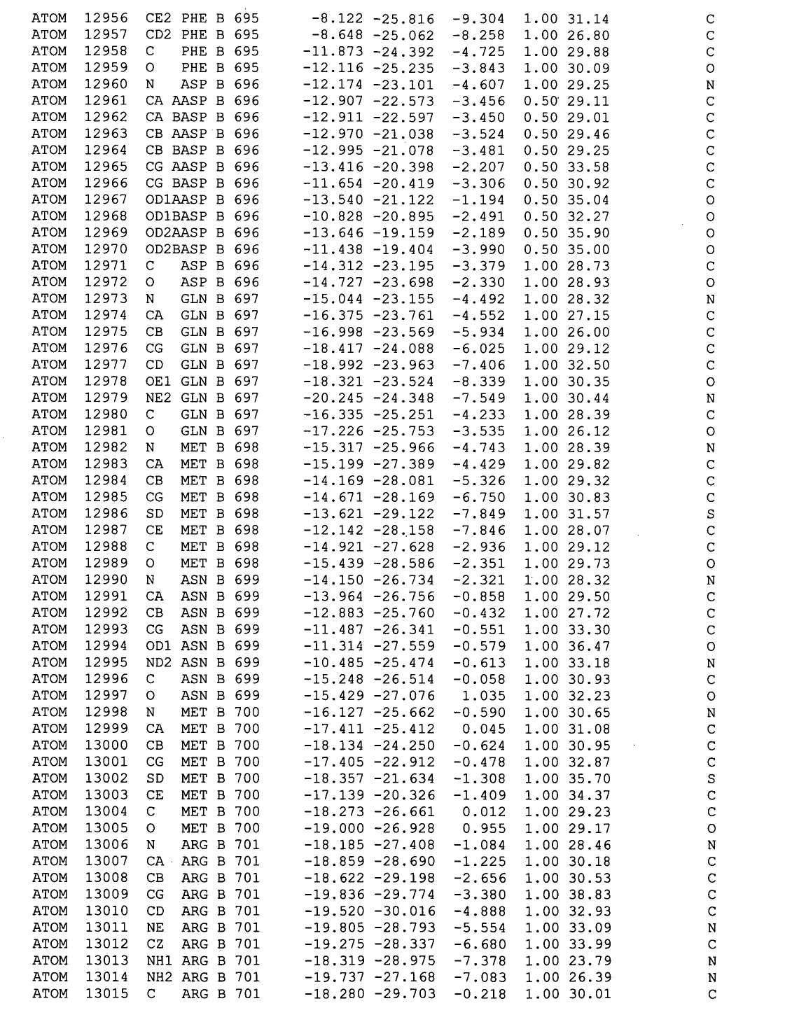

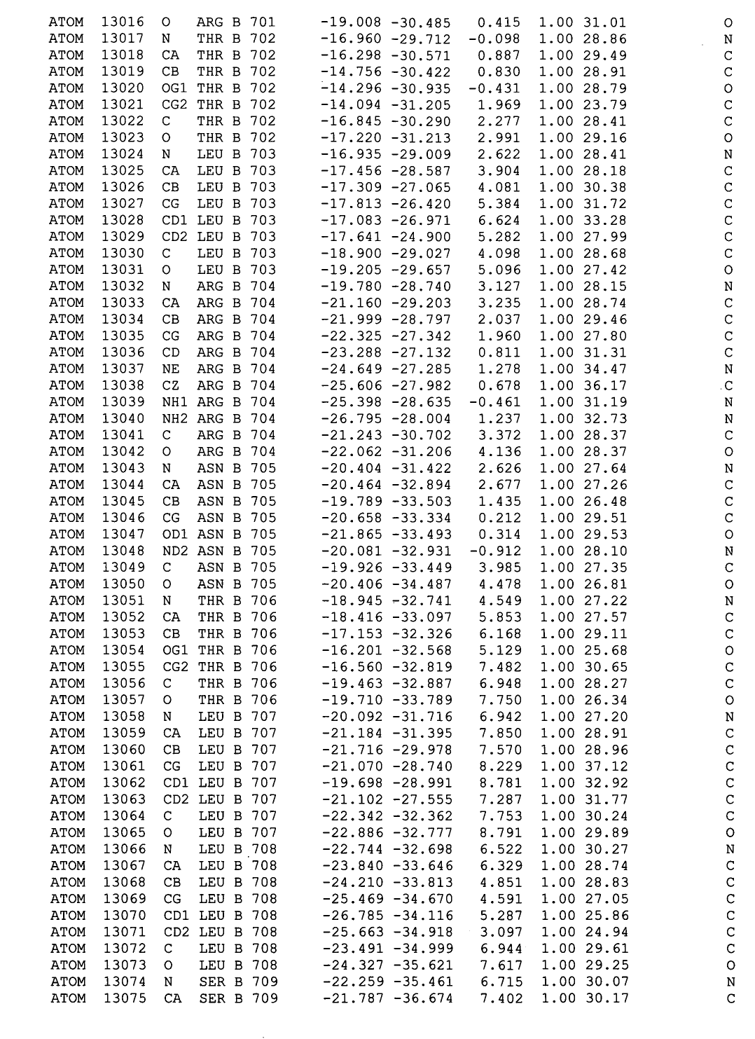

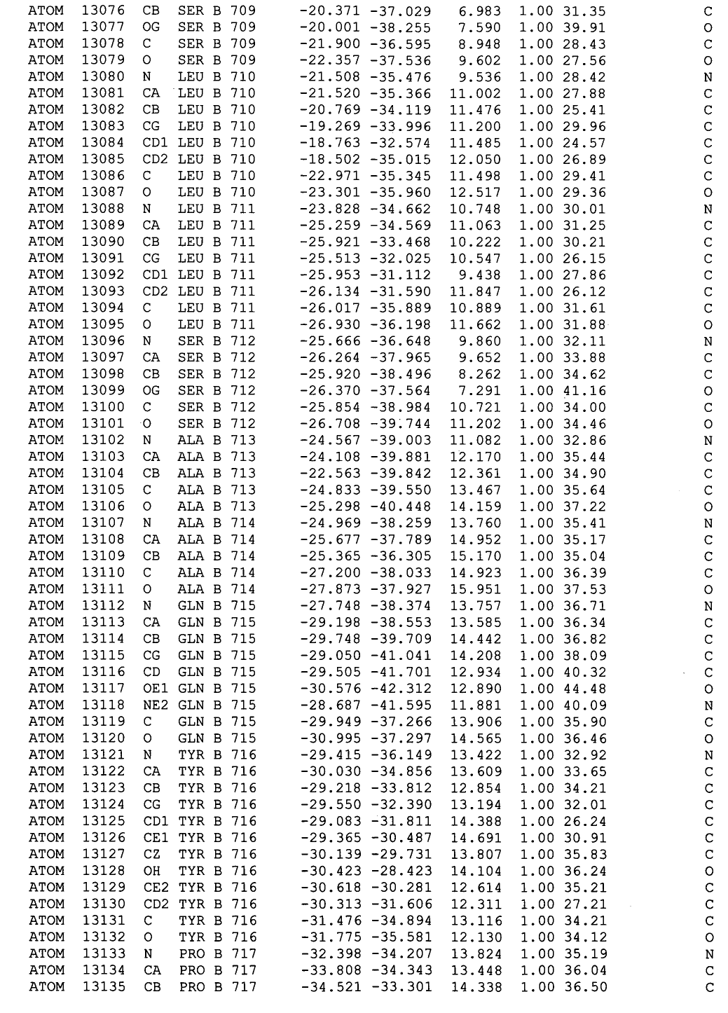

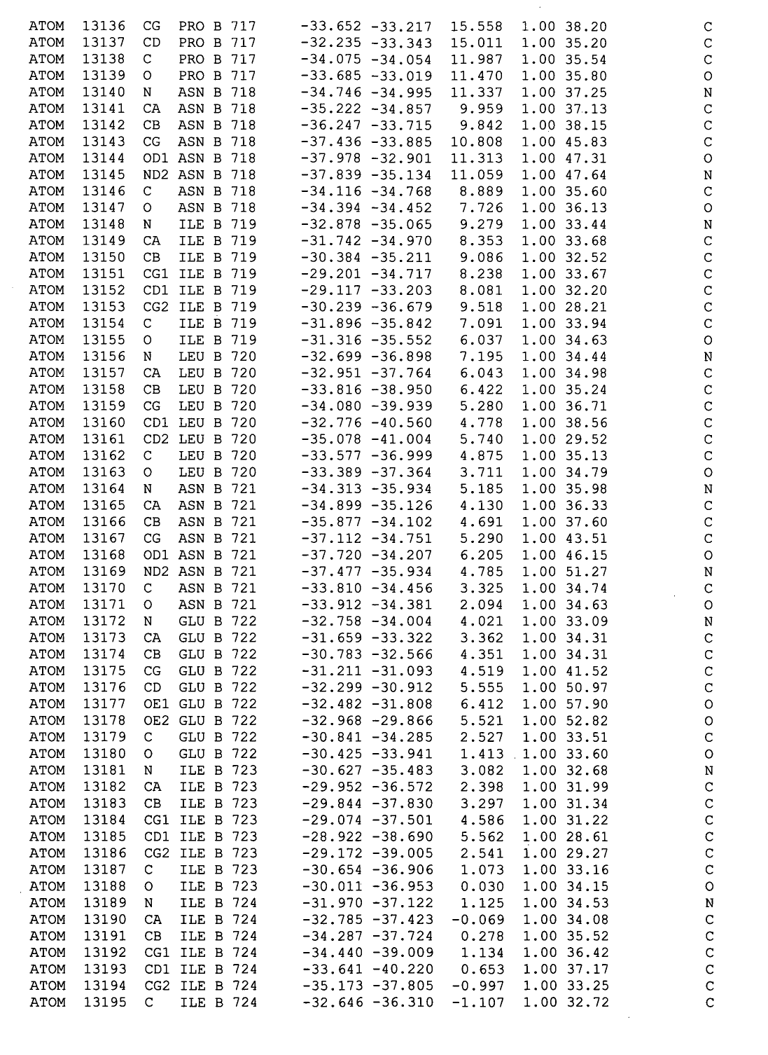

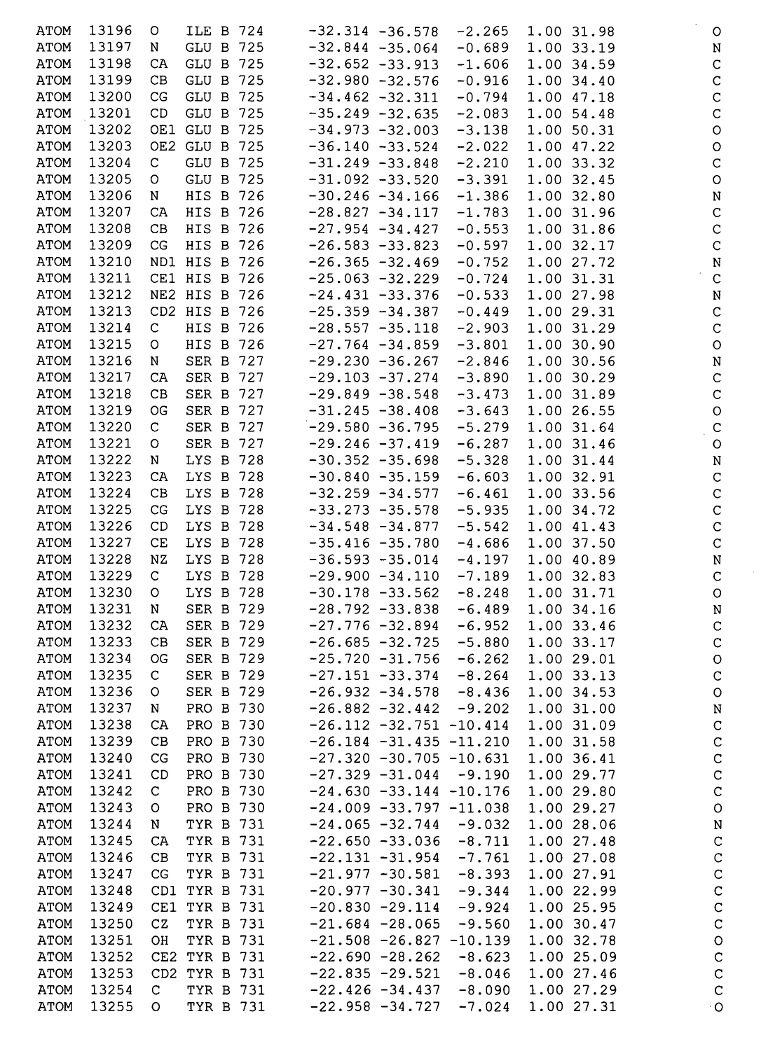

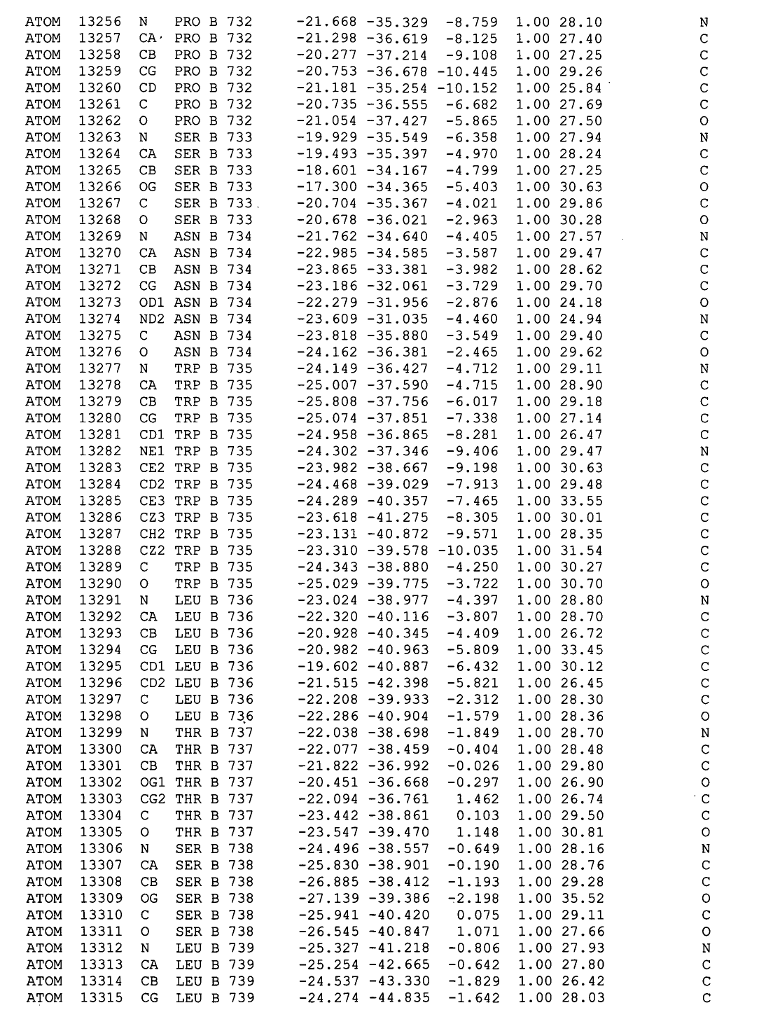

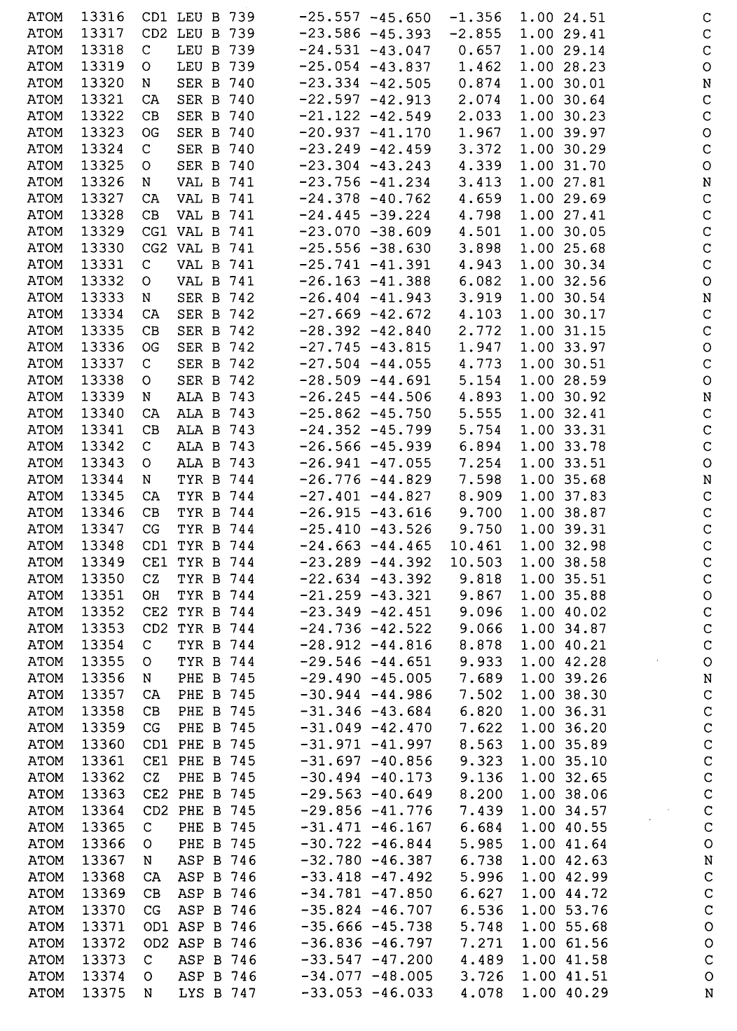

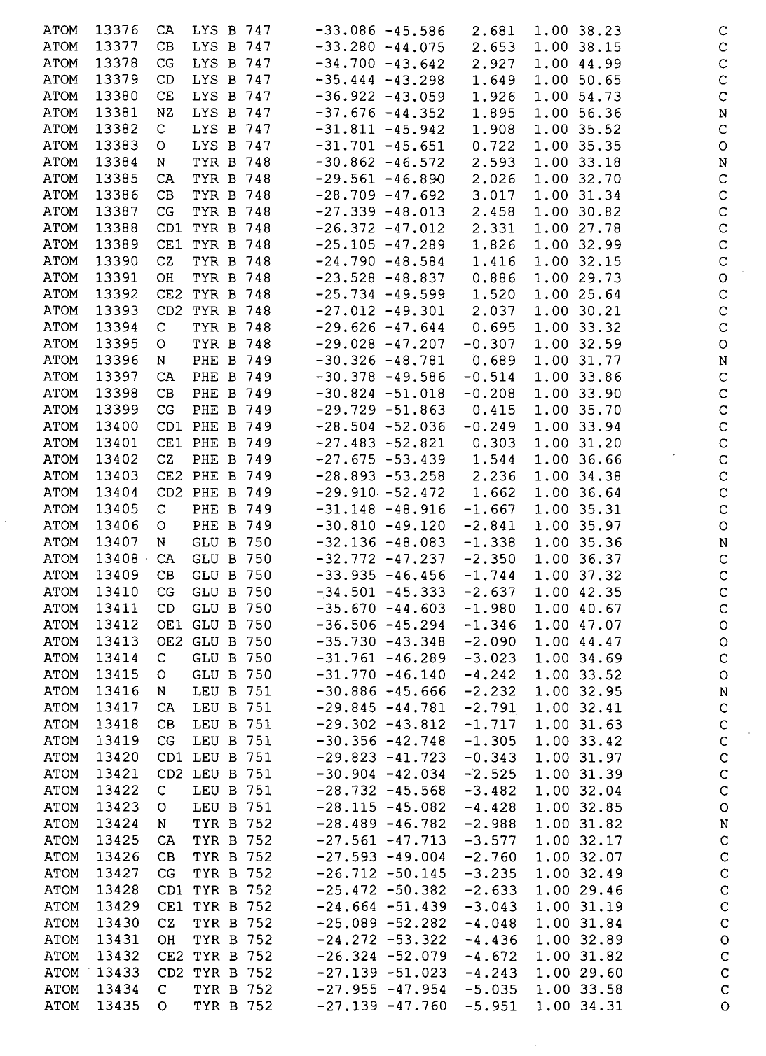

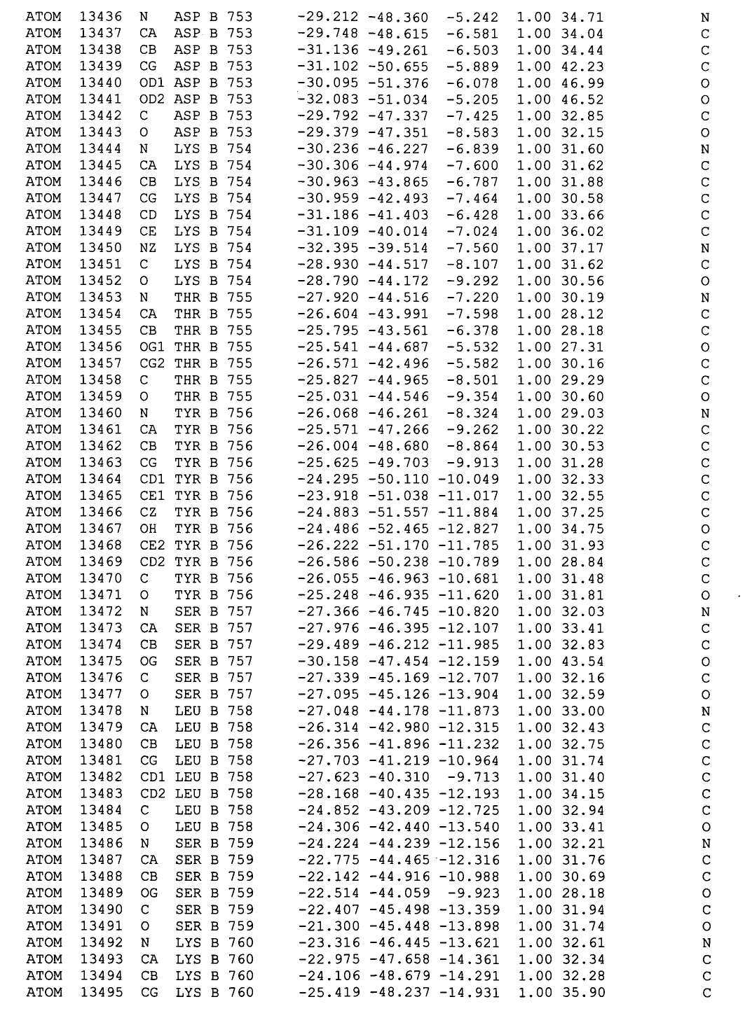

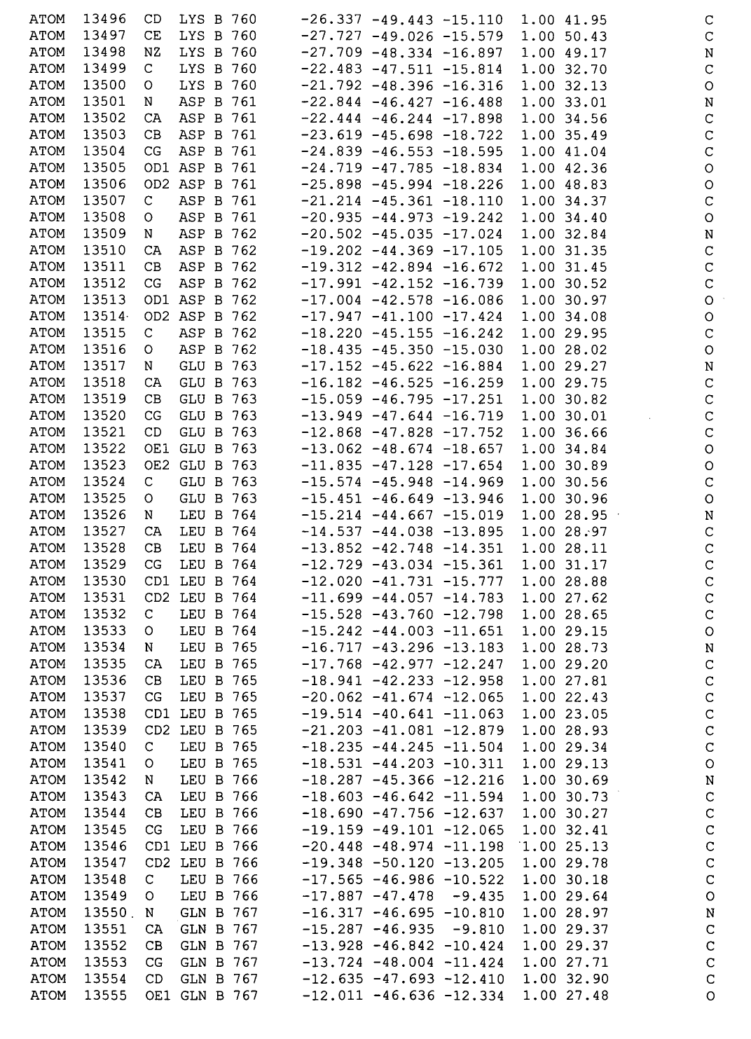

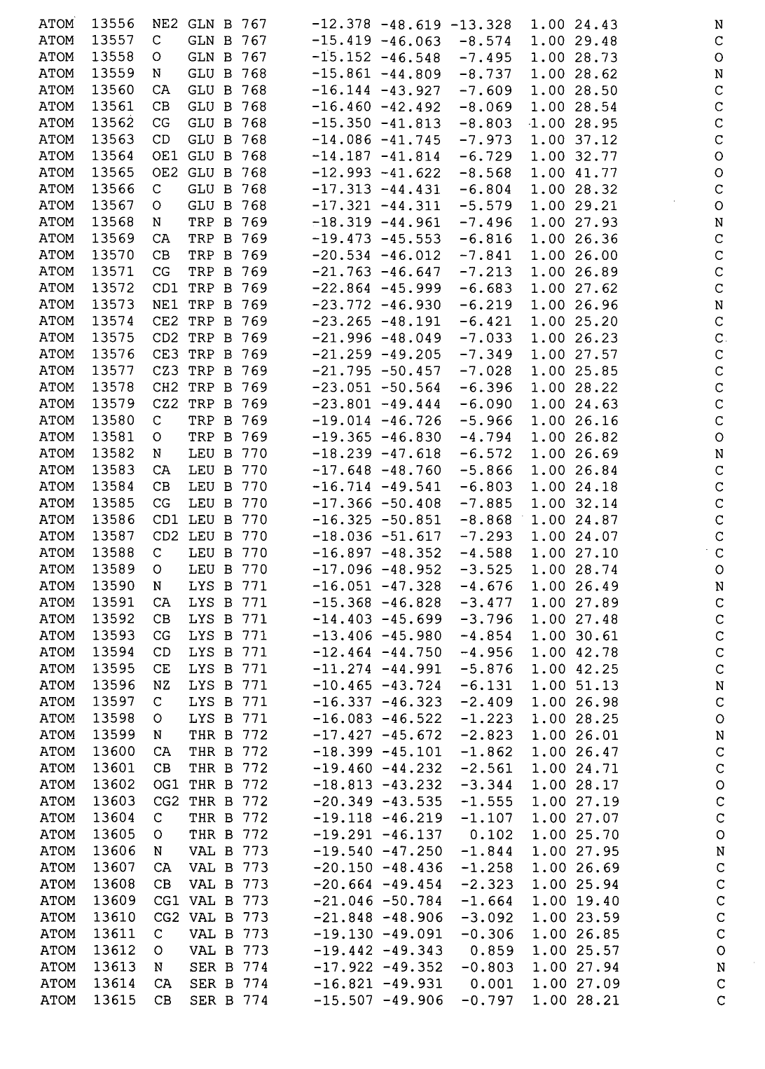

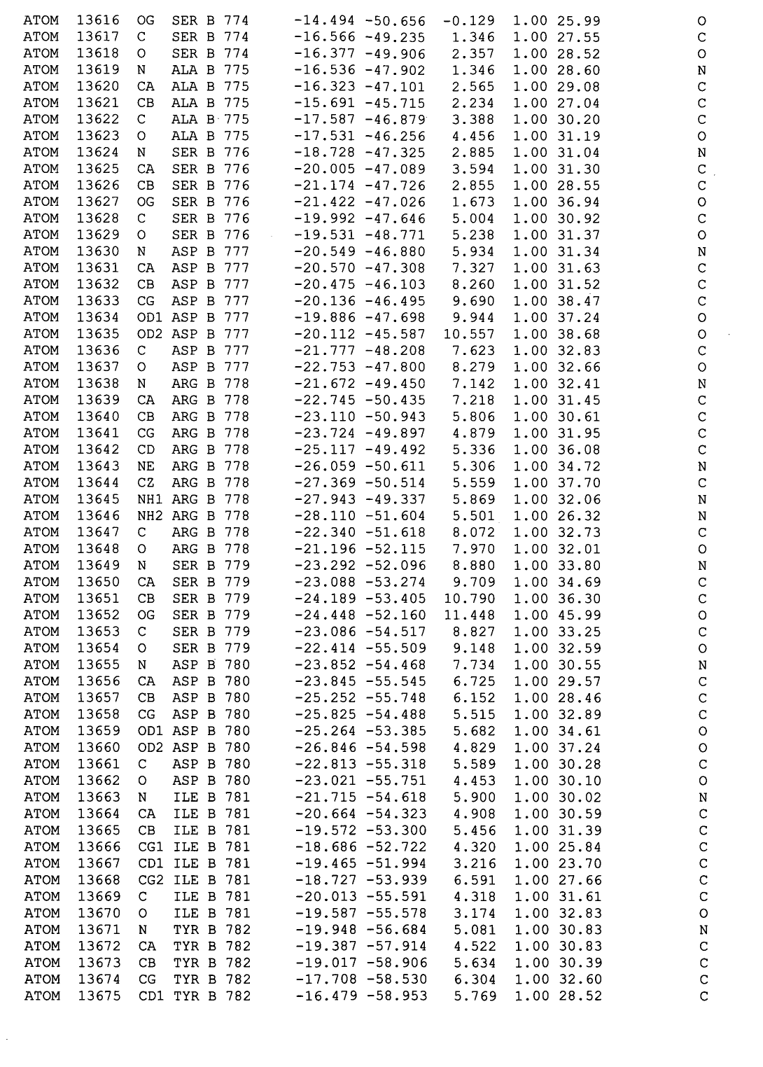

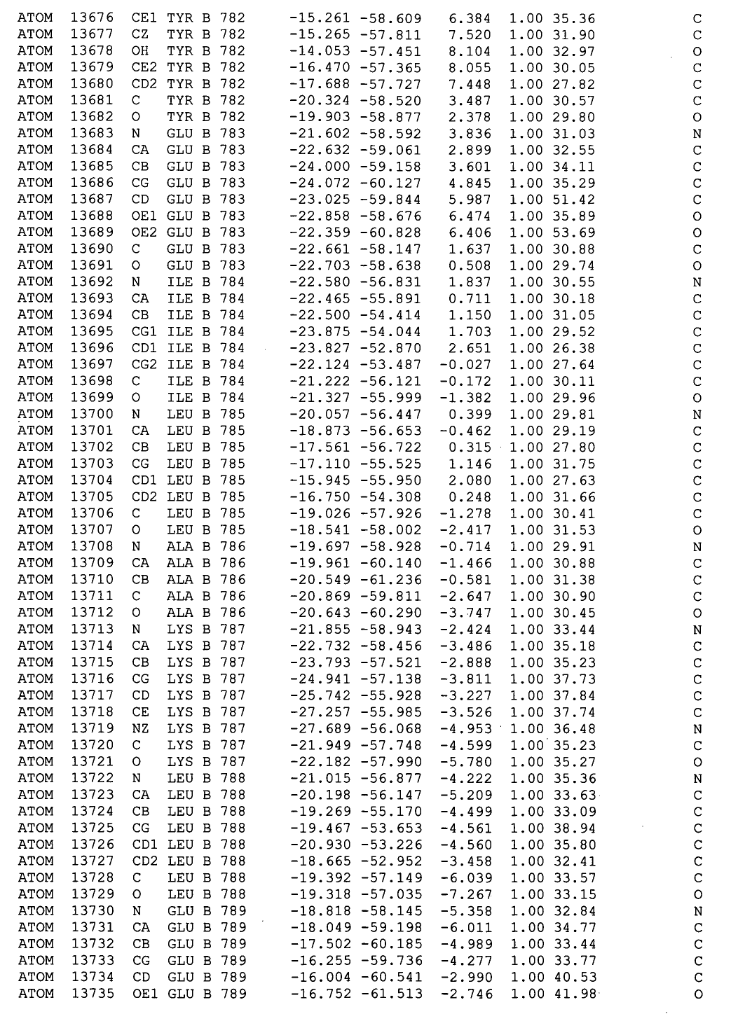

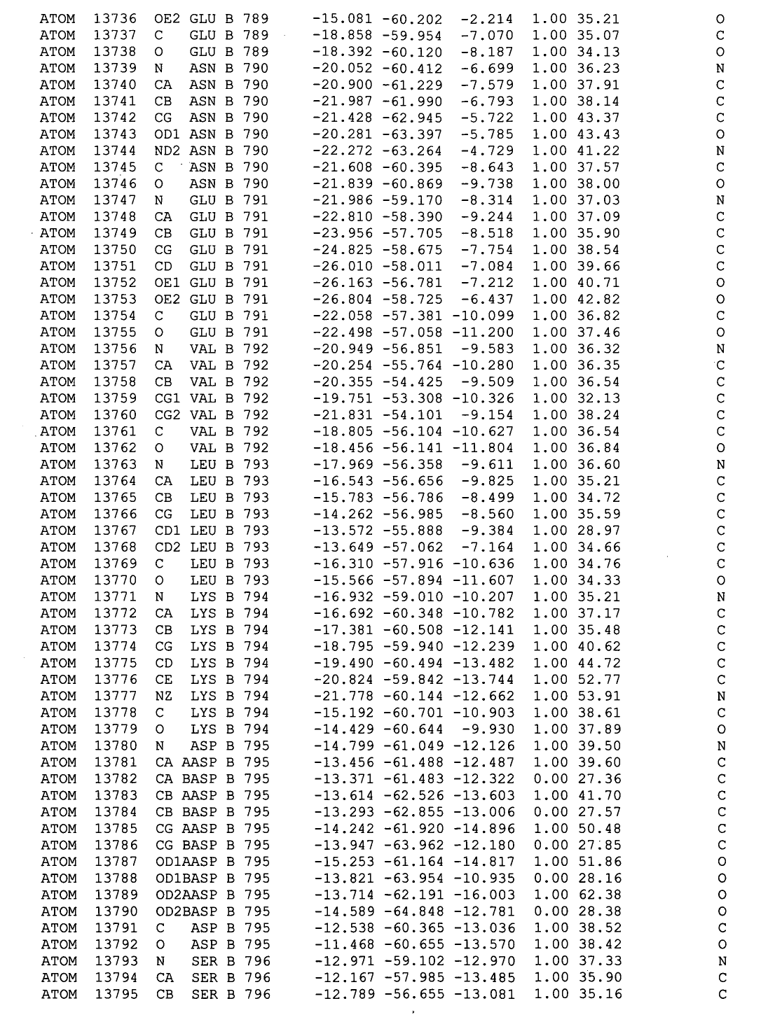

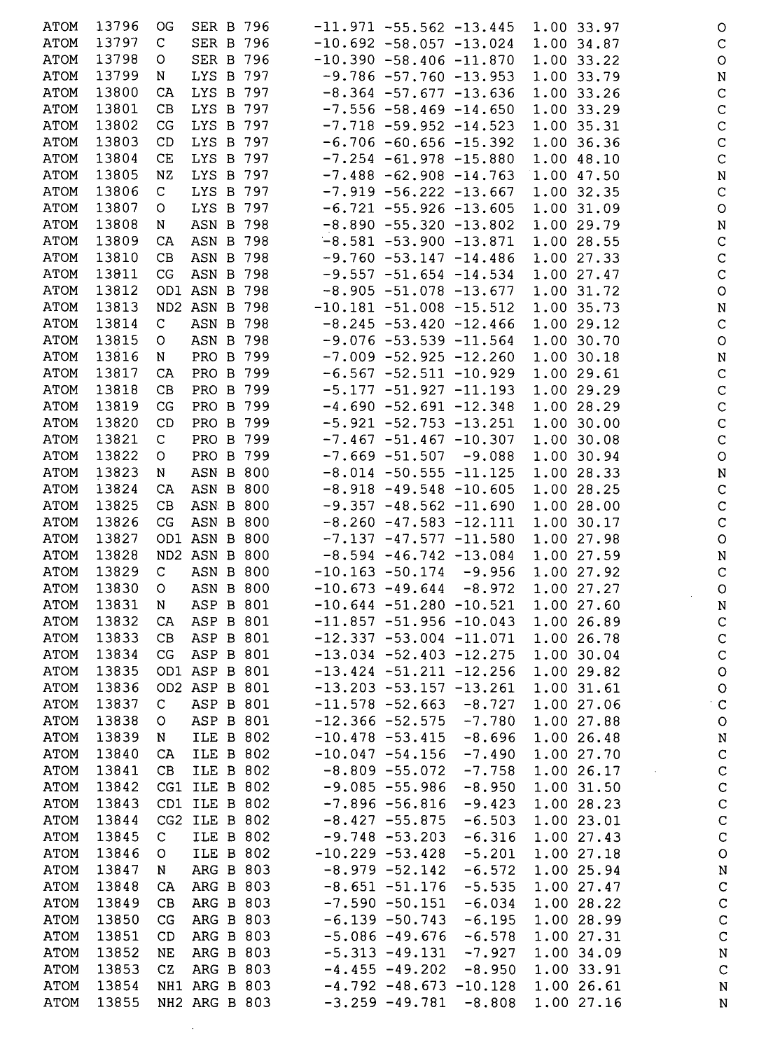

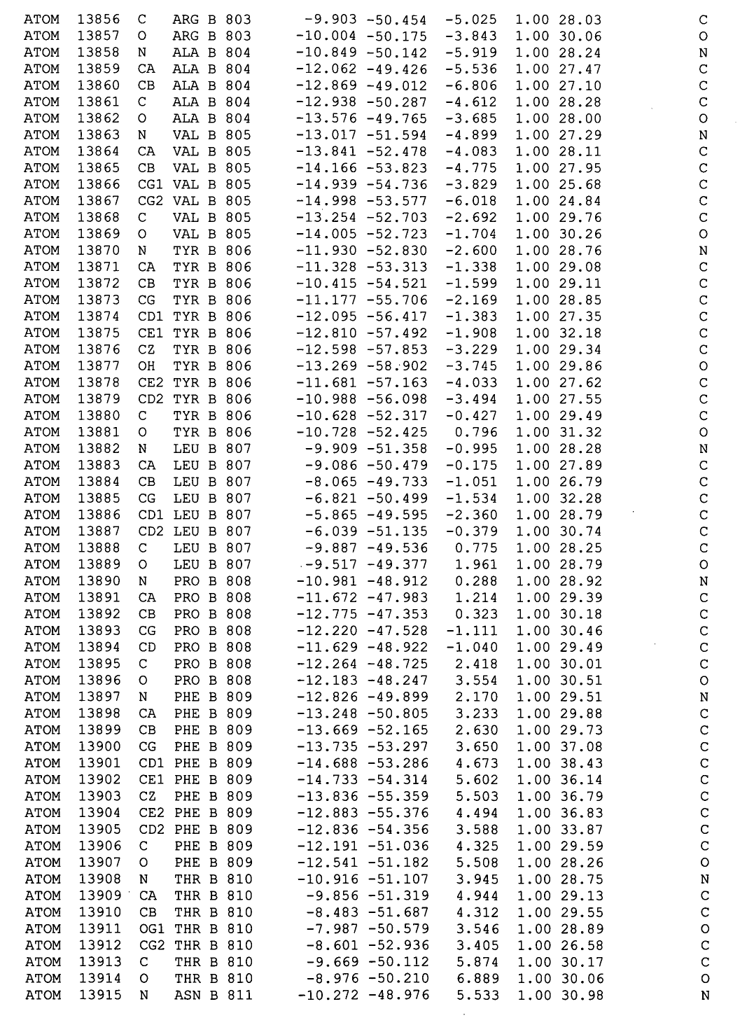

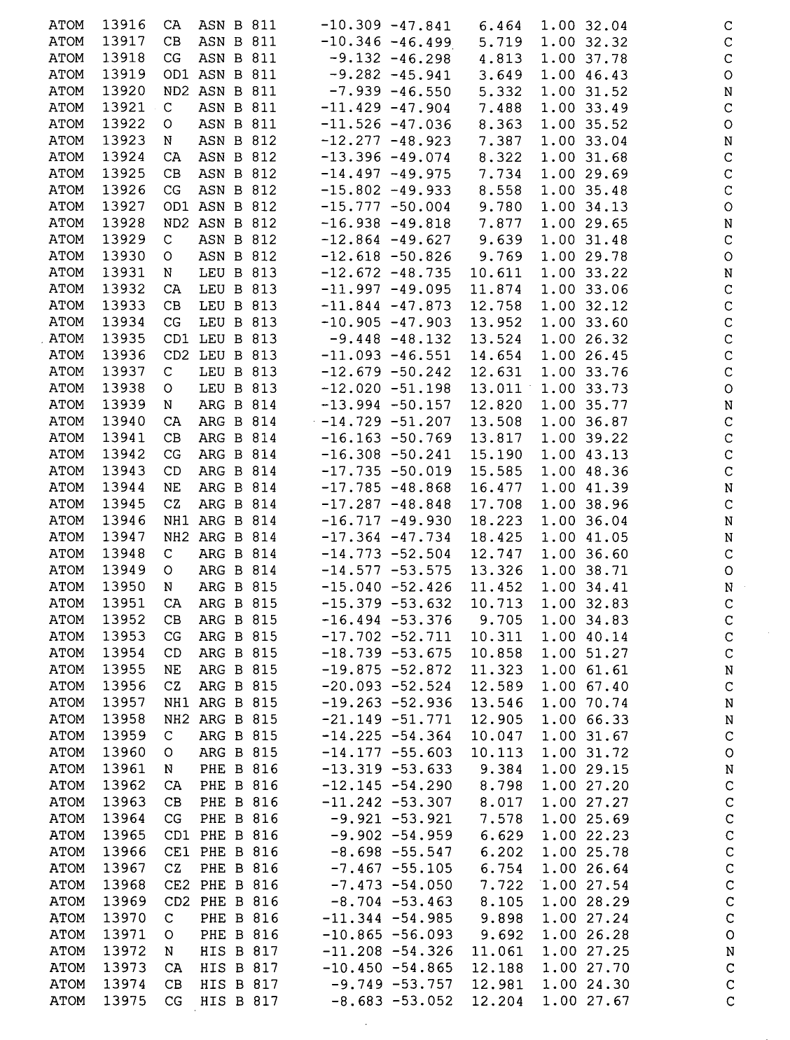

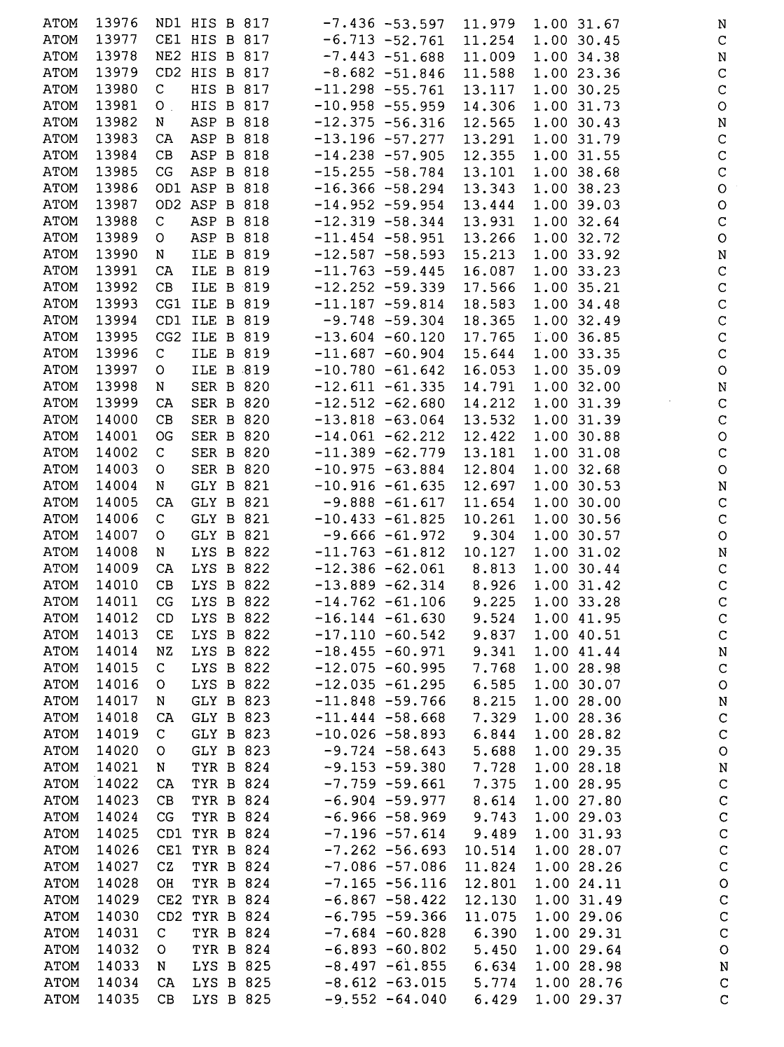

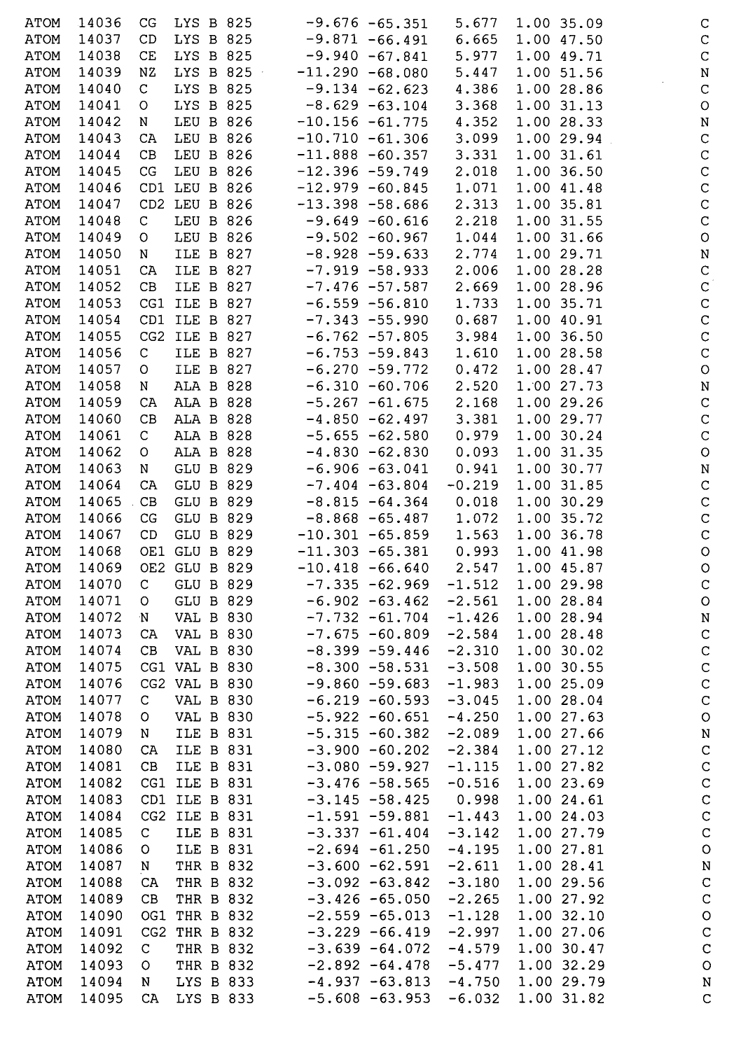

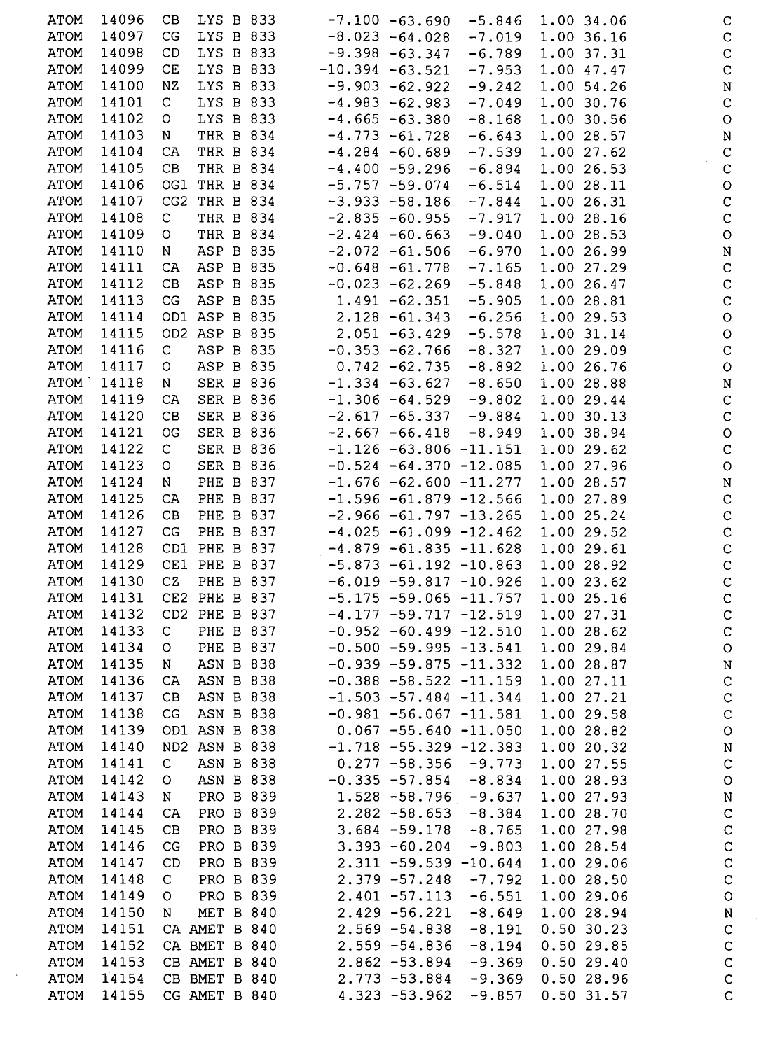

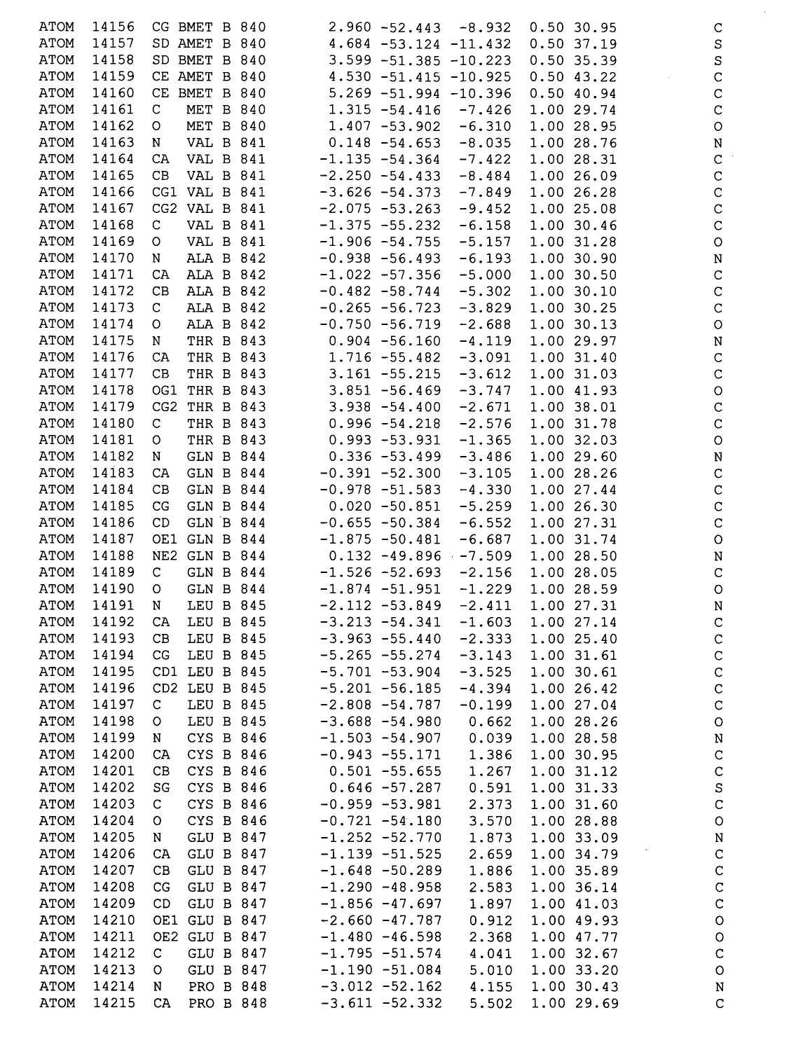

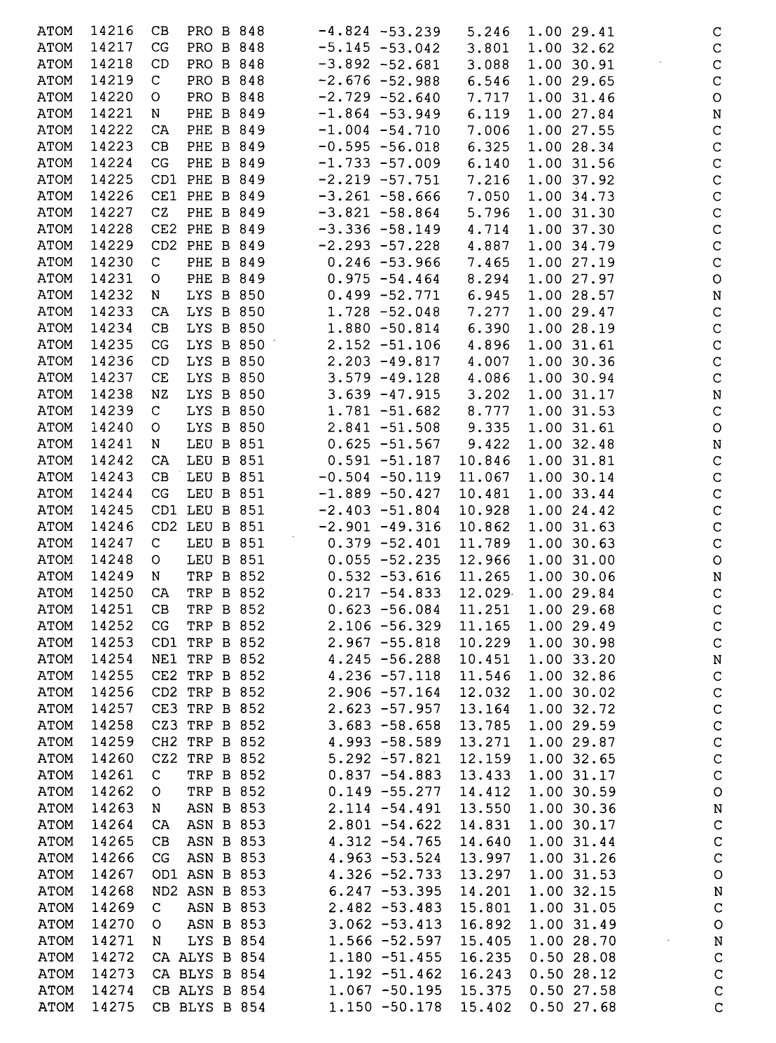

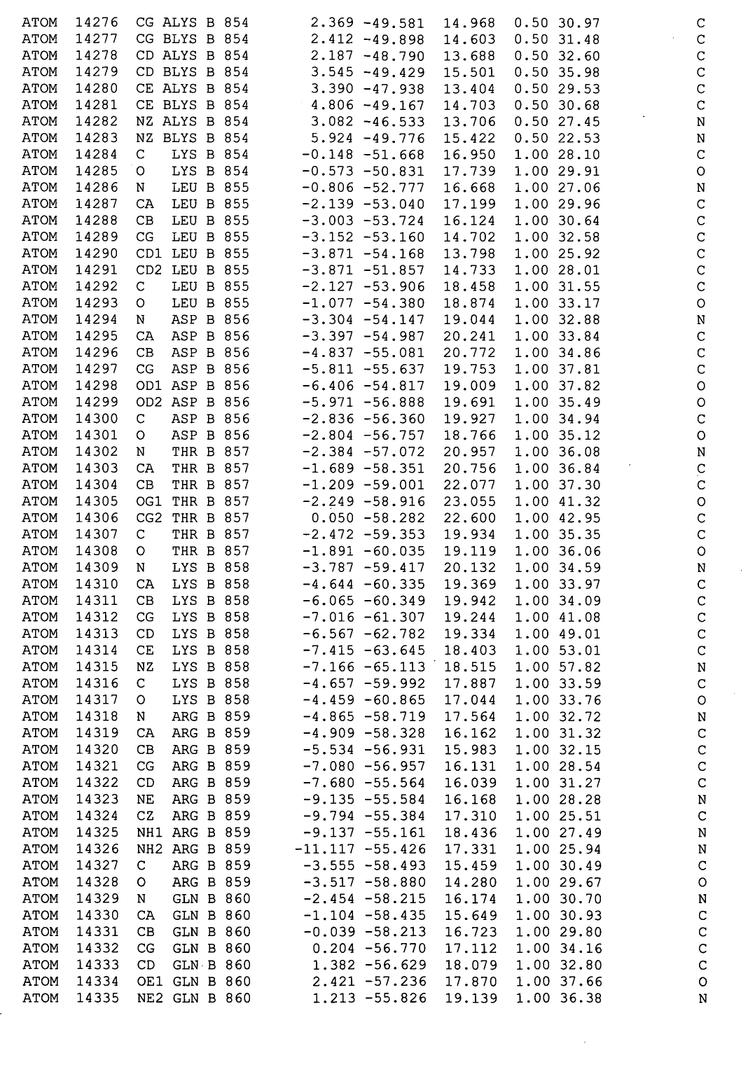

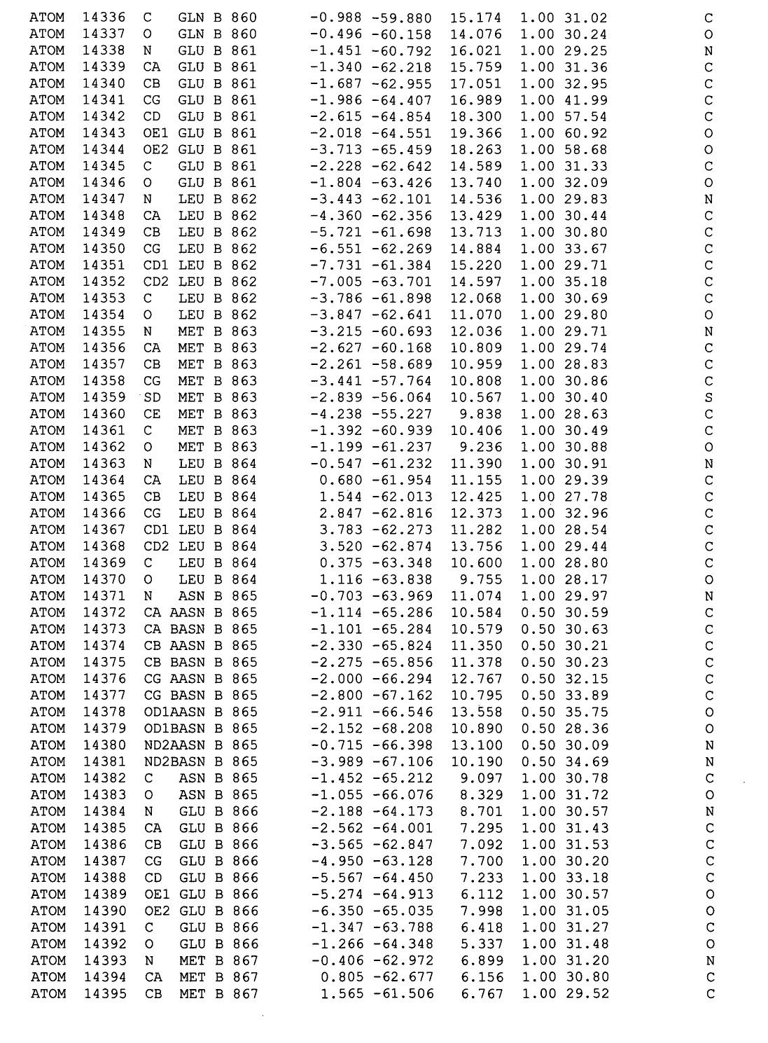

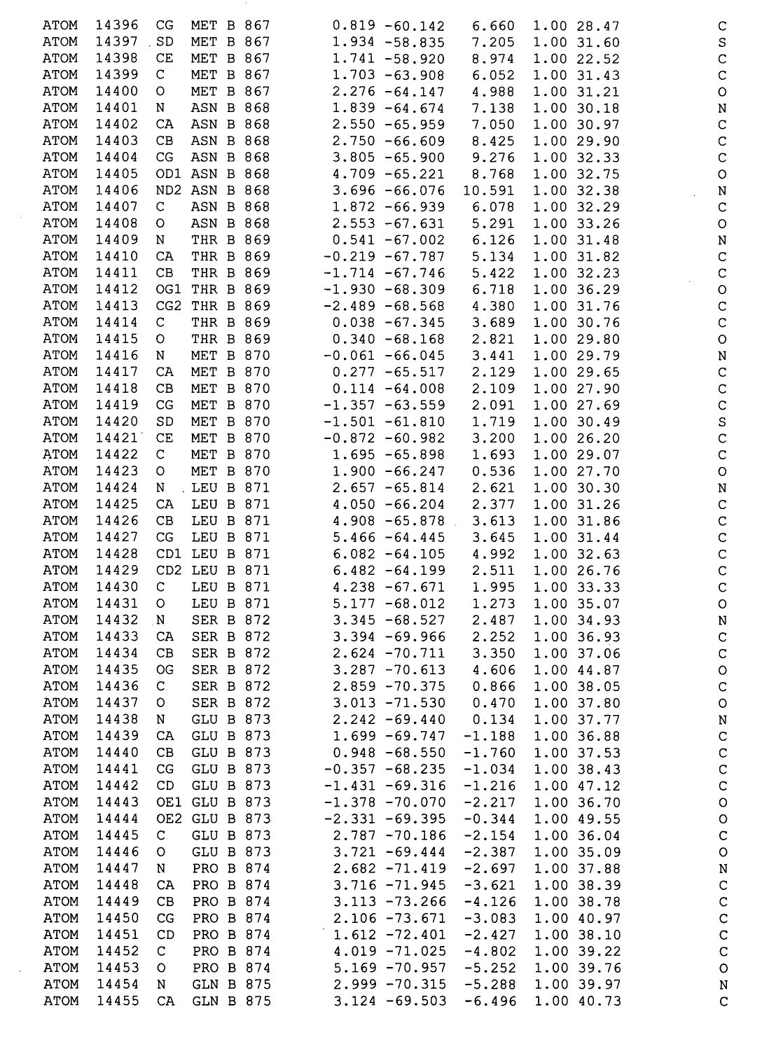

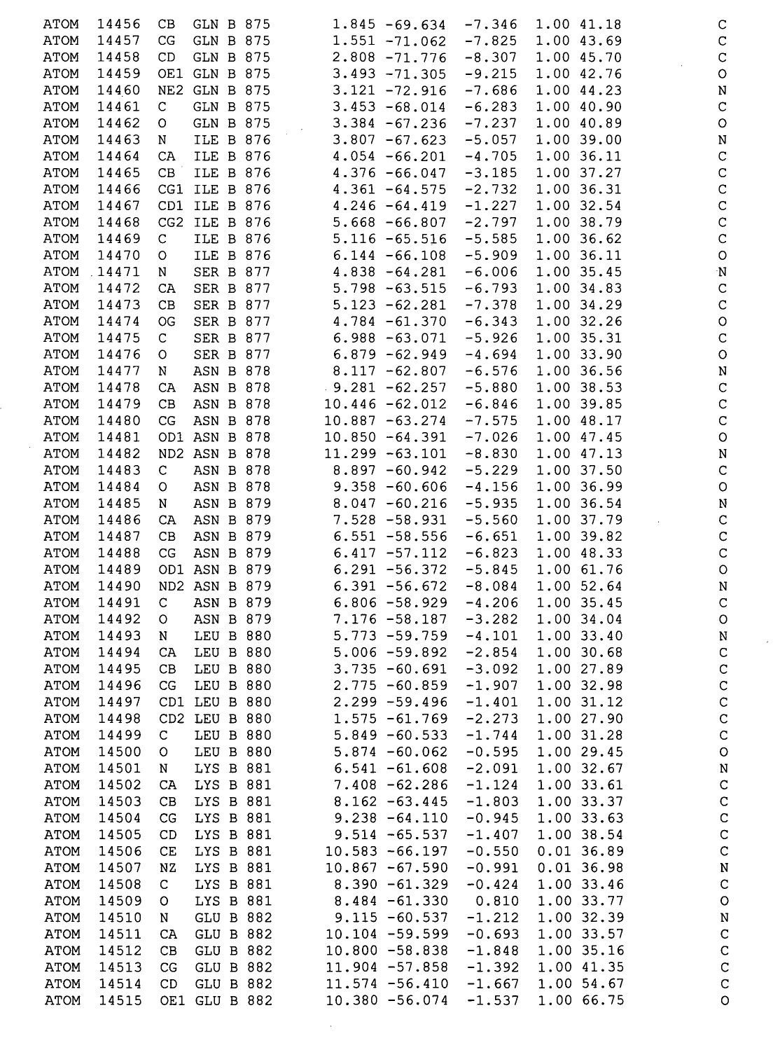

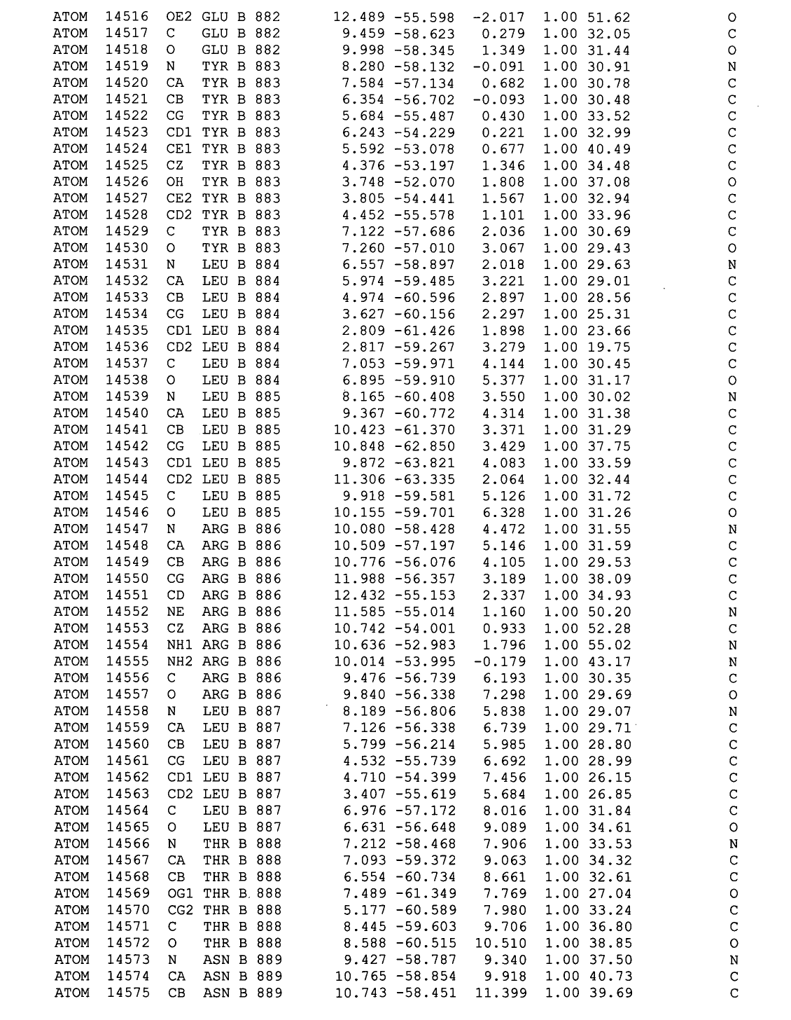

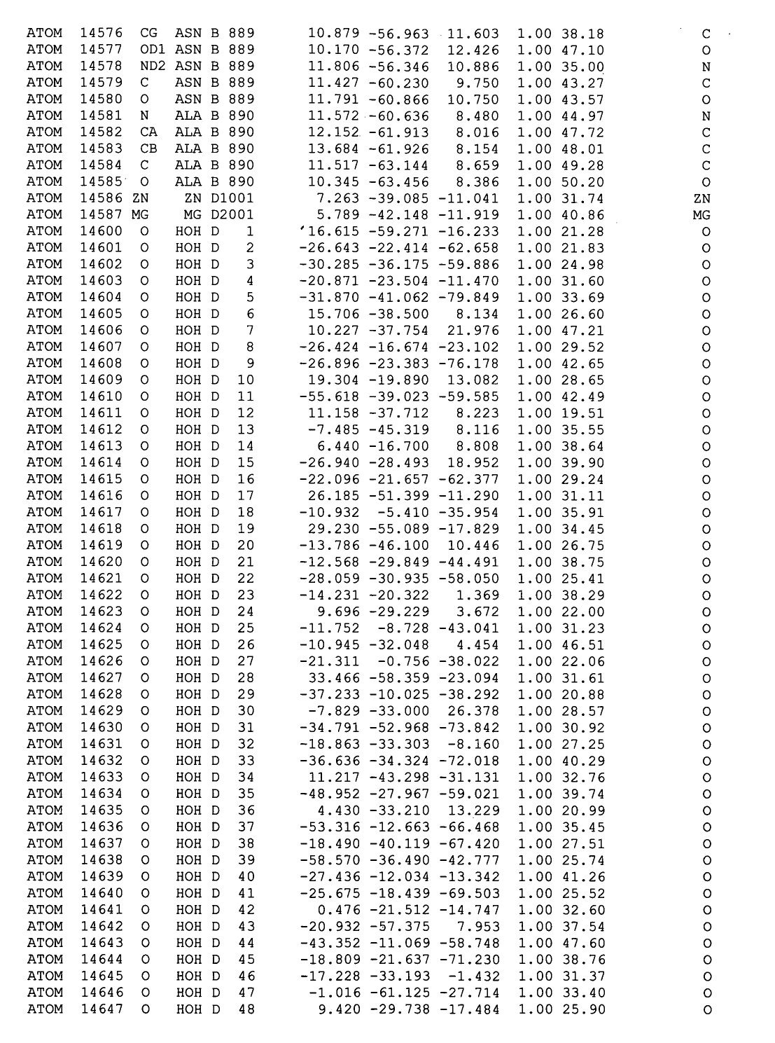

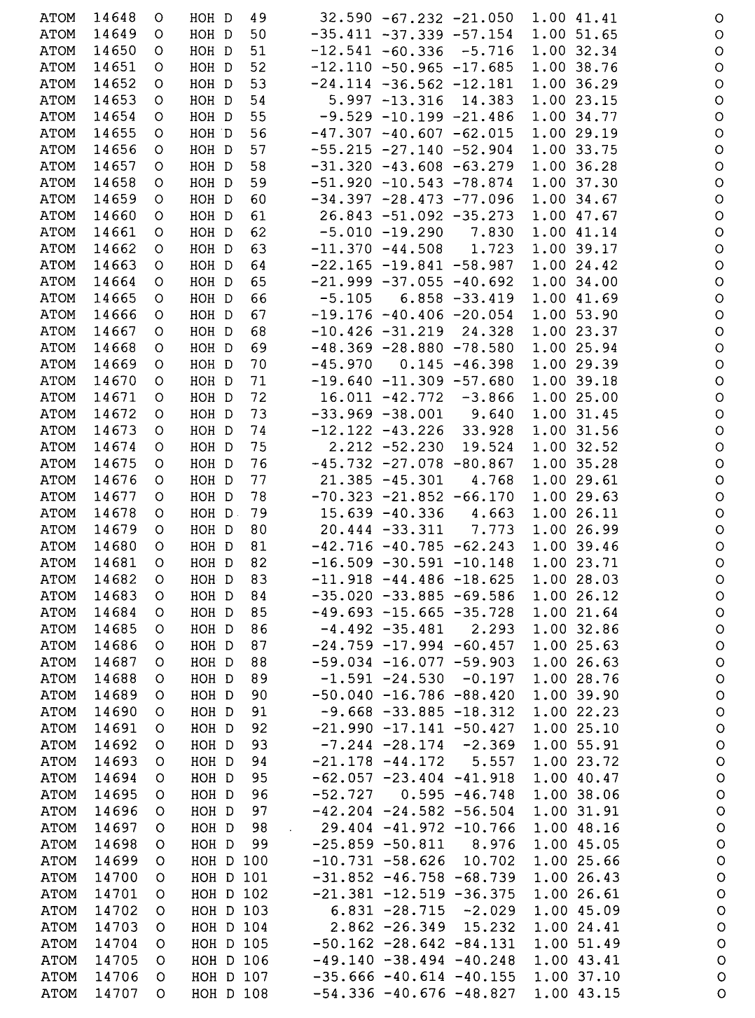

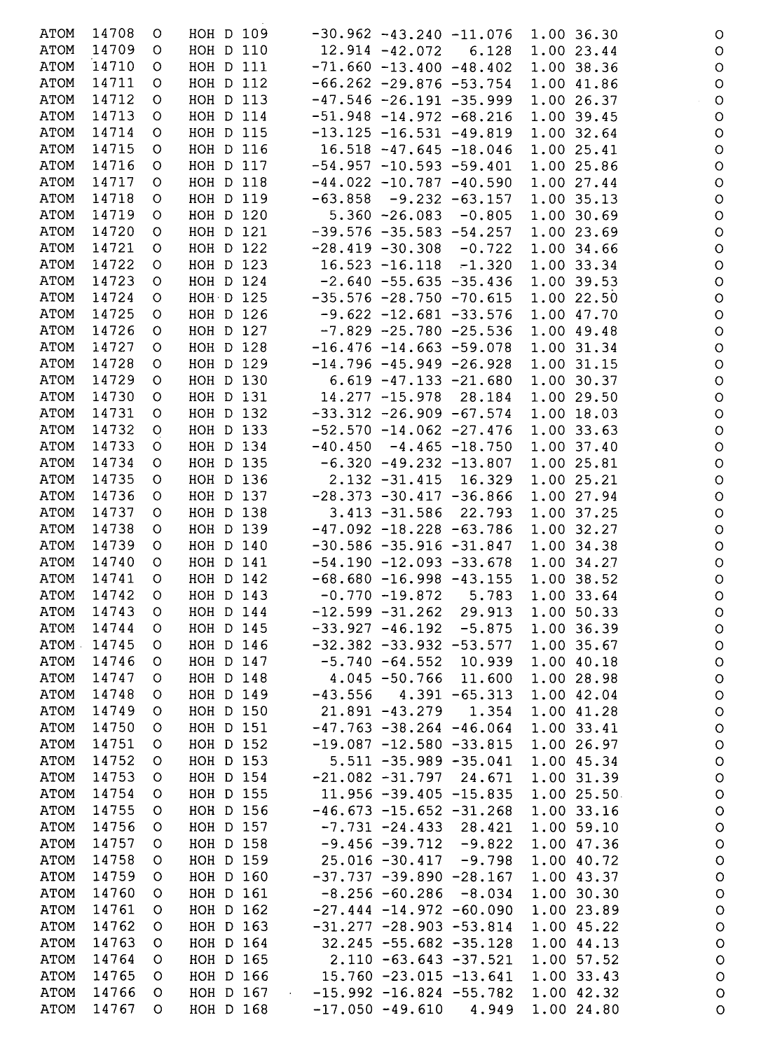

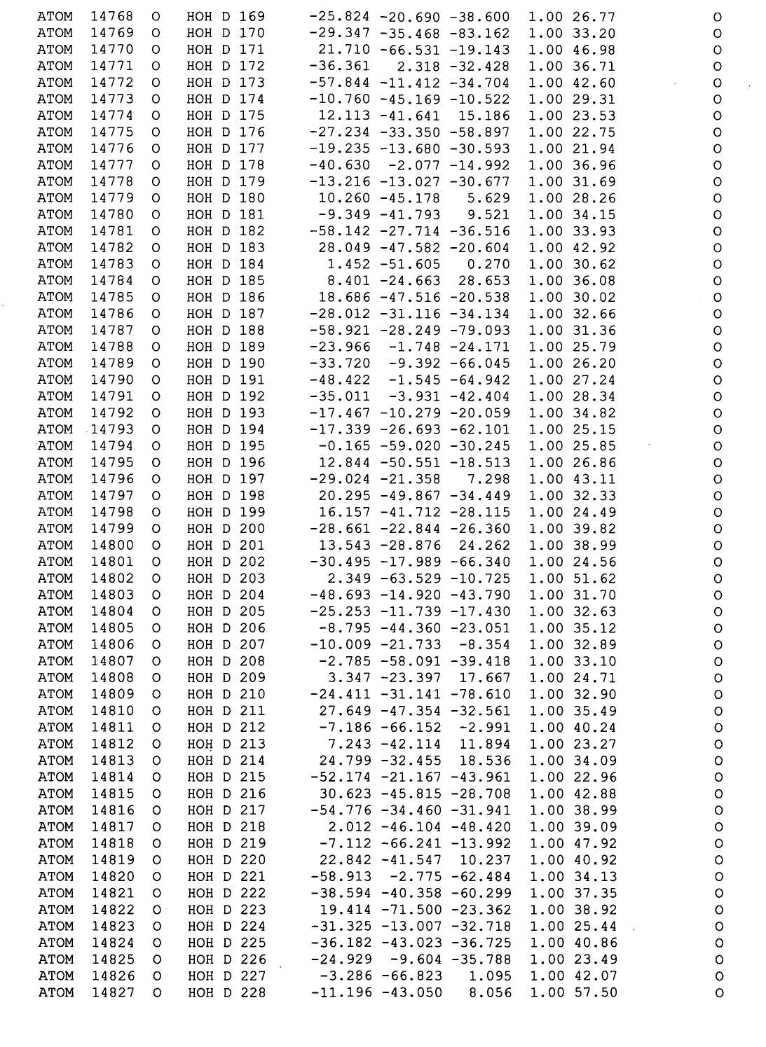

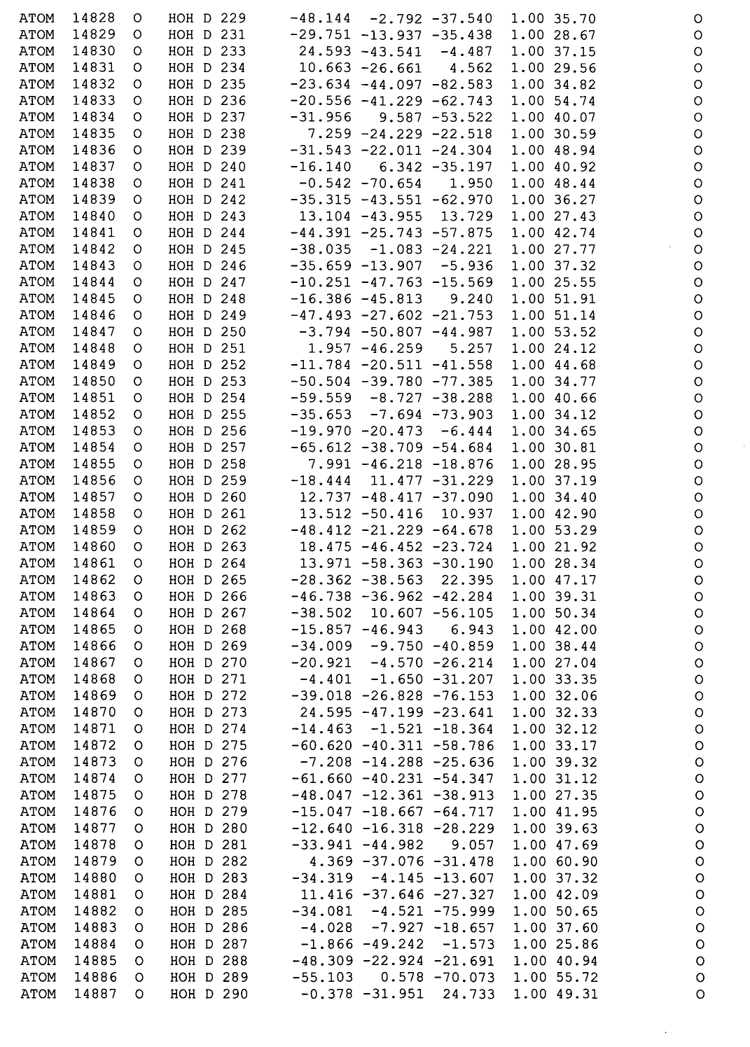

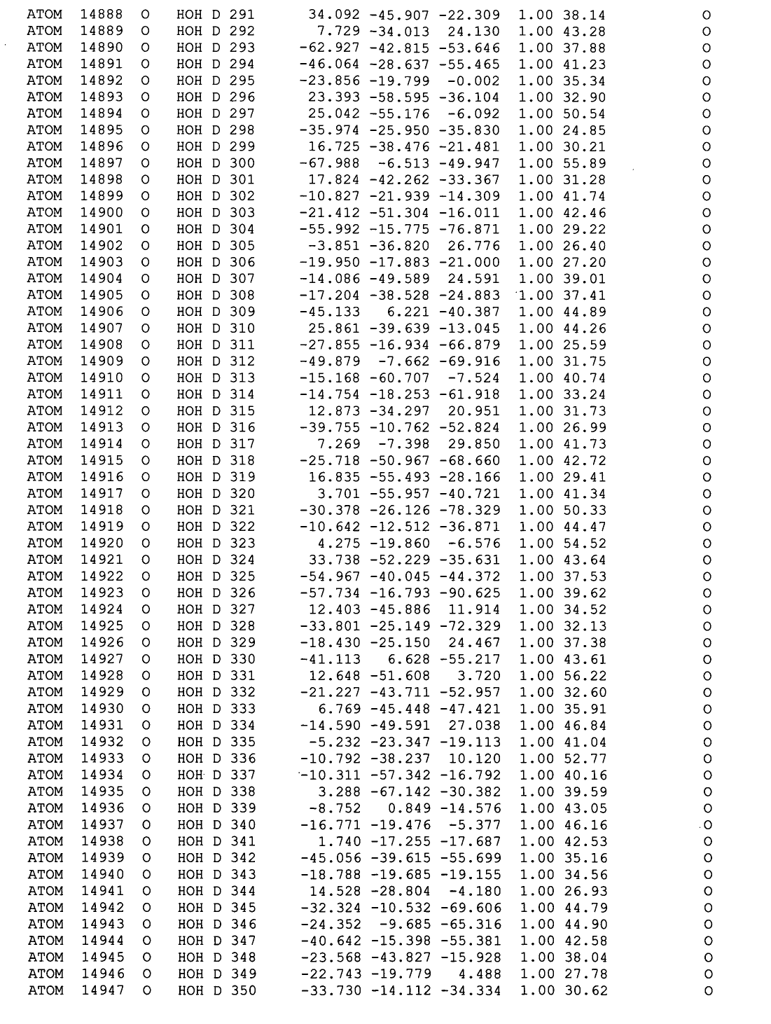

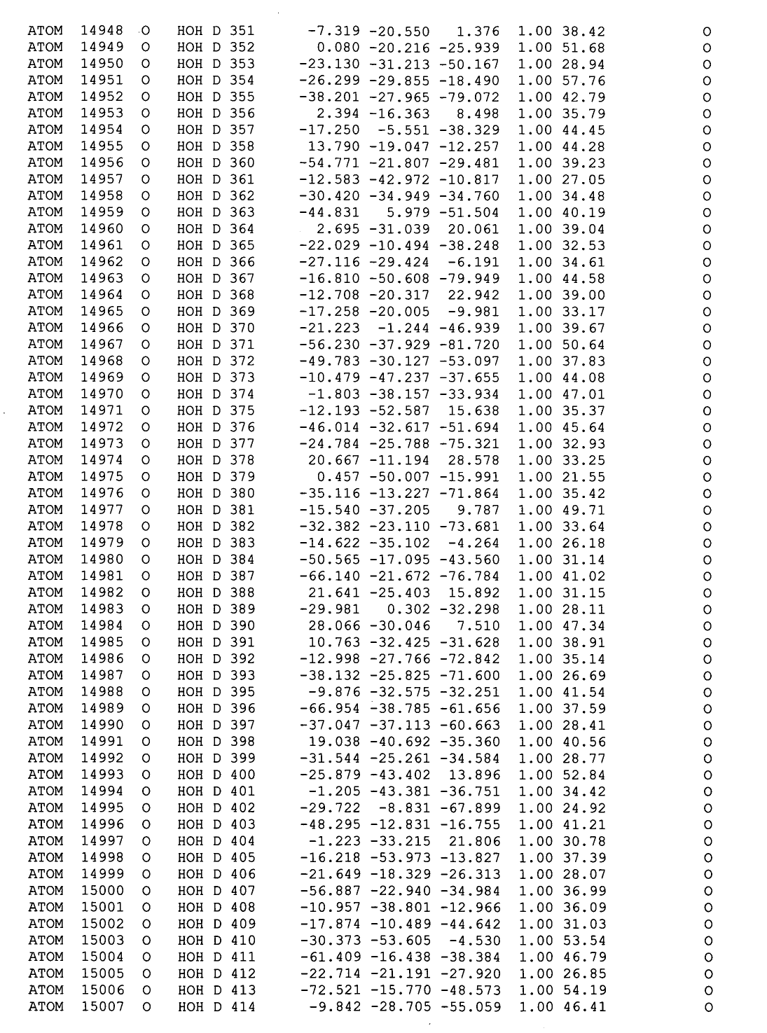

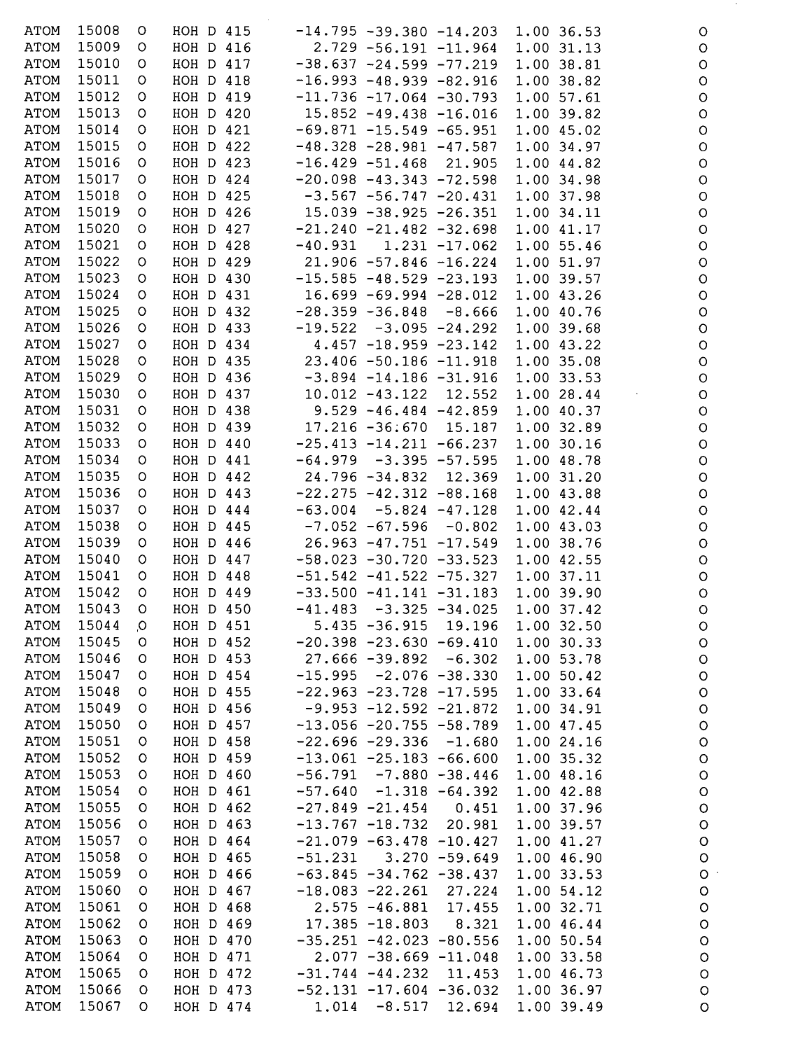

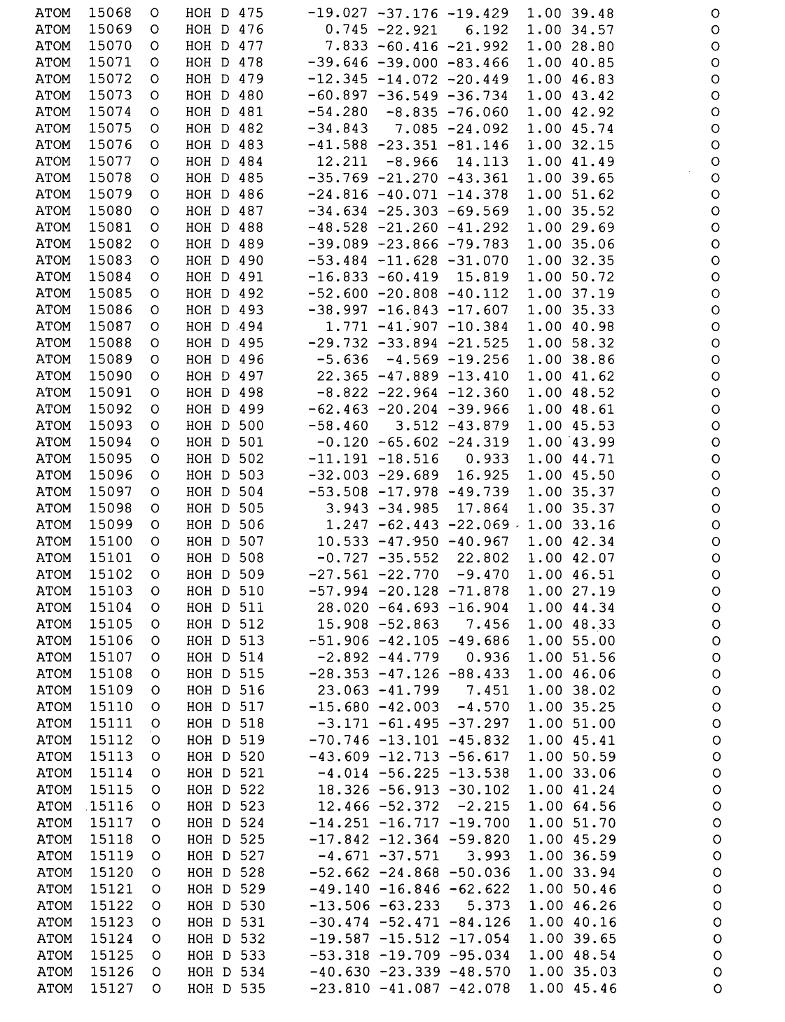

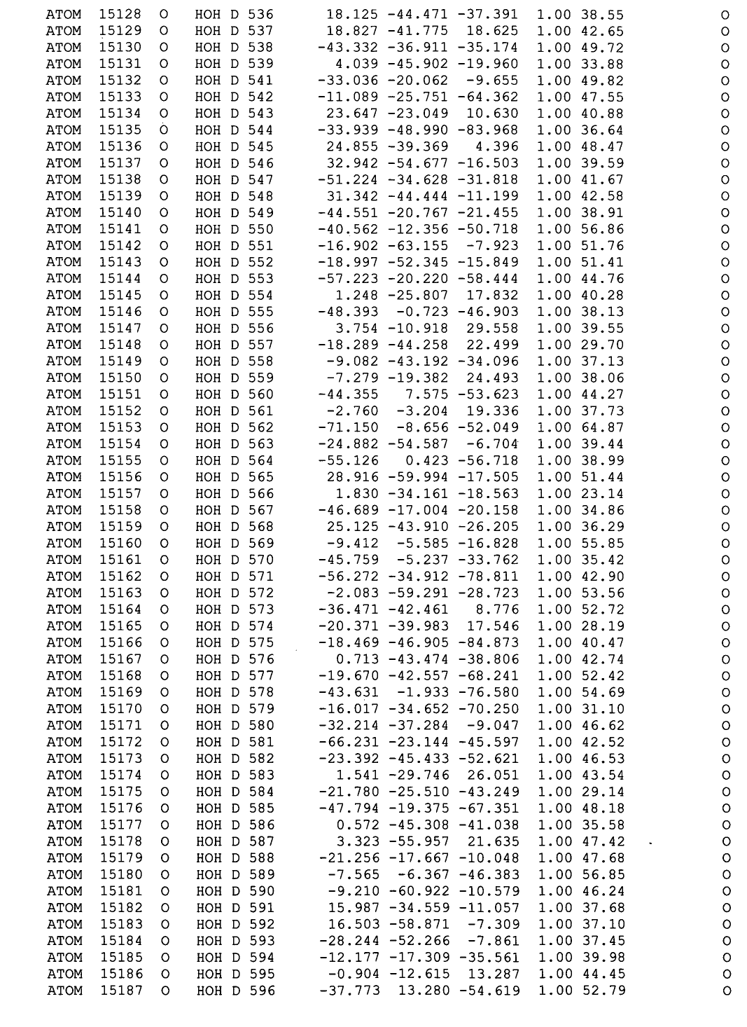

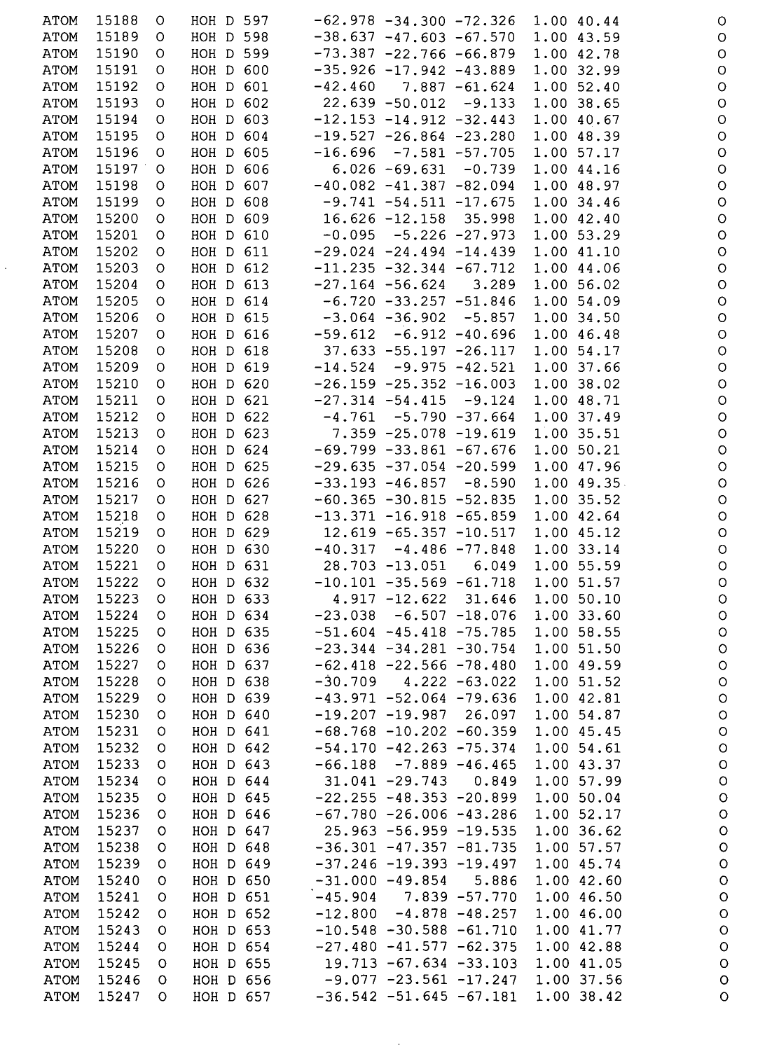

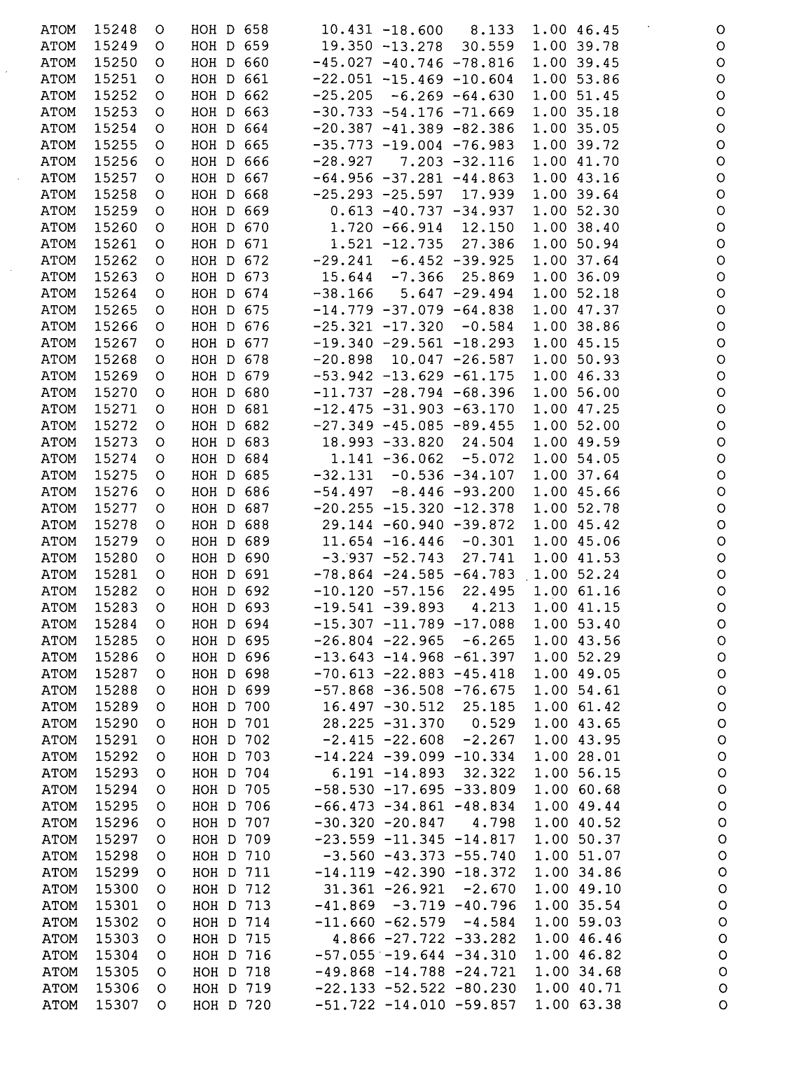

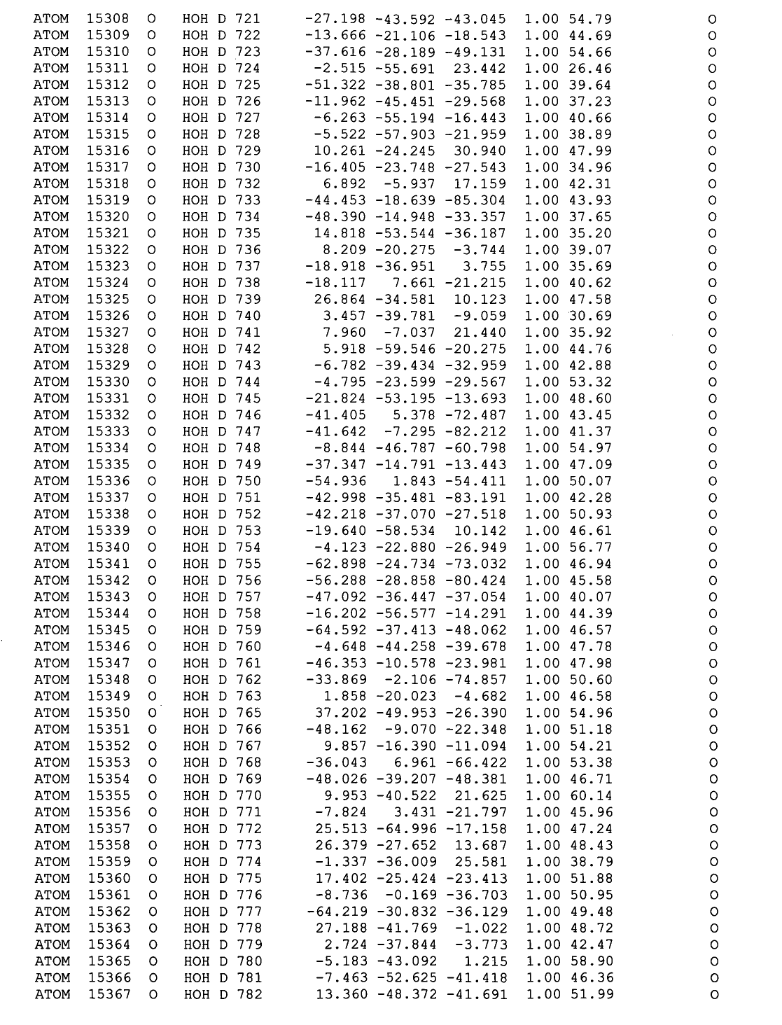

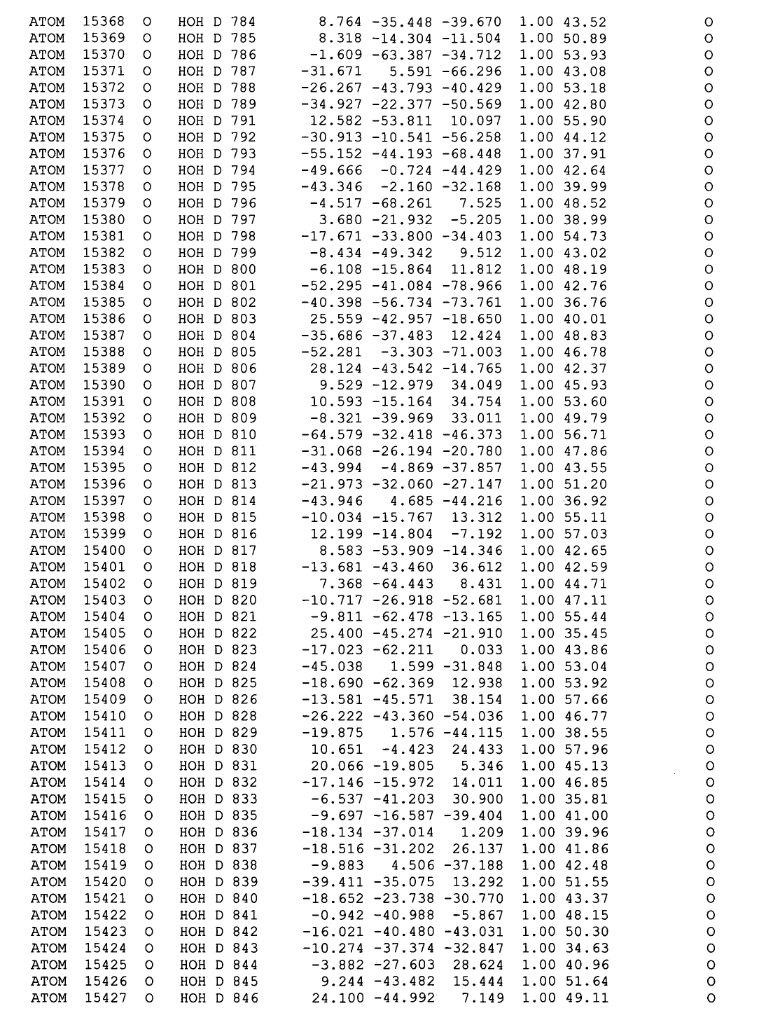

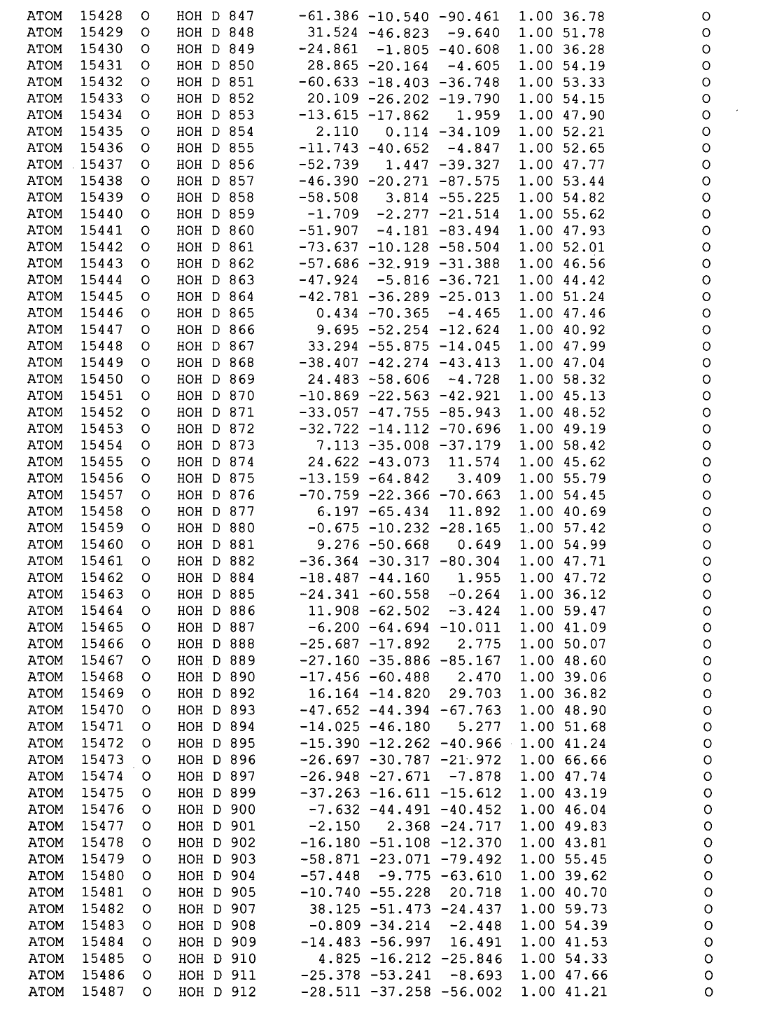

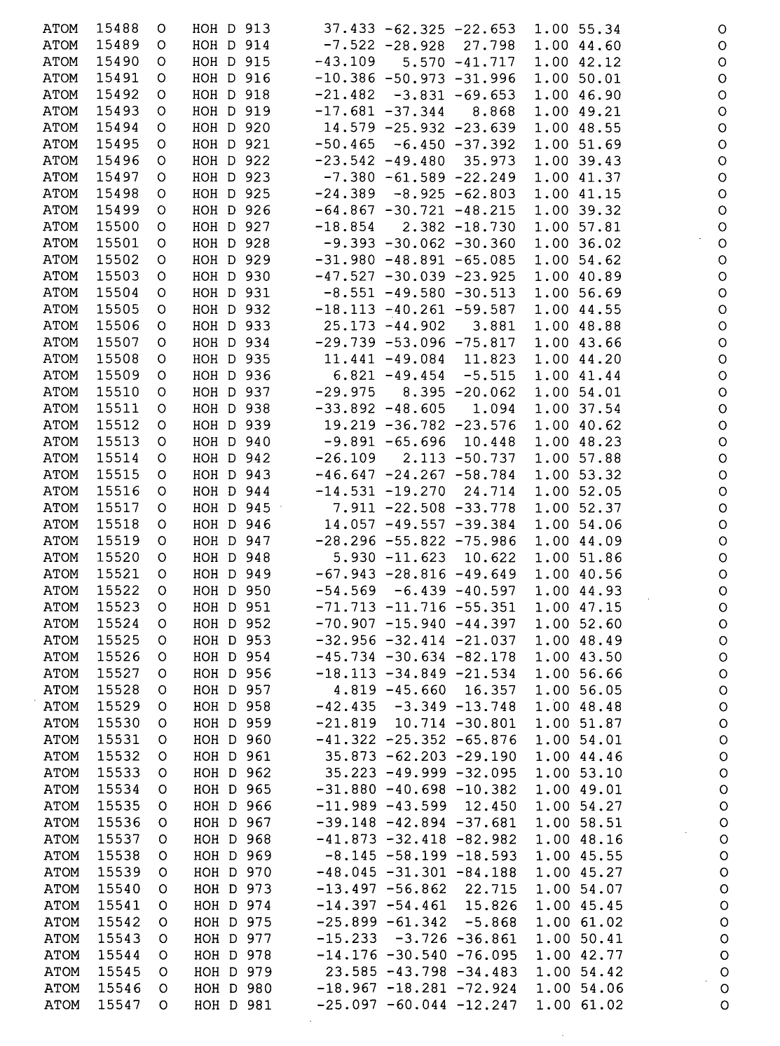

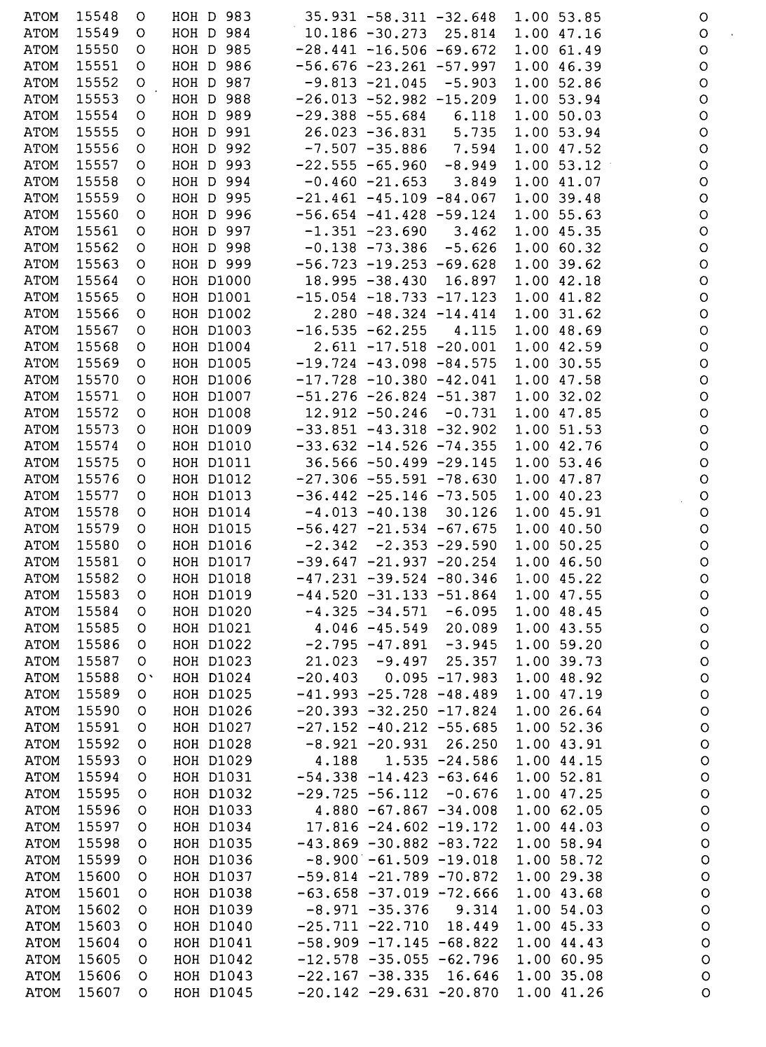

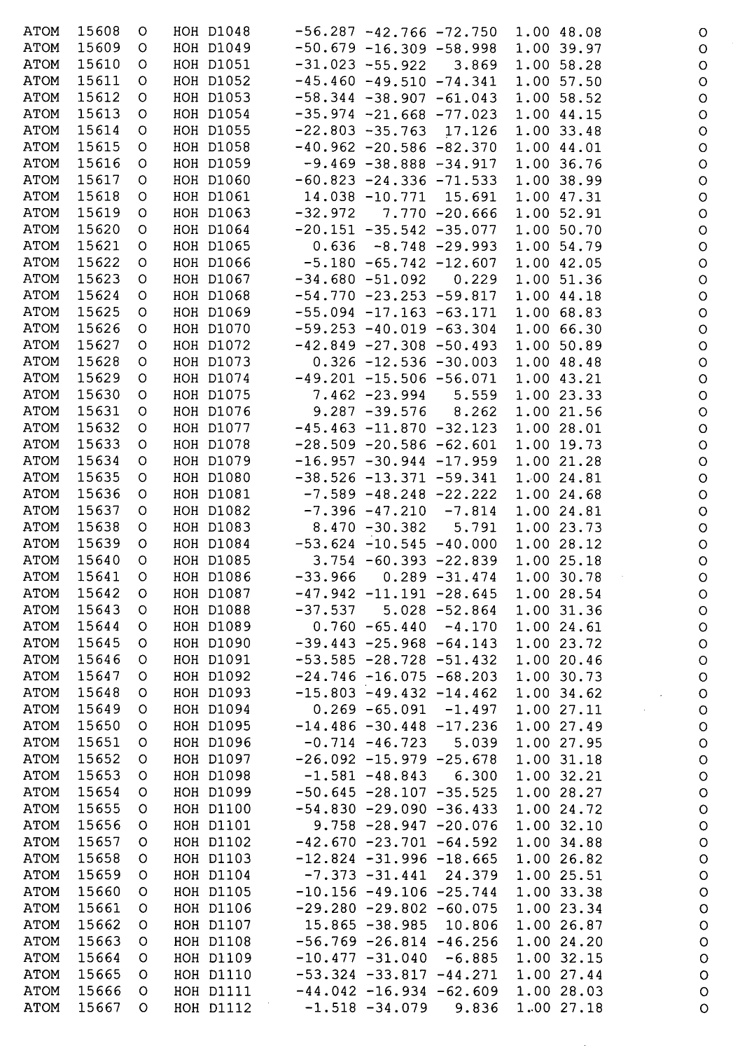

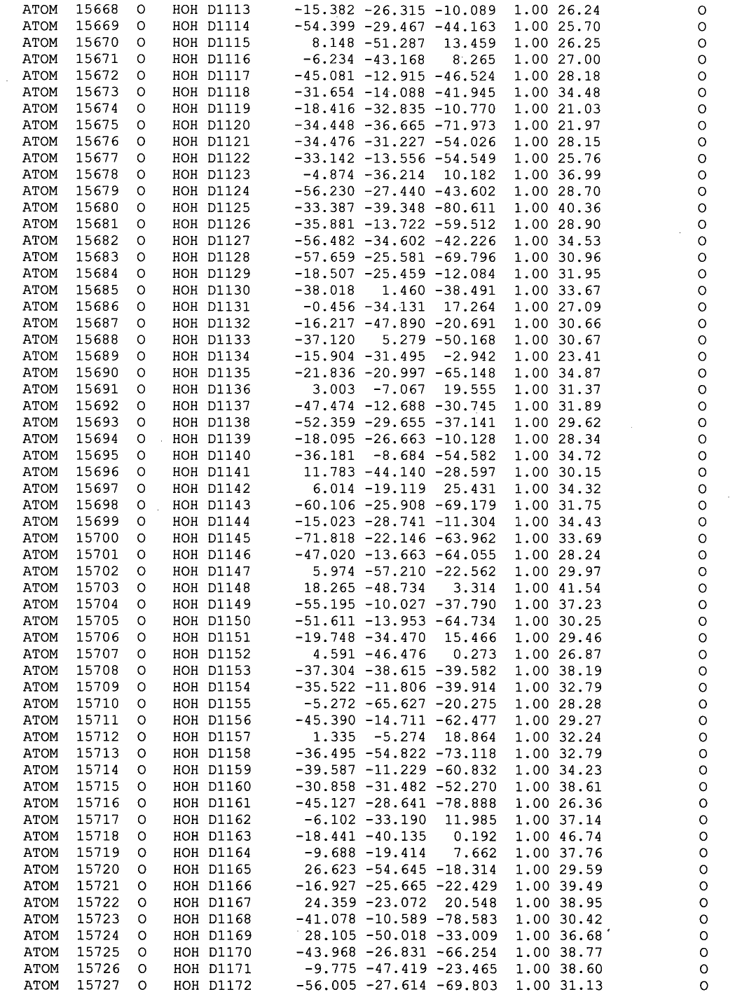

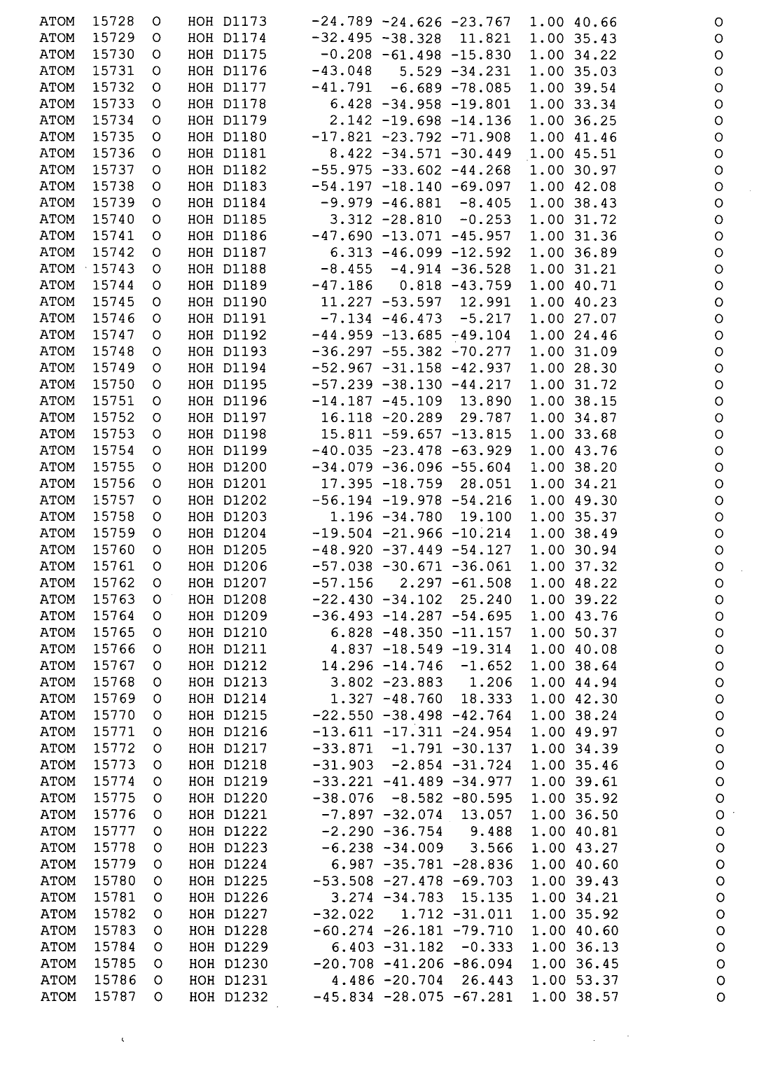

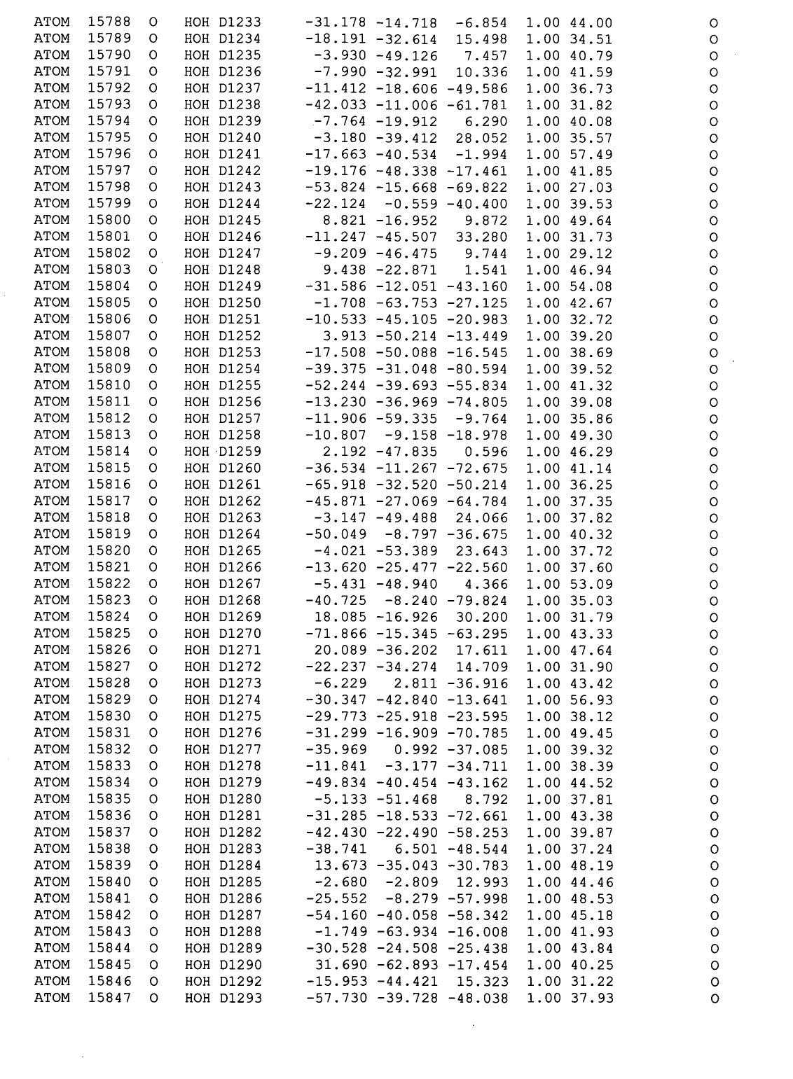

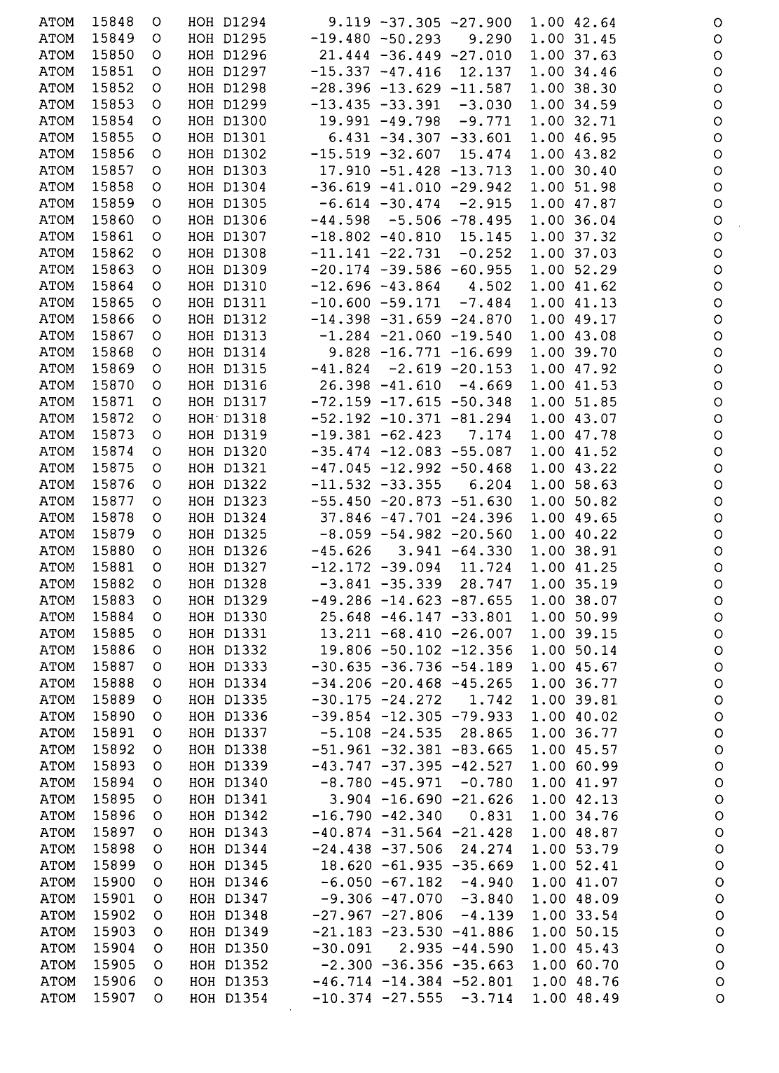

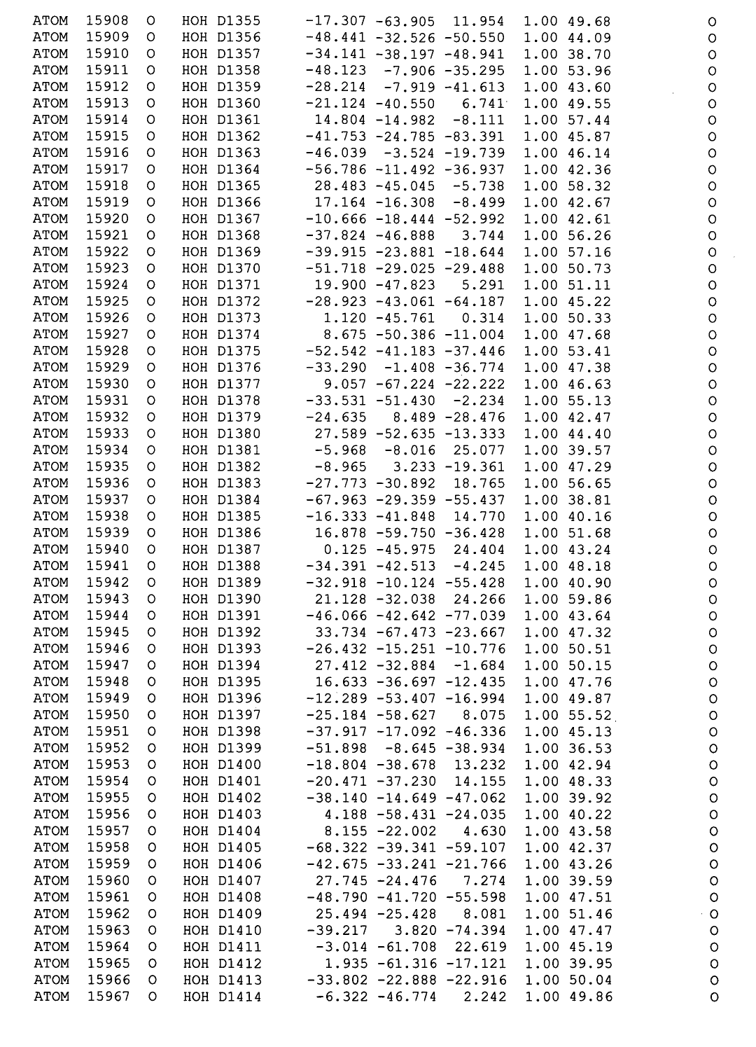

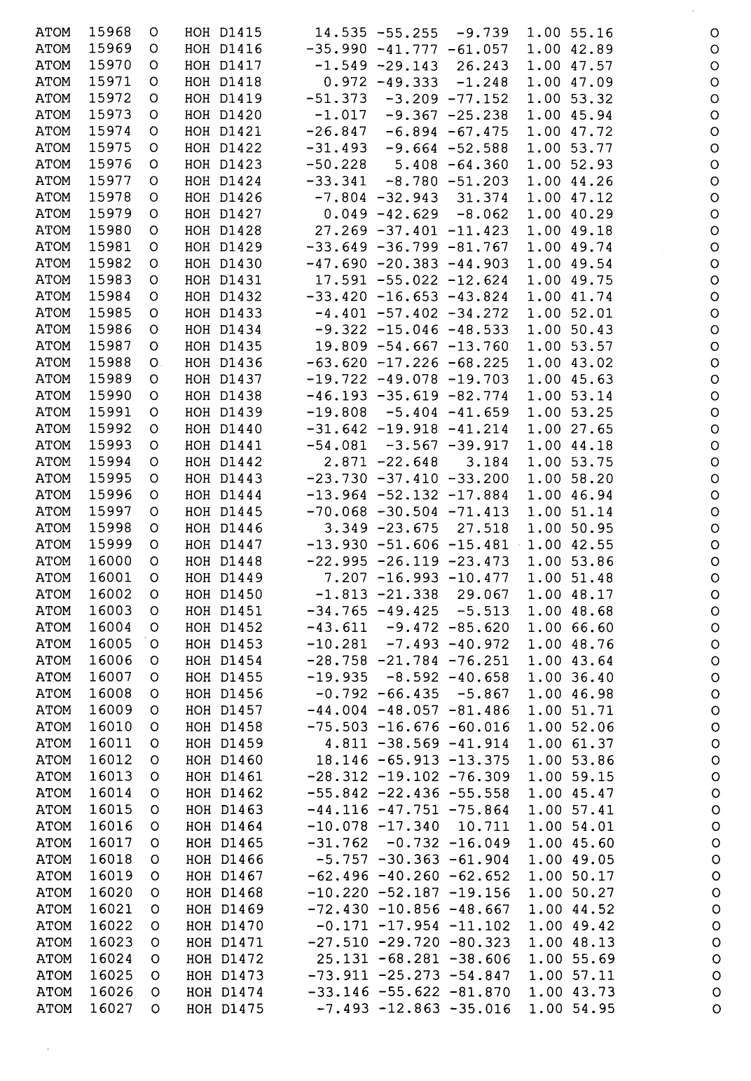

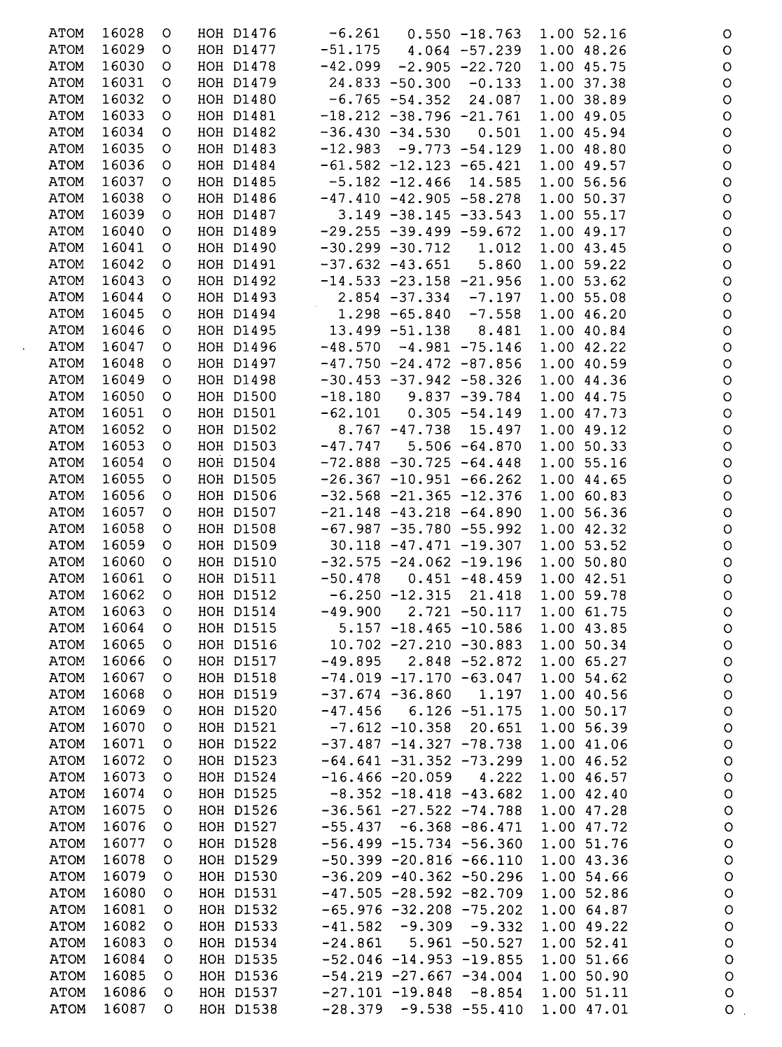

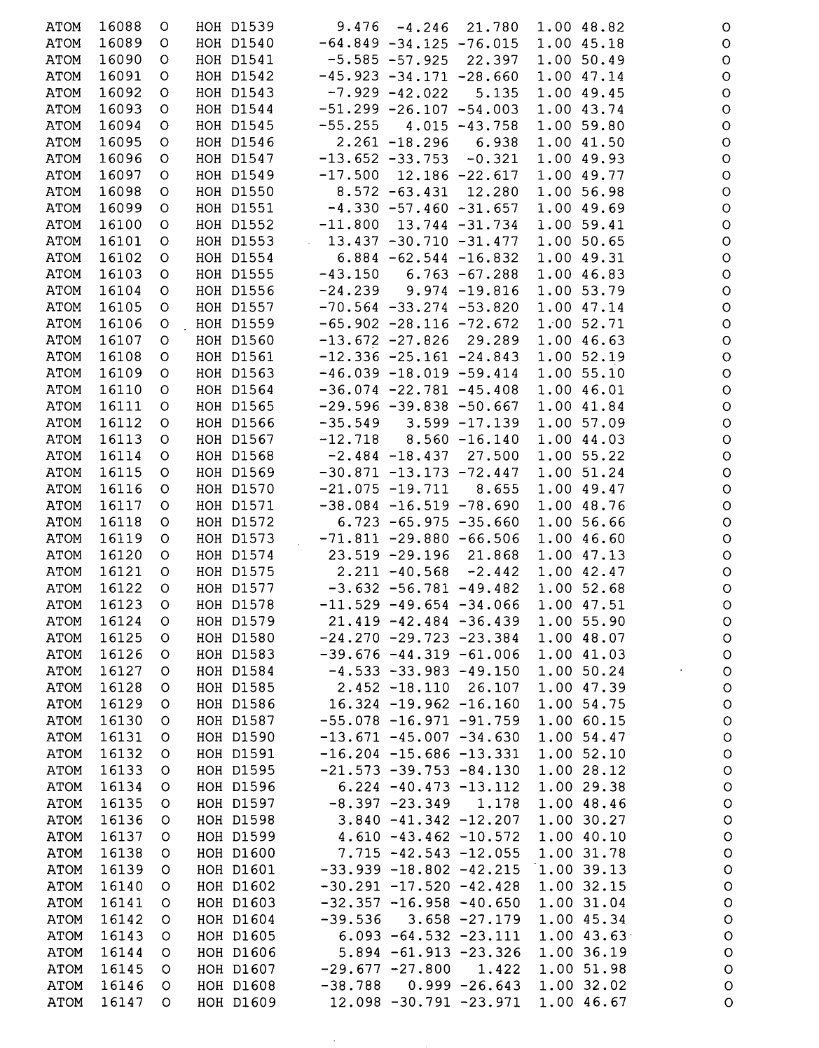

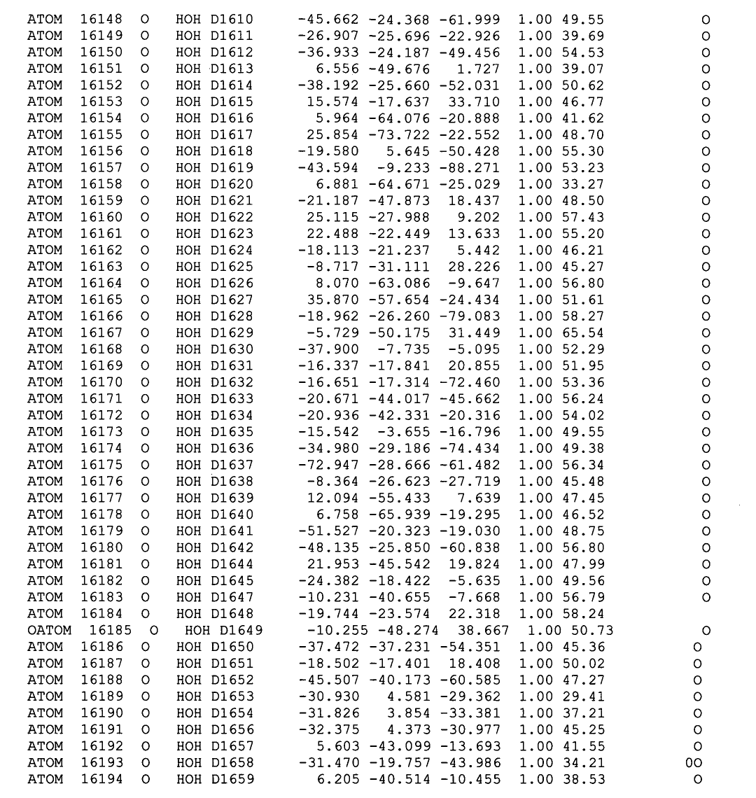

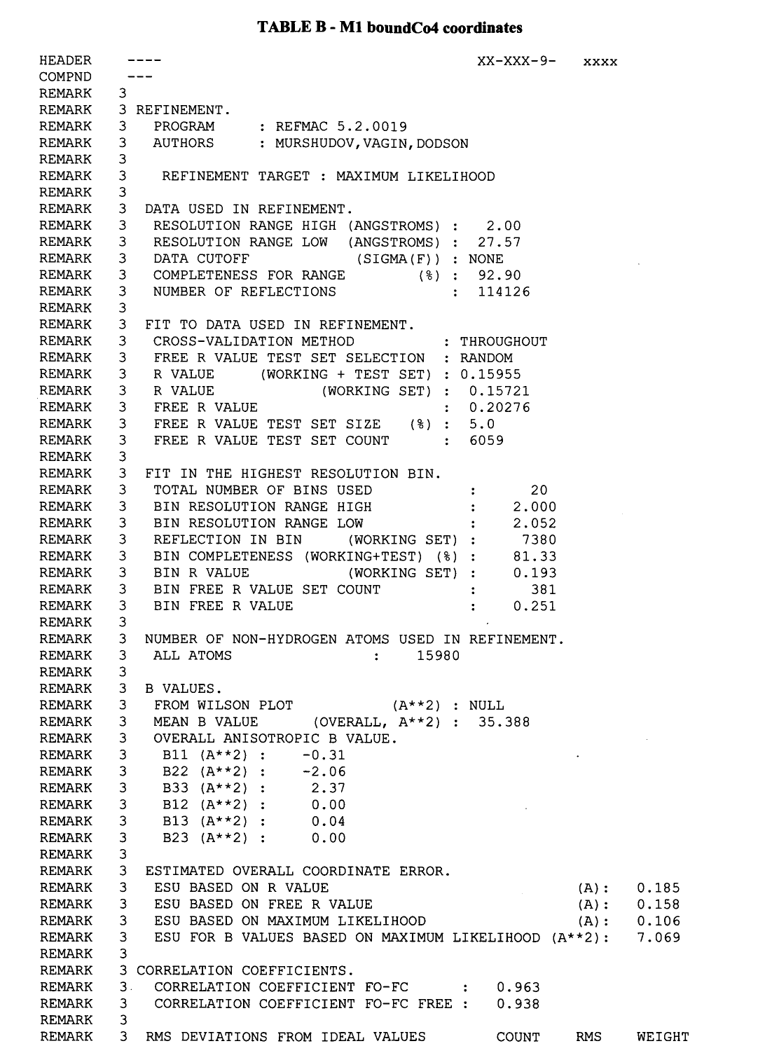

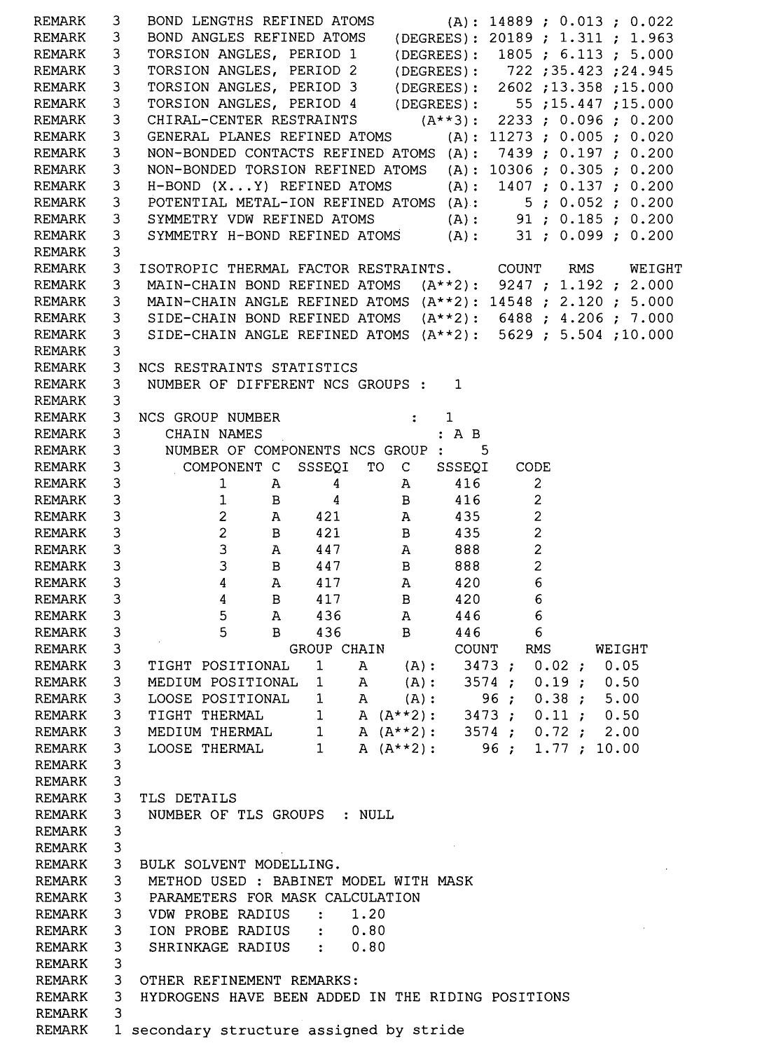

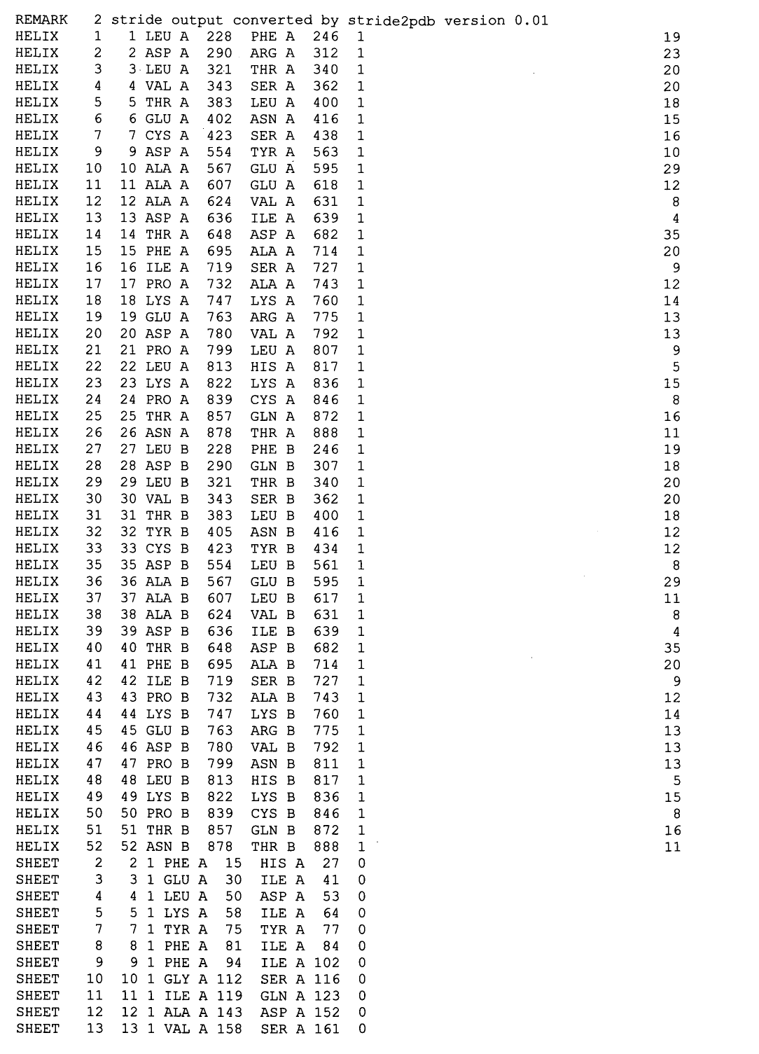

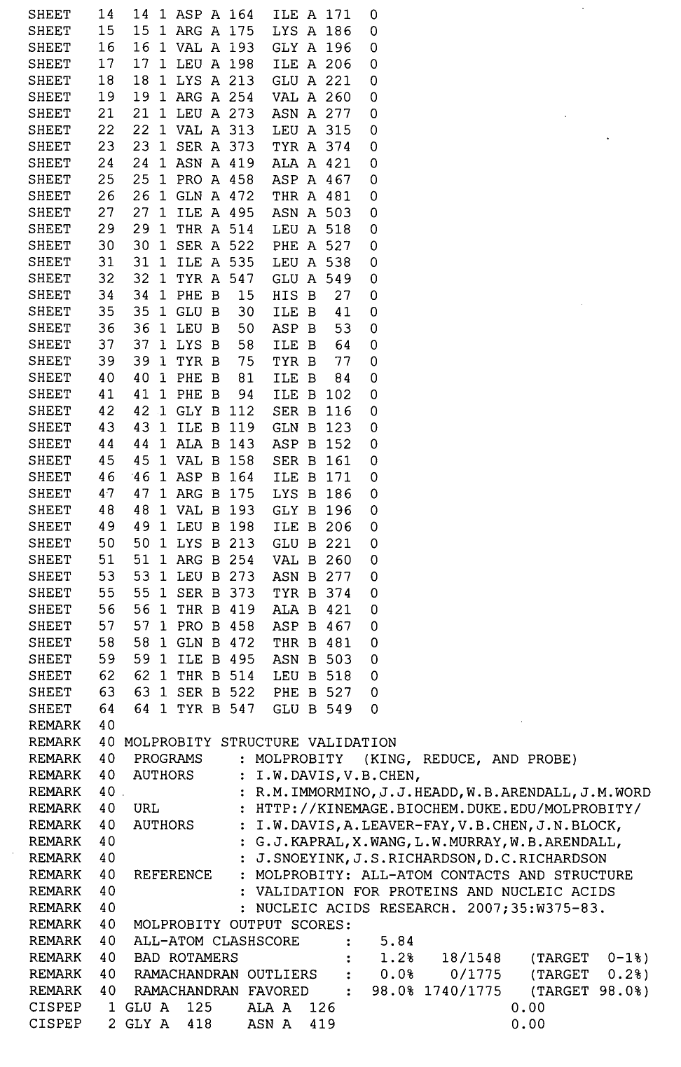

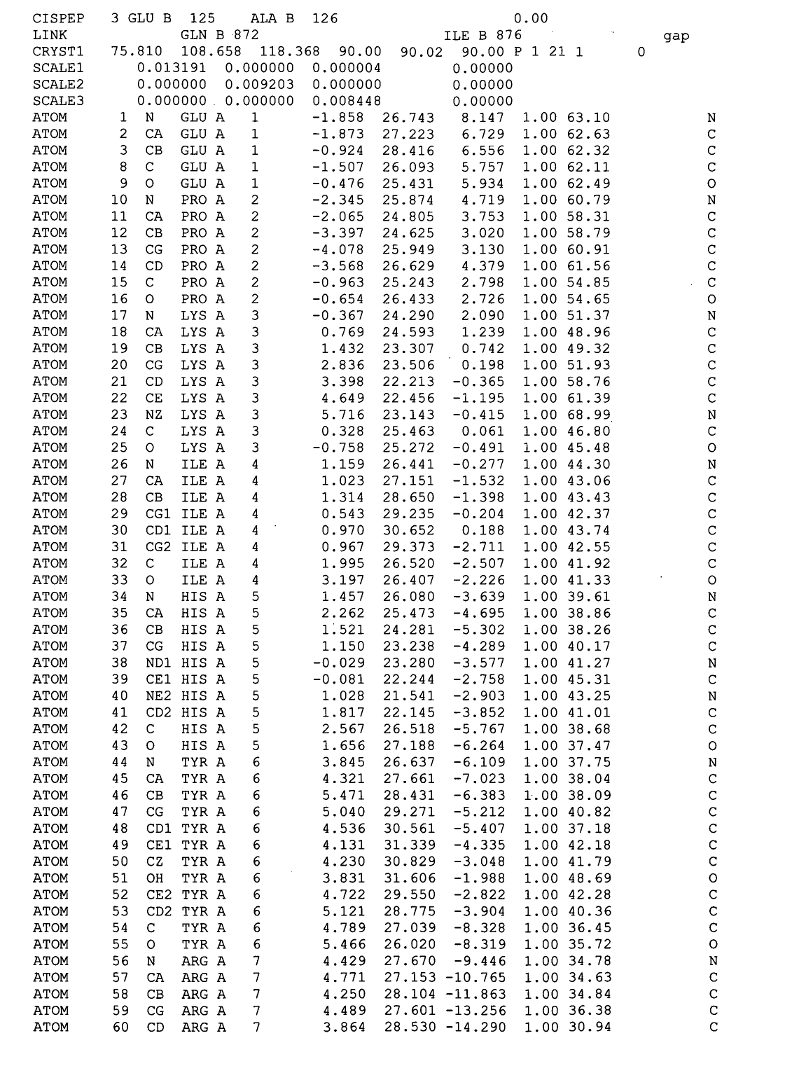

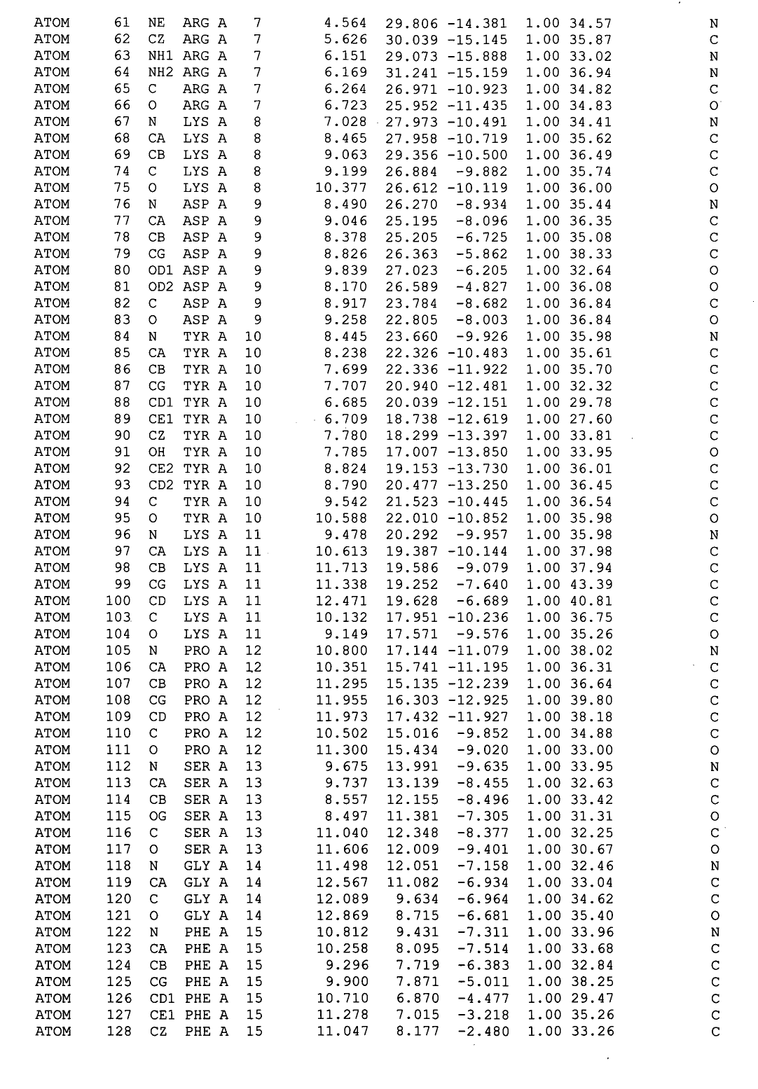

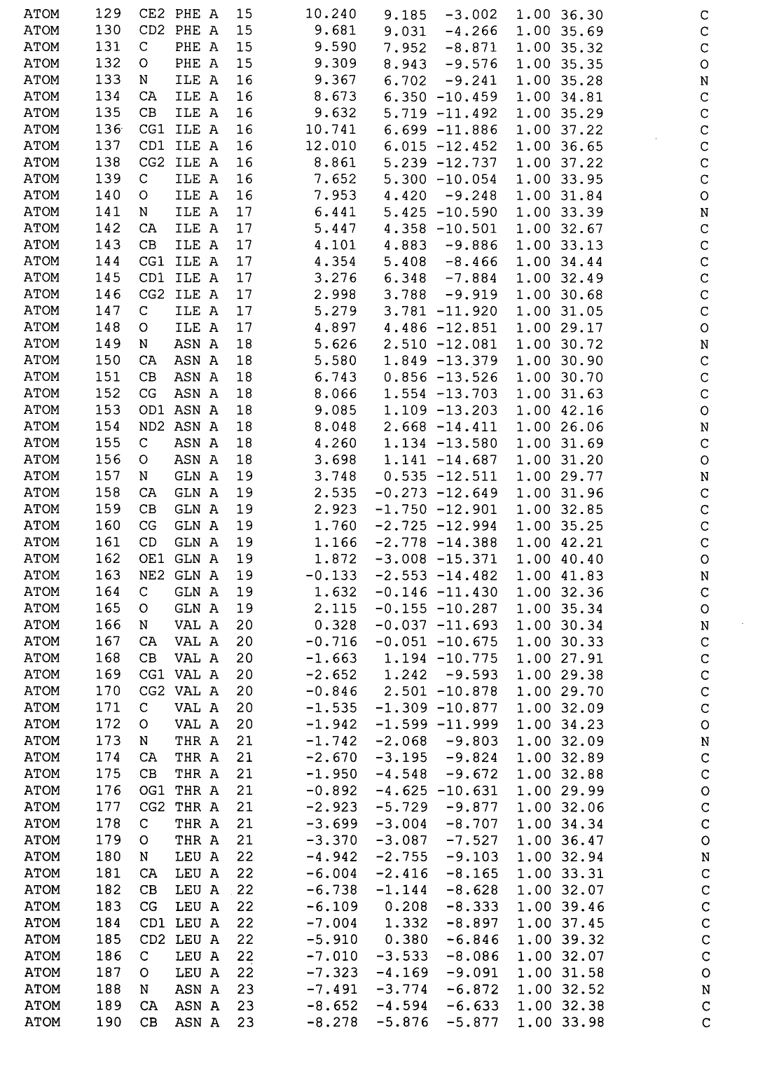

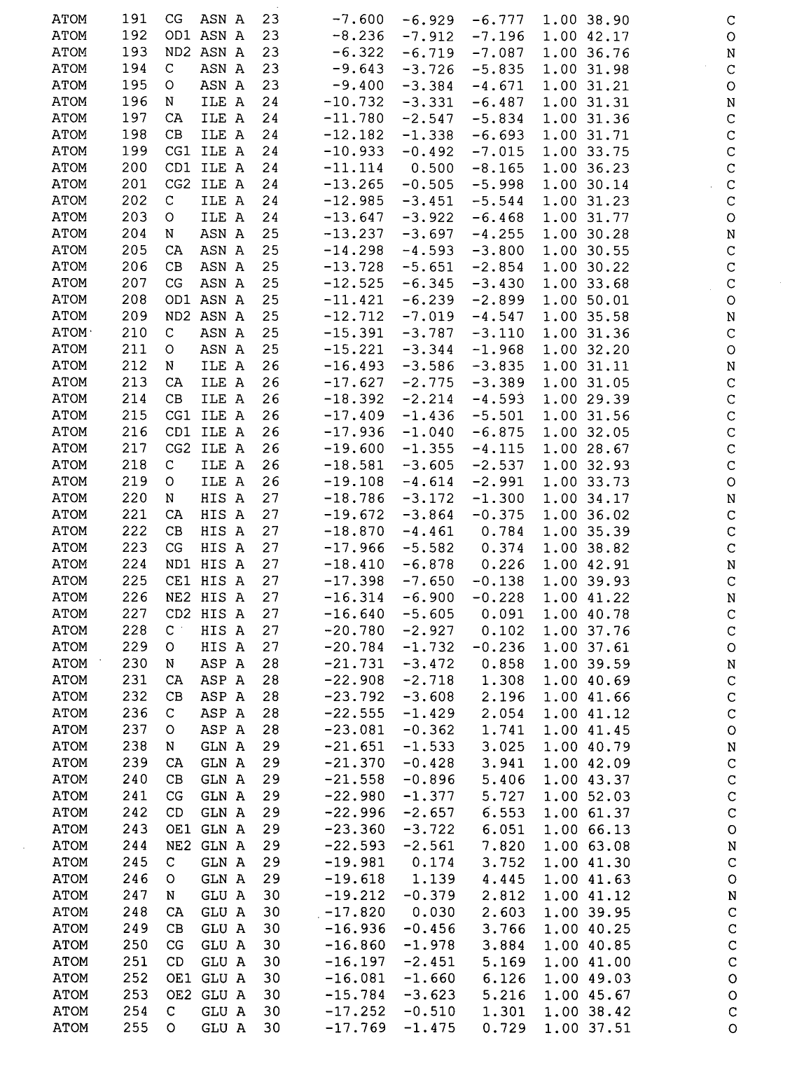

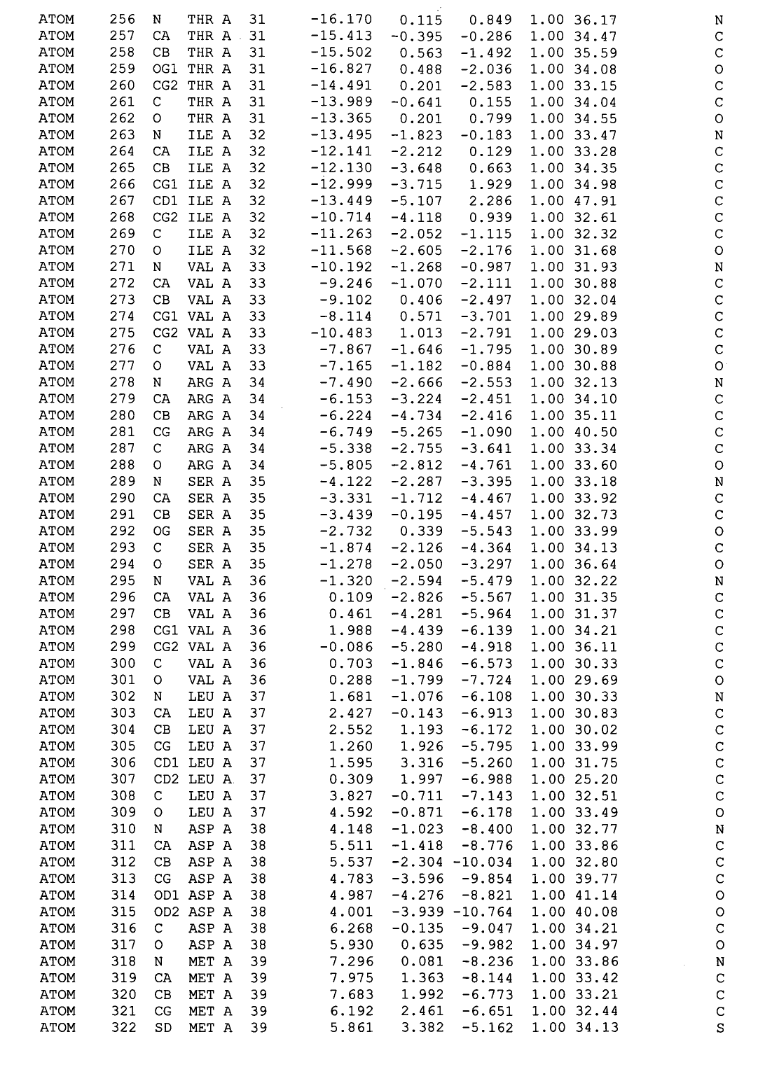

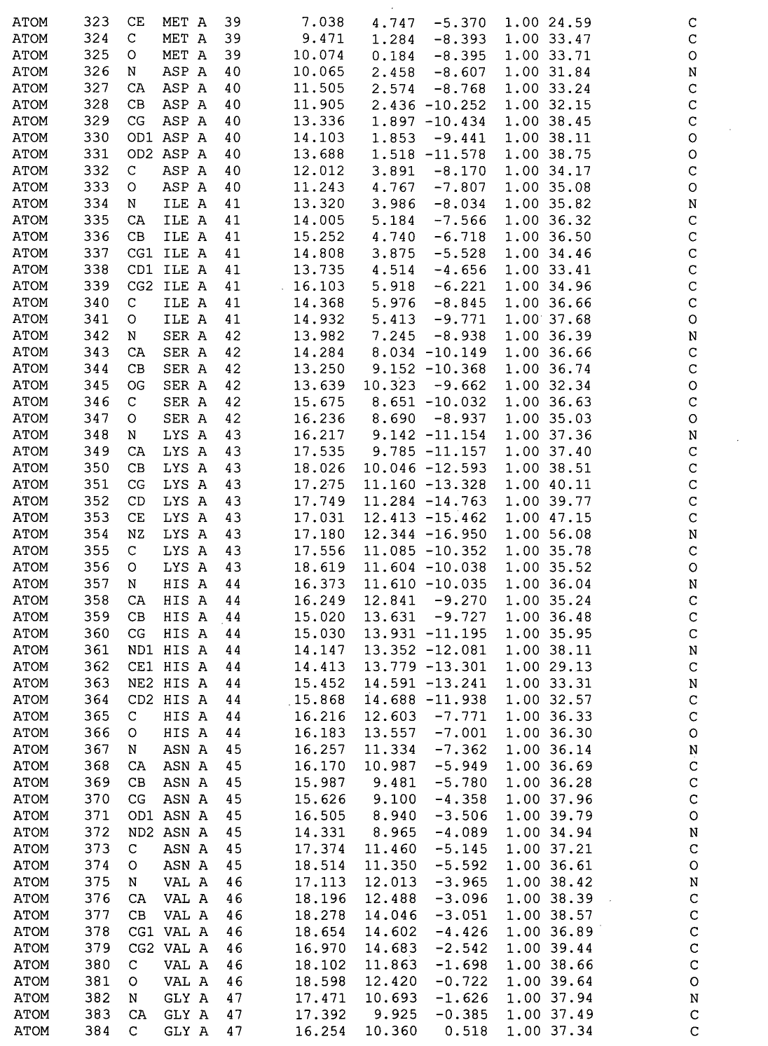

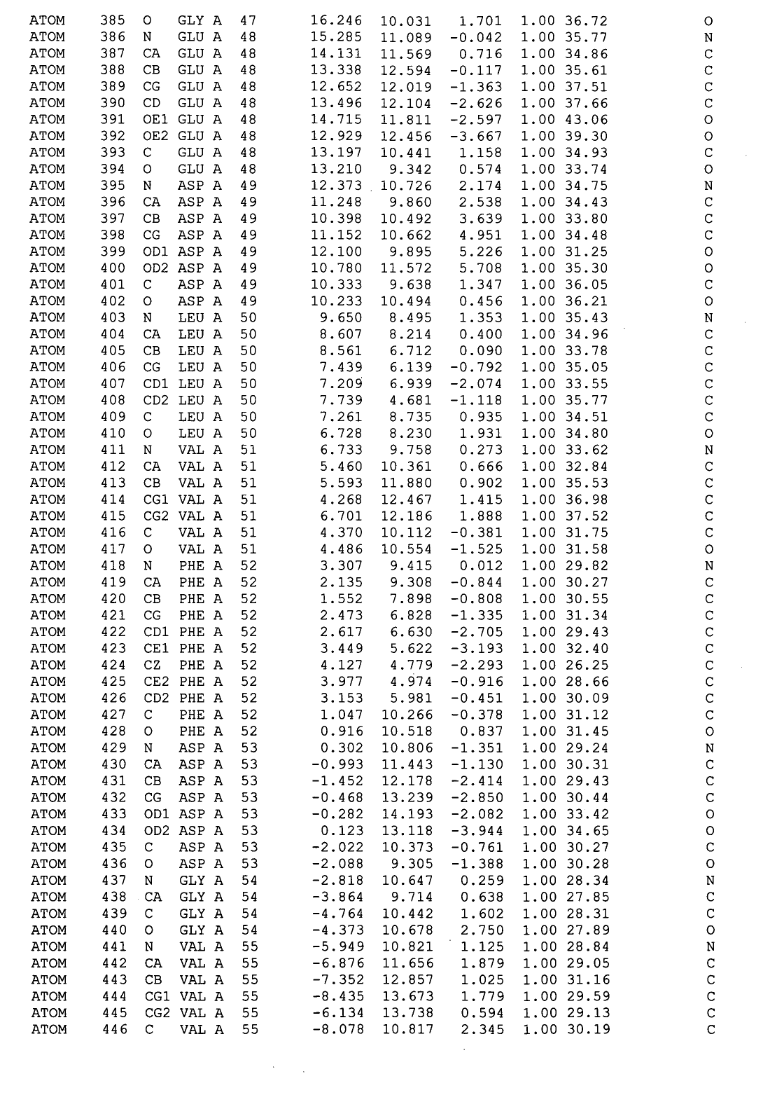

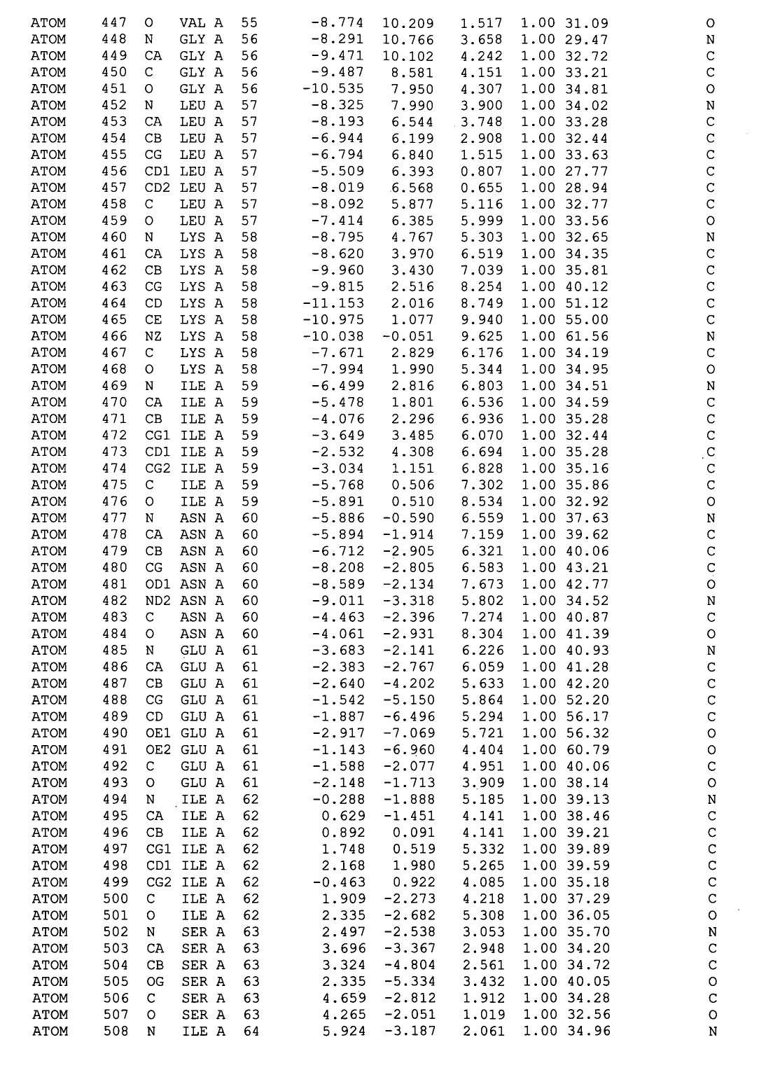

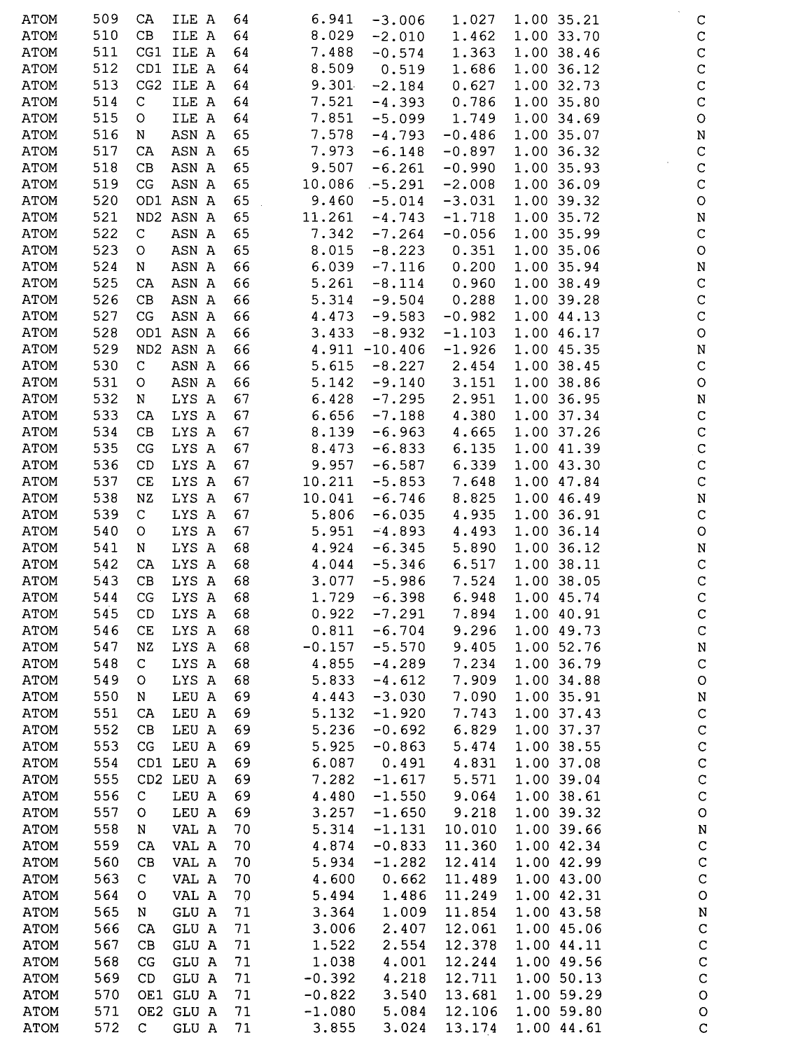

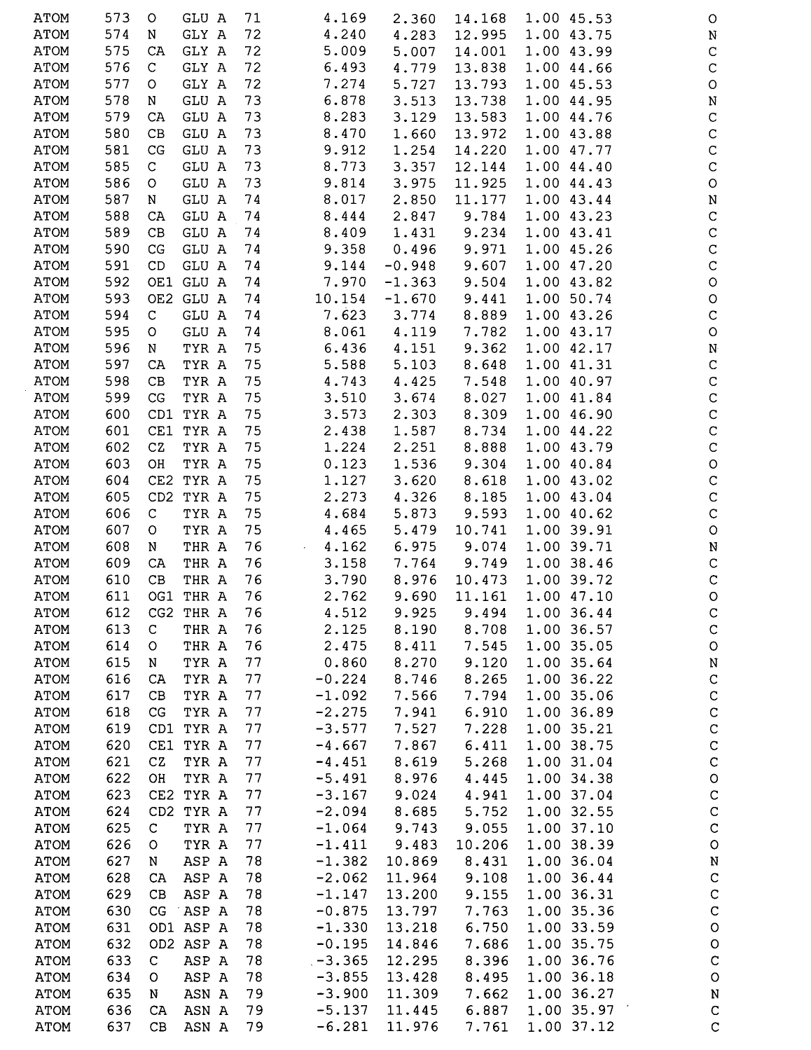

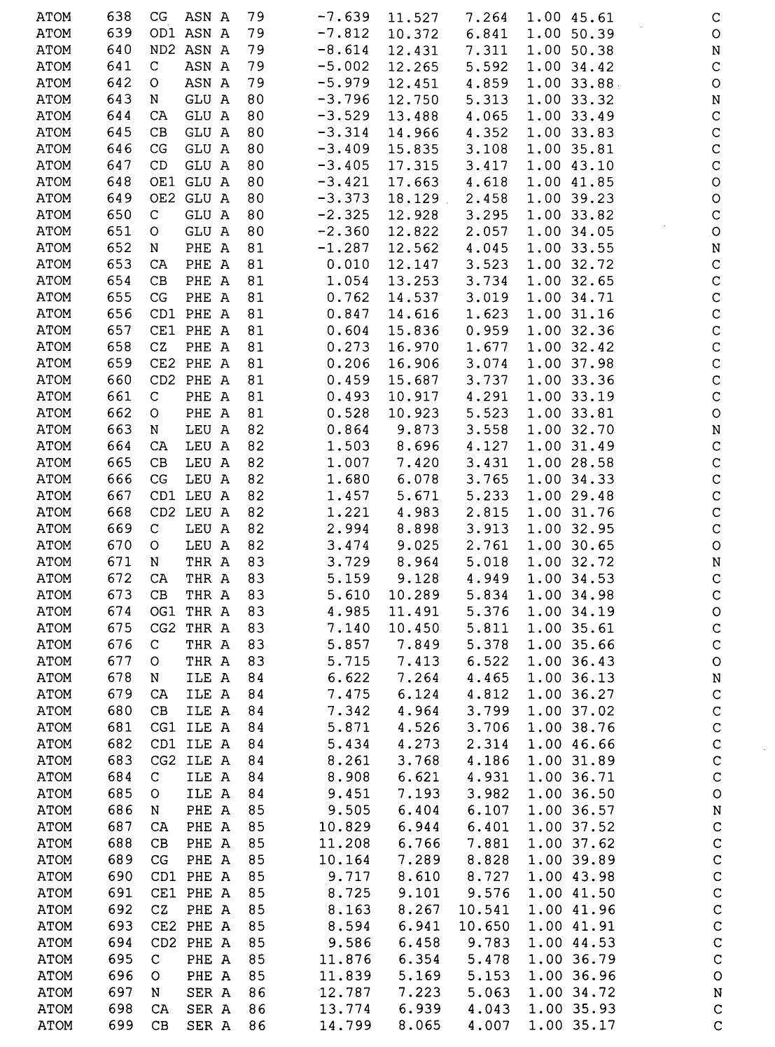

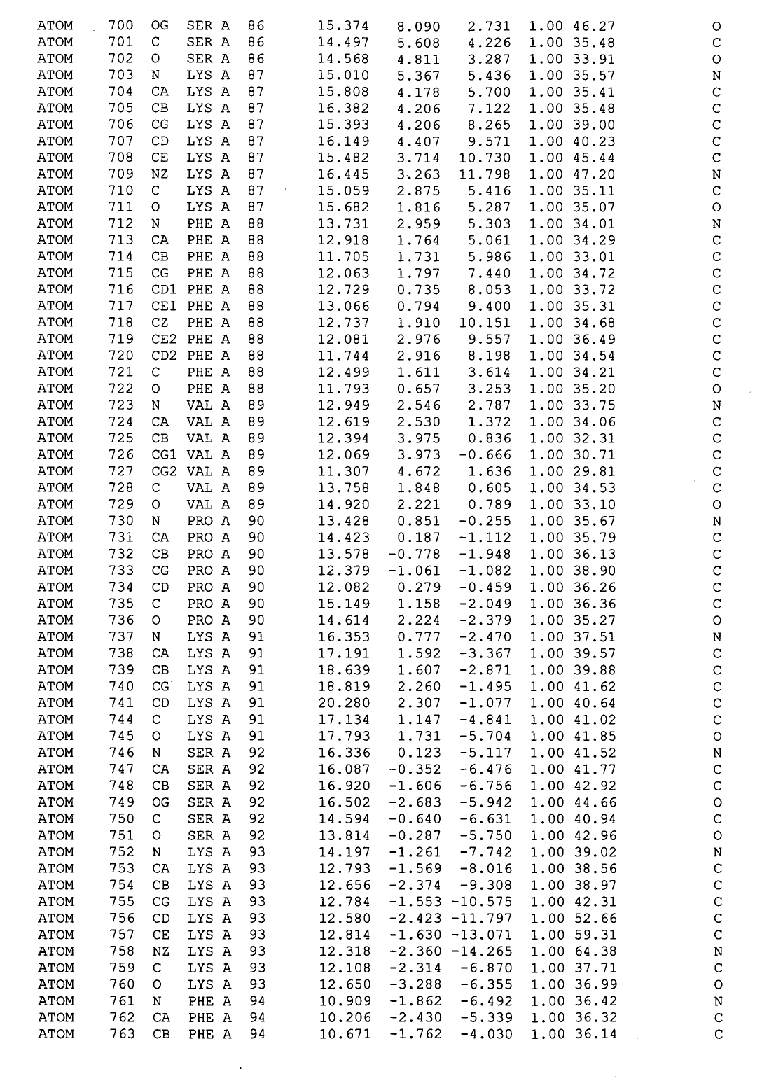

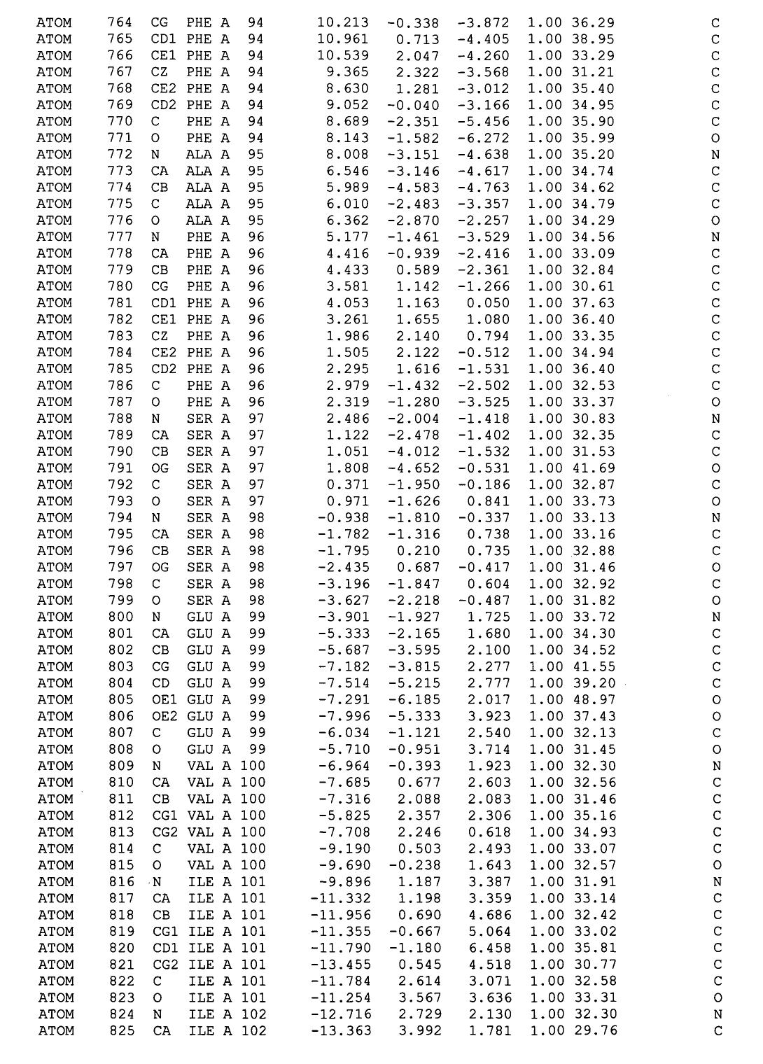

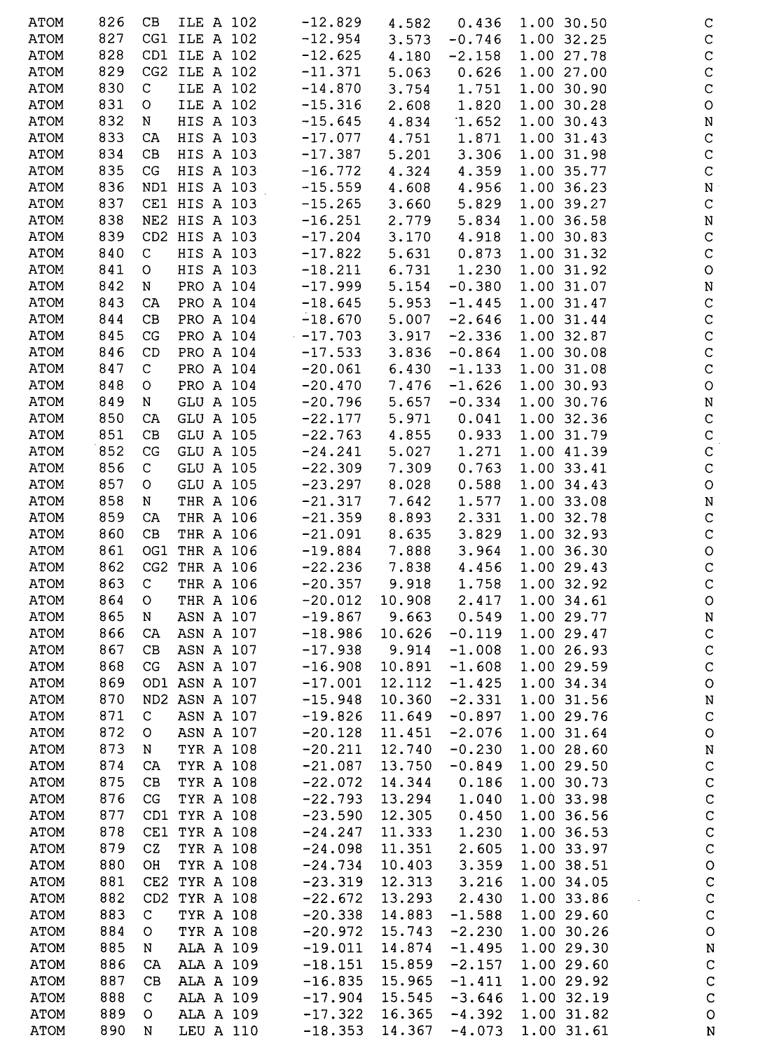

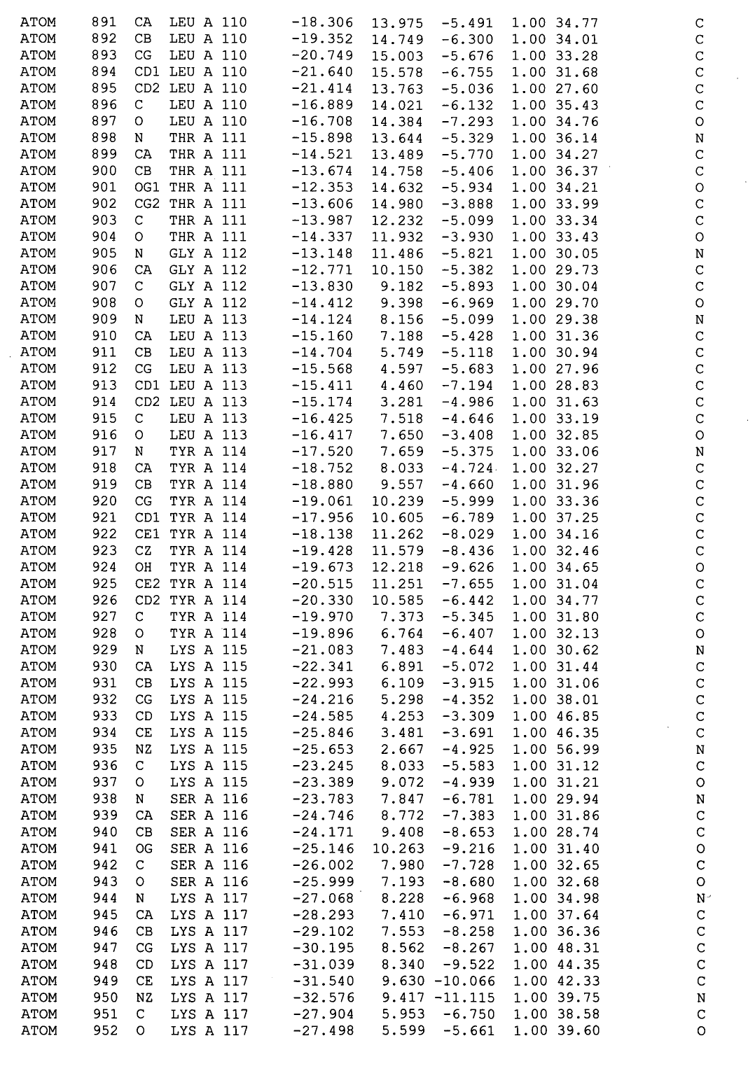

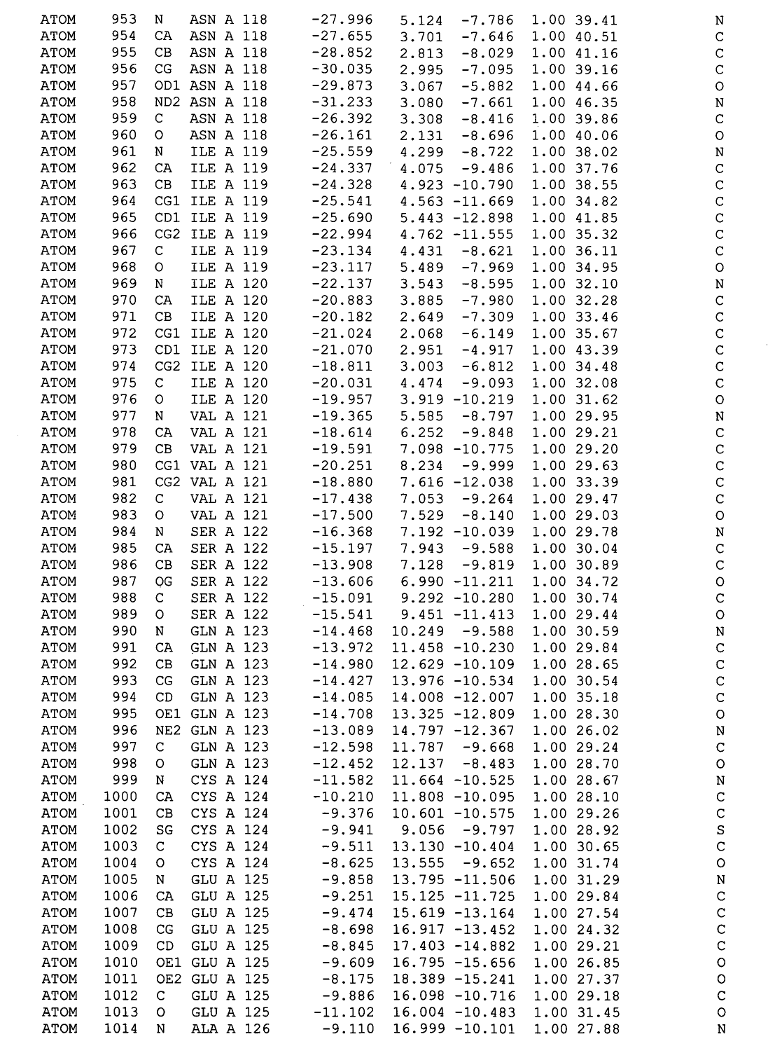

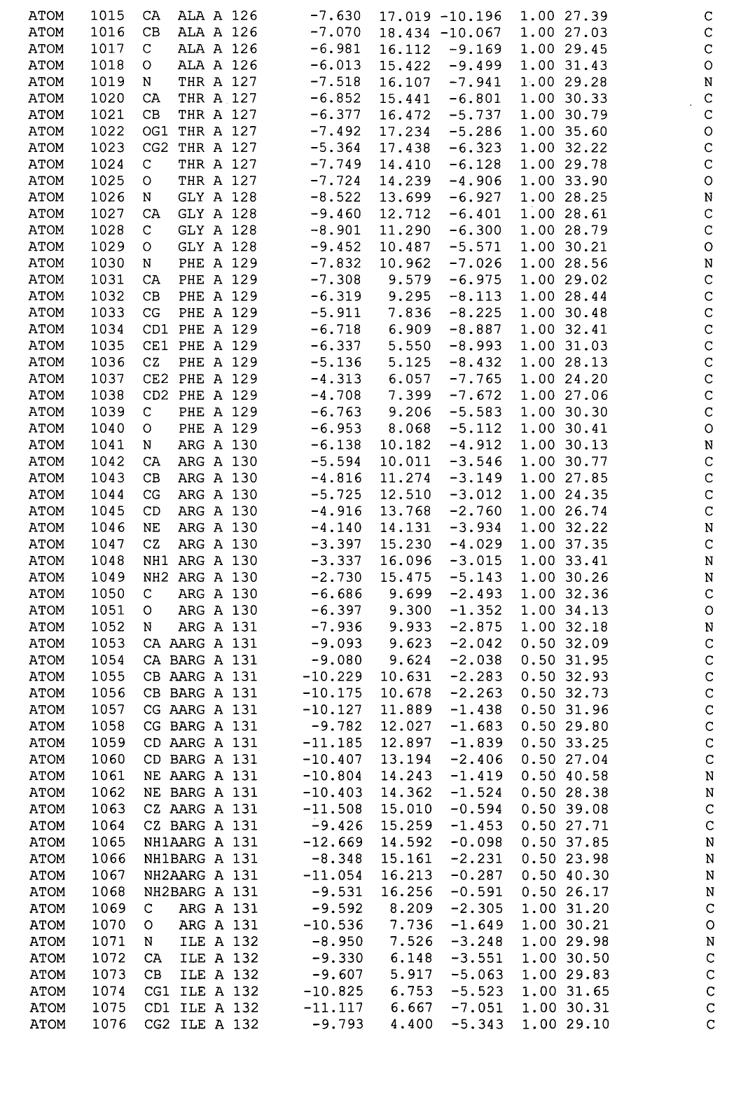

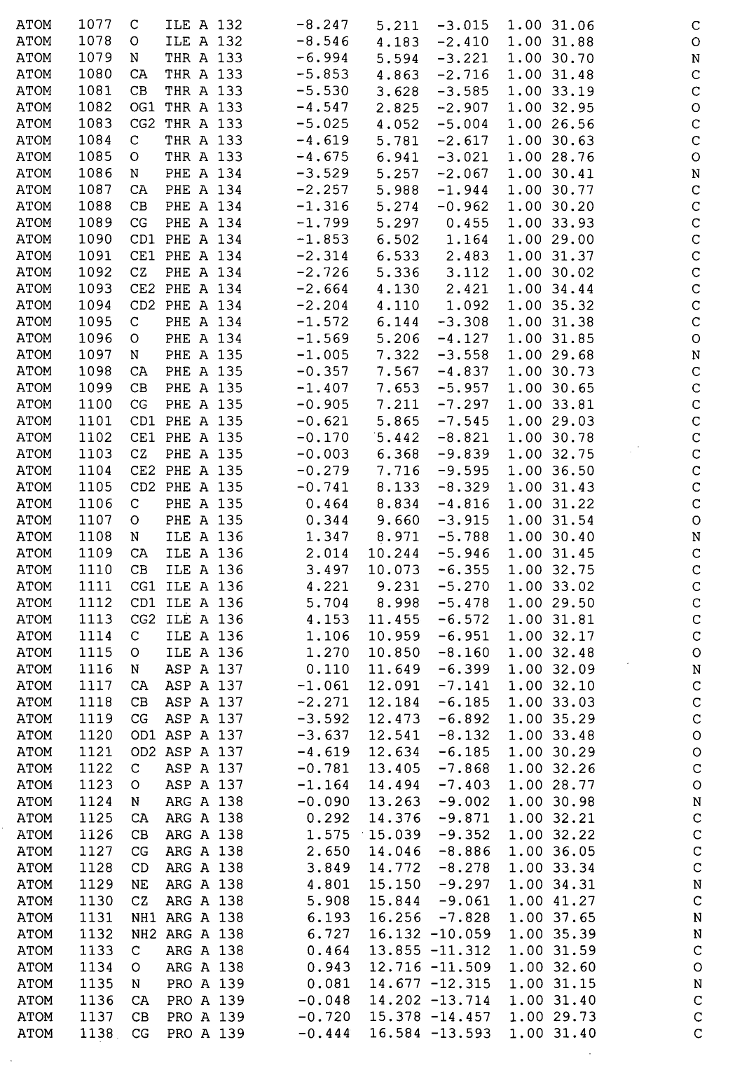

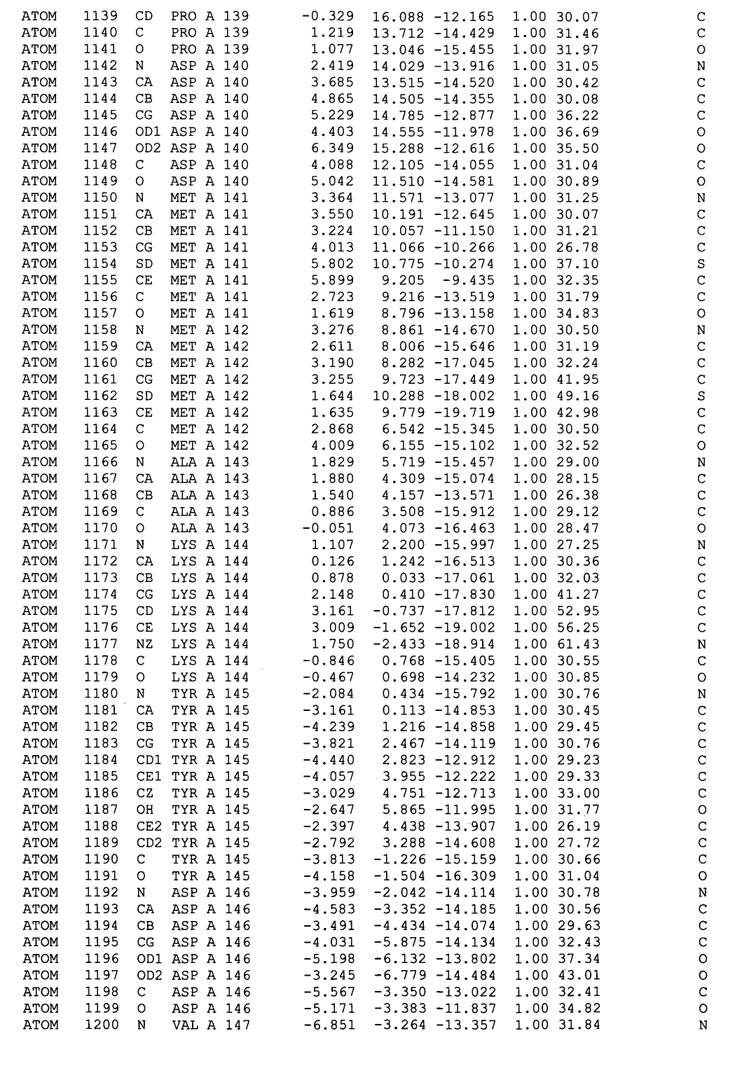

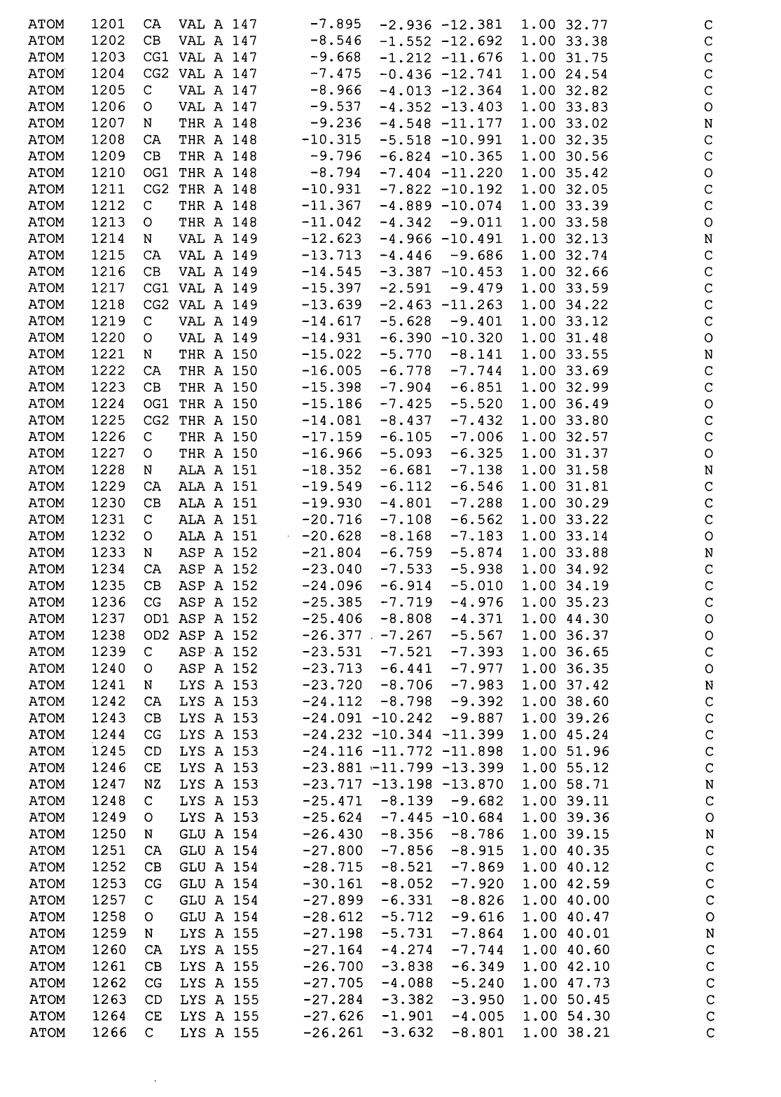

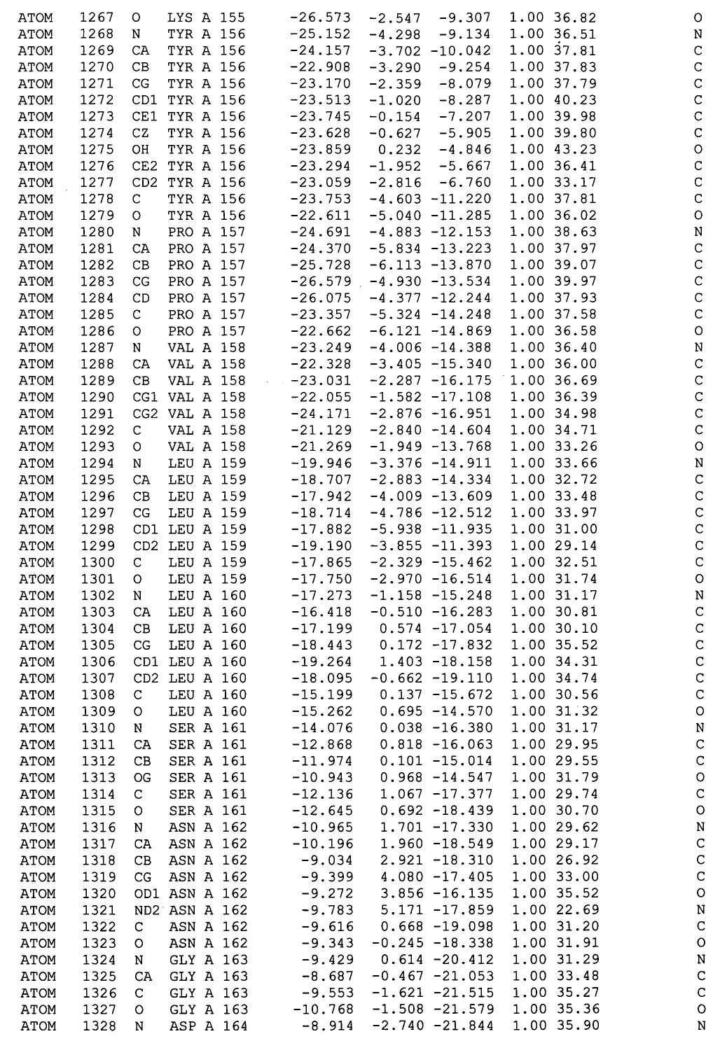

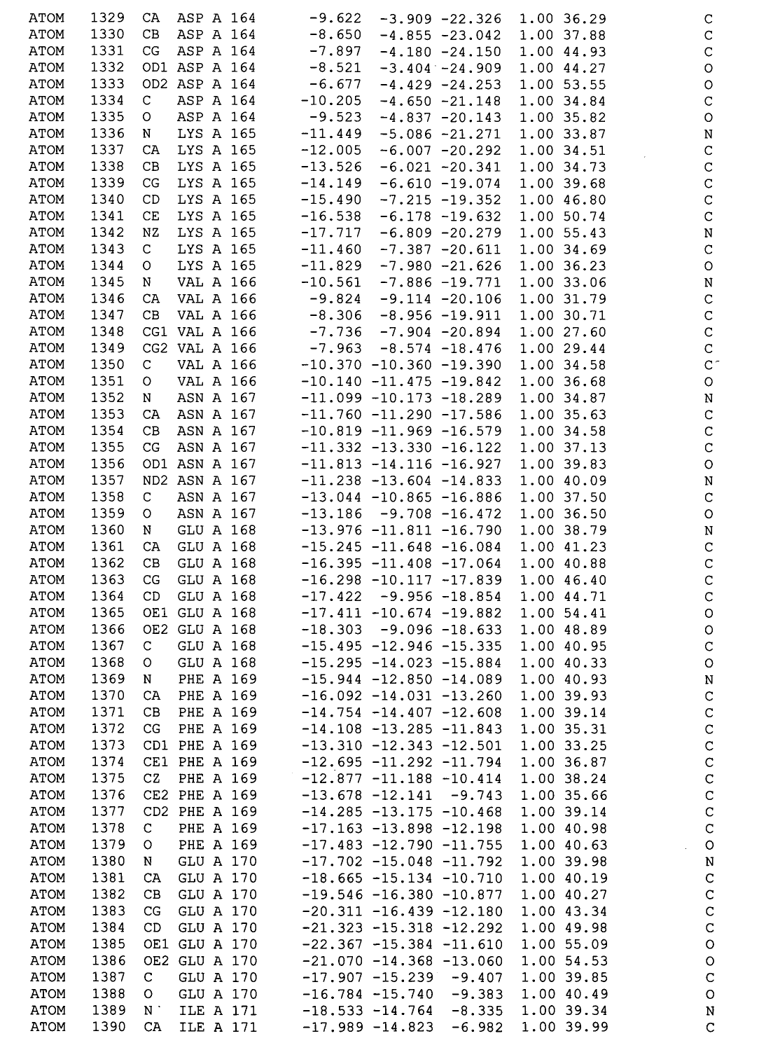

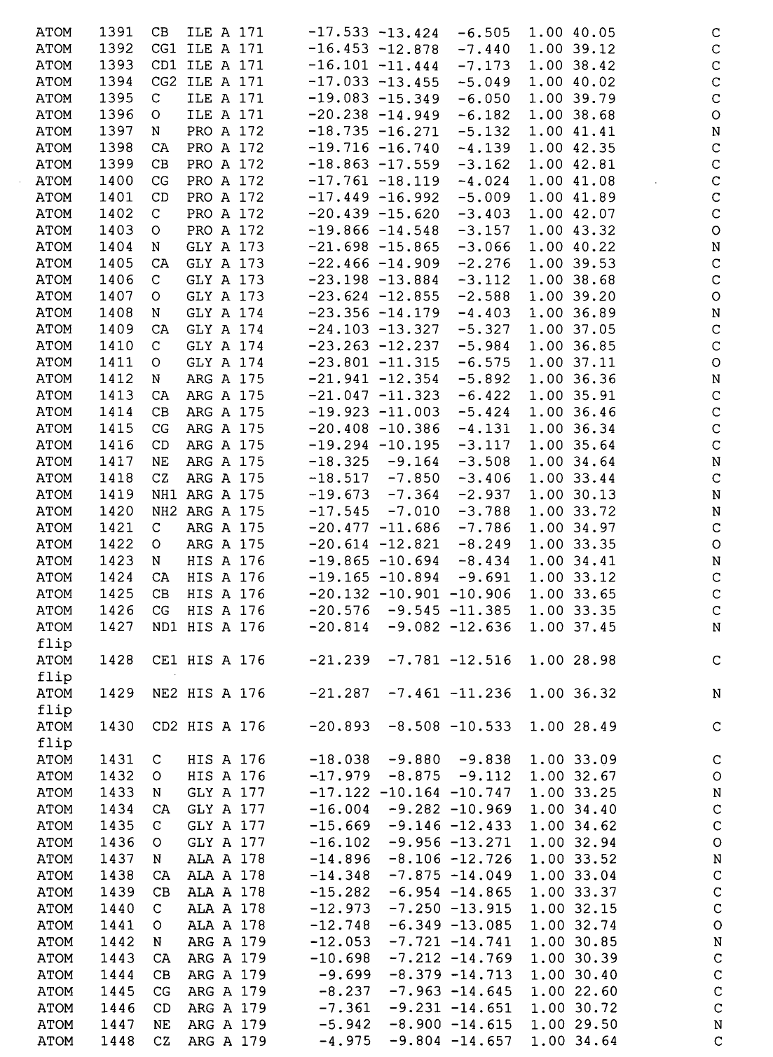

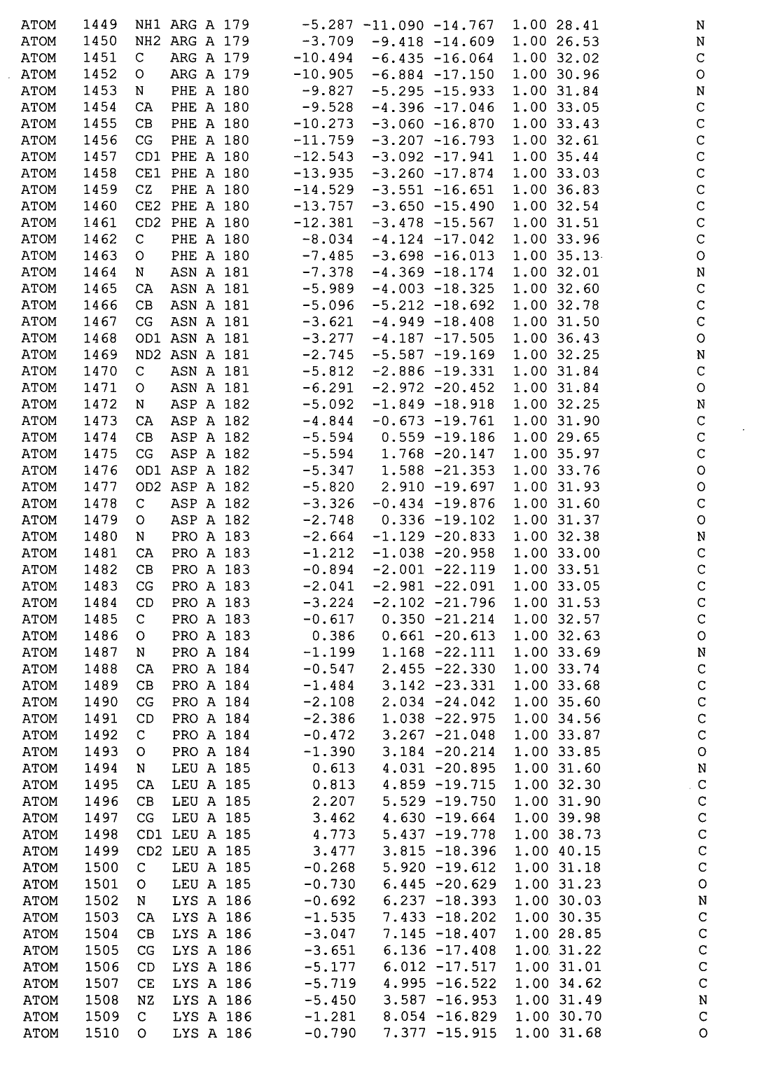

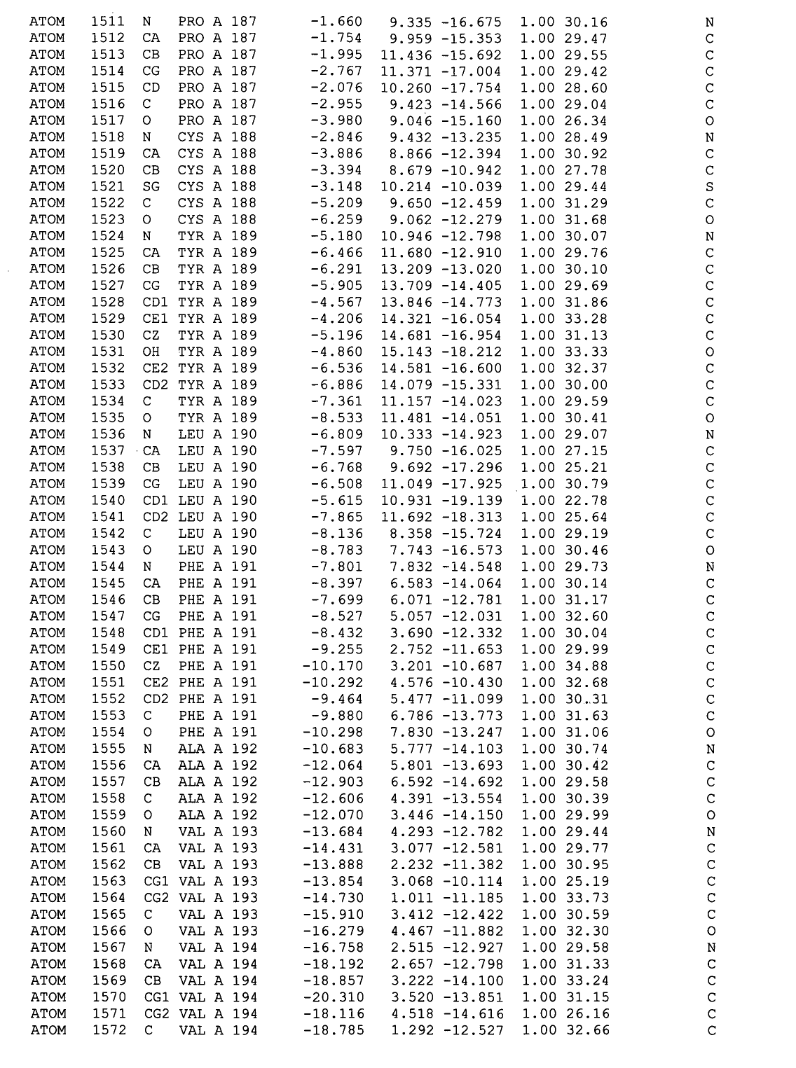

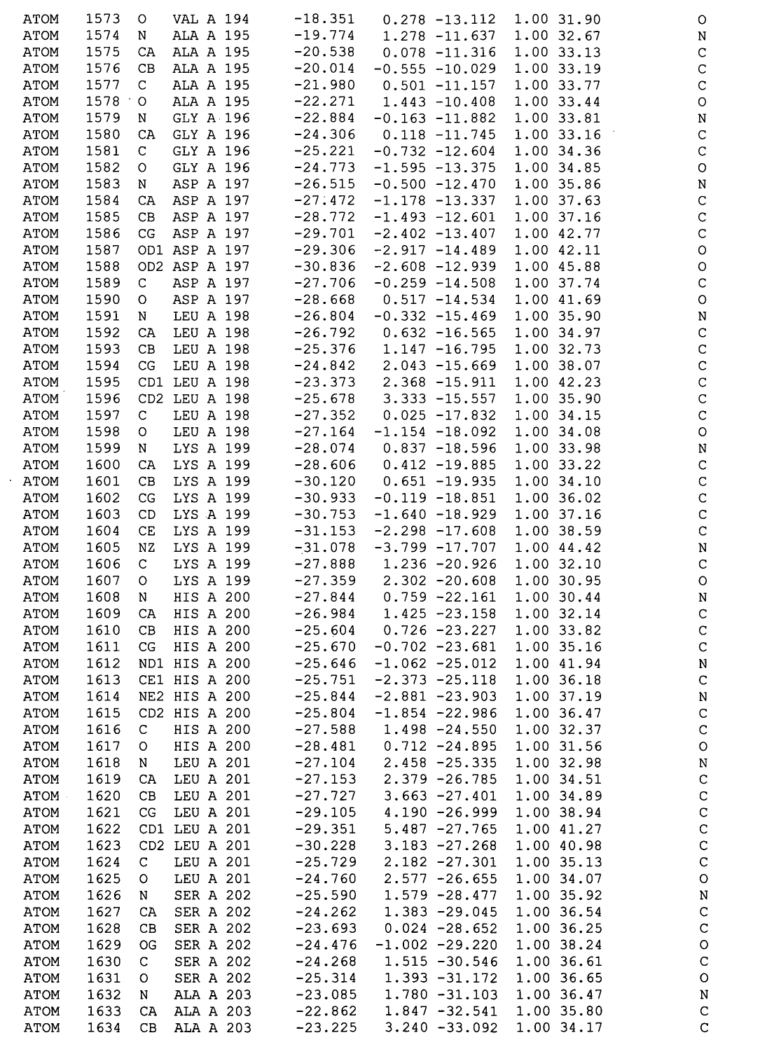

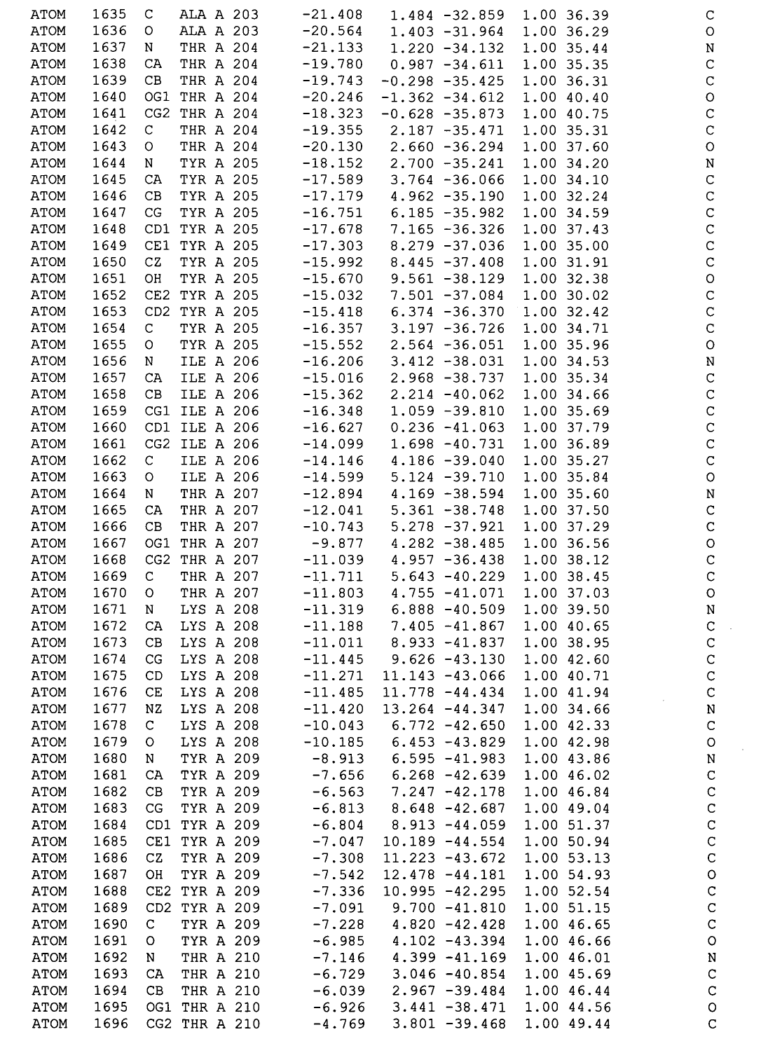

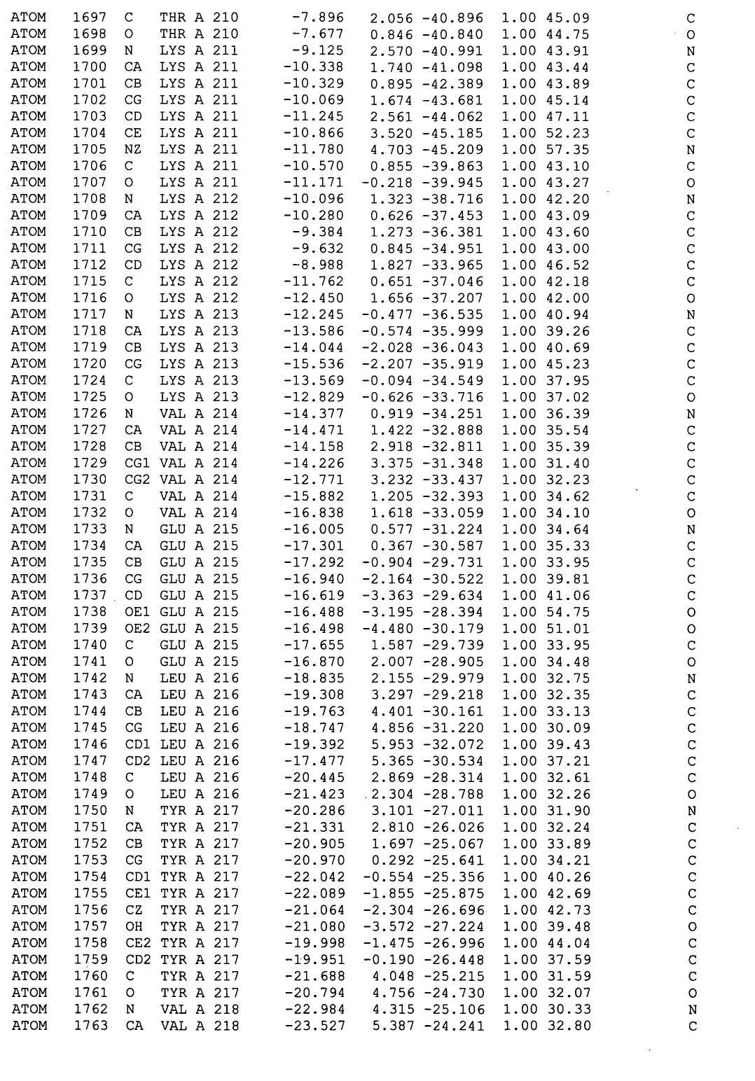

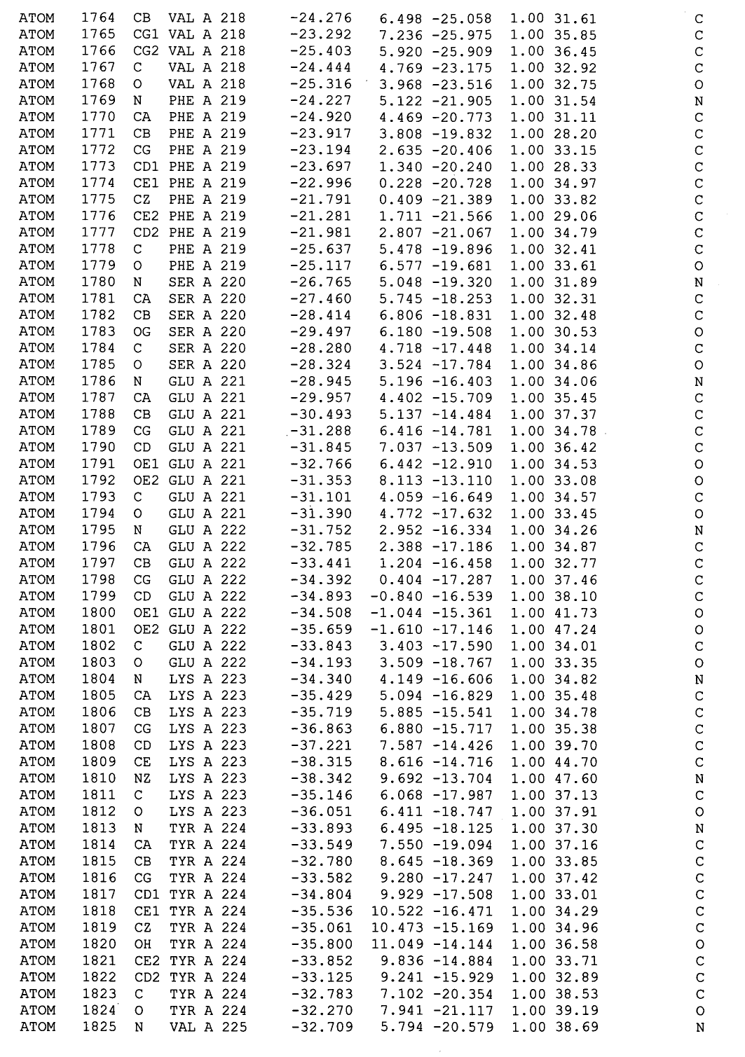

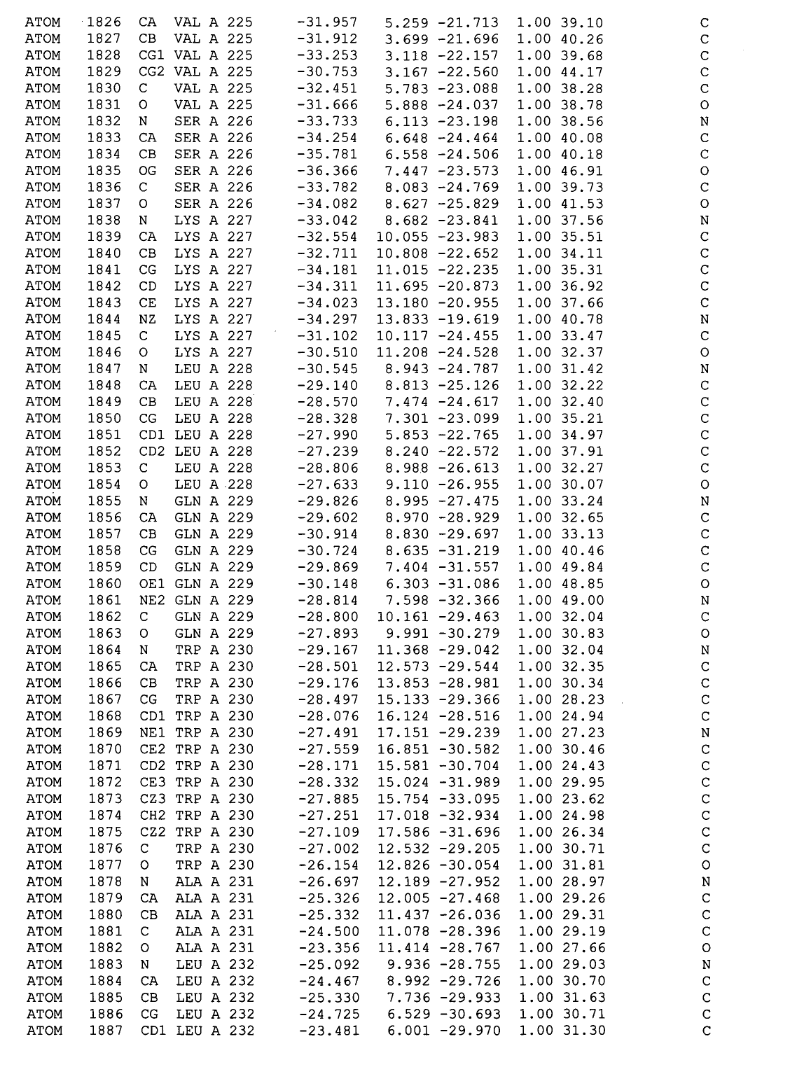

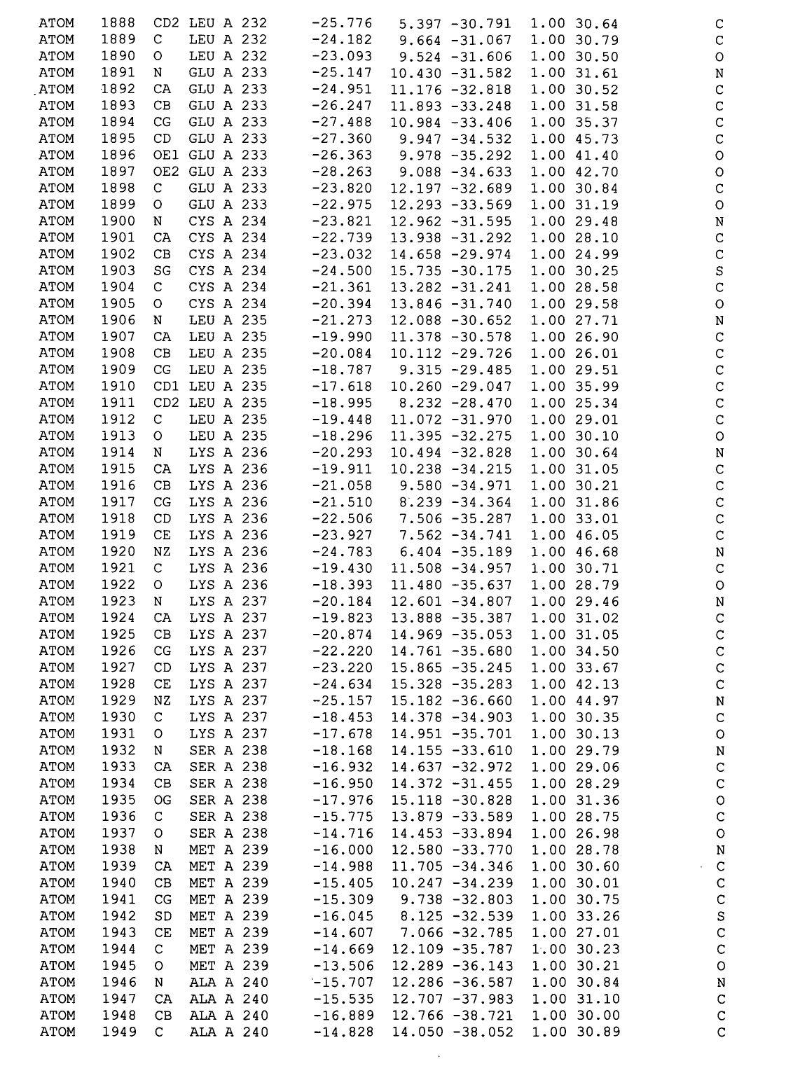

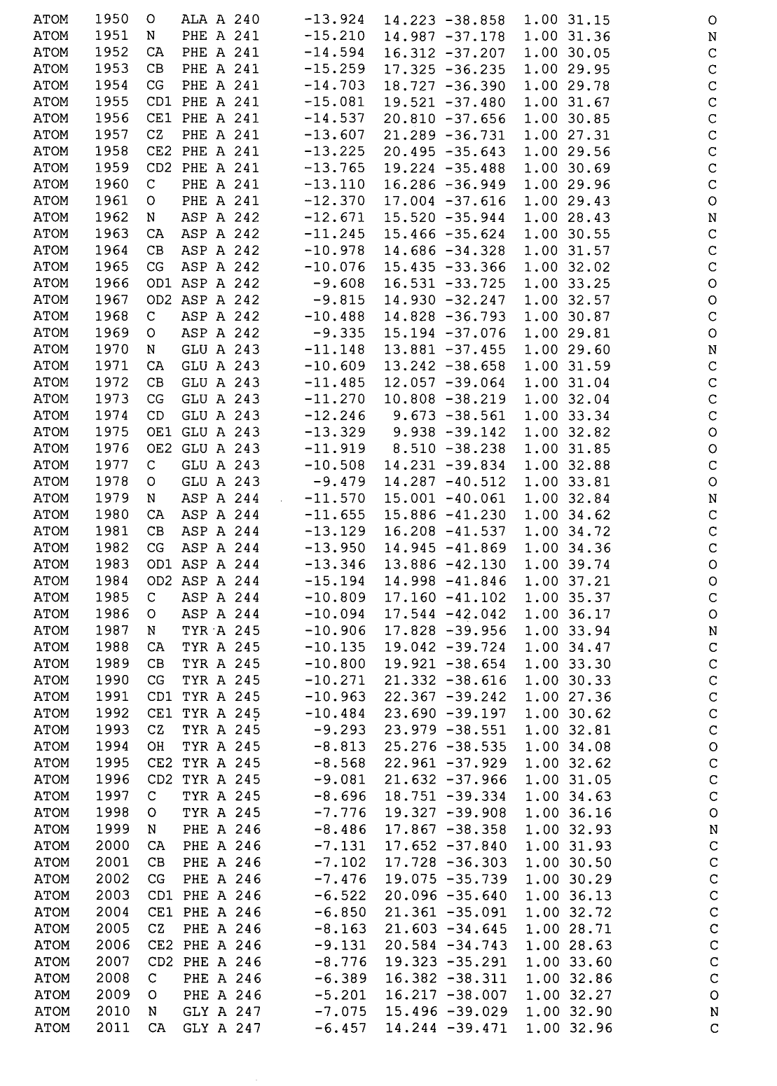

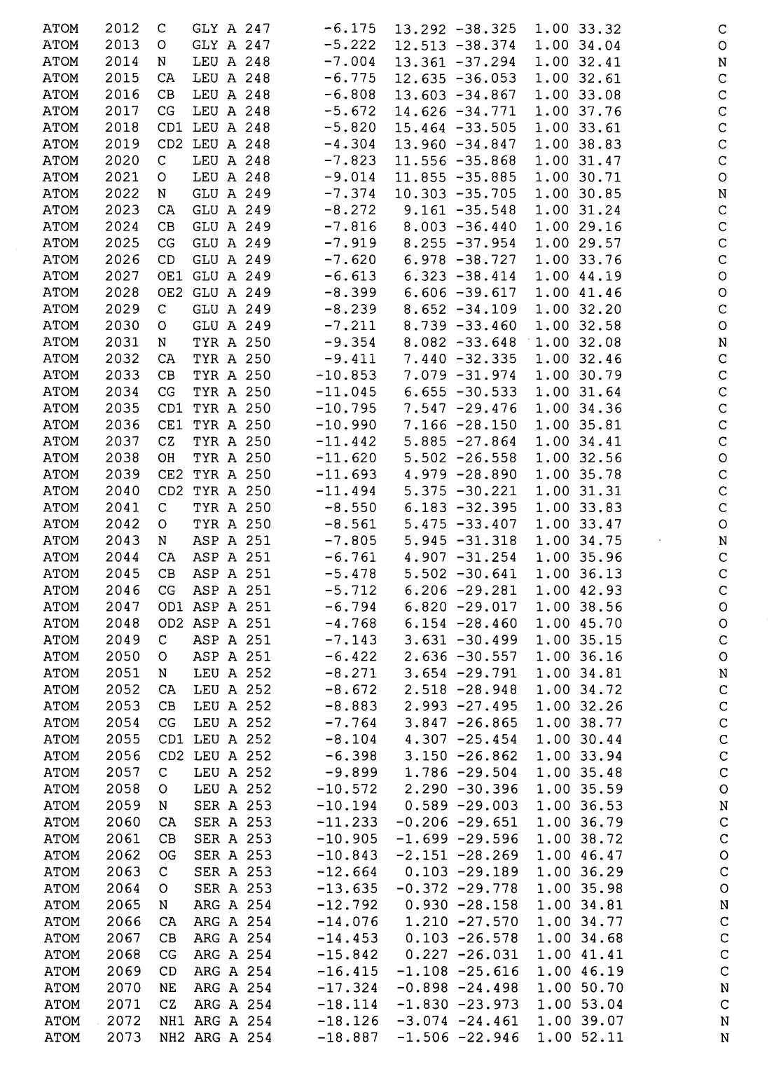

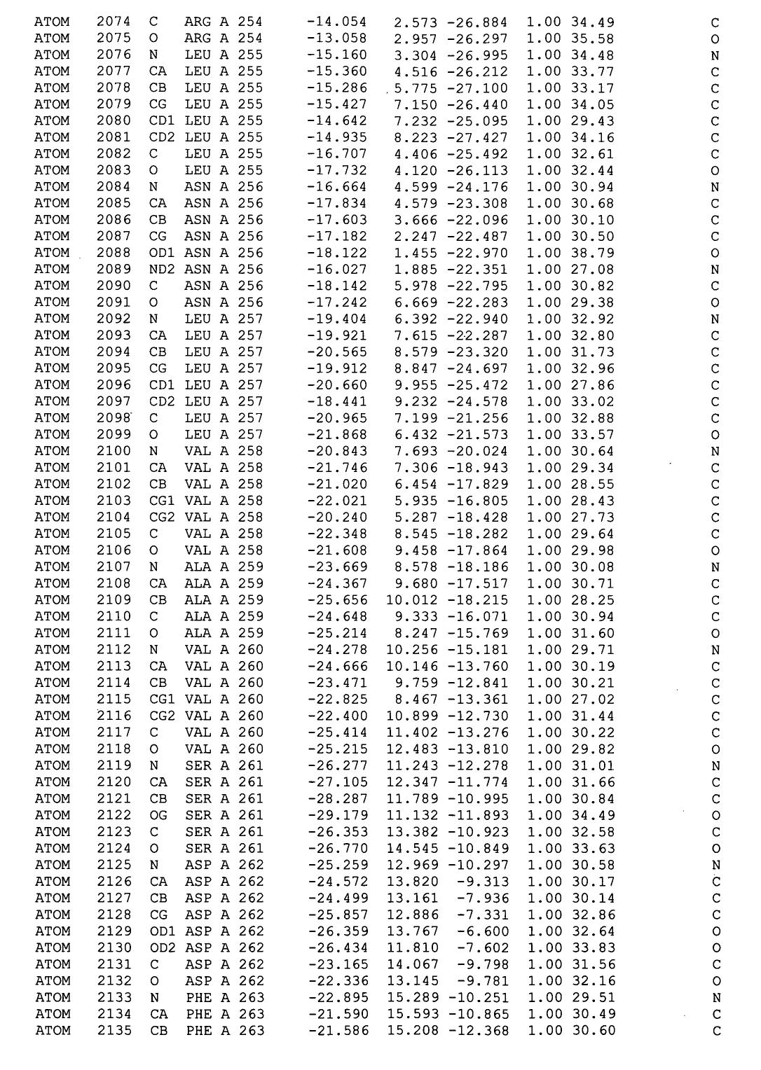

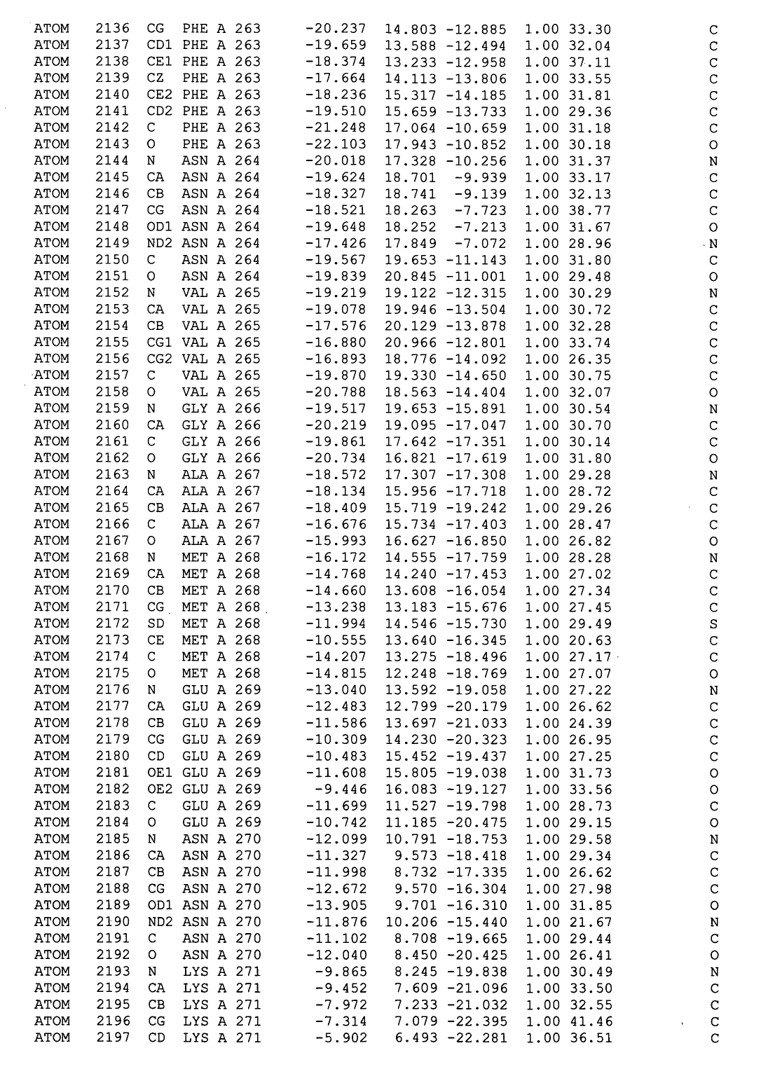

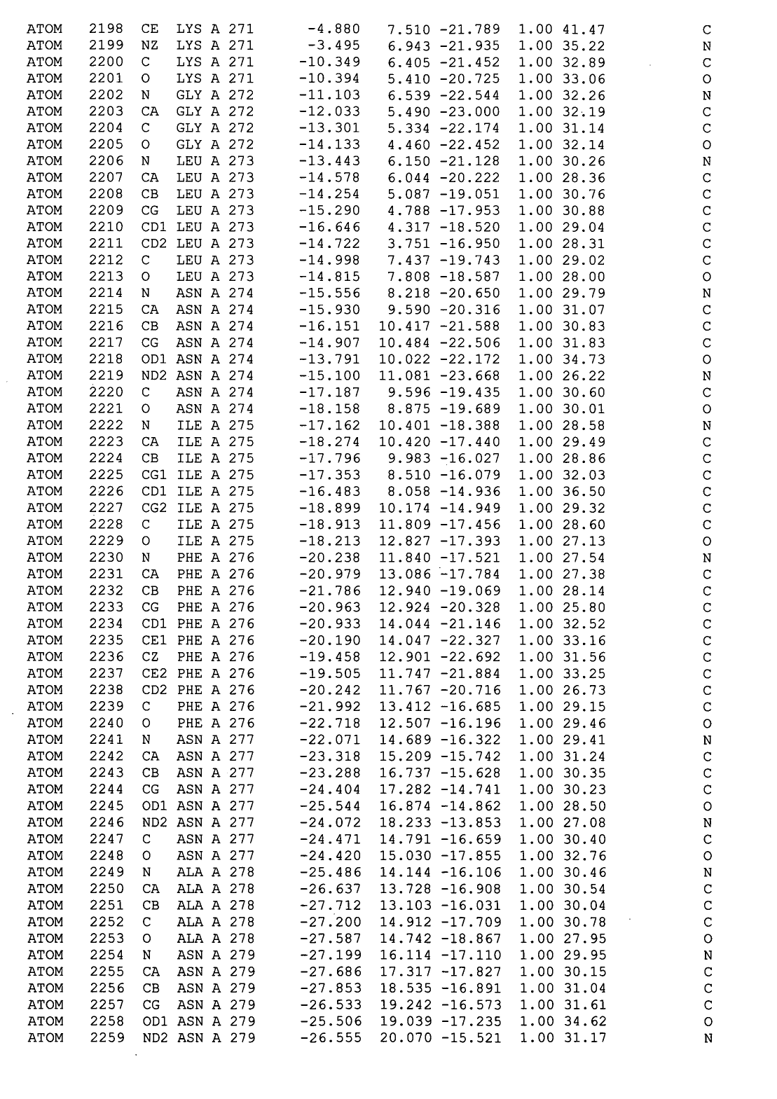

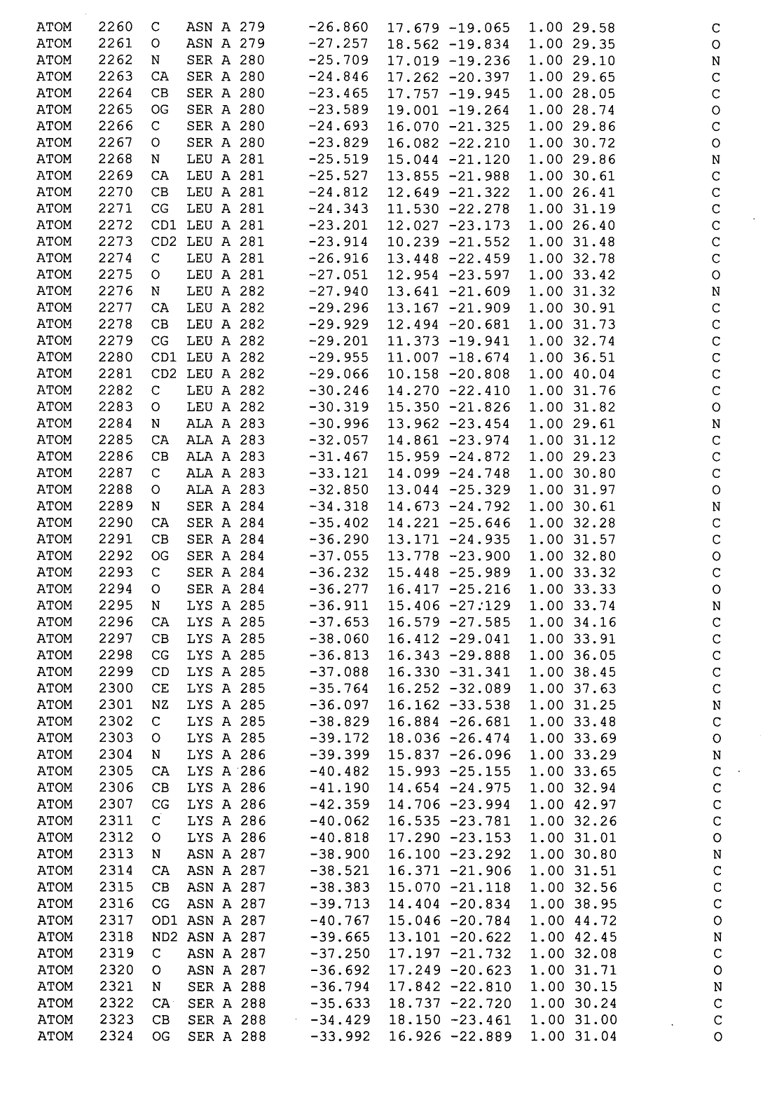

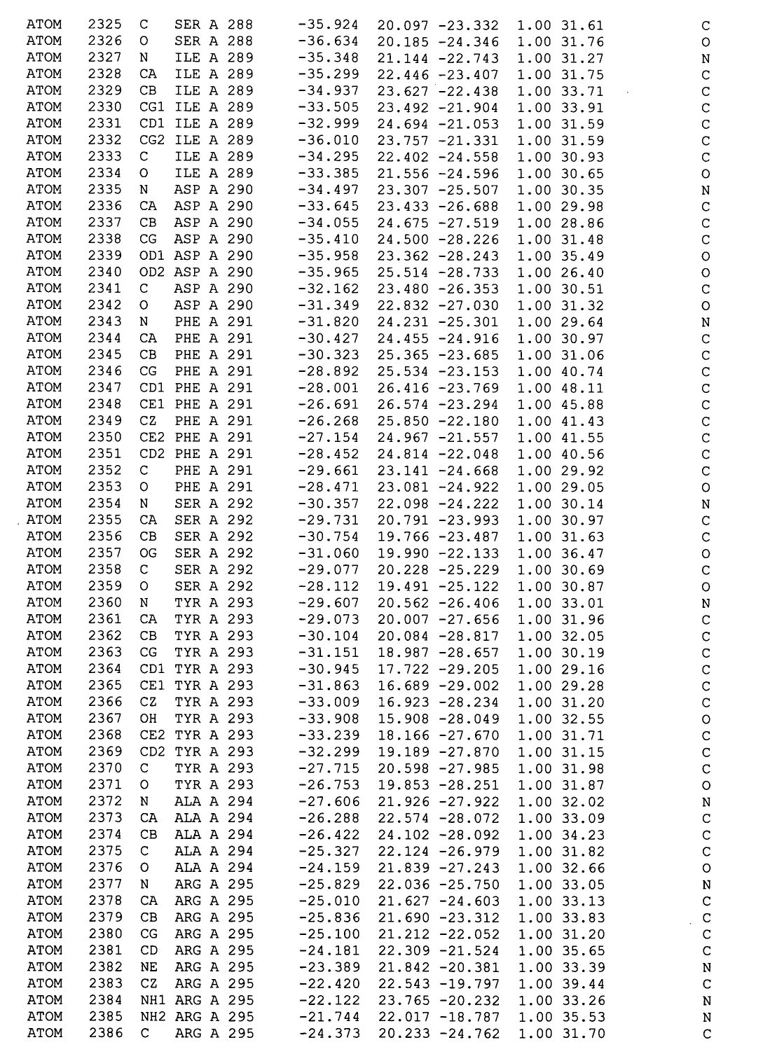

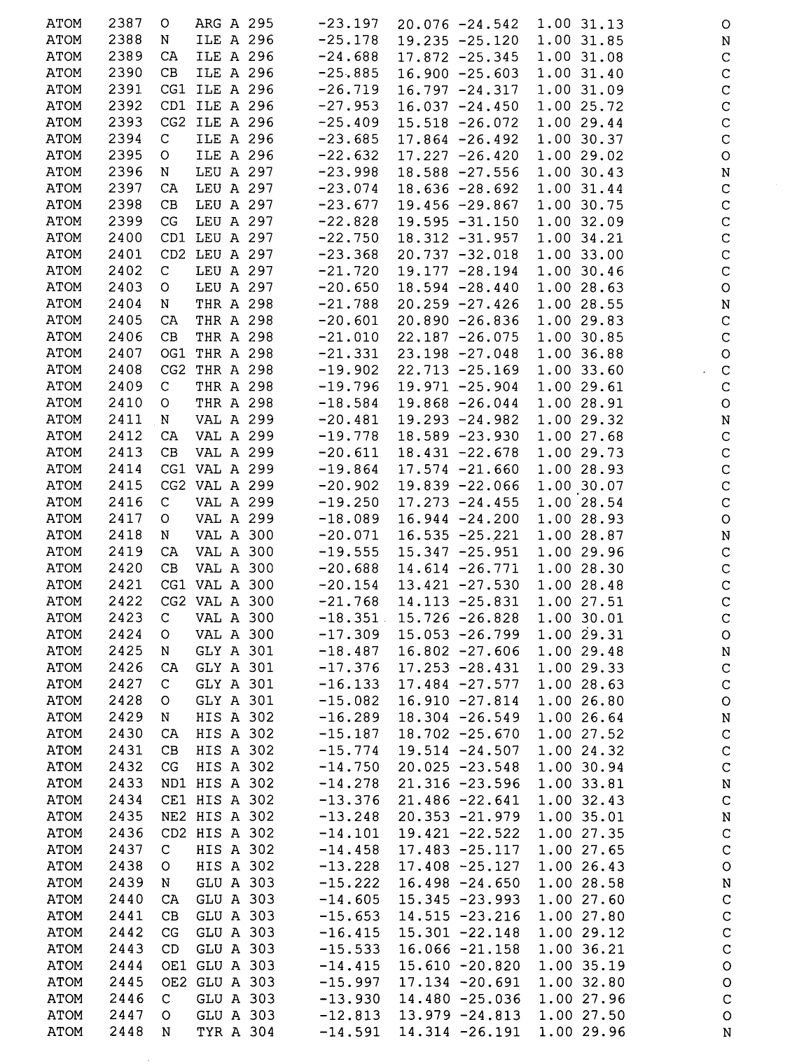

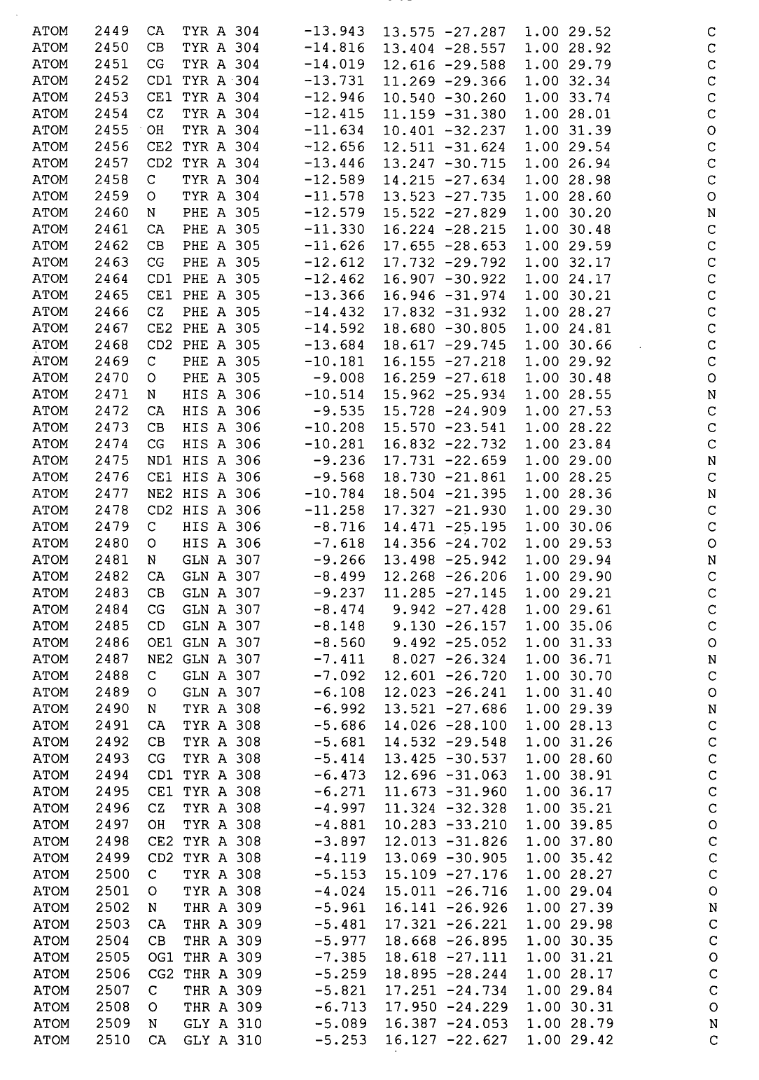

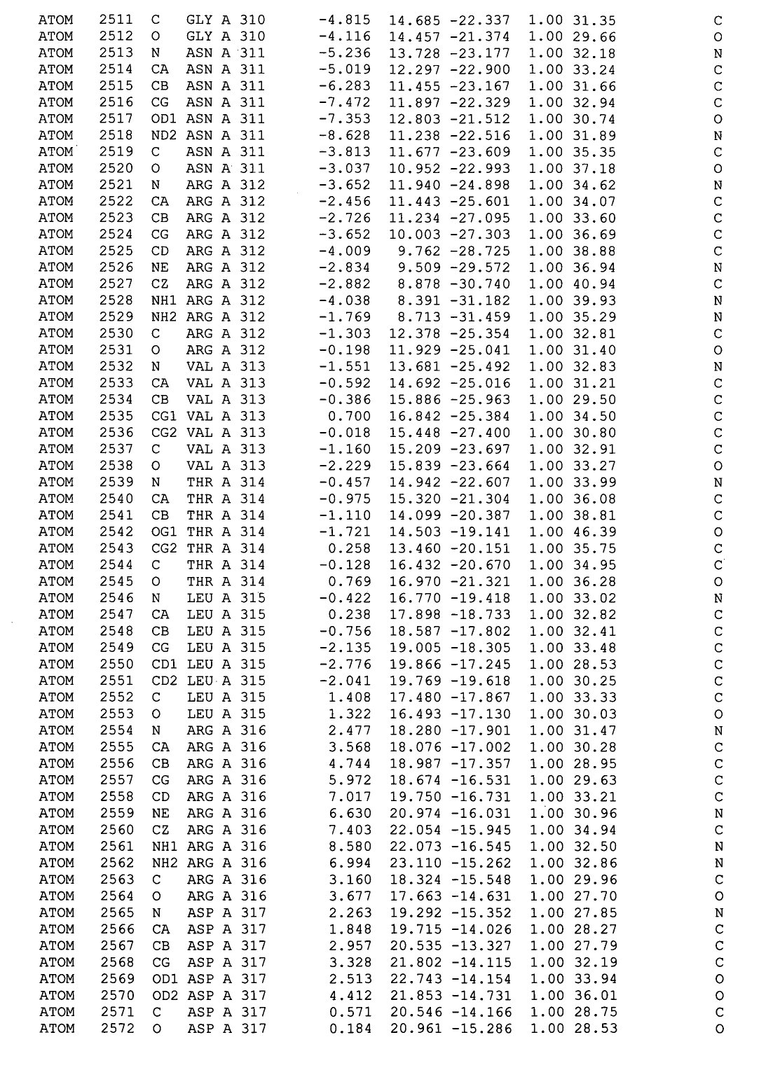

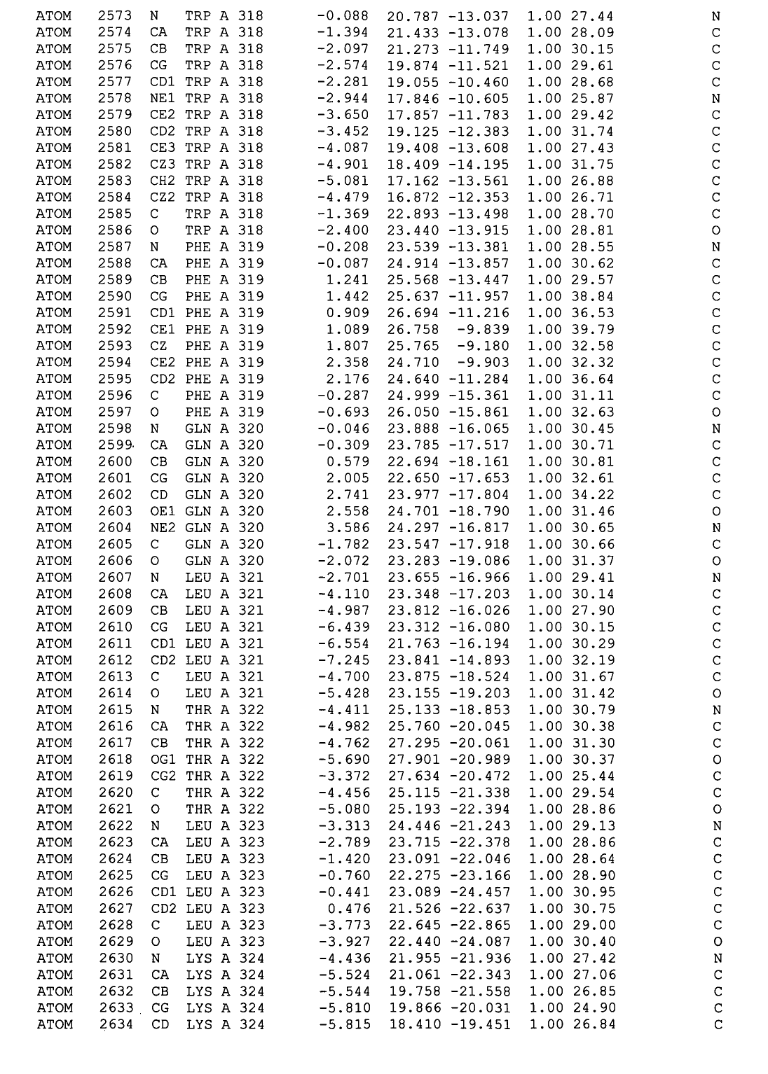

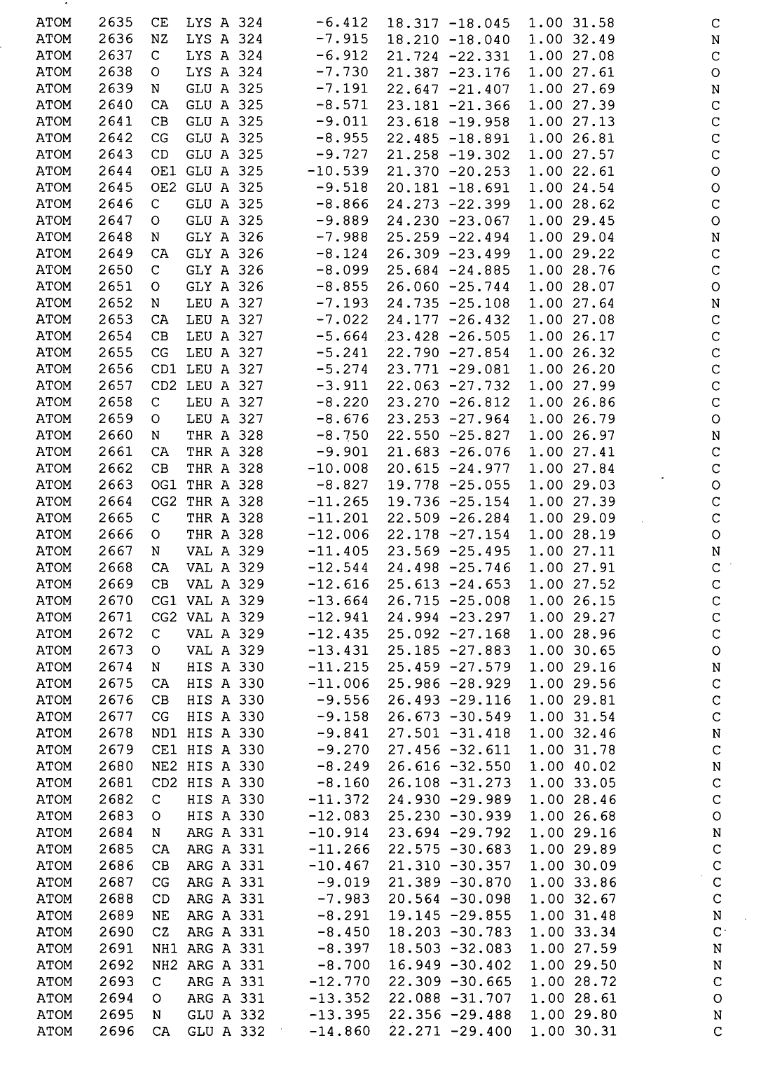

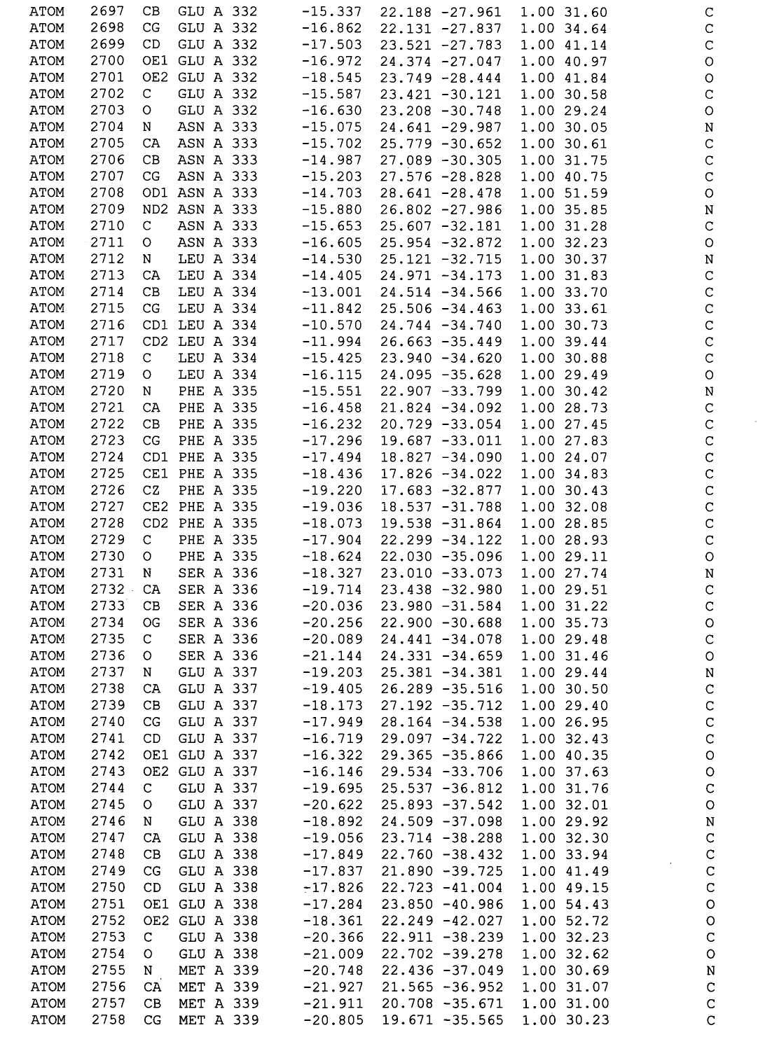

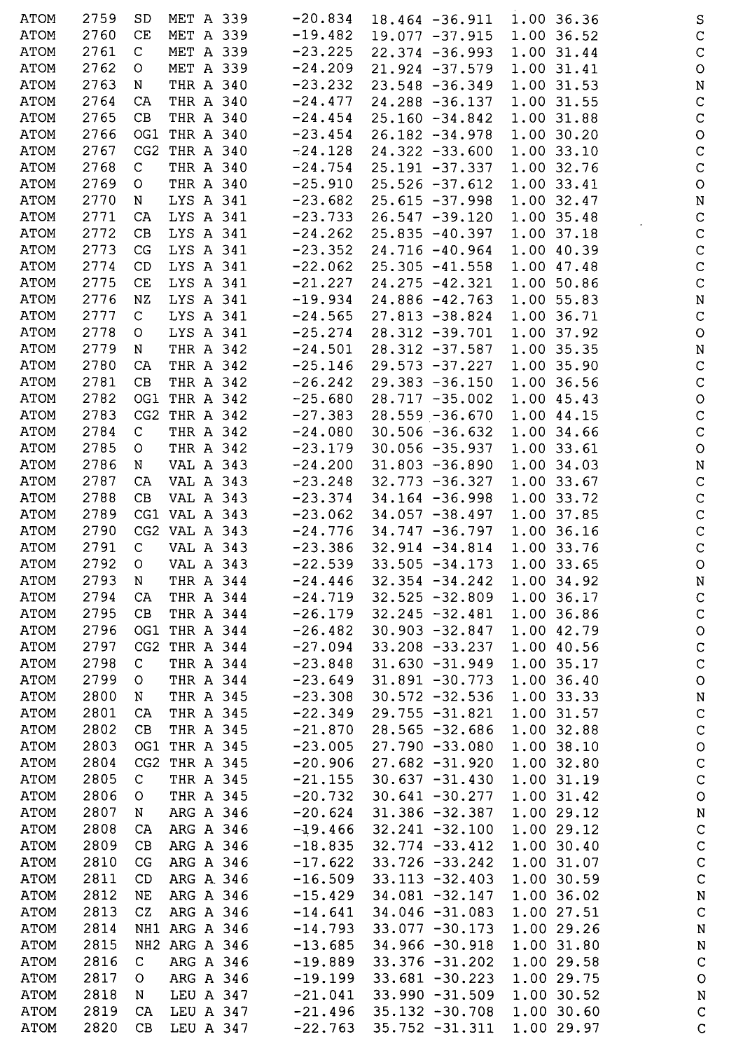

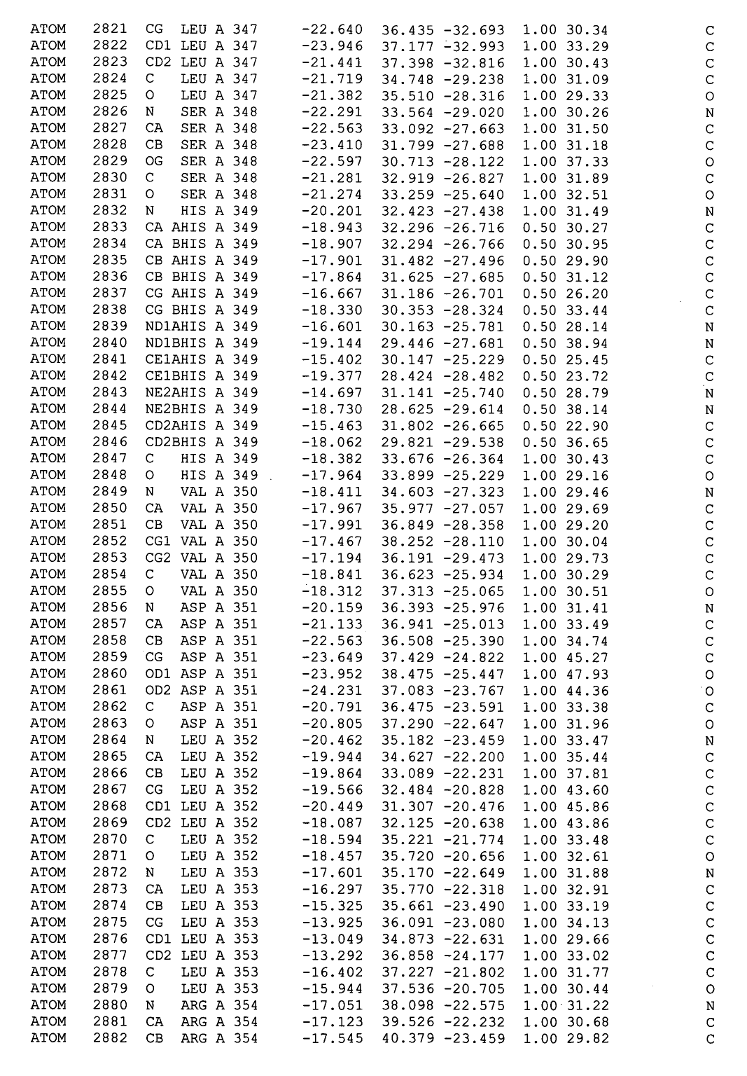

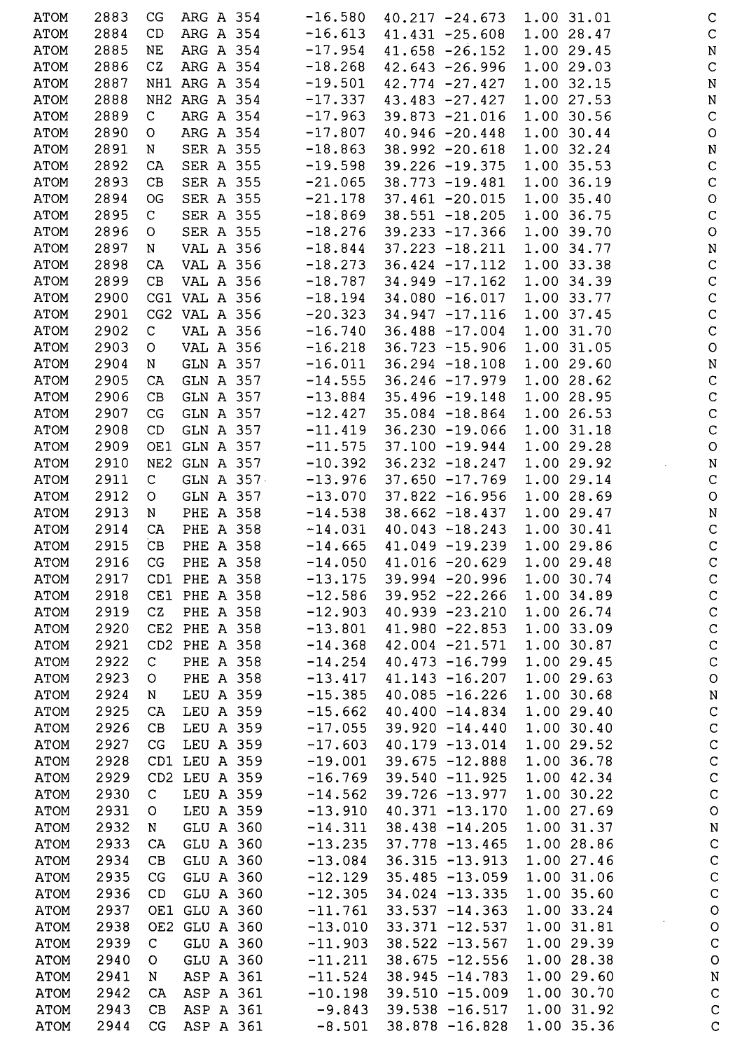

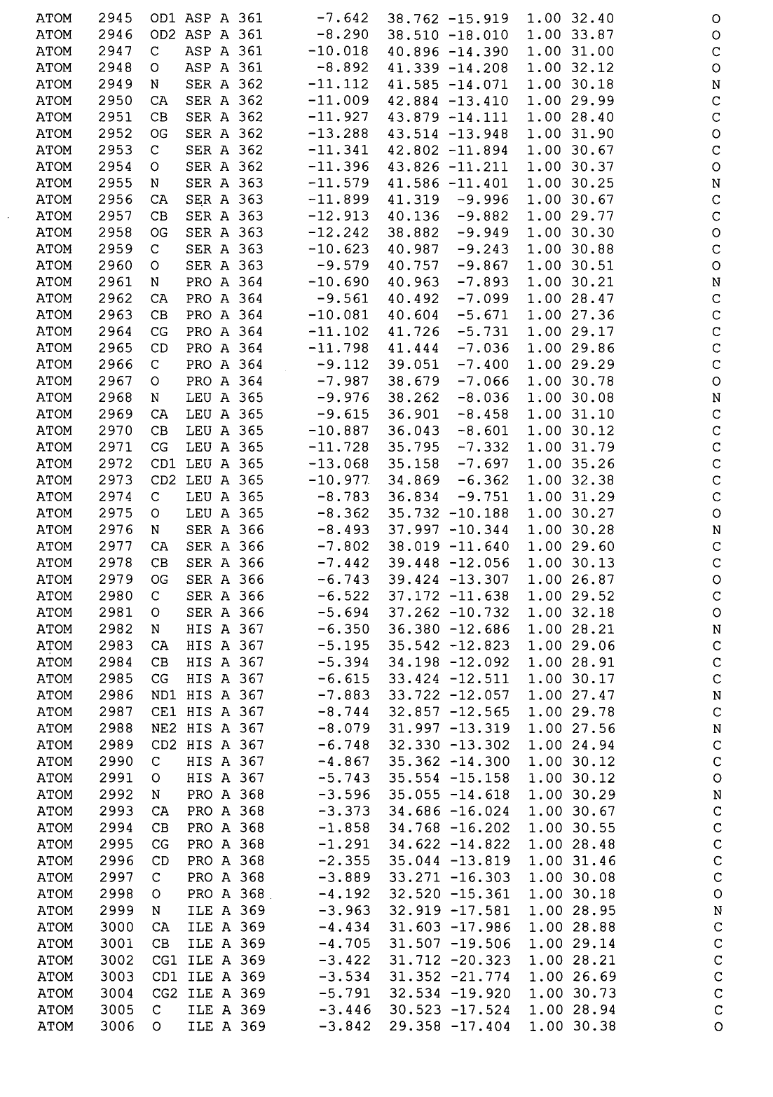

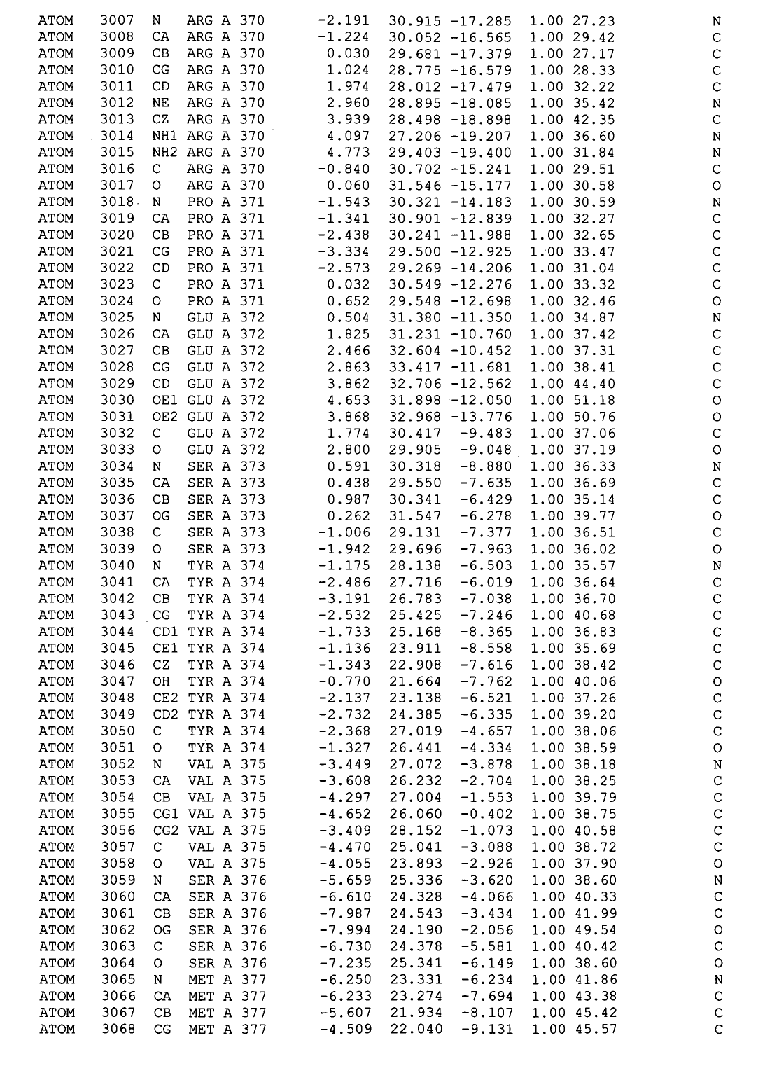

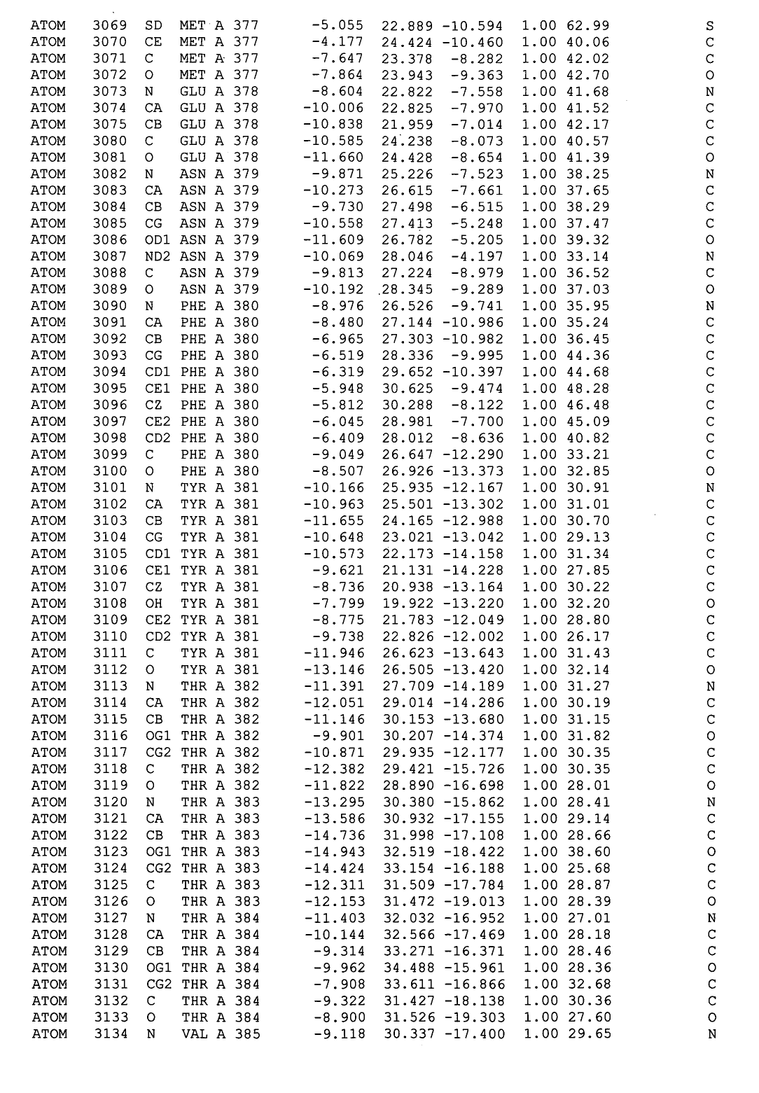

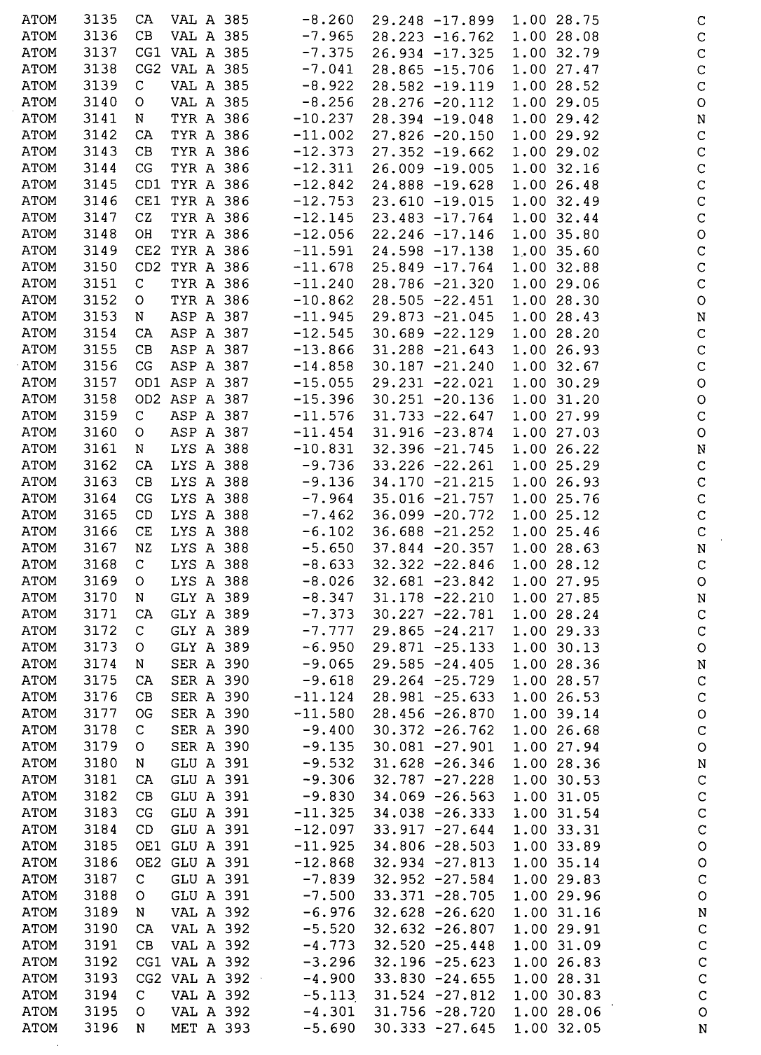

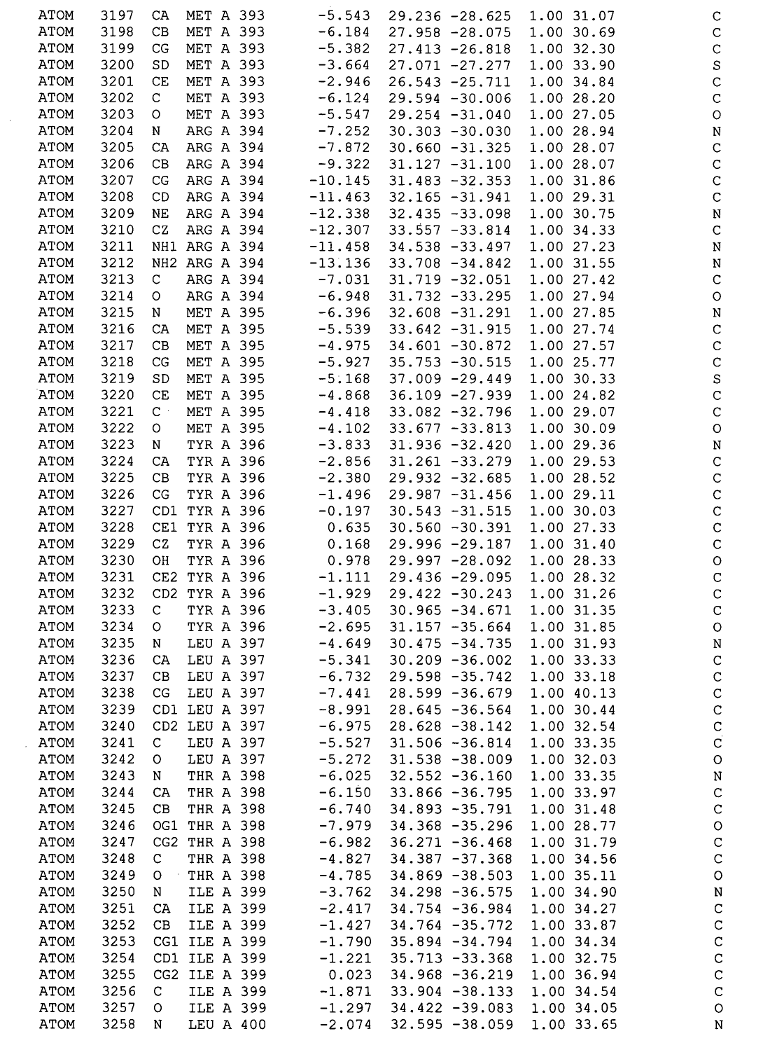

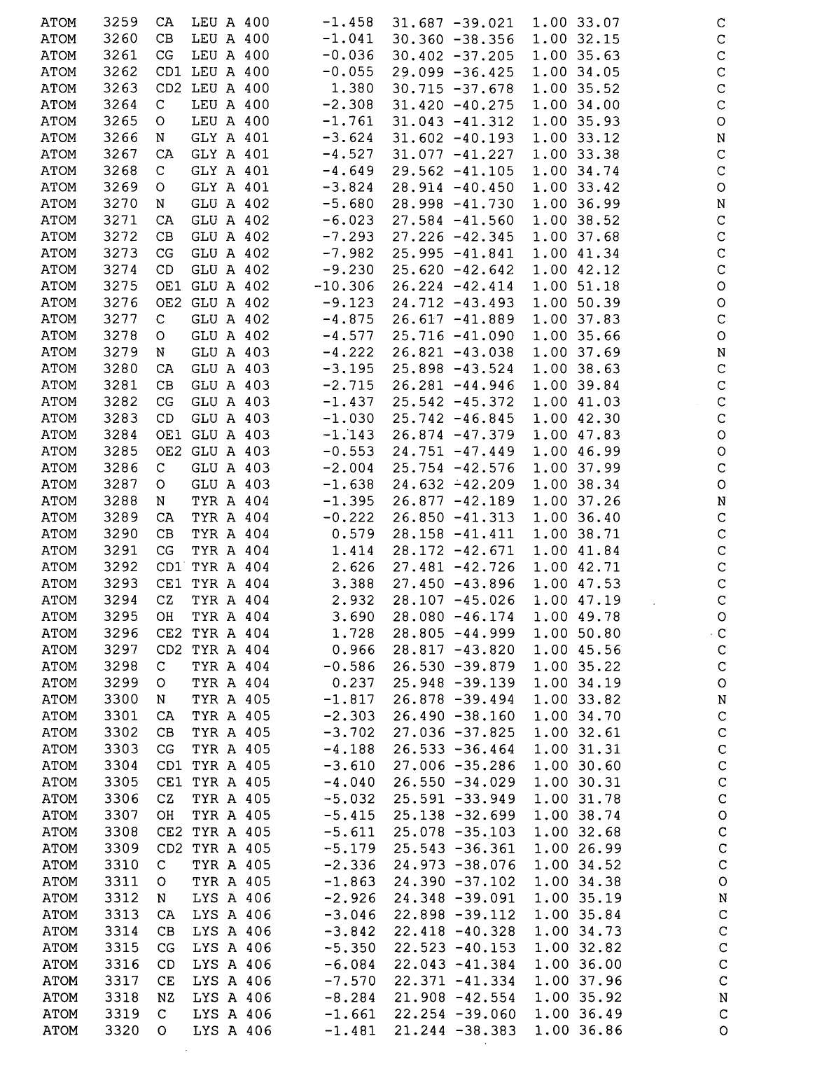

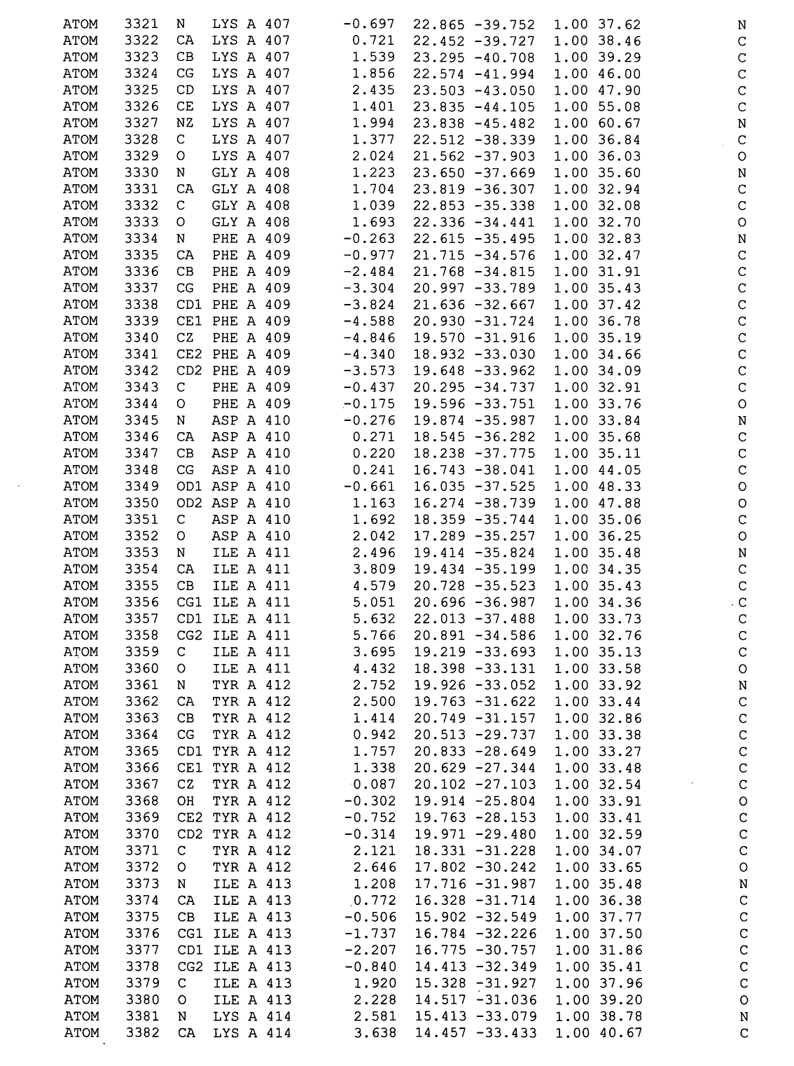

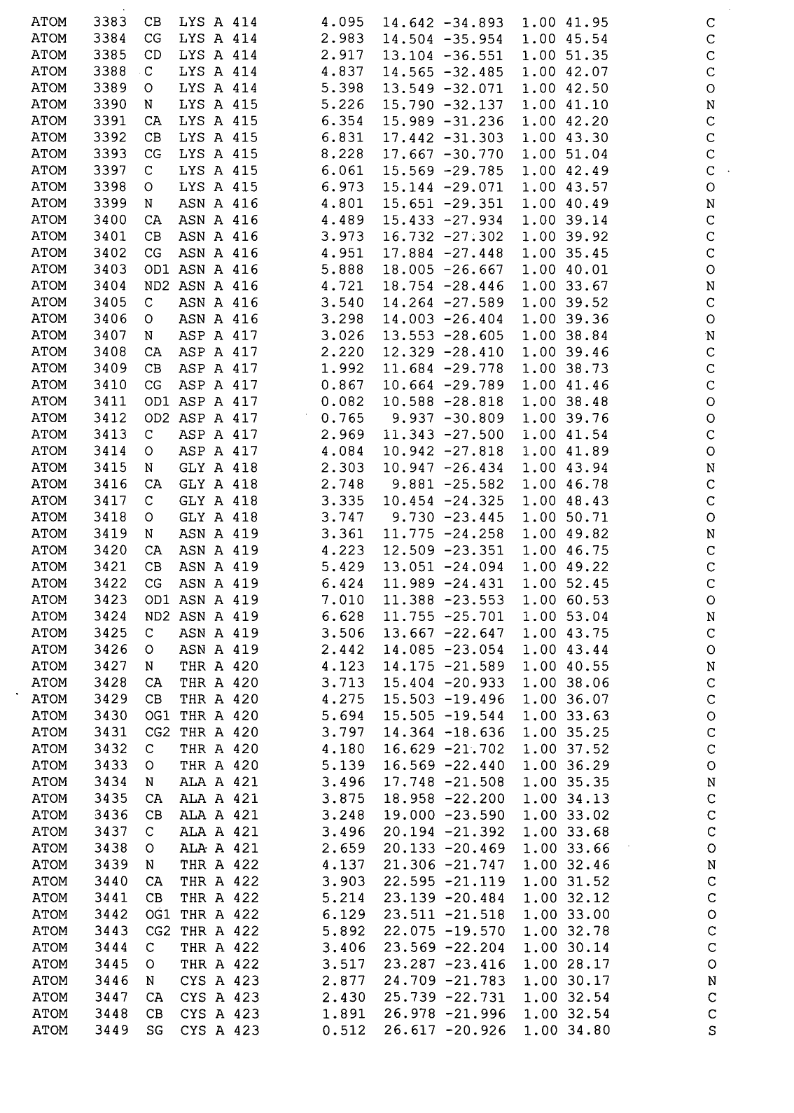

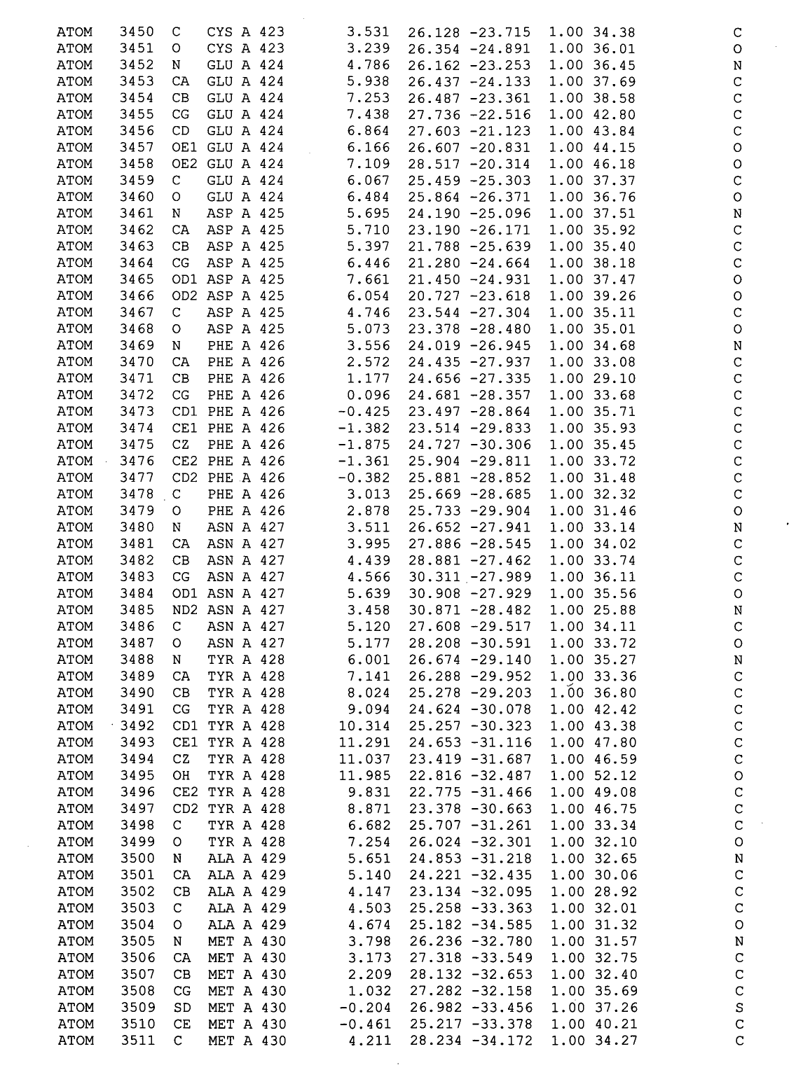

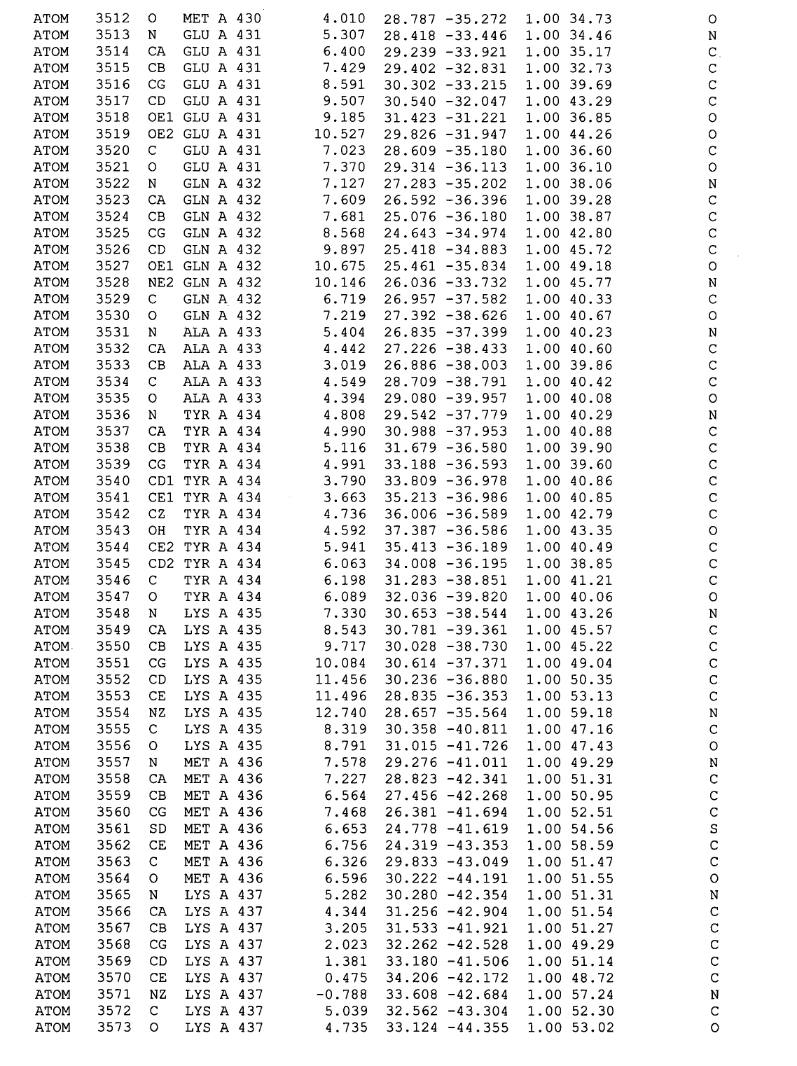

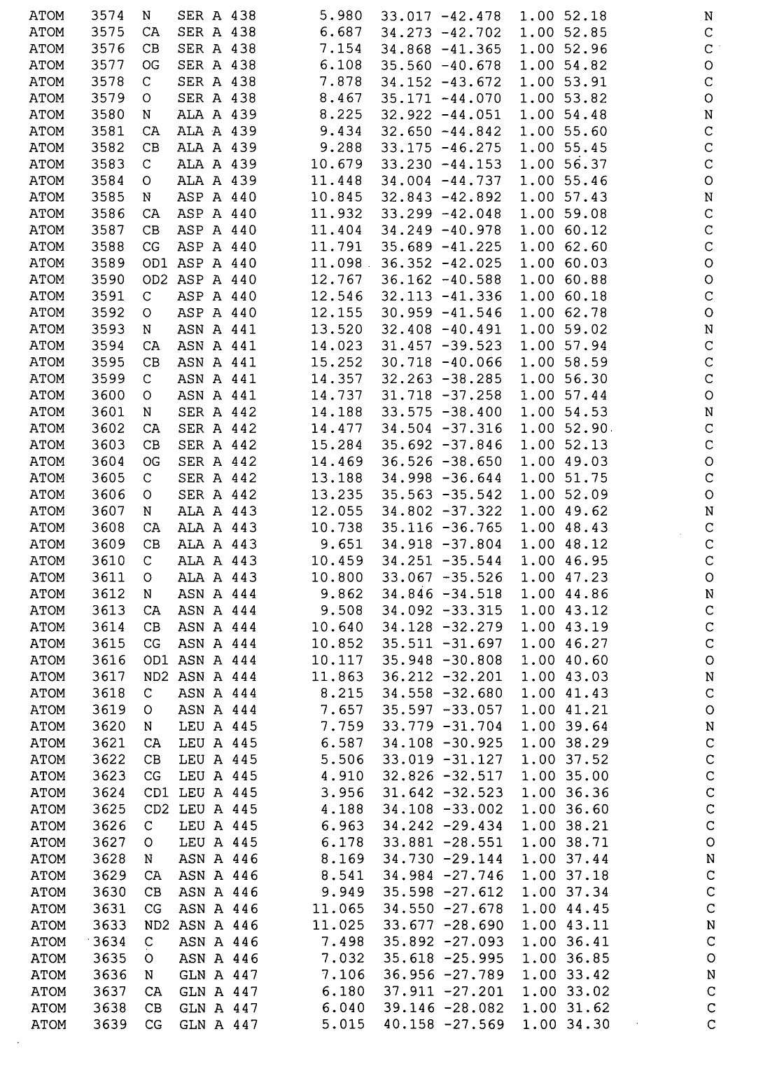

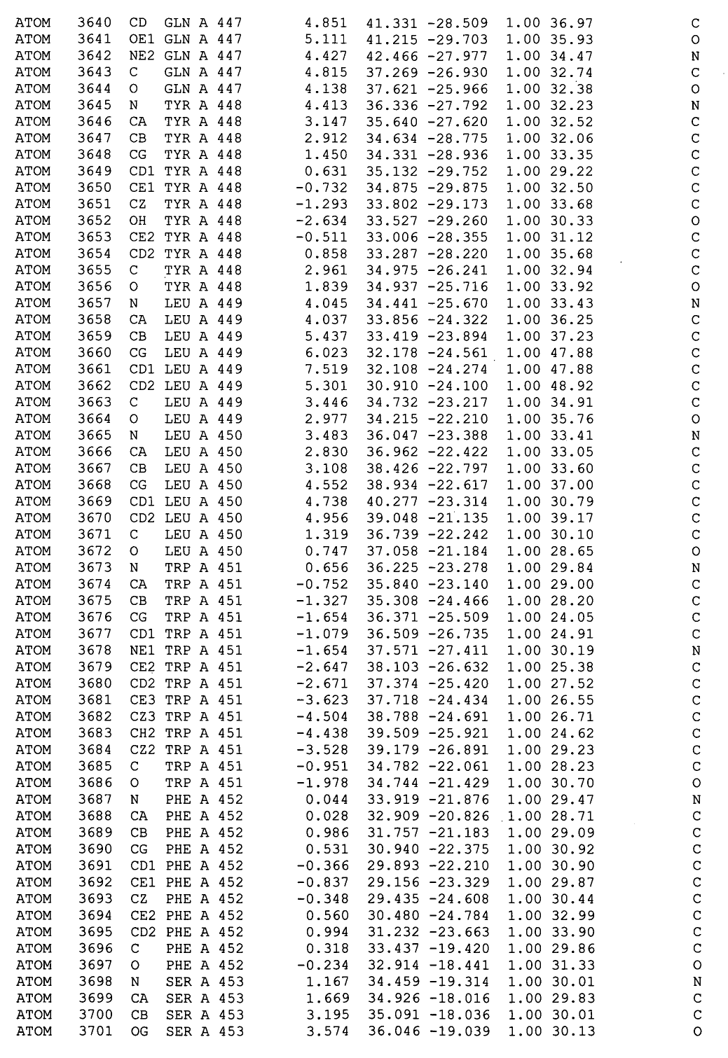

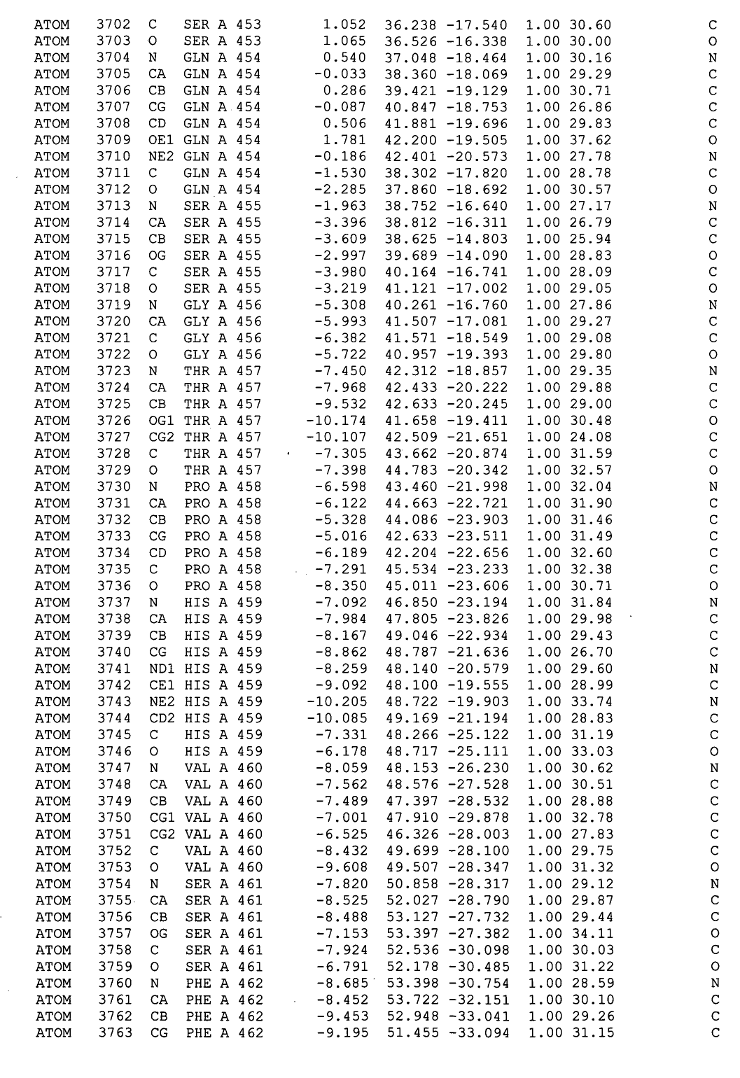

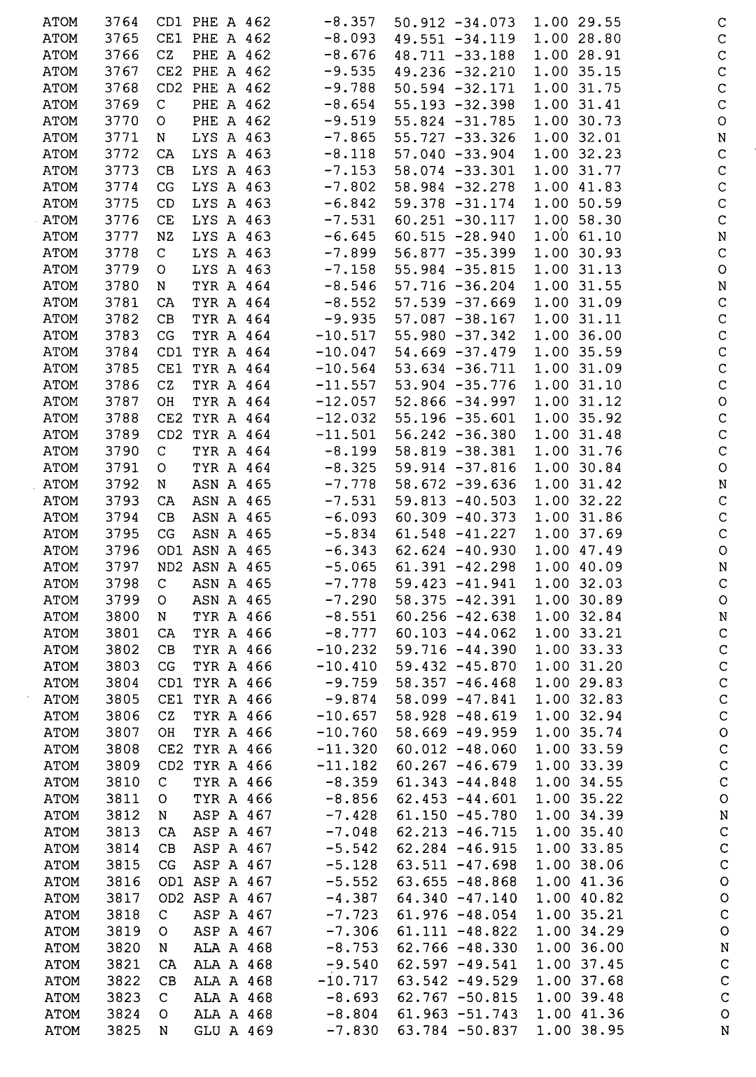

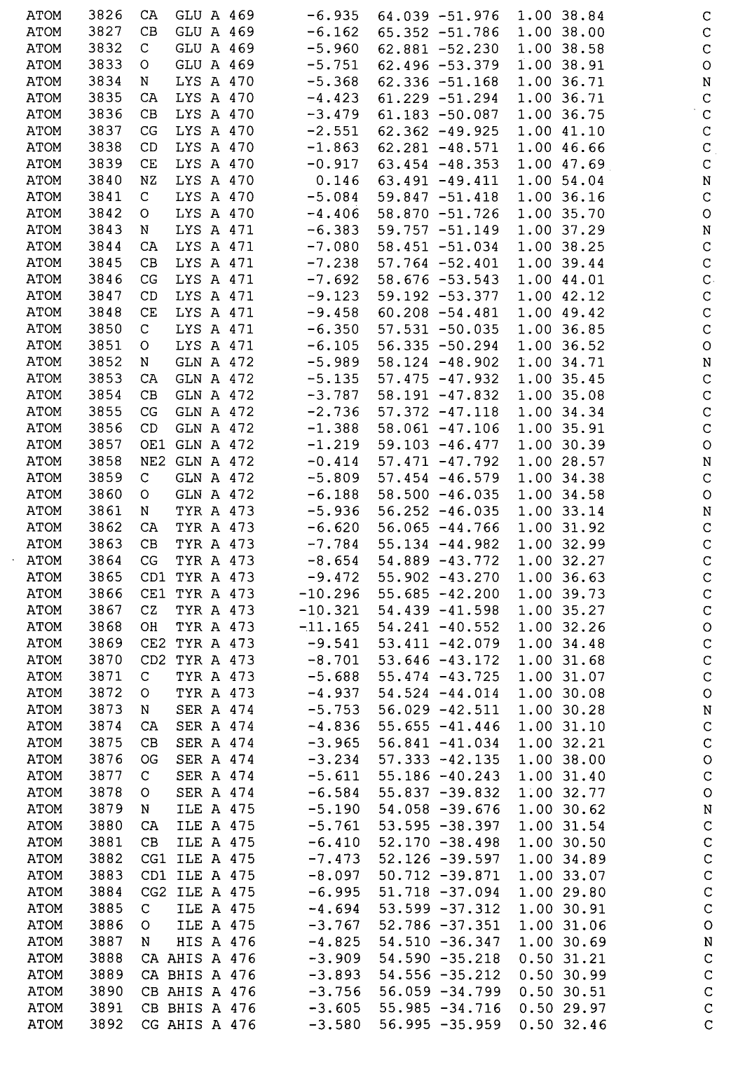

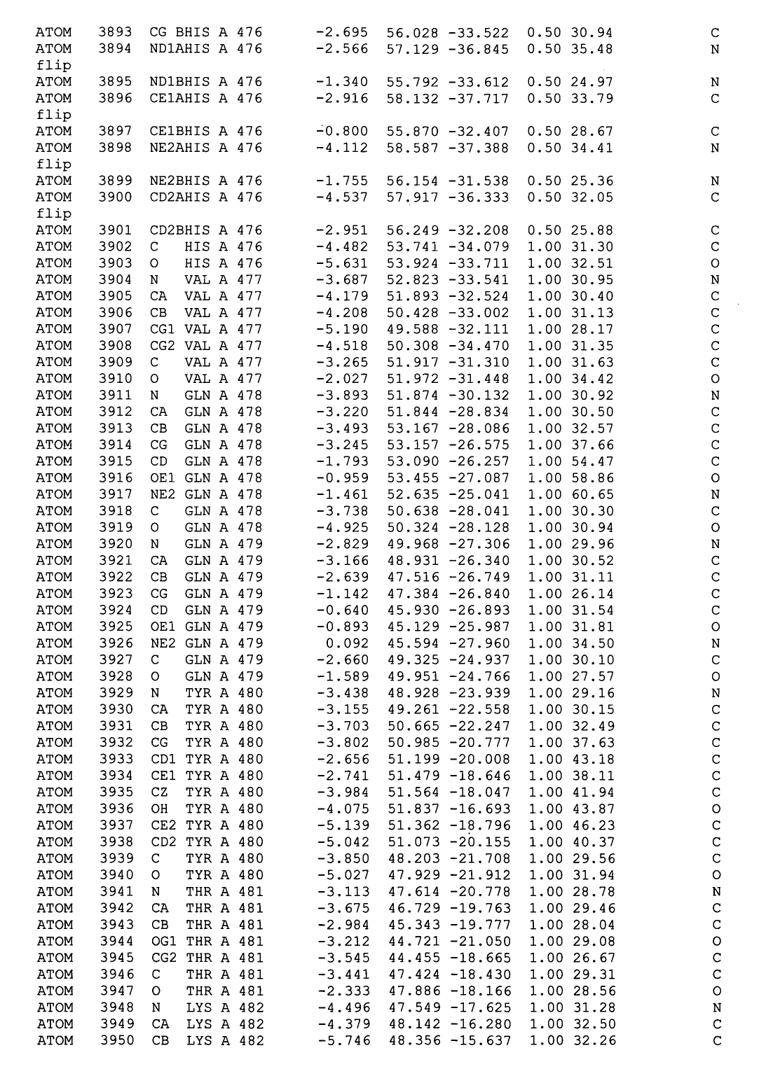

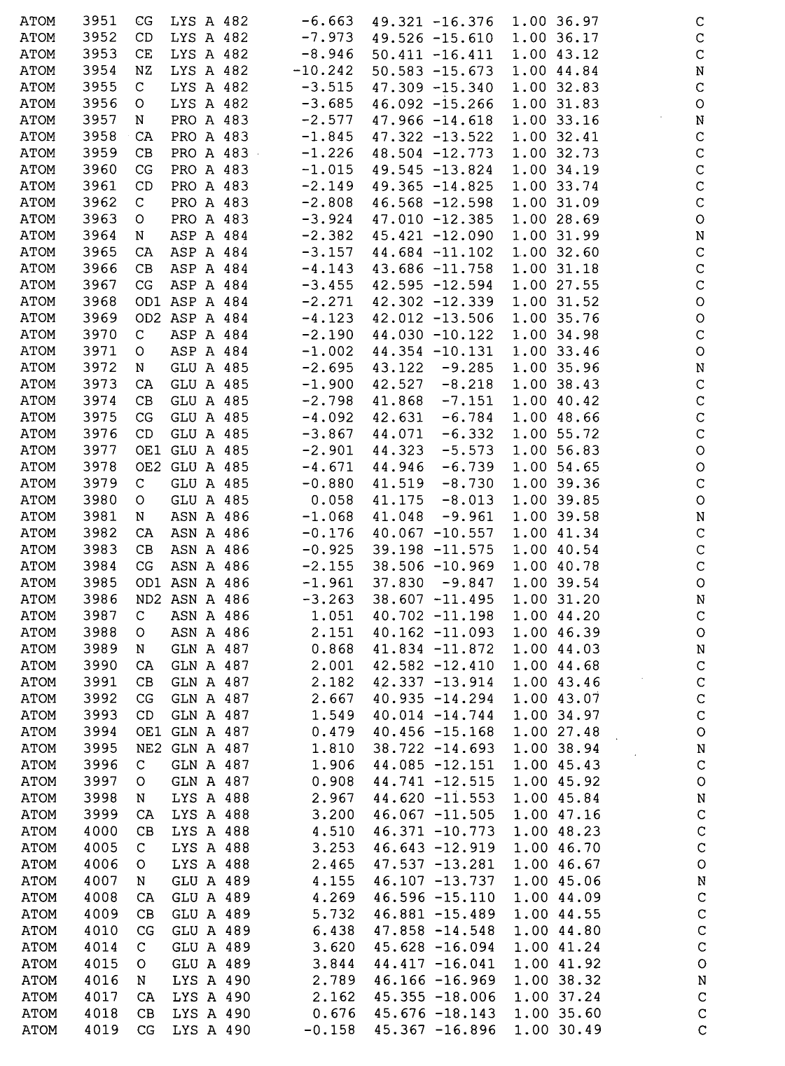

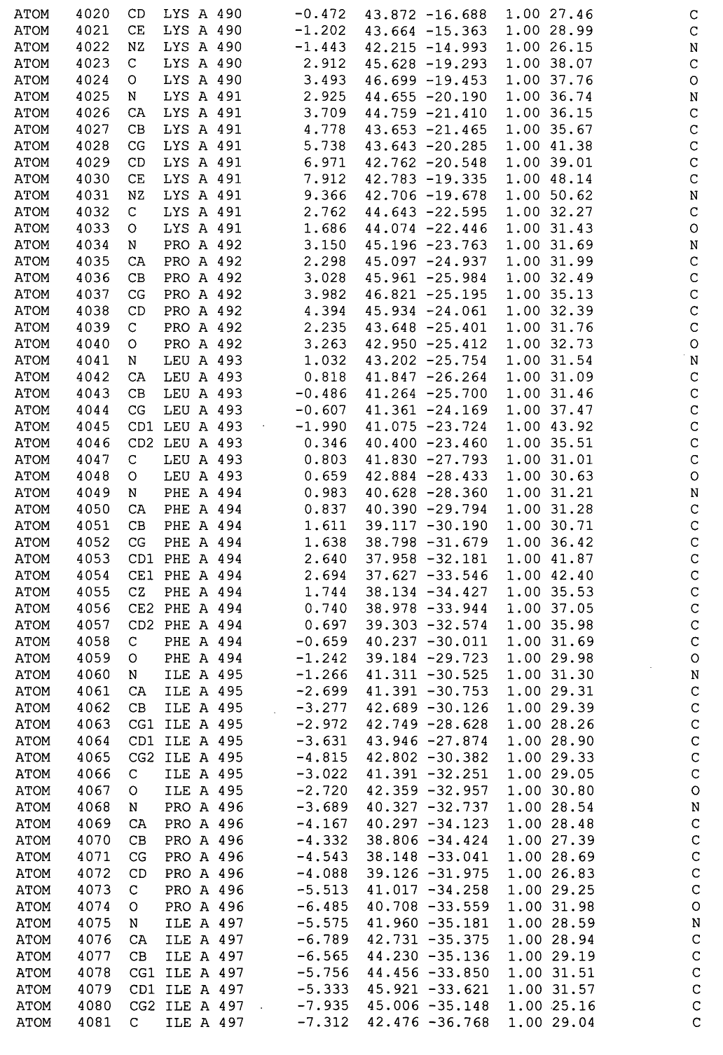

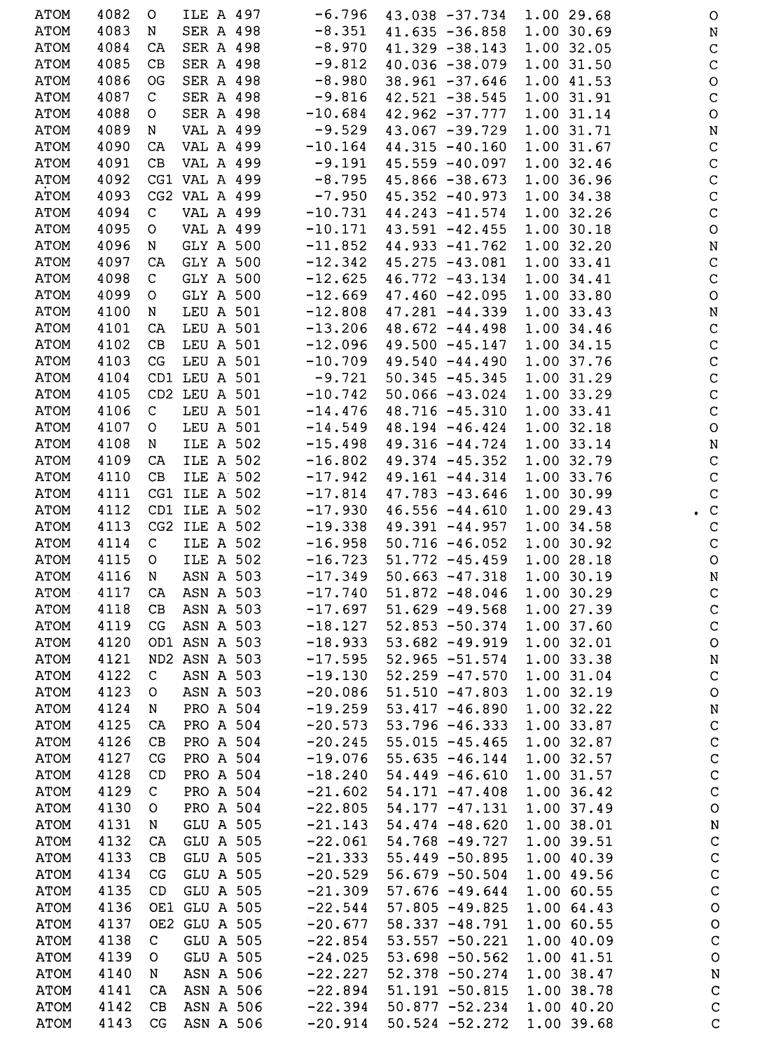

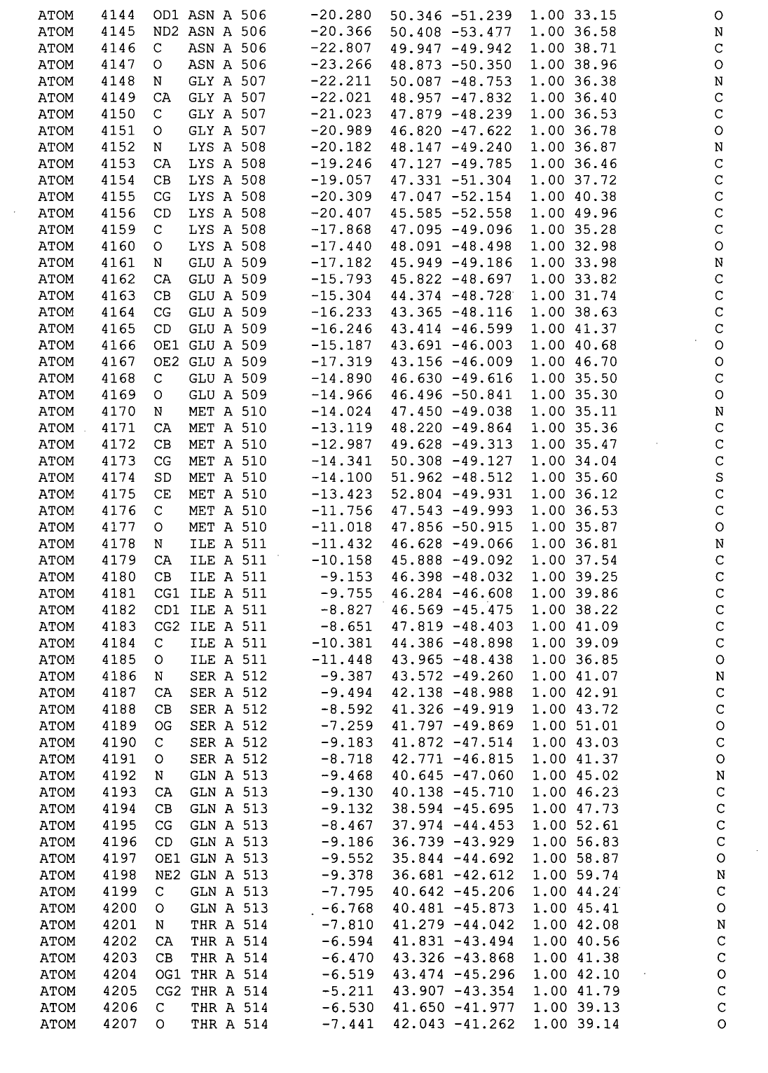

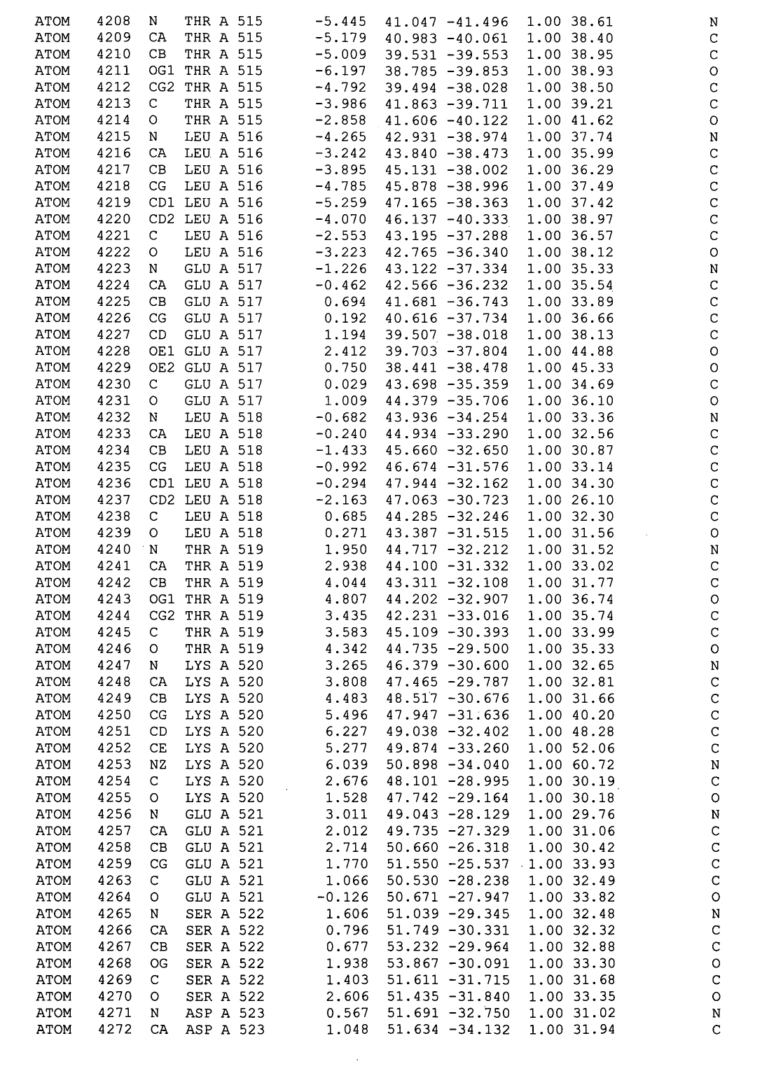

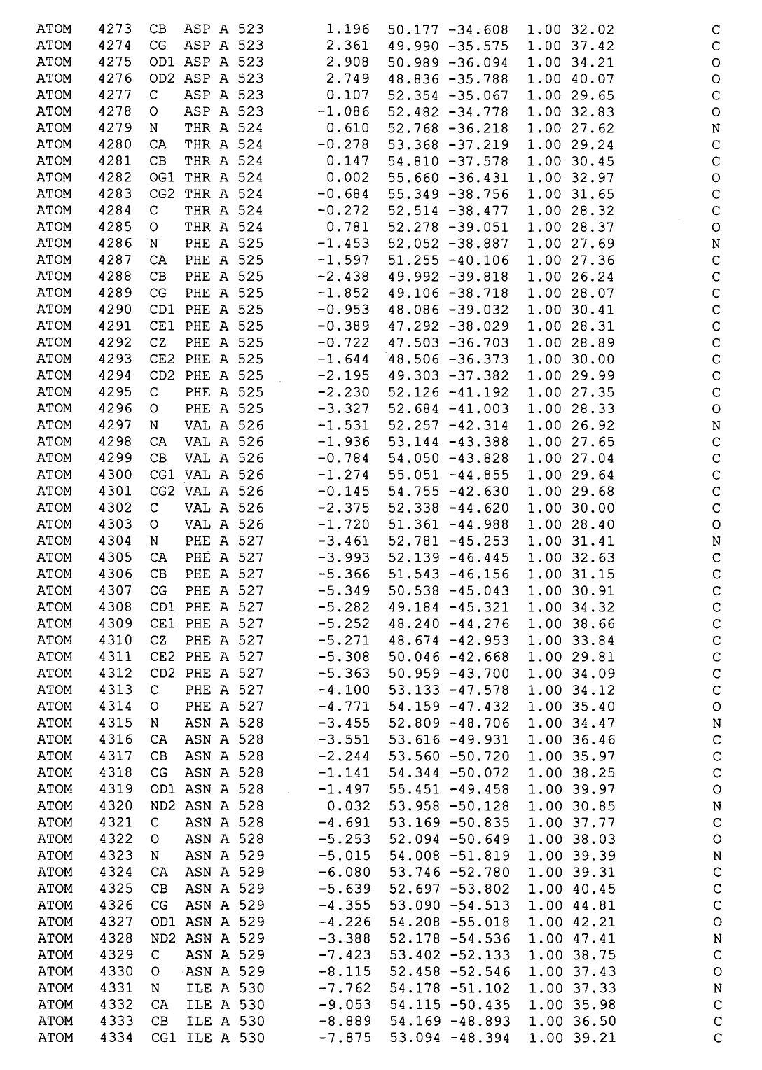

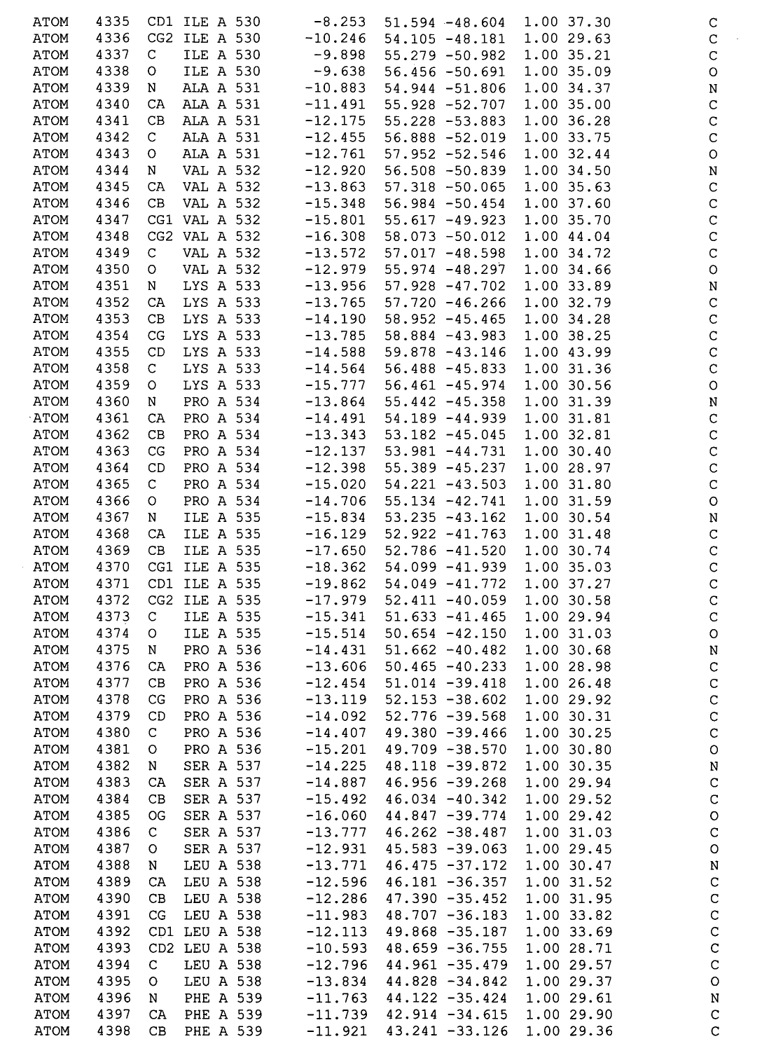

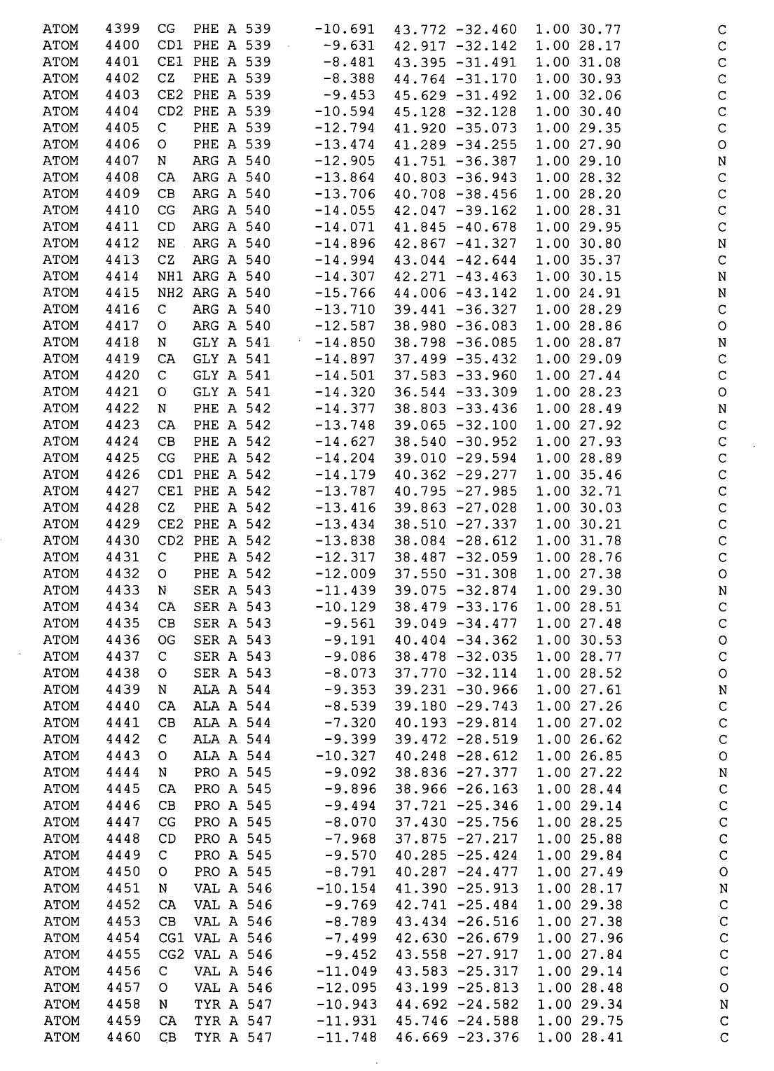

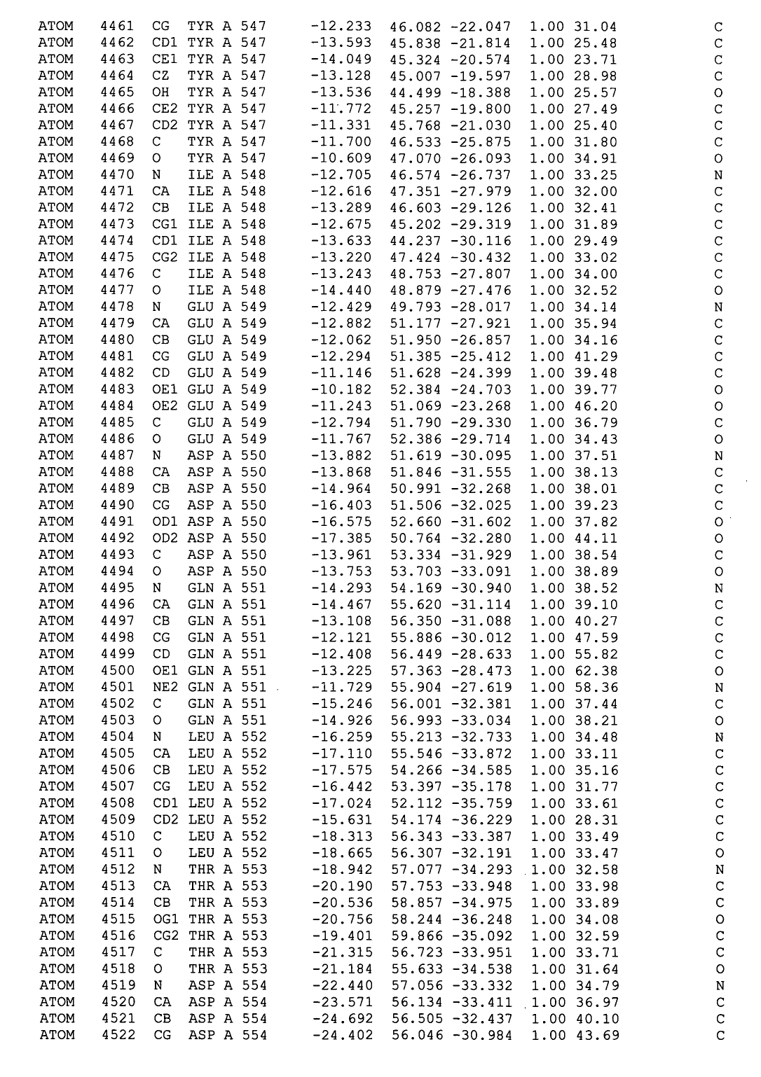

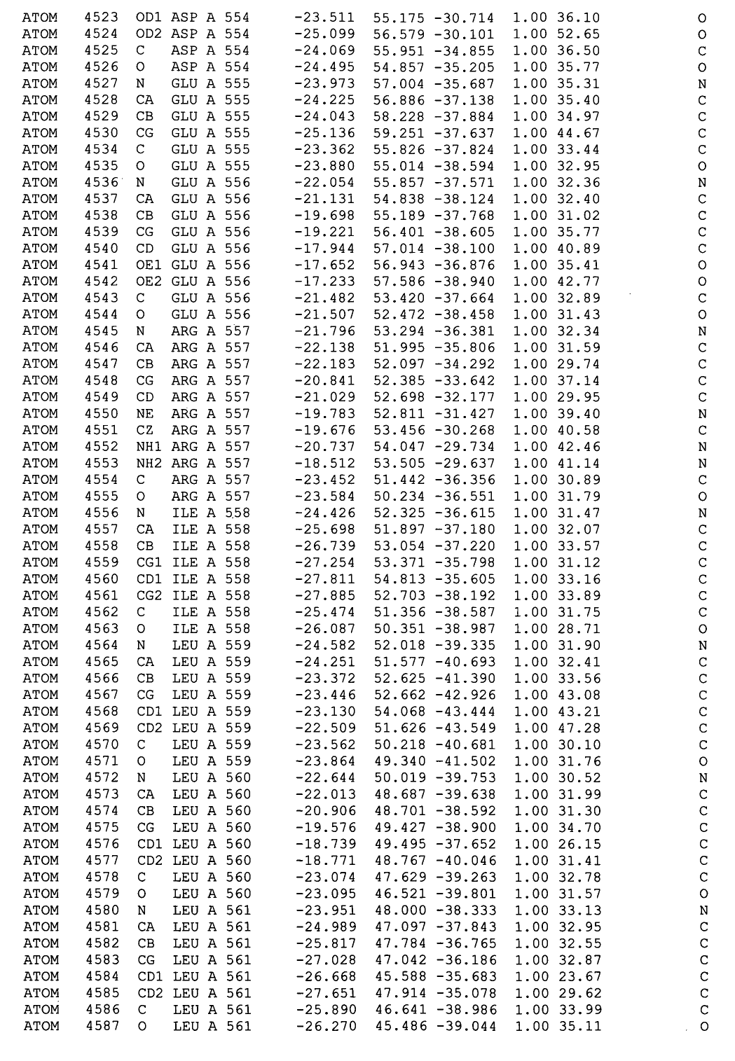

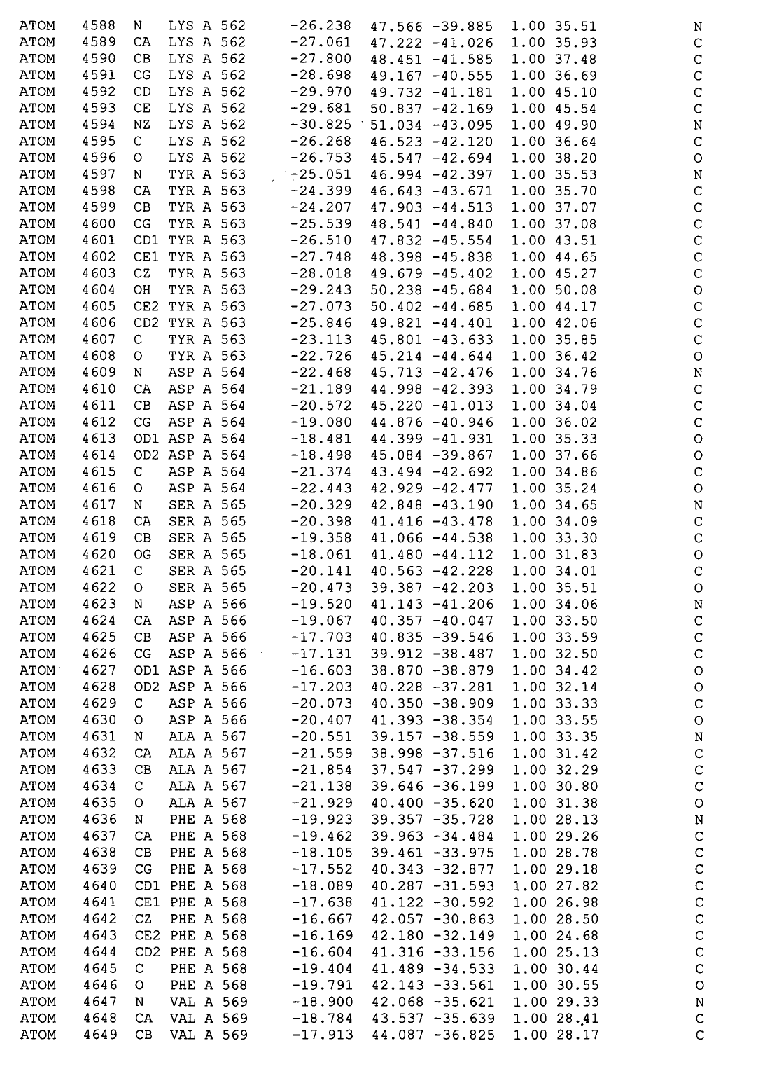

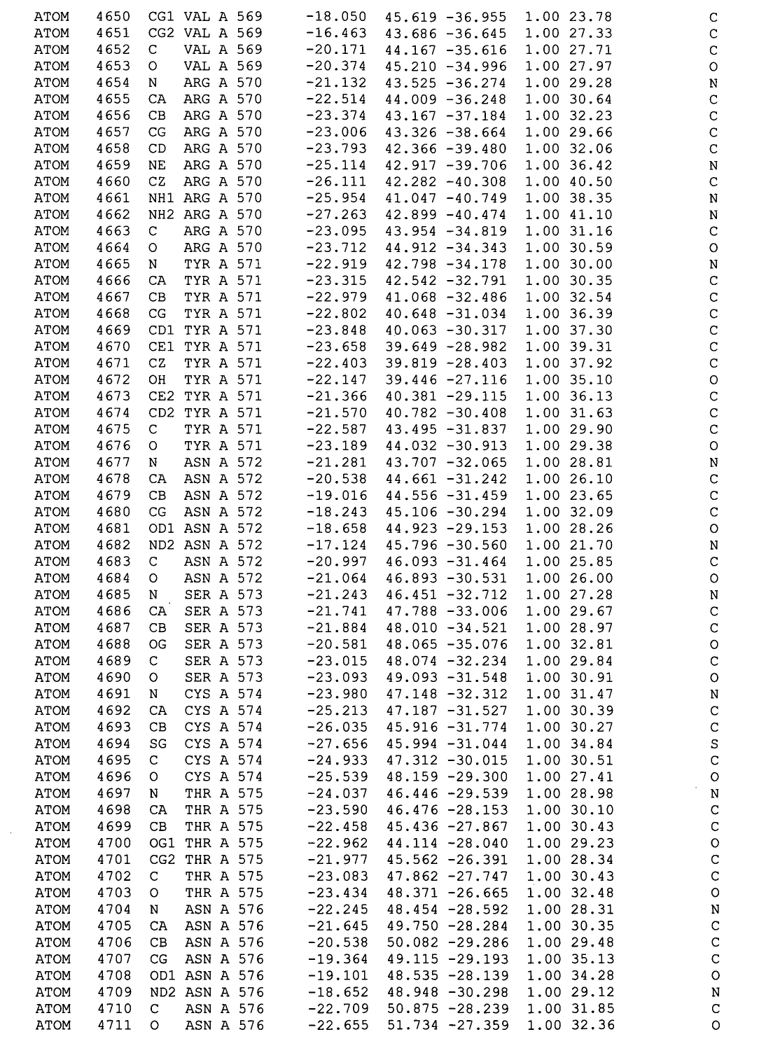

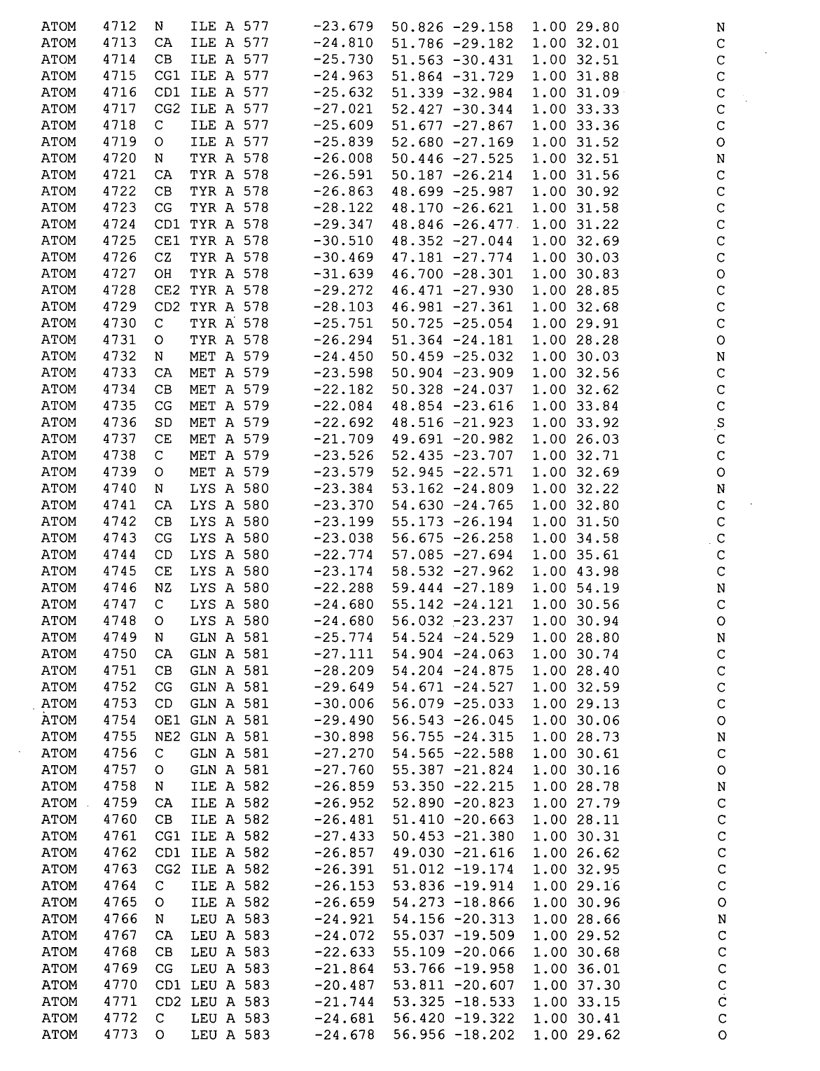

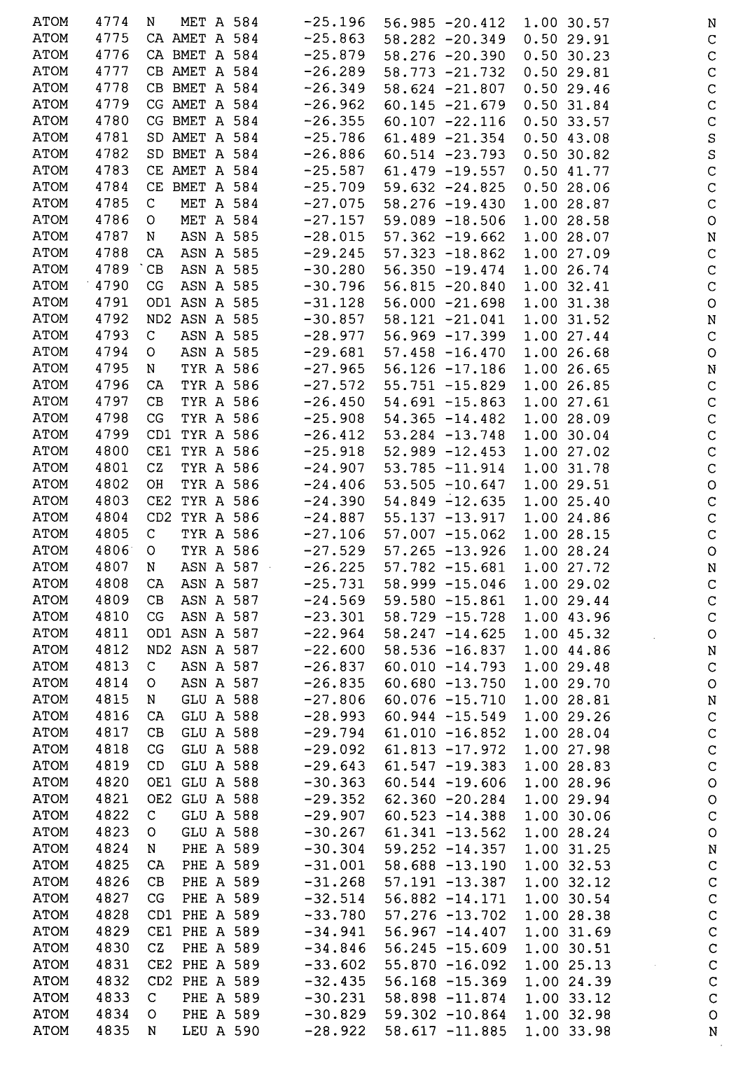

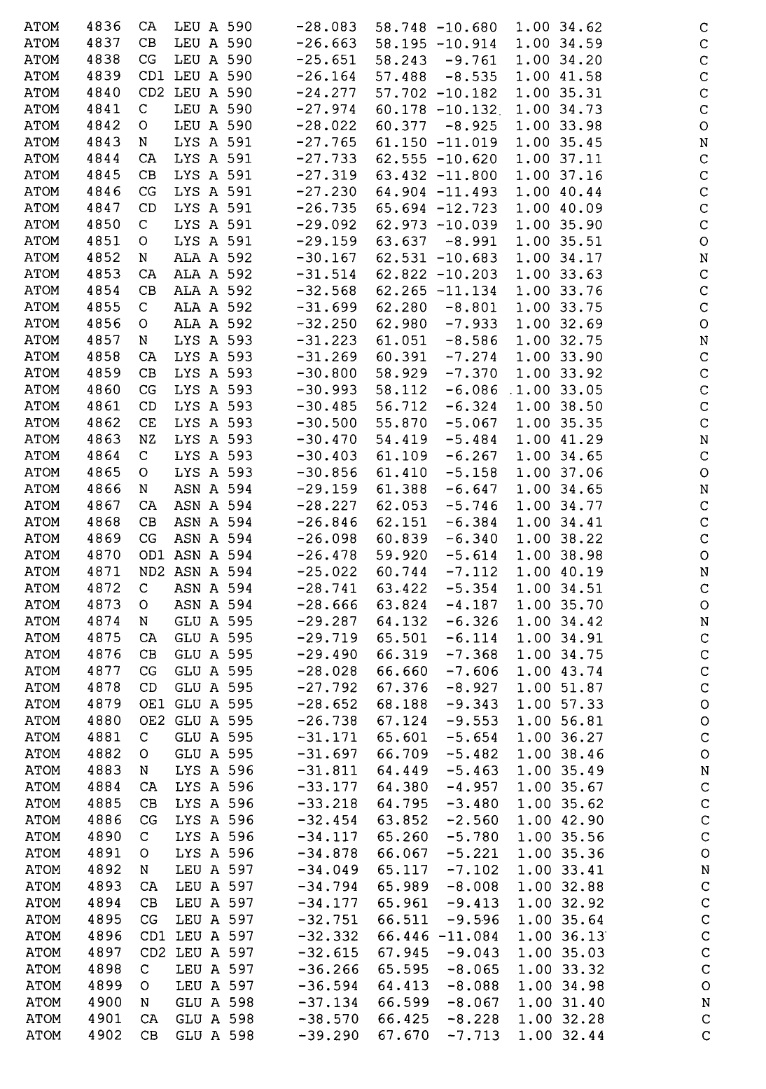

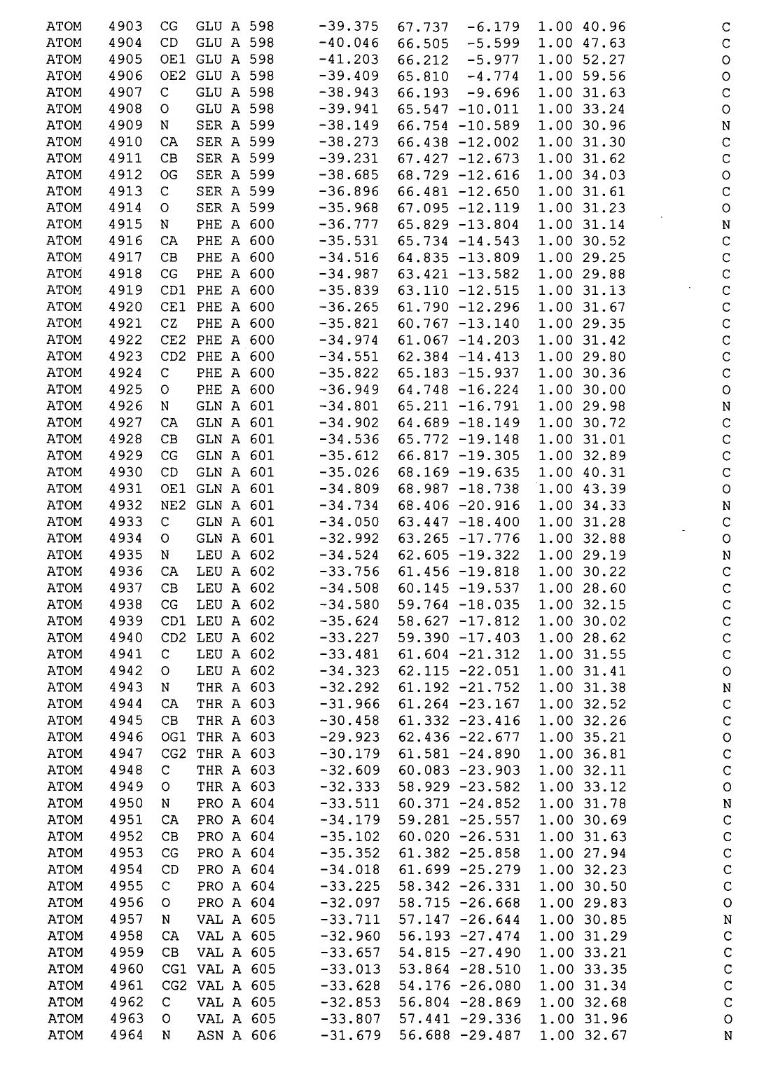

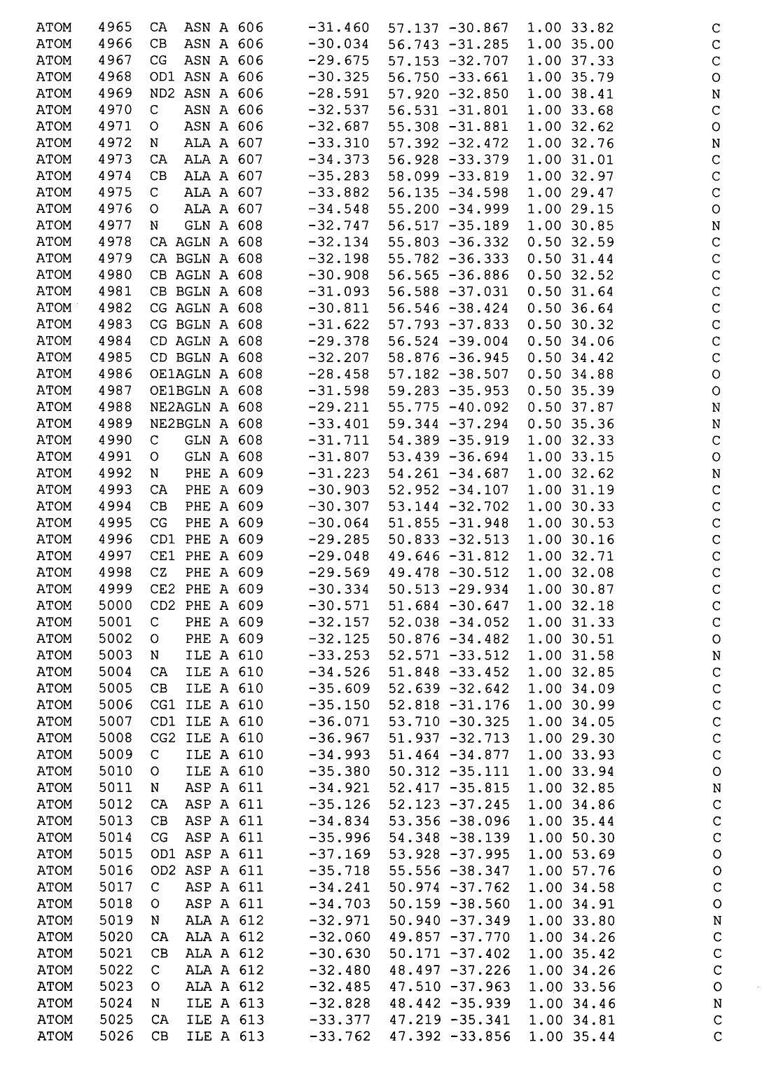

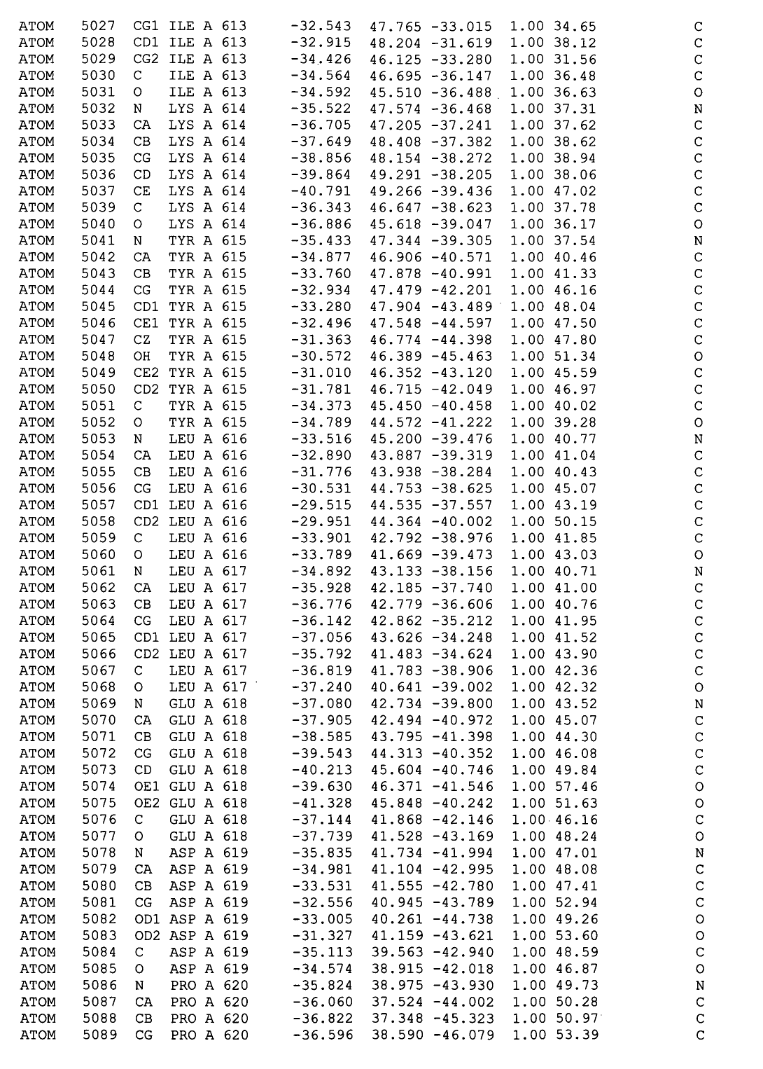

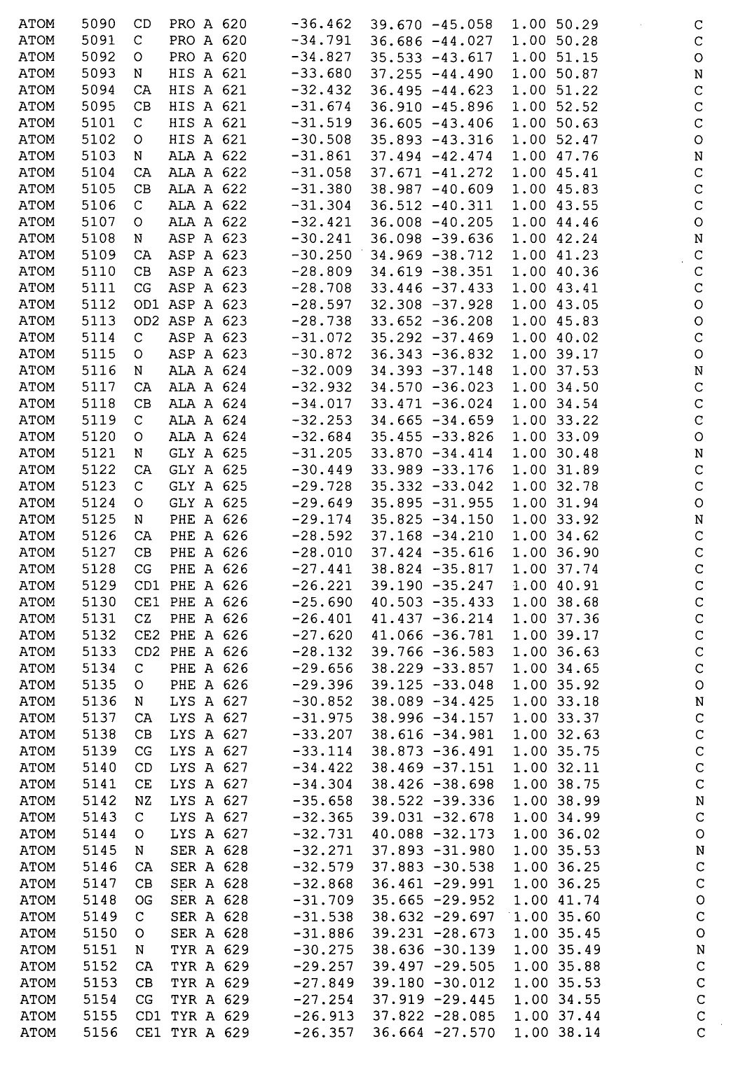

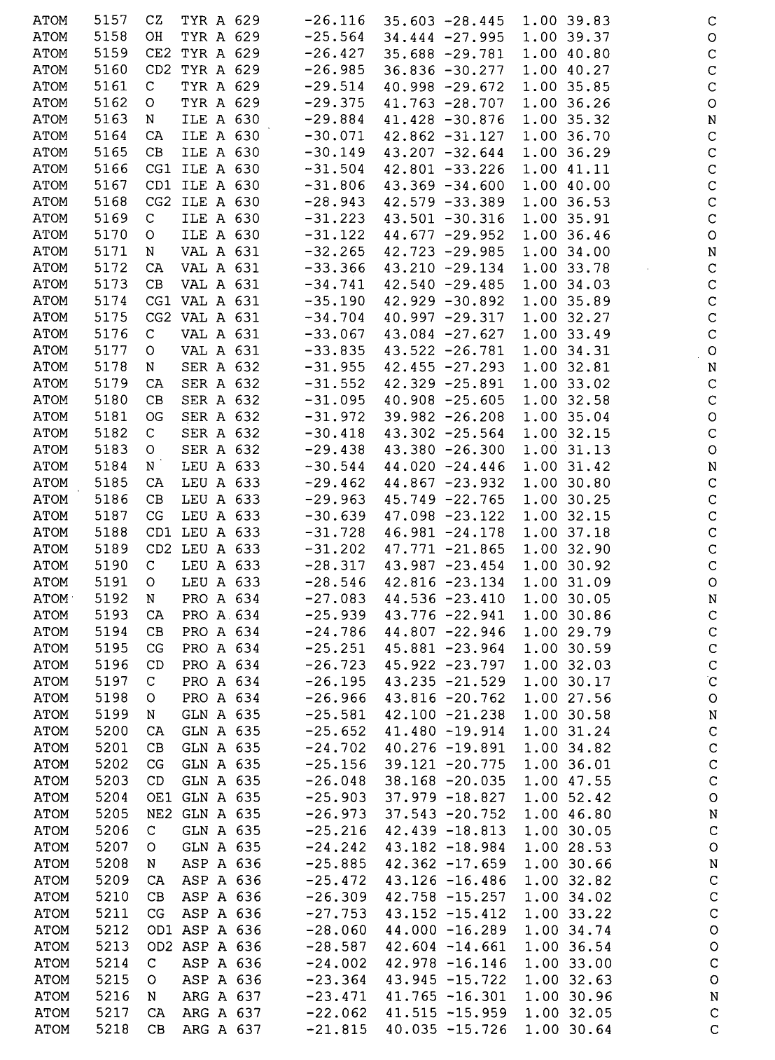

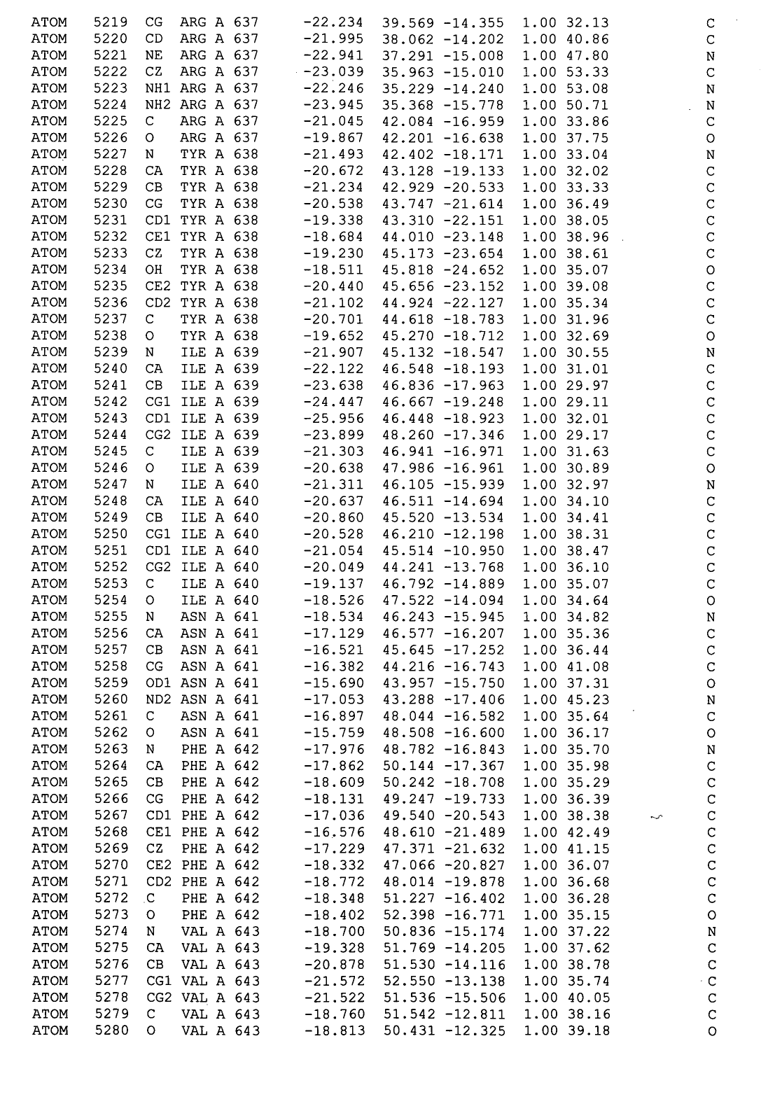

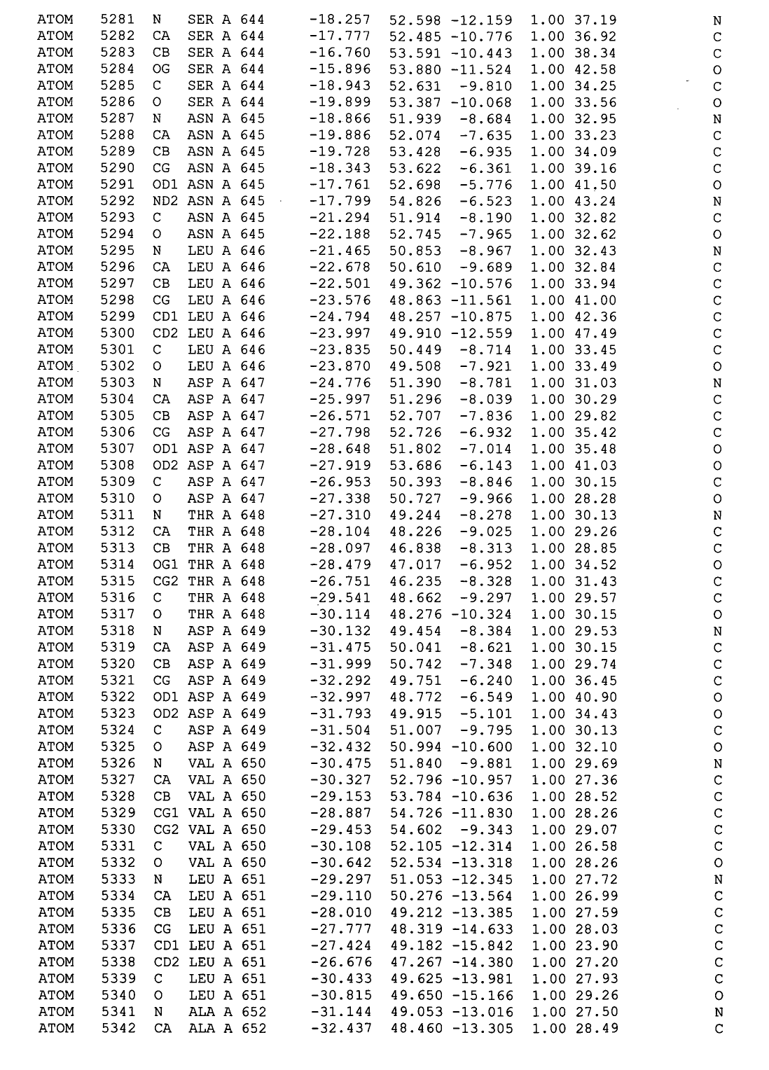

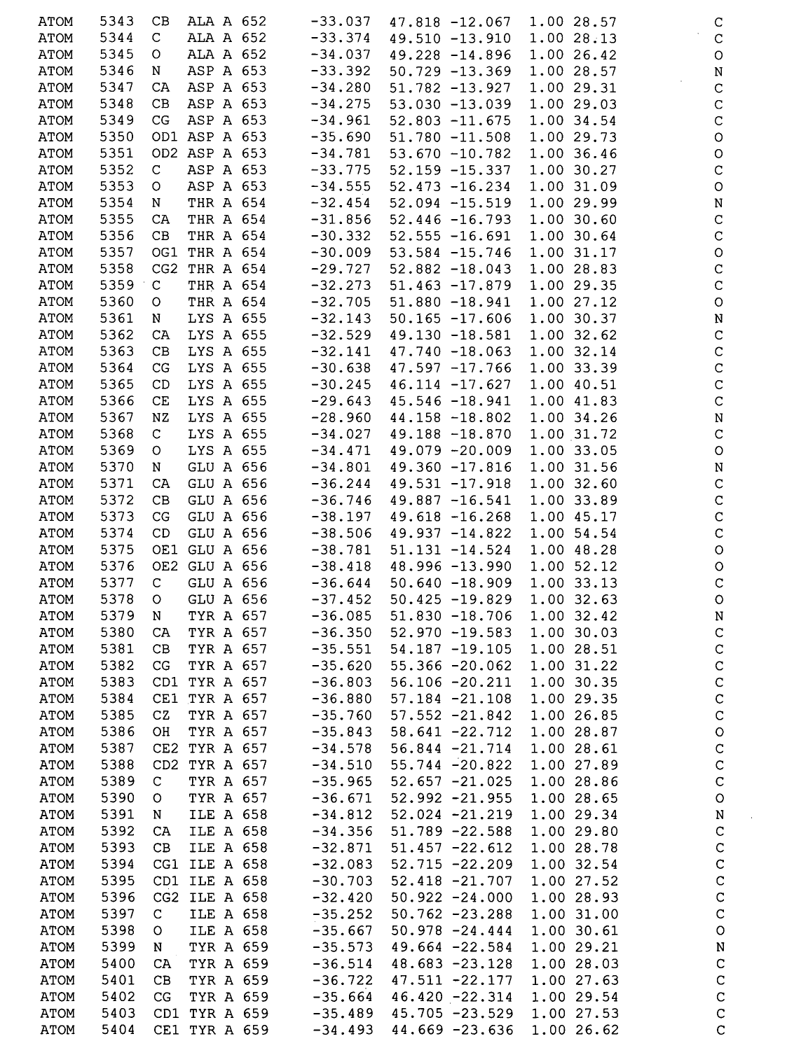

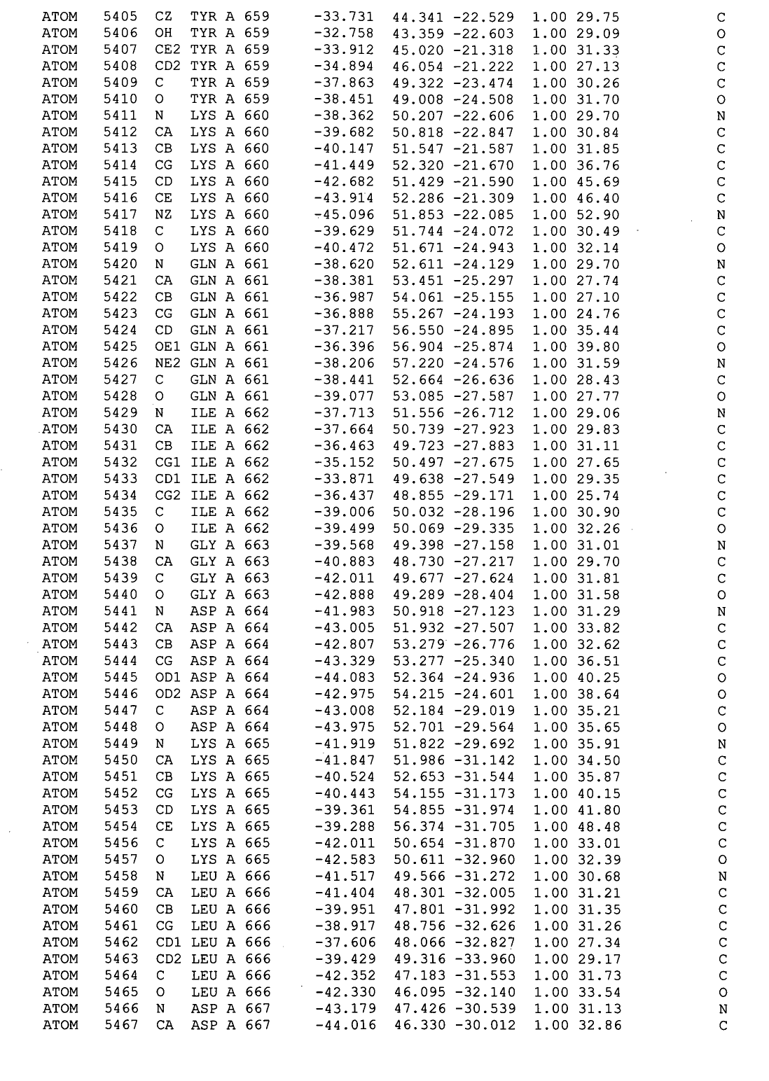

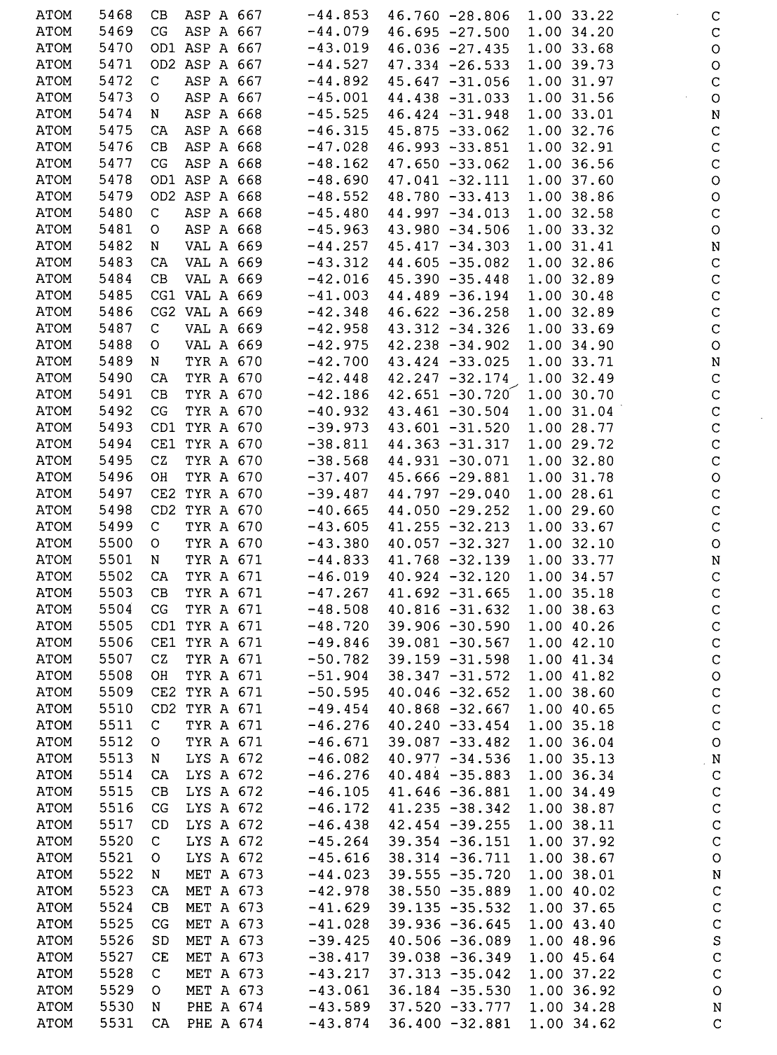

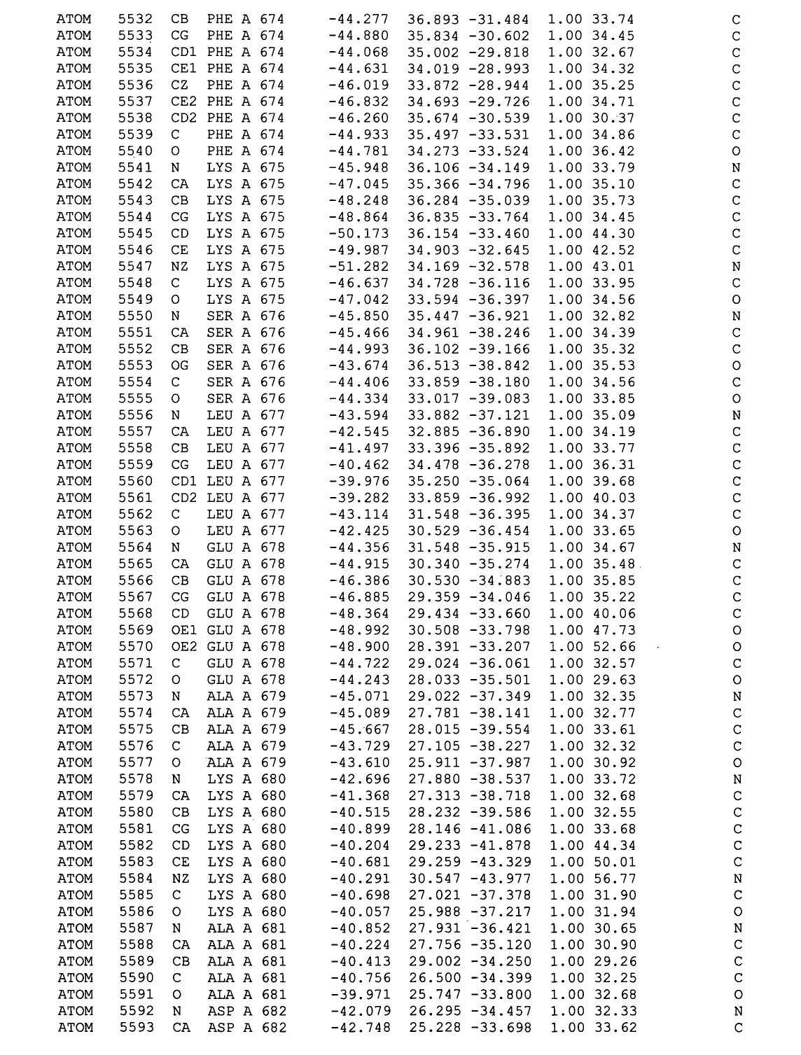

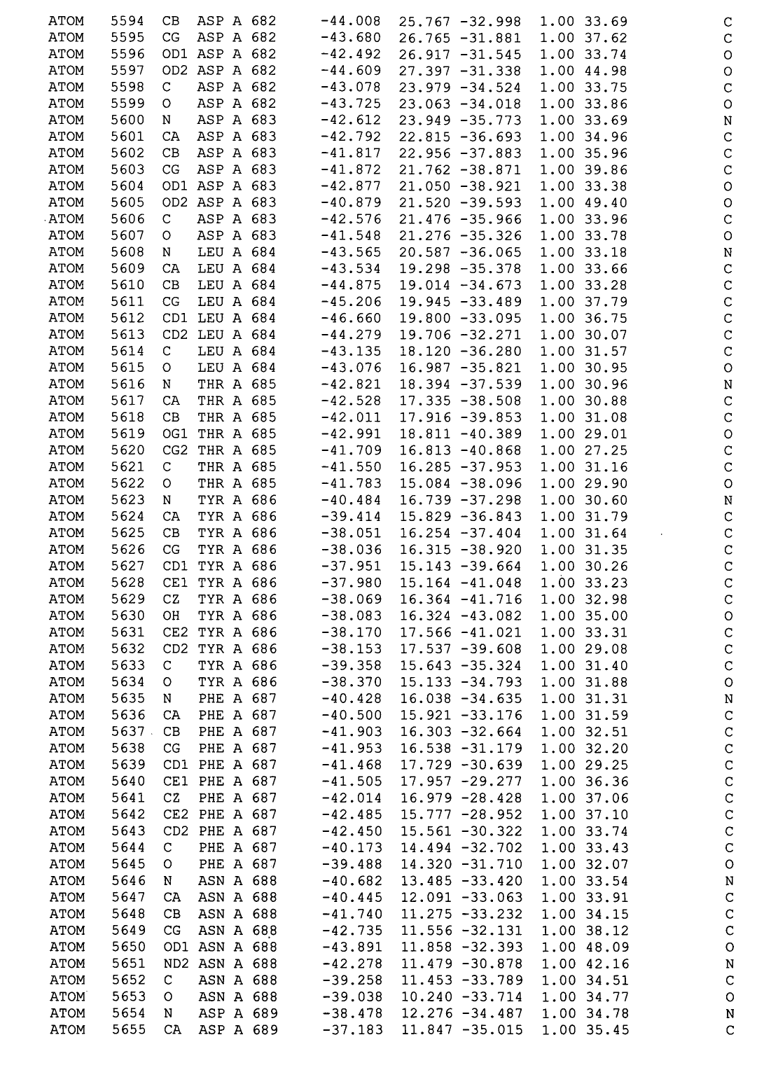

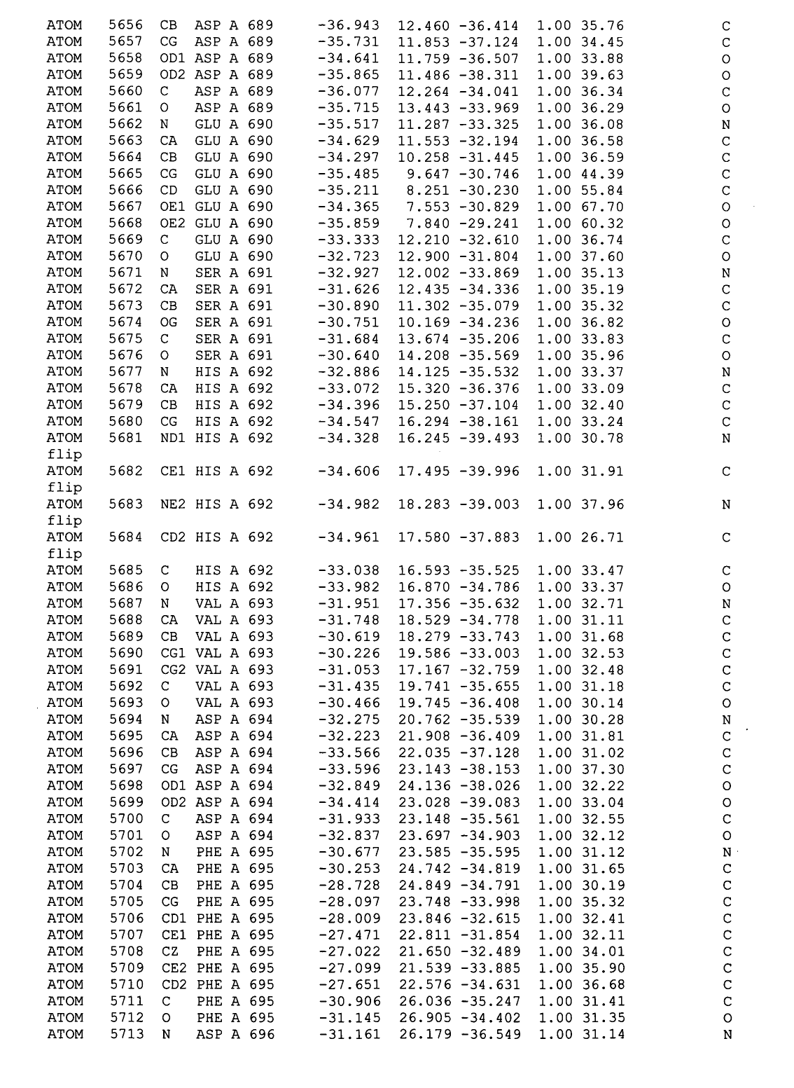

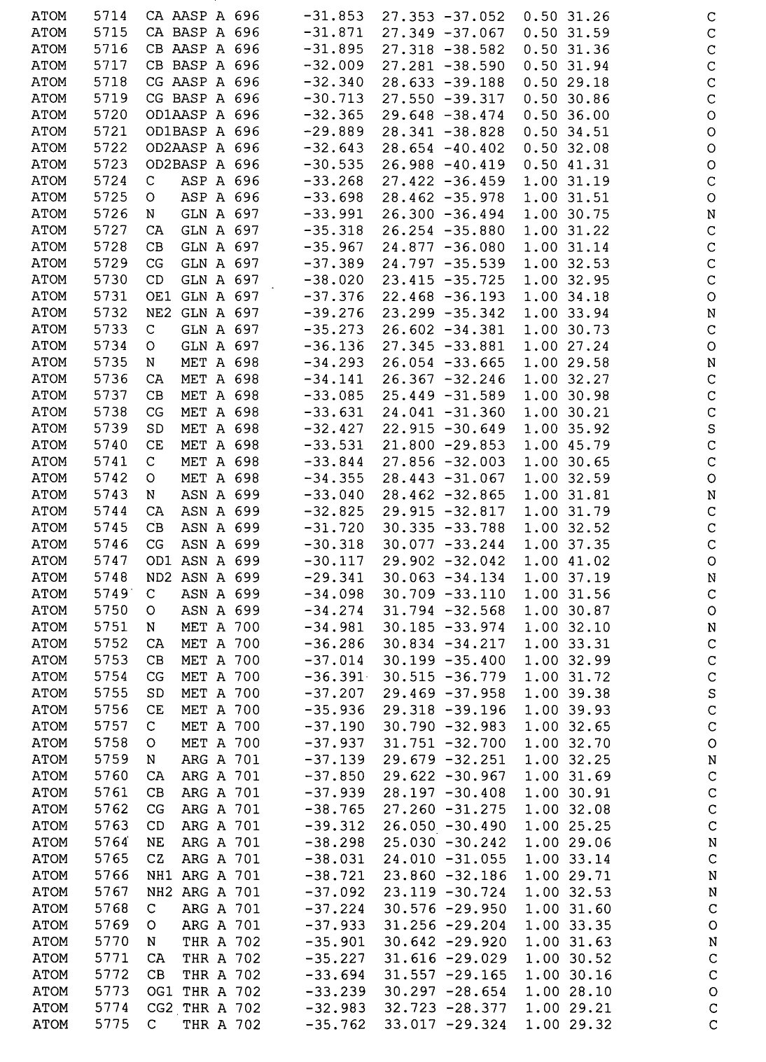

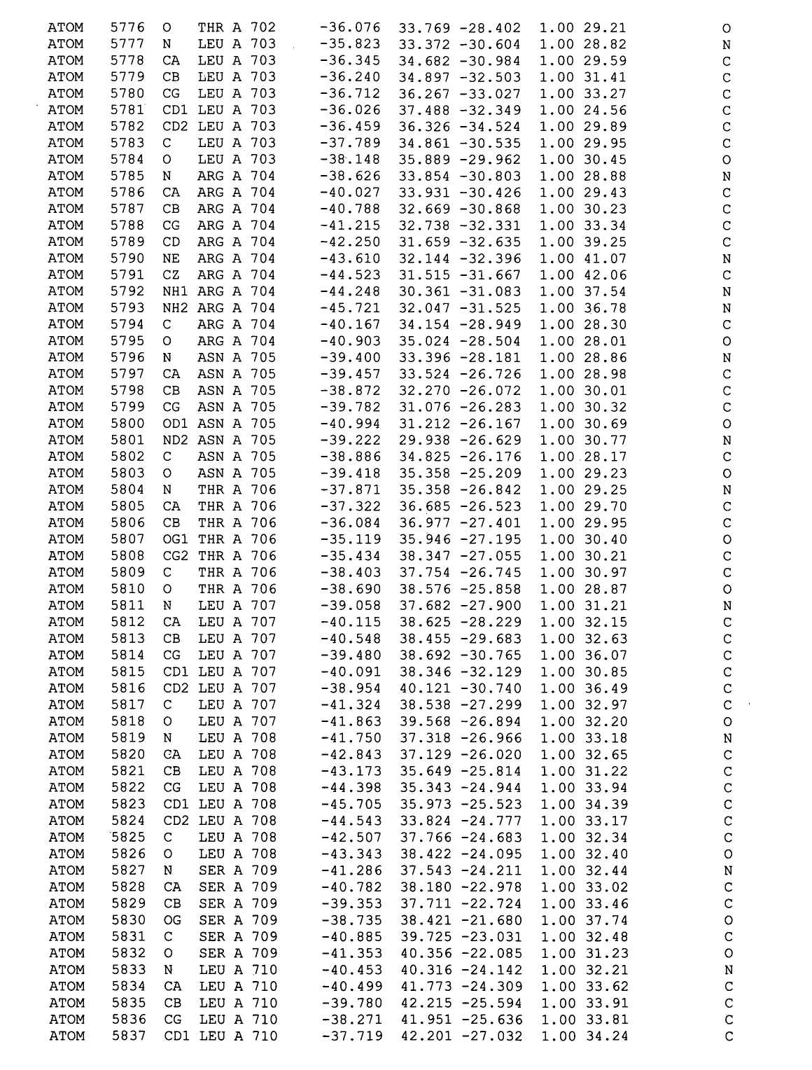

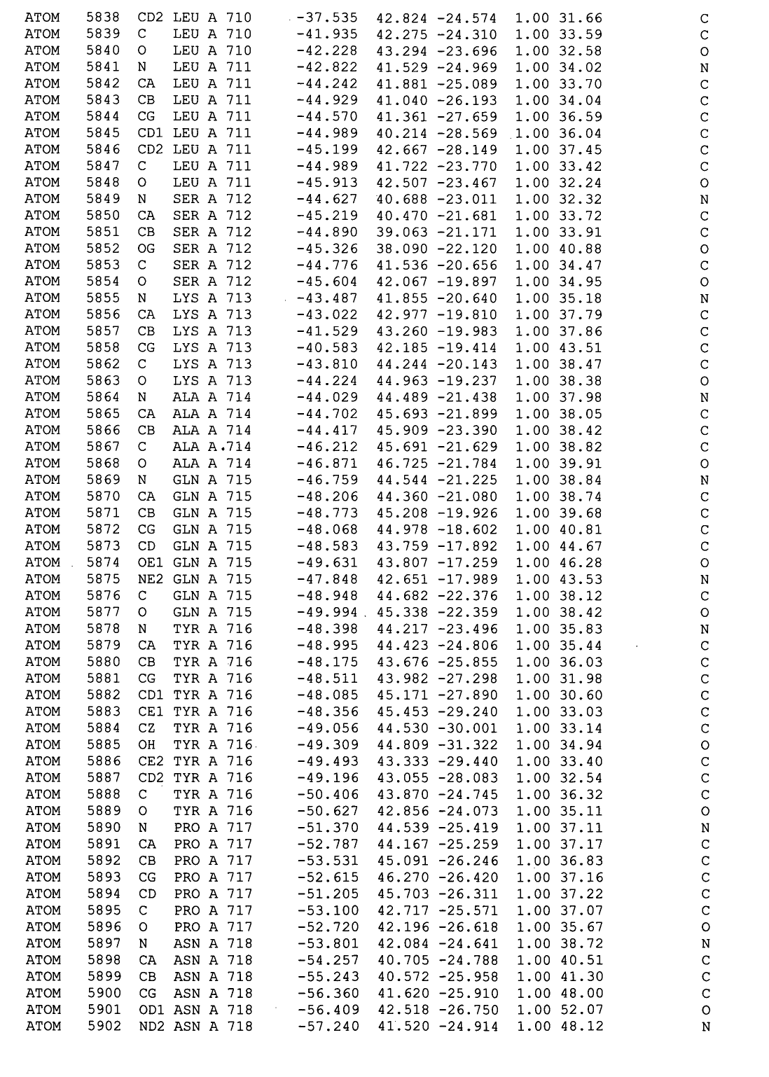

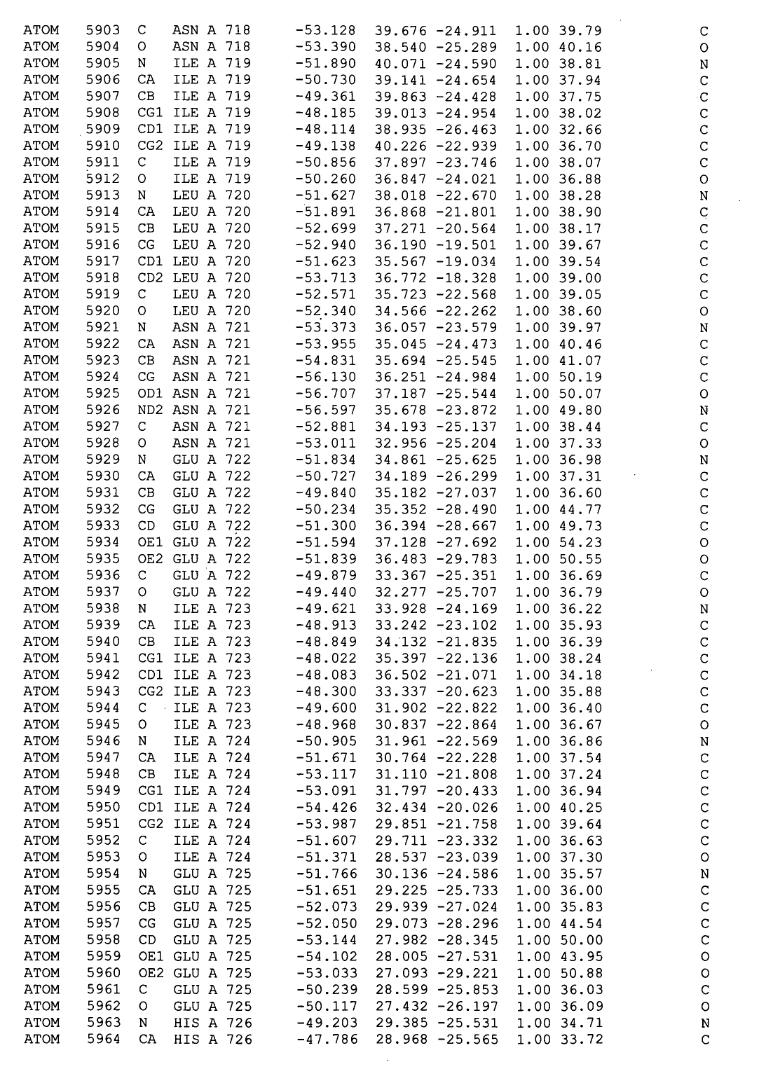

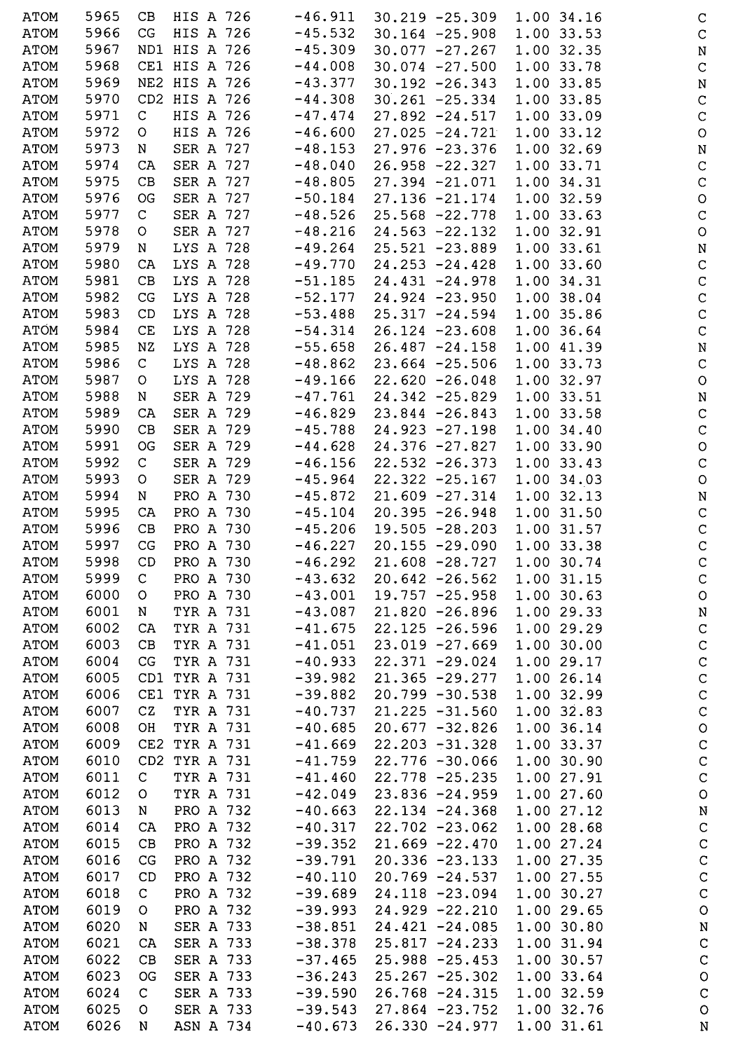

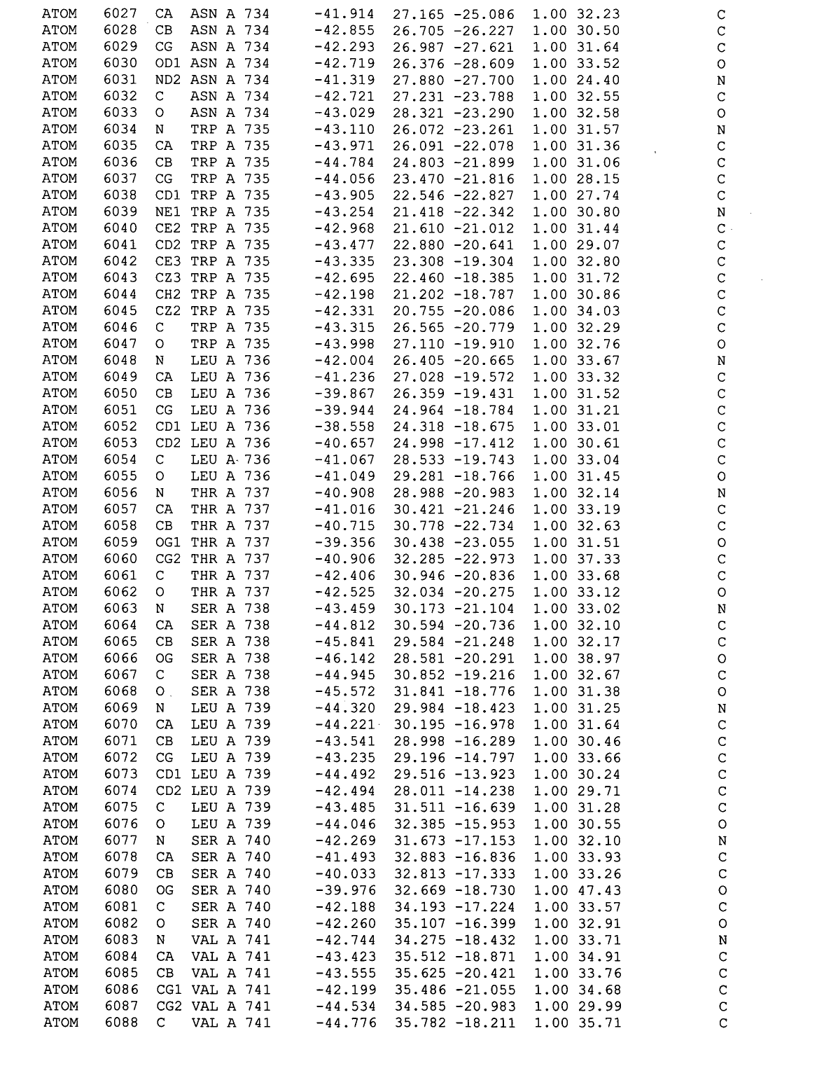

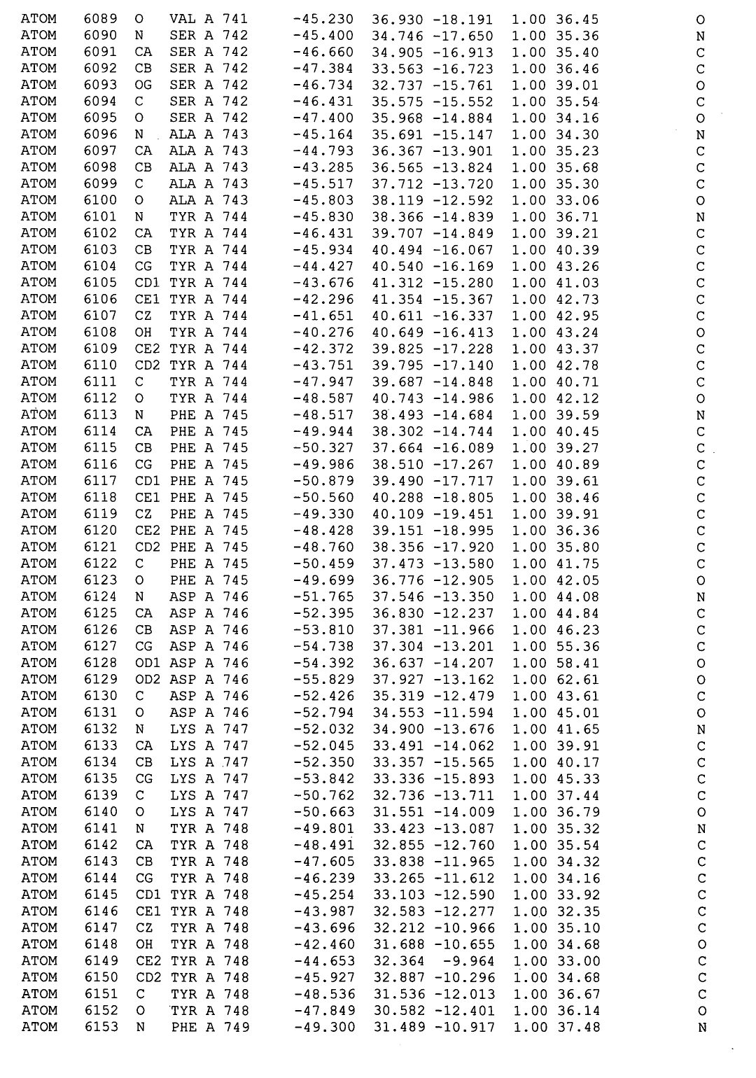

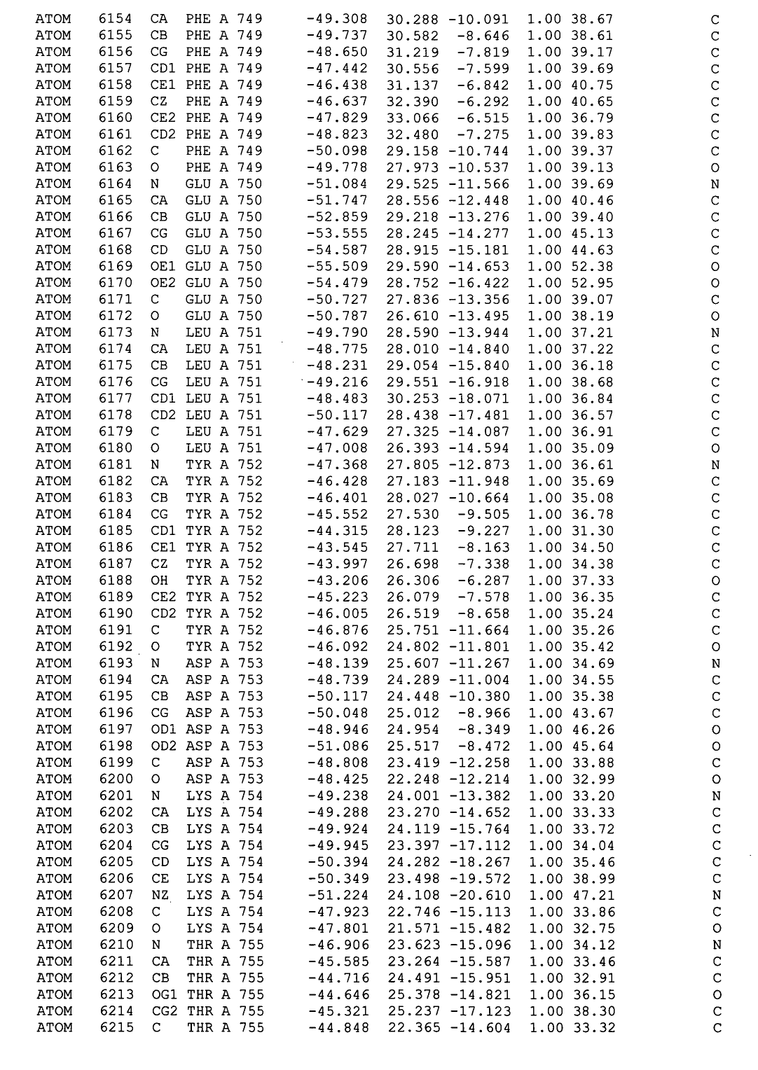

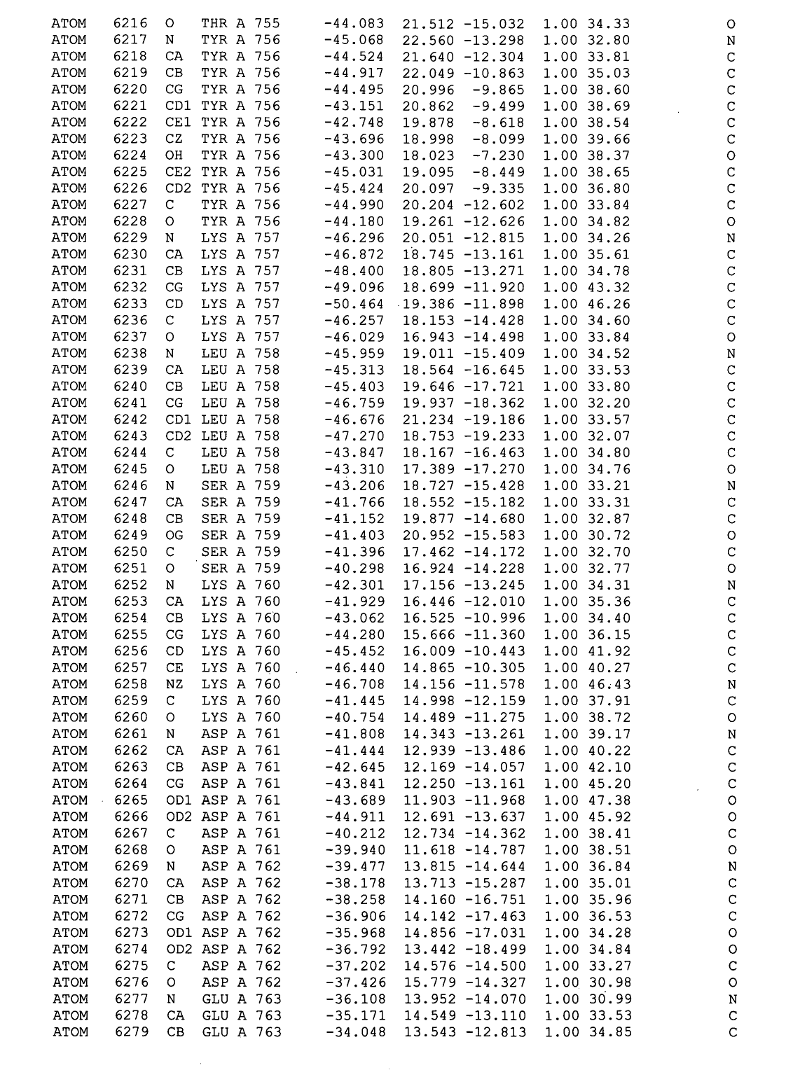

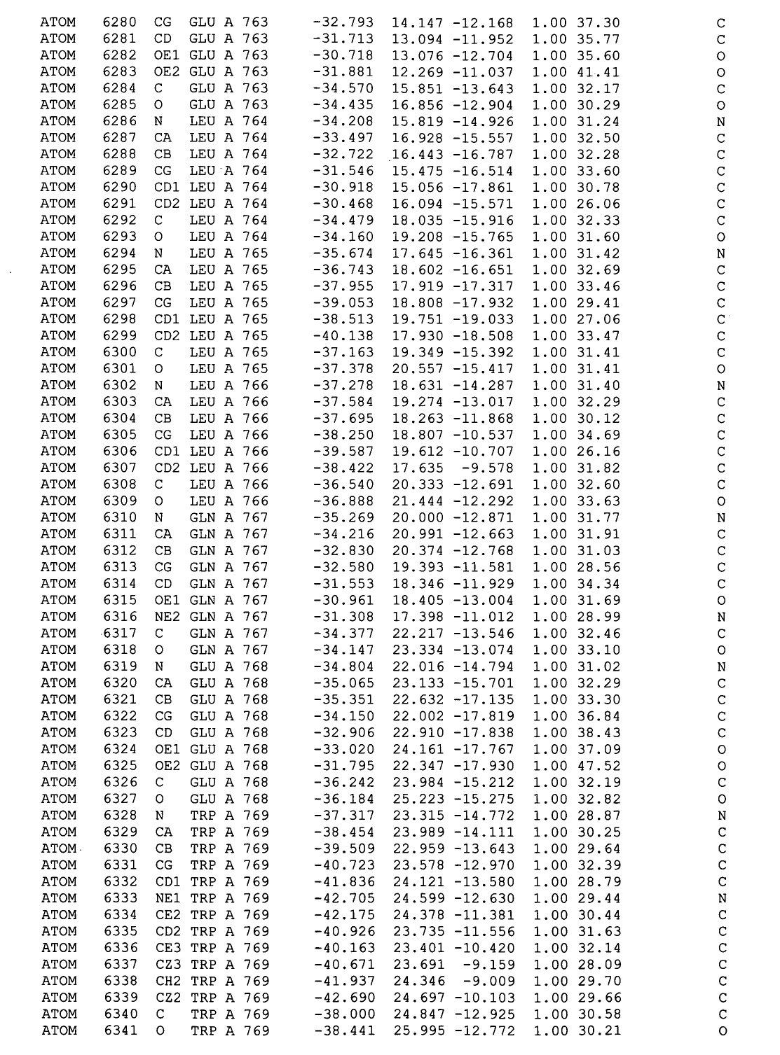

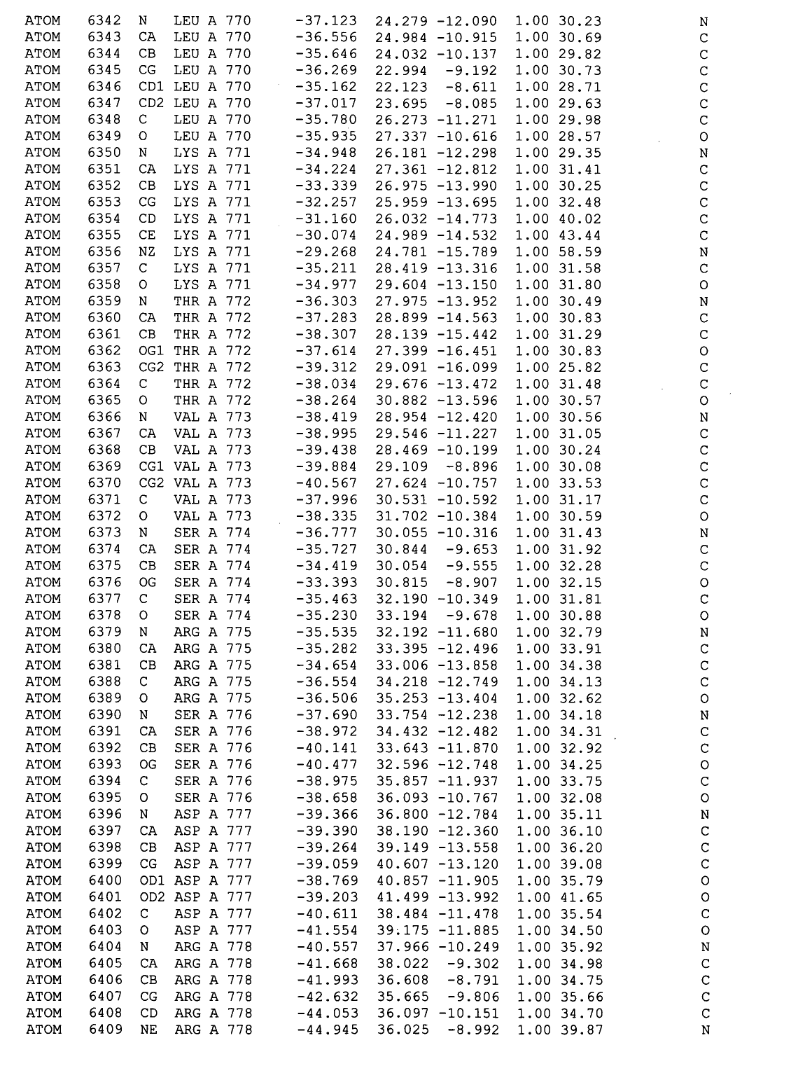

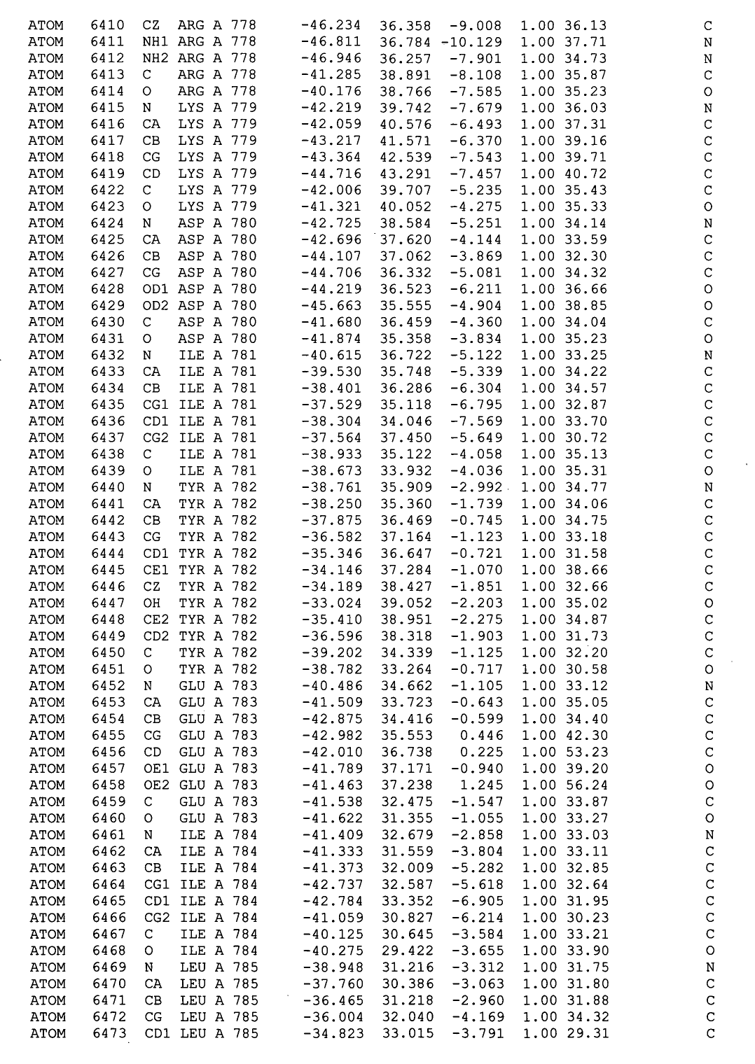

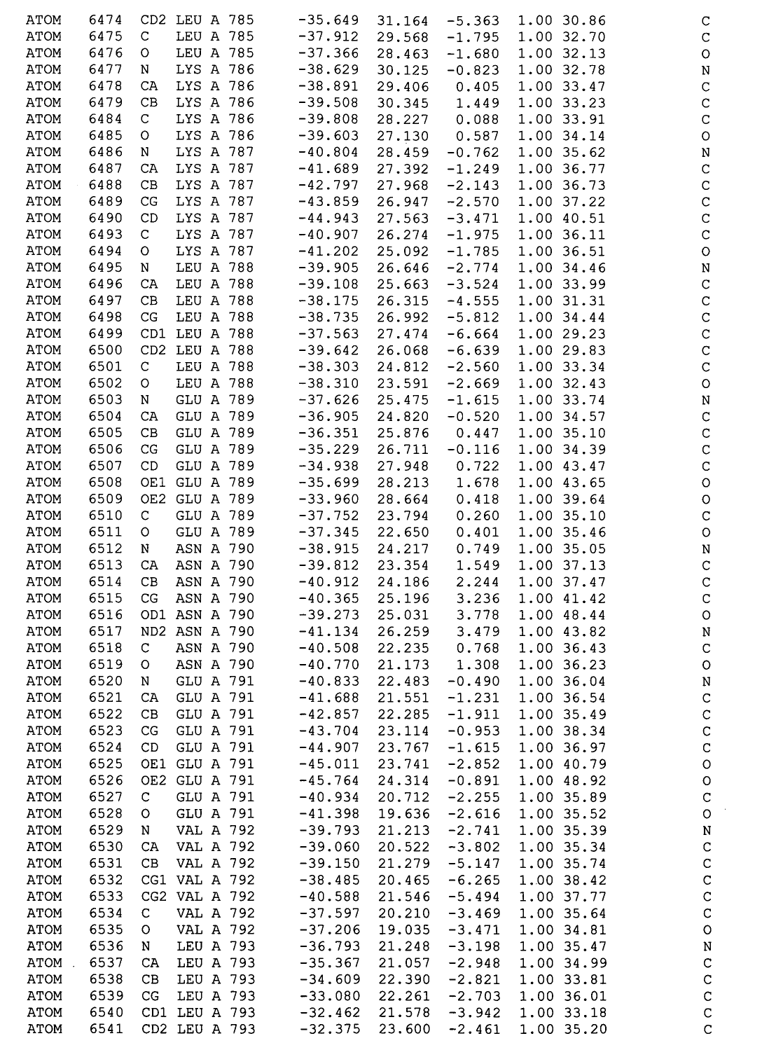

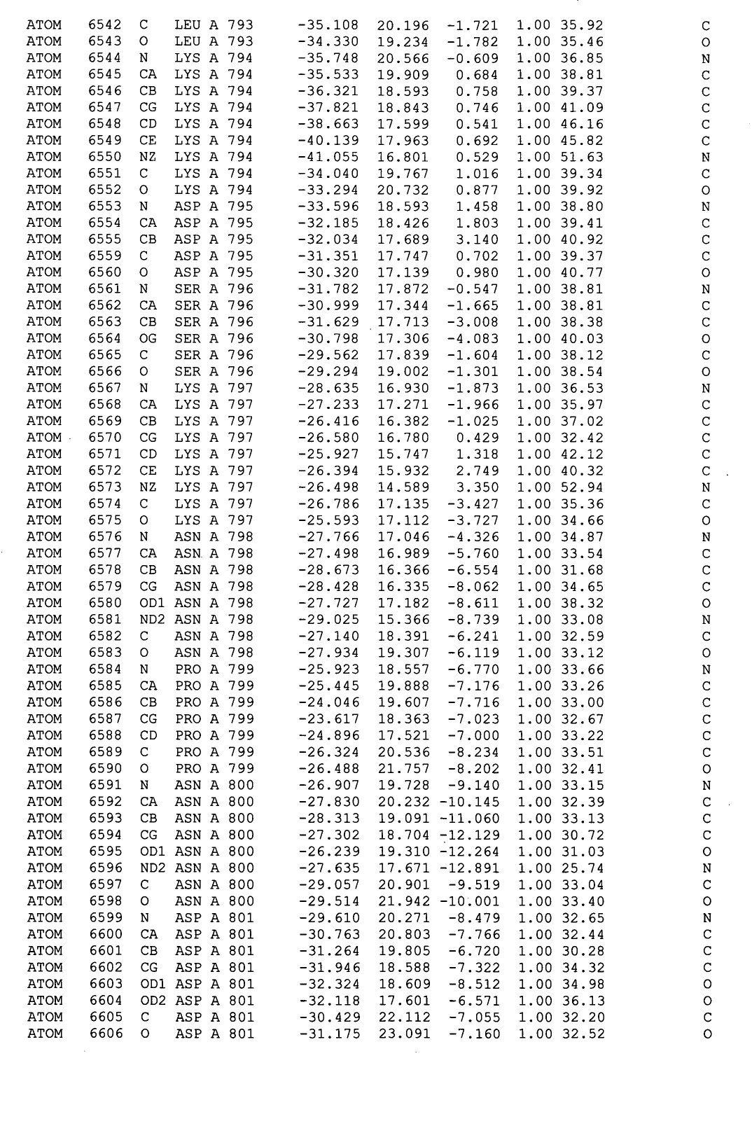

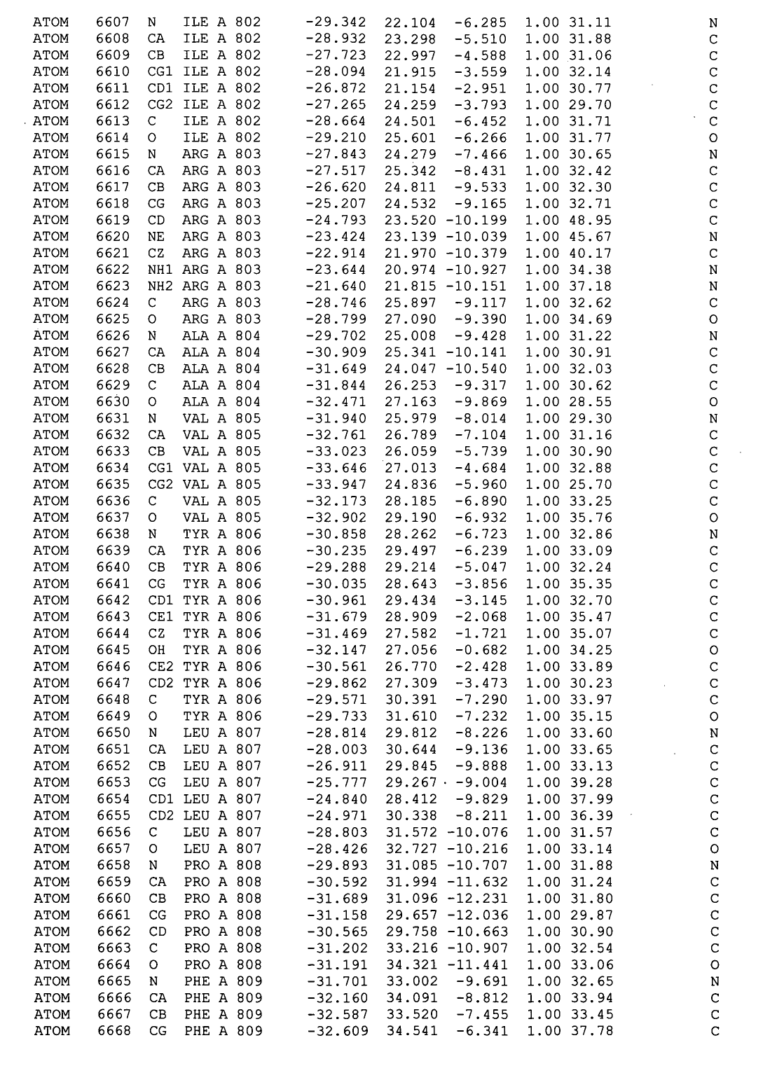

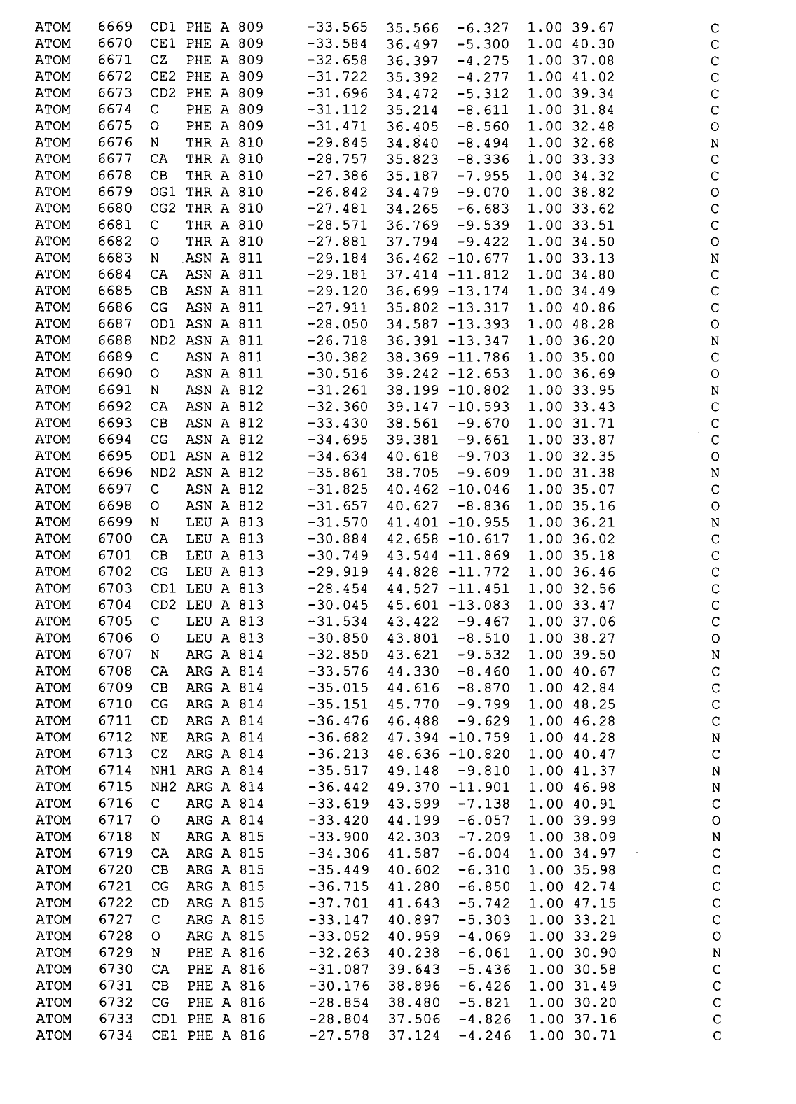

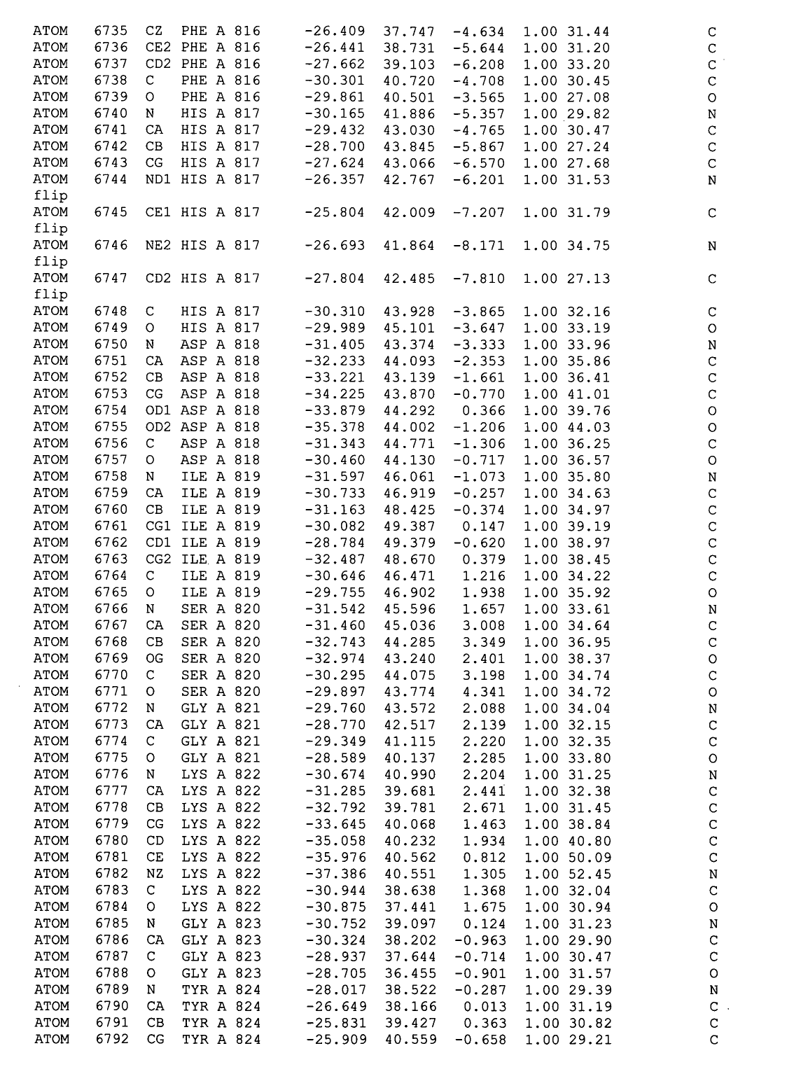

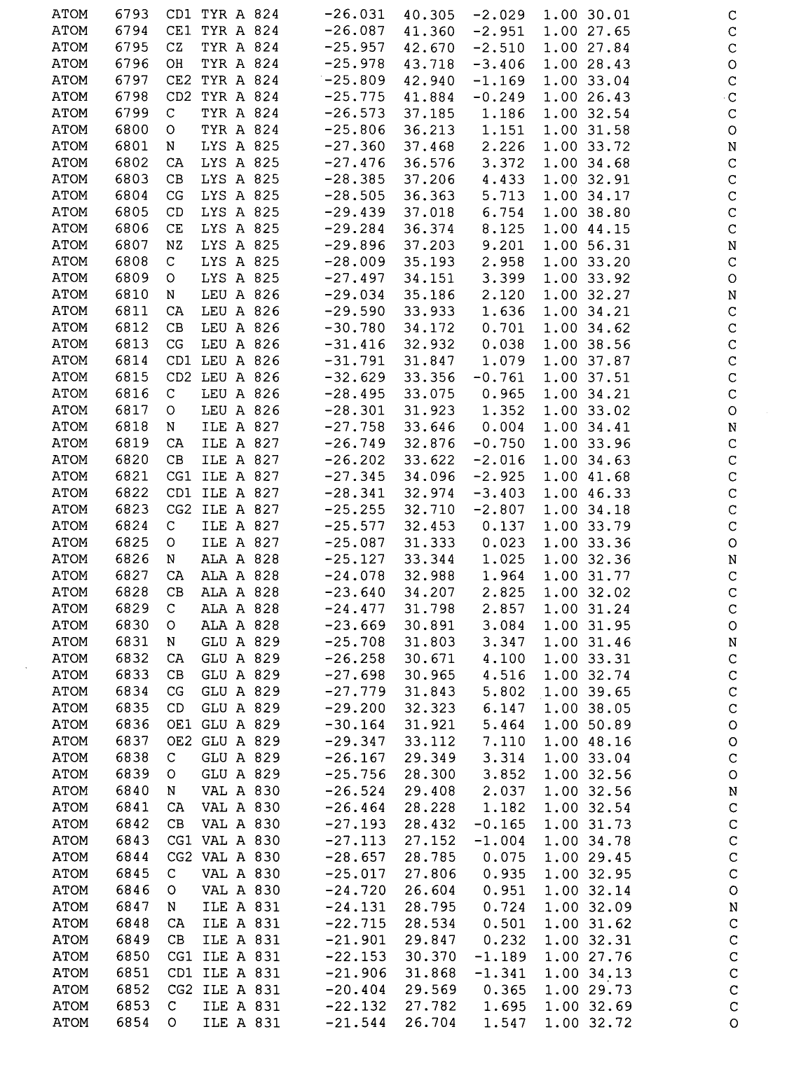

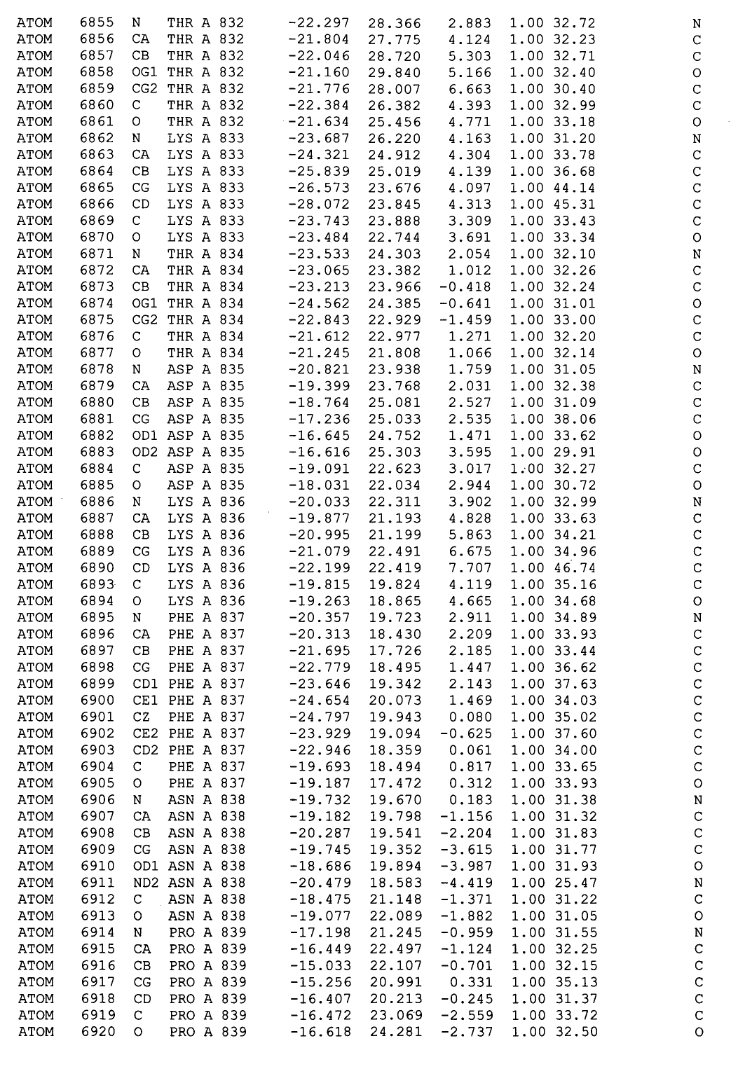

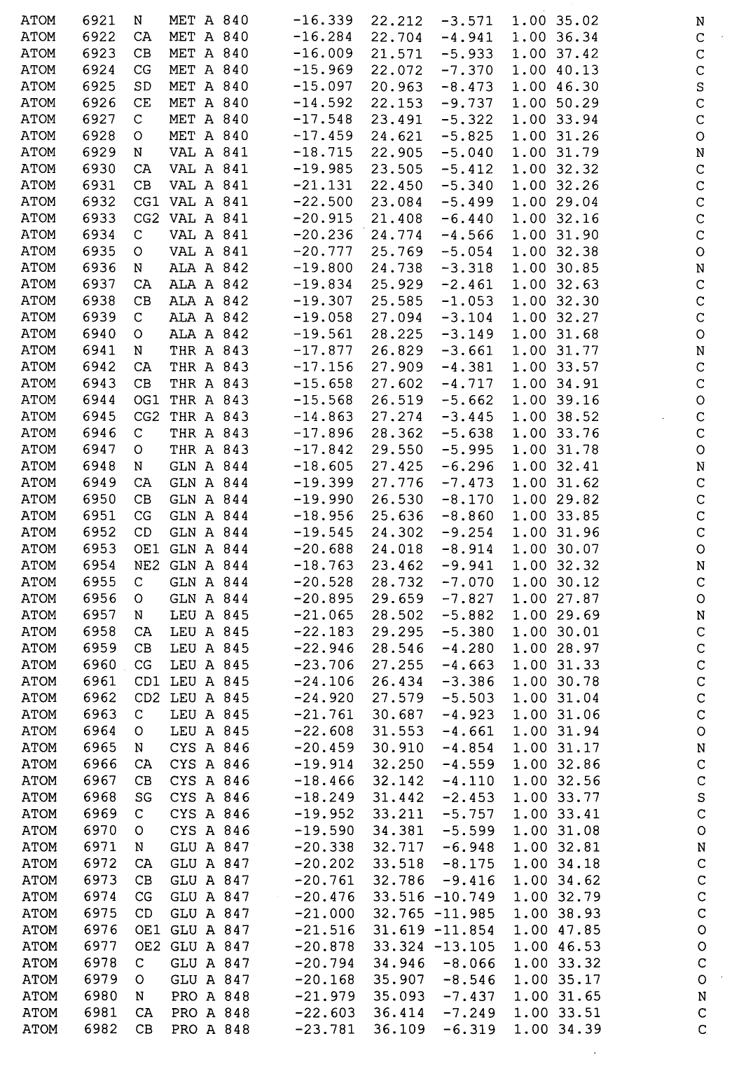

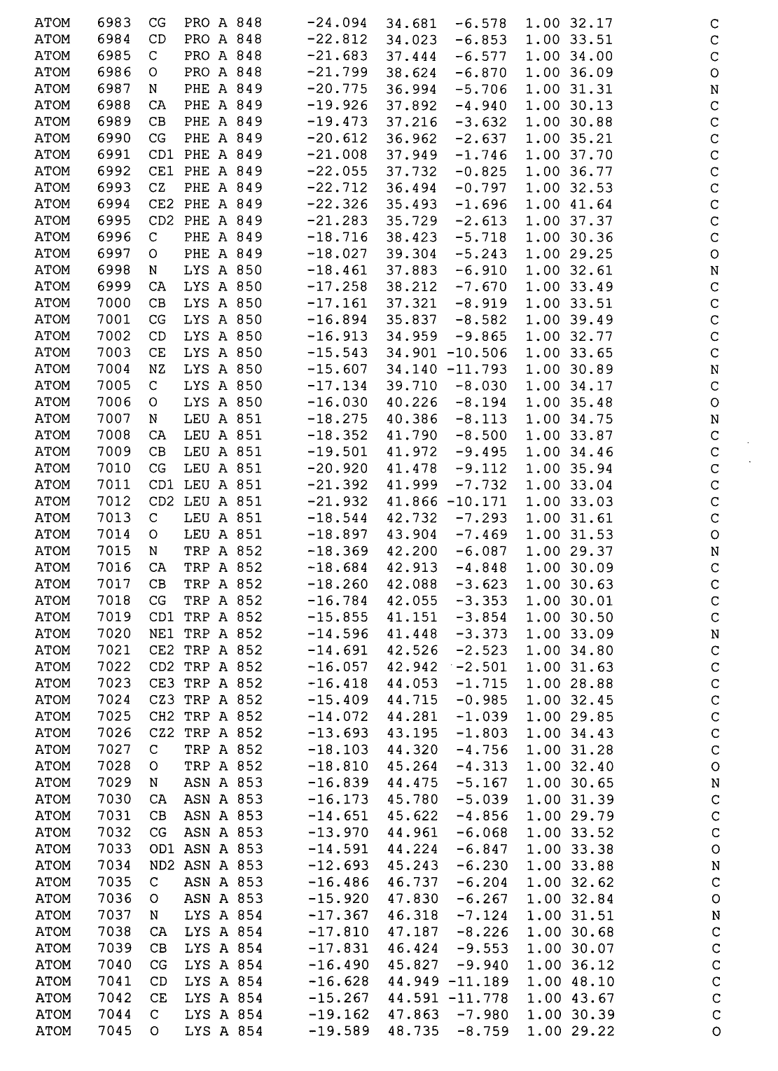

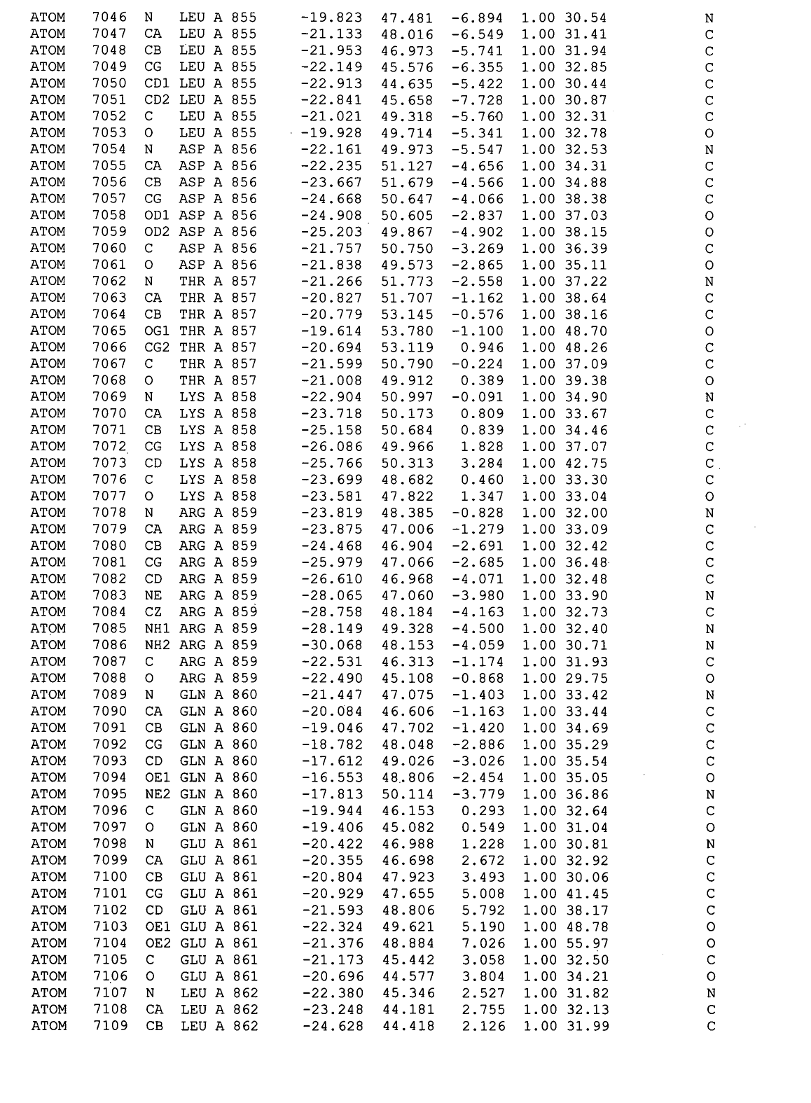

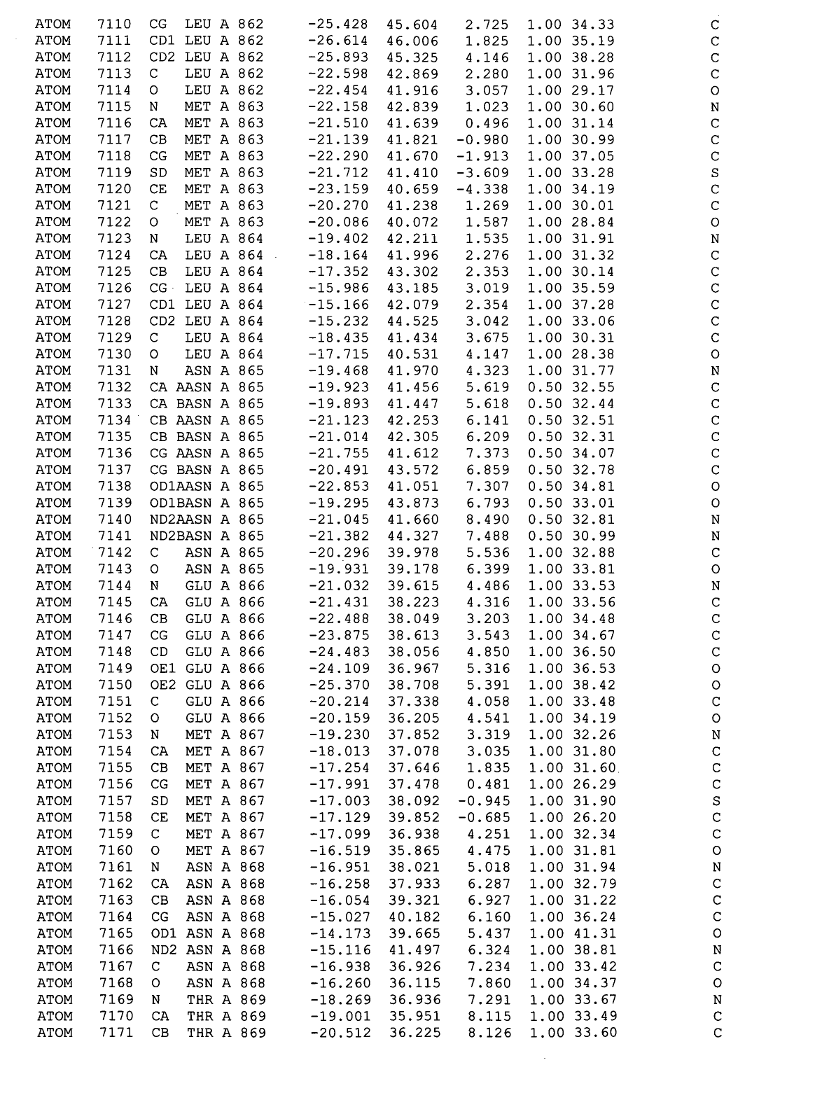

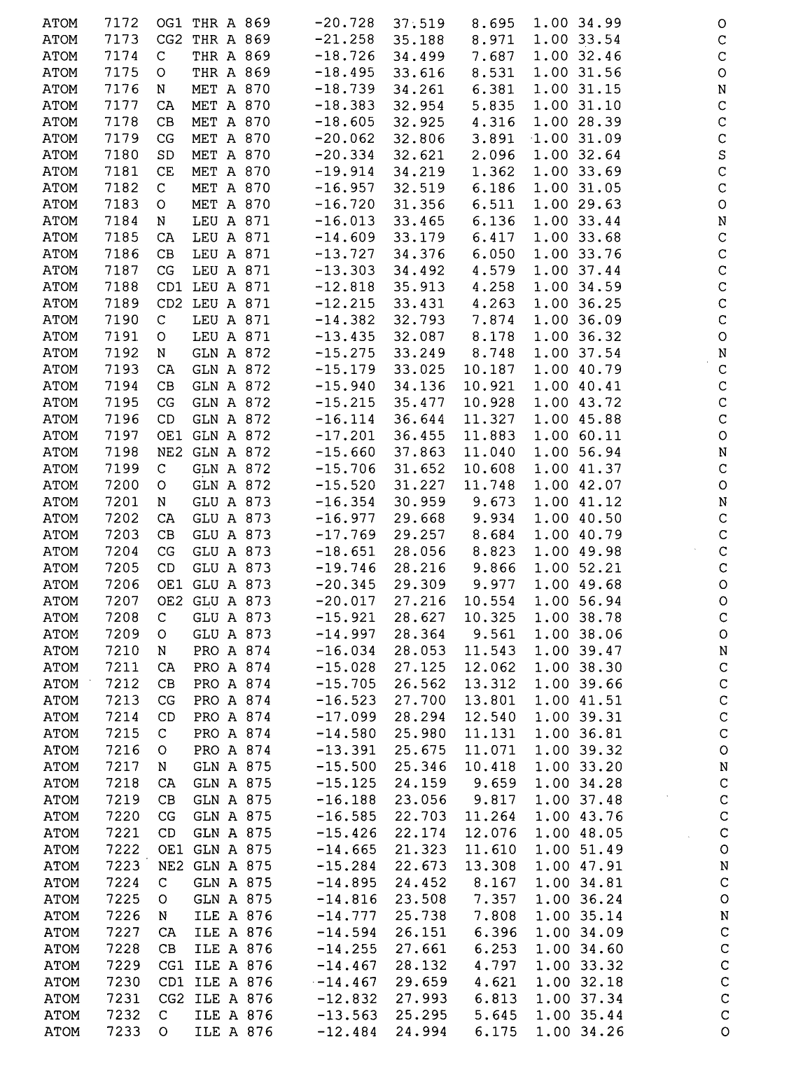

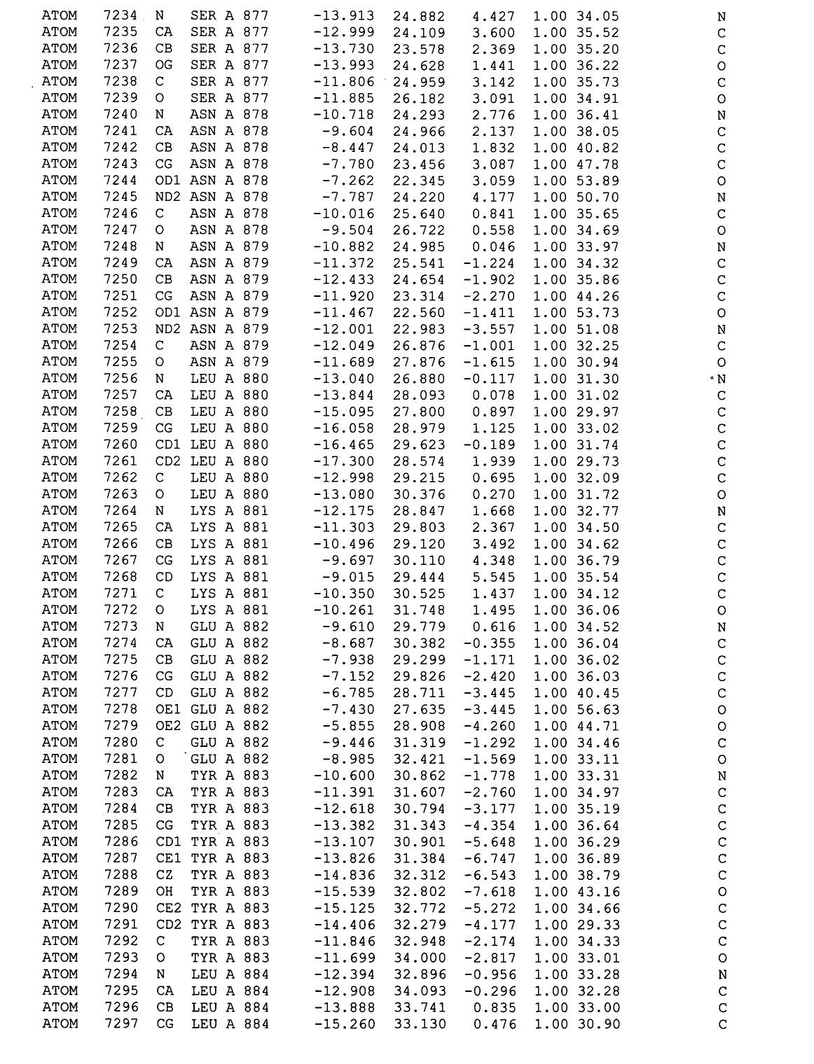

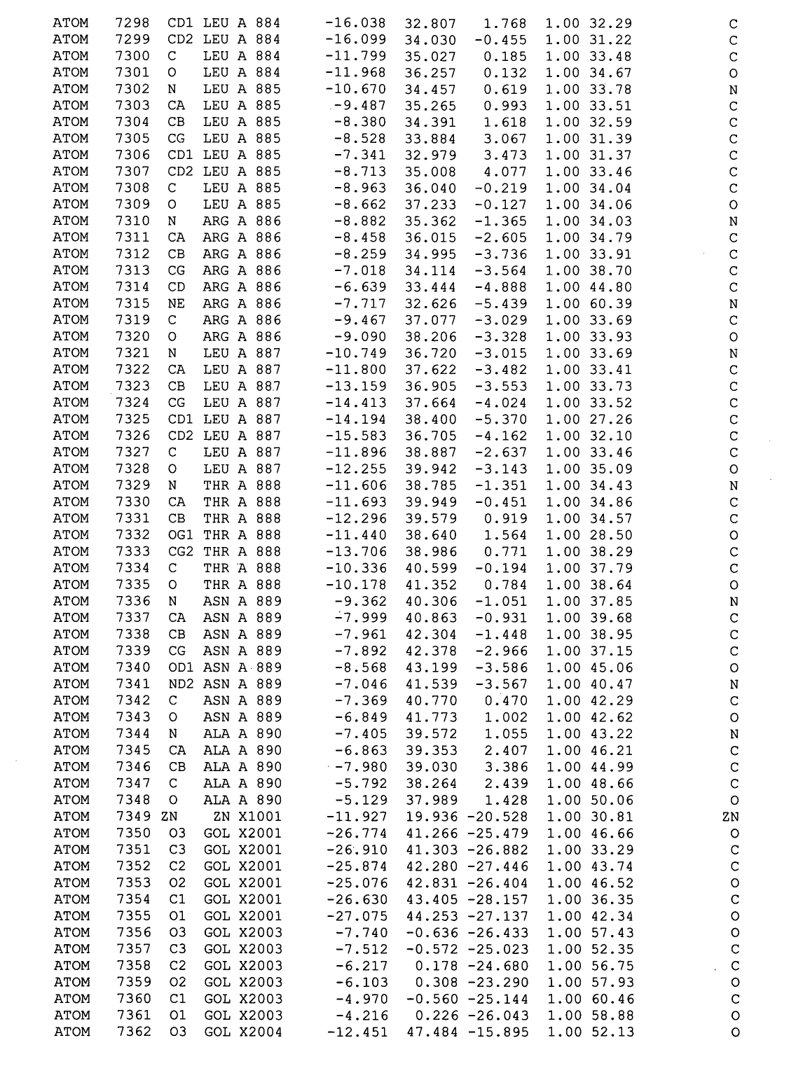

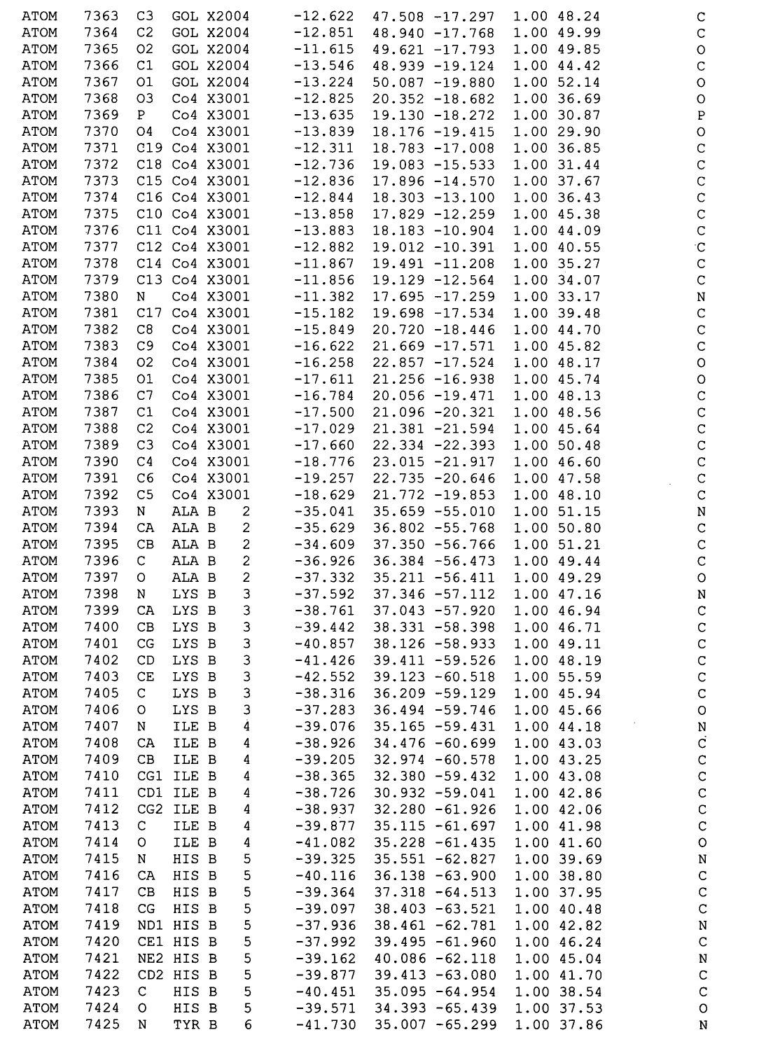

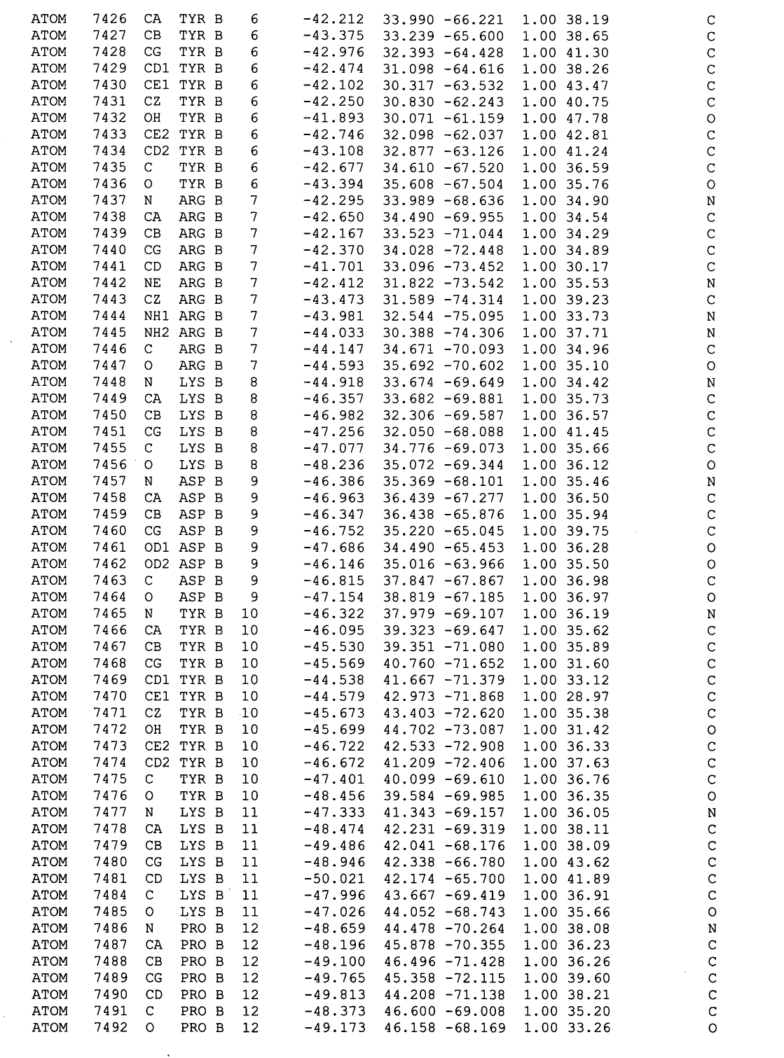

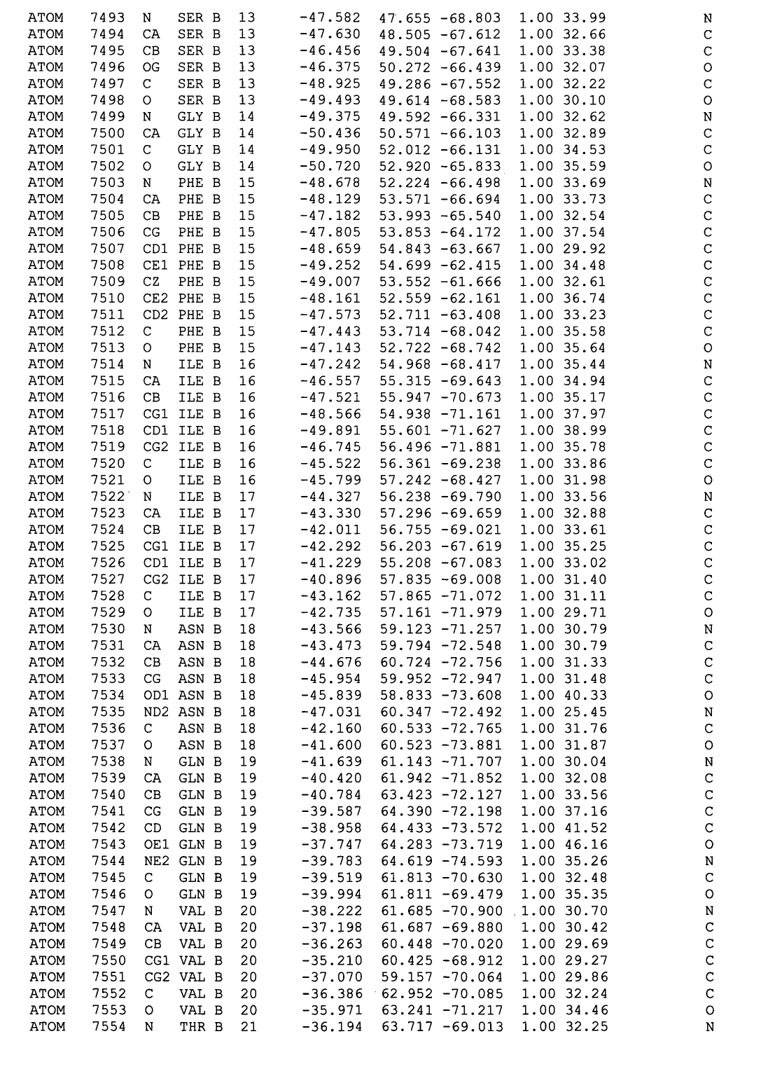

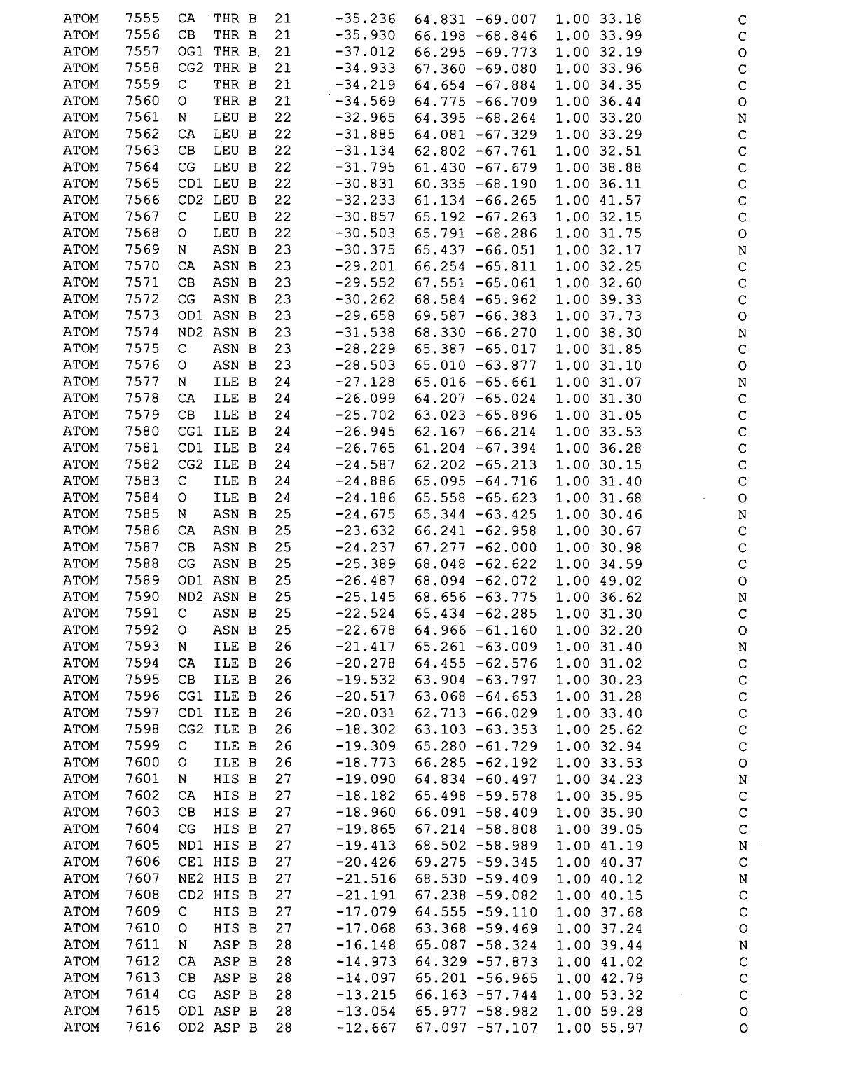

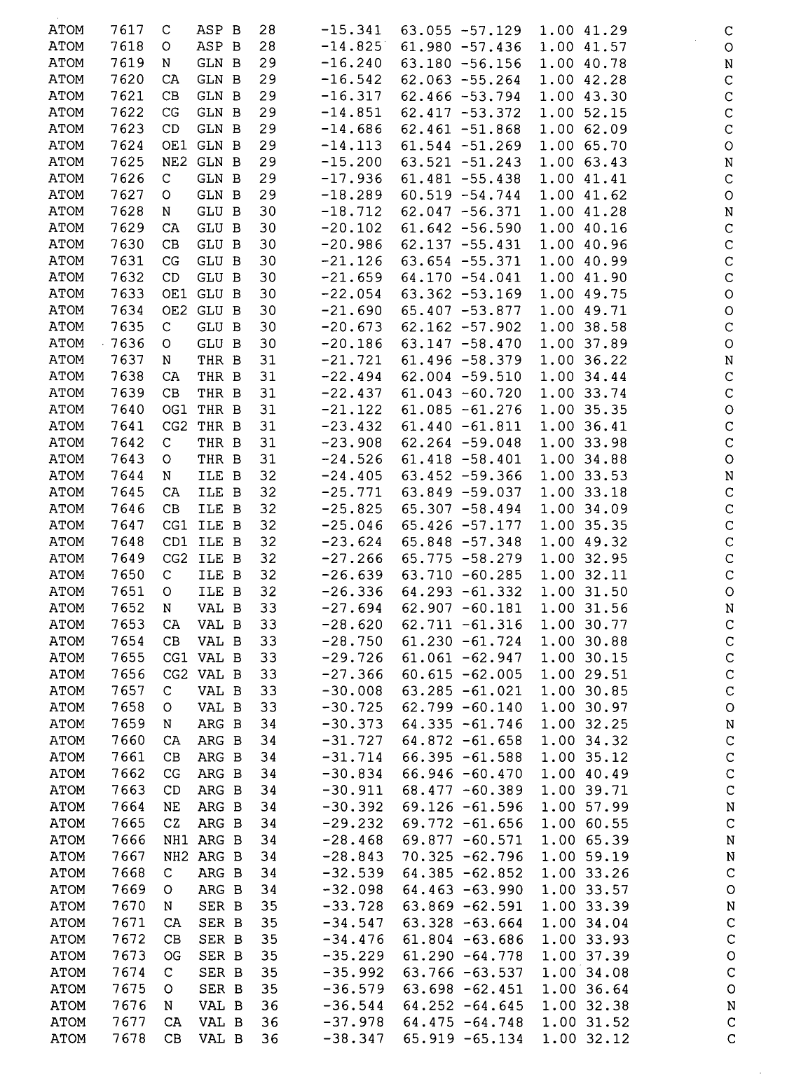

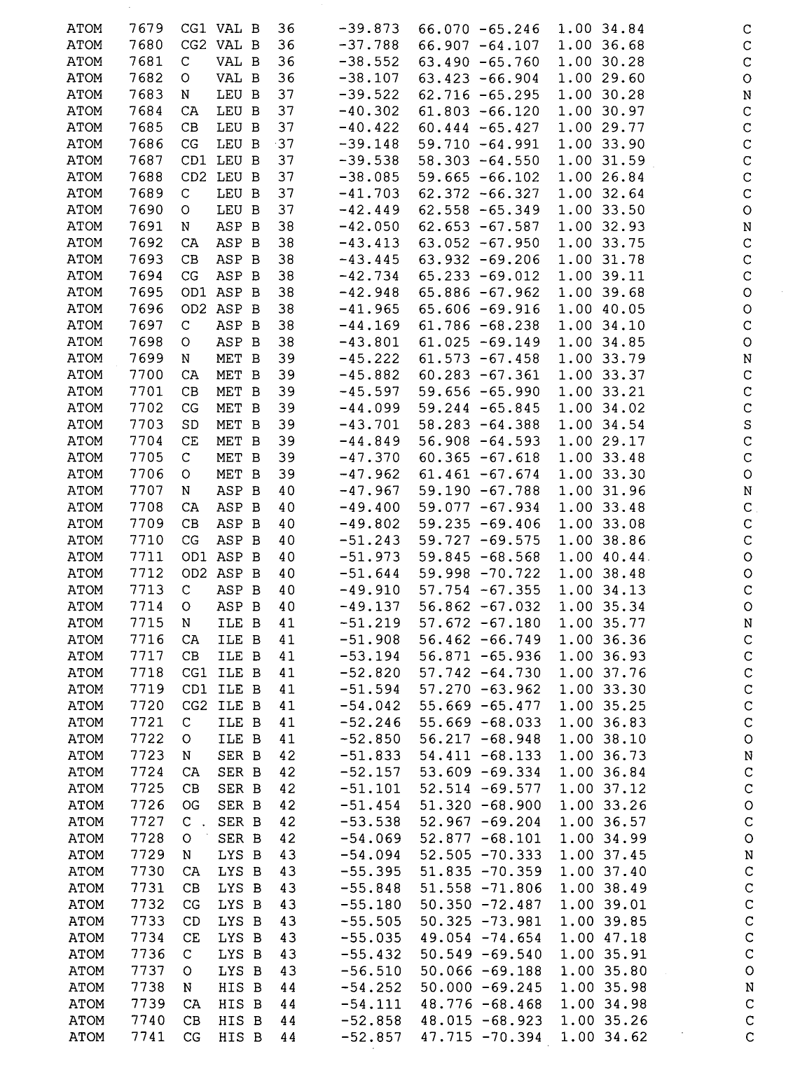

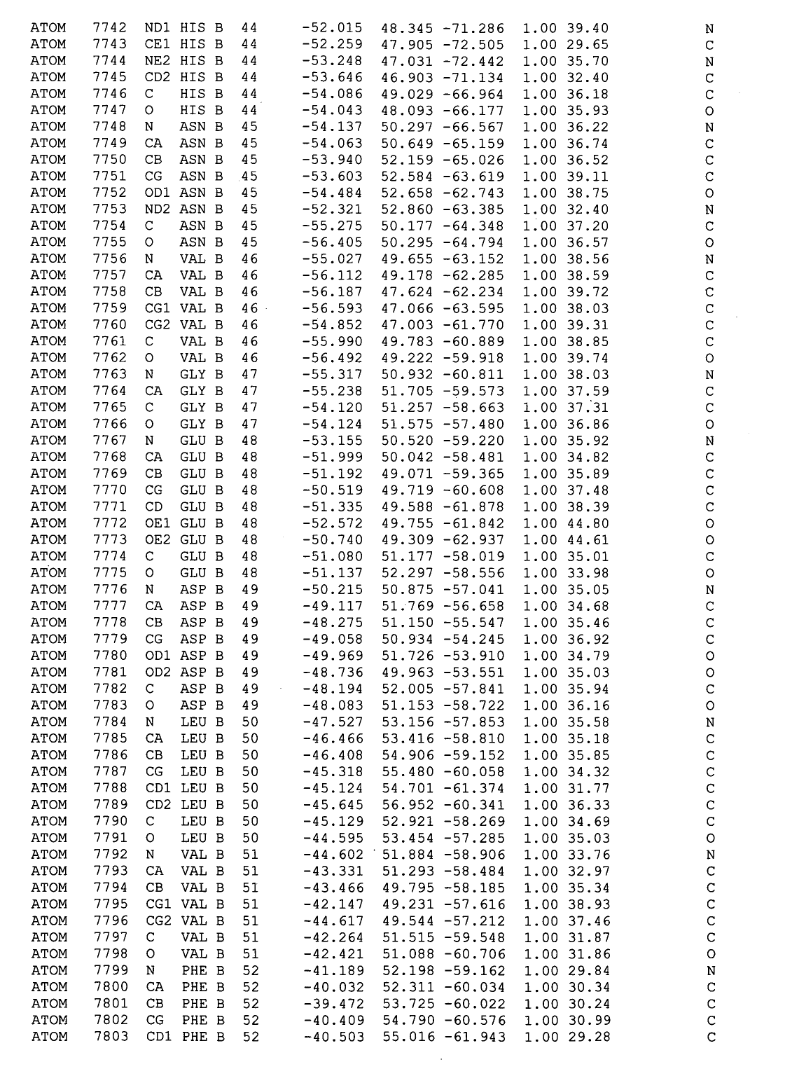

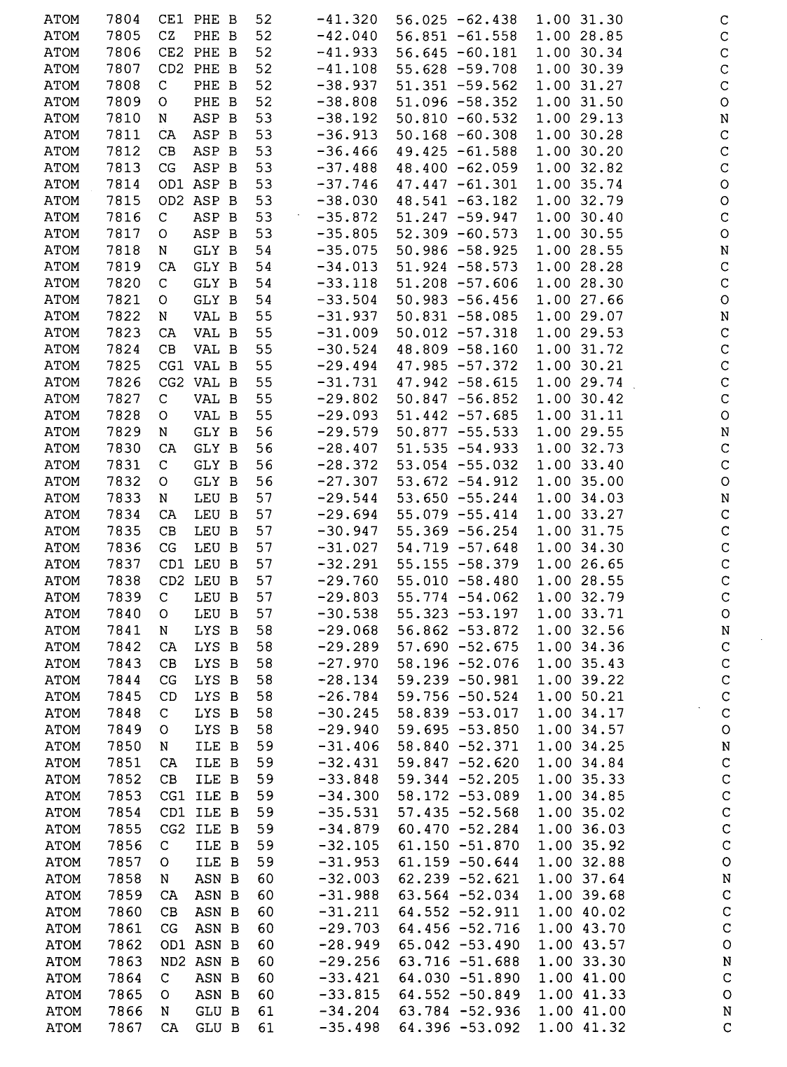

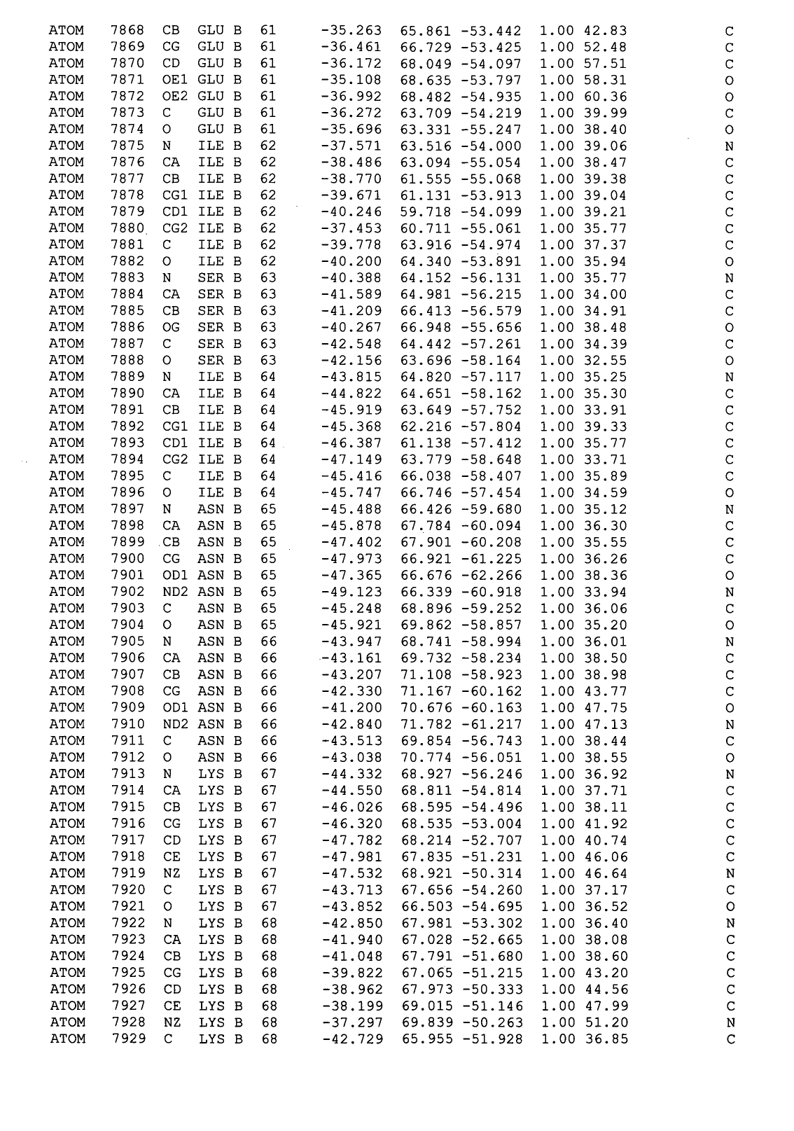

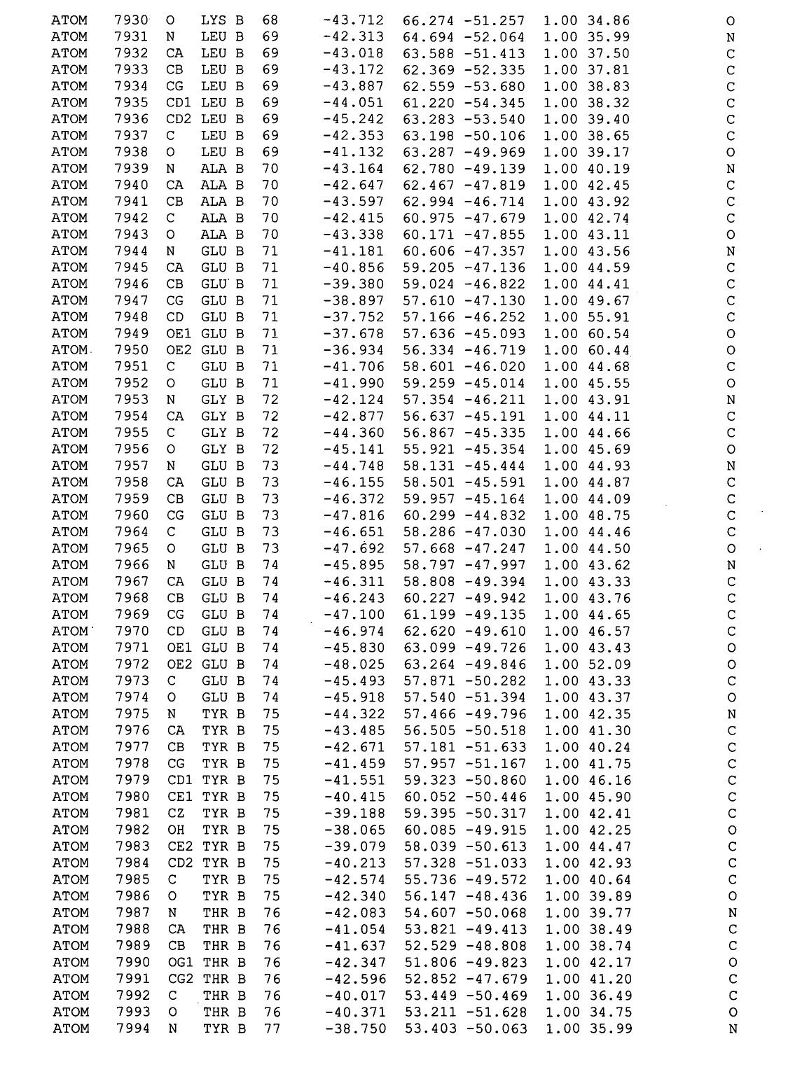

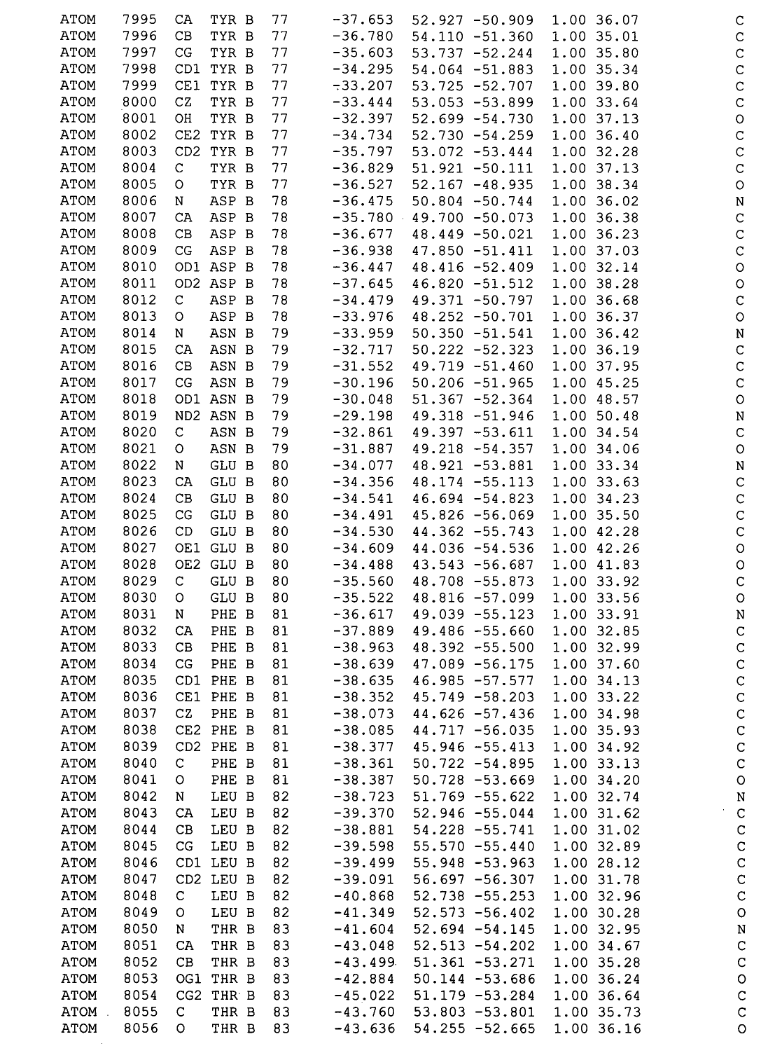

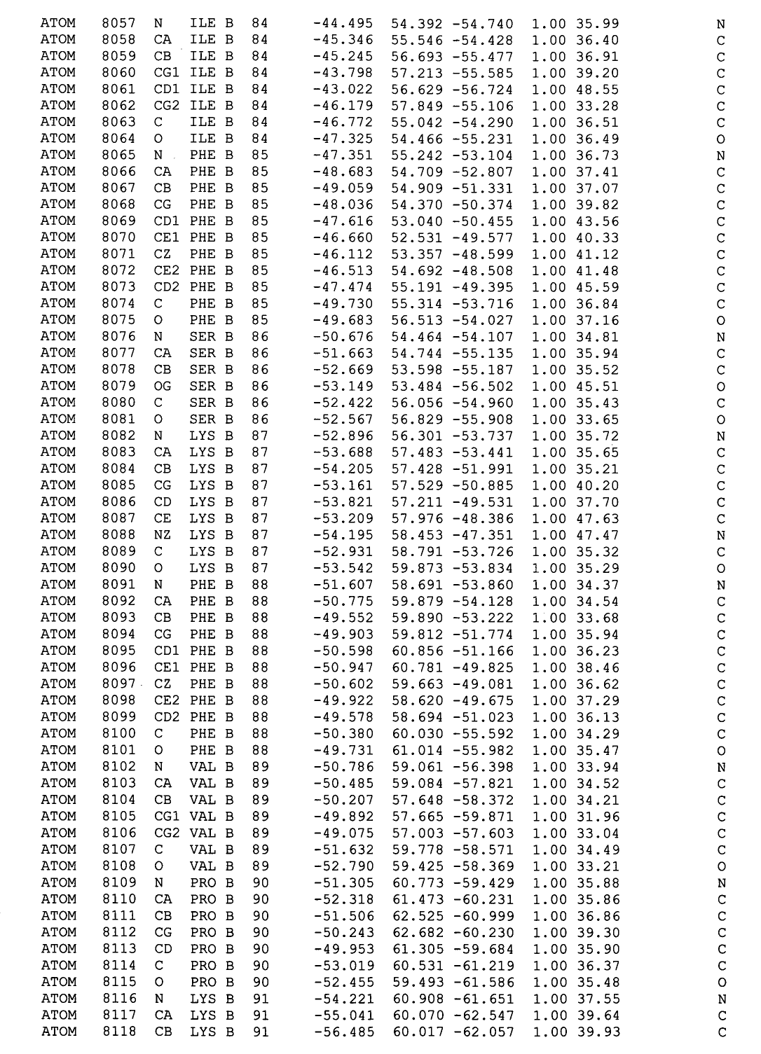

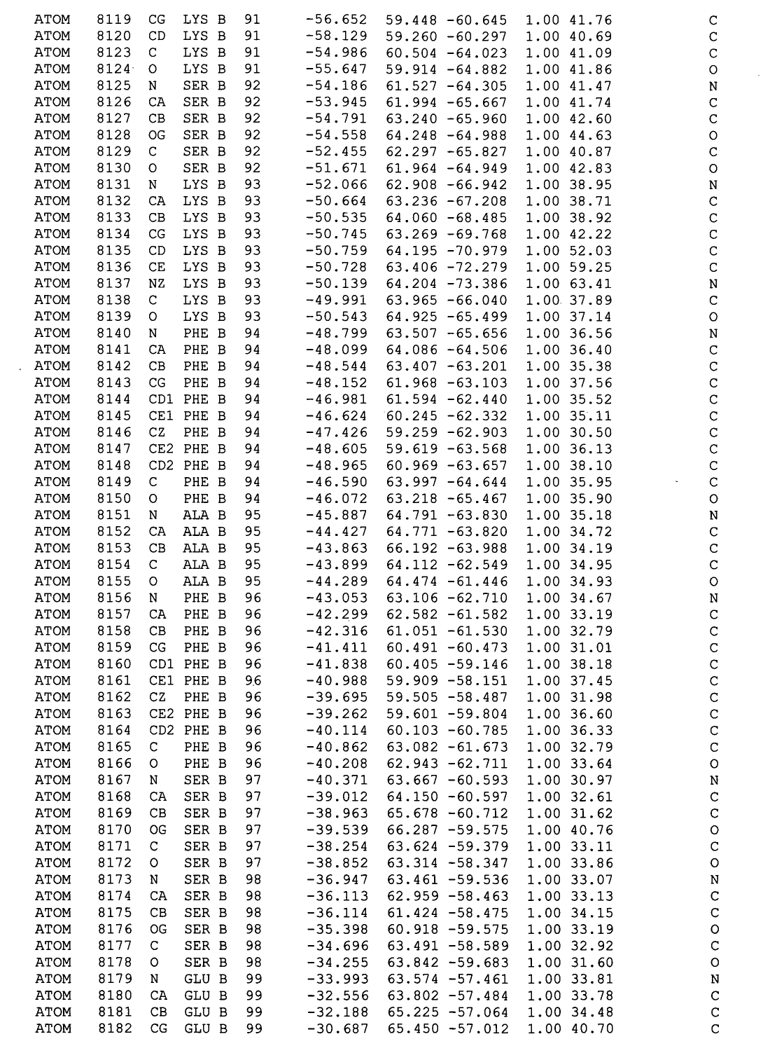

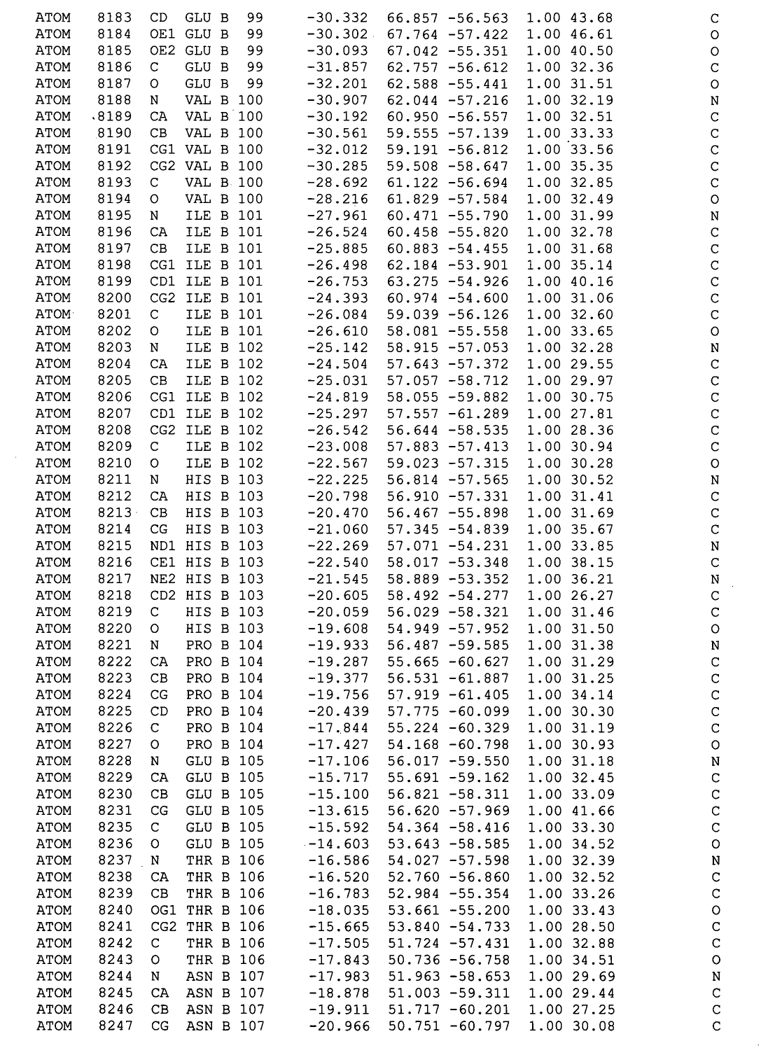

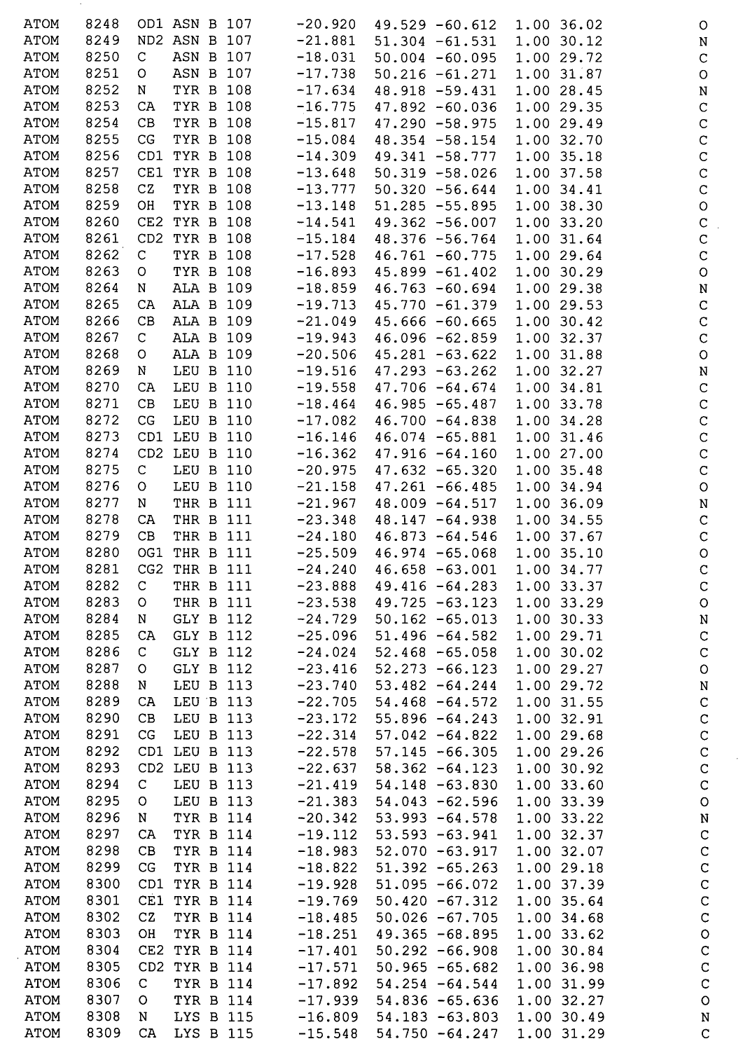

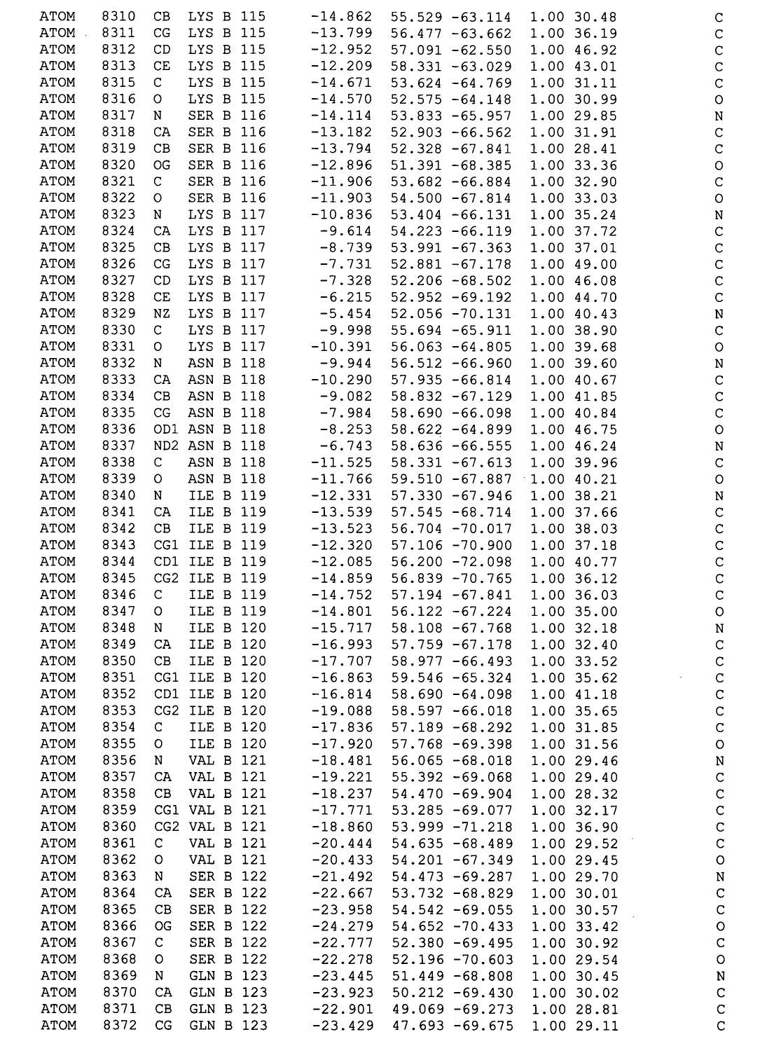

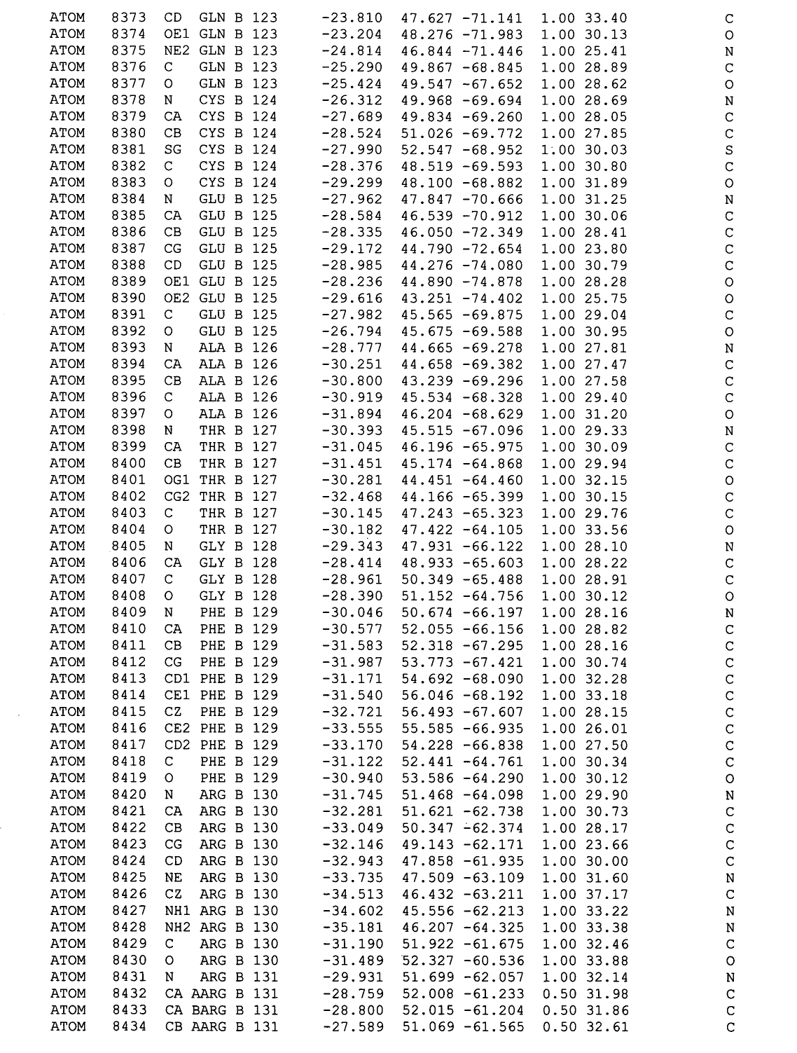

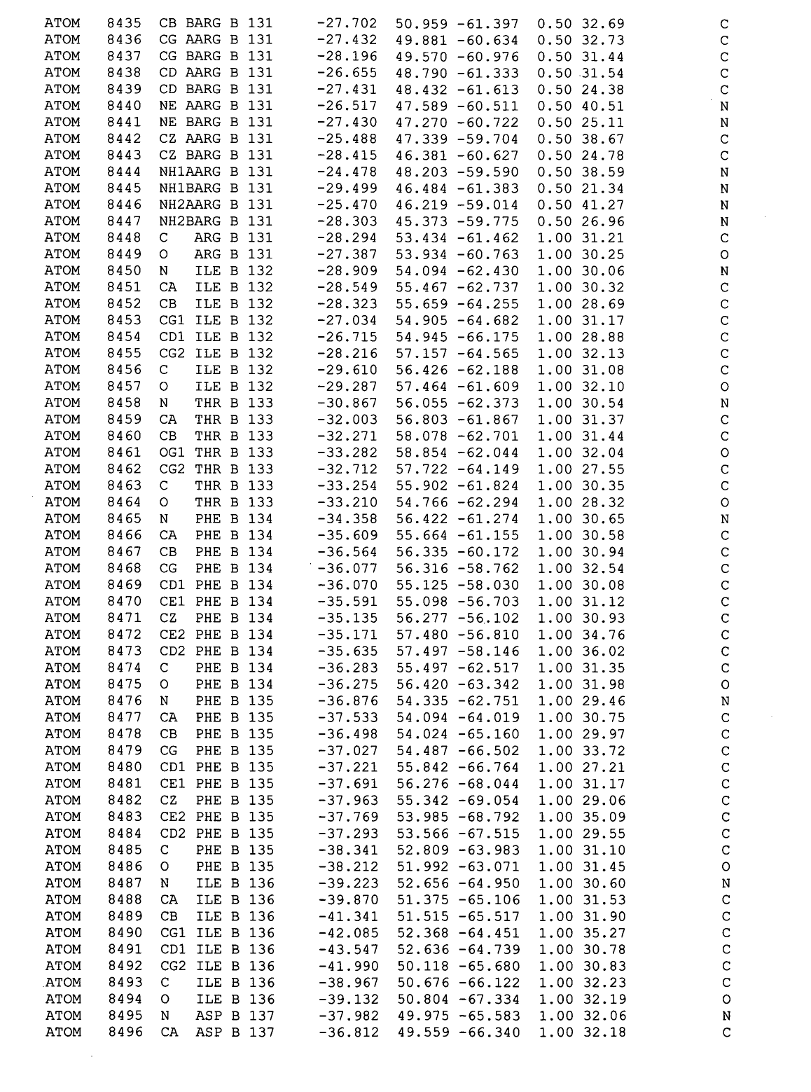

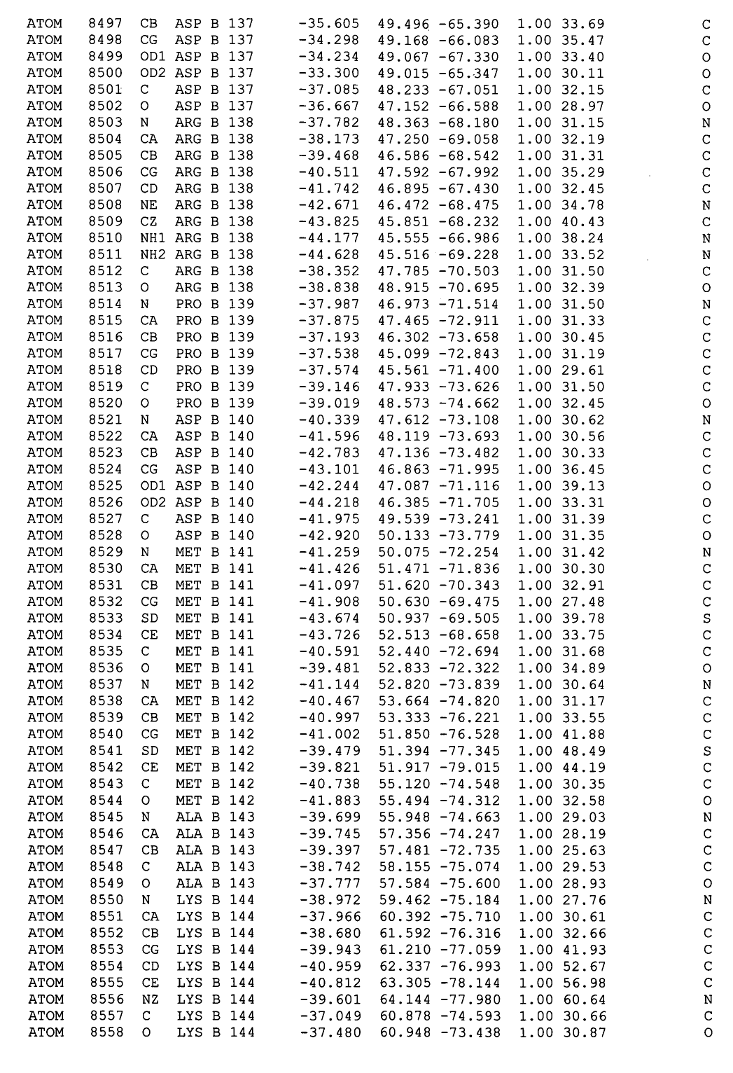

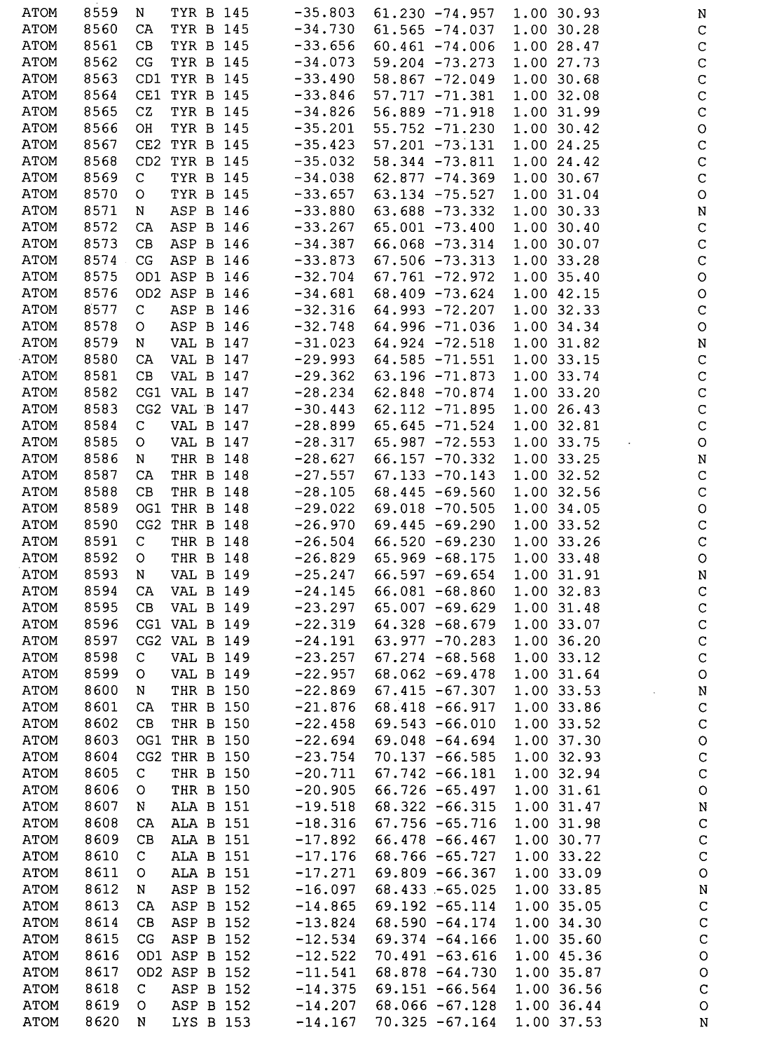

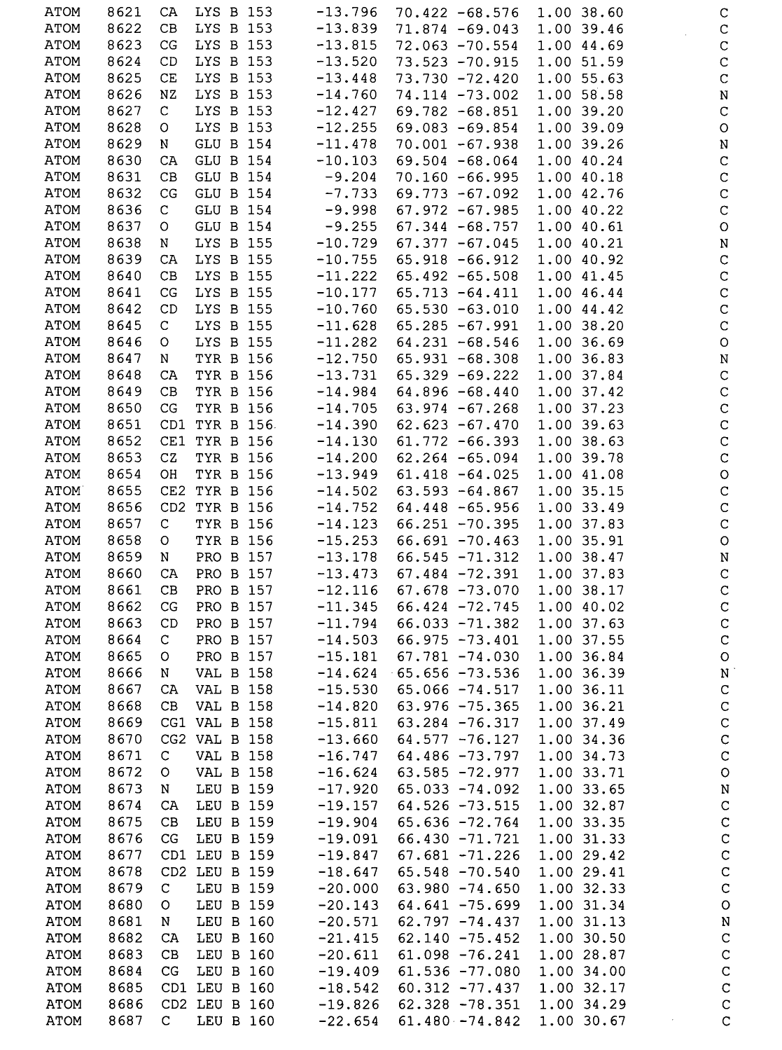

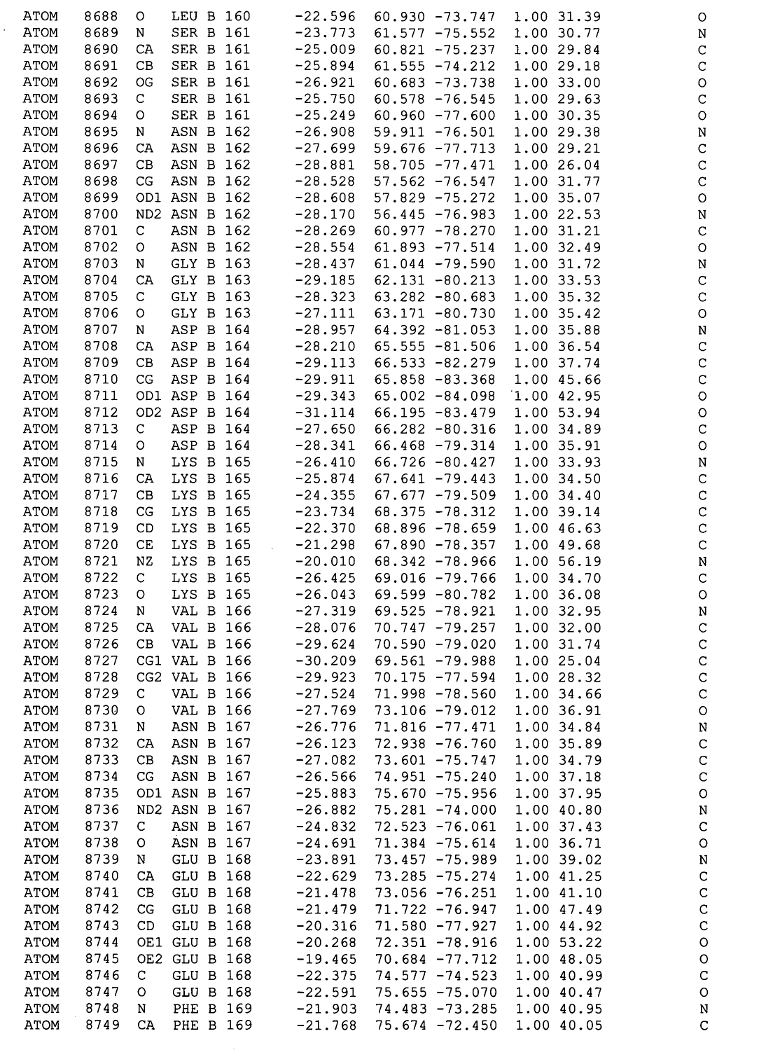

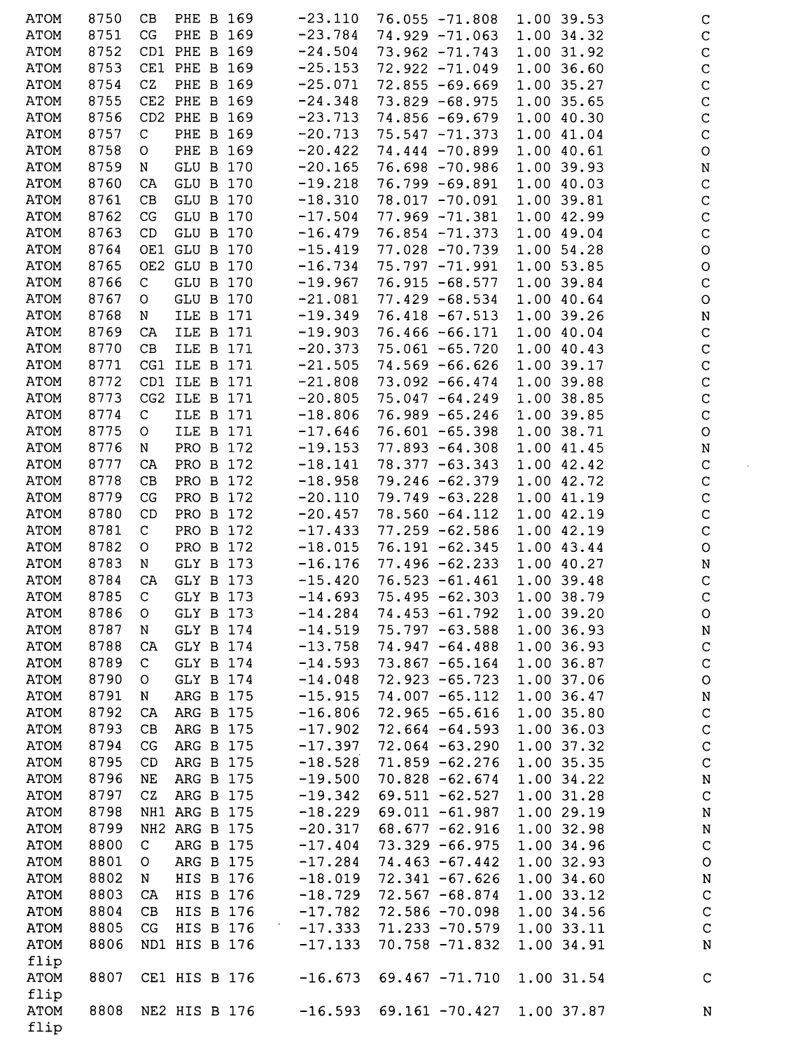

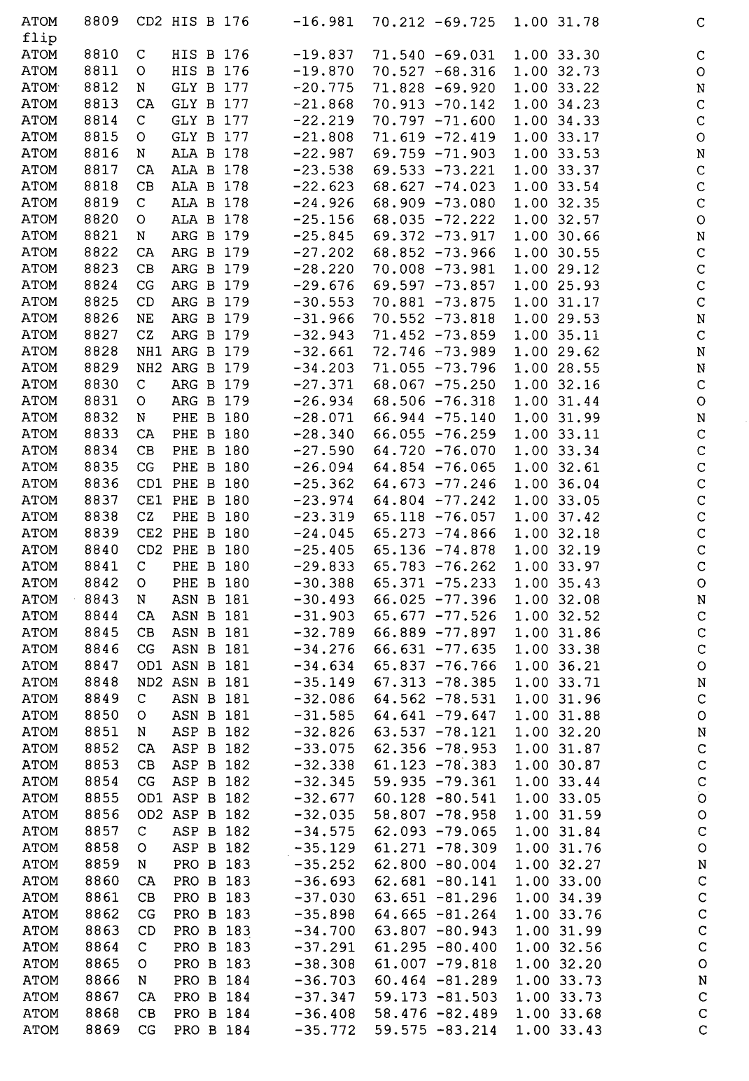

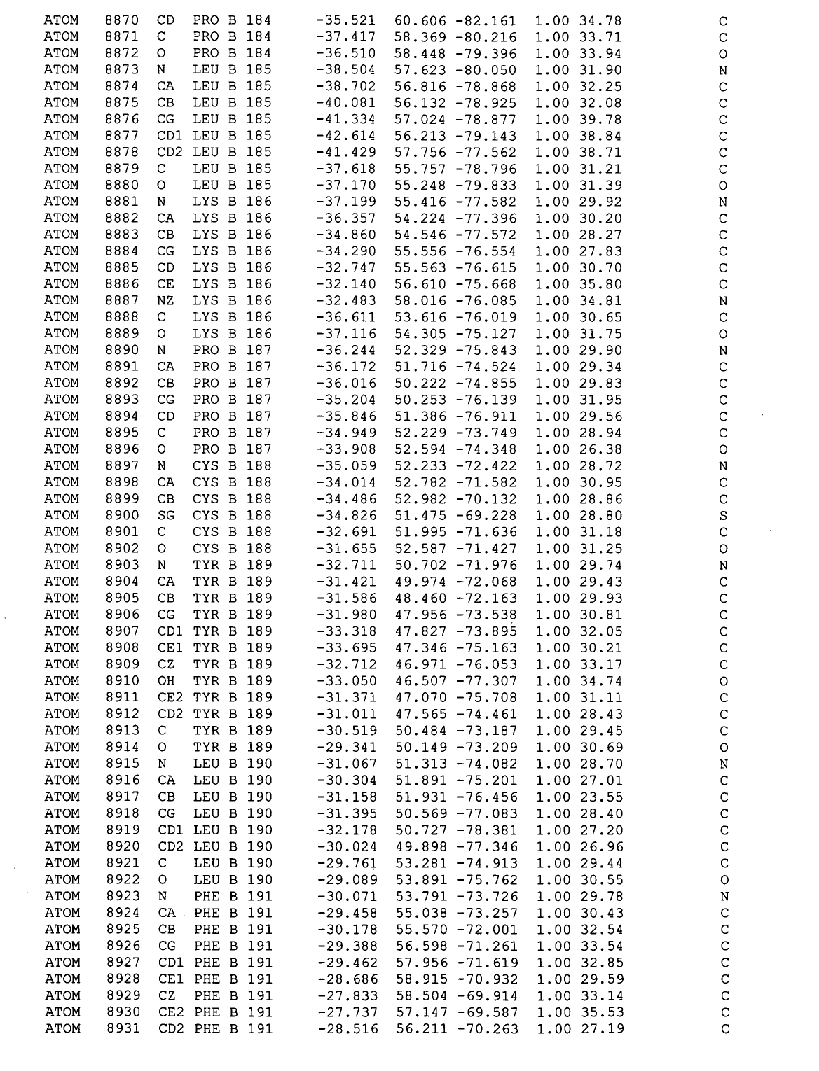

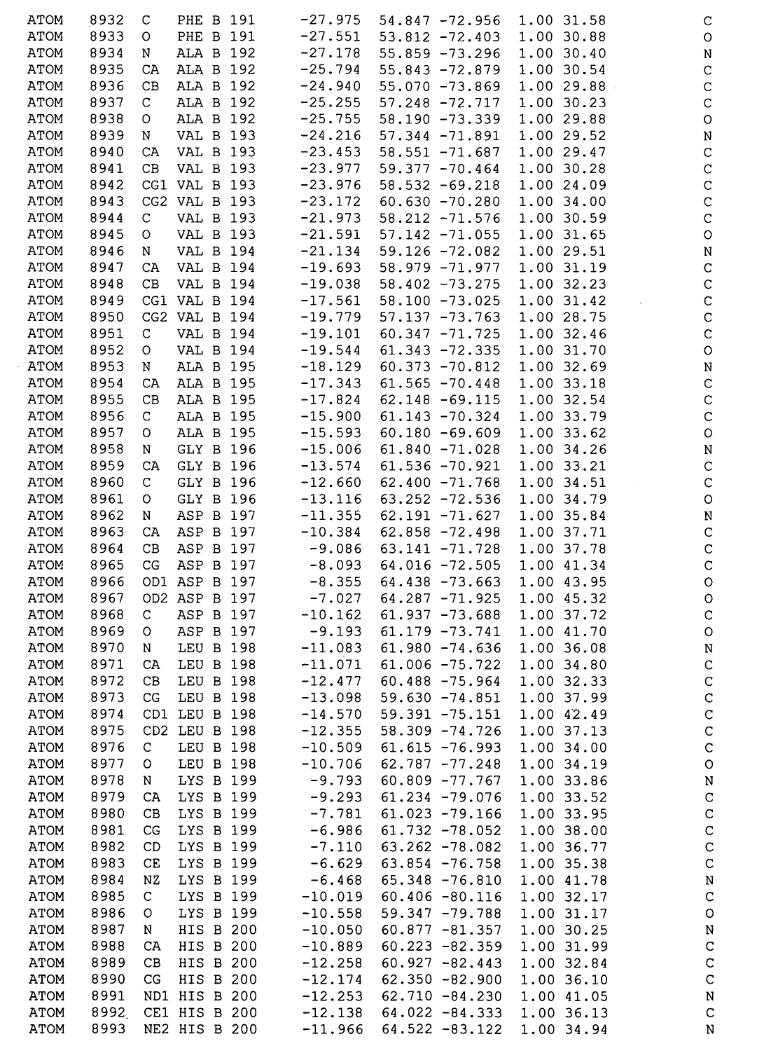

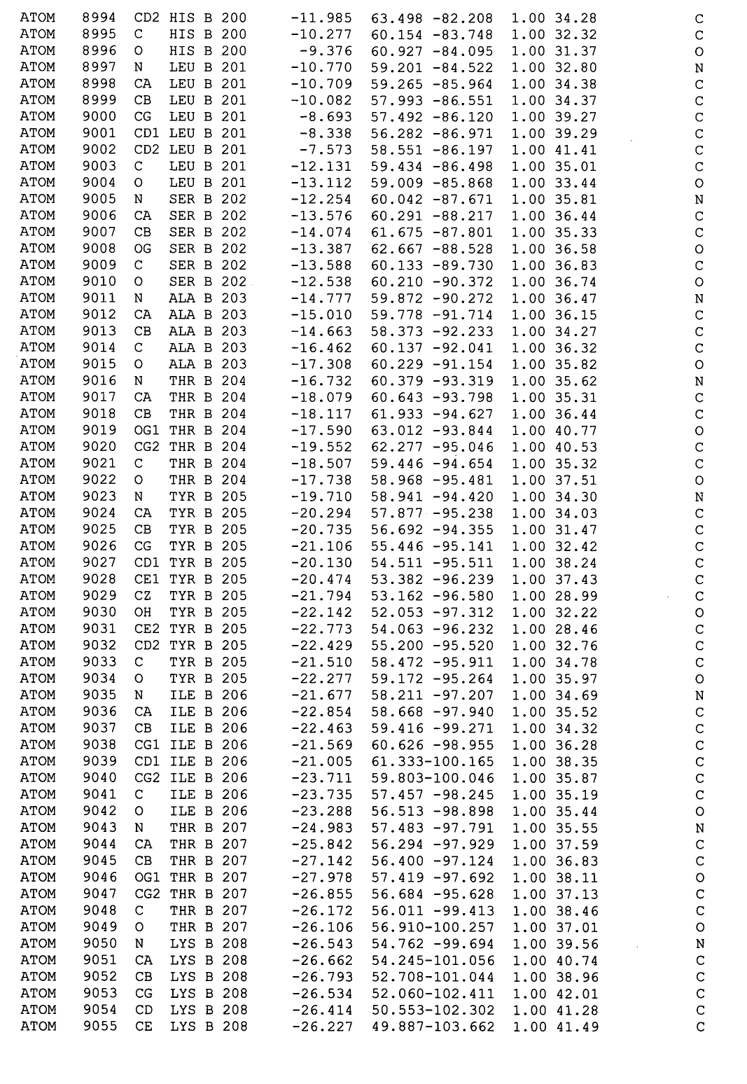

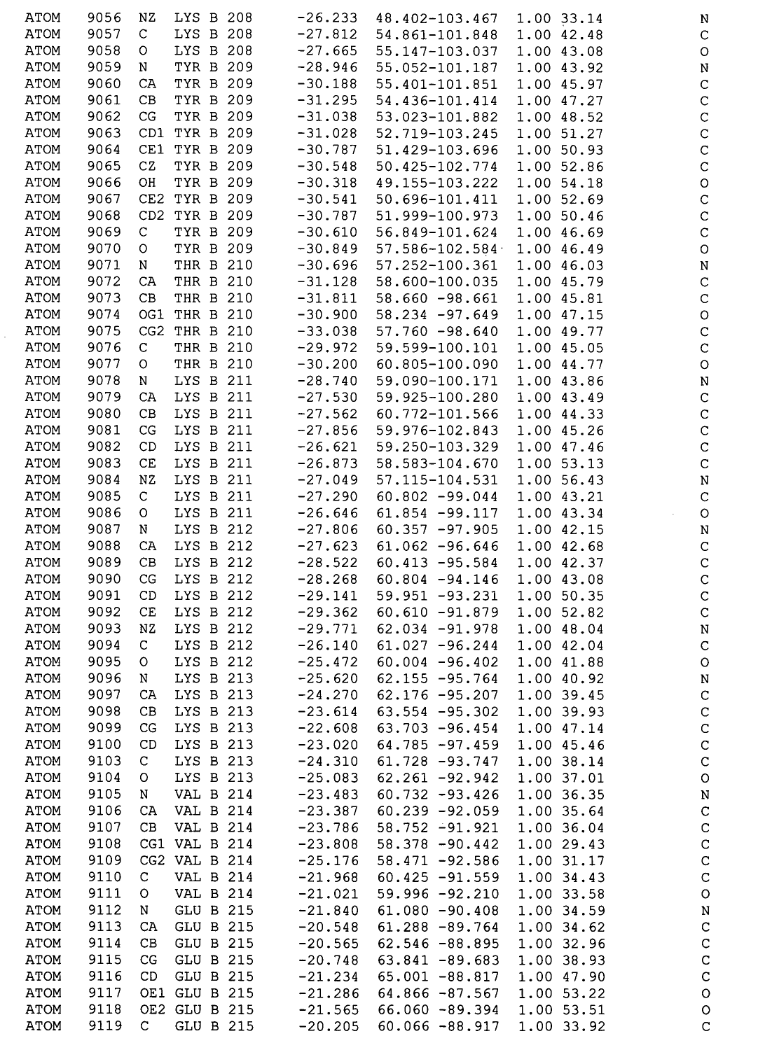

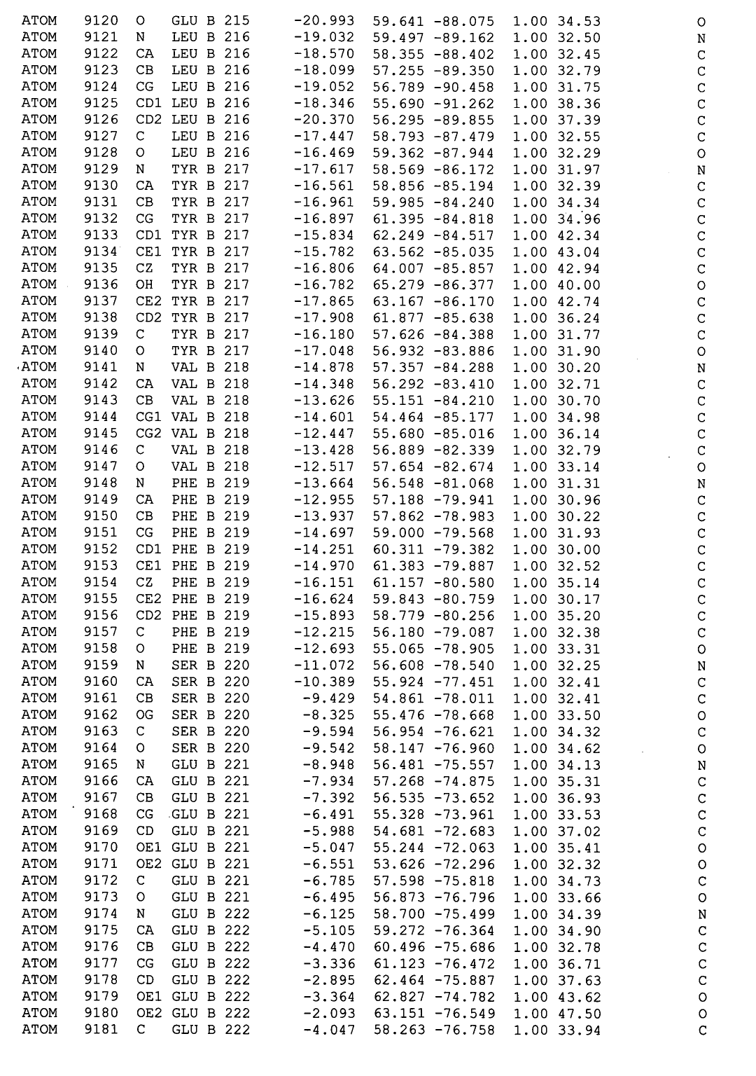

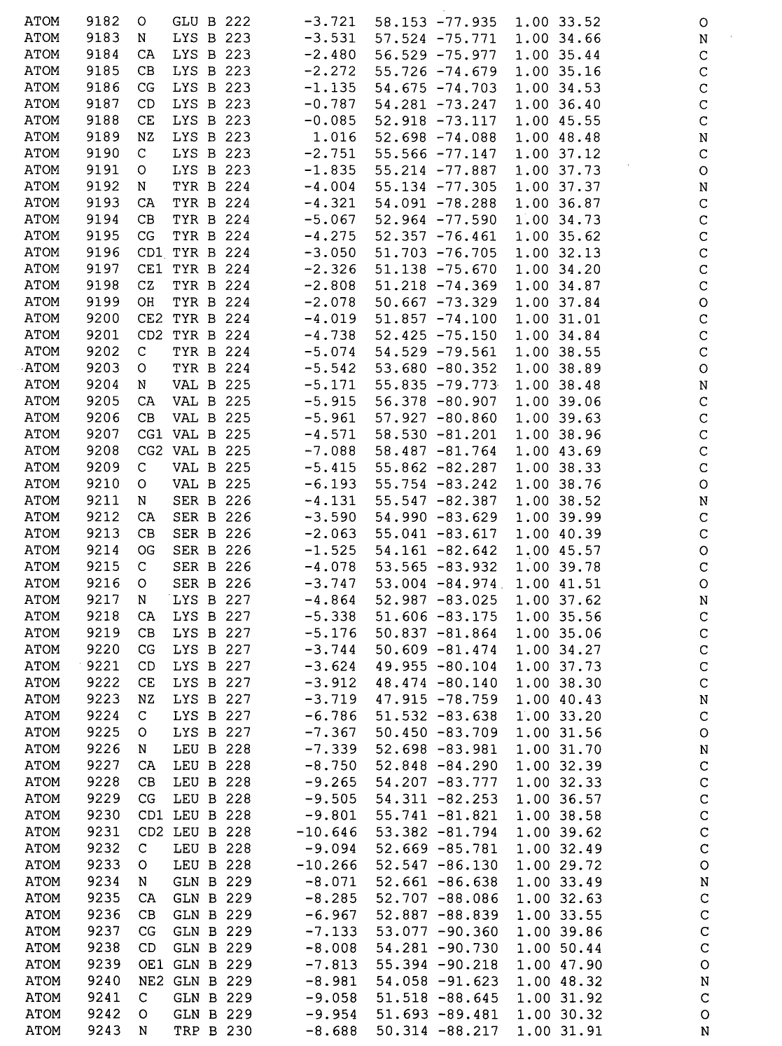

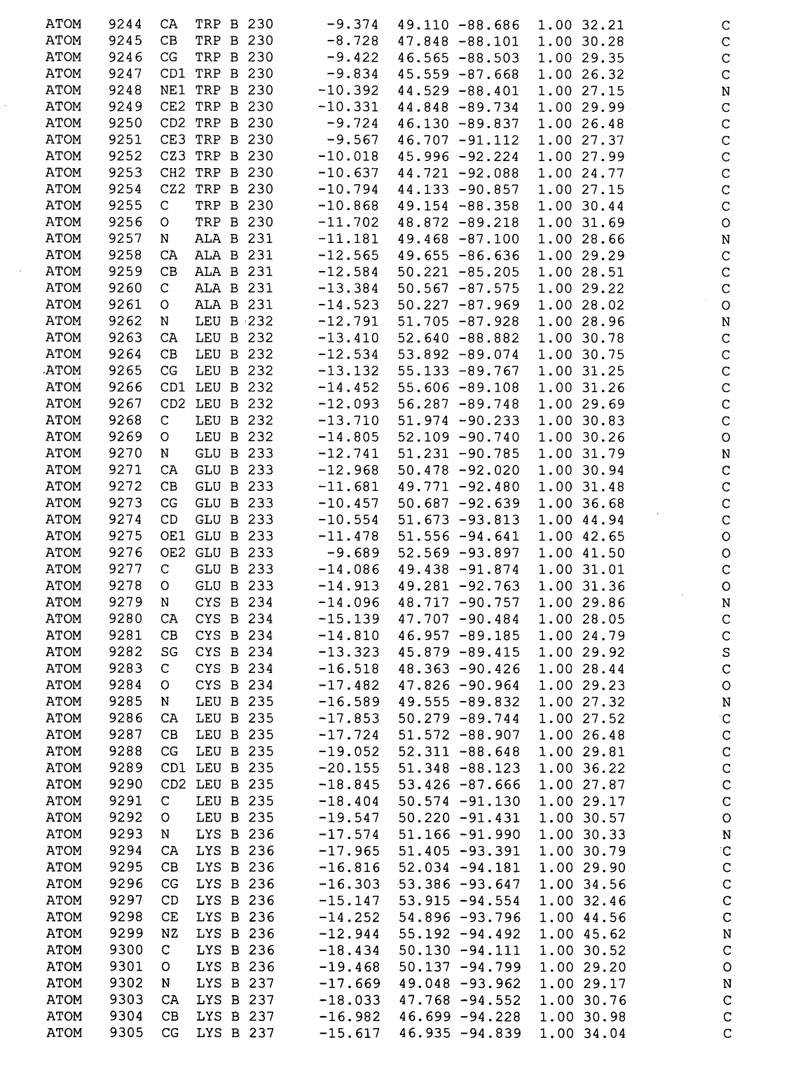

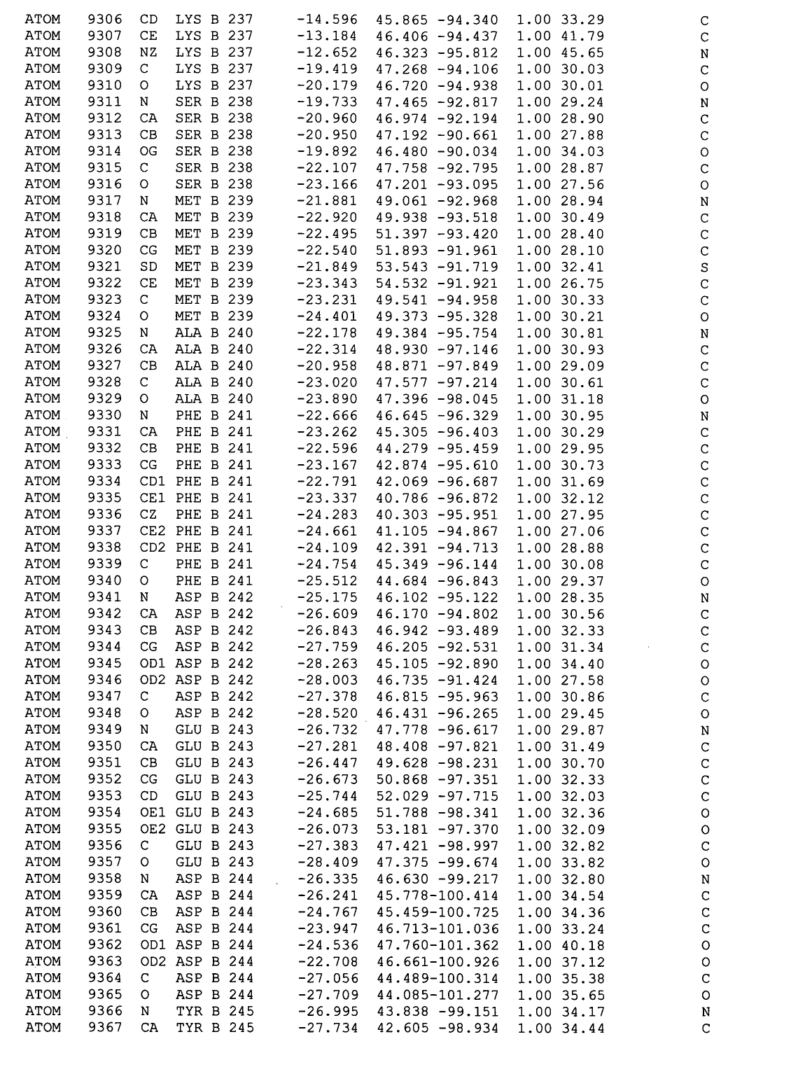

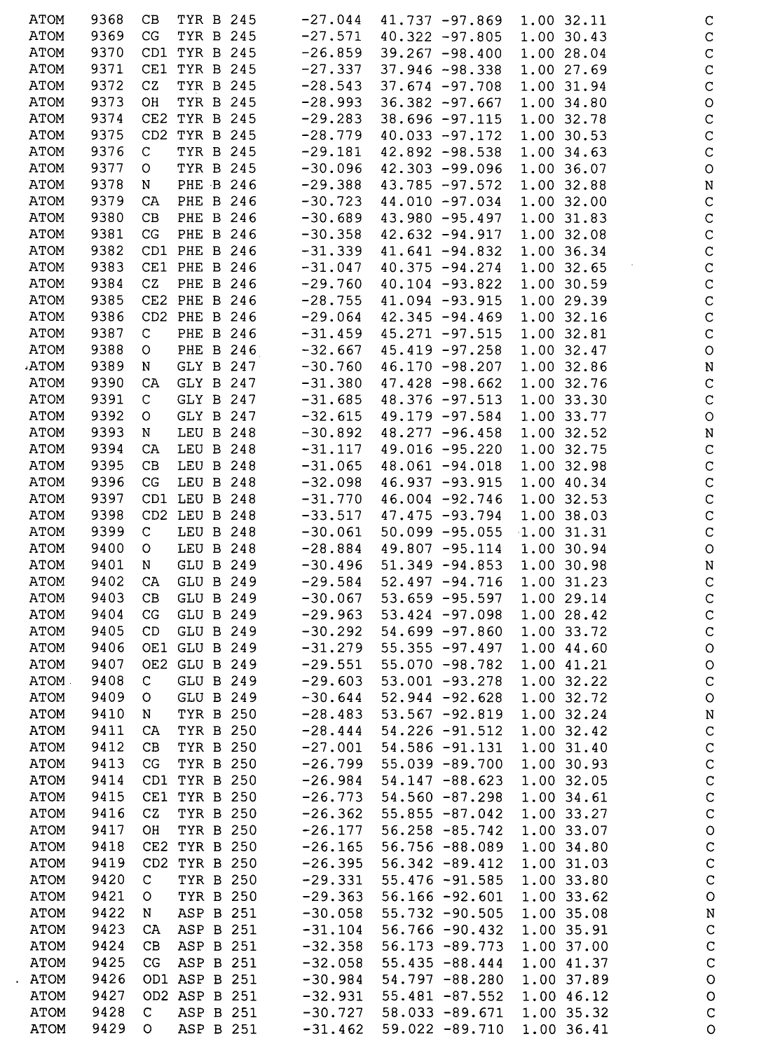

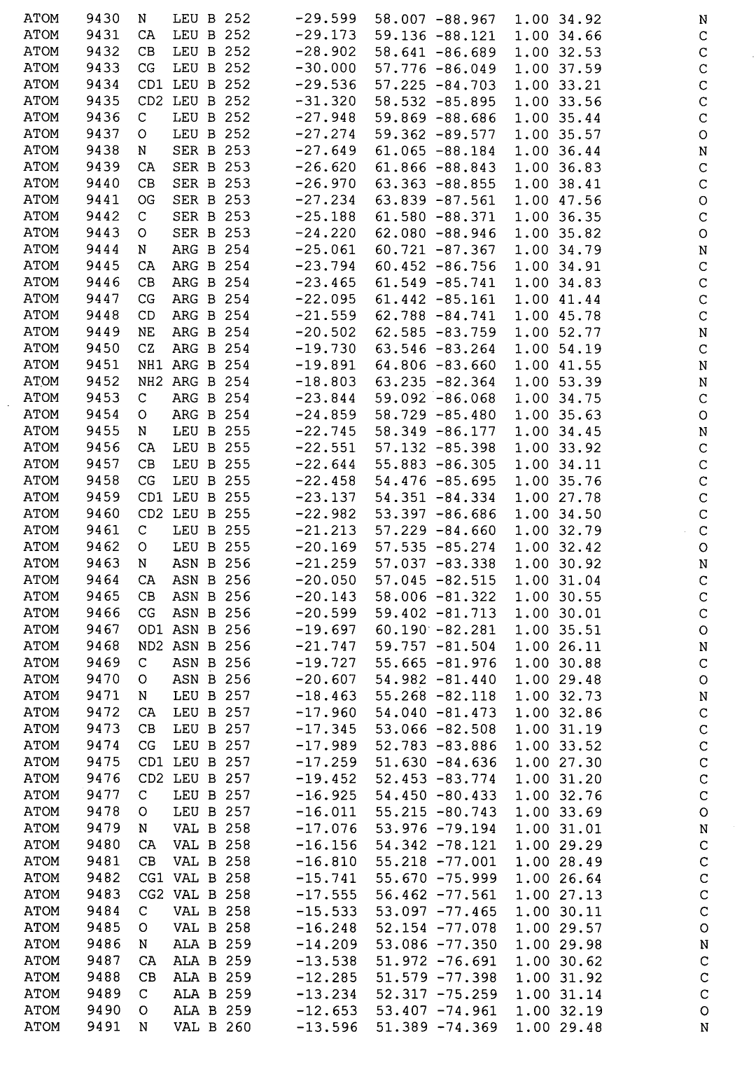

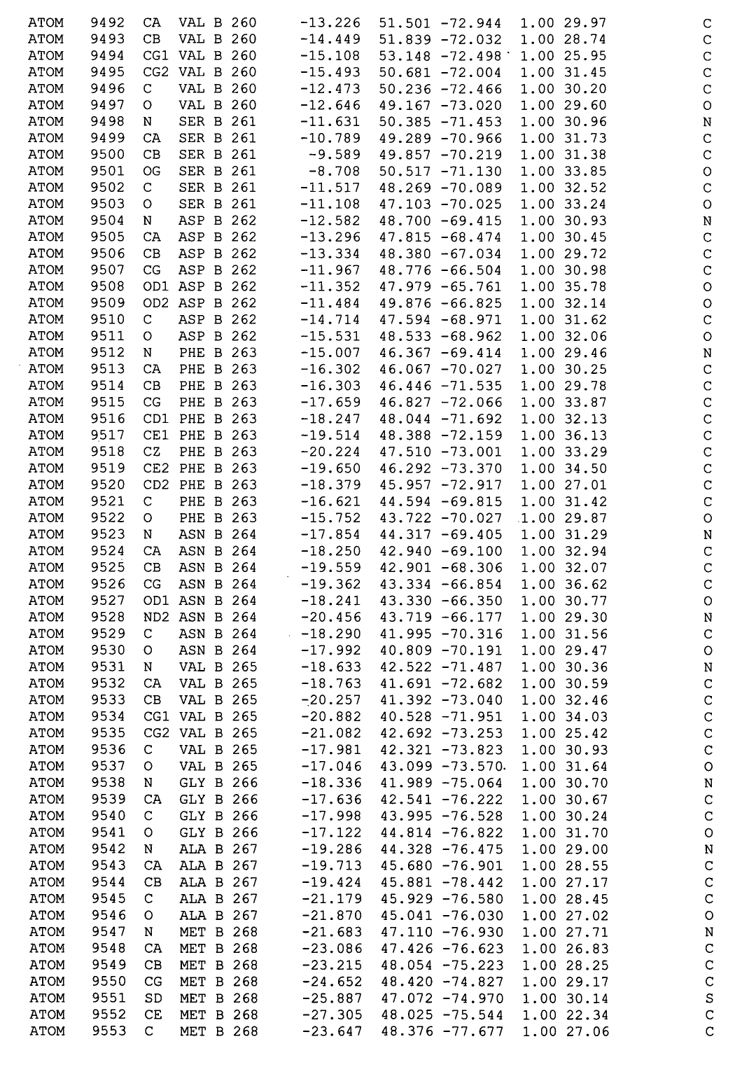

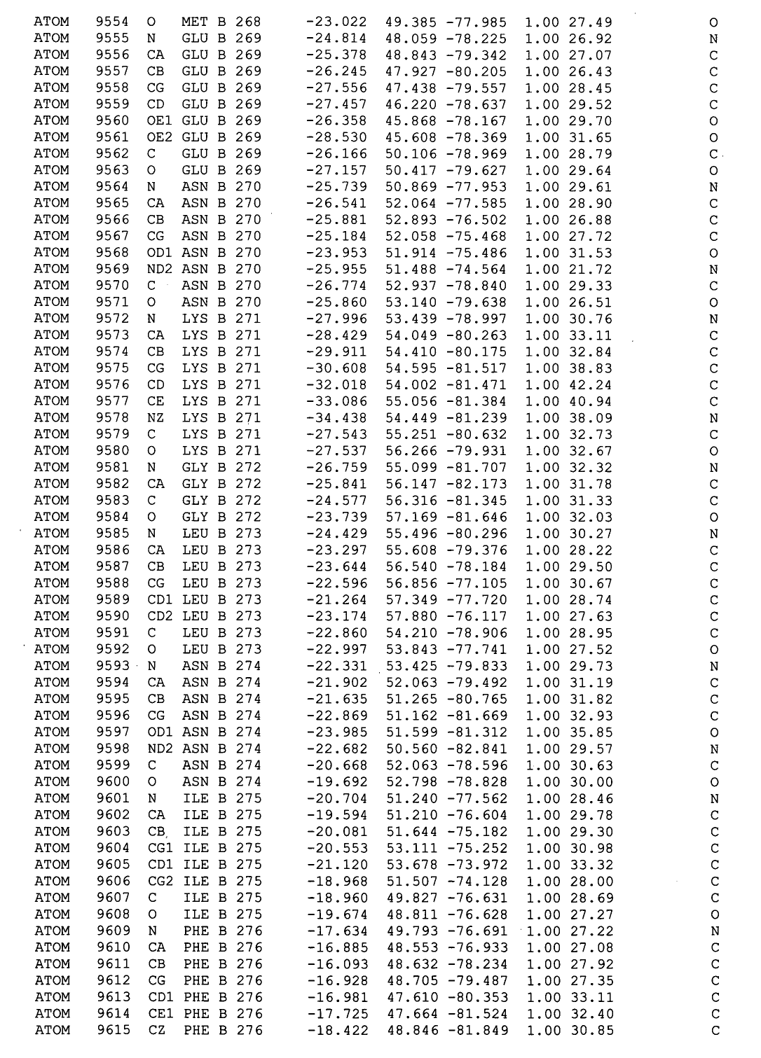

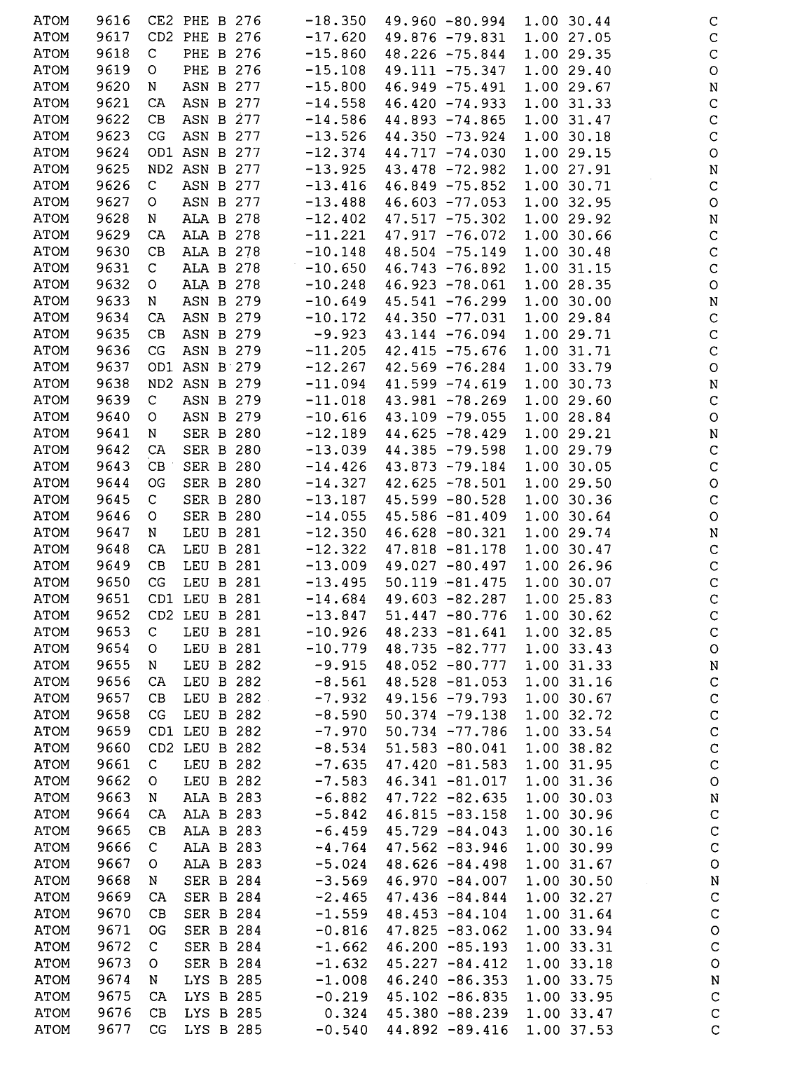

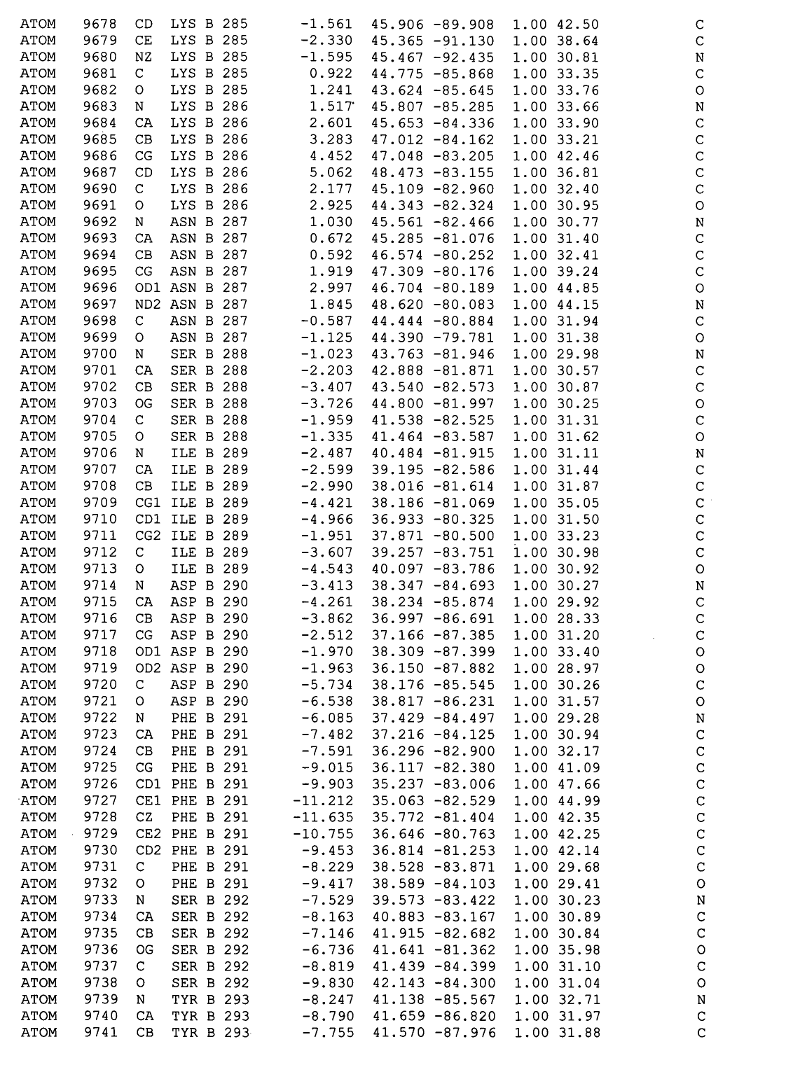

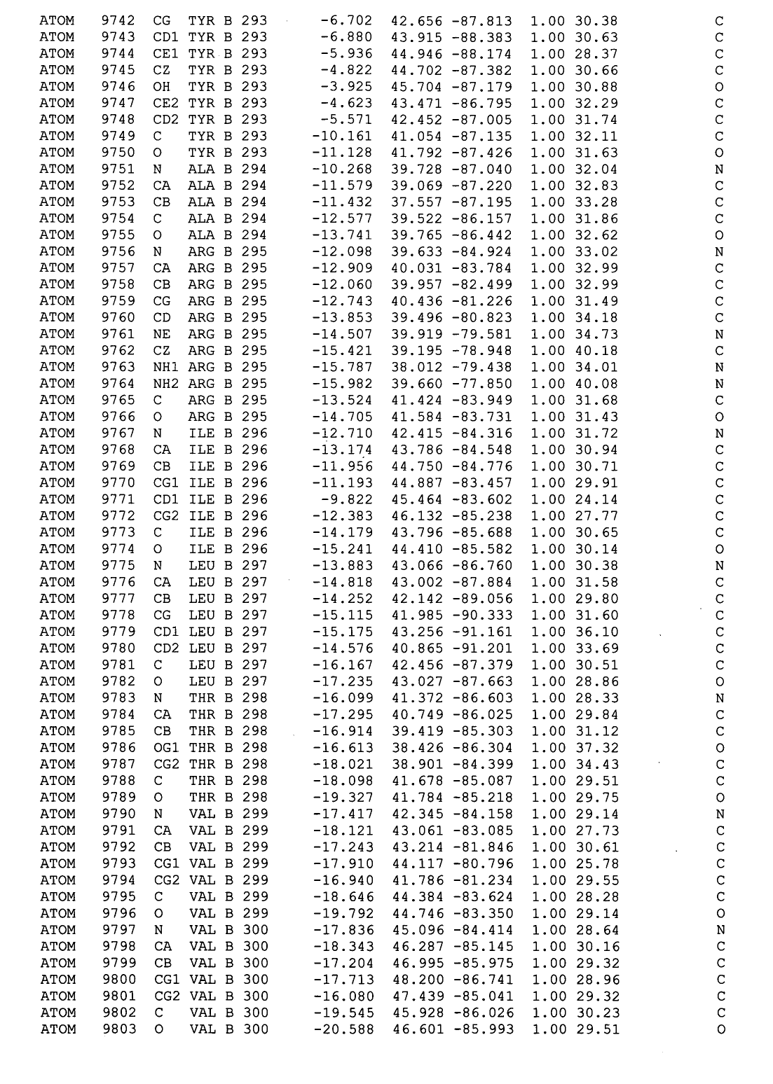

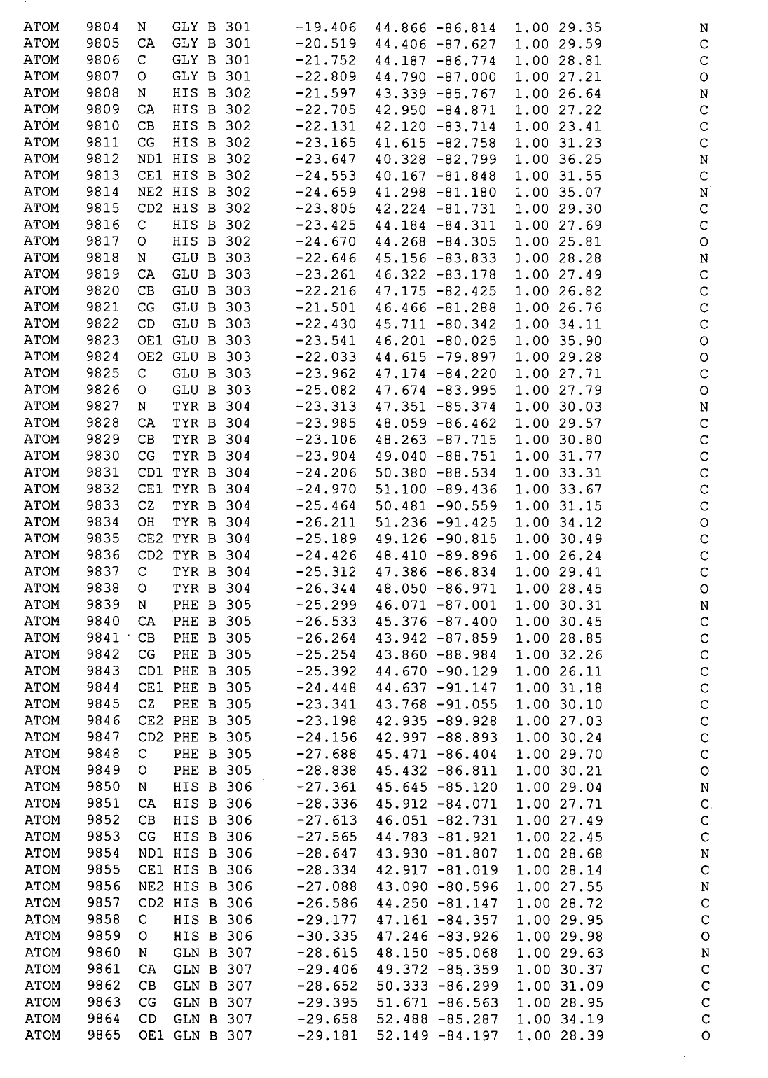

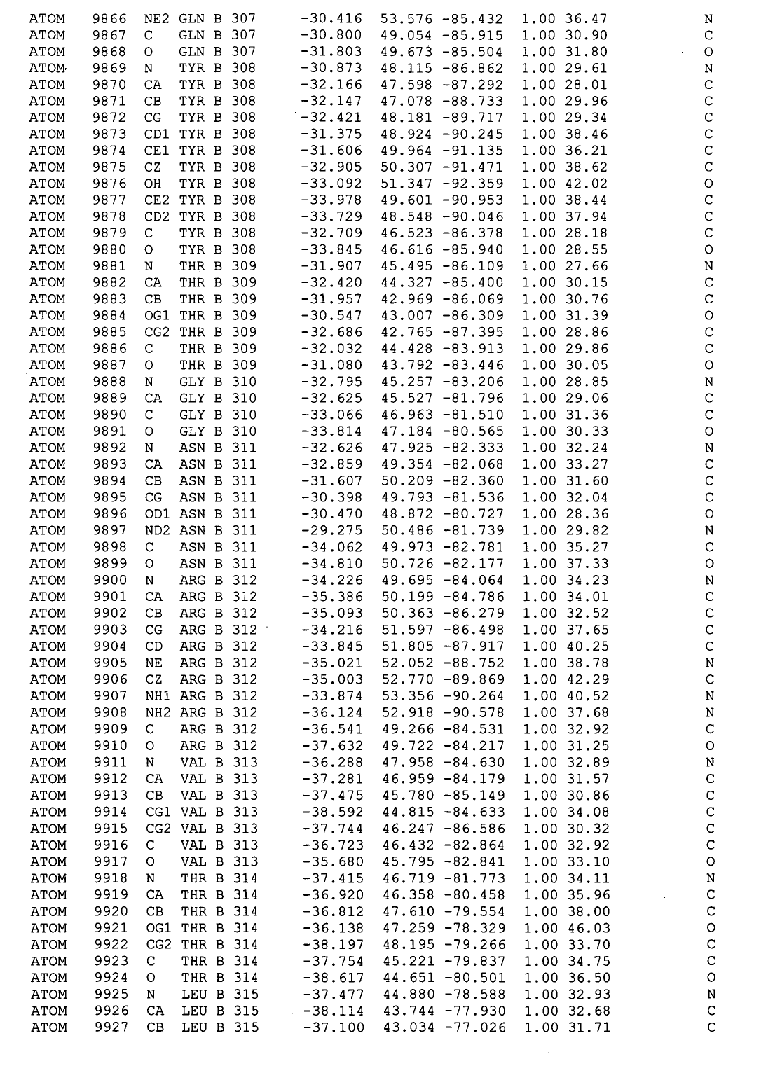

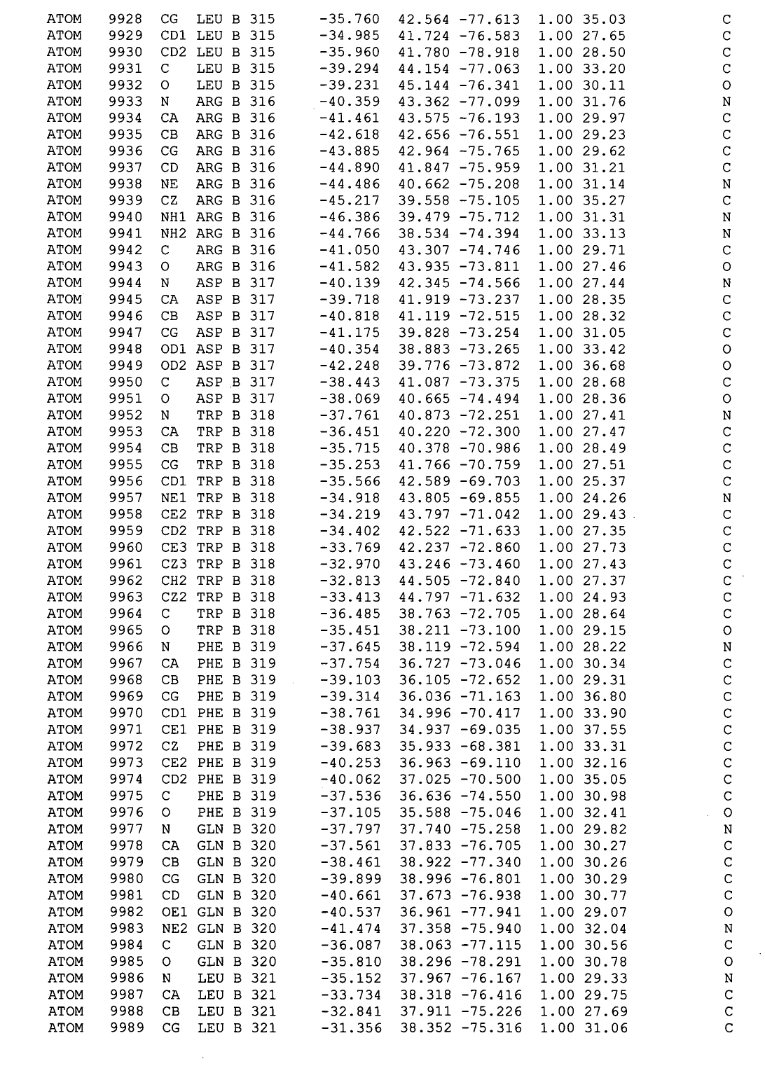

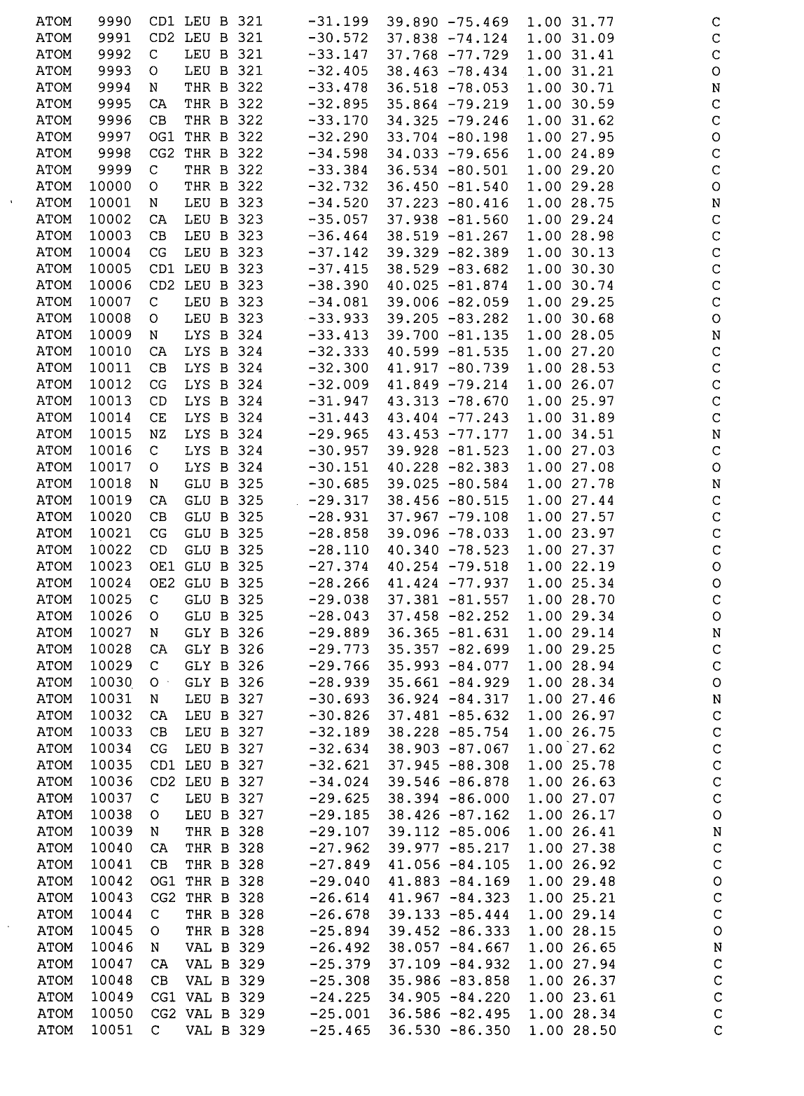

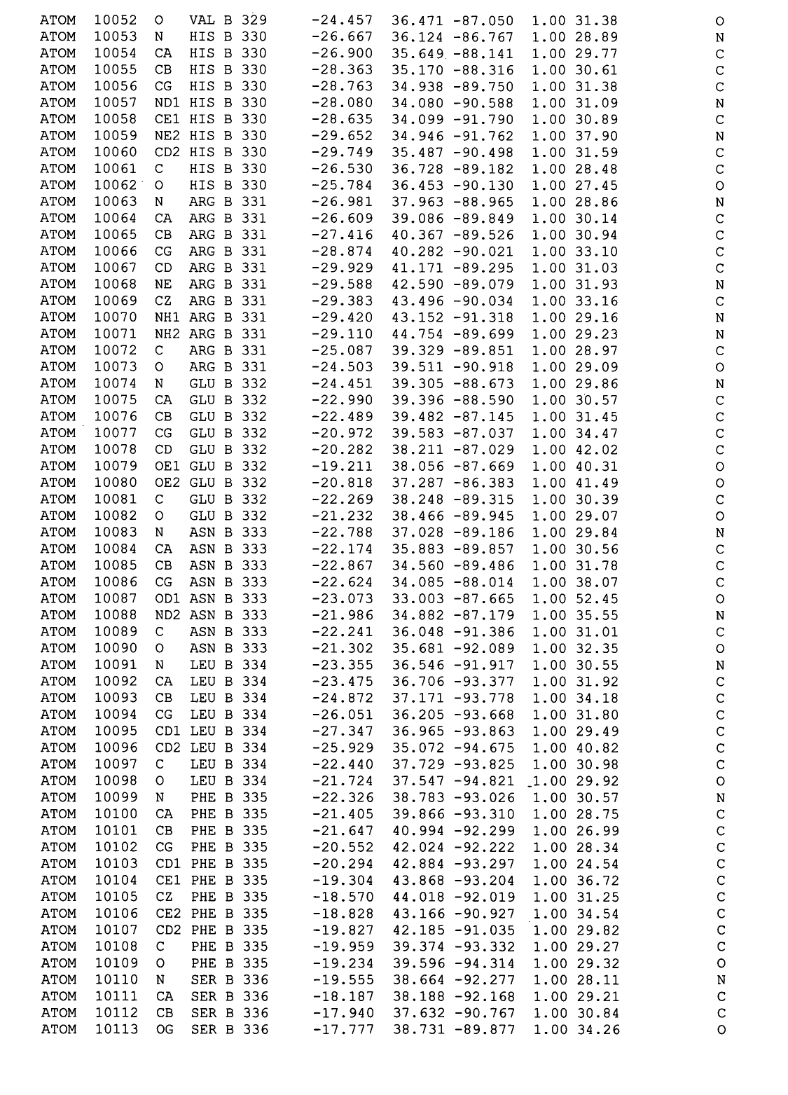

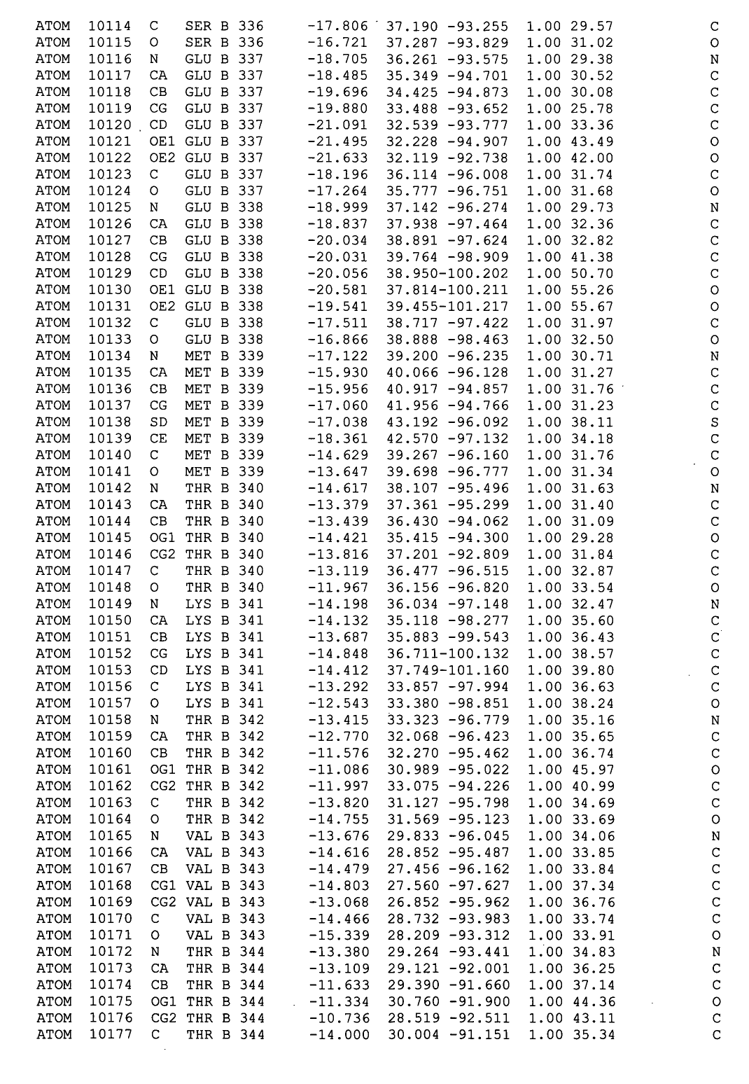

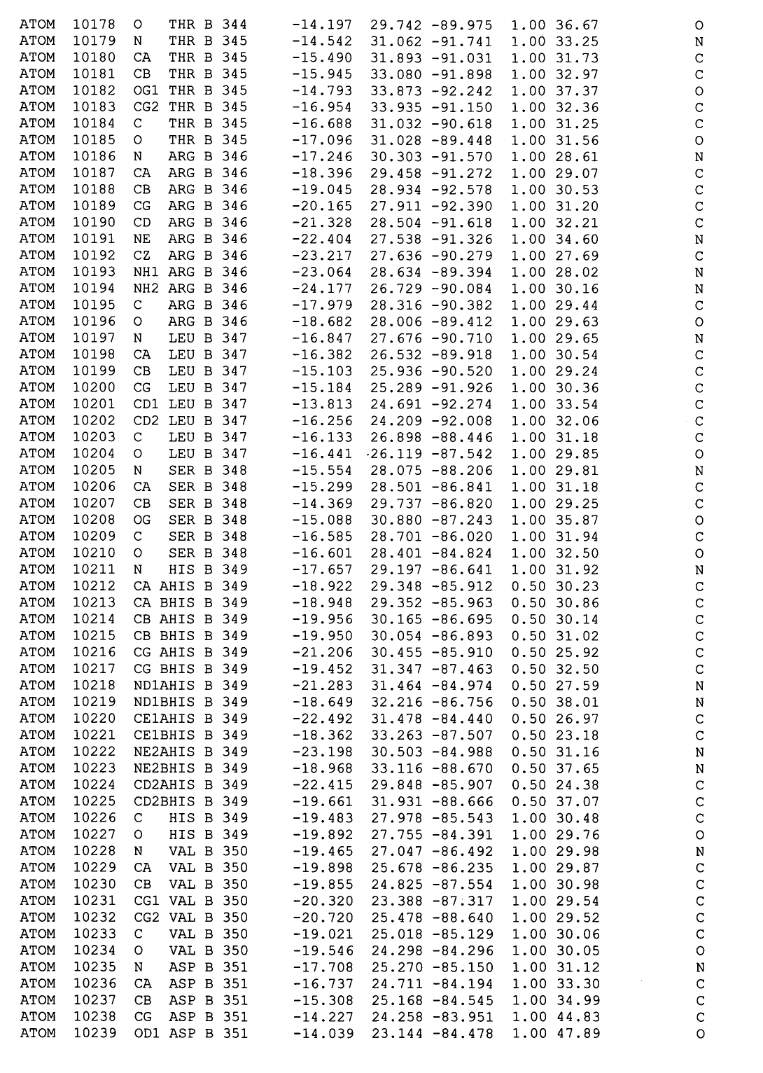

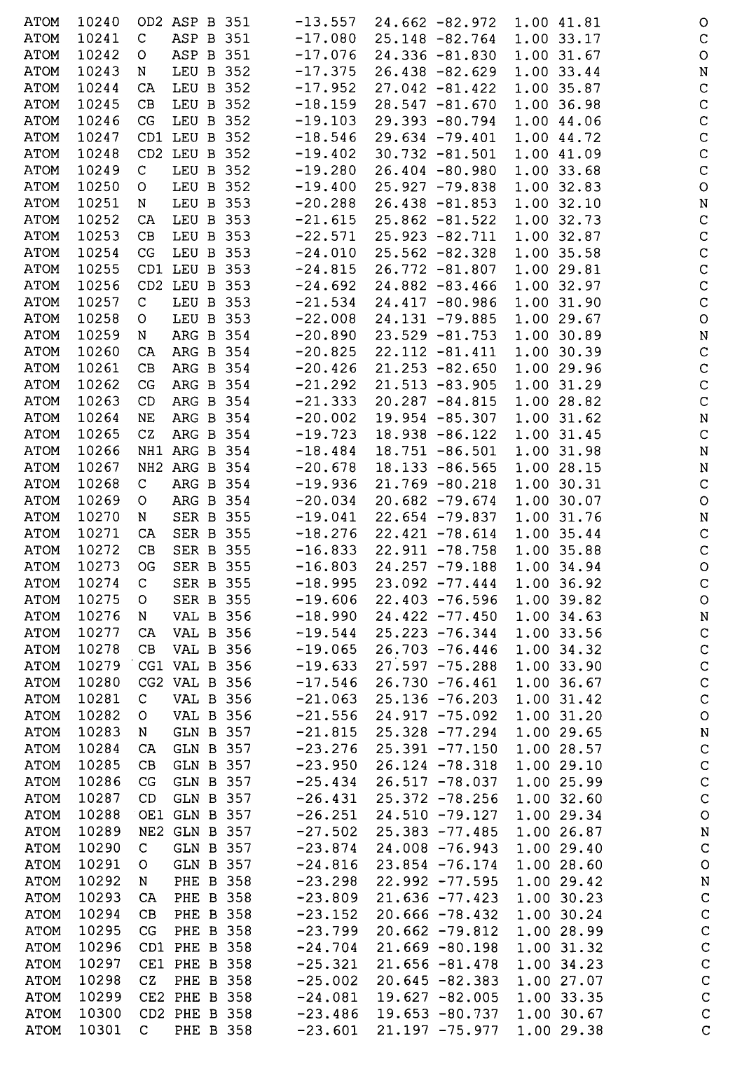

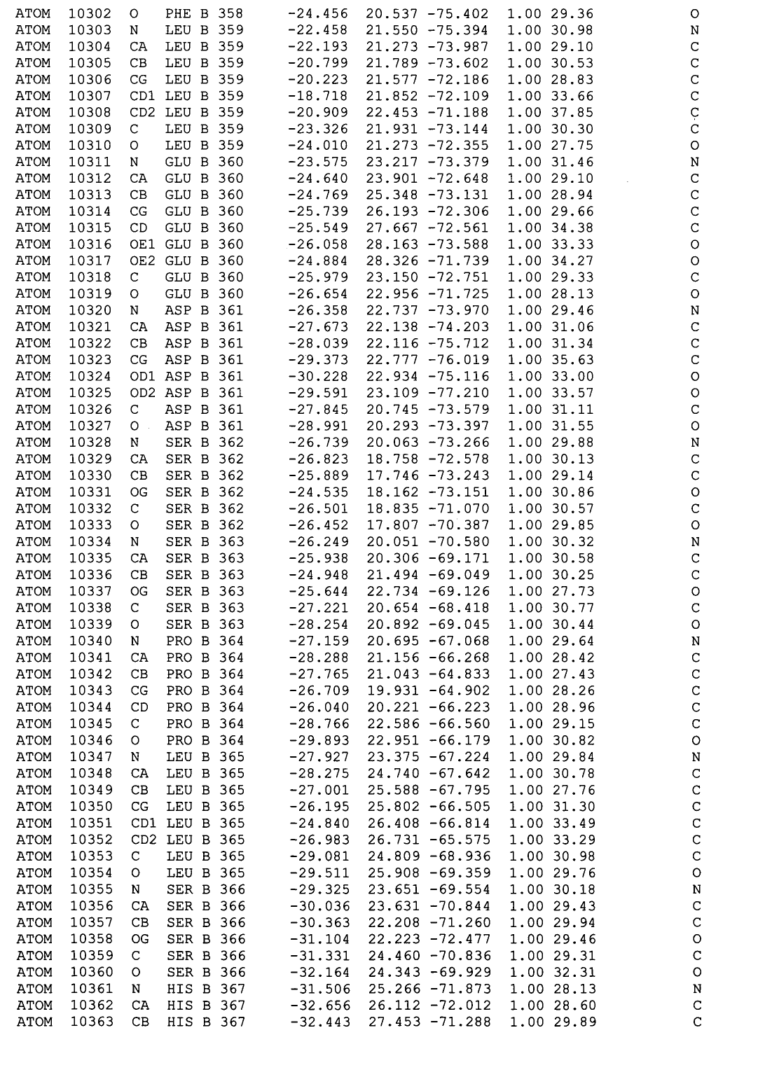

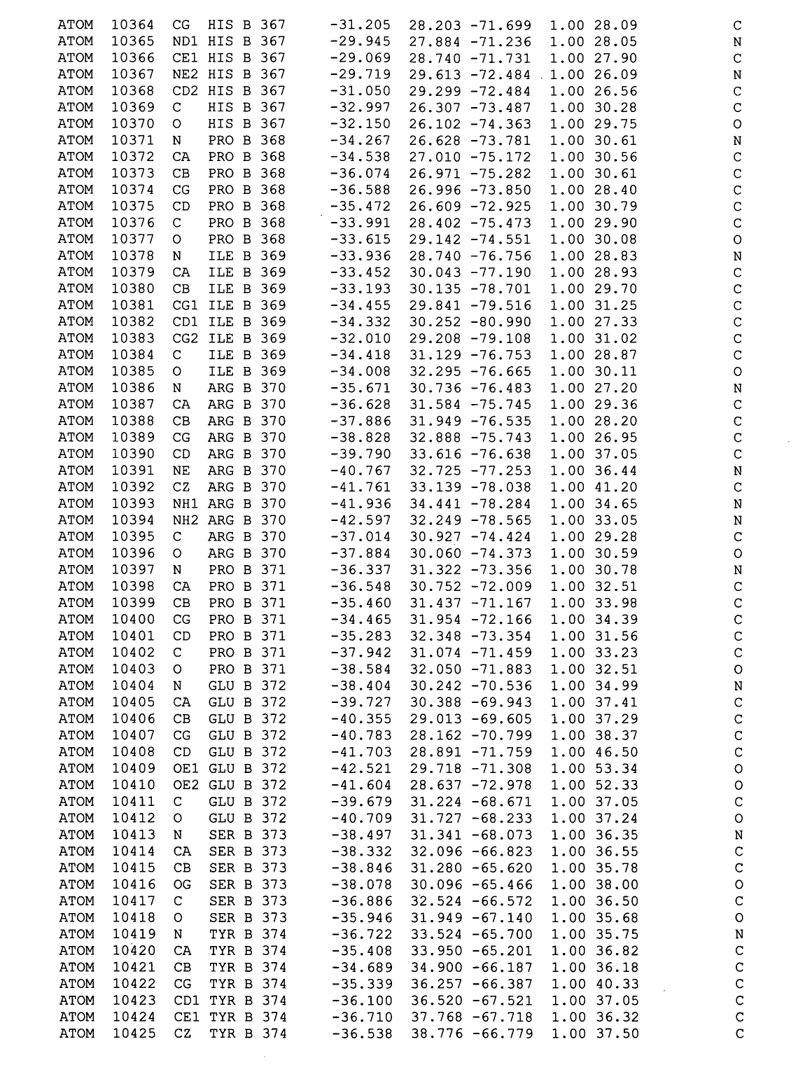

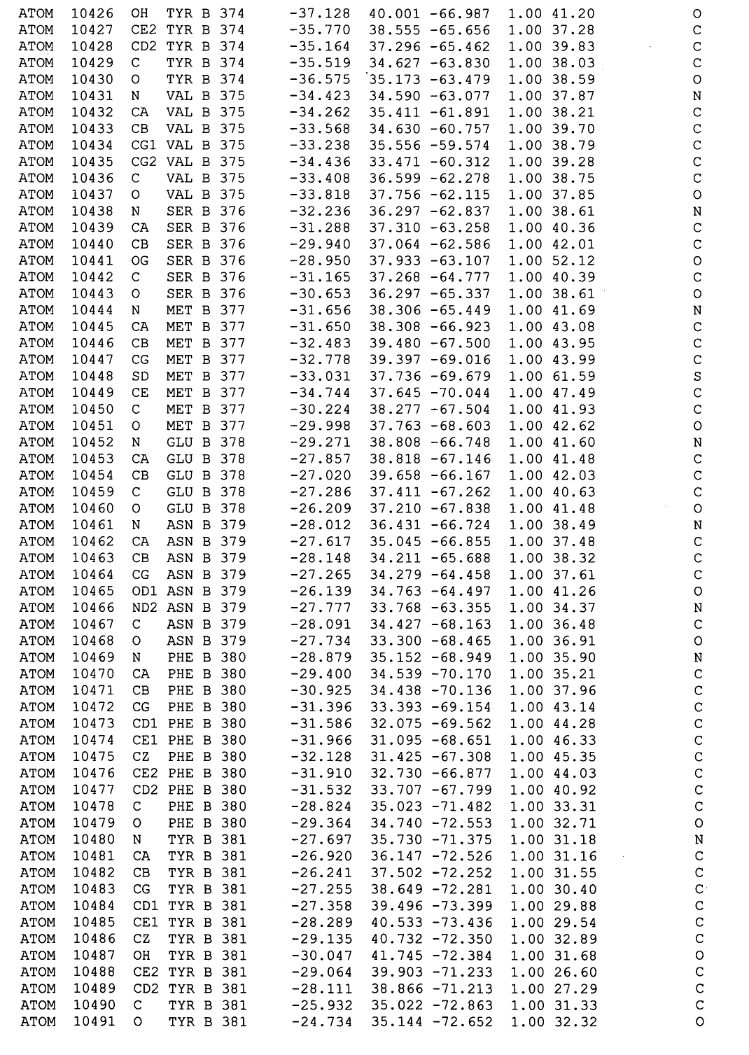

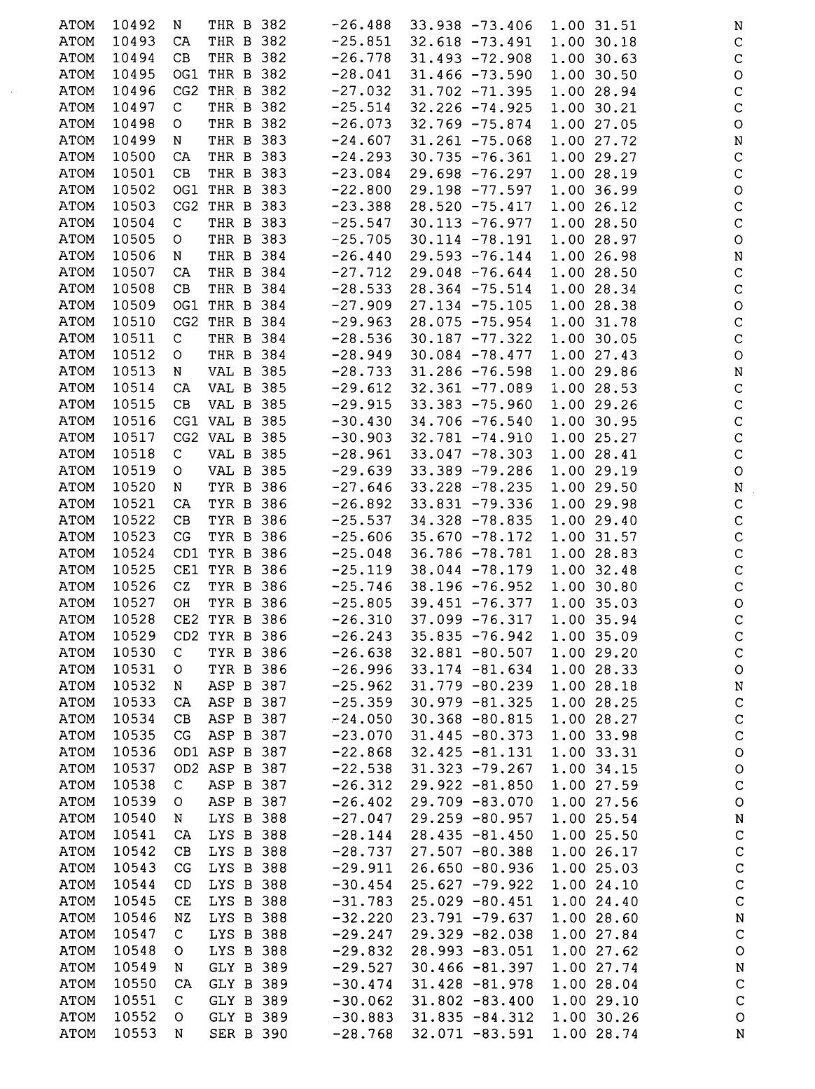

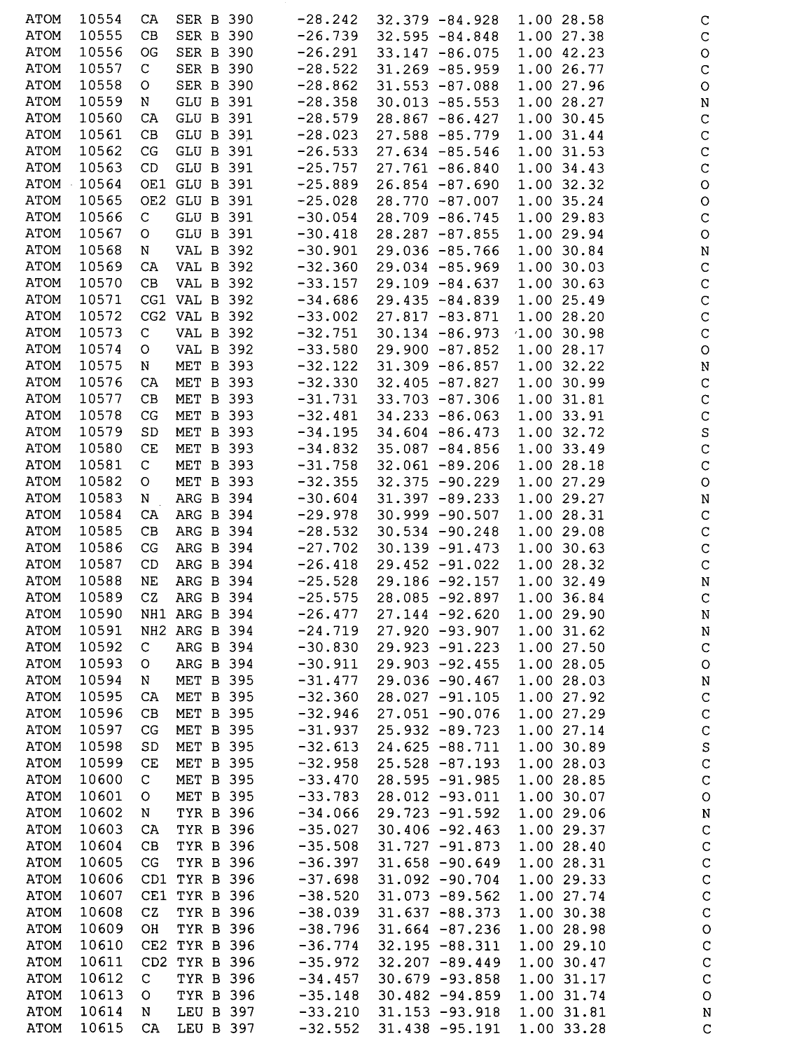

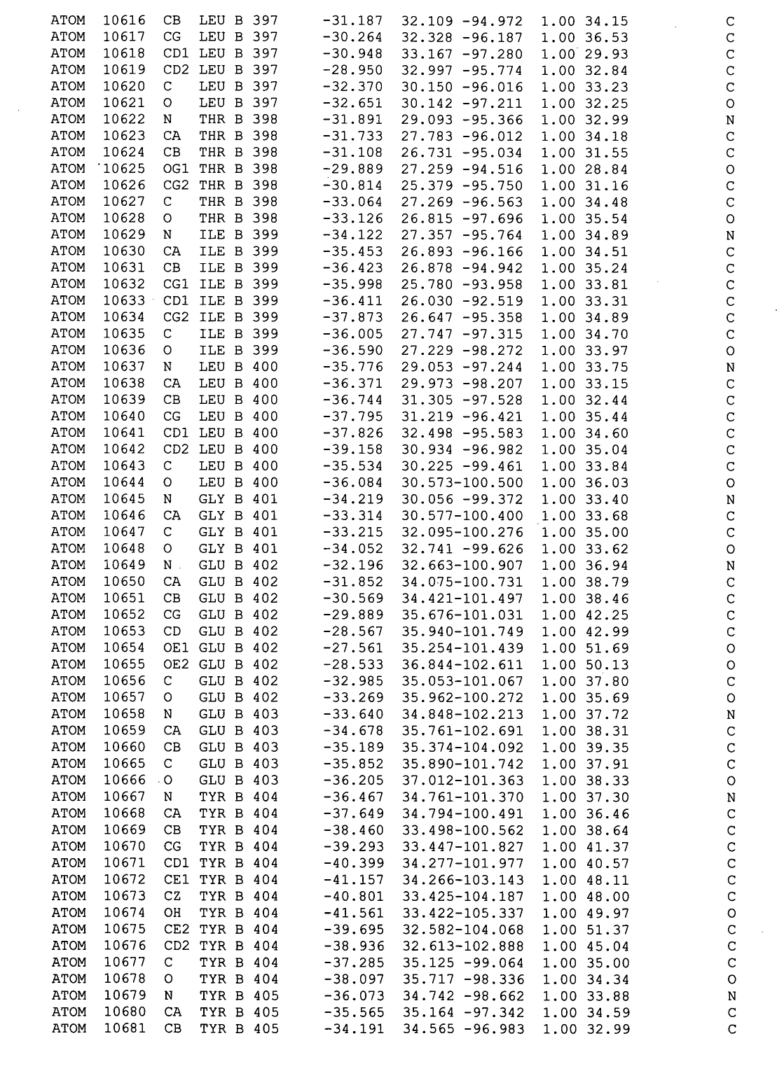

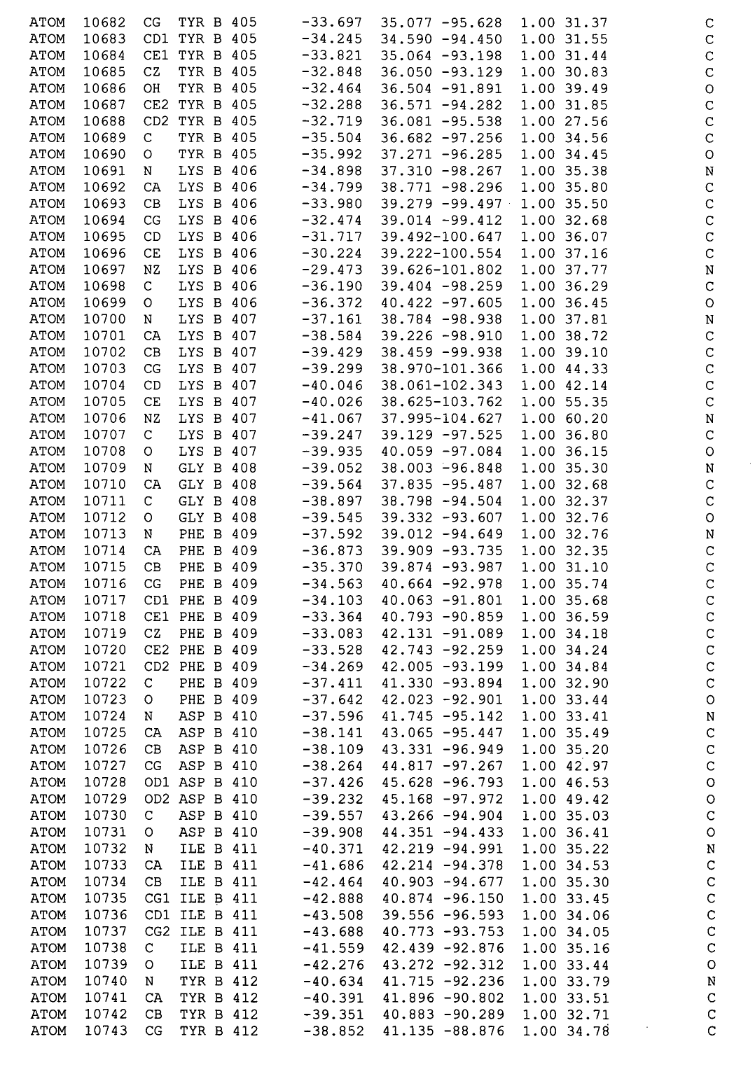

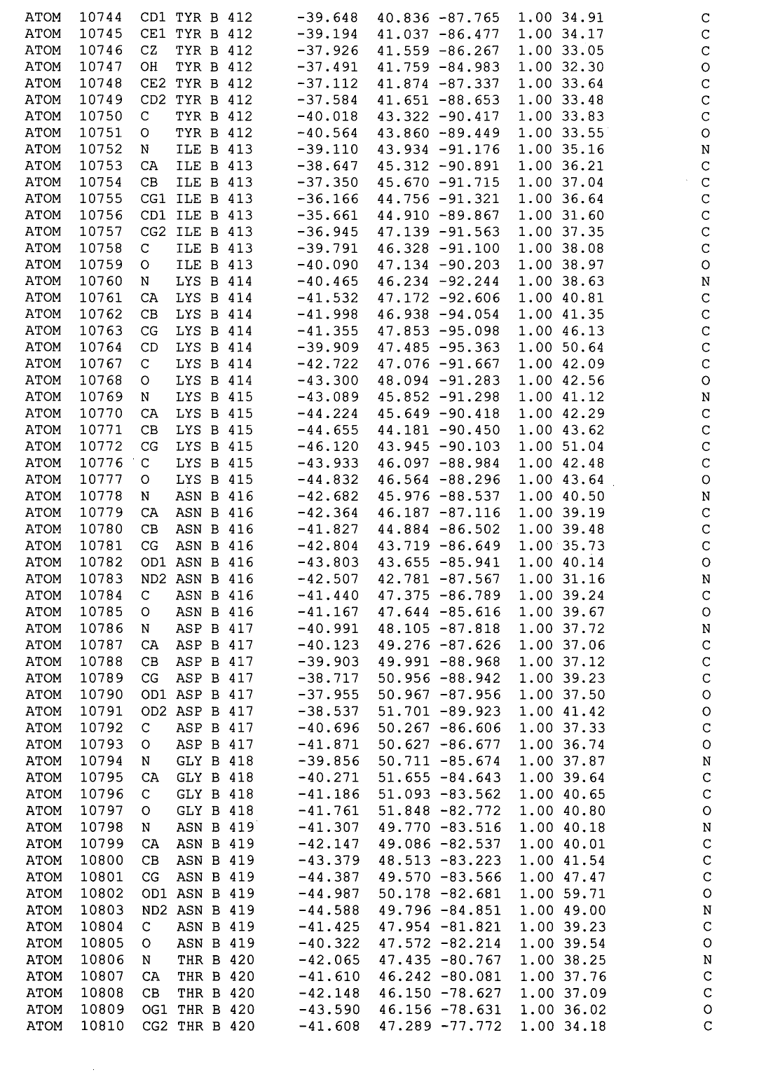

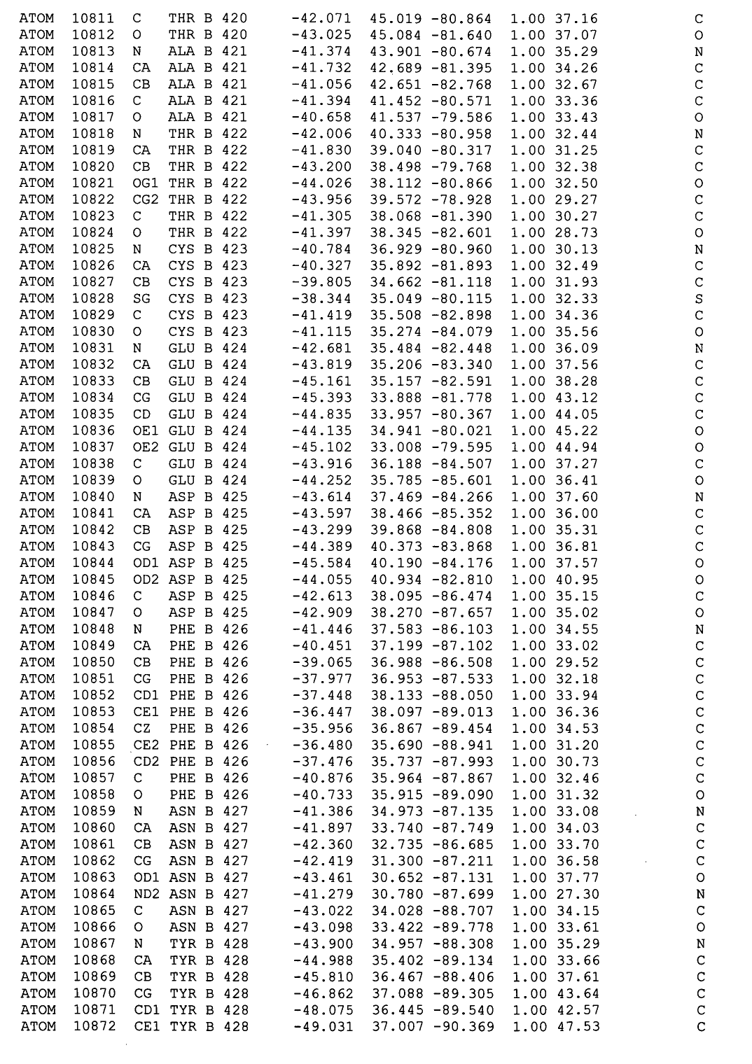

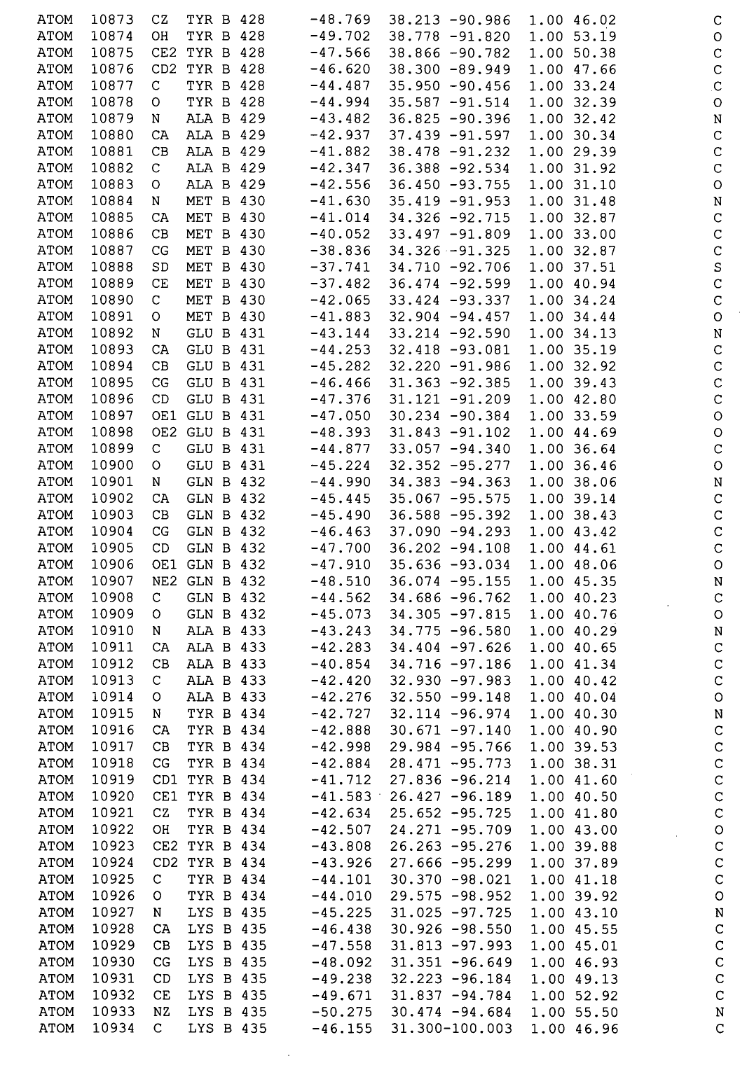

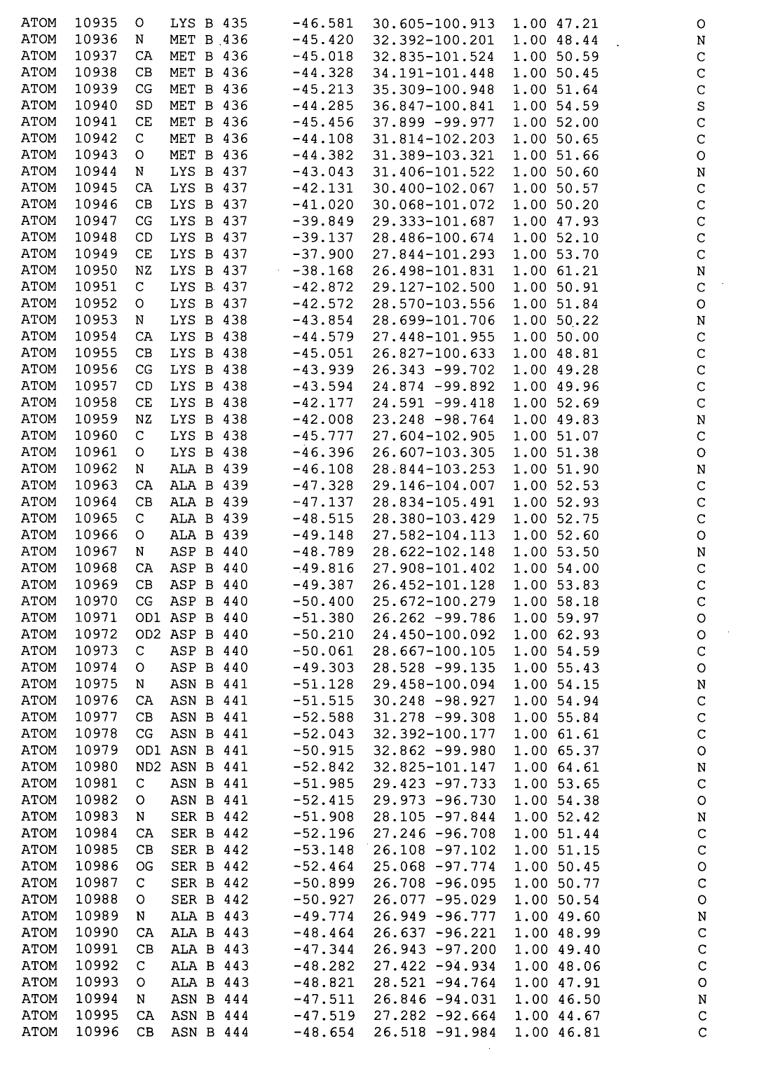

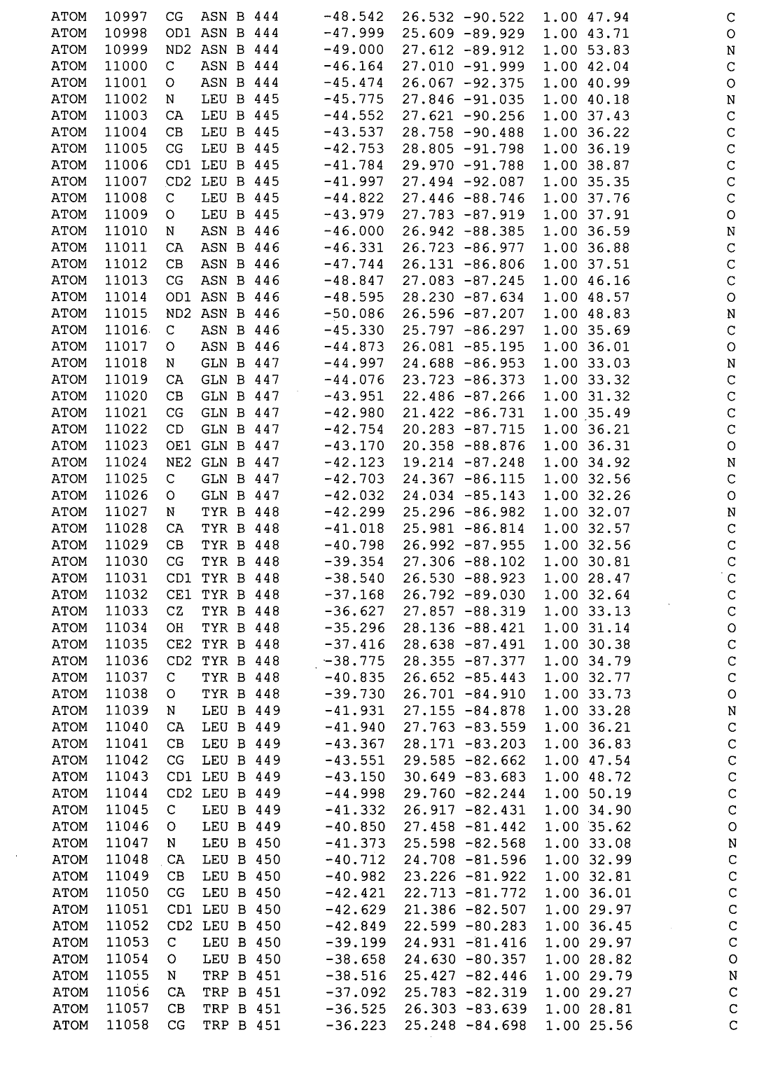

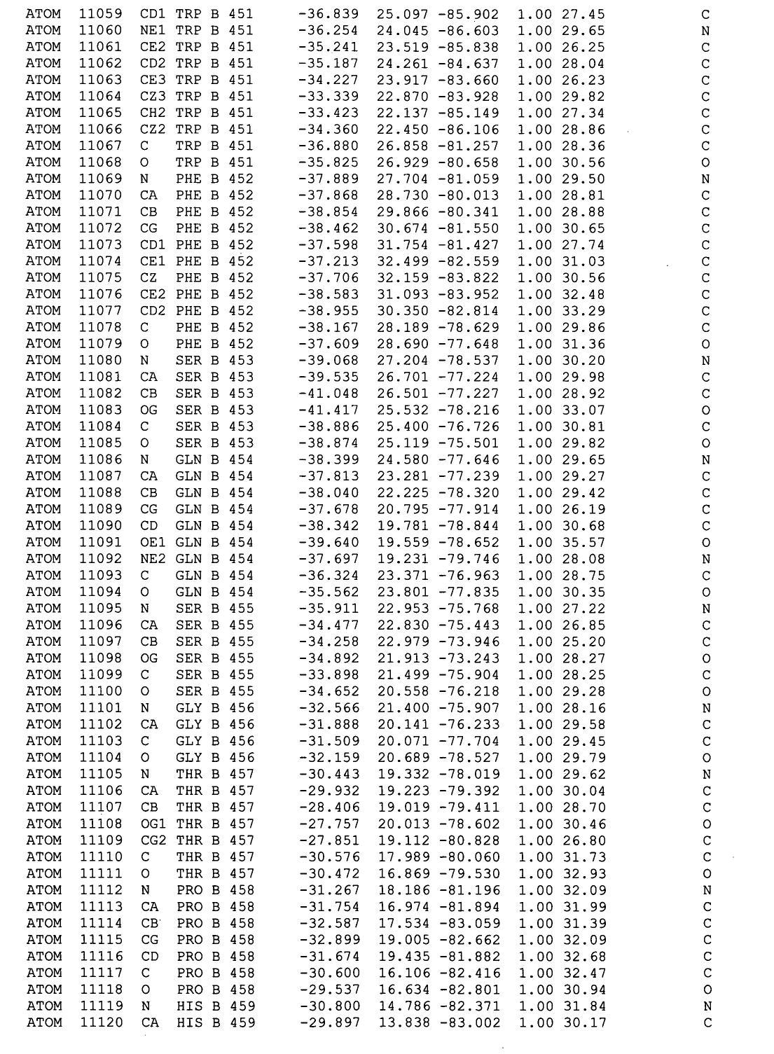

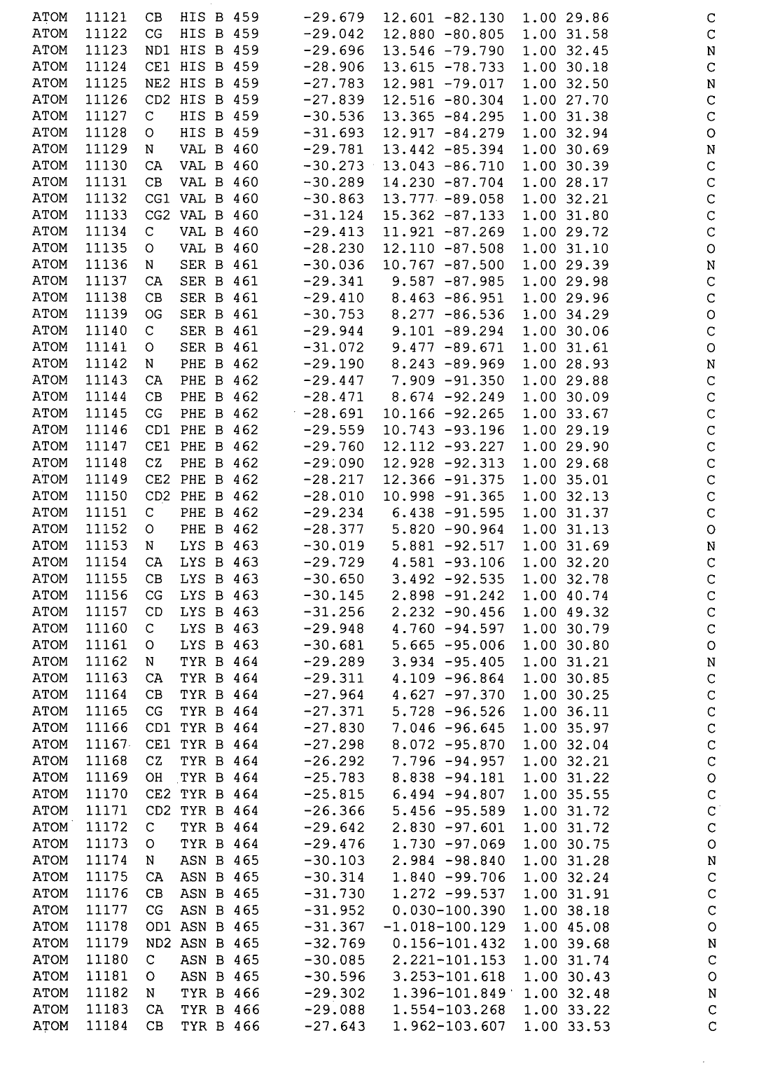

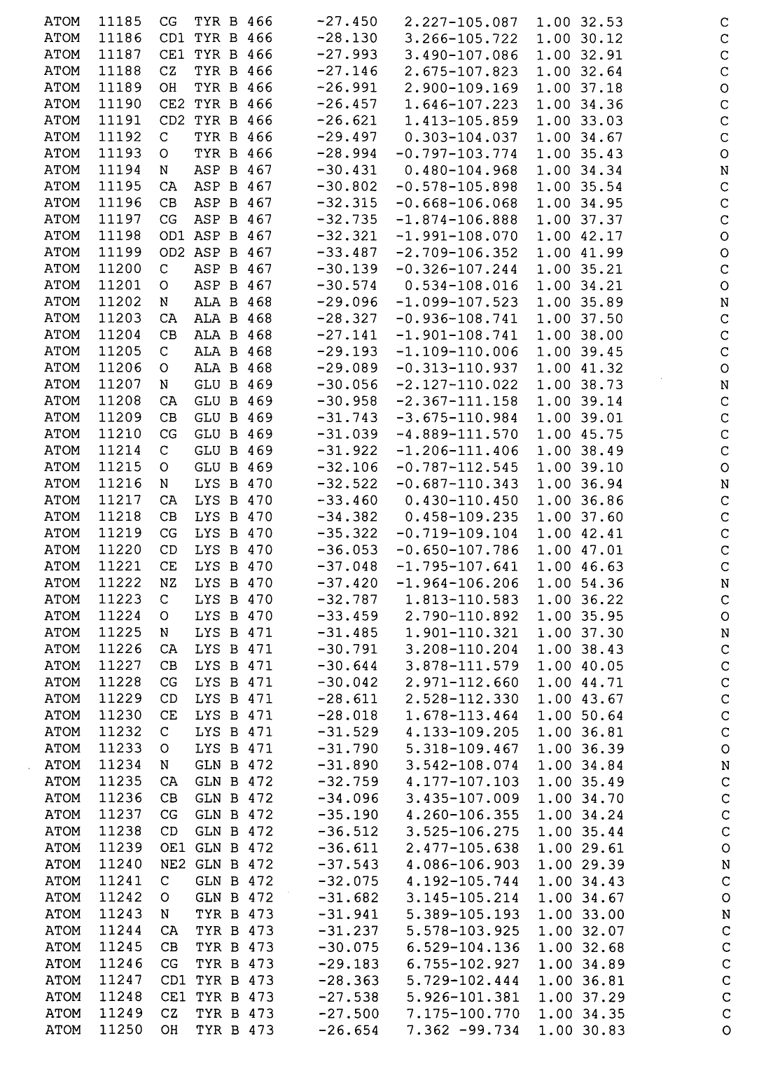

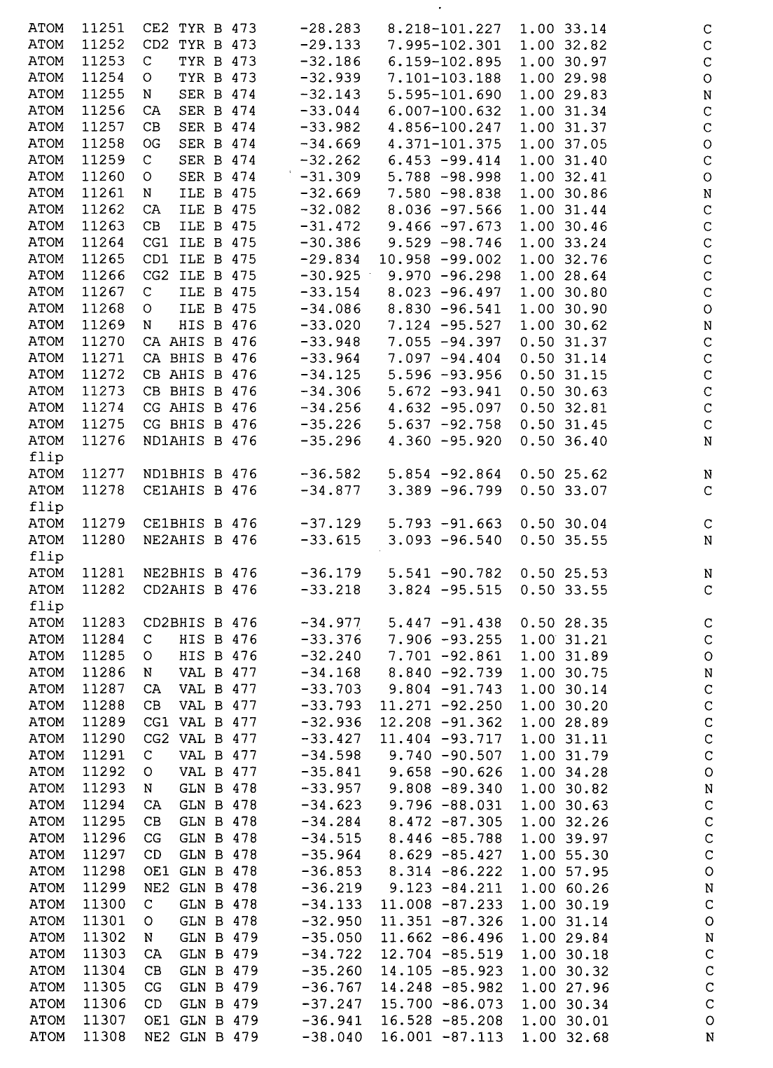

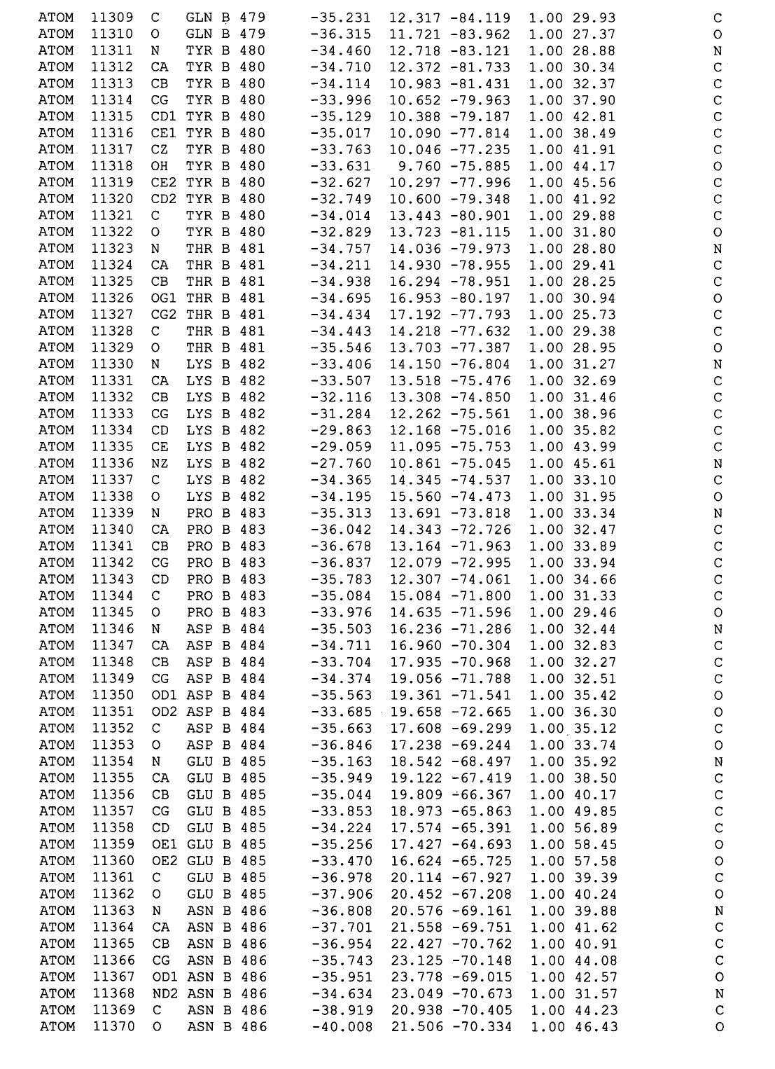

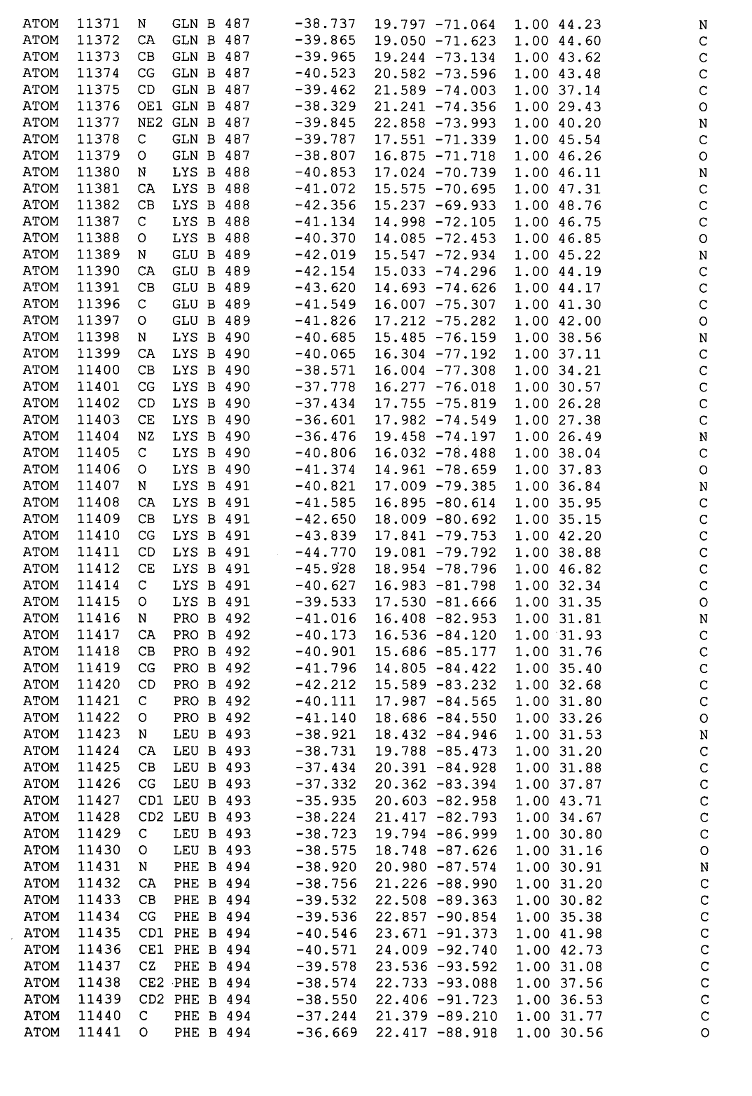

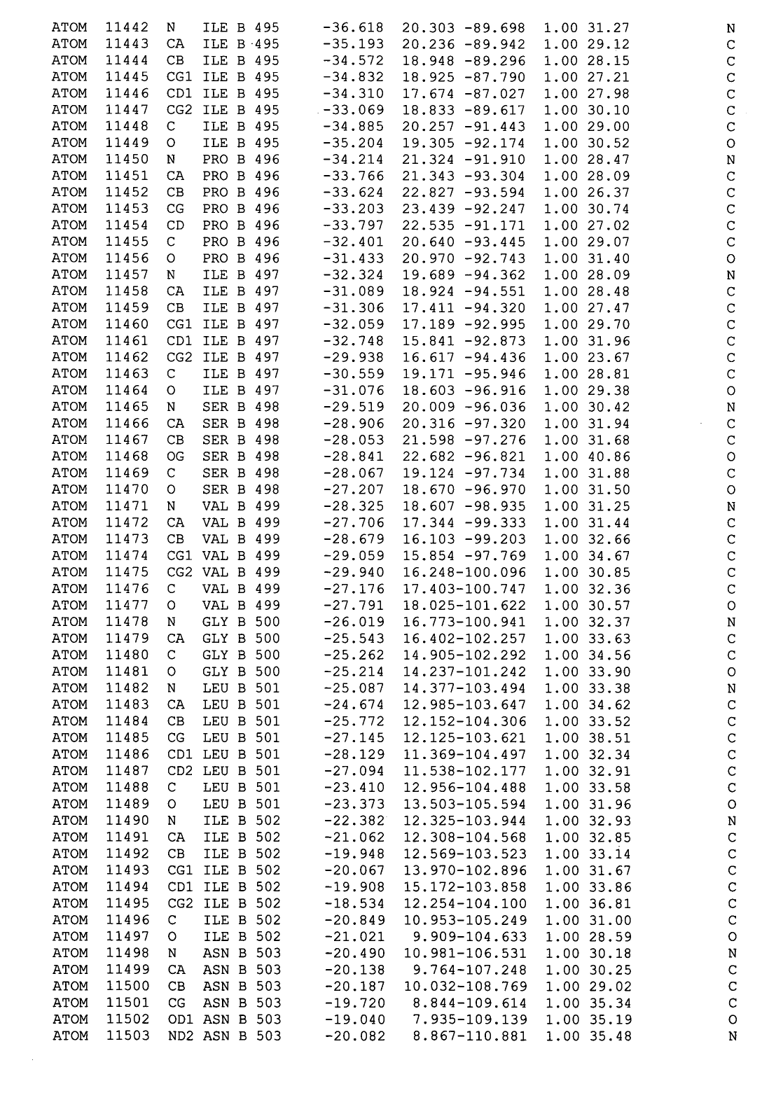

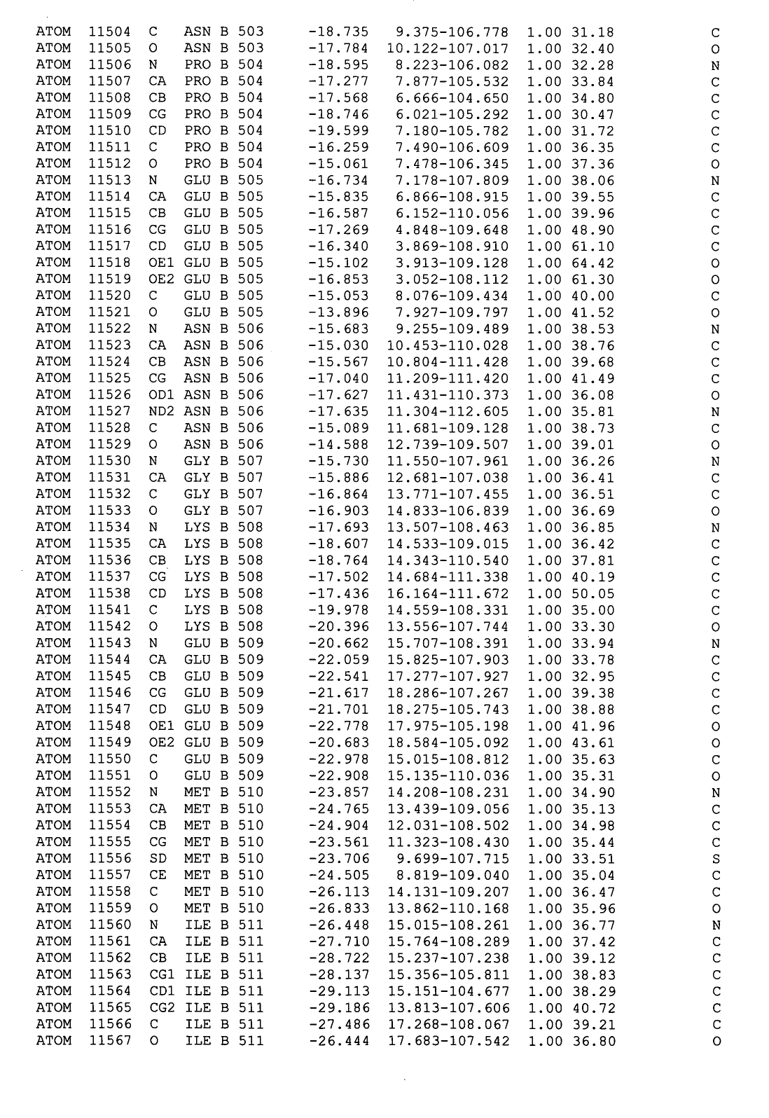

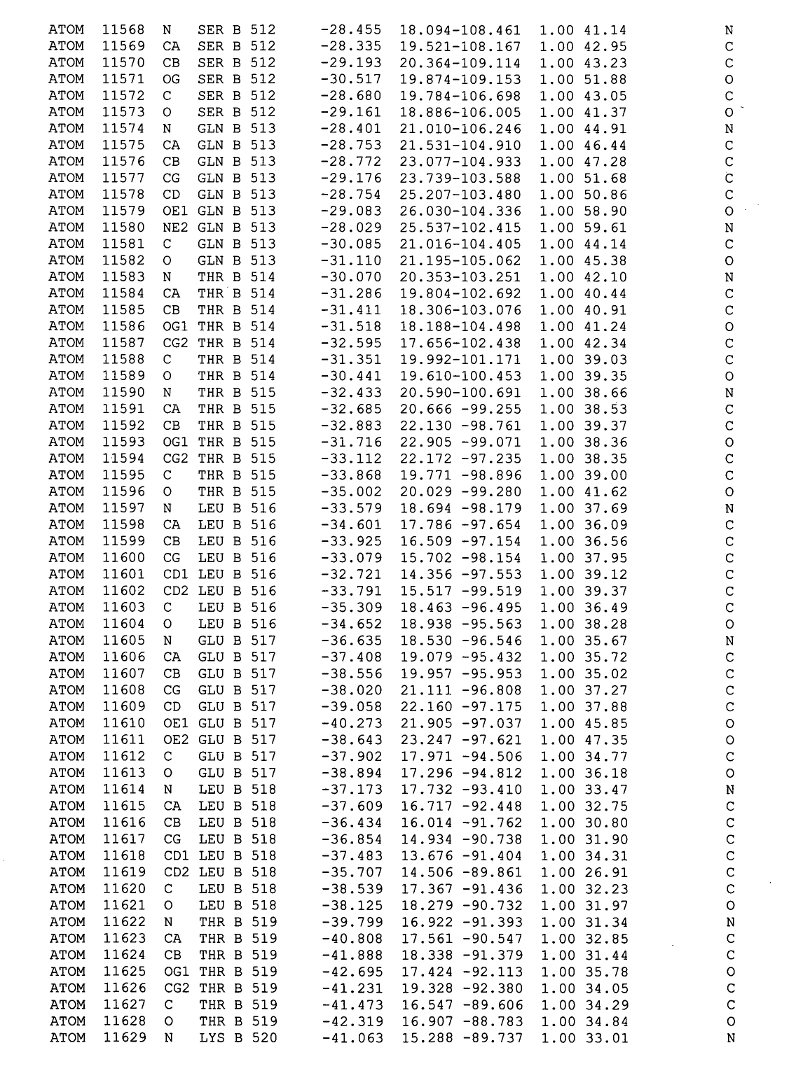

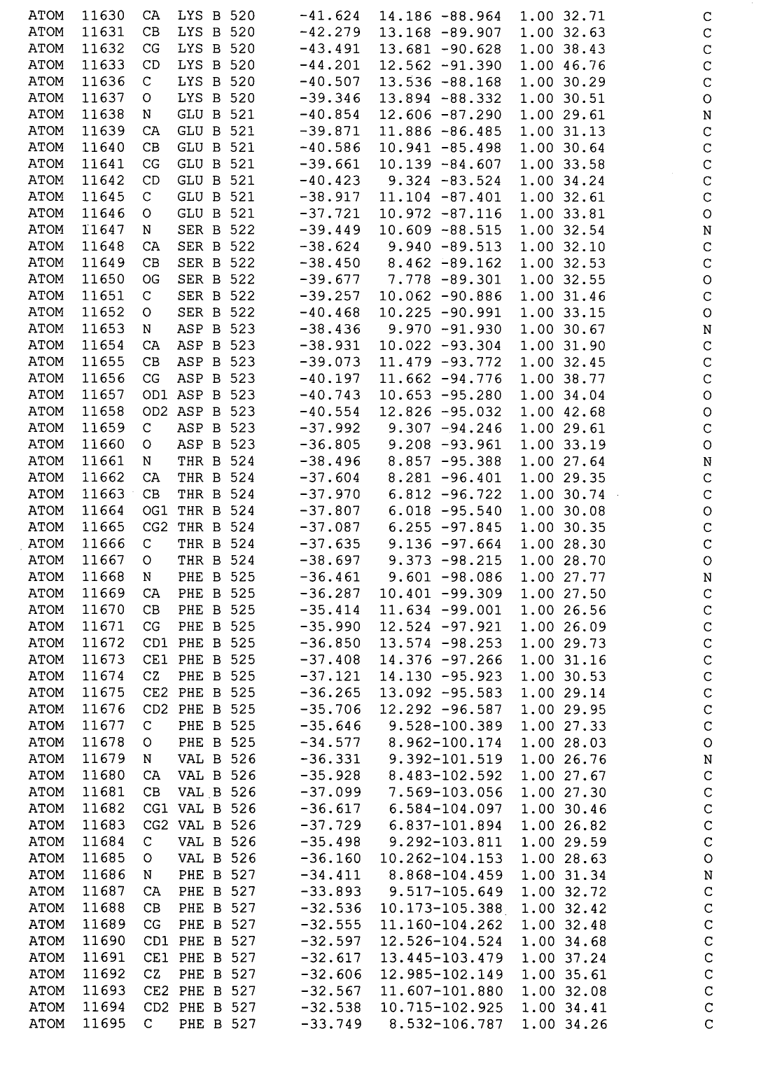

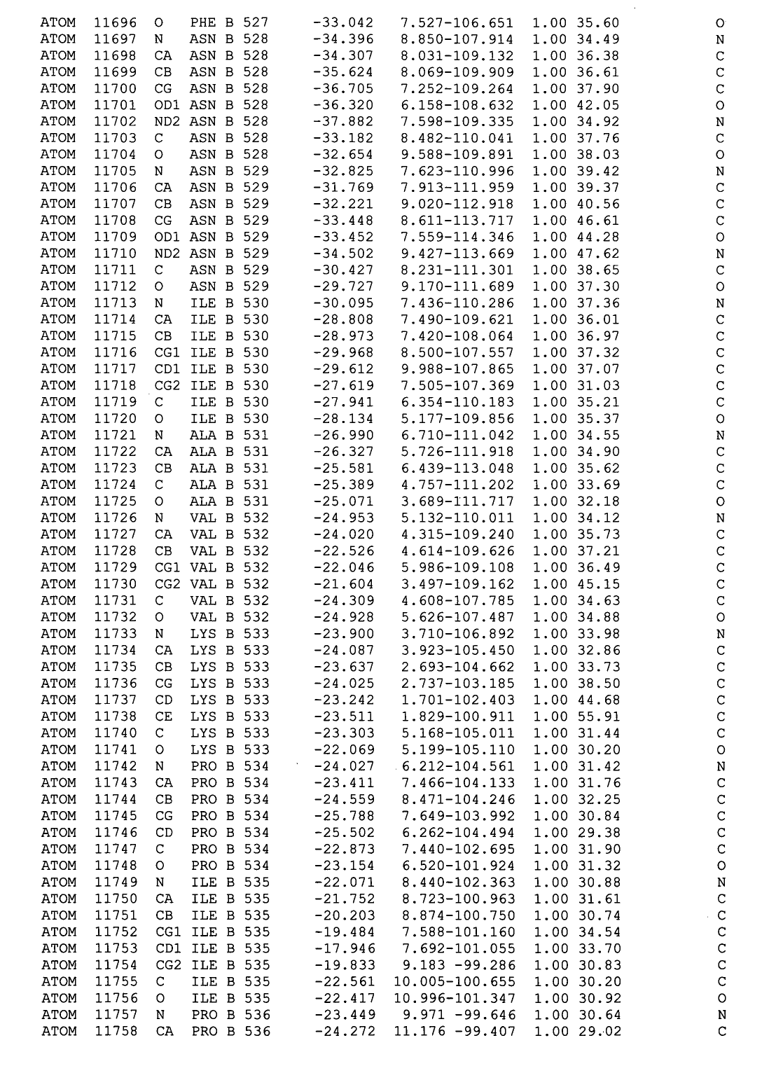

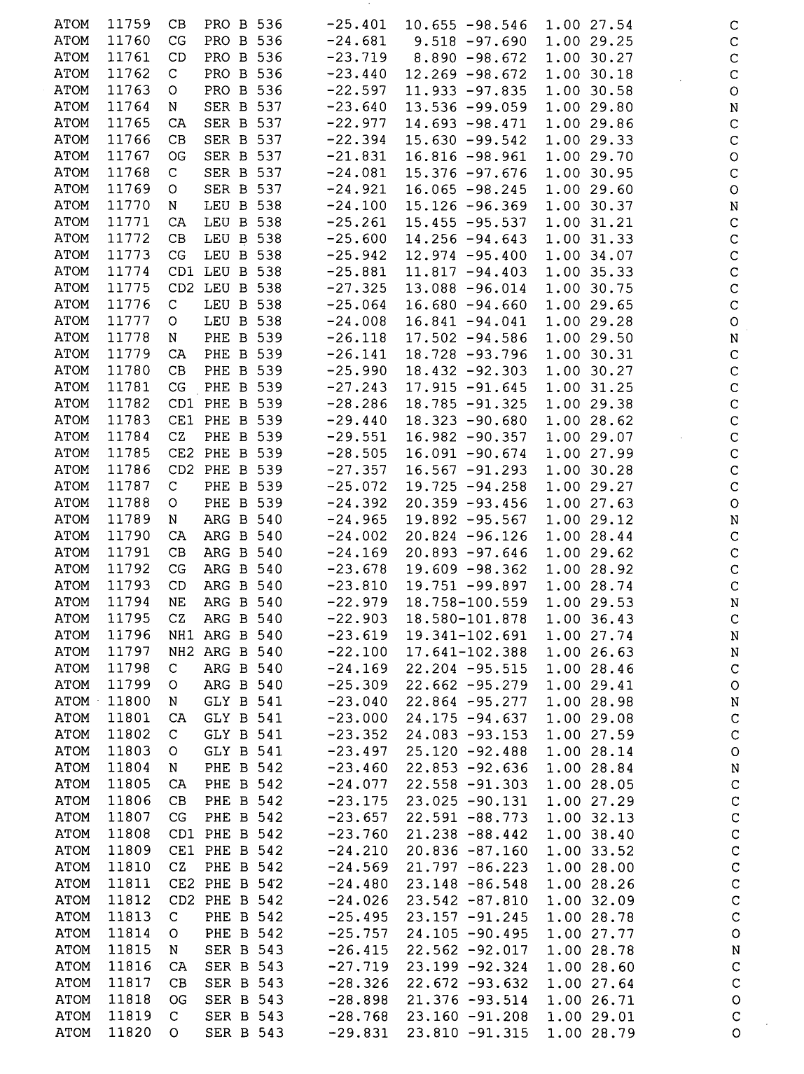

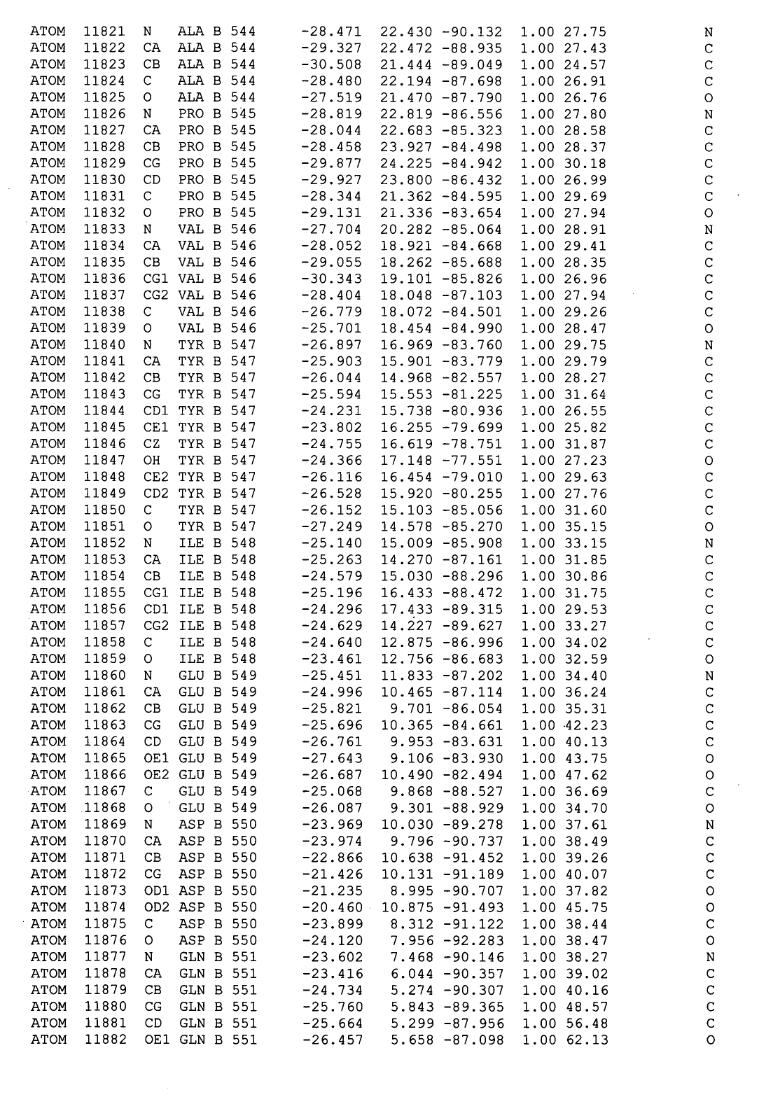

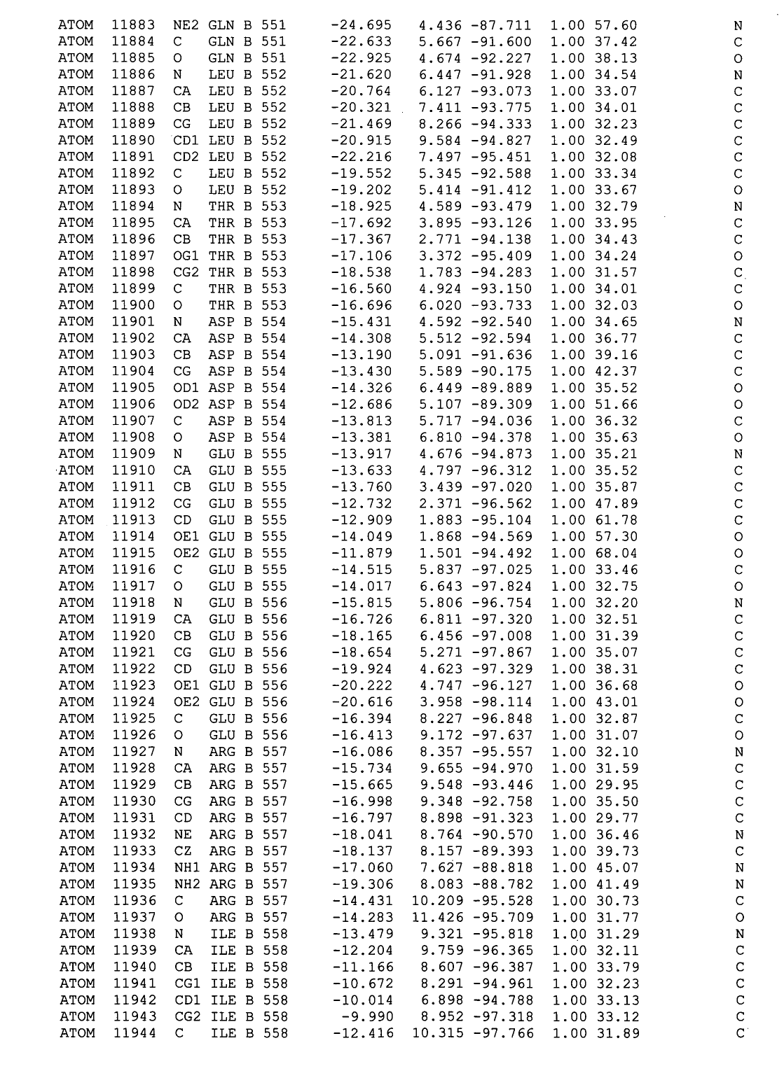

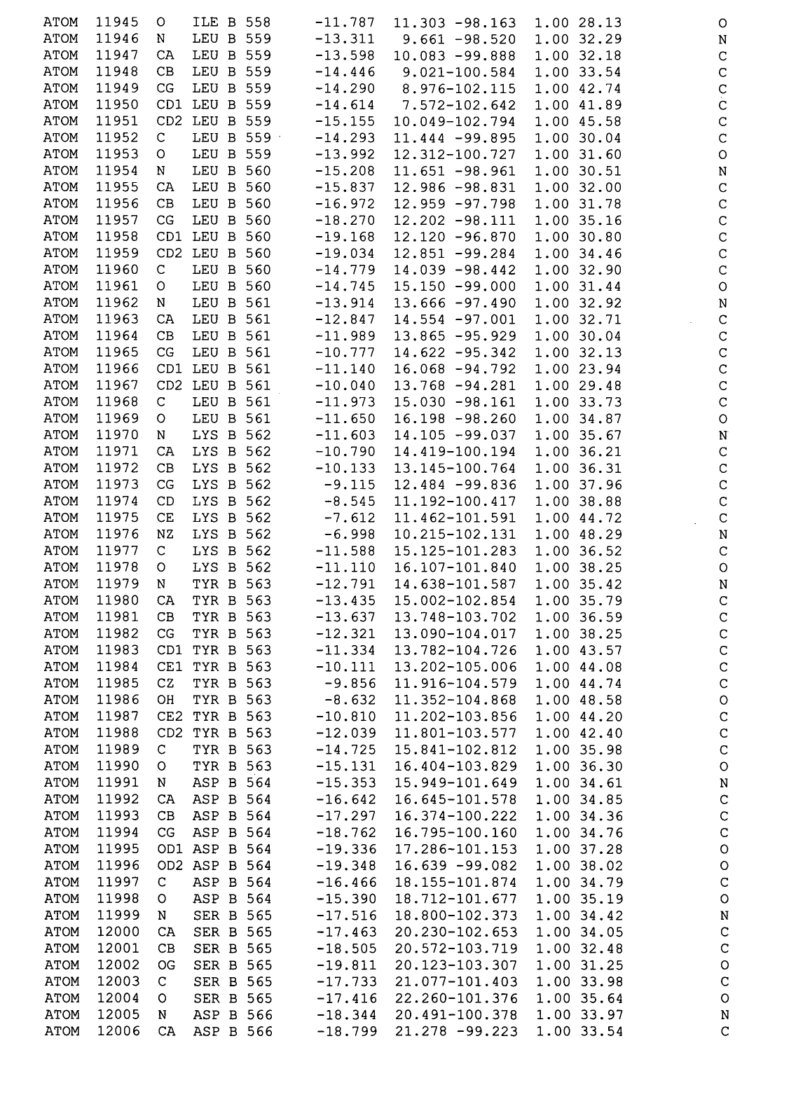

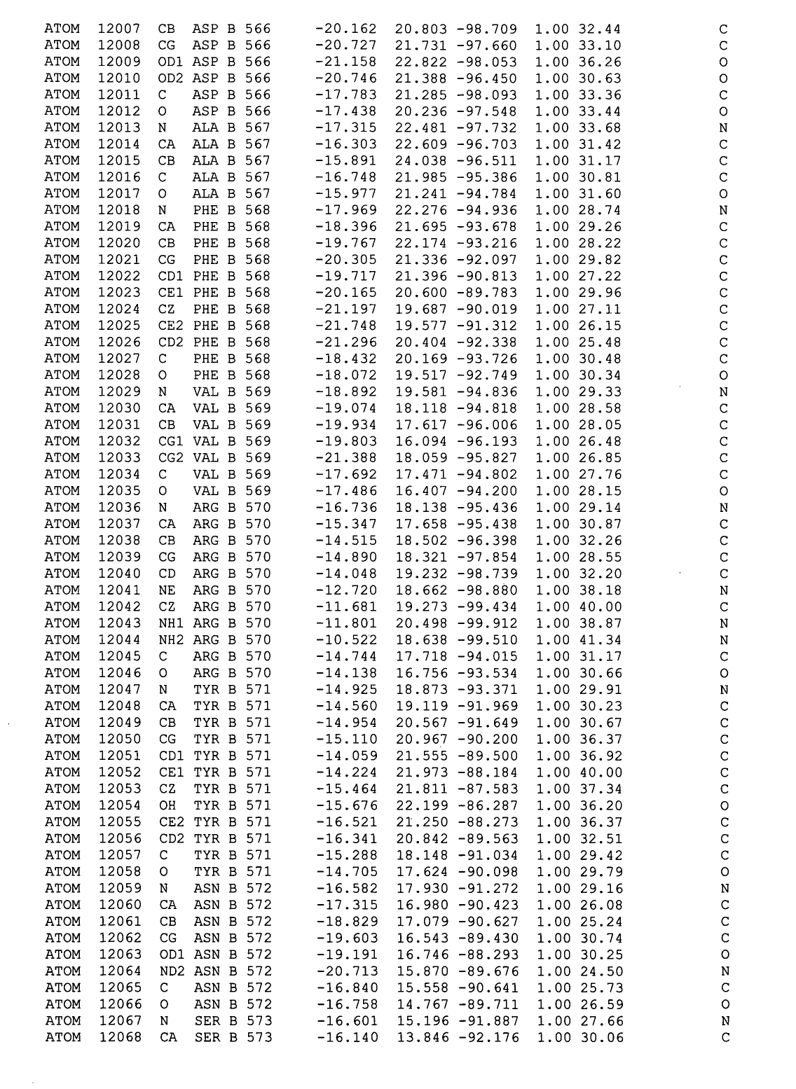

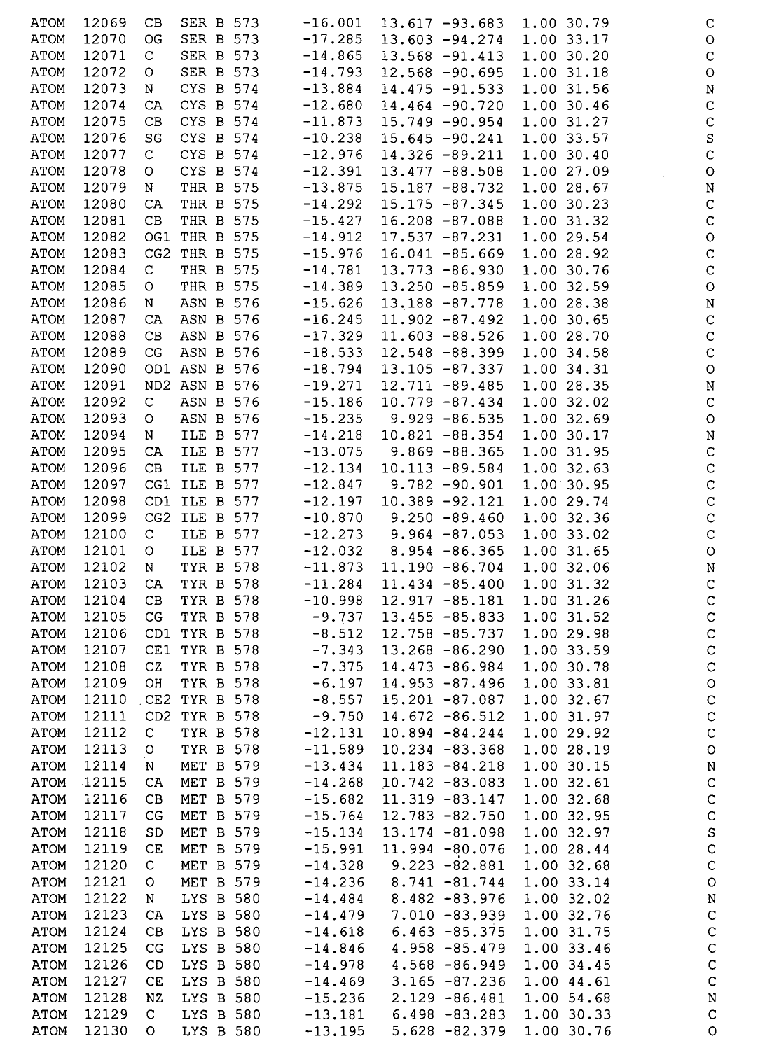

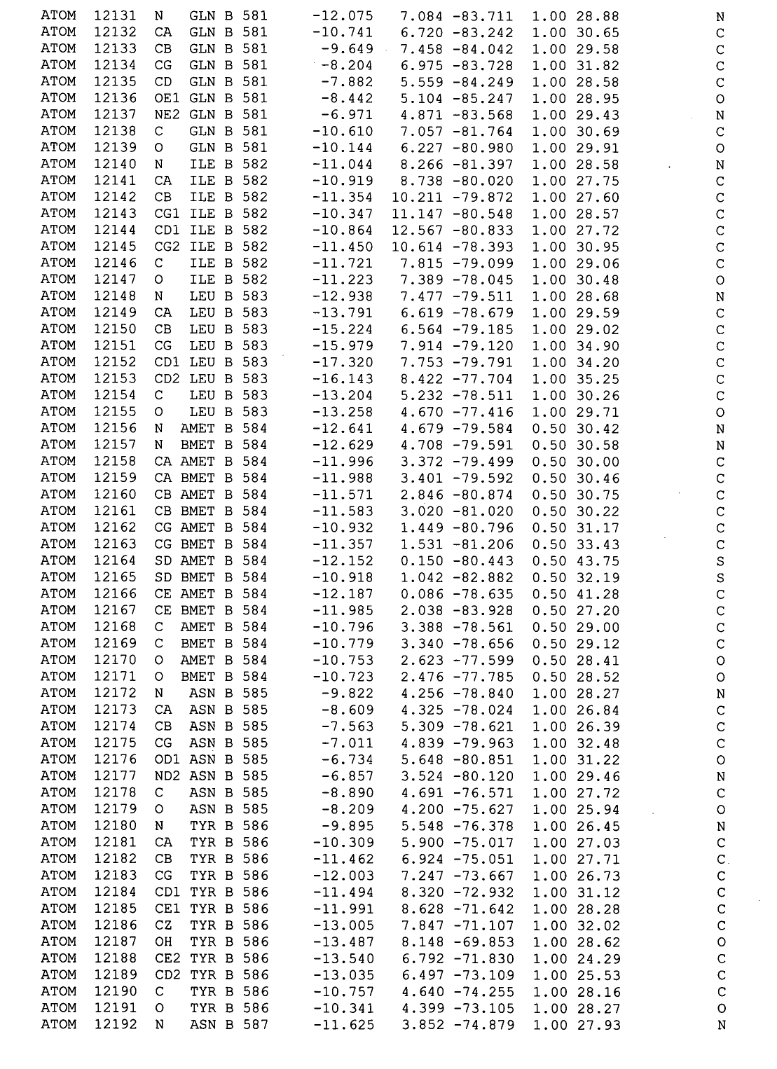

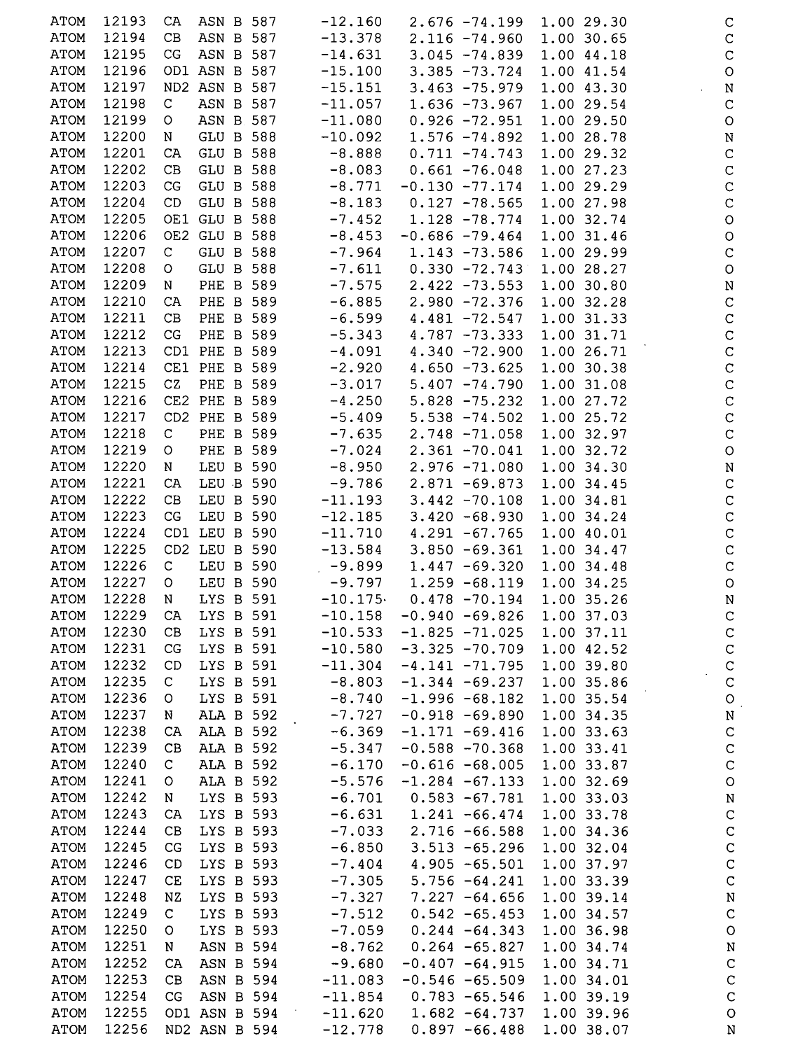

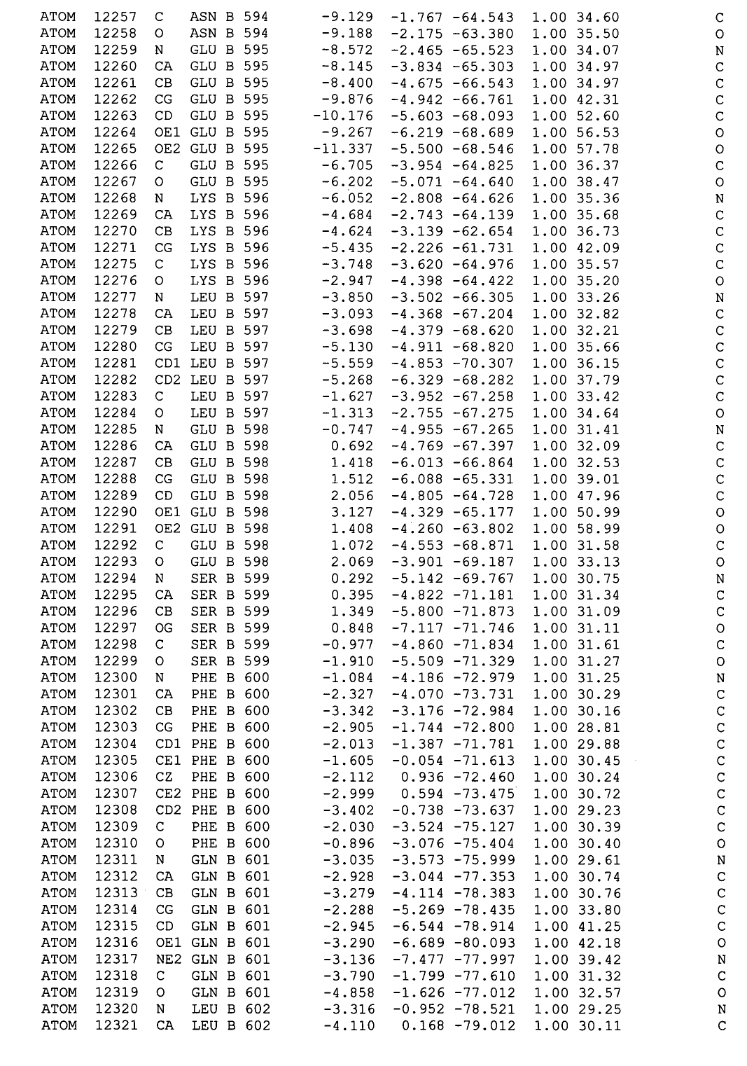

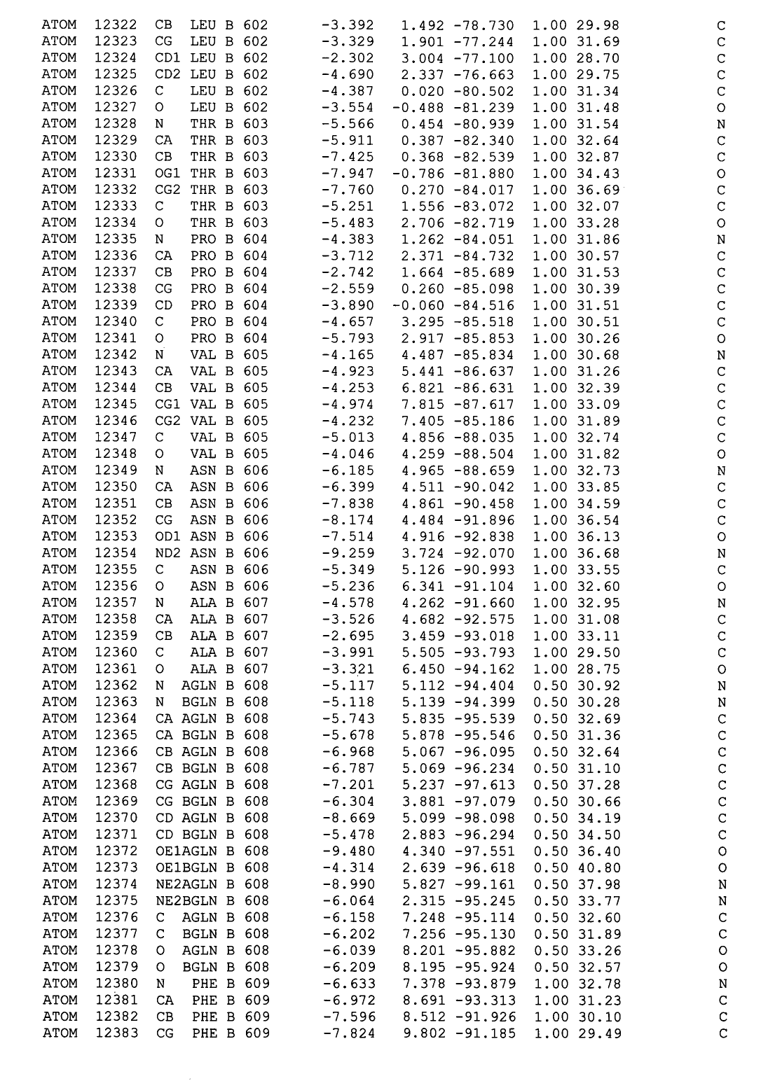

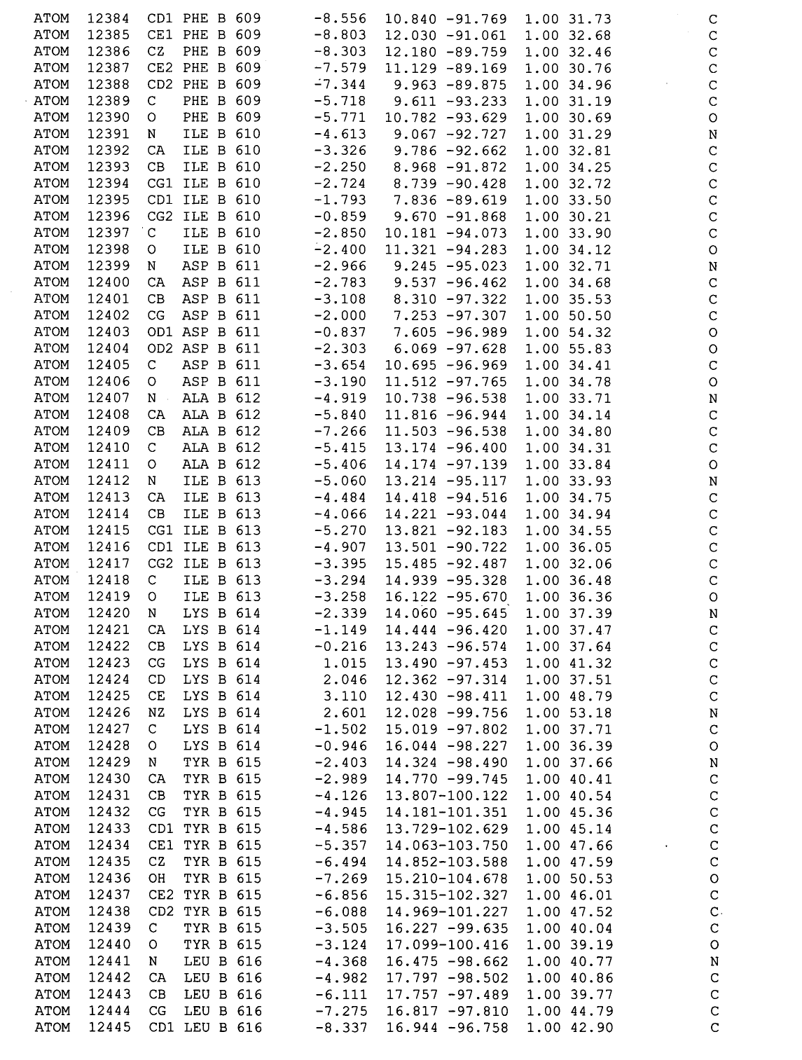

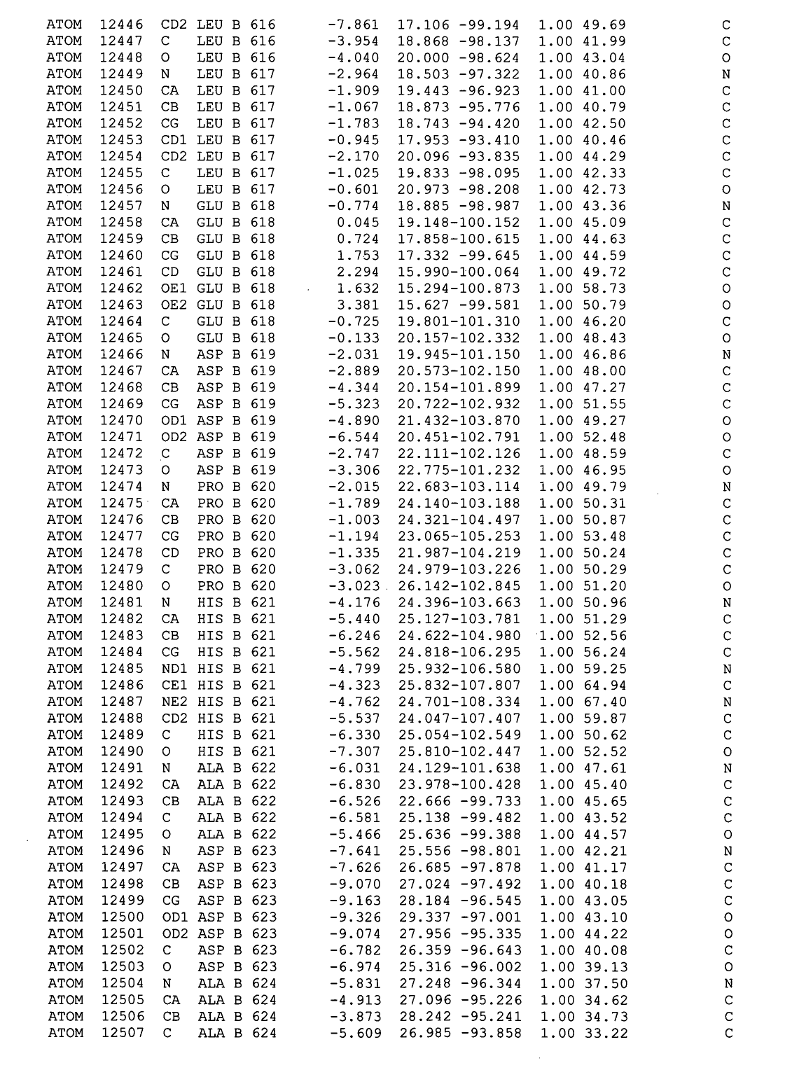

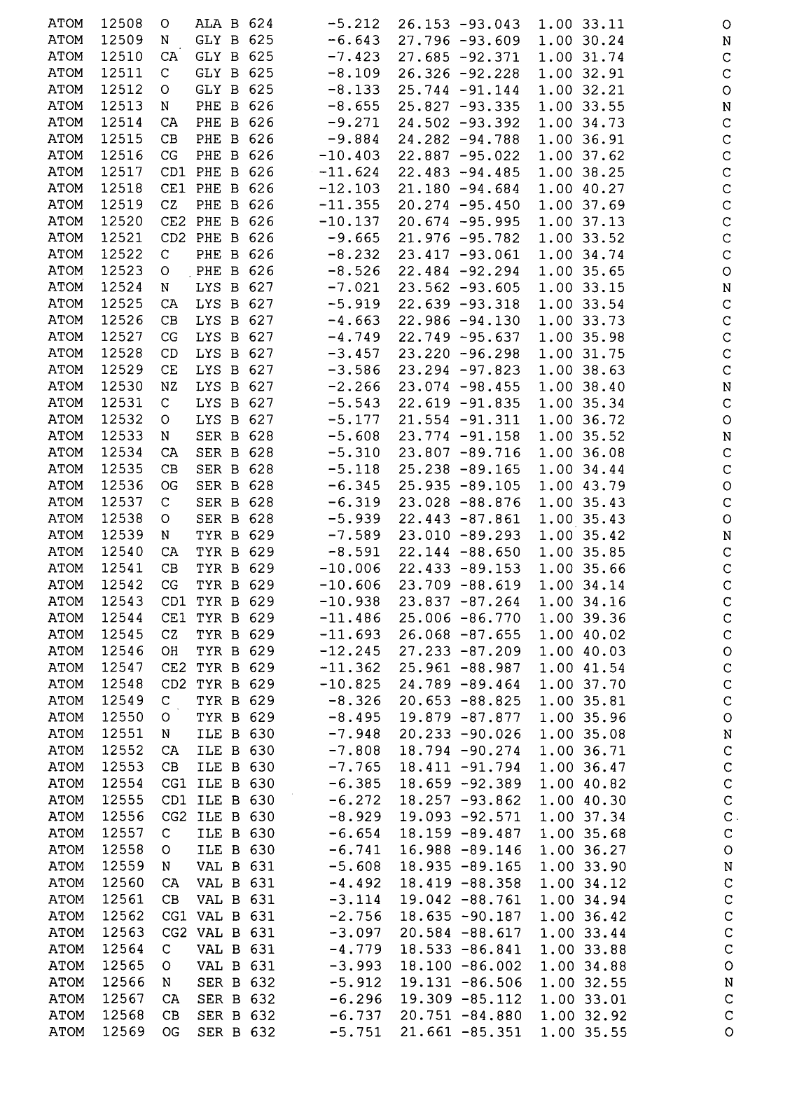

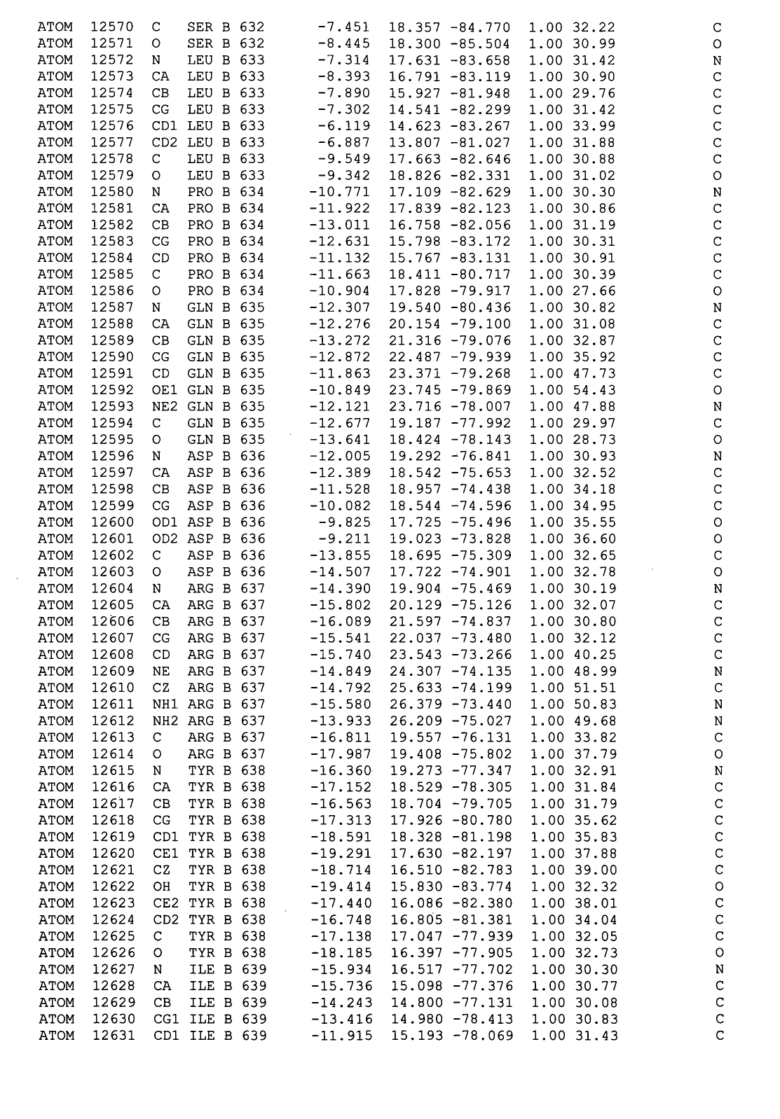

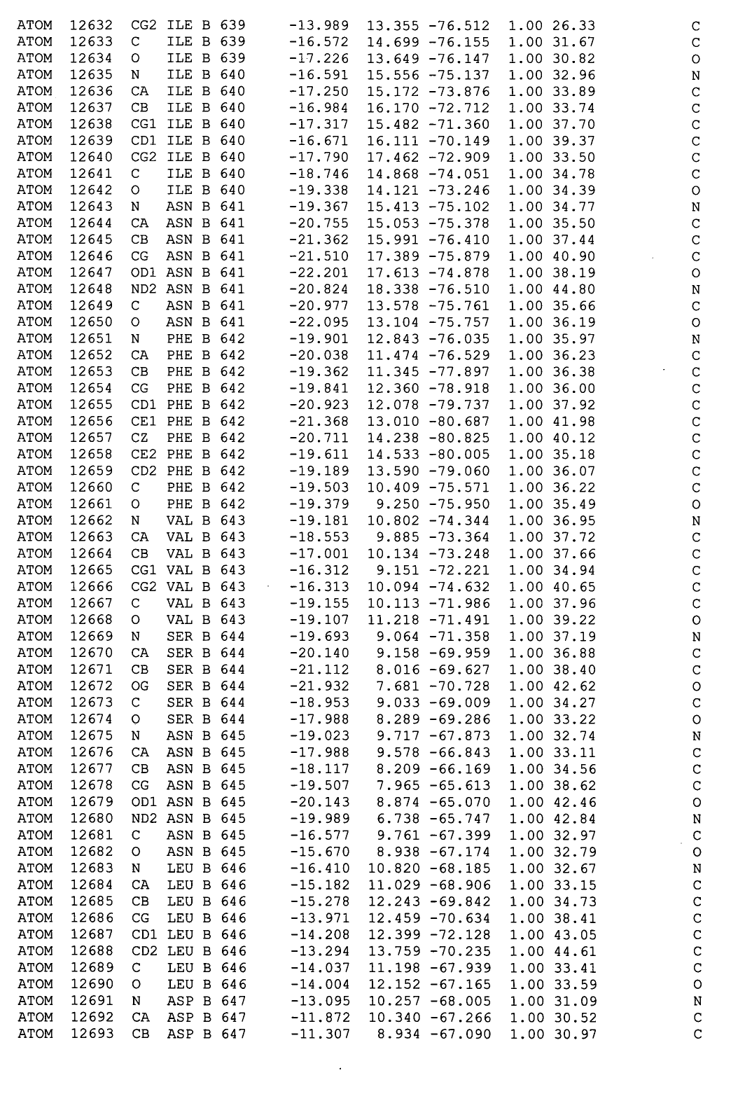

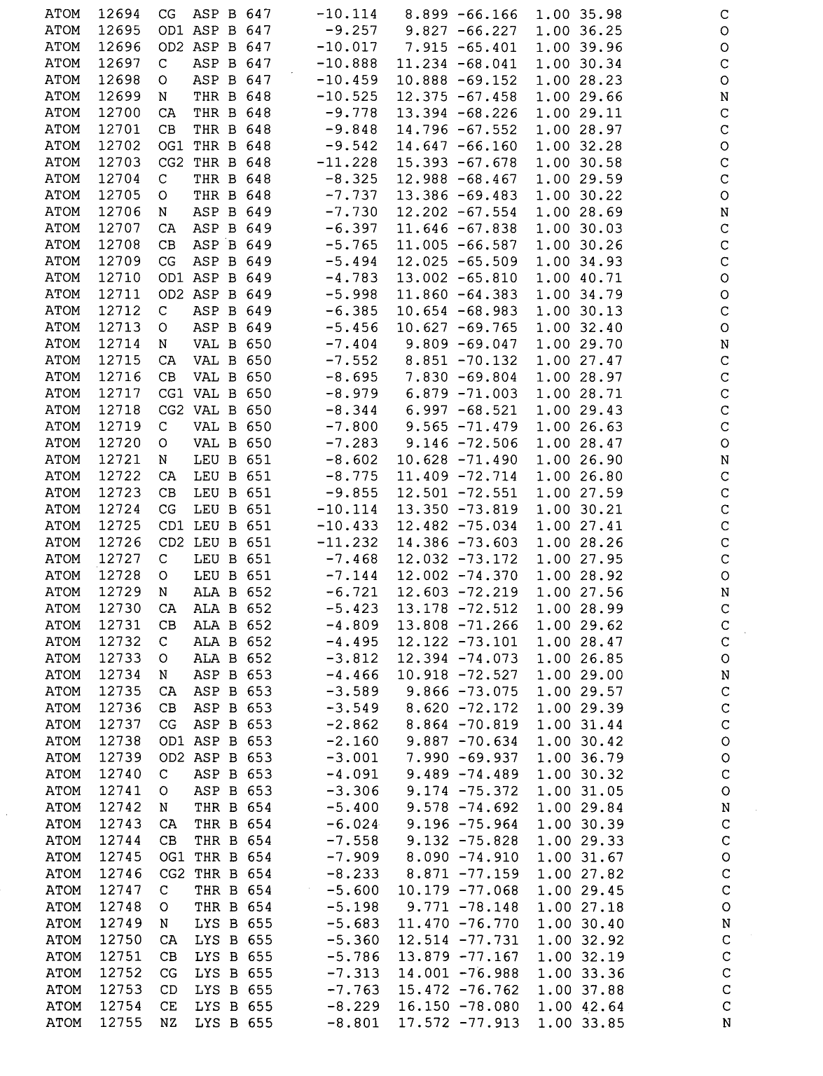

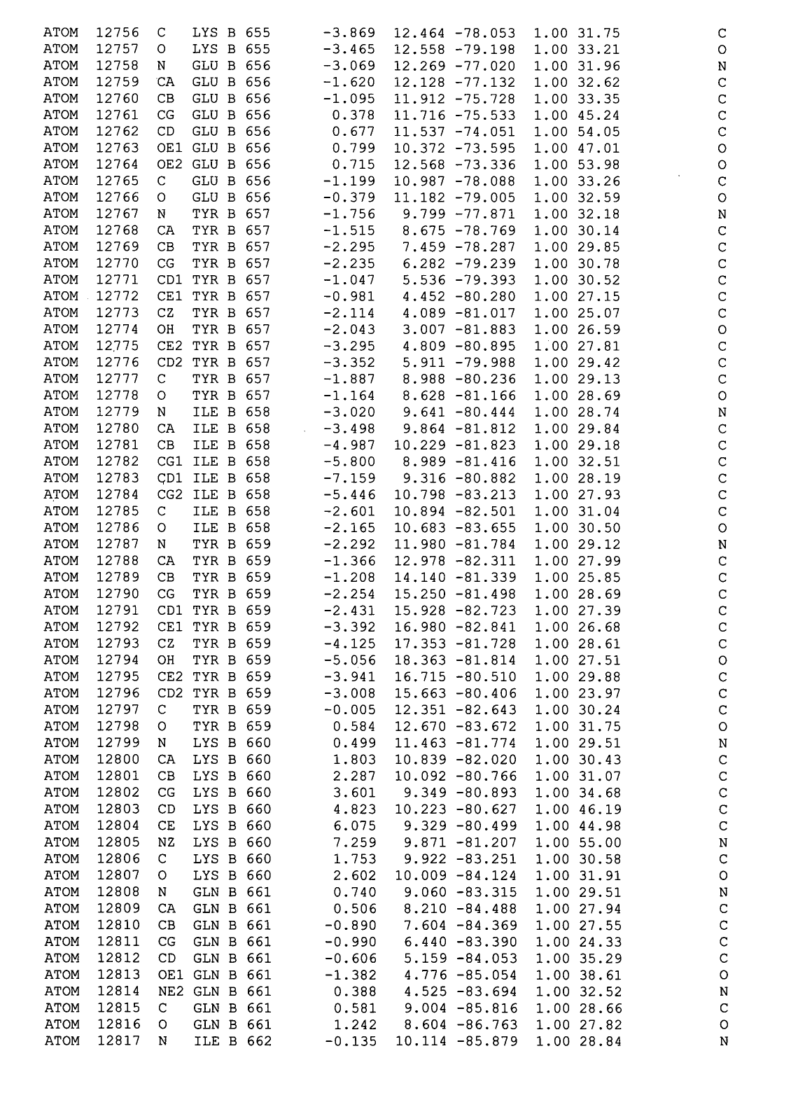

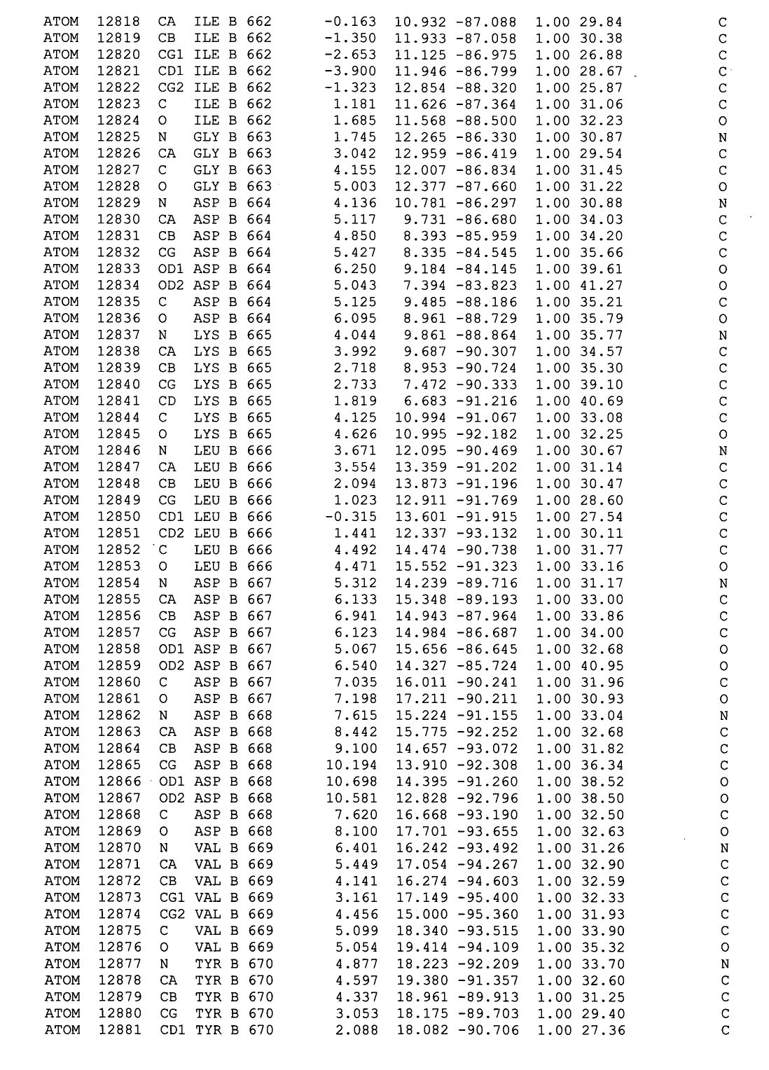

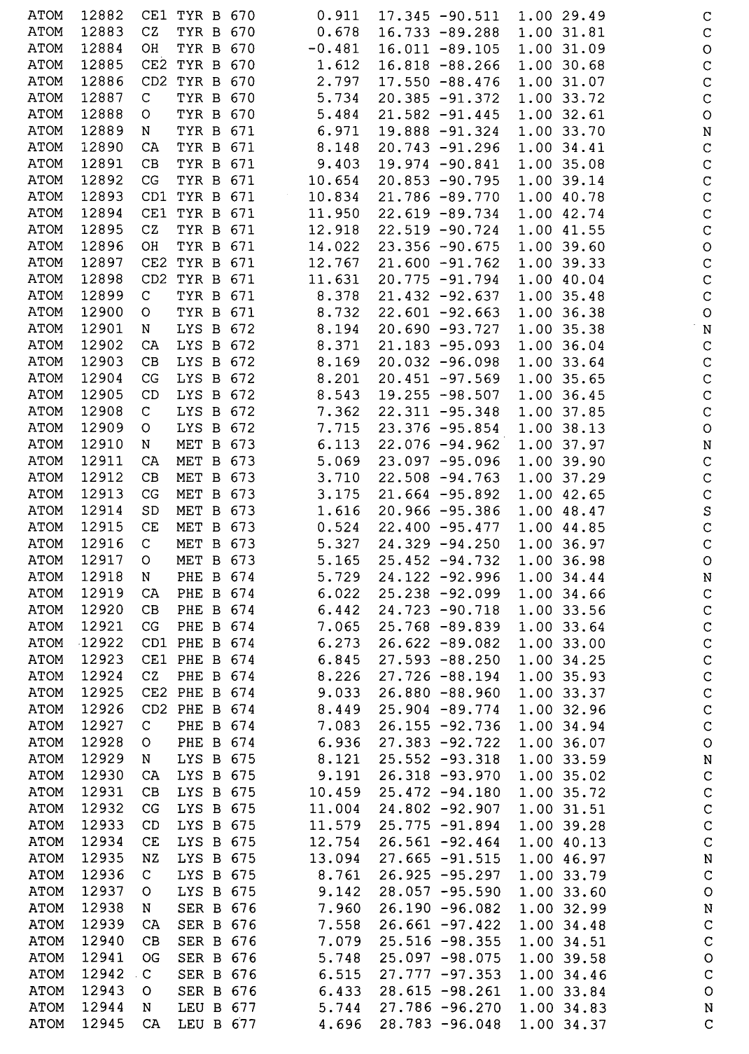

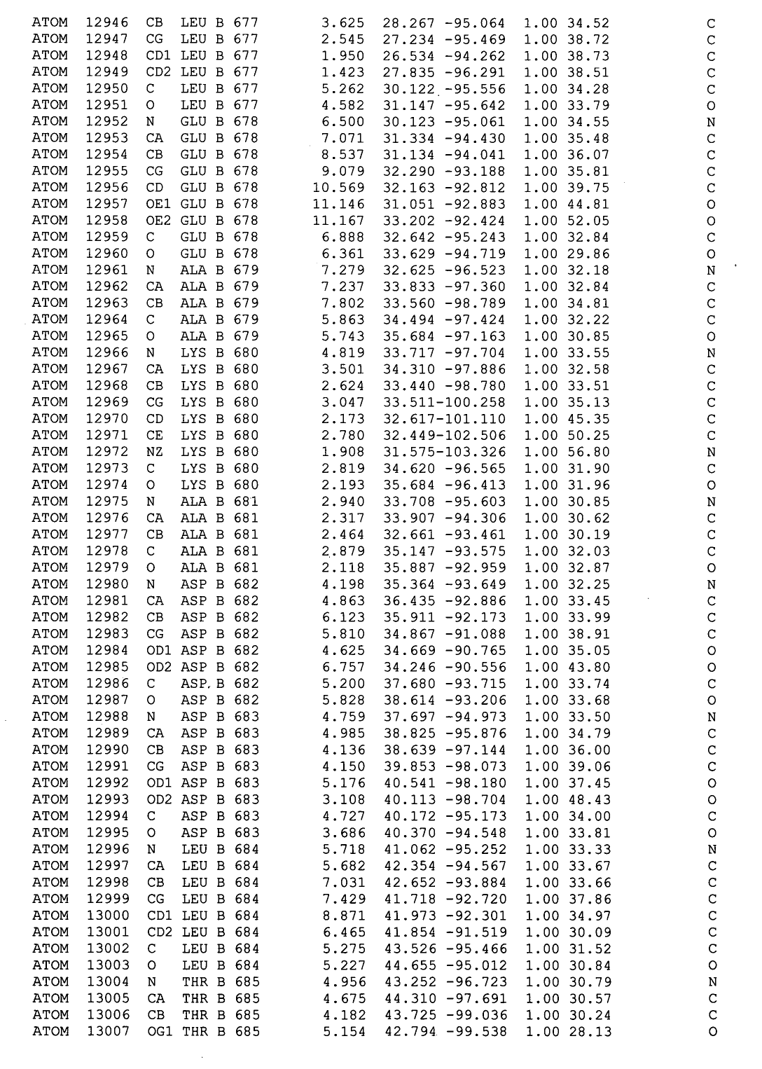

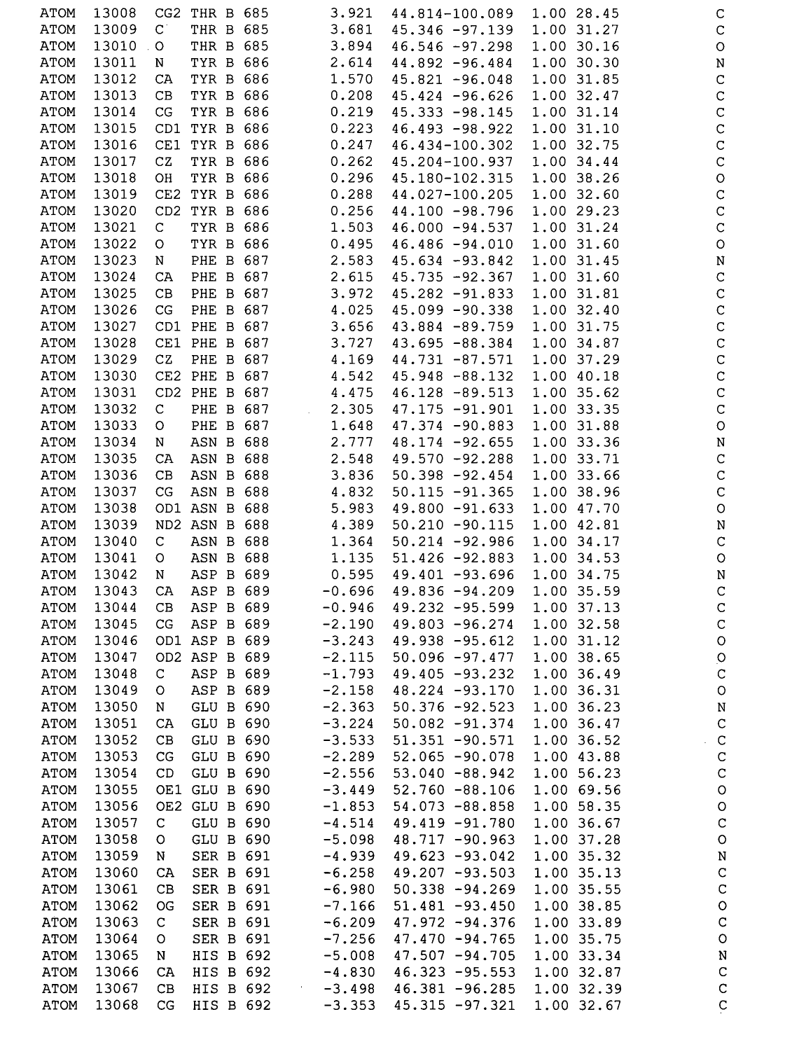

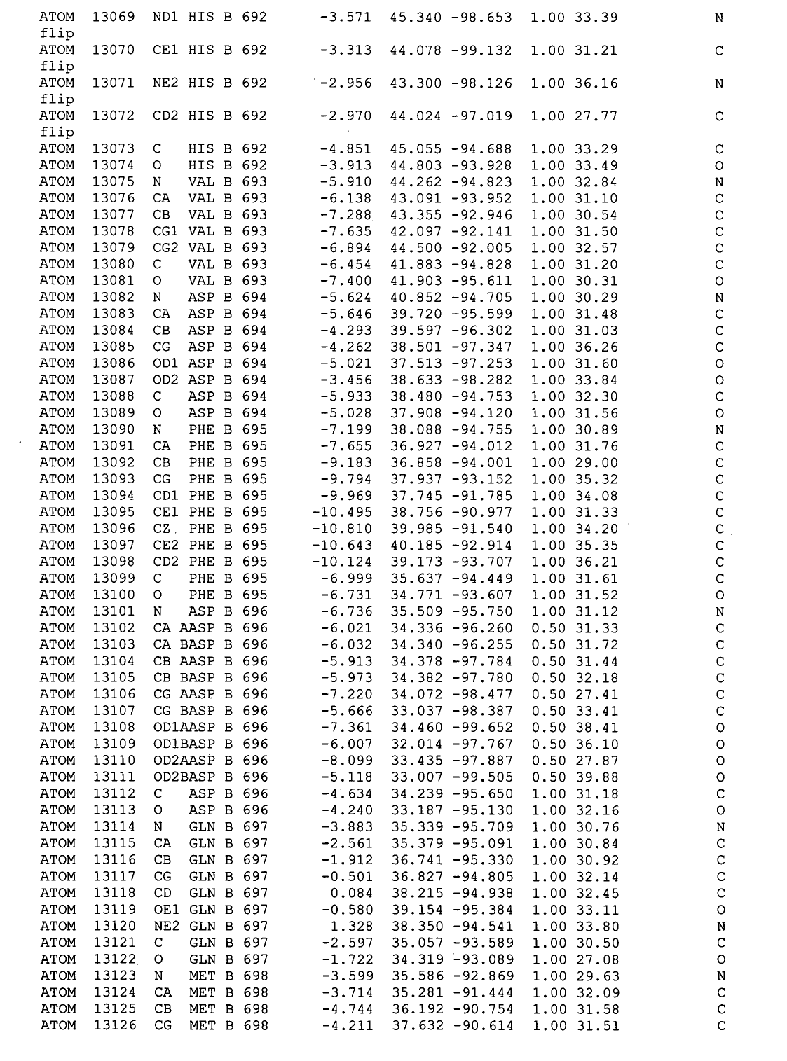

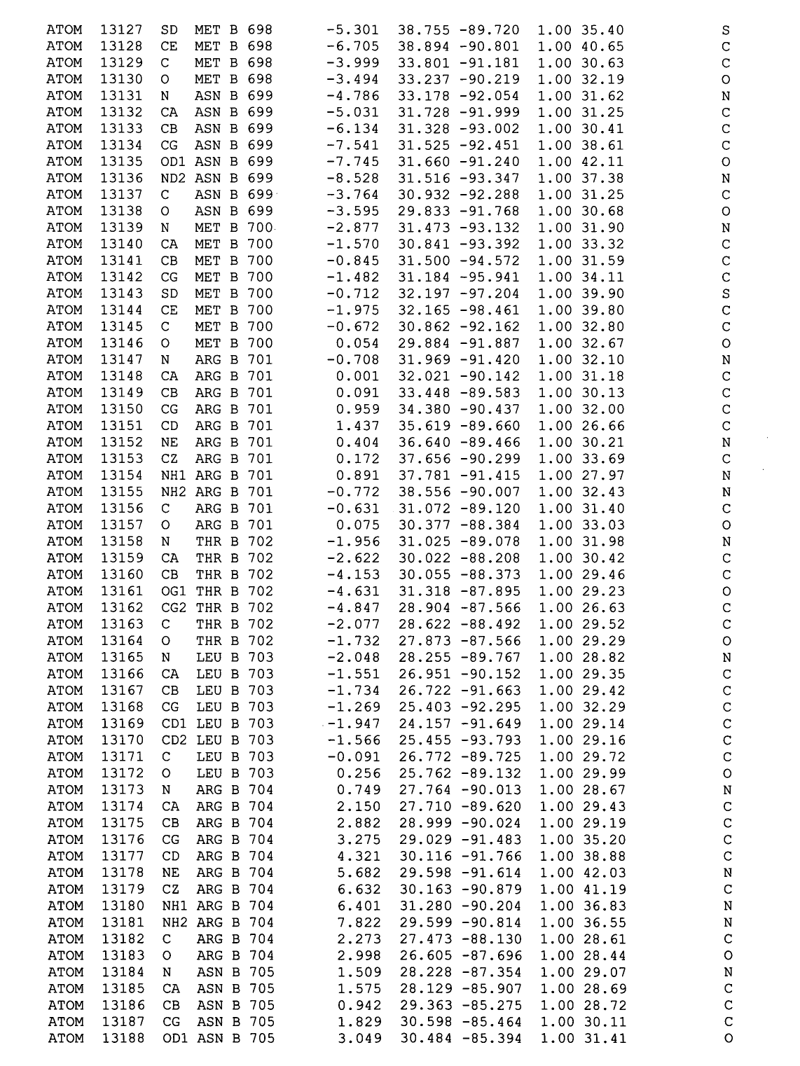

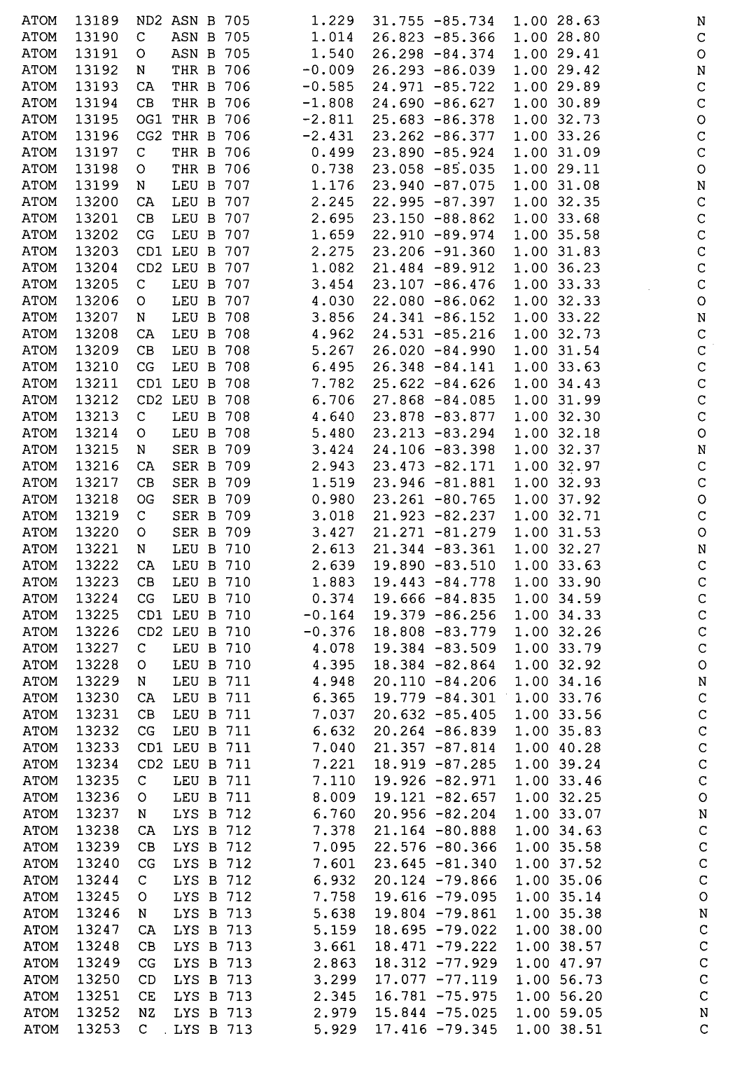

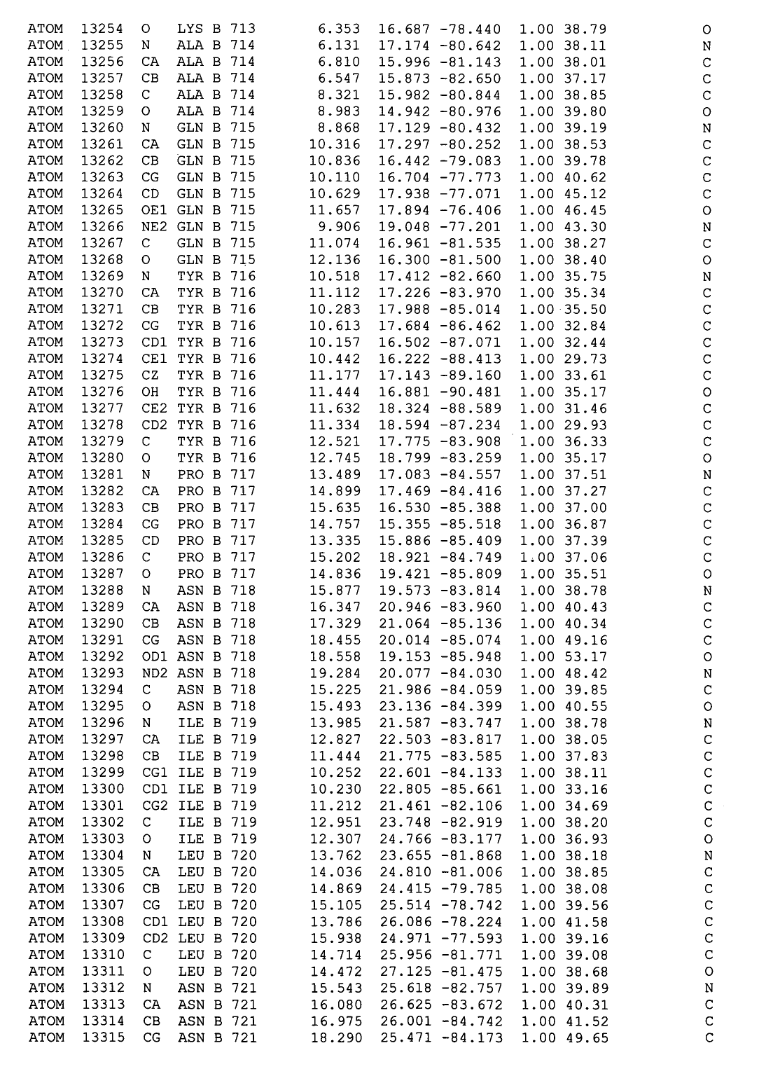

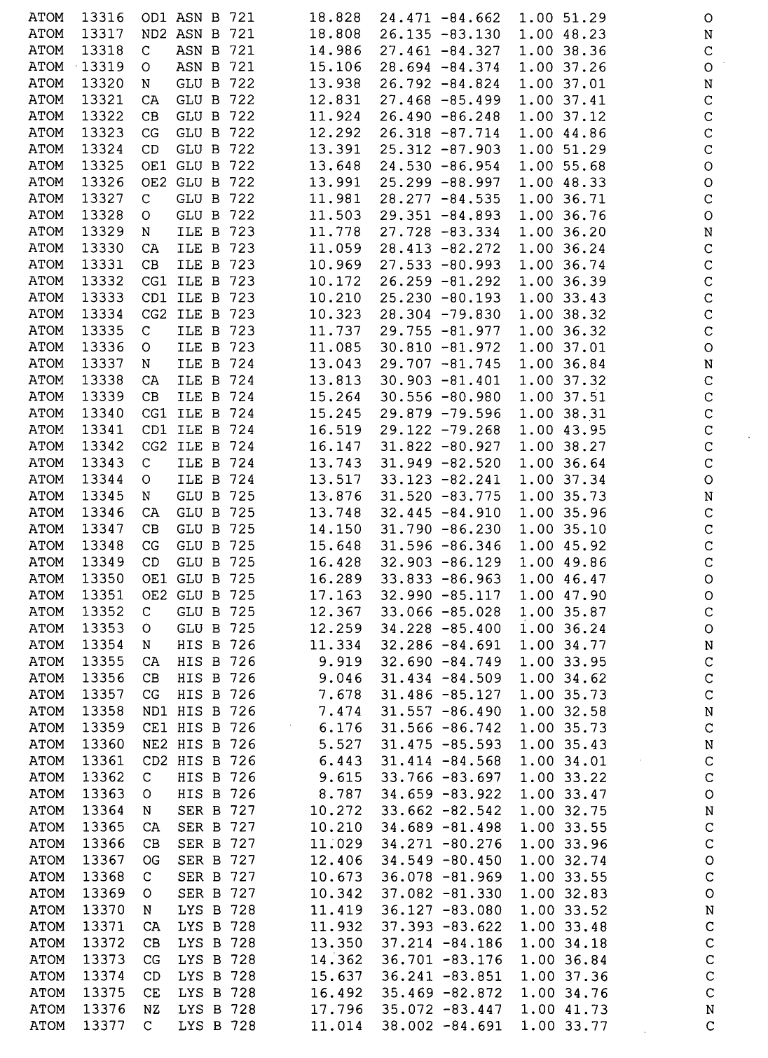

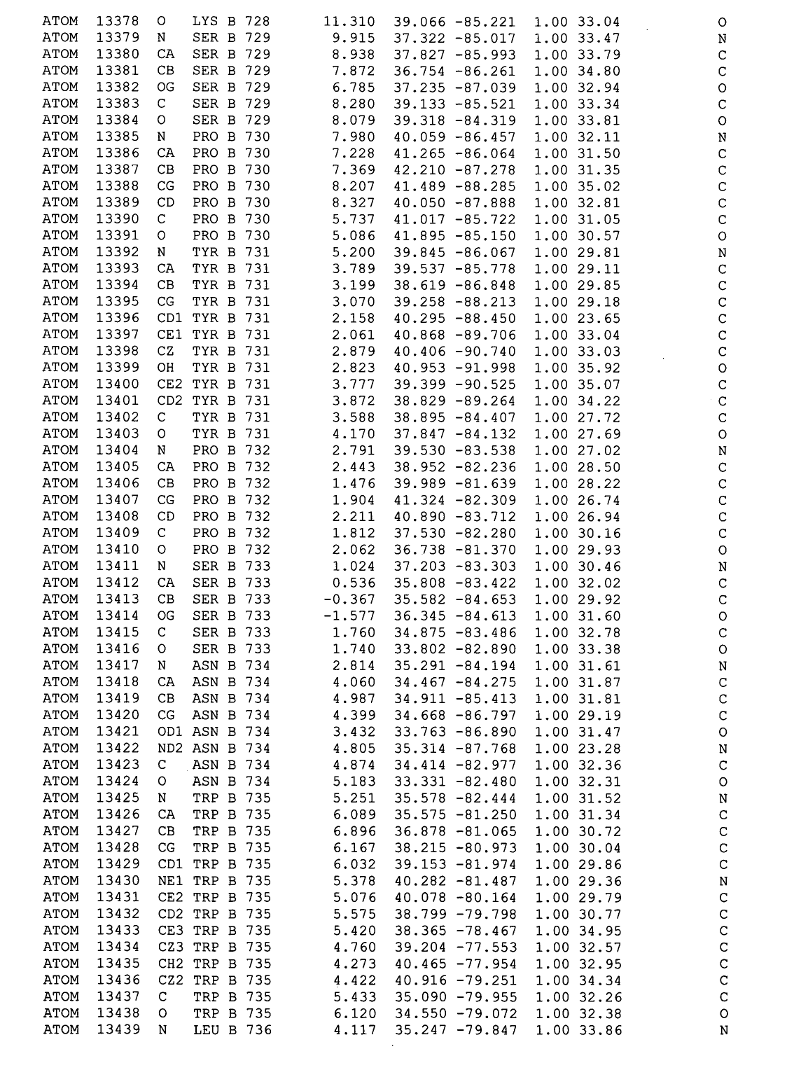

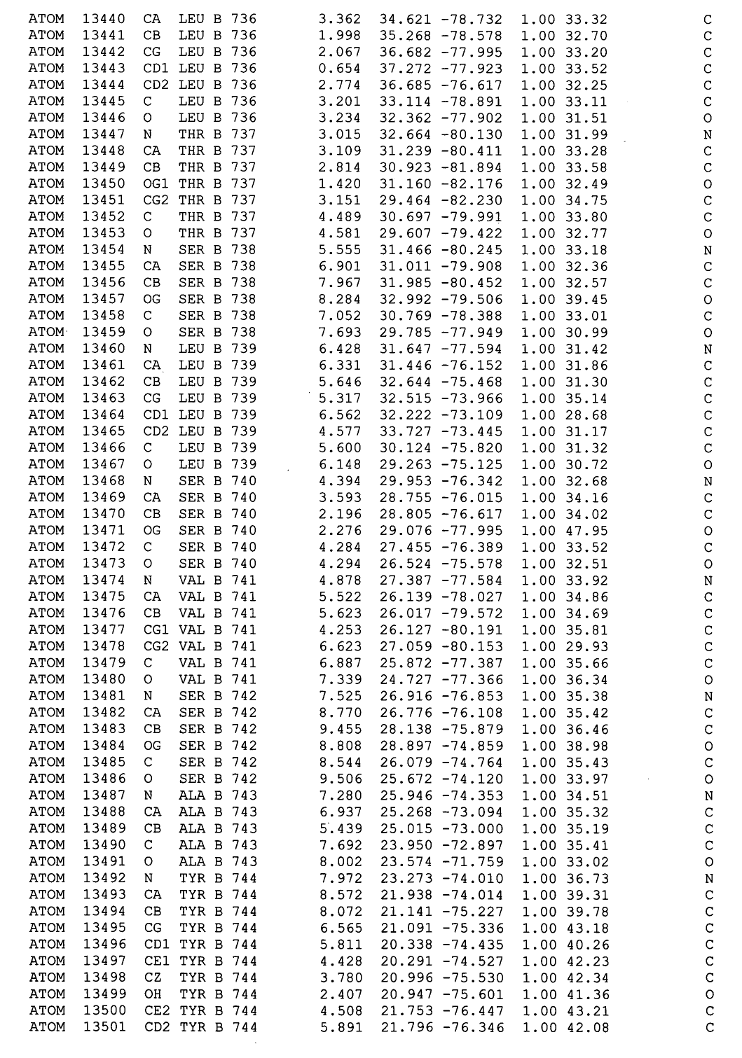

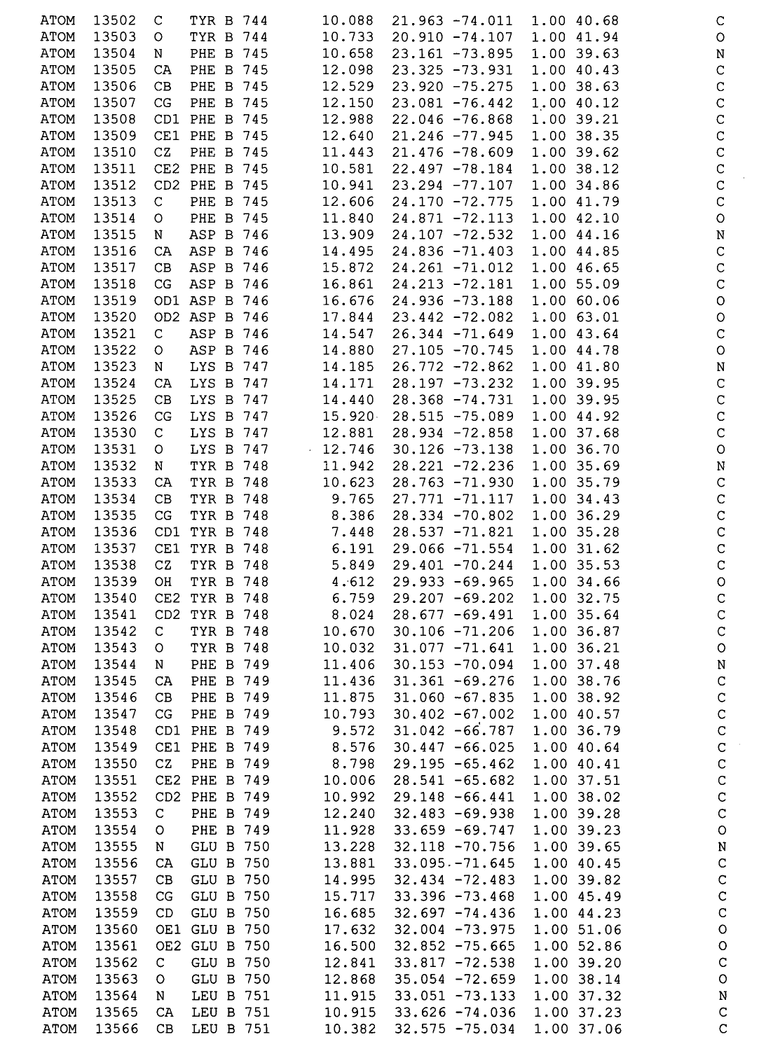

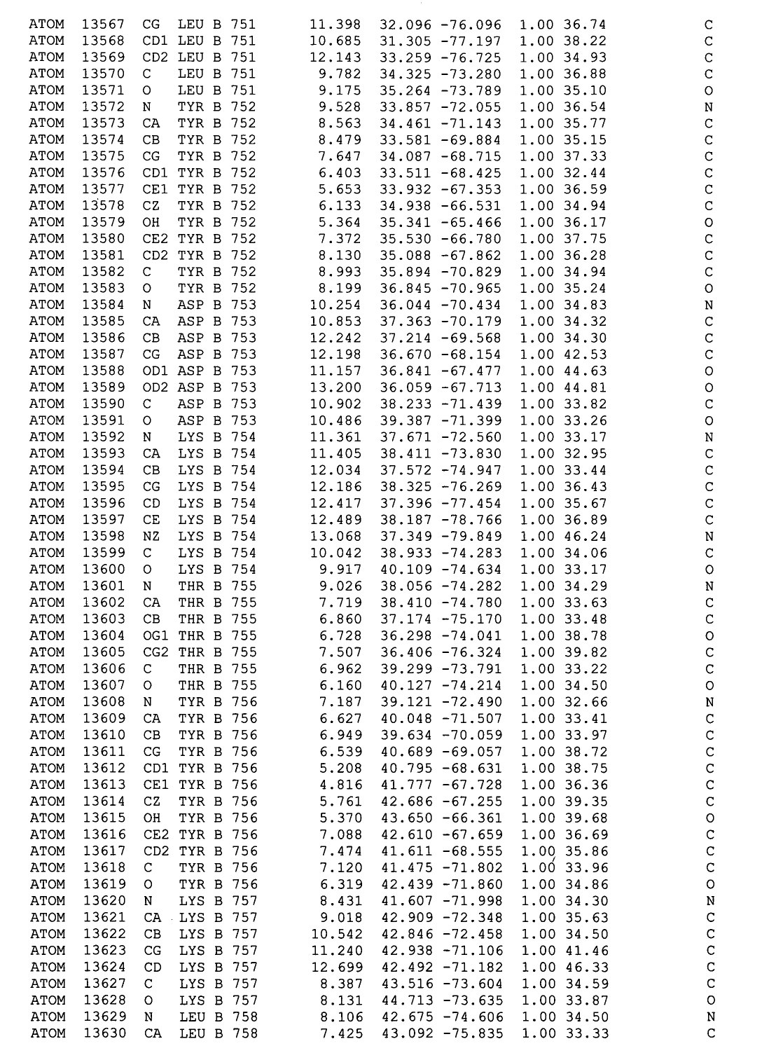

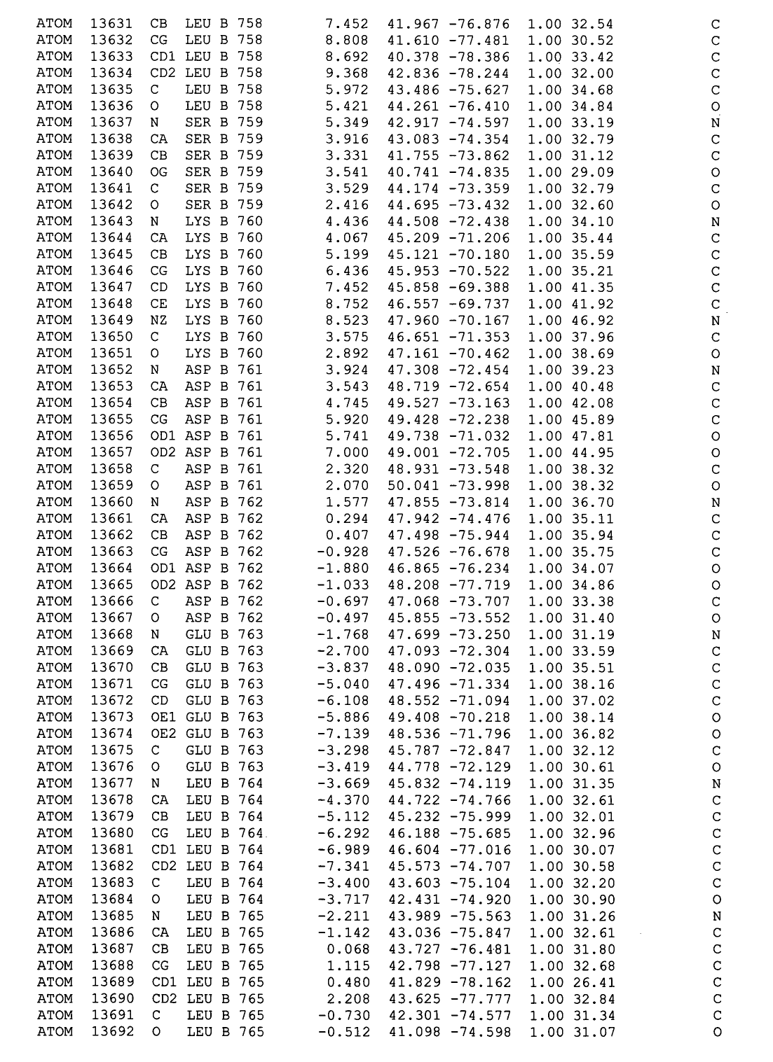

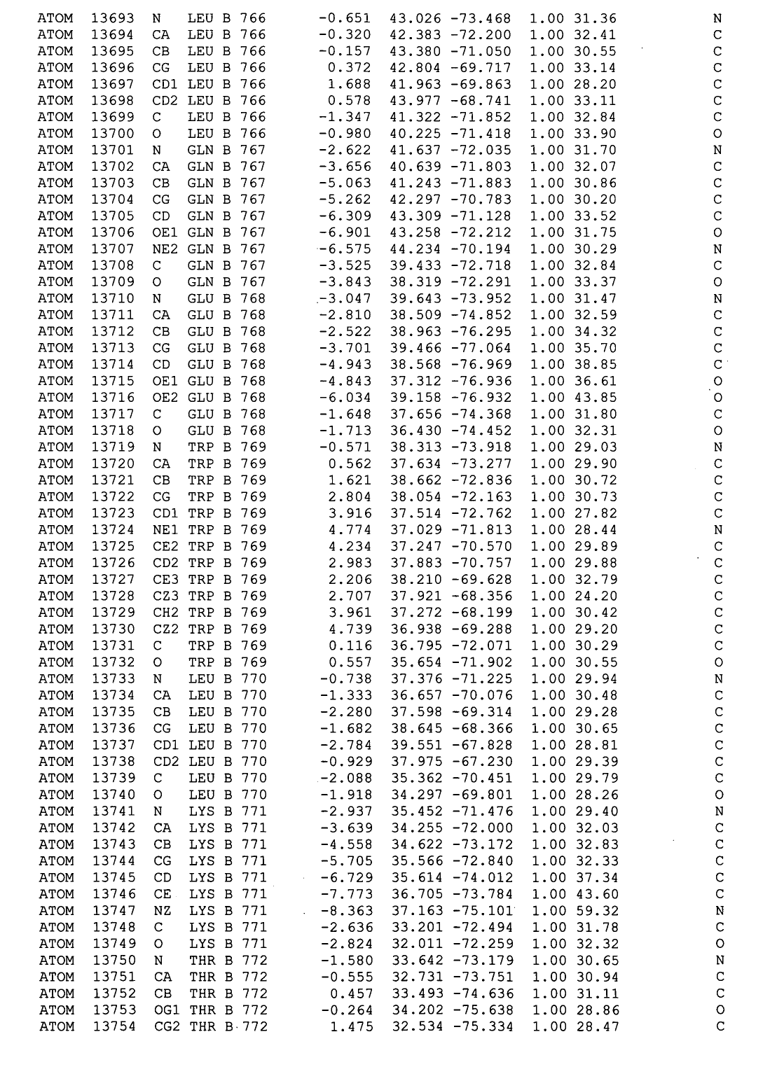

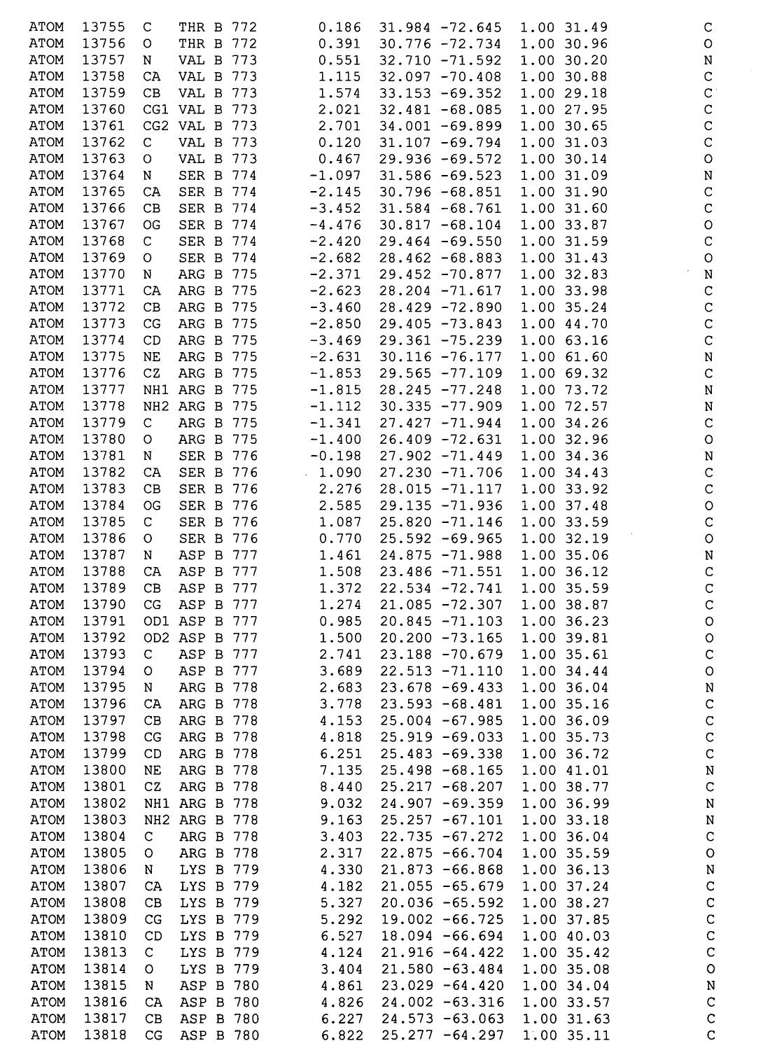

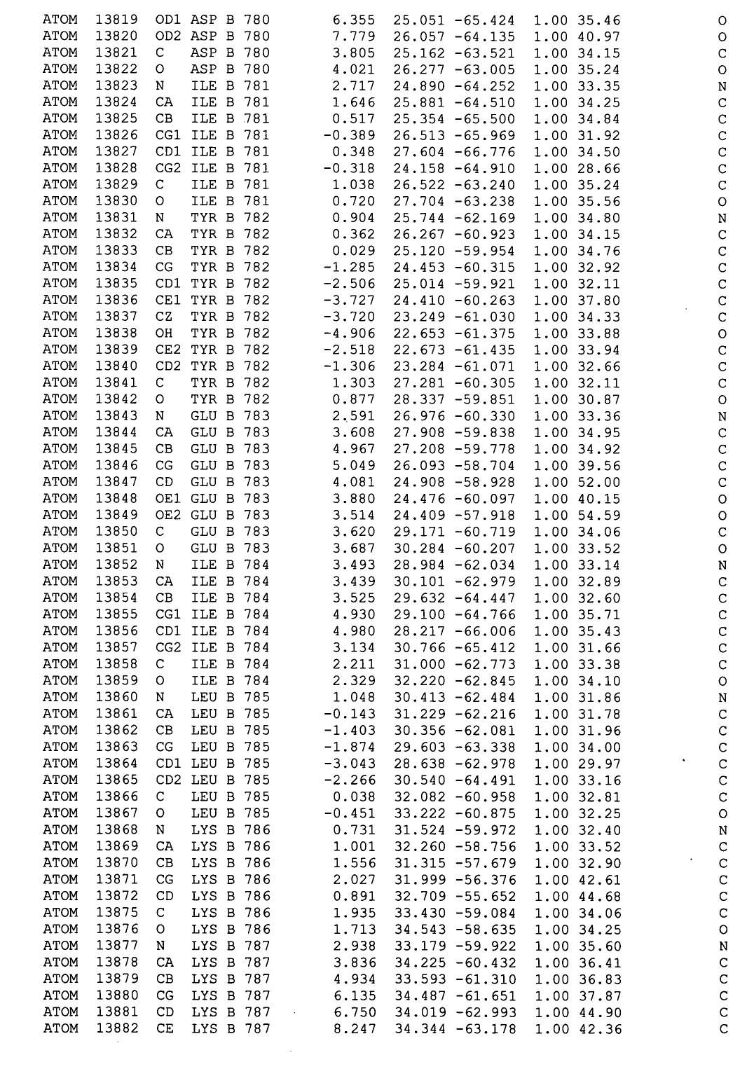

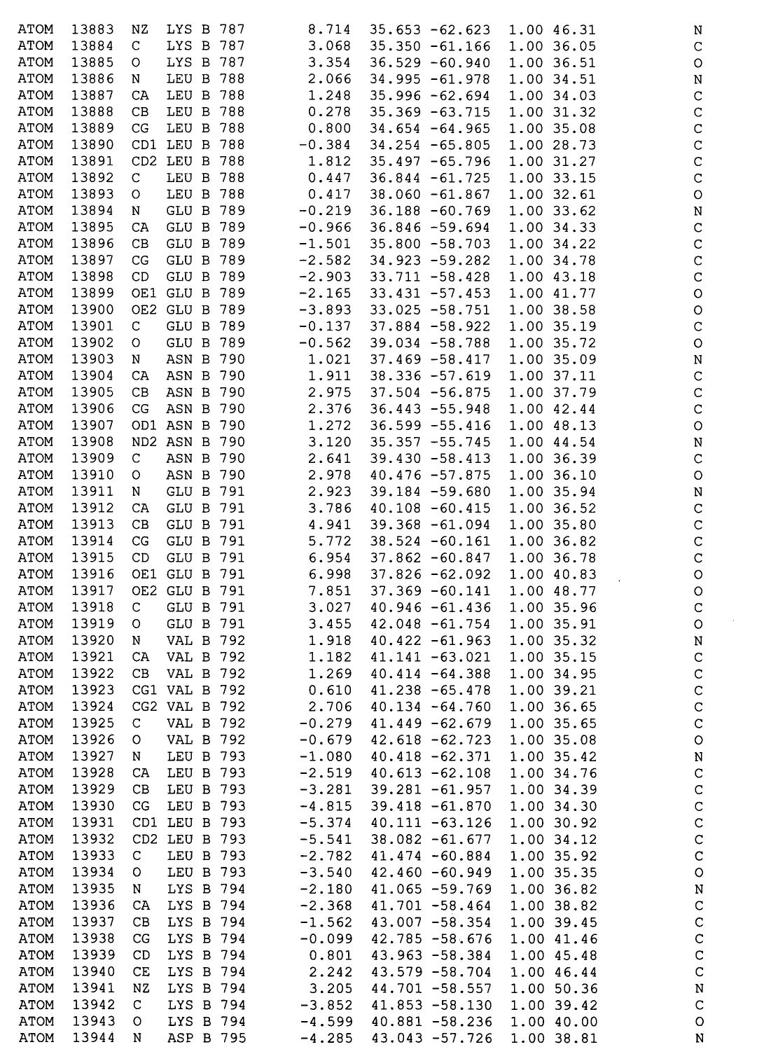

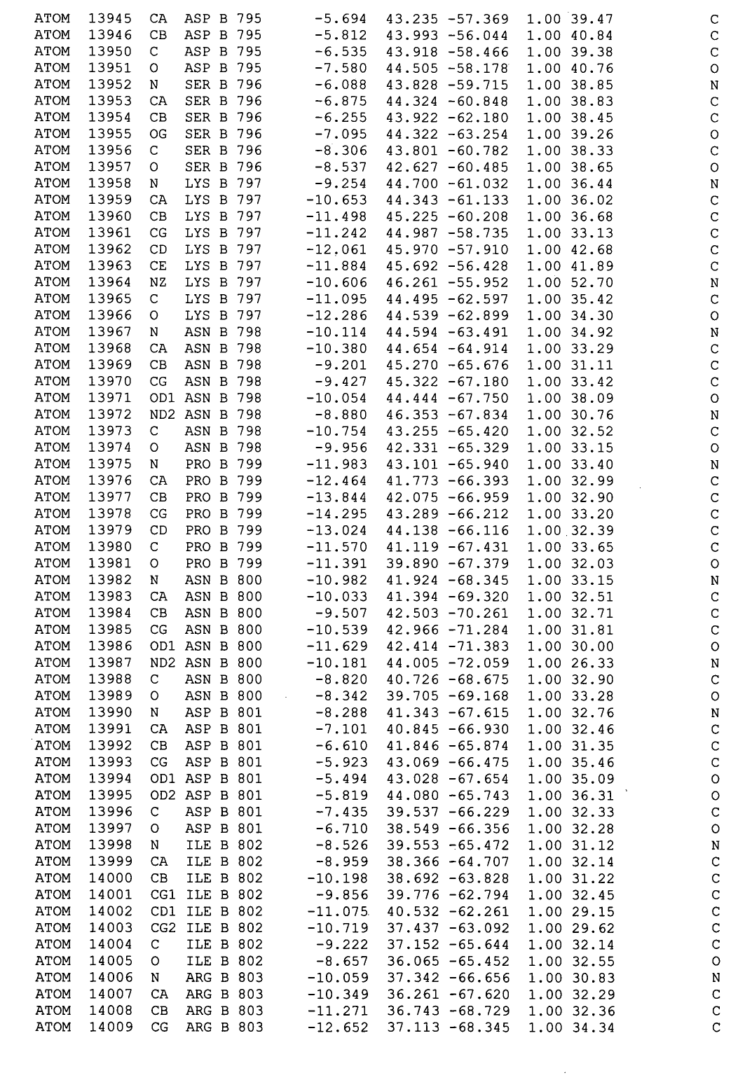

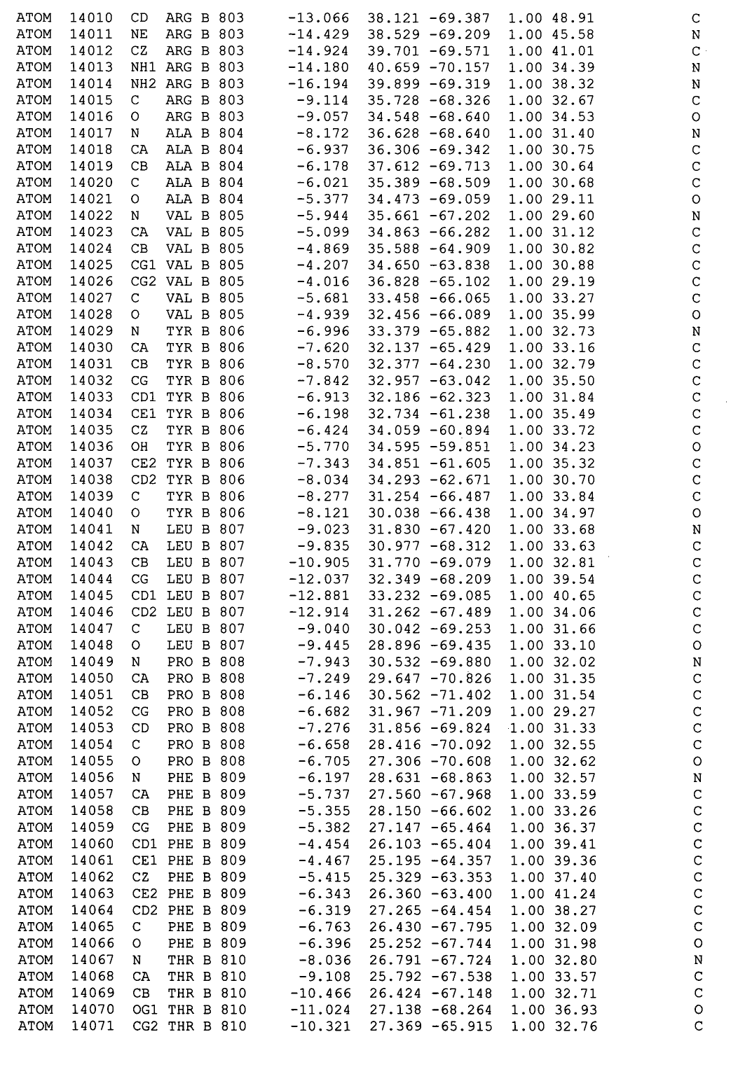

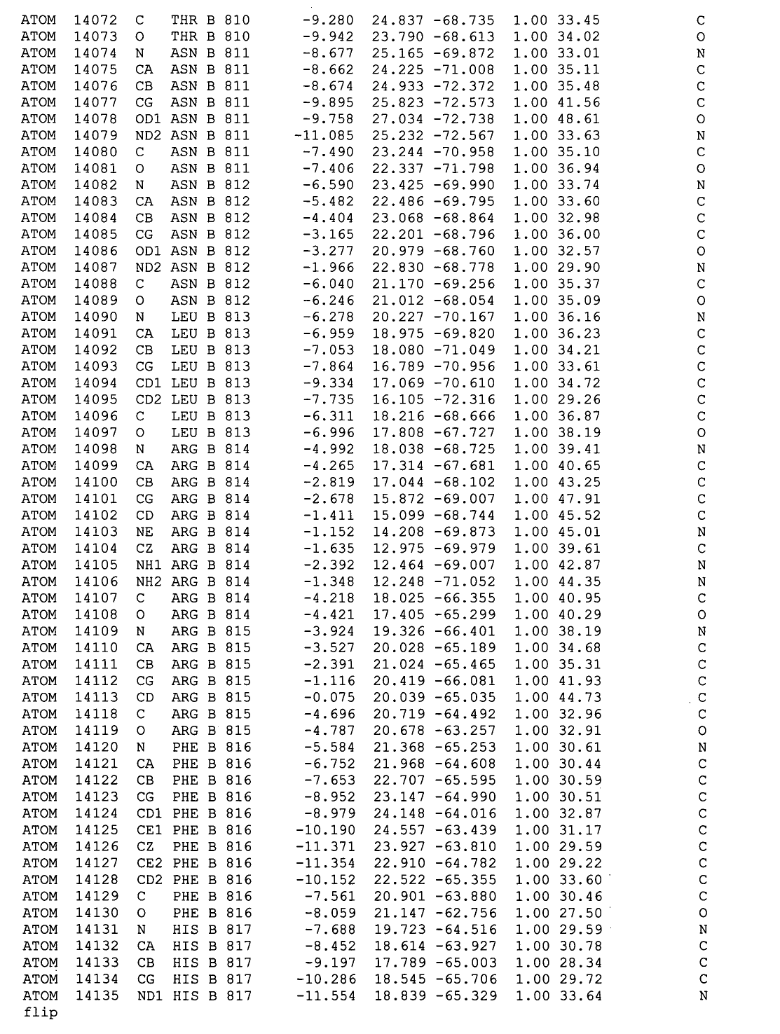

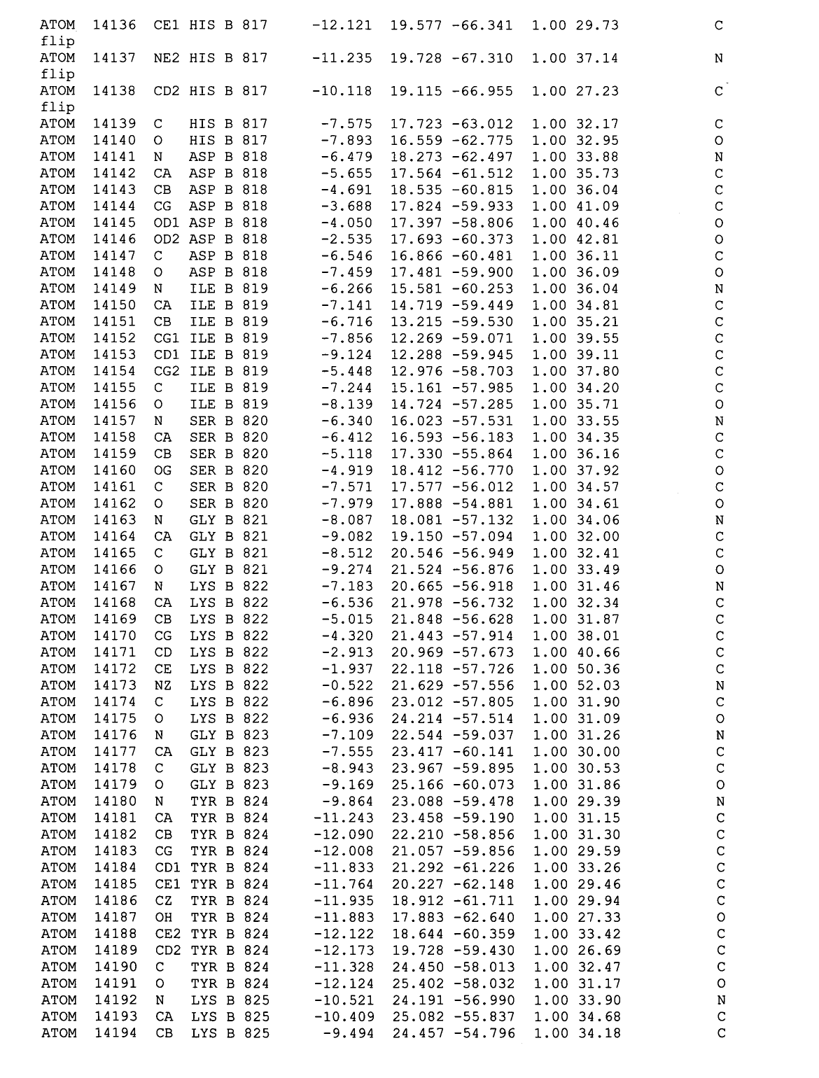

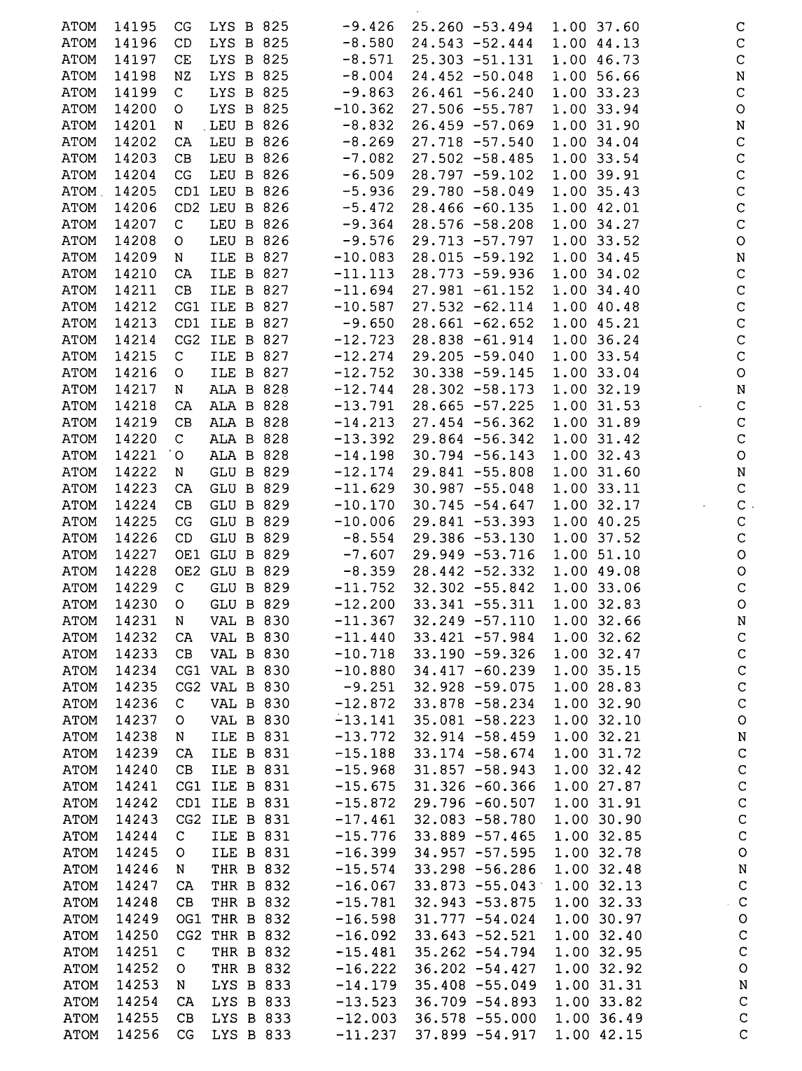

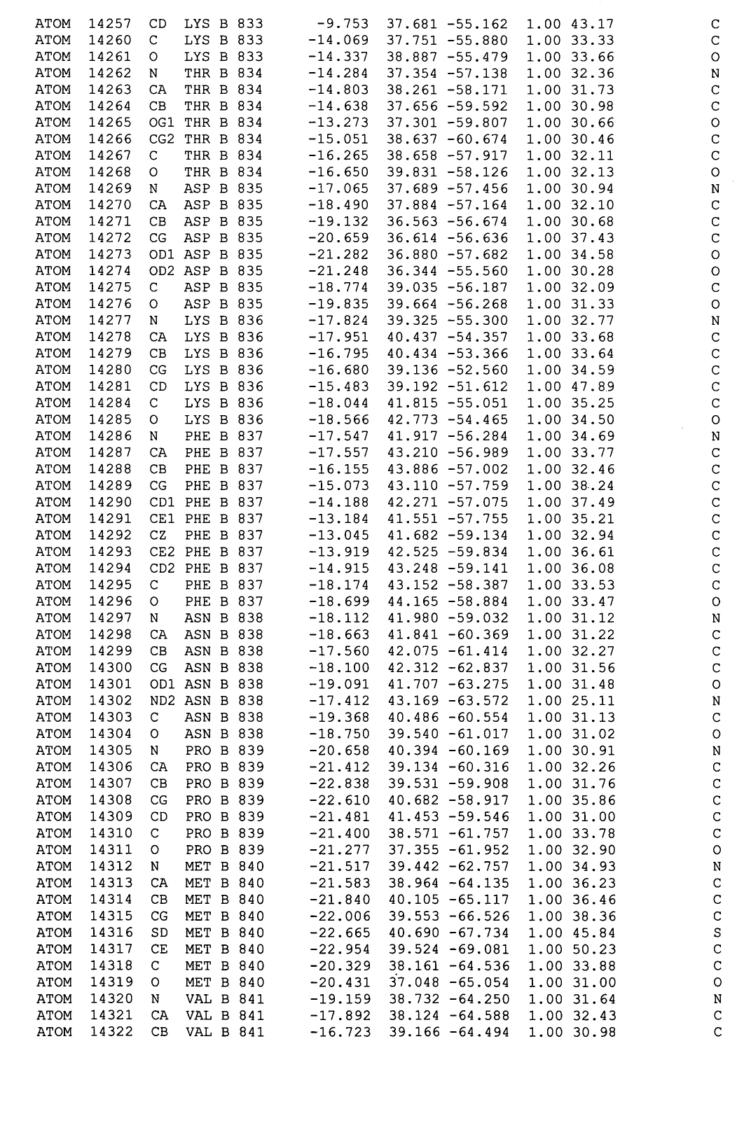

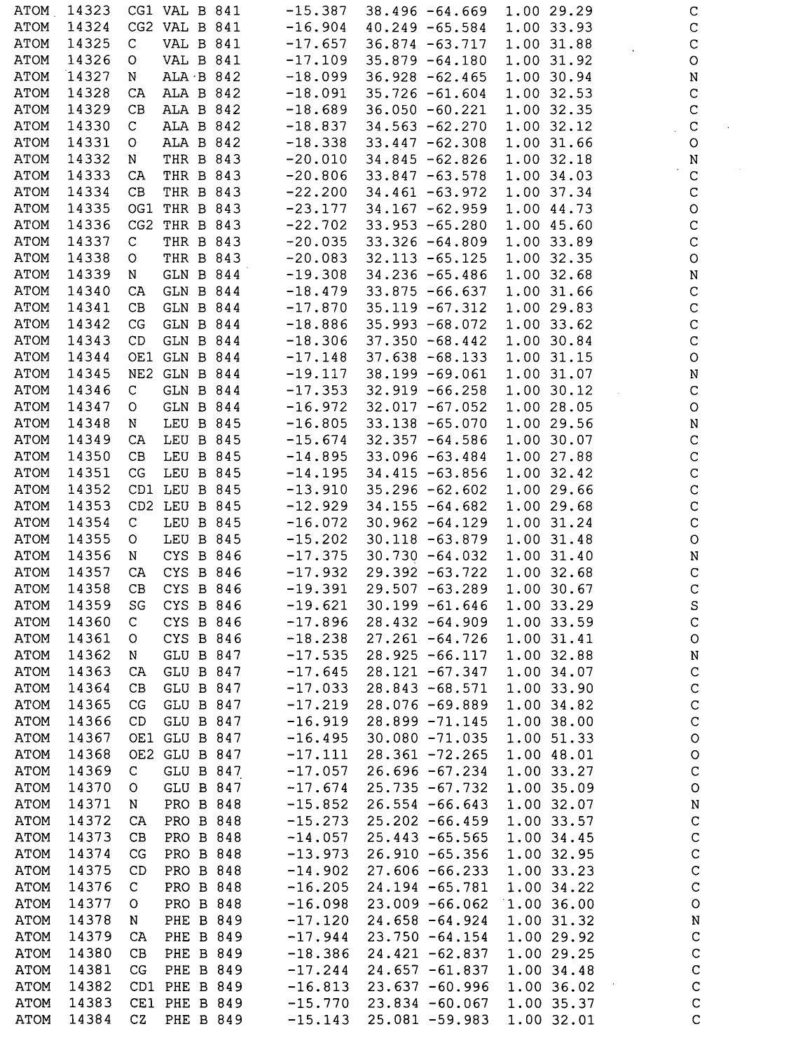

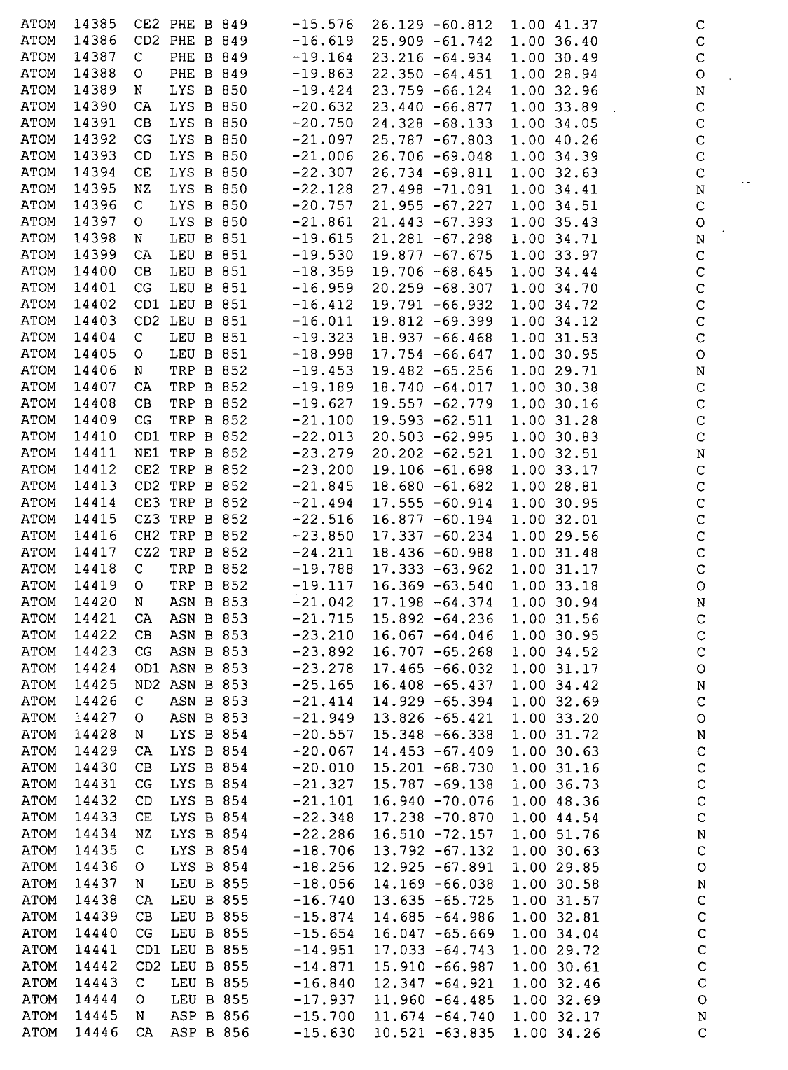

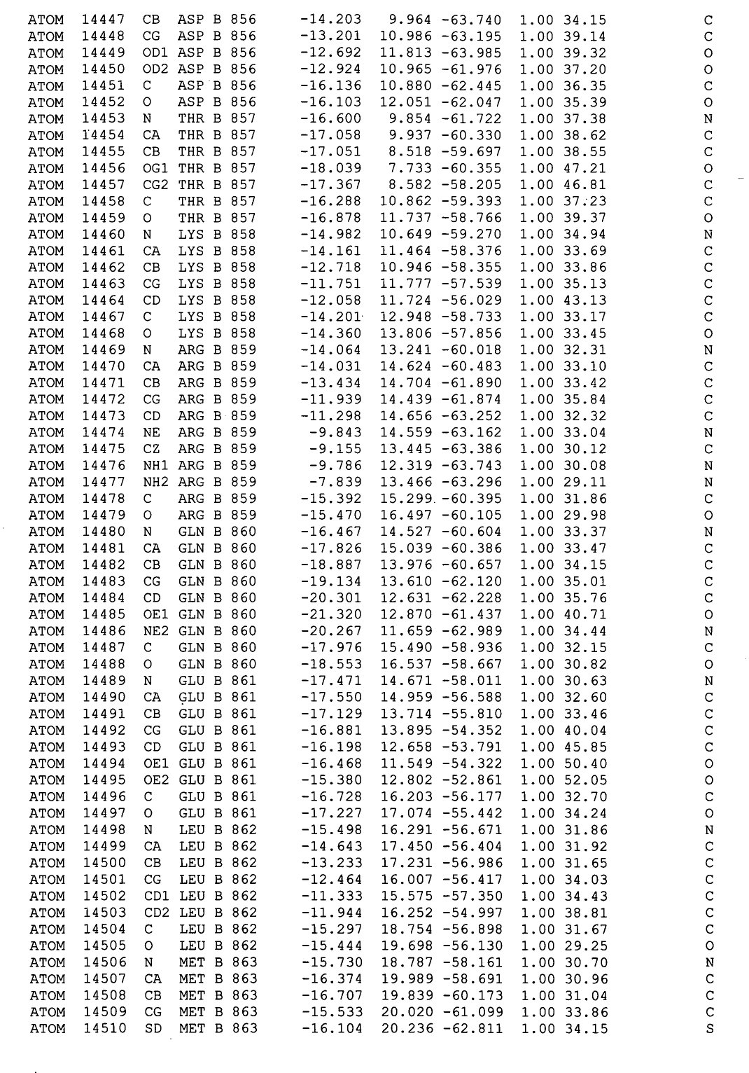

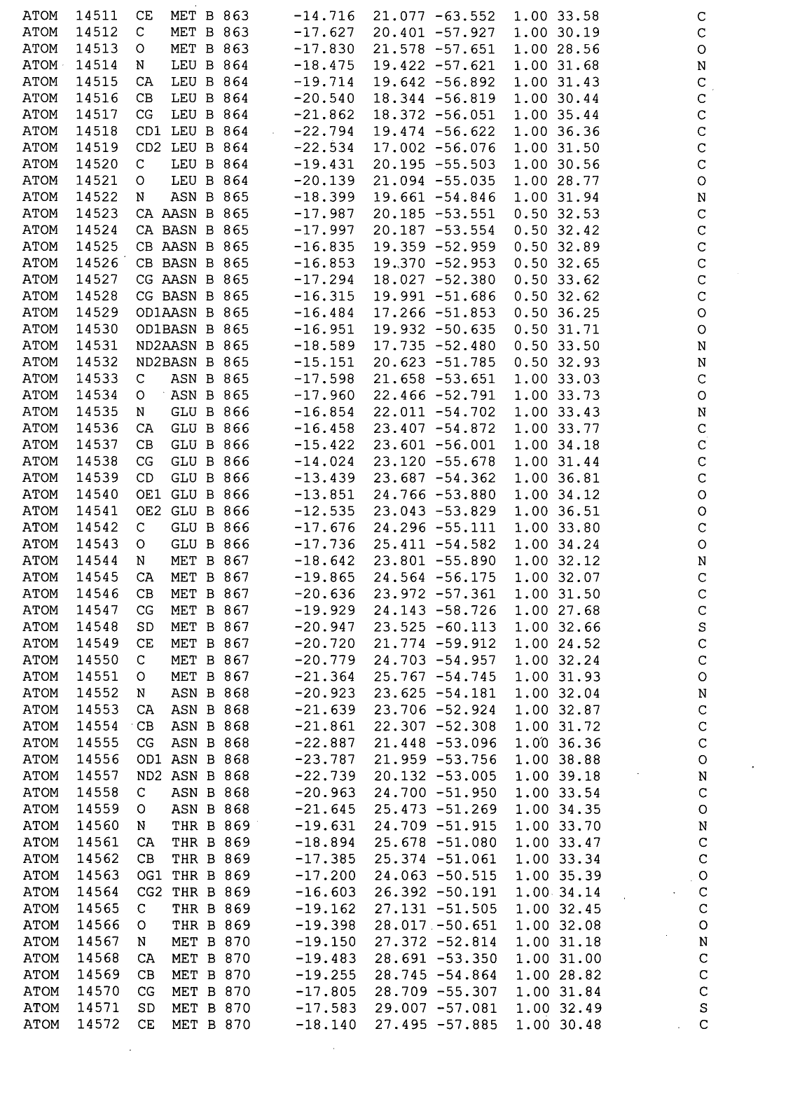

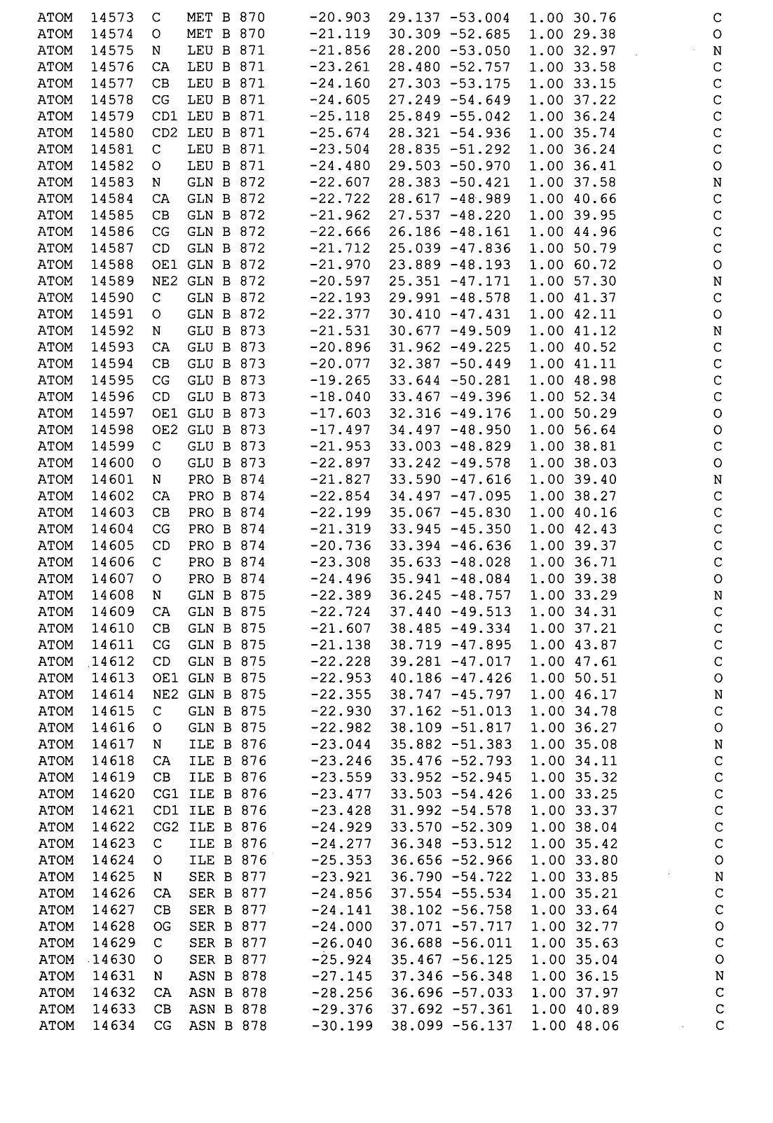

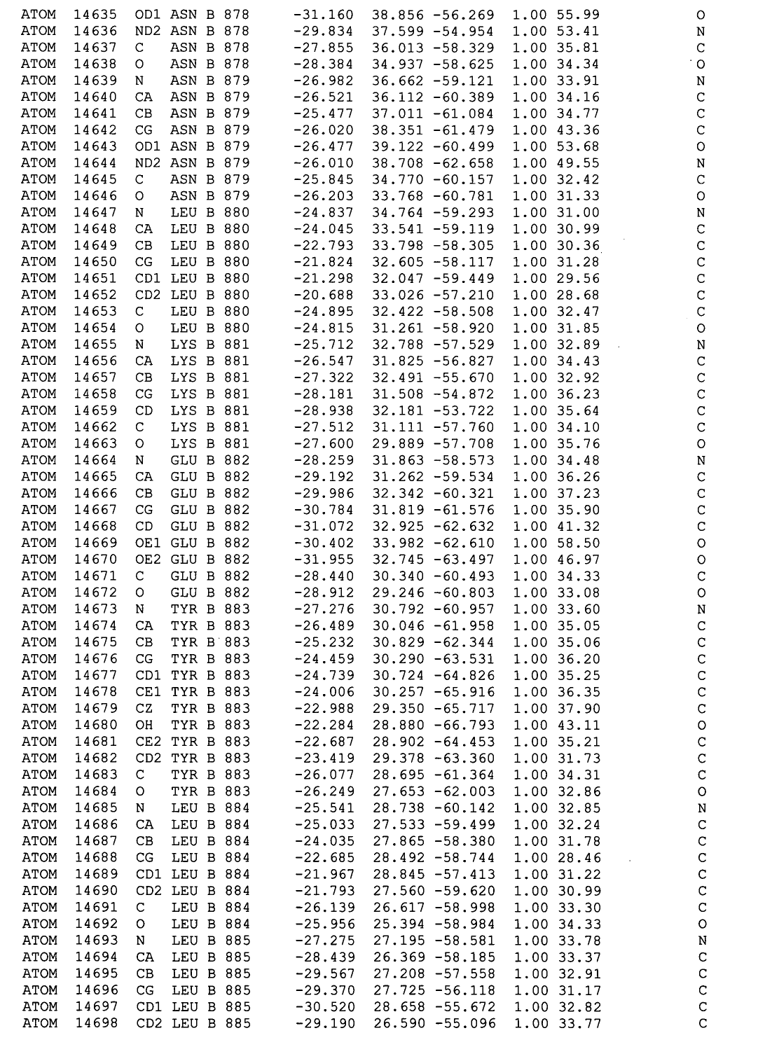

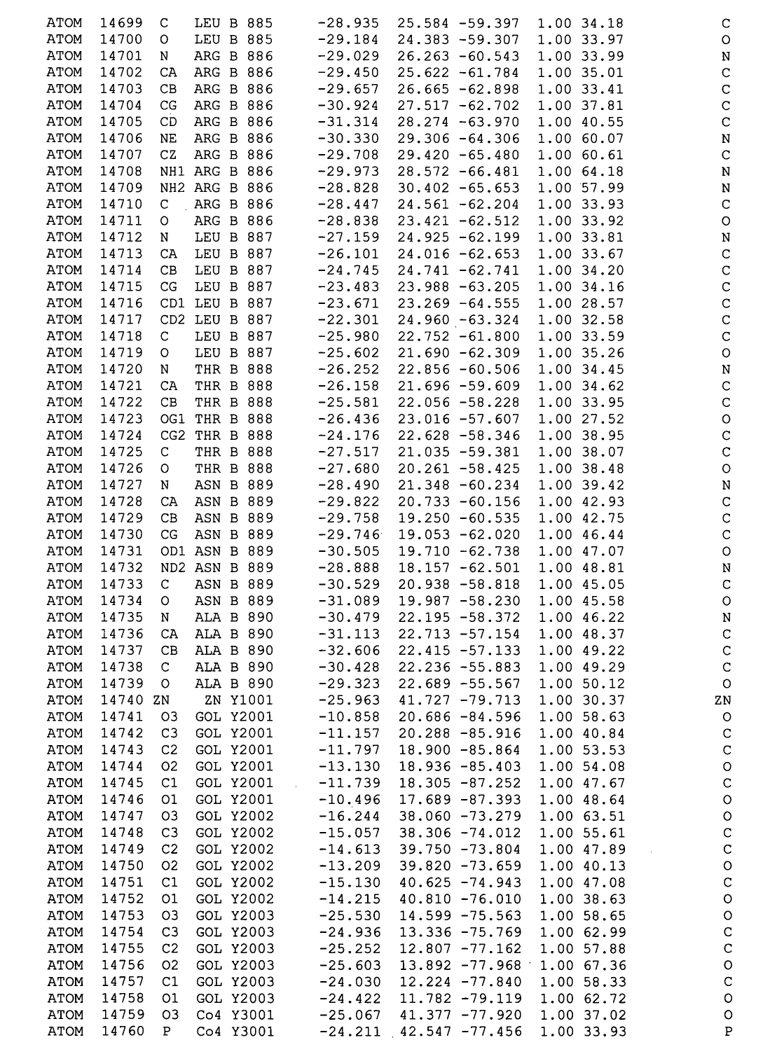

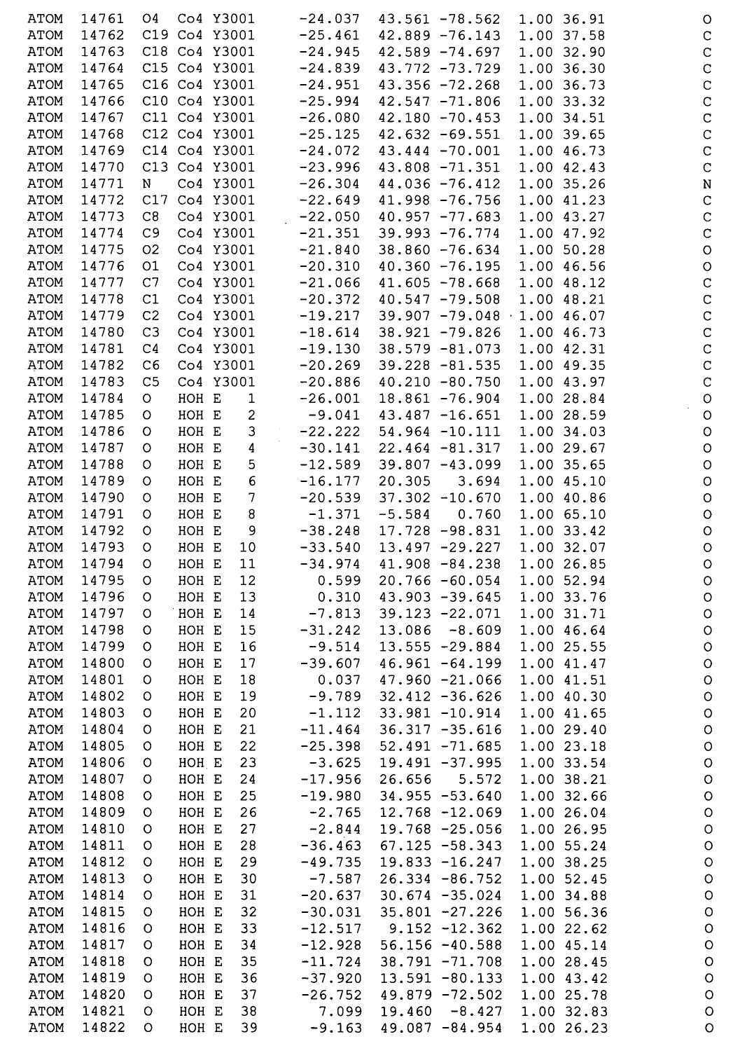

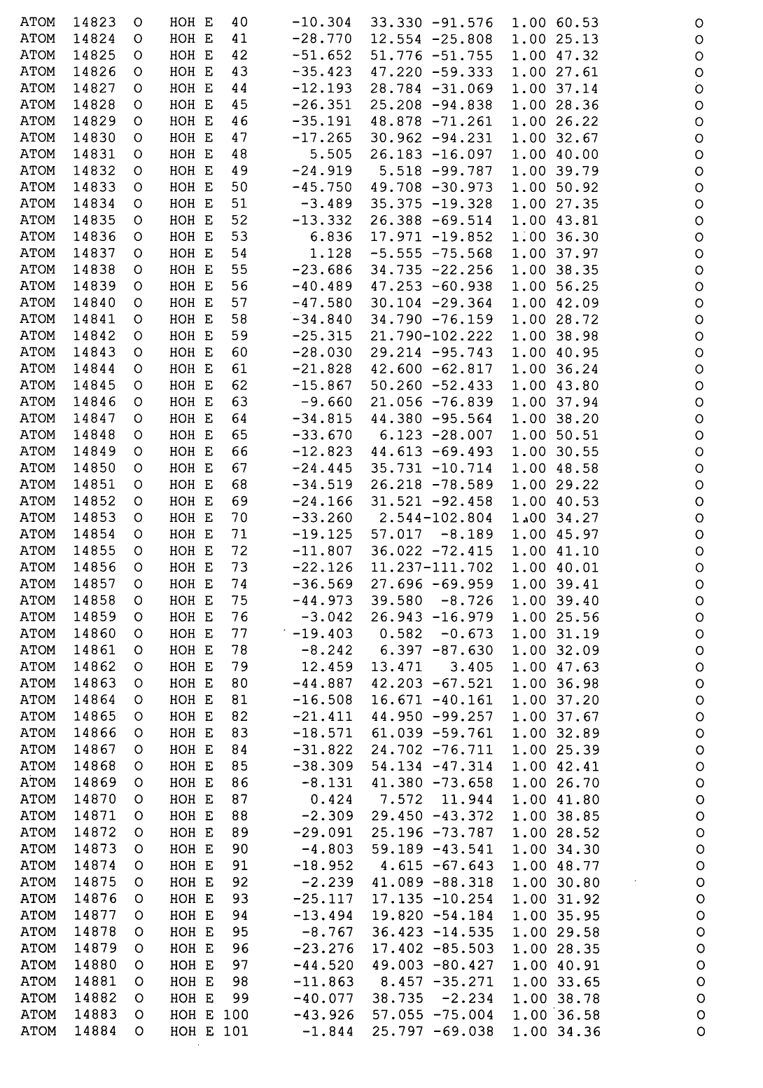

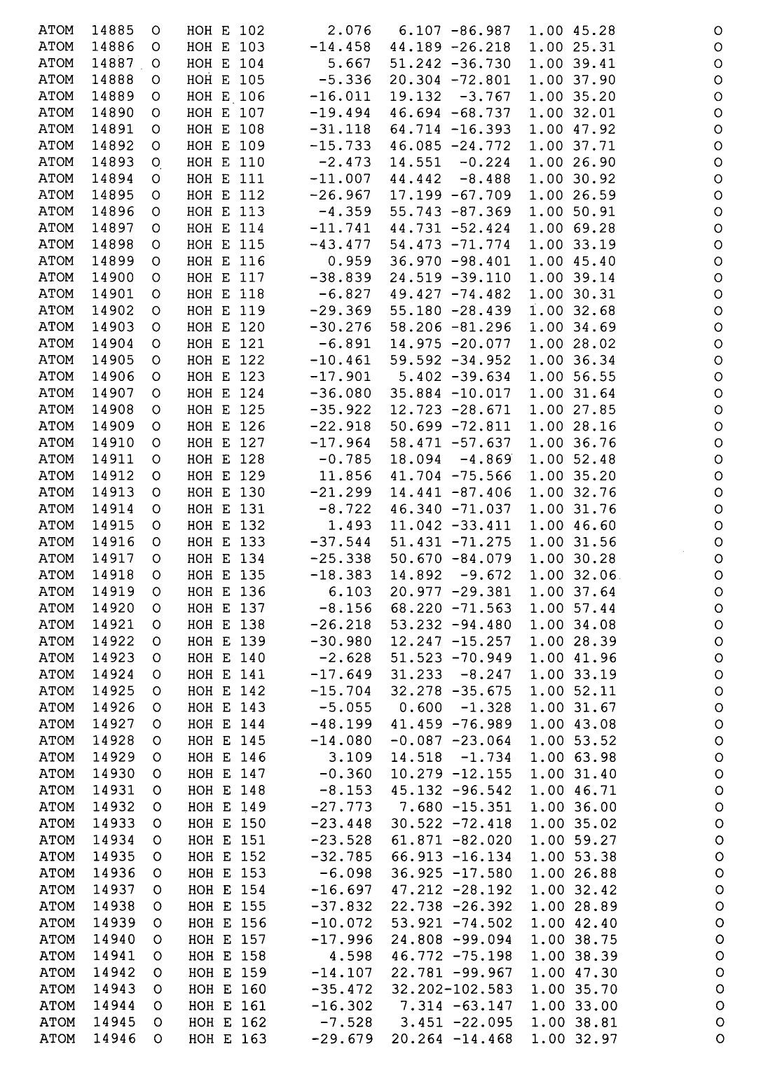

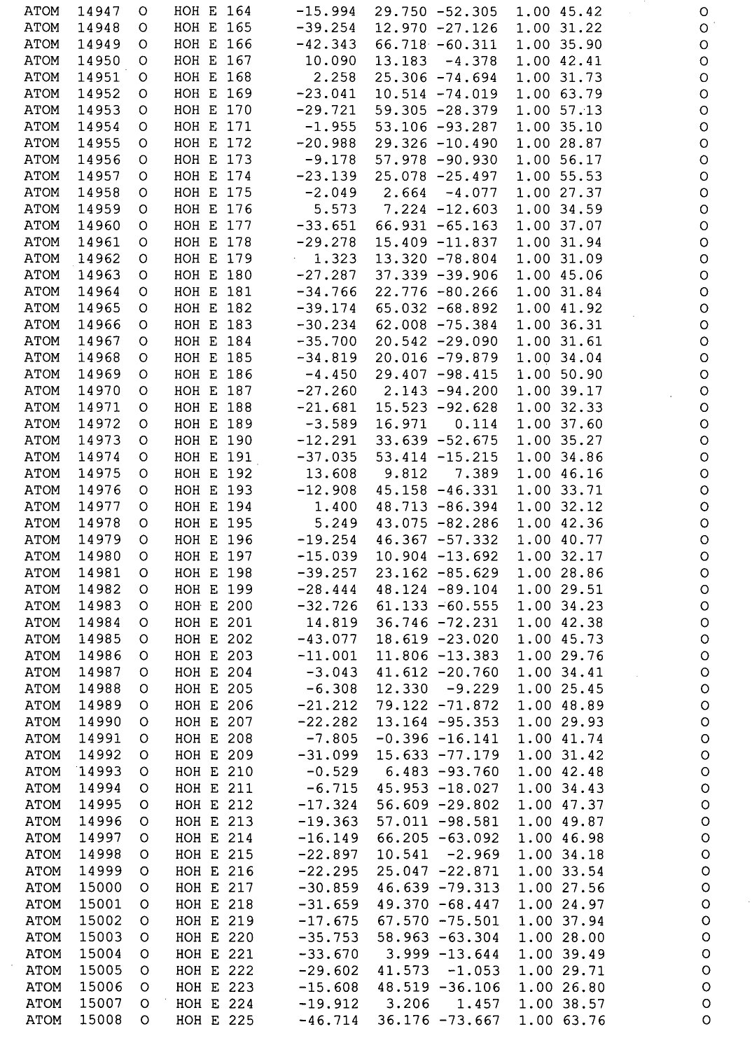

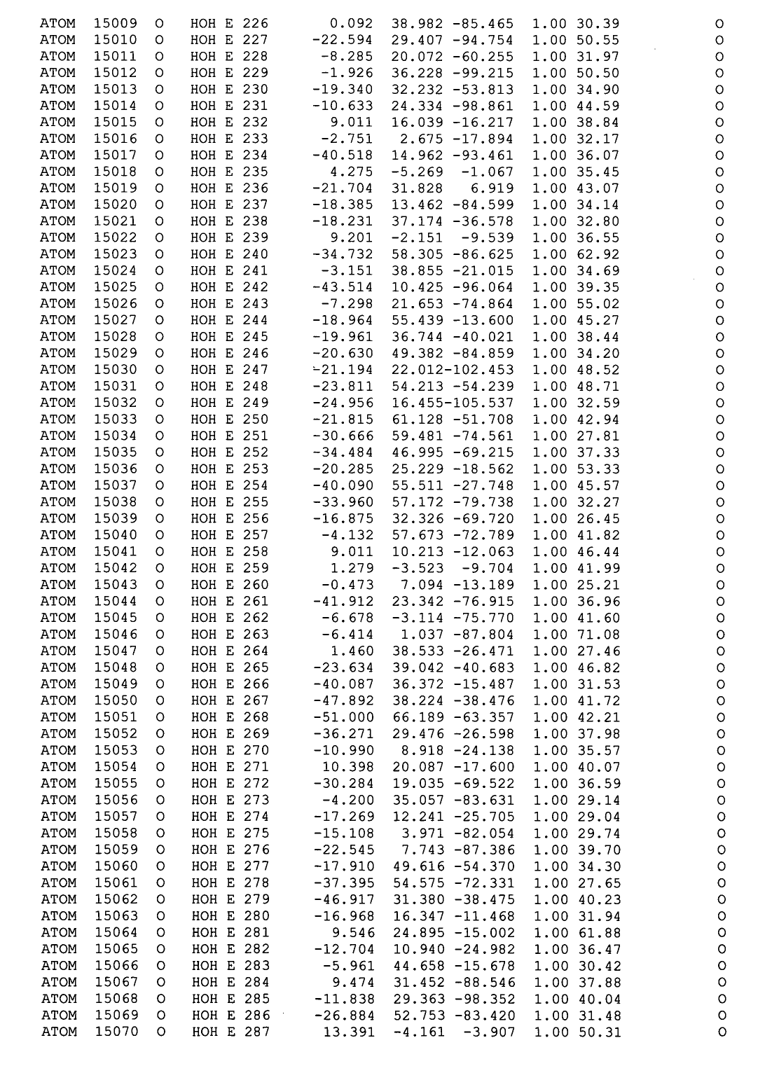

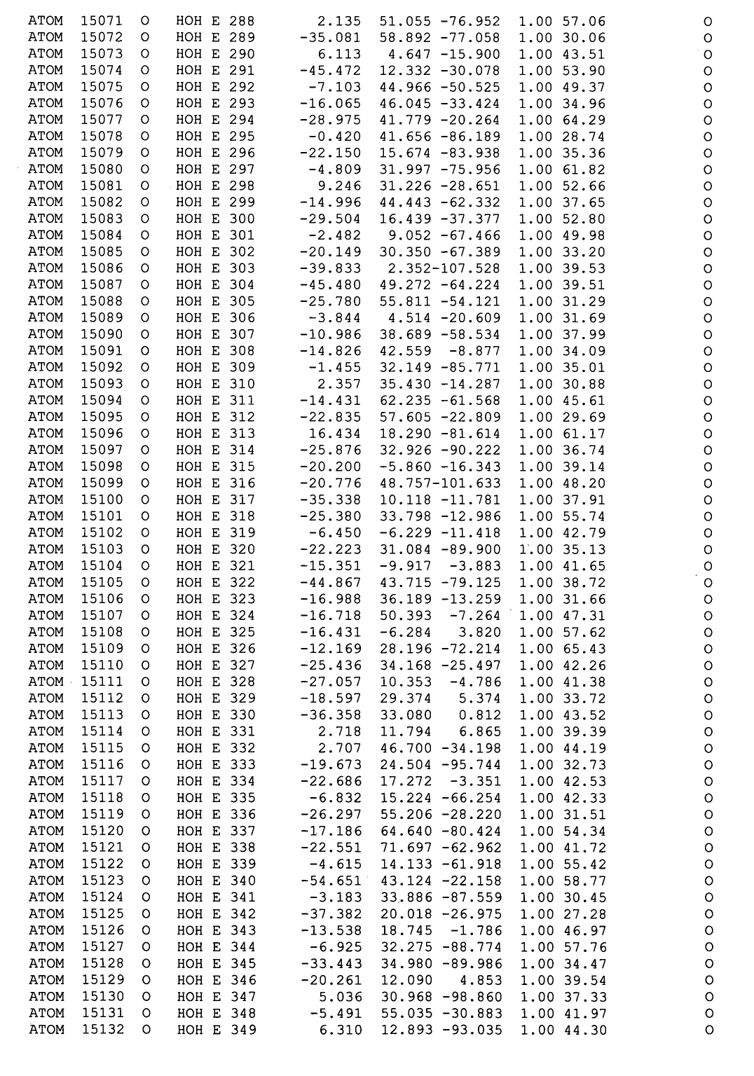

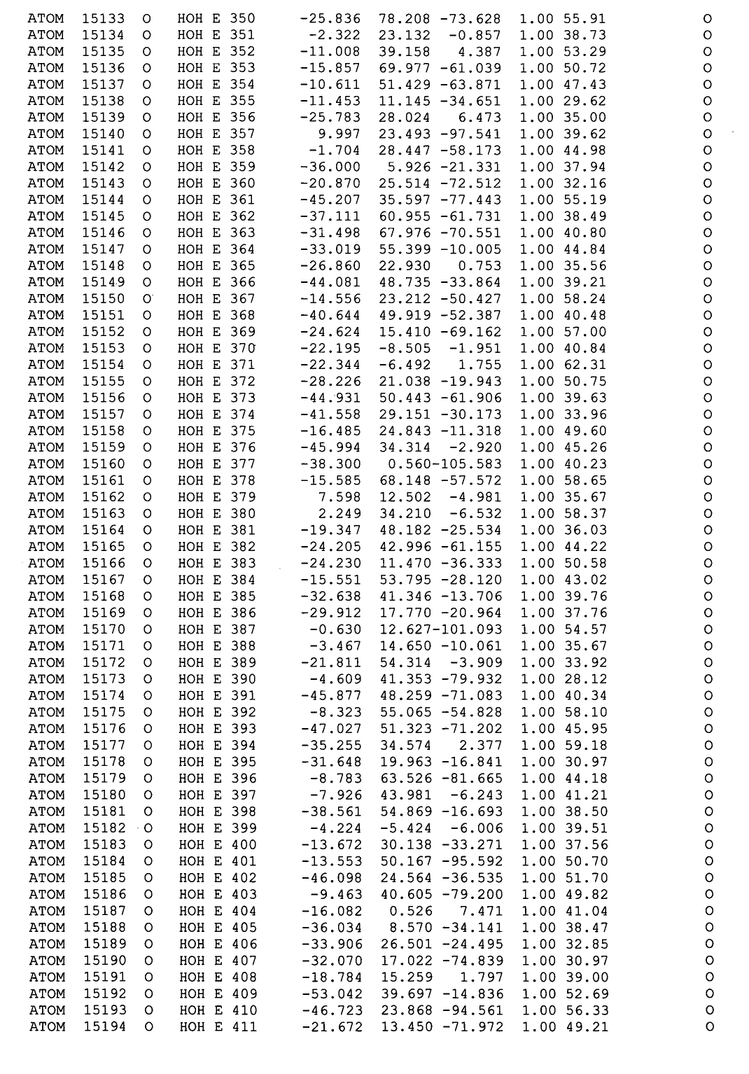

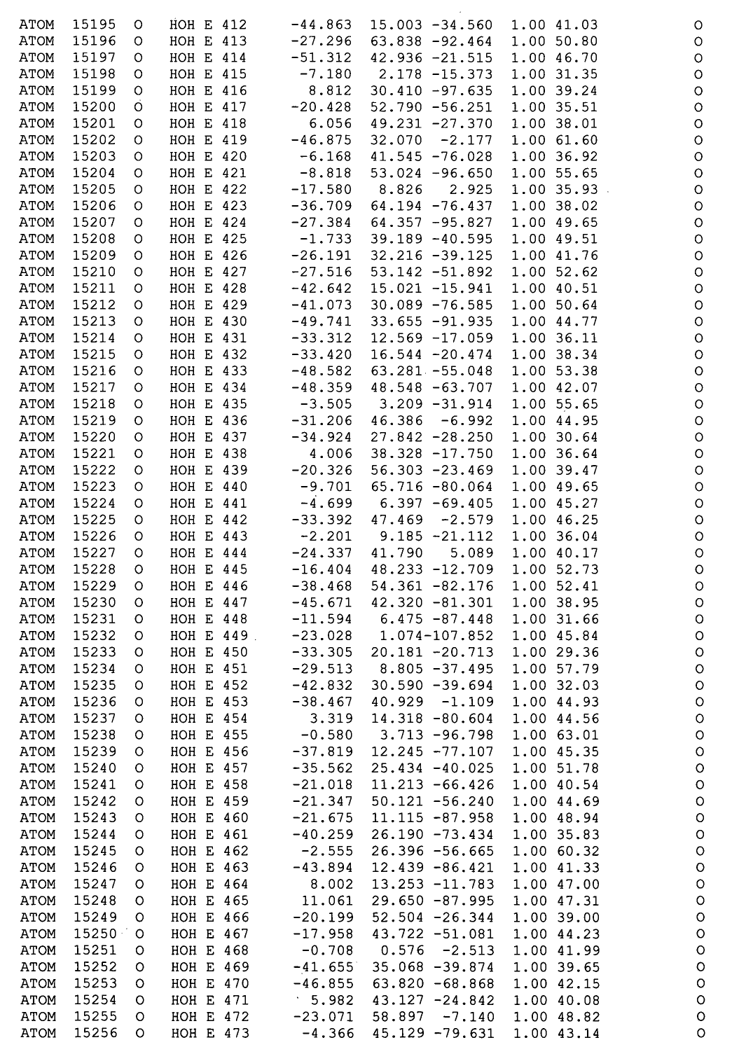

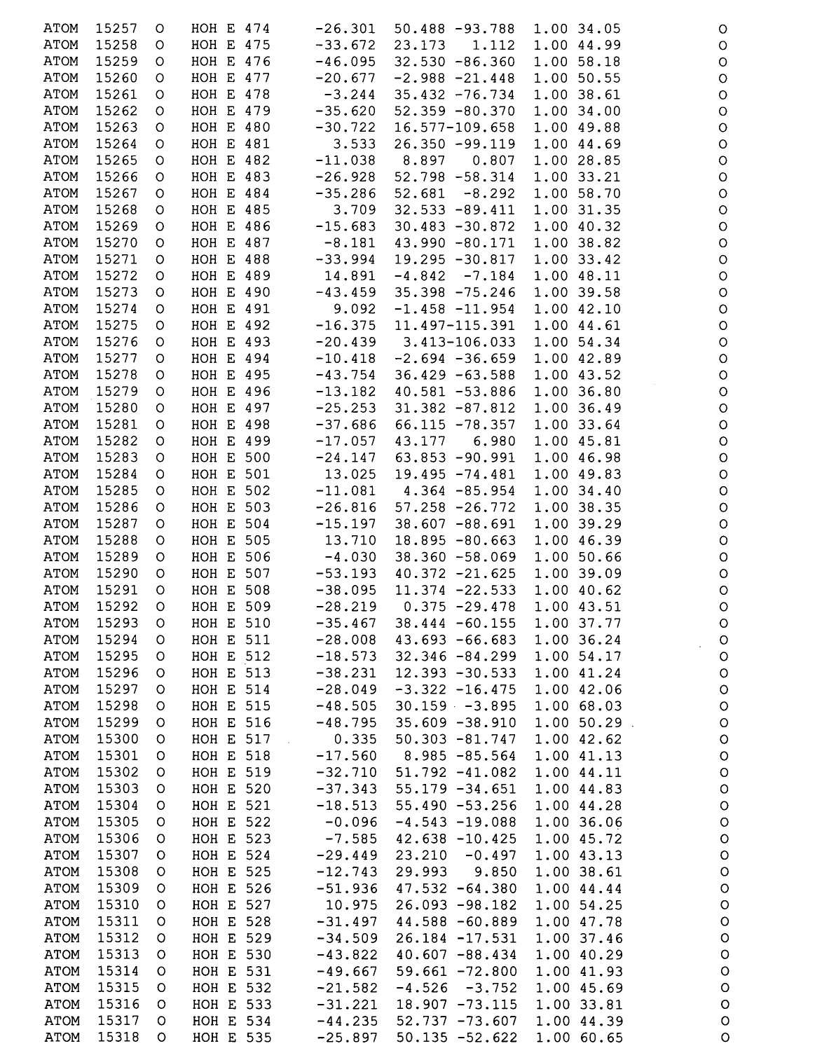

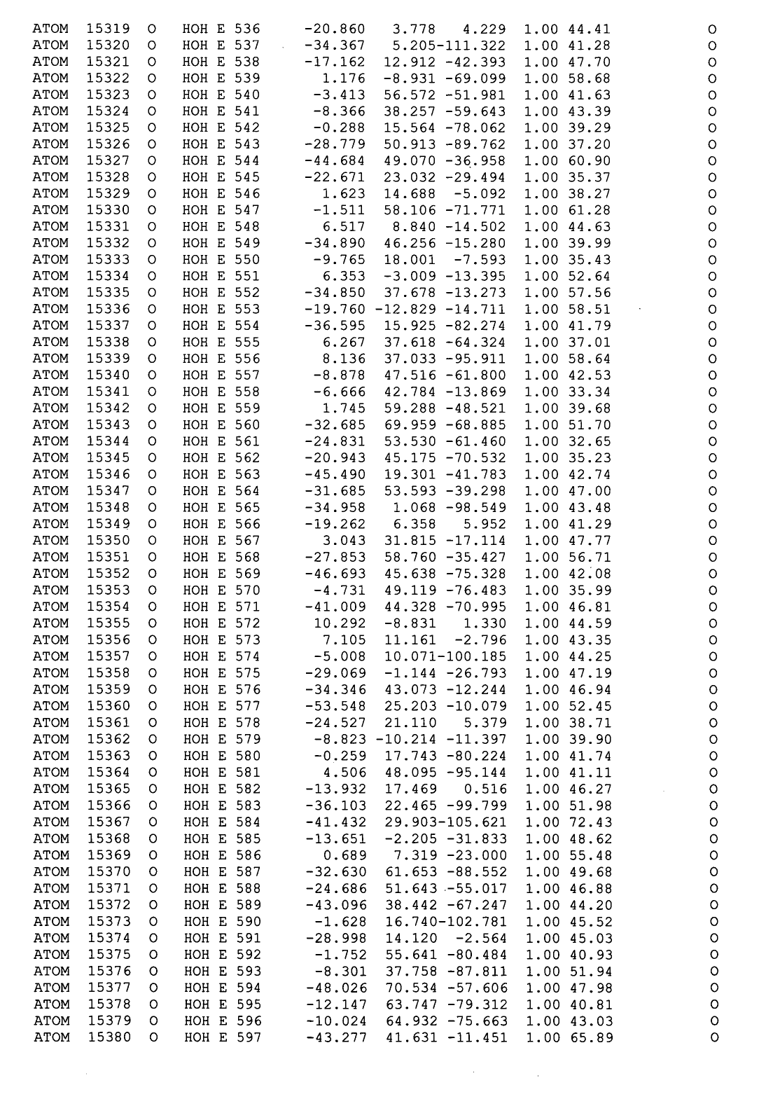

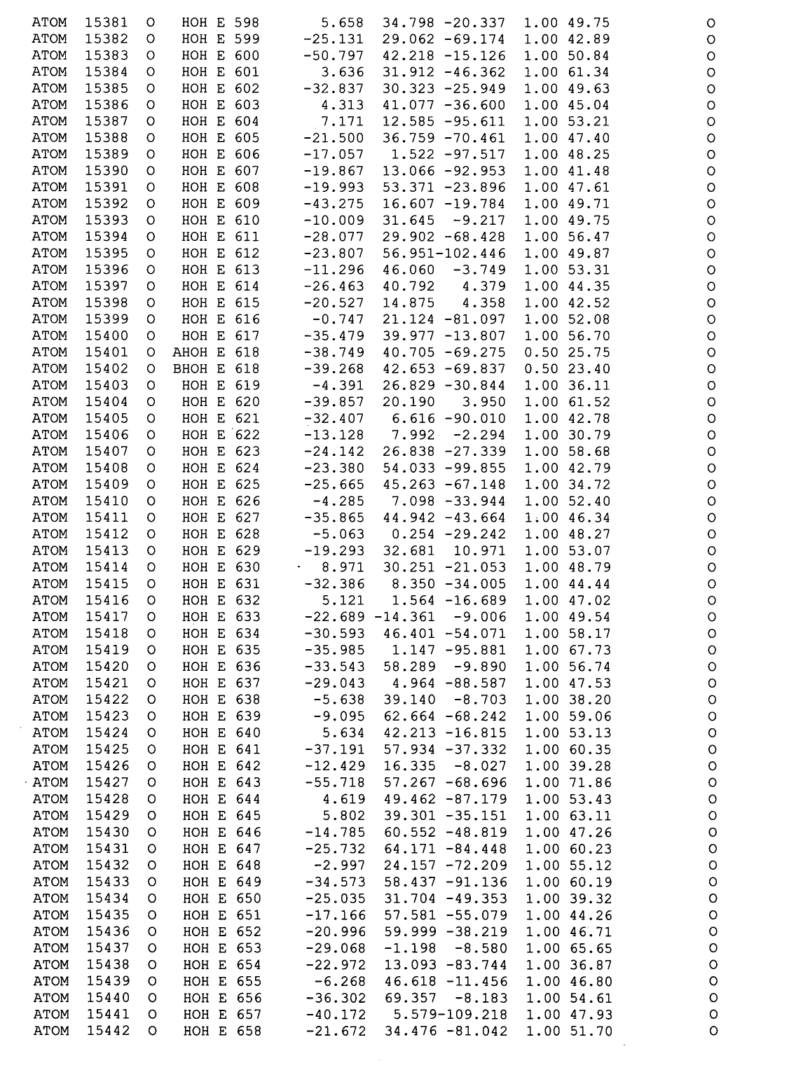

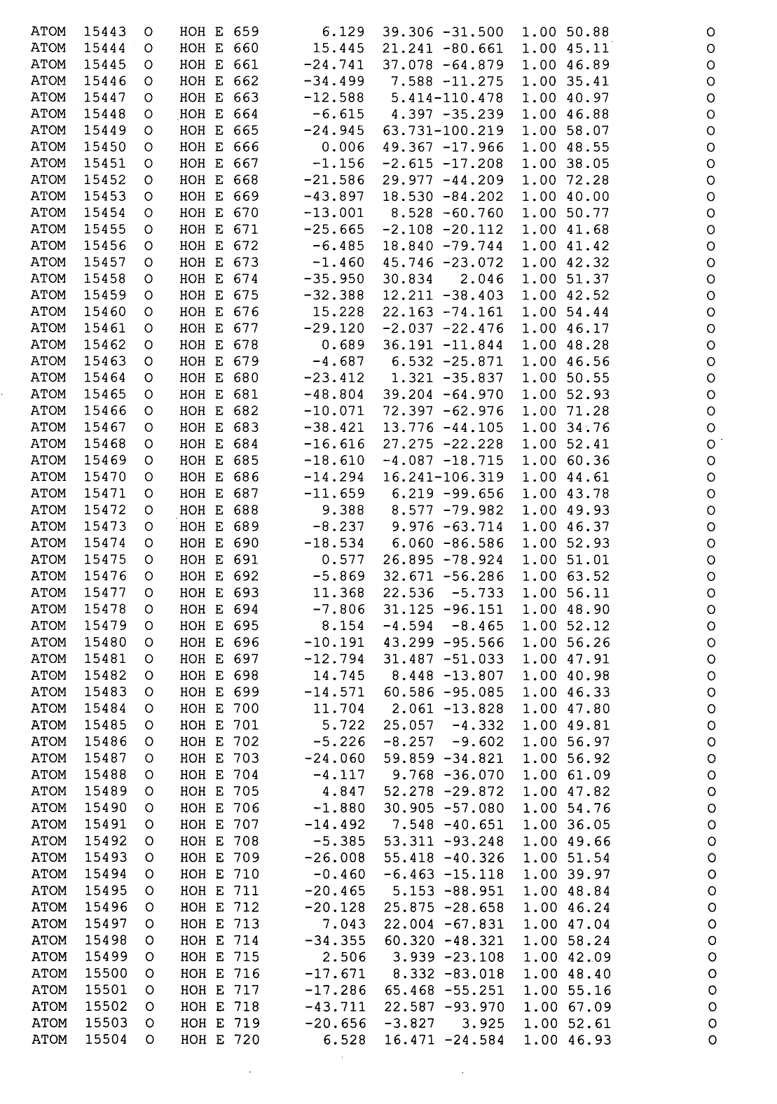

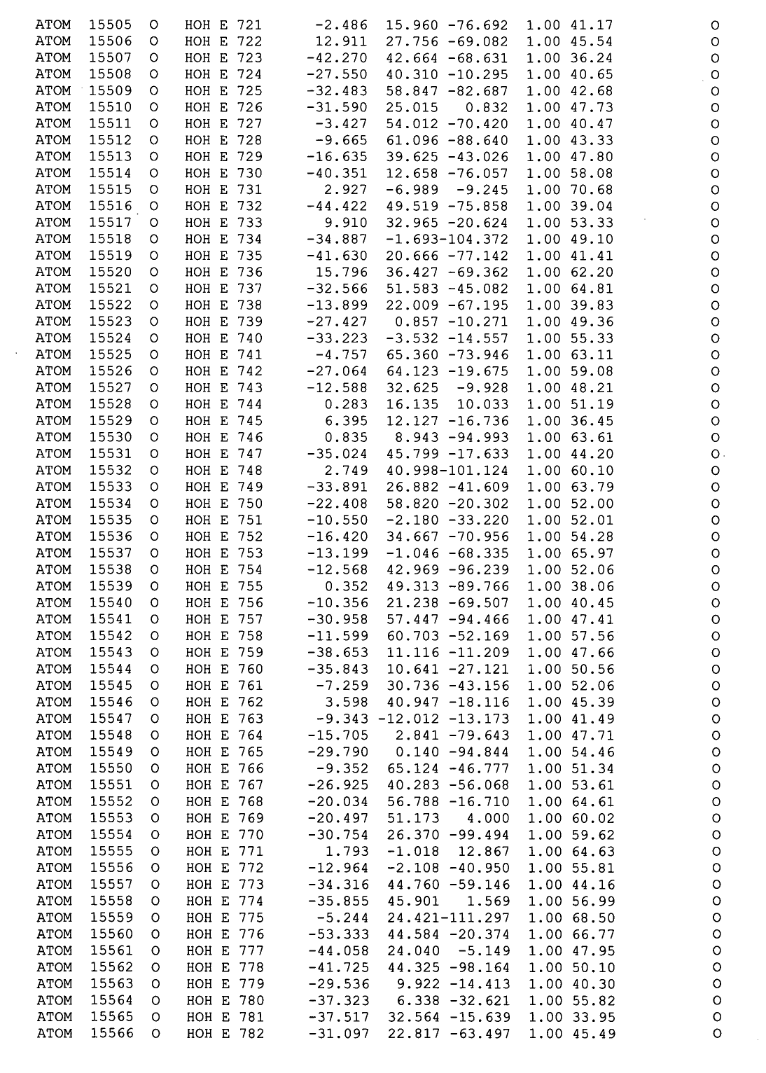

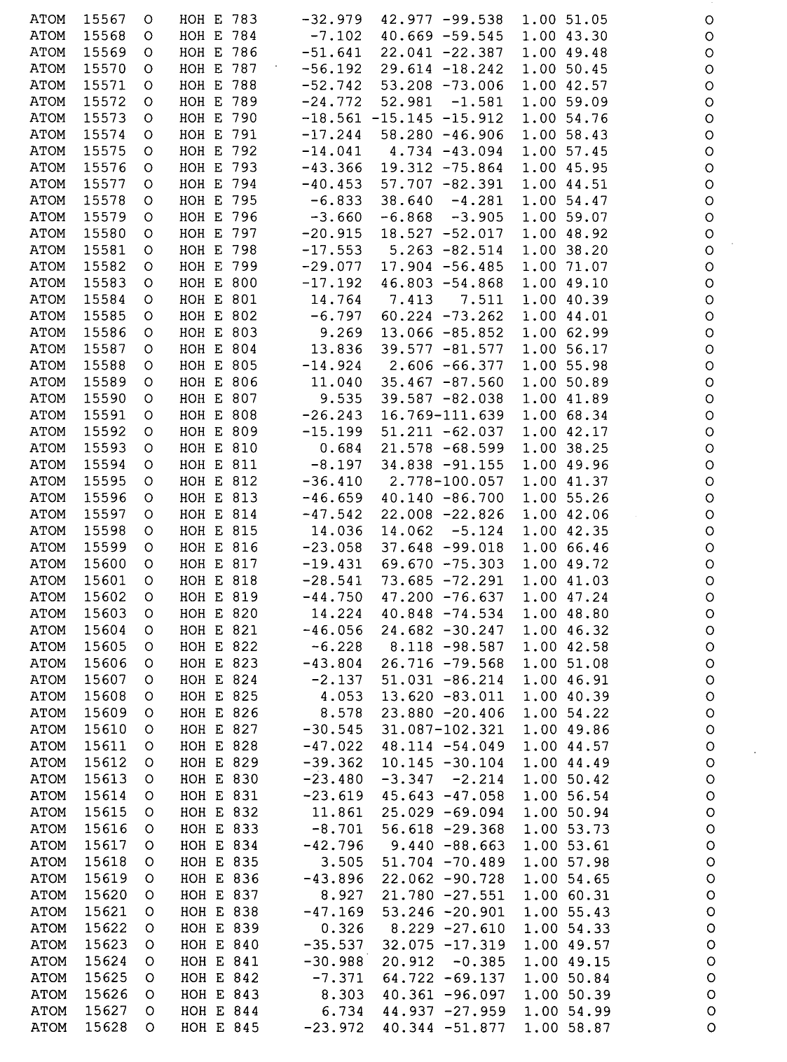

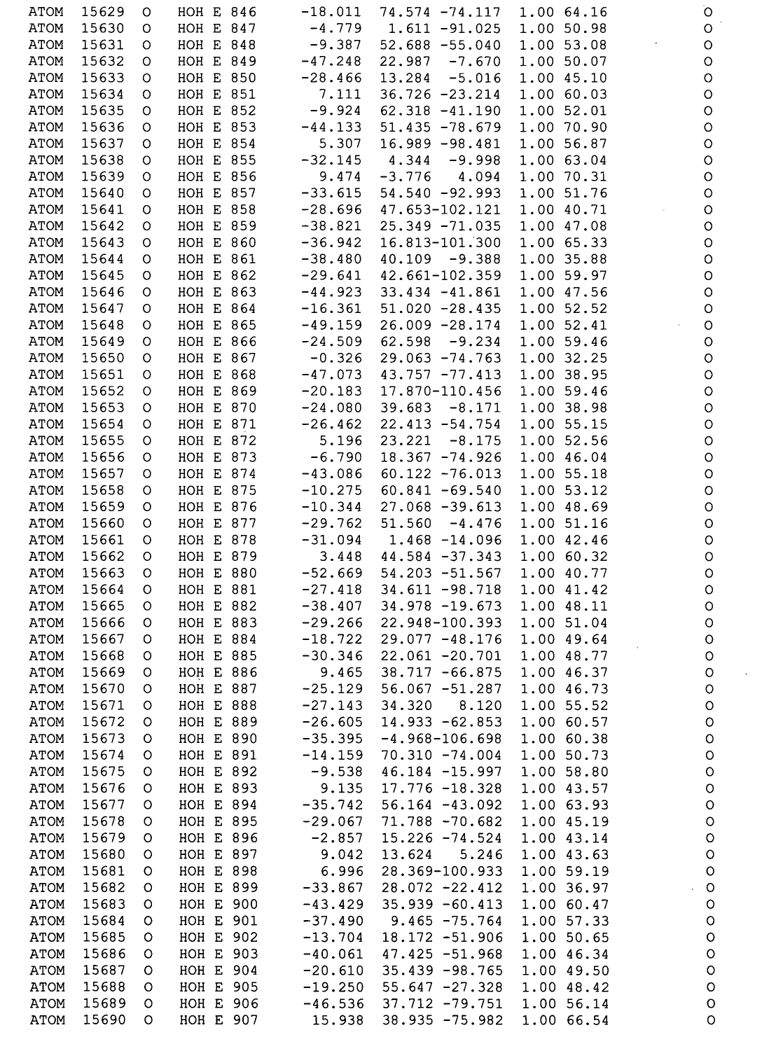

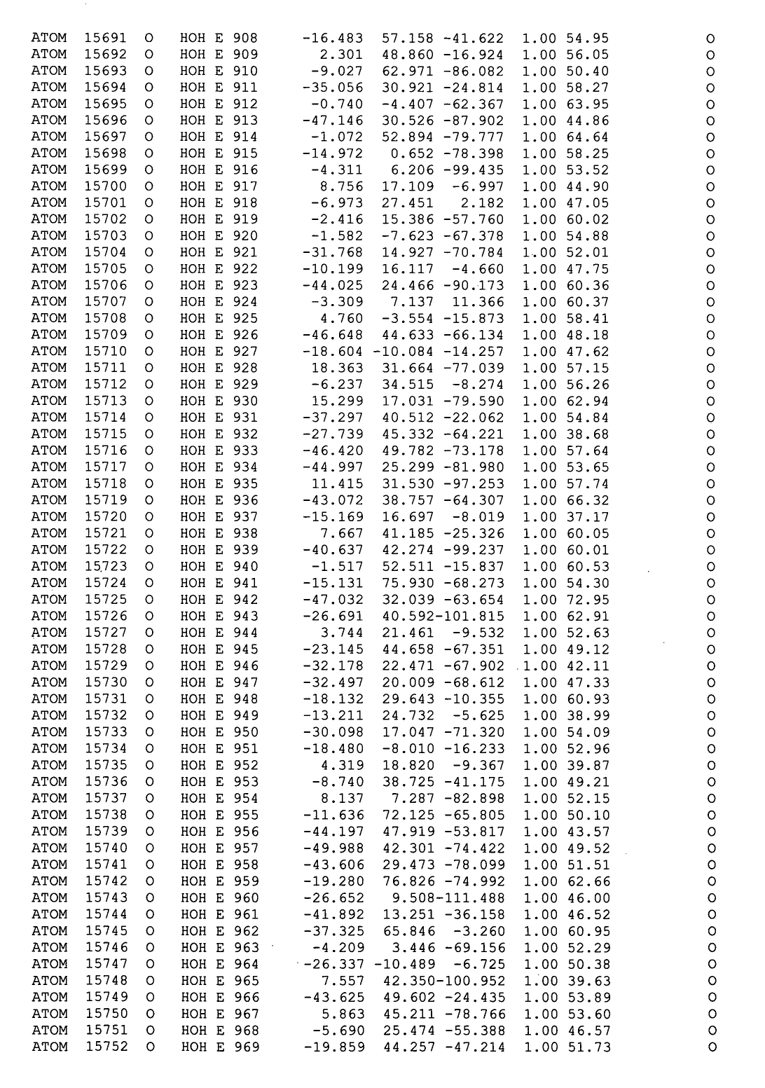

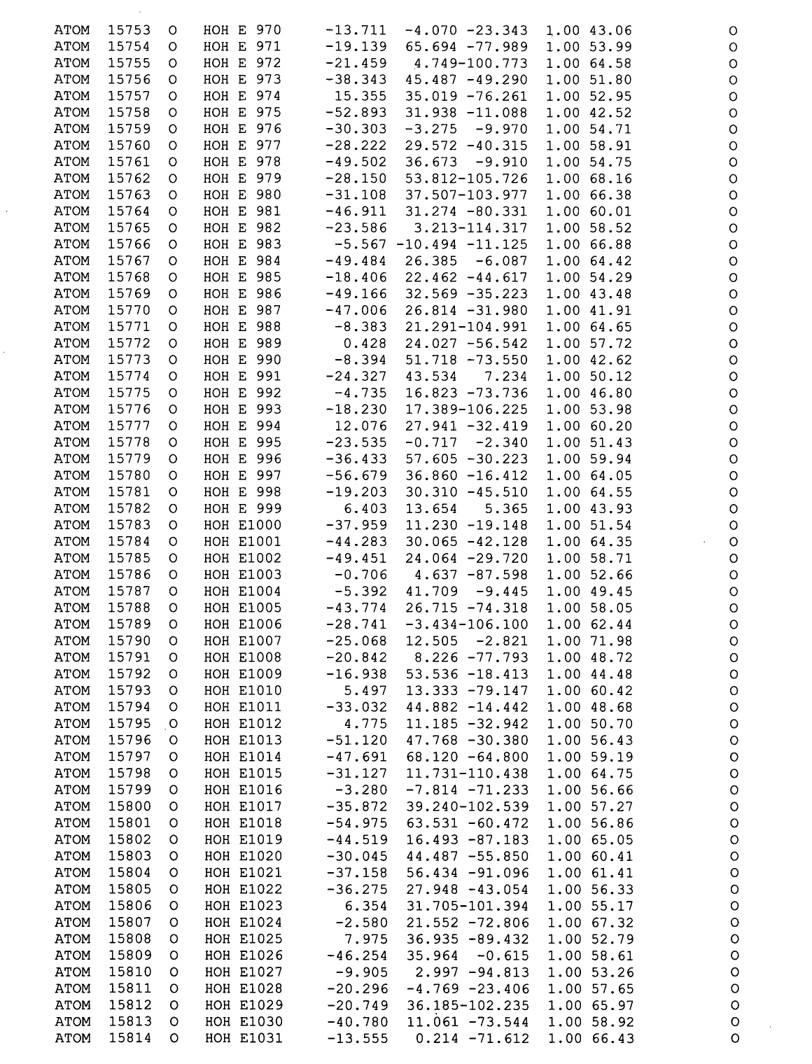

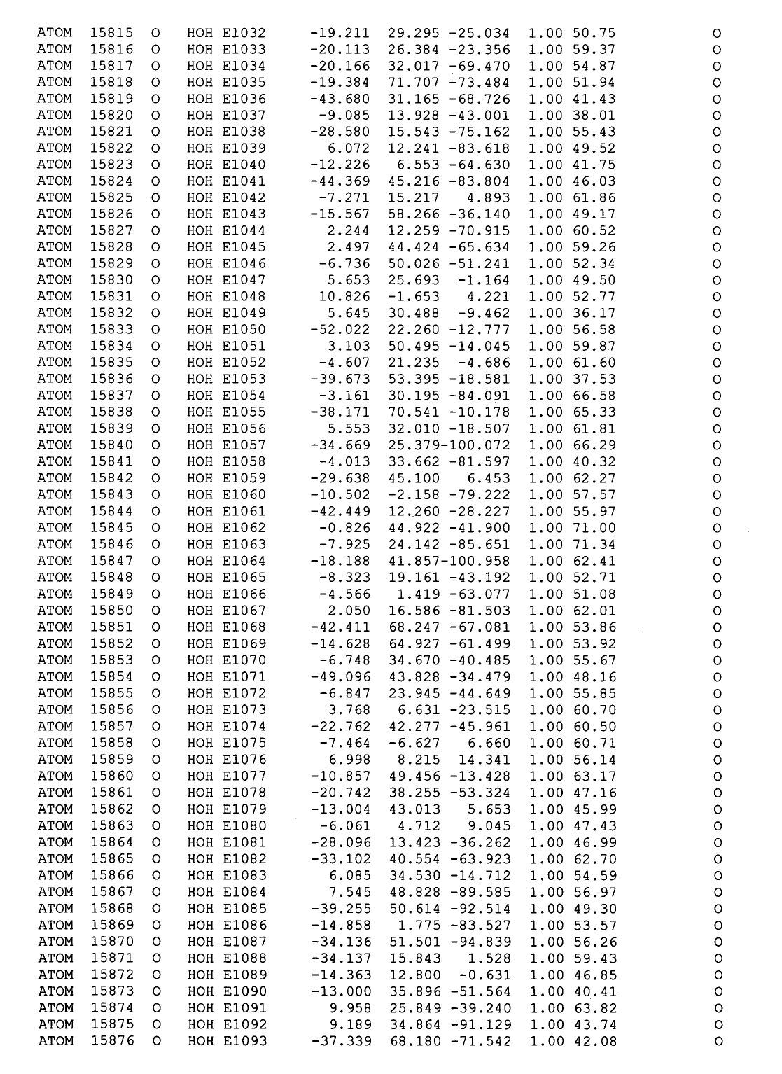

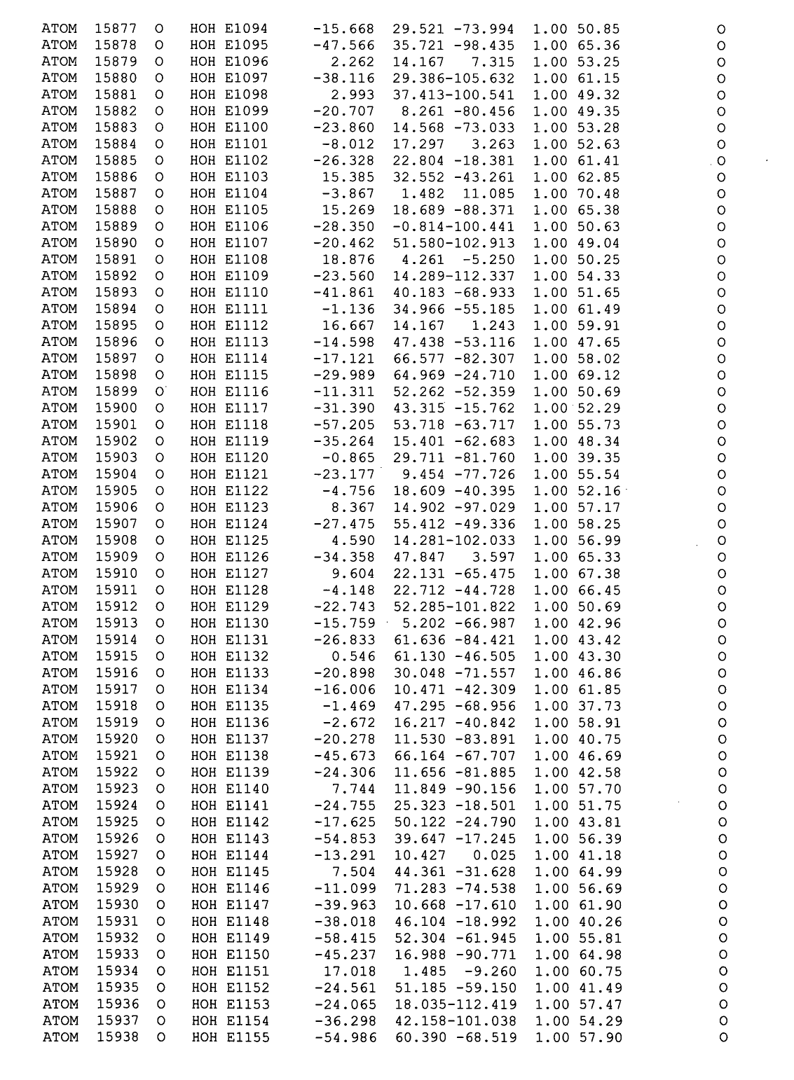

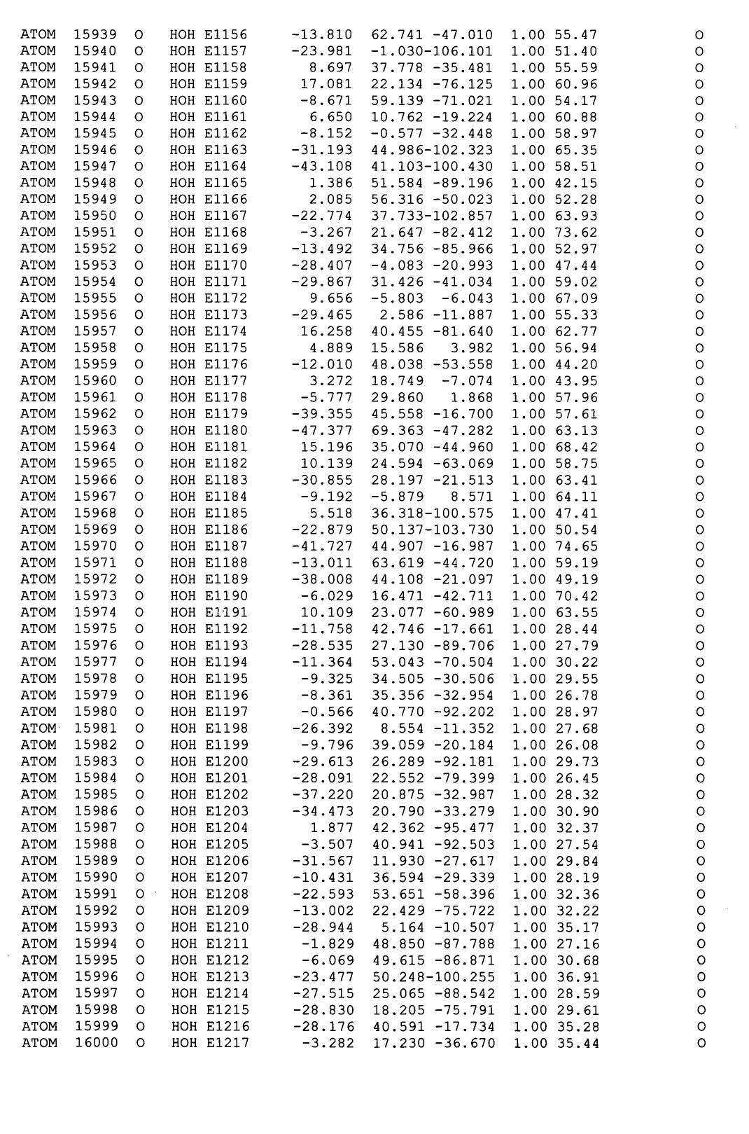

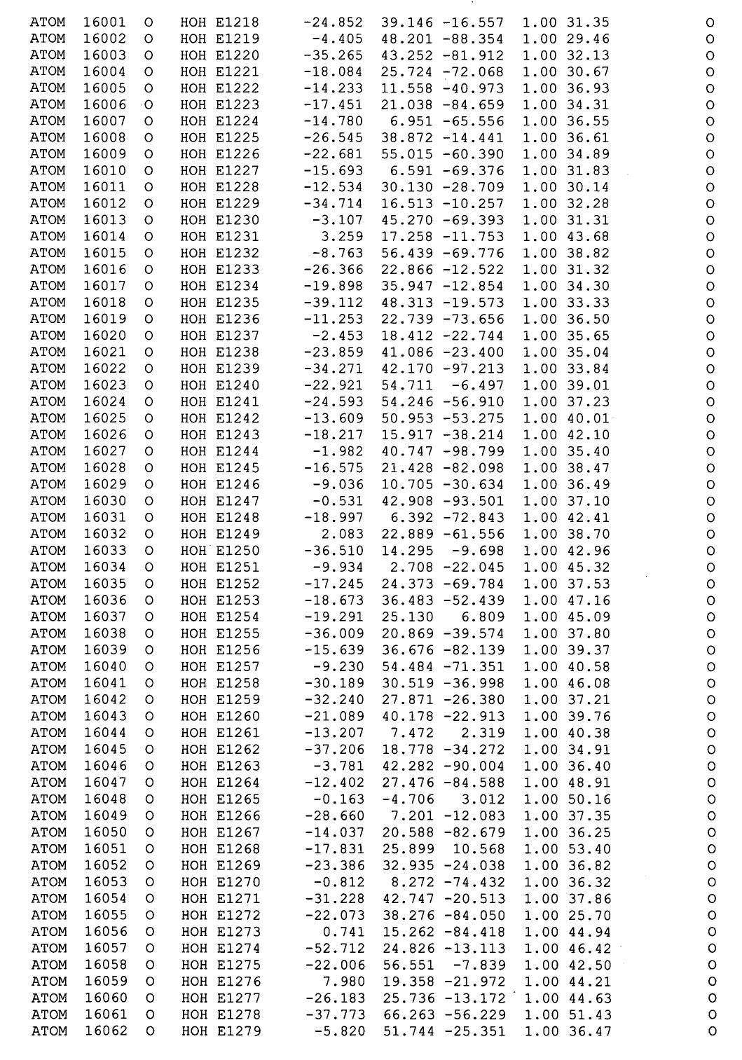

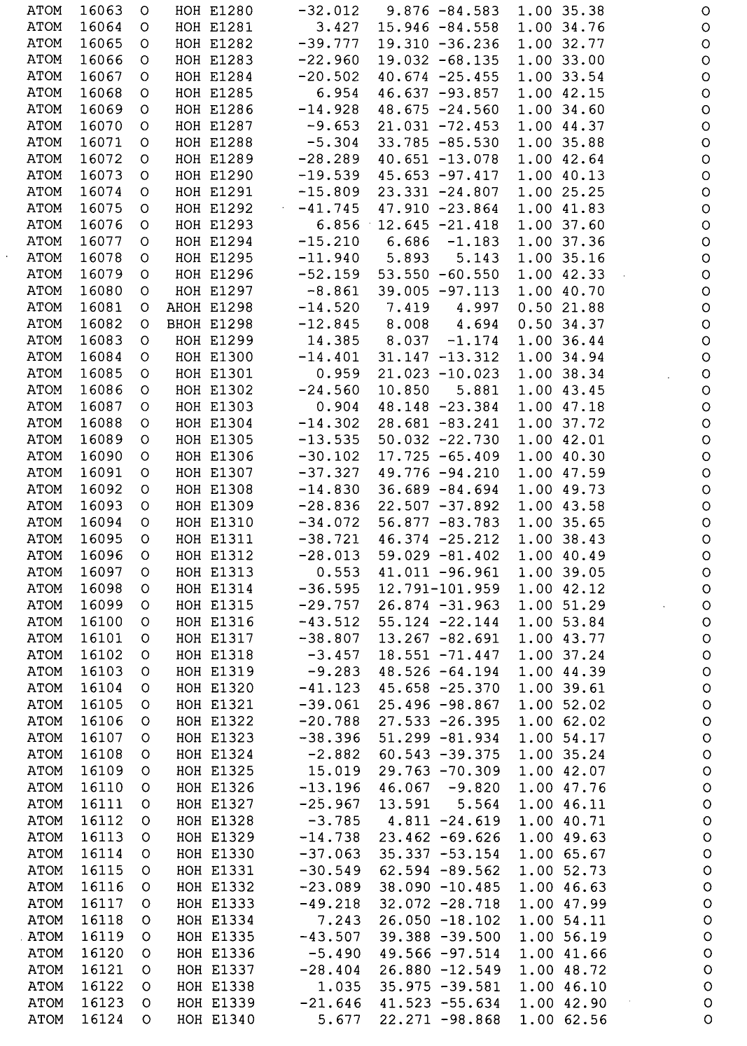

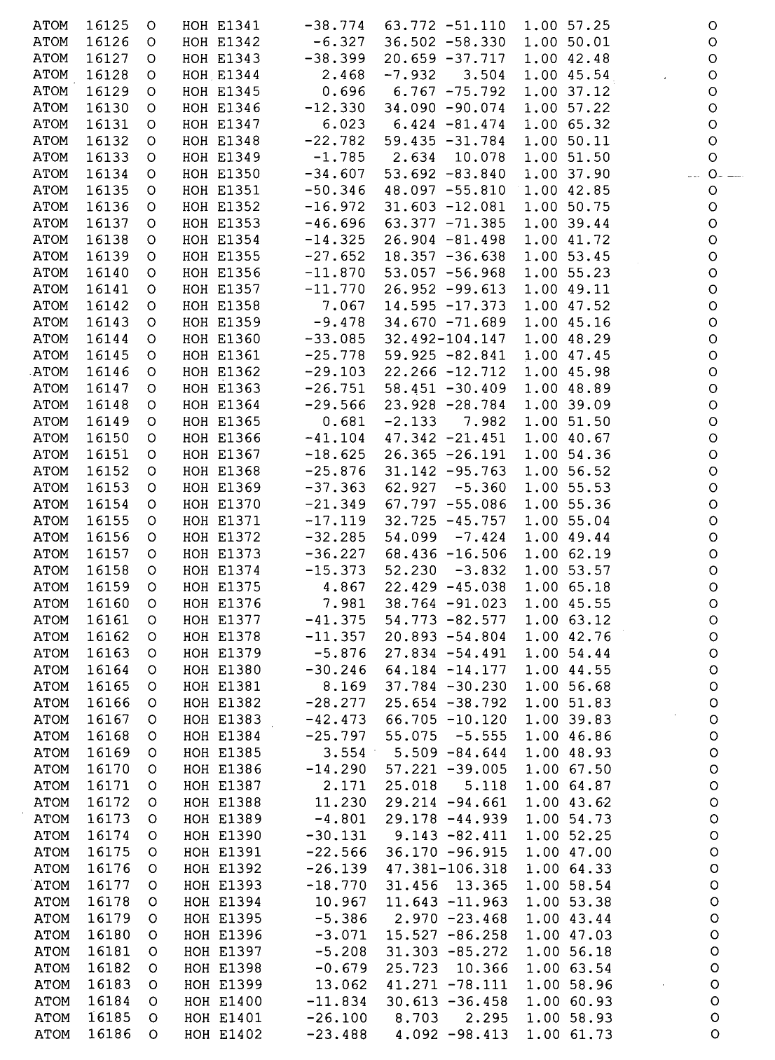

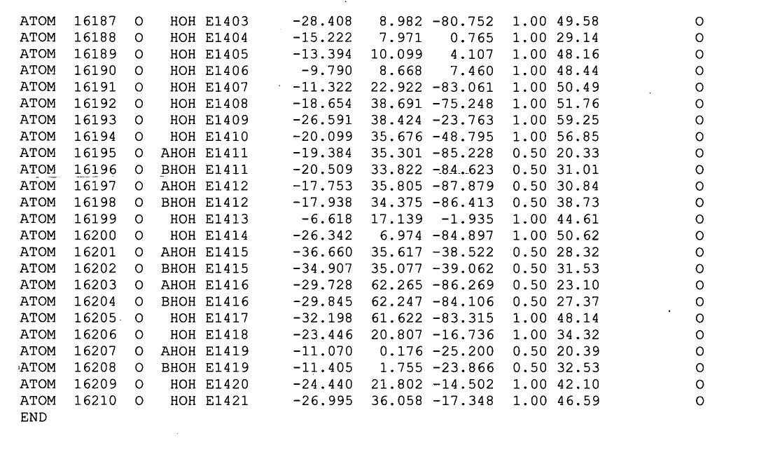

Analysis of the Co4-bound rPfA-M1 structure revealed that it is essentially identical to the inhibitor-free enzyme (r.m.s.d. of 0.13 Å over 890 Cy residues). The omit electron density of Co4 within the active site was well-defined (FIG. 5) and shows that the inhibitor slots neatly into the large catalytic cavity without causing any localised conformational shifts (FIG. 2D & FIG. 2E). Most notably, no movement of Val₄₅₉, which immediately precedes the GAMEN motif, was observed. In E. coli Aminopeptidase N protein a methionine is present at this position and functions as a cushion to accept substrates (Ito, K. et al. J Biol Chem 281, 33664-76 (2006)). Co4, however, makes several contacts within the PfA-M1 active site which accounts for its potent inhibitory property. The compound interacts with the catalytic zinc via the O-atoms of the central PO₂ group, and its phosphoryl O-atoms (O₃ and O₄) form hydrogen bonds with the side-chain of Tyr₅₈₀ (FIG. 2E). A cis-peptide (Glu₃₁₆-Ala₃₂₀) allows the side-chain of Glu₃₁₉ to extend into the active site, where it forms a hydrogen bond with the amino group (NH₂) of Co4 (FIG. 2E). The side-chain of Glu₄₆₃ and main-chain amide of Gly₄₆₀, both part of the GAMEN recognition motif, form H-bonds with the amino group (NH₂) of Co4 and the O₁ atom of Co4 respectively (FIG. 2E). The two Phe-rings of Co4 form relatively few interactions; however, the first Phe ring (C₁-C₅) packs against side-chains of residues Arg₄₈₉, Thr₄₉₂ and Val₄₉₃ while the second Phe ring (C₁₀-C₁₄) forms hydrophobic contacts with side-chains of Glu₃₁₇, Val₄₅₉, Met₄₆₂ (GAMEN motif), Tyr₅₇₅ and Met₁ₒ₃₄.

### EXAMPLES

Various aspects of the invention will now be described with reference to the following non-limiting examples and outline of the experimental procedures.

### Parasites and Preparation of Parasite Extract

P. falciparum clone D10 was cultured as described (Trager, W. & Jensen, J. B. Science 193, 673-5 (1976)). For experiments investigating the stage specific expression of PfA-M1, parasites were synchronized using two rounds of sorbitol treatment (Lambros, C. & Vanderburg, J. P. J. Parasitol 65, 418-420 (1979)), and stage specific parasites harvested at ring, trophozoite and schizont stage.

The P. falciparum M1 Alanyl Aminopeptidase Gene, Codon Optimization, and Gene Synthesis.

The M1 alanyl aminopeptidase gene sequence (MAL13P1.56) also known as PfA-M1 (Florent, I. et al. Mol Biochem Parasitol 97, 149-60 (1998)), as annotated by PlasmoDB, is located on chromosome 13 of P. falciparum and is a single copy gene. The gene is 3257 by in length and encodes a protein of 1085-amino acids with a predicted molecular mass of ˜126.064 kDa with an isoelectric point 7.64.

Expression and Purification of Recombinant Malarial M1 Alanyl Aminopeptidase (rPfA-M1) in E. coli.

A truncated form of the P. falciparum M1 aminopeptidase (residues 195-1085, rPfA-M1) was prepared by PCR amplification using the synthesized gene as a template followed by directional cloning into the bacterial expression vector pTrcHis2B (Invitrogen). The primers used were M1 forward 5′-CTGCAGAACCAAAGATCCAC-3′, and M1 reverse 5′-GGTACCTCAATGATGATGATGATGATGTGGGCCCAACTTGTTTGT-3′. Unique PstI and KpnI sites (underlined) were introduced at the 5′ and 3′ ends of the amplified product. A C-terminal His-tag was introduced into the M1 reverse primer (italics).

### Enzymatic Analysis

Aminopeptidase activity was determined by measuring the release of the fluorogenic leaving group, 7-amino-4-methyl-coumarin (NHMec) from the fluorogenic peptide substrates H-Leu-NHMec, H-Ala-NHMec, H-Arg-NHMec, H-Met-NHMec, H-Phe-NHMec, H-Gly-NHMec, H-Val-NHMec, H-Ile-NHMec and H-Pro-NHMec. Reactions were carried out in 96-well microtitre plates (200 μl total volume, 30 min, 37° C.) using a spectrofluorimeter (Bio-Tek KC4) with excitation at 370 nm and emission at 460 nm. Enzyme was first added to 50 mM Tris-HCl pH 8.0 before the addition of 10 μM H-Leu-NHMec. Initial rates were obtained at 37° C. over a range of substrate concentrations spanning K_{M} (0.2-500 μM) and at fixed enzyme concentrations in 50 mM Tris-HCl, pH 8.0. Inhibition experiments were carried out in the presence of substrate.

### Construction of PfA-M1 Transgenic Expression Plasmids and Transfection of Malaria Parasite

PCR forward primers for the truncated sequences (ggatccatgccaaaaatacattataggaaagattat) were designed against the PfA-M1 gene (MAL13P1.56) and contained a BamHI restriction site (highlighted in bold). A reverse primer (ctgcagtaat-ttatttgttaatc) contained a PstI site with the putative stop codon removed to facilitate the addition of a sequence encoding the cmyc reporter tag. PCR products were cloned into pGEM using a TA cloning system (Promega, USA) and sequenced to confirm that no Taq associated errors had occurred. Selected clones were digested out of the pGEM vector using BamHI and PstI and subcloned into the Gateway™ compatible entry vector pHcmycB (Gateway, InvitroGen) which had previously been digested using the same enzymes. A cmyc-tag was ligated in-frame at the 3′ end of the introduced gene sequence, respectively (Gardiner, D. L., Trenholme, K. R., Skinner-Adams, T. S., Stack, C. M. & Dalton, J. P. J Biol Chem 281, 1741-5 (2006)). These introduced genes were under the control of the HSP86 promoter. Using those entry vectors and Gateway™ compatible destination vectors with a destination cassette and a second cassette containing the human dihydrofolate reductase synthase gene under the control of the P. falciparum calmodulin promoter as a selectable marker, clonase reactions were then performed. The final plasmid, designated pHTB-PfA-M1-cmycB (cmyc-tag) was transfected into ring stage parasites by electroporation as described (Spielmann, T., Gardiner, D. L., Beck, H. P., Trenholme, K. R. & Kemp, D. J. Mol Microbiol 59, 779-94 (2006). Parasites resistant to WR99210 were obtained up to 25 days later.

### Northern Blotting

Total RNA was extracted and Northern blotting performed essentially as described by Kyes et al. (2000) with the following modifications: 100 μL pellet volumes of infected red blood cells were collected from cultures at approximately 5% parasitemia, lysed and stored in TRIzol (Invitrogen, U.S.A). Samples were separated on a 1% TBE agarose gel containing 10 mM guanidine thiocynate (Sigma-Aldrich, Australia), soaked in 50 mM NaOH for 30 minutes and transferred onto a Hybond N+ membrane (Amersham Biosciences, U.K.).

Blots were probed with a 1500 by PCR product amplified from a full length PfA-M1 pGem clone using primers PfA-M1IntF (tacaatgggctttagaatgtc), and PfA-M1 IntR (aattcatcatcttttga). This product was labelled with α-³²P-dCTP by random priming using a Decaprime II kit (Ambion, U.S.A. The probe was hybridized overnight at 40° C. in a hybridization buffer containing formamide (Northern Max; Ambion). The filter was washed once at low stringency and twice at high stringency (Northern Max; Ambion), then exposed overnight to Super Rx Medical X-Ray film (Fuji, Japan), and developed using a Kodak X-OMAT 3000RA processor (Kodak, Australia).

### Immunoblotting

Parasite protein fractions were extracted using 0.03% saponin (Sigma-Aldrich Australia) and prepared as described previously (Spielmann, T., Gardiner, D. L., Beck, H. P., Trenholme, K. R. & Kemp, D. J. Mol Microbiol 59, 779-94 (2006)). SDS-PAGE was performed using 10% acrylamide gels and run on Miniprotein II rigs (BioRad, U.S.A). Equal loading was estimated using the Bradford method (Bradford, M. M. Anal. Biochem 72, 248-254 (1976)), and by staining gels with Coomassie Brilliant Blue (Bio-rad, U.S.A) with protein proportions visually estimated.

Protein was transferred onto Hybond C+ membranes (Amersham Biosciences, U.K.), which were blocked in 5% skim milk powder for 1 hour at 37° C. or overnight at 4° C. Anti-cmyc (Sigma-Aldrich, Australia) were used as primary antibodies to label transgenic PfA-M1 protein at a 1/3000 dilution. The secondary antibody was an anti-mouse IgG (Chemicon, Australia) used at a dilution of 1/5000. Blots were incubated with ECL Detection Reagents (Amersham Biosciences, U.K.), with exposure times ranging from 5-10 minutes.

### In Vitro Sensitivity to Aminopeptidase Inhibitors

The in vitro sensitivity of each parasite population to Co4 was determined using [³H]-hypoxanthine incorporation (Geary, T. G., Delaney, E. J., Klotz, I. M. & Jensen, J. B. Mol Biochem Parasitol 9, 59-72 (1983)). Briefly, serial dilutions of each inhibitor were prepared in culture media (0.2-200 μM) and added with [³H]-hypoxanthine (0.5 μCi/well) to asynchronous cultures. After a 48 hr incubation the amount of [³H]hypoxanthine incorporation was measured IC₅₀ values were determined by linear interpolation of inhibition curves (Huber, W. & Koella, J. C. Acta Trop 55, 257-61 (1993)). Each assay was performed in triplicate on at least two separate occasions.

### Crystallization, X-Ray Data Collection, Structure Determination and Refinement

rPfA-M1 was extracted and purified from BL21 cells by Ni NTA-agarose chromatography (Stack, C. M. et al. J Biol Chem 282, 2069-80 (2007)). The eluted enzyme was dialyzed against gel filtration buffer (50 mM Hepes pH 8.5; 300 mM NaCl 5% (v/v) glycerol) before size-exclusion chromatography using a Superdex S200 10/30 column. Before crystallization, purified enzyme were concentrated to 5 mg/mL. The crystals were grown using the hanging drop vapour diffusion method, with 1:1 (v/v) ratio of protein to mother liquor (0.5 ml well volume). The crystals appeared overnight in 22% (v/v) polyethylene glycol 8000, 10% (v/v) glycerol, 0.1 M Tris (pH 8.5) and 0.2 M magnesium chloride and reached full size in 3 days. Crystals of the rPfA-M1-Co4 complex were obtained by cocrystallisation under similar conditions in the presence of the ligand at 1 mM. Crystals were dehydrated against reservoir buffer with 15% (v/v) glycerol for 16 hours. Crystals were equilibrated for 5 min in reservoir buffer in the presence of 20% (v/v) glycerol. Cryoannealing was performed three times by blocking the cryostream (100 K) for 5 seconds. Cryoannealing substantially improved the diffraction quality observed. Crystal quality was variable and a large number had to be screened.

Data were collected in-house on a Rikagu RU-3HBR rotating anode generator with helium purged OSMIC focussing mirrors as an X-ray source. Data are collected using an R-AXIS IV++ detector. The diffraction data for the ligand-free and Co4-bound protease were collected to 2.1 and 2.0 Å resolution, respectively. Diffraction images were processed using MOSFLM (Leslie, A. G. W. in Joint CCP4+ESF-EAMCB Newsletter on Protein Crystallography, No. 26. (1992)) and SCALA (Evans, P. Acta Crystallogr D Biol Crystallogr 62, 72-82 (2006)) from the CCP4 suite (CCP4. Acta Crystallogr D50, 760-763 (1994)). 5% of each dataset was flagged for calculation of R_{Free} (Brunger, A. T. Acta Crystallogr D Biol Crystallogr 49, 24-36 (1993)) with neither a sigma nor a low-resolution cut-off applied to the data. A summary of statistics is provided in Table 3. Subsequent crystallographic and structural analysis was performed using the CCP41 interface (Potterton, E., Briggs, P., Turkenburg, M. & Dodson, E. Acta Crystallogr D Biol Crystallogr 59, 1131-7 (2003)) to the CCP4 suite (Evans, P. Acta Crystallogr D Biol Crystallogr 62, 72-82 (2006)), unless stated otherwise. Structure solution preceded using the Molecular Replacement method and the program PHASER (McCoy, A. J., Grosse-Kunstleve, R. W., Storoni, L. C. & Read, R. J. Acta Crystallogr D Biol Crystallogr 61, 458-64 (2005)). A search model was constructed from the crystal structure of aminopeptidase N from Neisseria meningitides (PDB 2GTQ), the closest structural homolog identified using the FFAS server (Jaroszewski, L., Rychlewski, L., Li, Z., Li, W. & Godzik, A. Nucleic Acids Res 33, W284-8 (2005)). A “mixed” model consisting of conserved sidechains (all other non alanine/glycine residues truncated at Cγ atom) was then created using the SCRWL server (Jaroszewski, L., Rychlewski, L., Li, Z., Li, W. & Godzik, A. Nucleic Acids Res 33, W284-8 (2005)).

Maximum likelihood refinement using REFMAC (Murshudov, G. N., Vagin, A. A. & Dodson, E. J. Acta Crystallographica D53, 240-255 (1997)), incorporating translation, liberation and screw-rotation displacement (TLS) refinement was carried out, using a bulk solvent correction (Babinet model with mask). Imposed restraints were guided by manual inspection of the model and R_{Free}. Simulated annealing composite omit maps were generated using CNS (Brunger, A. T. et al. Acta Crystallogr D Biol Crystallogr 54 (Pt 5), 905-21 (1998)) omitting 5% of the model. All model building and structural validation was done using COOT (Emsley, P. & Cowtan, K. Acta Crystallogr D Biol Crystallogr 60, 2126-32 (2004)). Water molecules were added to the model using ARP/wARP (Cohen, S. X. et al. Acta Crystallogr D Biol Crystallogr 64, 49-60 (2008)) when the R_{free} reached 25%. Solvent molecules were retained only if they had acceptable hydrogen-bonding geometry contacts of 2.5 to 3.5 Å with protein atoms or with existing solvent and were in good 2Fₒ-Fₒ and Fₒ-F_{c} electron density.

The coordinates and structure factors are being deposited in the Protein Data Bank.

### Structural Analysis and Figures

Pymol were used to produce all structural representations (http://www.pymol.org). CCP4MG (CCP4, 1994) was used to produce FIG. 2D. Surfaces in FIG. 2C were color coded according to electrostatic potential (calculated by the Poisson-Boltzmann solver within CCP4MG). Lys and Arg residues were assigned a single positive charge, and Asp and Glu residues were assigned a single negative charge; all other residues were considered neutral. The calculation was done assuming a uniform dielectric constant of 80 for the solvent and 2 for the protein interior. The ionic strength was set to zero. The shading of the surface represents the electrostatic potential at the protein surface, going from black (potential of +10 kT/e) to grey (potential of −10 kT/e), where T is temperature, e is the charge of an electron, and k is the Boltzmann constant. The probe radius used was 1.4 Å. Hydrogen bonds (excluding water-mediated bonds), were calculated using the CONTACT (CCP4. Acta Crystallogr D50, 760-763 (1994))

The word ‘comprising’ and forms of the word ‘comprising’ as used in this description does not limit the invention claimed to exclude any variants or additions.

Modifications and improvements to the invention will be readily apparent to those skilled in the art. Such modifications and improvements are intended to be within the scope of this invention.

What is claimed is:

1. A structure of PfA-M1 as defined by coordinates chosen from the group comprising Table A or Table B.

2. A structure of PfA-M1 as defined by the coordinates listed in Table A.

3. A structure of PfA-M1 in complex with Co4 as defined by the coordinates listed in Table B.

4. A crystal of PfA-M1 consisting of a primitive orthorhombic P2₁2₁2₁ space group with unit cell dimensions of a=75.7±2.1 Å, b=108.7±2.1 Å and c=118.0±2.1 Å.

5. A crystal of PfA-M1 in complex with Co4 consisting of a primitive orthorhombic P2₁2₁2₁ space group with unit cell dimensions of a=75.9±2.0 Å, b=108.6±2.0 Å and c=118.3±2.0 Å.

6. A machine-readable data storage medium which comprises a data storage material encoded with machine readable data defined by the structure coordinates of PfA-M1 chosen from the group comprising Table A or Table B or coordinates defining homologues of the structure.

7. A machine-readable data storage medium which comprises a data storage material encoded with machine readable data defined by the structure coordinates of PfA-M1 according to Table A or a homologue of this structure.

8. A machine-readable data storage medium which comprises a data storage material encoded with machine readable data defined by the structure coordinates of PfA-M1 in complex with Co4 according to Table B or a homologue of this structure

9. A method of using the structure of claim 1 as a structural model.

10. A method of using the structural model according to claim 9 for high-throughput chemical screening.

11. The method of using the structural model according to claim 9 for the identification of one or more anti-malarials or their homologues.

12. An antimalarial drug identified by the use according to claim 9.

13. The use according to claim 9 for determining at least a portion of the three-dimensional structure of a molecular species.

14. The use according to claim 9 for the identification of one or more molecular species that modulate PfA-M1 to inhibit at least part of its activity.

15. The use according to claim 7 for the identification of one or more molecular species that modulate the Co4-bound PfA-M1 complex to inhibit at least part of its activity.

16. A method for screening a molecular species for anti-malarial activity comprising the steps of:
(i) characterising an active site from the structure coordinates chosen from the group comprising Table A and Table B;
(ii) identifying candidate molecular species that interact with at least part of the active site cavity; and
(iii) obtaining or synthesizing said molecular species.

17. The method according to claim 16 wherein the molecular species interacts with a C-terminal domain IV opening of the active site cavity.

18. The method according to claim 16 wherein the molecular species interacts with a groove at the junction of domains I and IV of the active site cavity.

19. A method for screening molecular species for anti-malarial activity comprising the steps of:
characterising an active site from structure coordinates chosen from the group comprising Table A or Table B;
(ii) identifying molecular species that interact with one or more amino acids chosen from the group comprising Ala₃₂₀, Ala₄₆₁, Arg₄₈₉, GIn₃₁₇, Glu₃₁₉, Glu₄₆₃, Glu₅₁₉, Glu₄₉₇, Gly₄₆₀, His₄₉₆, His₅₀₀, Lys₅₁₈, Met₄₆₂, Met₁₀₃₄, Thr₄₉₂, Tyr₅₇₅, Tyr₅₈₀, Val₄₅₉ and Val₄₉₃,
(iii) obtaining or synthesizing said molecular species.

20. A method according to claim 19, wherein step (ii) includes interaction of the molecular species with one or more amino acid residues lining the active site of a malaria protease that are chosen from the group comprising 303-305; 314-325; 458-463; 489-526 (incorporating ‘catalytic residues’ His-496; His-500 and Glu-519); 570-582; and 1022-1038.

21. The method according to claim 19, wherein the molecular species is a molecule or molecular complex.

22. An anti-malarial drug identified using the method of claim 19.

23. An anti-malarial drug candidate identified or designed using the method of claim 19.

24. The anti-malarial drug according to claim 22, wherein said drug is used to block the entrance of Hb-derived peptides and/or block the exit of released amino acids at the active site of PfA-M1 protease.

25. The anti-malarial drug according to claim 22, wherein said drug is used to obstruct protein metabolism and synthesis in a parasite.

26. A method of killing a parasite using an antimalarial drug according to claim 22.
